(19) United States
(12) Patent Application Publication
(10) Pub. No.: US 2025/0154114 A1
(43) Pub. Date: May 15, 2025

(54) MATERIAL FOR METAL PATTERNING, HETEROCYCLIC COMPOUND, THIN FILM FOR METAL PATTERNING, ORGANIC ELECTROLUMINESCENT DEVICE, ELECTRONIC DEVICE, AND METHOD FOR FORMING METAL PATTERN

(71) Applicant: TOSOH CORPORATION, Yamaguchi (JP)

(72) Inventors: Hiroyuki KAWASHIMA, Kanagawa (JP); Kenji KOIKE, Kanagawa (JP); Naoki MATSUMOTO, Kanagawa (JP); Shintaro NOMURA, Kanagawa (JP); Eriko OTA, Kanagawa (JP); Kazuki HATTORI, Kanagawa (JP)

(73) Assignee: TOSOH CORPORATION, Yamaguchi (JP)

(21) Appl. No.: 18/835,842

(22) PCT Filed: Feb. 9, 2023
(86) PCT No.: PCT/JP2023/004430

§ 371 Date: Aug. 5, 2024

(30) Foreign Application Priority Data
Feb. 9, 2022 (JP) 2022-018503
Nov. 30, 2022 (JP) 2022-192376
Feb. 9, 2023 (JP) 2023-018054

(51) Int. Cl. C07D 251/30; B05D 1/32; C07D 213/26; C07D 239/26; C07D 241/12; C07D 251/20; C07D 401/04; C07D 401/10; C07D 401/12; C07D 401/14; C07D 403/04; C07D 403/10; C07D 403/12; C07D 403/14; C07D 405/04; C07D 405/14; C07D 409/10; C07D 409/14; C07D 471/04; C07D 471/08; C07D 487/04; H10K 50/813; H10K 71/60

(52) U.S. Cl. CPC C07D 251/30; B05D 1/32; C07D 213/26; C07D 239/26; C07D 241/12; C07D 251/20; C07D 401/04; C07D 401/10; C07D 401/12; C07D 401/14; C07D 403/04; C07D 403/10; C07D 403/12; C07D 403/14; C07D 405/04; C07D 405/14; C07D 409/10; C07D 409/14; C07D 471/04; C07D 471/08; C07D 487/04; H10K 50/813; H10K 71/60

(57) ABSTRACT

To provide a material for metal patterning capable of significantly suppressing the formation of a thin metal film on a film surface, a heterocyclic compound, a thin film for metal patterning using them, an organic electroluminescent element, a method for forming a metal pattern, and an electronic device. A material for metal patterning represented by the formula (A1) or (B1):

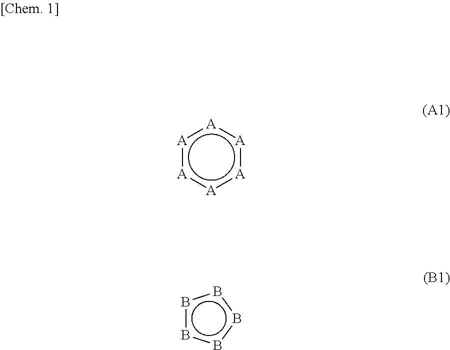

wherein A each independently denotes N or CR, at least one thereof being N and at least one thereof being CR; and B denotes N, NR, S, O or CR, at least one thereof being N, S or O and at least one thereof being CR.

### TECHNICAL FIELD

The present disclosure relates to a material for metal patterning, a heterocyclic compound, a thin film for metal patterning, an organic electroluminescent element, an electronic device, a method for forming a metal pattern and a heterocyclic compound.

### BACKGROUND ART

In recent years, organic electronic devices, such as organic electroluminescent (EL) elements, organic thin-film solar cells, organic transistors and organic sensors, have been widely developed. In an organic electronic device, a thin metal film is used as an electrode, and the thin metal film needs to be patterned into a desired shape.

A method known as a method for patterning a metal electrode includes patterning, as a base layer, a material for metal patterning in which adhesion of a metal is suppressed and vapor-depositing a metal on the base layer. In this method, a metal film is selectively formed on a portion where the film of the material for metal patterning is not formed, and a metal electrode patterned into a desired shape can therefore be formed.

Patent Literature 1 discloses a technique of patterning a magnesium metal using an anthracene derivative as a material for metal patterning.

### CITATION LIST

### Patent Literature

PTL 1: International Publication No. WO 2020/225778

### SUMMARY OF INVENTION

### Technical Problem

In the method according to Patent Literature 1, however, it is difficult to form a pattern while significantly suppressing adhesion of a metal to a portion other than a desired portion. Furthermore, patterning of a metal other than magnesium is difficult with the compound described in Patent Literature 1.

Accordingly, one aspect of the present disclosure is directed to providing a material for metal patterning capable of significantly suppressing the formation of various thin metal films on a film surface, a thin film for metal patterning using the material for metal patterning, an organic electroluminescent element, a method for forming a metal pattern an electronic device and a heterocyclic compound.

### Solution to Problem

According to one aspect of the present disclosure, the present invention has been completed by finding a material for metal patterning represented by the formula (A1) or (B1).

Thus, the present invention resides in the following [1] to [27].

[1]

A material for metal patterning represented by the formula (A1) or (B1):

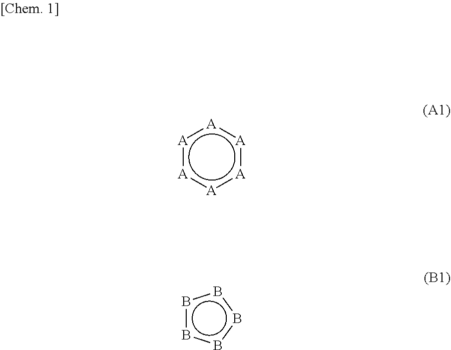

wherein
A each independently denotes N or CR, at least one thereof being N and at least one thereof being CR;
B each independently denotes N, S, O or CR, at least one thereof being N, S or O and at least one thereof being CR;
R each independently denotes the formula (A01) or the formula (A02), at least one thereof being the formula (A01):

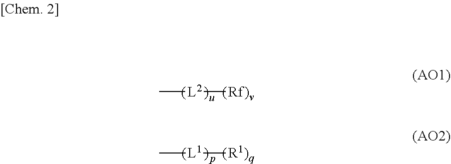

L¹ and L² each independently denote
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR²;
Rf each independently denotes
a monovalent or divalent substituent with 1 or more carbon atoms containing 3 or more fluorine atoms;
R¹ and R² each independently denote an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent or divalent heteroaliphatic hydrocarbon group with 3 to 18 carbon atoms, or
H, D (deuterium atom) or F (fluorine atom); and
p each independently denotes an integer in the range of 0 to 12.
q each independently denotes an integer in the range of 1 to 6.
u each independently denotes an integer in the range of 0 to 12.
V each independently denotes an integer in the range of 1 to 6.
   [2]

The material for metal patterning according to [1], wherein Rf is represented by the following formula (001):

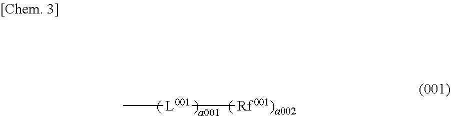

wherein
Rf⁰⁰¹ each independently denotes
a linear, branched or cyclic aliphatic hydrocarbon group with 1 to 18 carbon atoms containing 3 or more fluorine atoms;
L⁰⁰¹ each independently denotes
a linear, branched or cyclic divalent aliphatic hydrocarbon group with 1 to 18 carbon atoms optionally substituted with O, S or a fluorine atom, or
a vinylene group that may be substituted with fluorine and may form a ring;
a001 each independently denotes an integer in the range of 0 to 4; and
a002 each independently denotes an integer in the range of 1 to 4.
   [3]

The material for metal patterning according to [1], wherein u in at least one formula (A01) in the formula (A1) is 0, and u in at least one formula (A01) in the formula (B1) is 0.

[4]

The material for metal patterning according to [1], represented by any one of the formulae (101) to (105):

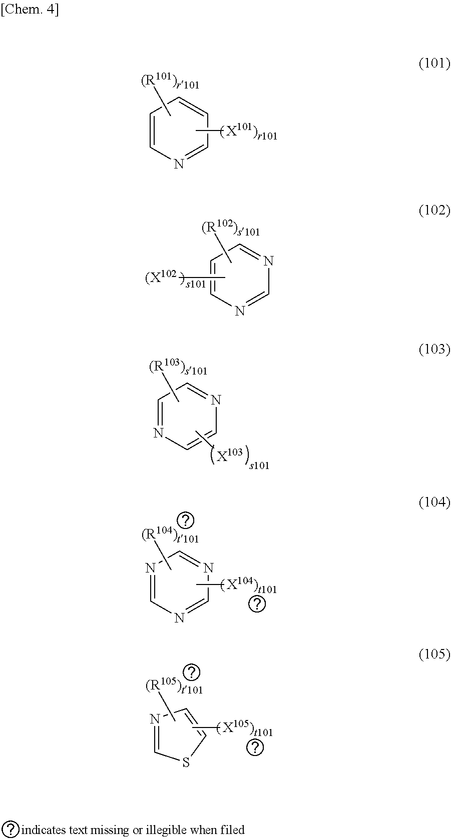

wherein
X¹⁰¹, X¹⁰², X¹⁰³, X¹⁰⁴ and X¹⁰⁵ are each independently represented by the formula (111):

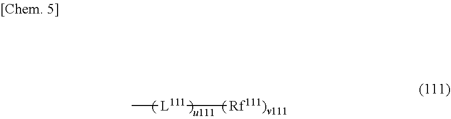

wherein
R¹⁰¹, R¹⁰², R¹⁰³, R¹⁰⁴ and R¹⁰⁵ are each independently represented by the formula (121):

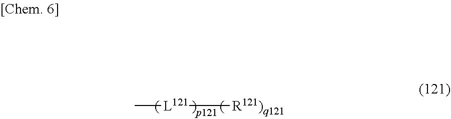

L¹¹¹ and L¹²¹ each independently denote
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR¹¹¹;
Rf¹¹¹ each independently denotes a structure represented by the formula (001);
R¹¹¹ and R¹²¹ each independently denote
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 3 to 18 carbon atoms, or
H, D (deuterium atom) or F (fluorine atom); and
r101 denotes an integer in the range of 1 to 5, and r′101 denotes an integer in the range of 0 to 4, provided that (r101+r′101) is an integer of 5 or less.
s101 each independently denotes an integer in the range of 1 to 4, and s′101 each independently denotes an integer in the range of 0 to 3, provided that (s101+s′101) is an integer of 4 or less.
t101 each independently denotes an integer in the range of 1 to 3, and t′101 denotes an integer in the range of 0 to 2, provided that (t101+t′101) is an integer of 3 or less.
u111 each independently denotes an integer in the range of 0 to 12.
v111 each independently denotes an integer in the range of 1 to 6.
p121 each independently denotes an integer in the range of 0 to 12.
q121 each independently denotes an integer in the range of 1 to 6.
u111 in at least one X¹⁰¹, at least one X¹⁰², at least one X¹⁰³, at least one X¹⁰⁴ and at least one X¹⁰⁵ each independently denotes 0.
   [5]

The material for metal patterning according to [4], wherein
when L¹¹¹ and L¹²¹ denote a substituted aromatic hydrocarbon group, a substituted heteroaromatic group, a substituted aliphatic hydrocarbon group or a substituted heteroaliphatic hydrocarbon group, these groups are each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a cyano group, a fluorine atom, a deuterium atom and a structure represented by the formula (001), and
a combination of these groups.
   [6]

The material for metal patterning according to [1], wherein
when R¹ and R² denote a substituted aromatic hydrocarbon group, a substituted heteroaromatic group, a substituted aliphatic hydrocarbon group or a substituted heteroaliphatic hydrocarbon group, these groups are each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a hydrogen atom, a fluorine atom, a deuterium atom and a structure represented by the formula (001), and
a combination of these groups.
   [7]

The material for metal patterning according to [4], wherein a ratio of the number of fluorine atoms to the number of carbon atoms in at least one Rf¹¹¹ is 50% or more.

[8]

The material for metal patterning according to [4], wherein R¹⁰¹, R¹⁰², R¹⁰³, R¹⁰⁴ and R¹⁰⁵ are each independently represented by the formula (131):

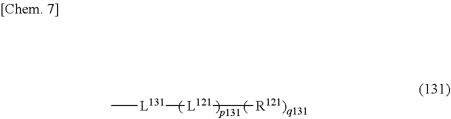

L¹²¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O, S or NR¹¹¹;
L¹³¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O or S;
R¹¹¹ and R¹²¹ each independently denote
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 3 to 18 carbon atoms, or
H, D (deuterium atom) or F (fluorine atom); and
p131 each independently denotes an integer in the range of 0 to 11.
q131 each independently denotes an integer in the range of 1 to 6.
   [9]

The material for metal patterning according to [8], wherein the monocyclic, linked or condensed divalent to tetravalent aromatic hydrocarbon group represented by L¹²¹ or L¹³¹ is phenyl or has a structure in which a plurality of benzene rings are linked or condensed.

[10]

The material for metal patterning according to [8], wherein the monocyclic, linked or condensed divalent to tetravalent heteroaromatic group represented by L¹²¹ or L¹³¹ has N, O or S as a heteroatom and is a 5-membered ring, a 6-membered ring or a condensed structure thereof.

[11]

The material for metal patterning according to [8], wherein
the monocyclic, linked or condensed divalent to tetravalent aromatic hydrocarbon group represented by L¹²¹ or L¹³¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene or dibenzochrysene,
the monocyclic, linked or condensed divalent to tetravalent heteroaromatic group is pyridine, pyrimidine, pyrazine, triazine, carbazole, furan, thiophene, benzofuran, benzothiophene, benzodioxin, dibenzofuran, dibenzothiophene, thiazole, dibenzodioxin or benzothiazole,
the cyclic divalent to tetravalent aliphatic hydrocarbon group is adamantane, diamantane or cyclohexane, and
the cyclic divalent to tetravalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.
   [12]

The material for metal patterning according to [8], wherein
a substituent of L¹²¹ and L¹³¹ is each independently one or more groups selected from the group consisting of
a methyl group, an ethyl group, a methoxy group, an ethoxy group, a trifluoromethyl group, a trifluoromethoxy group, a perfluoroalkyl group with 2 to 10 carbon atoms, a perfluoroalkoxy group with 2 to 10 carbon atoms, a cyano group, a deuterium atom, a fluorine atom, a phenyl group, a biphenylyl group, a naphthyl group, a phenanthryl group, a pyridyl group, a carbazolyl group, a dibenzothienyl group, a dibenzofuranyl group and a structure represented by the formula (001), and
a combination of these groups. [13]

The material for metal patterning according to [3], wherein the monocyclic, linked or condensed divalent to tetravalent aromatic hydrocarbon group represented by L¹¹¹ is a phenyl group or has a structure in which a plurality of benzene rings are linked or condensed. [14]

The material for metal patterning according to [3], wherein the monocyclic, linked or condensed divalent to tetravalent heteroaromatic group represented by L¹¹¹ has N, O or S as a heteroatom and is a 5-membered ring, a 6-membered ring or a condensed structure thereof. [15]

the monocyclic, linked or condensed divalent to tetravalent aromatic hydrocarbon group represented by L¹¹¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene or dibenzochrysene,
the monocyclic, linked or condensed divalent to tetravalent heteroaromatic group is pyridine, pyrimidine, pyrazine, triazine, carbazole, furan, thiophene, benzofuran, benzothiophene, benzodioxin, dibenzofuran, dibenzothiophene, thiazole, dibenzodioxin or benzothiazole,
the cyclic divalent to tetravalent aliphatic hydrocarbon group is adamantane, diamantane or cyclohexane, and
the cyclic divalent to tetravalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane. [16]

The material for metal patterning according to [3], wherein
a substituent of L¹¹¹ is each independently one or more groups selected from the group consisting of
a methyl group, an ethyl group, a methoxy group, an ethoxy group, a trifluoromethyl group, a trifluoromethoxy group, a perfluoroalkyl group with 2 to 10 carbon atoms, a perfluoroalkoxy group with 2 to 10 carbon atoms, a cyano group, a deuterium atom, a fluorine atom, a phenyl group, a biphenylyl group, a naphthyl group, a phenanthryl group, a pyridyl group, a carbazolyl group, a dibenzothienyl group, a dibenzofuranyl group and a structure represented by the formula (001), and
a combination of these groups. [17]

The material for metal patterning according to [8], wherein the monocyclic, linked or condensed monovalent aromatic hydrocarbon group represented by R¹¹¹ or R¹²¹ is a phenyl group or has a structure in which a plurality of benzene rings are linked or condensed. [18]

The material for metal patterning according to [4], wherein the monocyclic, linked or condensed monovalent heteroaromatic group represented by R¹¹¹ or R¹²¹ has N, O or S as a heteroatom and is a 5-membered ring, a 6-membered ring or a condensed structure thereof. [19]

The material for metal patterning according to [4], wherein
the monocyclic, linked or condensed monovalent aromatic hydrocarbon group represented by R¹¹¹ or R¹²¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene or dibenzochrysene,
the monocyclic, linked or condensed monovalent heteroaromatic group is pyridine, pyrimidine, pyrazine, triazine, carbazole, furan, thiophene, benzofuran, benzothiophene, benzodioxin, dibenzofuran, dibenzothiophene, dibenzodioxin, thiazole or benzothiazole,
the linear, branched or cyclic monovalent aliphatic hydrocarbon group is adamantane, diamantane, cyclohexane or methane, and
the cyclic monovalent heteroaliphatic hydrocarbon group is piperazine, homopiperazine, hexahydro-1,3,5-triazine or 1,4-dioxin.

The material for metal patterning according to [4], wherein
a substituent of R¹¹¹ and R¹²¹ is each independently one or more groups selected from the group consisting of
a methyl group, an ethyl group, a methoxy group, an ethoxy group, a trifluoromethyl group, a trifluoromethoxy group, a perfluoroalkyl group with 2 to 10 carbon atoms, a perfluoroalkoxy group with 2 to 10 carbon atoms, a cyano group, a deuterium atom, a fluorine atom, a phenyl group, a biphenylyl group, a naphthyl group, a phenanthryl group, a pyridyl group, a carbazolyl group, a dibenzothienyl group, a dibenzofuranyl group and a structure represented by the formula (001), and
a combination of these groups.
   [21]

A heterocyclic compound represented by the formula (301) or (302):

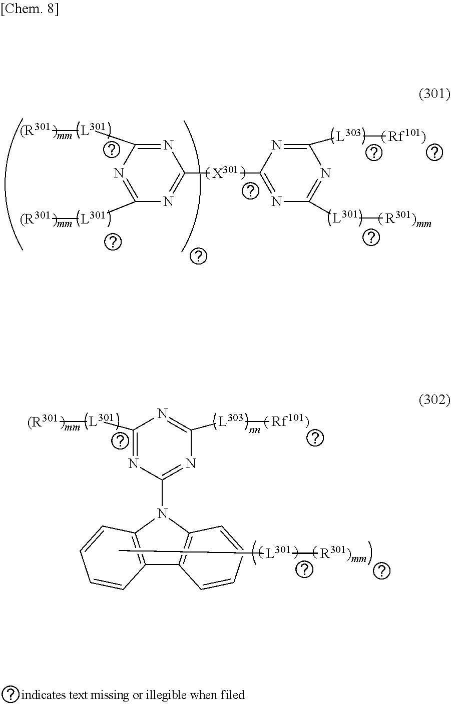

L³⁰¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O, S or NR³⁰¹;
L³⁰³ each independently denotes
a linear, branched or cyclic divalent to tetravalent aliphatic hydrocarbon group with 1 to 18 carbon atoms optionally substituted with fluorine, or
or S;
Rf³⁰¹ each independently denotes
a moiety with 2 or more carbon atoms containing 3 or more fluorine atoms;
R³⁰¹ each independently denotes
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted cyclic monovalent aliphatic hydrocarbon group with 3 to 18 carbon atoms, or
a fluoroalkyl structure with 1 or more carbon atoms containing 3 or more fluorine atoms;
X³⁰¹ denotes
an optionally substituted monocyclic, linked or condensed divalent to tetravalent aromatic hydrocarbon group with 3 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or O; and
gg each independently denotes an integer in the range of 0 to 6.
hh each independently denotes an integer in the range of 0 to 12.
ii each independently denotes an integer in the range of 1 to 6.
jj each independently denotes an integer in the range of 0 to 6.
kk each independently denotes an integer in the range of 1 to 6.
mm each independently denotes an integer in the range of 1 to 6.
nn each independently denotes an integer in the range of 1 to 6.

In the formula (301), when L³⁰¹ is NR³⁰¹, and R³⁰¹ is an optionally substituted monocyclic, linked or condensed monovalent aromatic hydrocarbon group with 6 to 26 carbon atoms, L³⁰¹ is each independently substituted with a fluorine atom or a moiety with 1 or more carbon atoms containing 3 or more fluorine atoms.

When X³⁰¹ is a p-phenylene group and nn is 1, the p-phenylene group is substituted with a fluorine atom or a monovalent substituent with 1 or more carbon atoms optionally containing 3 or more fluorine atoms.

X³⁰¹ does not have a cyano group, a linear vinylene group, a ketone group, a thioketone group or a selenium atom.

[22]

The heterocyclic compound according to [21], wherein Rf³⁰¹ is represented by the following formula (311):

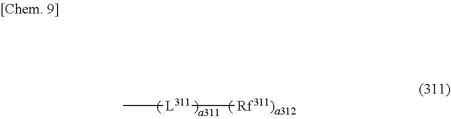

wherein
Rf³¹¹ each independently denotes
a linear, branched or cyclic aliphatic hydrocarbon group with 1 to 18 carbon atoms optionally substituted with a fluorine atom;
L³¹¹ each independently denotes
a linear, branched or cyclic divalent aliphatic hydrocarbon group with 1 to 18 carbon atoms optionally substituted with O, S or a fluorine atom, or
a vinylene group that may form a ring;
a311 each independently denotes an integer in the range of 0 to 4; and
a312 each independently denotes an integer in the range of 1 to 4.
   [23]

A thin film for metal patterning containing a material for metal patterning and capable of patterning a metal film or a metal multilayer film, wherein
the material for metal patterning contains a compound represented by the formula (A1) or (B1) according to [1] or the formula (101), (102), (103), (104) or (105) according to [4], and
the metal film or the metal multilayer film contains at least one selected from the group consisting of ytterbium, magnesium, silver, aluminum and an alloy of magnesium and silver.
   [24]

A thin film for metal patterning containing the material for metal patterning according to any one of [1] to [20], wherein a water contact angle is 90 degrees or more.

[25]

An organic electroluminescent element having a negative electrode, wherein
the negative electrode
contains at least one selected from the group consisting of ytterbium, magnesium, silver, aluminum and an alloy of magnesium and silver, and
is patterned with a material for metal patterning, and
the material for metal patterning contains a compound represented by the formula (A1) or (B1) according to [1] or the formula (101), (102), (103), (104) or (105) according to [4].
   [26]

A method for forming a metal pattern, including
forming an organic material pattern containing the material for metal patterning according to any one of [1] to [20]; and
applying a metallic material to a region where the organic material pattern is formed and a region where the organic material pattern is not formed to form a metal pattern in the region where the organic material pattern is not formed.
   [27]

An electronic device containing the material for metal patterning according to any one of [1] to [20].

### Advantageous Effects of Invention

One aspect of the present disclosure can provide a material for metal patterning capable of significantly suppressing the formation of a thin metal film on a film surface, a thin film for metal patterning using the material for metal patterning, an organic electroluminescent element, a method for forming a metal pattern, an electronic device and a heterocyclic compound.

### BRIEF DESCRIPTION OF DRAWINGS

FIG. 1A is a graph of the transmittance of light through a compound Z1 before metal evaporation.

FIG. 1B is a graph of the transmittance of light through the compound Z1 after metal evaporation.

FIG. 2A is a graph of the transmittance of light through a compound Z190 before metal evaporation.

FIG. 2B is a graph of the transmittance of light through the compound Z190 after metal evaporation.

FIG. 3A is a graph of the transmittance of light through a compound X1 before metal evaporation.

FIG. 3B is a graph of the transmittance of light through the compound X1 after metal evaporation.

FIG. 4A is a graph of the transmittance of light through a compound X6 before metal evaporation.

FIG. 4B is a graph of the transmittance of light through the compound X6 after metal evaporation.

### DESCRIPTION OF EMBODIMENTS

A material for metal patterning, an electronic device and a heterocyclic compound, as well as a thin film for metal patterning, an organic electroluminescent element and a method for forming a metal pattern using these according to an embodiment of the present disclosure are described in detail below.

### First Embodiment: Material for Metal Patterning

A material for metal patterning according to an embodiment of the present disclosure is the following material containing a compound represented by the formula (A1) or (B1).

A material for metal patterning represented by the formula (A1) or (B1):

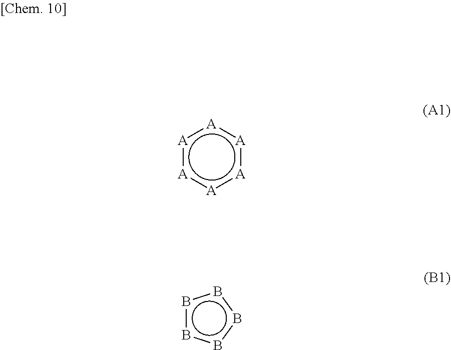

wherein
A each independently denotes N or CR, at least one thereof being N and at least one thereof being CR;
B each independently denotes N, S, O or CR, at least one thereof being N, S or O and at least one thereof being CR;
R each independently denotes the formula (A01) or the formula (A02), at least one thereof being the formula (A01):

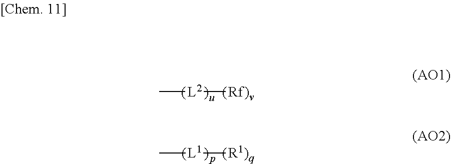

L¹ and L² each independently denote
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR²;
Rf each independently denotes
a moiety with 1 or more carbon atoms containing 3 or more fluorine atoms;
R¹ and R² each independently denote
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
   an optionally substituted cyclic monovalent heteroaliphatic hydrocarbon group with 3 to 18 carbon atoms, or
H, D (deuterium atom) or F (fluorine atom); and
p each independently denotes an integer in the range of 0 to 12.

q each independently denotes an integer in the range of 1 to 6.

u each independently denotes an integer in the range of 0 to 12.

v each independently denotes an integer in the range of 1 to 6.

When L¹ and L² are a substituted divalent to tetravalent aromatic hydrocarbon group, a substituted divalent to tetravalent heteroaromatic group, a substituted divalent to tetravalent aliphatic hydrocarbon group or a substituted divalent to tetravalent heteroaliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a cyano group, a fluorine atom and a deuterium atom,
a structure represented by the formula (001), and
a combination of these groups;
when R¹ and R² are a substituted monovalent aromatic hydrocarbon group, a substituted monovalent heteroaromatic group, a substituted monovalent aliphatic hydrocarbon group or a substituted monovalent heteroaliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a cyano group, a fluorine atom and a deuterium atom,
a structure represented by the formula (001), and
a combination of these groups.
   (As for the formula (A1) and the formula (B1))

A each independently denotes N or CR, at least one thereof being N and at least one thereof being CR; and
as the formula (A1), for example, pyridine, pyridazine, pyrimidine, pyrazine or triazine can be formed.

B each independently denotes N, NR, O, S or CR, at least one thereof being N, NR, O or S and at least one thereof being CR; and
as the formula (B1), for example, pyrrole, furan, thiophene, imidazole, oxazole, thiazole, pyrazole, isoxazole, isothiazole, oxadiazole, thiadiazole, triazole or tetrazole can be formed.

Rs bound to the formula (A1) or the formula (B1) may be bound together and form a ring.

When Rs bound to the formula (A1) are bound together and form a ring, for example, quinoline, isoquinoline, quinazoline, quinoxaline, phenanthridine, acridine, phenanthroline, azatriphenylene, azaindole or the like can be formed.

When Rs bound to the formula (B1) are bound together and form a ring, for example, benzofuran, benzothiophene, benzimidazole, benzothiazole, benzoxazole, dibenzofuran, dibenzothiophene or the like can be formed.

Pyridine, pyrimidine, pyrazine or triazine is preferred as the formula (A1), and thiazole is preferred as the formula (B1).

In the formula (A01), Rf denotes a group having, as a partial structure, at least one structure of a fluoroalkyl group, a fluoroalkoxy group, a fluoroalkenyl group or a fluoroalkenyloxy group in which one or more hydrogen atoms in the alkyl group, the alkoxy group, the alkenyl group or the alkenyloxy group are substituted with a fluorine atom.

Rf contains 3 or more fluorine atoms and 1 or more carbon atoms. Furthermore, the ratio of the number of carbon atoms directly bound to a fluorine atom to the carbon atoms forming Rf is preferably 30% or more. The ratio is more preferably 40% or more, still more preferably 50% or more, still more preferably 60% or more, particularly preferably 70% or more.

The molecular structure represented by Rf is not particularly limited and may be linear, branched or cyclic.

The molecular structure giving Rf may be a linear, branched or cyclic alkyl group or a linear, branched or cyclic alkenyl group. The cyclic alkyl group or the cyclic alkenyl group includes those in which a hydrogen atom bound to a ring carbon atom is substituted with an alkyl group or an alkenyl group. The number of carbon atoms in the alkyl group or the alkenyl group bound to the ring carbon atom is preferably 1 or more and 6 or less.

The cyclic alkyl group or the cyclic alkenyl group may be a monovalent group formed when one hydrogen atom is removed from a ring carbon atom or may be a monovalent group formed when one hydrogen atom is removed from the alkyl group or the alkenyl group bound to the ring carbon atom (that is, a cycloalkylalkyl group, a cycloalkylalkenyl group, a cycloalkenylalkyl group or a cycloalkenylalkenyl group).

An alkyl group that provides the fluoroalkyl group is, for example, a methyl group, an ethyl group, a n-propyl group, a n-butyl group, a n-pentyl group, a n-hexyl group, a n-heptyl group, a n-octyl group, an alkyl group having structural isomerism with these alkyl groups, a cyclopropyl group, a cyclobutyl group, a cyclopentyl group, a 1-methylcyclopentyl group, a cyclopentylmethyl group, a cyclohexyl group, an adamantyl group or the like. Among these groups, from the perspective of high metal patterning performance, a methyl group, an ethyl group, a n-propyl group, a n-butyl group, a n-pentyl group, a n-hexyl group, an alkyl group having structural isomerism with these alkyl groups and a cyclohexyl group are preferred.

An alkoxy group that provides the fluoroalkoxy group may be an alkoxy group in which an alkyl group that provides the fluoroalkyl group is bound to an oxygen atom. Among such alkoxy groups, from the perspective of high metal patterning performance, an alkoxy group in which a methyl group, an ethyl group, a n-propyl group, a n-butyl group, a n-pentyl group, a n-hexyl group or an alkyl group having structural isomerism with these alkyl groups is bound to an oxygen atom is preferred.

An alkenyl group that provides the fluoroalkenyl group is, for example, a vinyl group, a 1-propenyl group, a 1-butenyl group, a 1-pentynyl group, a 1-hexenyl group, an alkenyl group having structural isomerism with these alkenyl groups, a 1-cyclopropenyl group, a 1-cyclobutenyl group, a 1-cyclopentynyl group, a 1-cyclohexyl group, a cycloalkenyl group having structural isomerism with these cycloalkenyl groups or the like. Among these groups, from the perspective of high metal patterning performance, a vinyl group, a 1-propenyl group, a 1-butenyl group, a 1-pentynyl group, a 1-hexenyl group, an alkenyl group having structural isomerism with these alkenyl groups, a 1-cyclopentynyl group and a 1-cyclohexyl group are preferred.

An alkenyloxy that provides the fluoroalkenyloxy group may be an alkenyloxy group in which an alkenyl group that provides the fluoroalkenyl group is bound to an oxygen atom. Among such alkenyloxy groups, from the perspective of high metal patterning performance, an alkenyloxy group in which a vinyl group, a 1-propenyl group, a 1-butenyl group, a 1-pentynyl group, a 1-hexenyl group, an alkenyl group having structural isomerism with these alkenyl groups, a 1-cyclopentynyl group or a 1-cyclohexyl group is bound to an oxygen atom is preferred.

Rf preferably has a structure represented by the following formula (001):

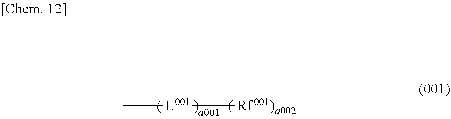

wherein
Rf⁰⁰¹ each independently denotes
a linear, branched or cyclic aliphatic hydrocarbon group with 1 to 18 carbon atoms containing 3 or more fluorine atoms;
L⁰⁰¹ each independently denotes
a linear, branched or cyclic divalent aliphatic hydrocarbon group with 1 to 18 carbon atoms optionally substituted with O, S or a fluorine atom, or
a vinylene group that may be substituted with fluorine and may form a ring;
a001 each independently denotes an integer in the range of 0 to 4; and
a002 each independently denotes an integer in the range of 1 to 4.

In each Rf⁰⁰¹, the ratio of the number of carbon atoms directly bound to a fluorine atom to the number of carbon atoms constituting the structure is preferably 30% or more, more preferably 40% or more, still more preferably 50% or more, still more preferably 60% or more, particularly preferably 70% or more.

Rf⁰⁰¹ preferably each independently denotes a methyl group, an ethyl group, a n-propyl group, a n-butyl group, a n-pentyl group, a n-hexyl group, a n-heptyl group, a n-octyl group, an alkyl group having structural isomerism with these alkyl groups, a cyclopropyl group, a cyclobutyl group, a cyclopentyl group, a 1-methylcyclopentyl group, a cyclopentylmethyl group, a cyclohexyl group or an adamantyl group, each containing 3 or more fluorine atoms.

From the perspective of being able to suppress the formation of a metal film on a film surface, L⁰⁰¹ more preferably each independently denotes
O, S or
a methylene group, an ethylene group, a n-propylene group, a n-butylene group, a n-pentylene group, a n-hexylene group, a n-heptylene group, a n-octylene group, an alkylene group having structural isomerism with these alkylene groups, a cyclopropylene group, a cyclobutylene group, a cyclopentylene group, a 1-methylpentylene group, a cyclopentylmethyl group, a cyclohexylene group or an adamantanediyl group, each of which may be substituted with a fluorine atom, or
a vinylene group that may form a ring.

a001 preferably each independently ranges from 0 to 3, more preferably 0 to 2.

a002 preferably each independently ranges from 1 to 3, more preferably 1 to 2.

The number of carbon atoms in the structure represented by the formula (001) is preferably 1 or more and 36 or less, more preferably 1 or more and 30 or less, still more preferably 1 or more and 24 or less, still more preferably 1 or more and 20 or less, particularly preferably 1 or more and 16 or less. 2 or more and 16 or less is particularly preferred.

### Specific Examples of Rf

Rf may have a structure, for example, represented by one of the following (AAA1) to (AAA126).

The mark “*” in the following structures denotes a binding site.

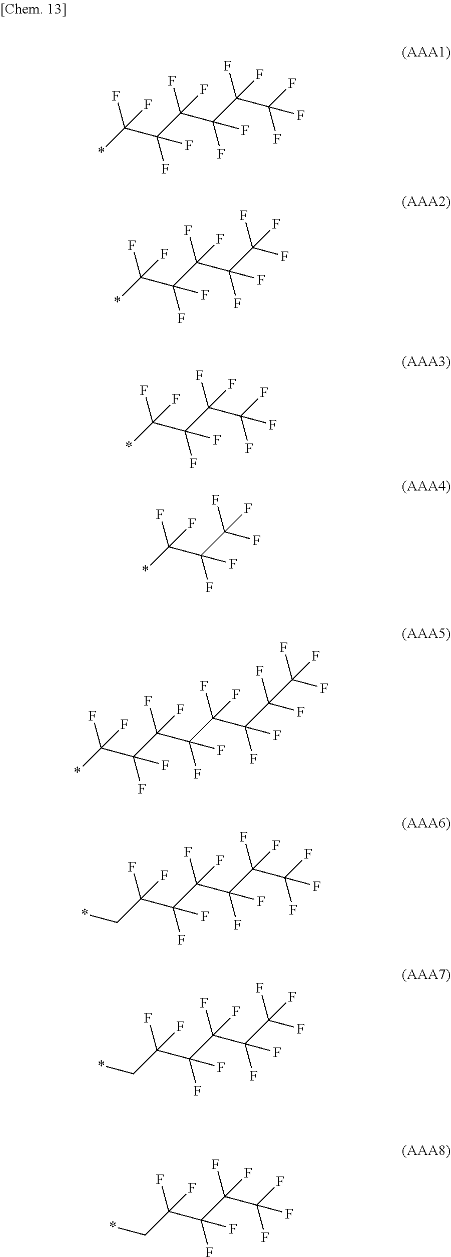

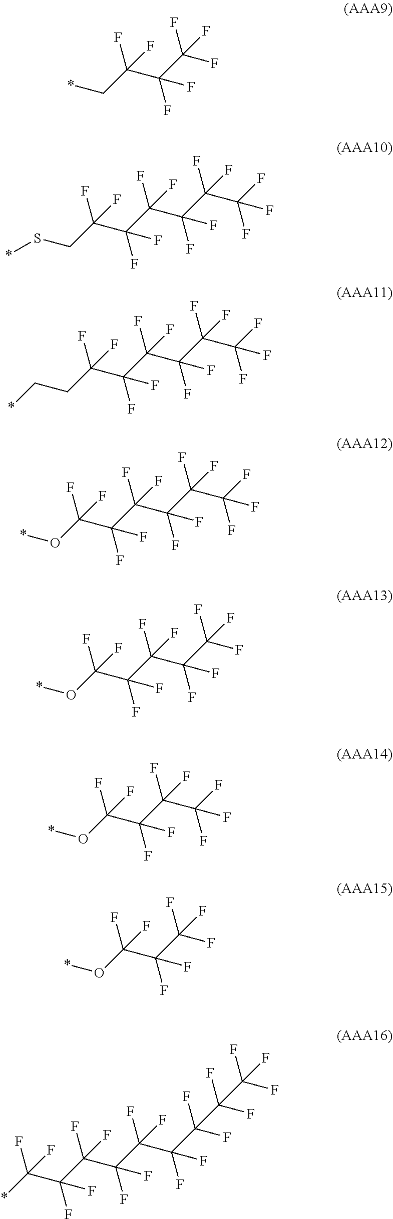

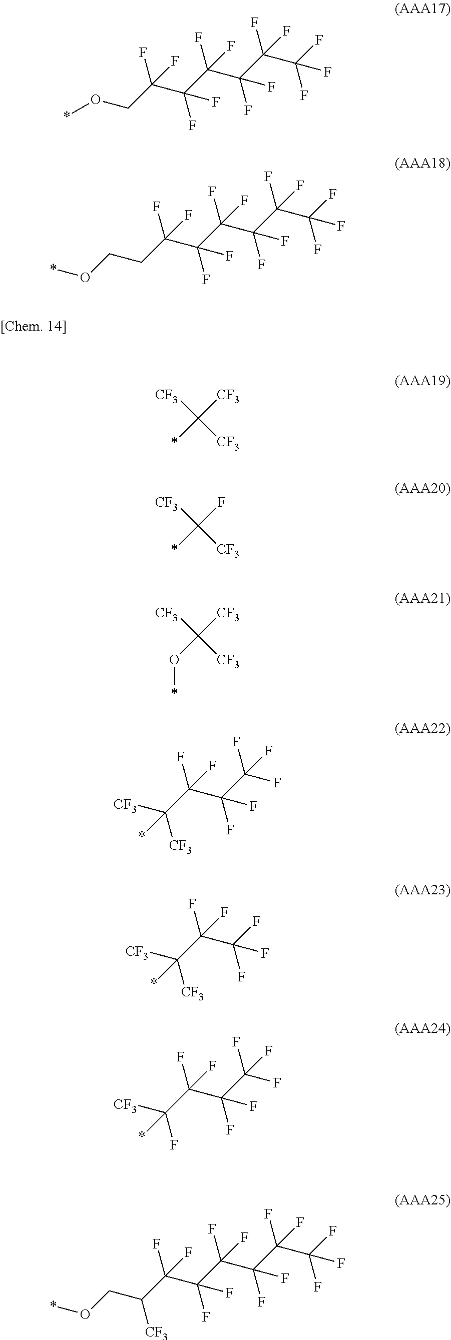

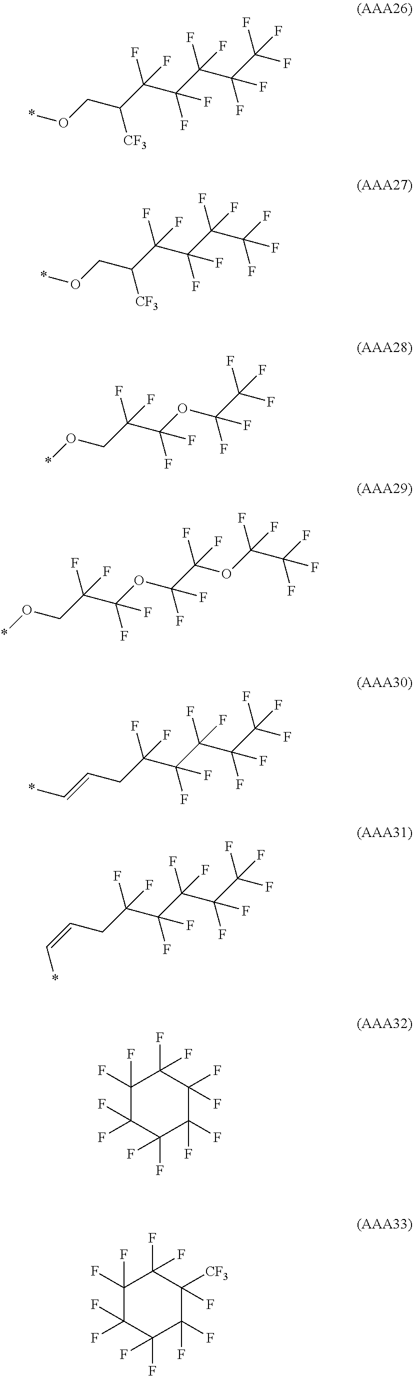

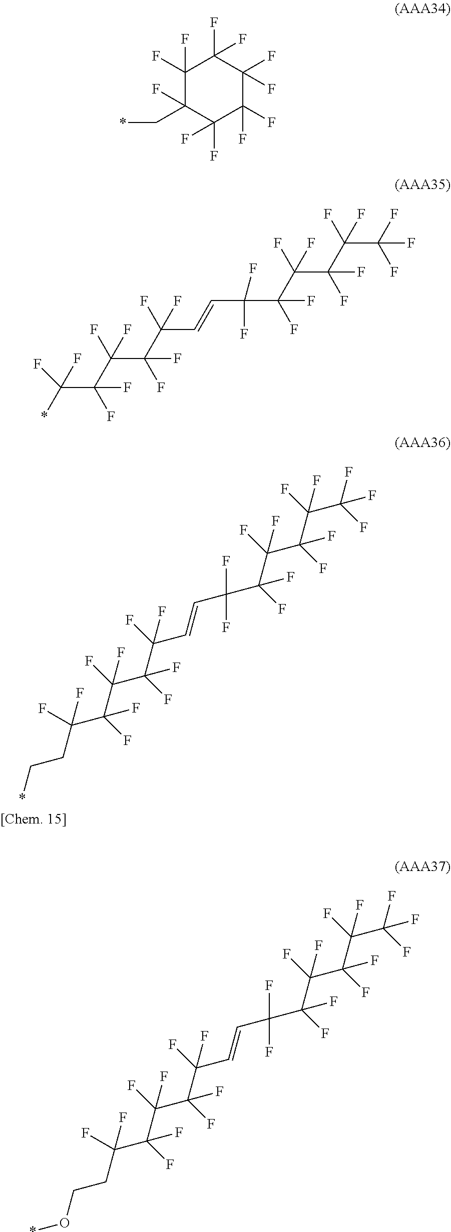 [SMILES: *CC1(F)C(F)(F)C(F)(F)C(F)(F)C(F)(F)C1(F)F ; *C(F)(F)C(F)(F)C(F)(F)C(F)(F)C(F)(F)/C=C/C(F)(F)C(F)(F)C(F)(F)C(F)(F)C(F)(F)C(F)(F)F ; *CCC(F)(F)C(F)(F)C(F)(F)C(F)(F)C(F)(F)/C=C/C(F)(F)C(F)(F)C(F)(F)C(F)(F)C(F)(F)C(F)(F)F ; *OCCC(F)(F)C(F)(F)C(F)(F)C(F)(F)C(F)(F)/C=C/C(F)(F)C(F)(F)C(F)(F)C(F)(F)C(F)(F)C(F)(F)F]

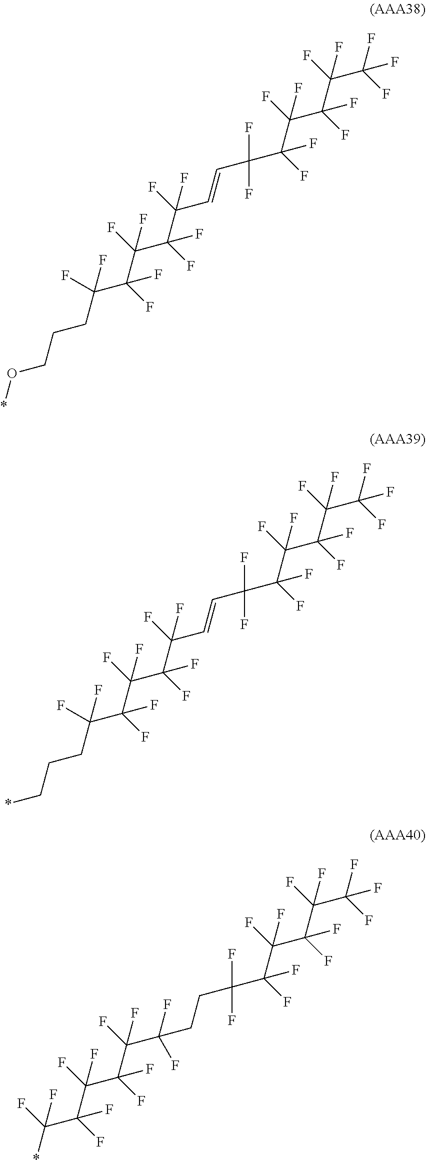 [SMILES: *OCCCC(F)(F)C(F)(F)C(F)(F)C(F)(F)C(F)(F)/C=C/C(F)(F)C(F)(F)C(F)(F)C(F)(F)C(F)(F)C(F)(F)F ; *CCCC(F)(F)C(F)(F)C(F)(F)C(F)(F)C(F)(F)/C=C/C(F)(F)C(F)(F)C(F)(F)C(F)(F)C(F)(F)C(F)(F)F ; *C(F)(F)C(F)(F)C(F)(F)C(F)(F)C(F)(F)C(F)(F)CCC(F)(F)C(F)(F)C(F)(F)C(F)(F)C(F)(F)C(F)(F)F]

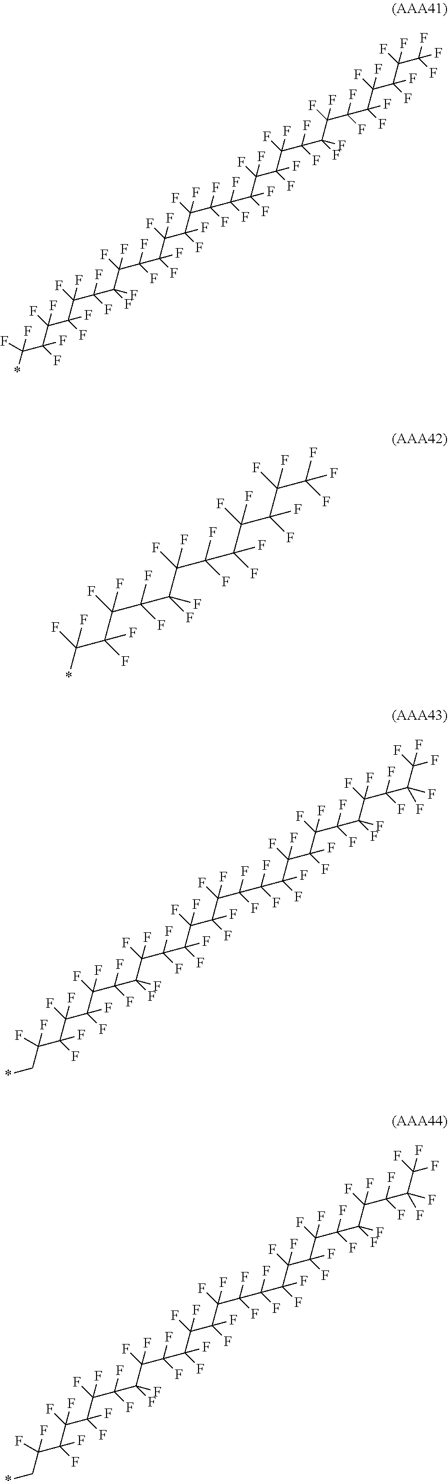

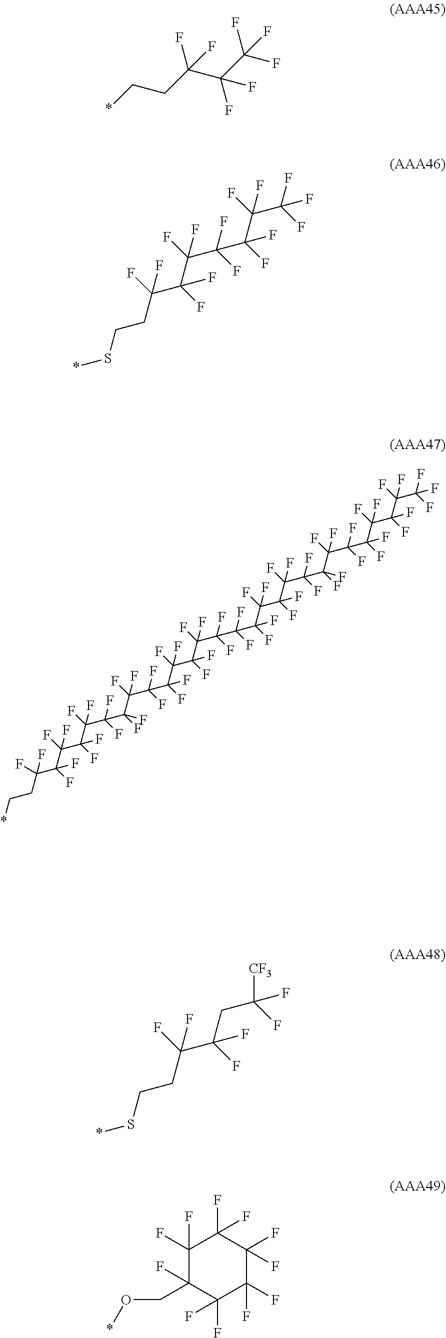 [SMILES: *CCC(F)(F)C(F)(F)C(F)(F)F ; *SCCC(F)(F)C(F)(F)C(F)(F)C(F)(F)C(F)(F)C(F)(F)C(F)(F)F ; *CCC(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)F ; *SCCC(F)(F)C(F)(F)CC(F)(F)C(F)(F)F ; *OCC1(F)C(F)(F)C(F)(F)C(F)(F)C(F)(F)C1(F)F]

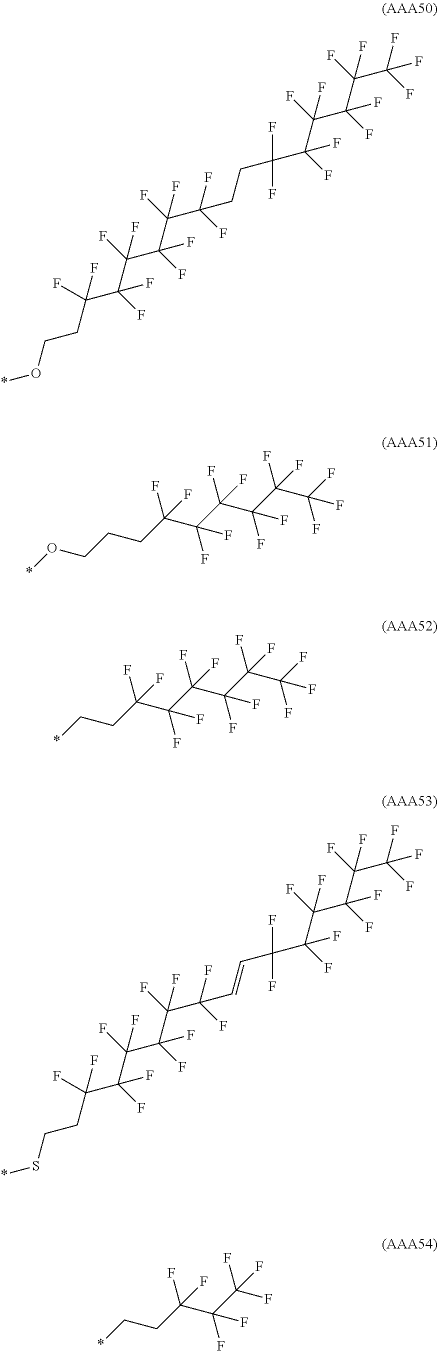 [SMILES: *OCCC(F)(F)C(F)(F)C(F)(F)C(F)(F)C(F)(F)C(F)(F)CCC(F)(F)C(F)(F)C(F)(F)C(F)(F)C(F)(F)C(F)(F)F ; *OCCCC(F)(F)C(F)(F)C(F)(F)C(F)(F)C(F)(F)C(F)(F)F ; *CCC(F)(F)C(F)(F)C(F)(F)C(F)(F)C(F)(F)C(F)(F)F ; *SCCC(F)(F)C(F)(F)C(F)(F)C(F)(F)C(F)(F)C(F)(F)/C=C/C(F)(F)C(F)(F)C(F)(F)C(F)(F)C(F)(F)C(F)(F)F ; *CCC(F)(F)C(F)(F)C(F)(F)F]

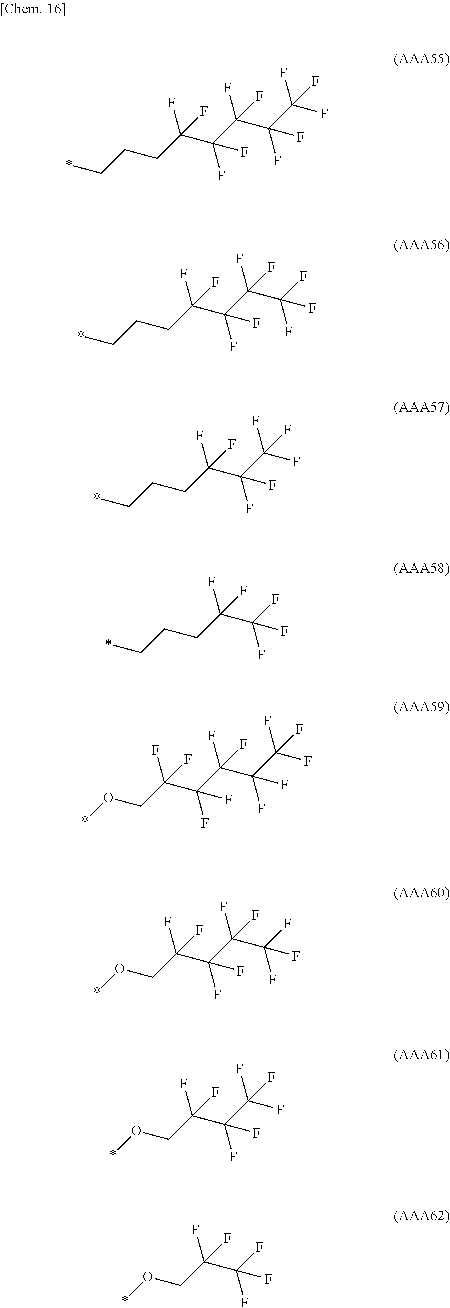

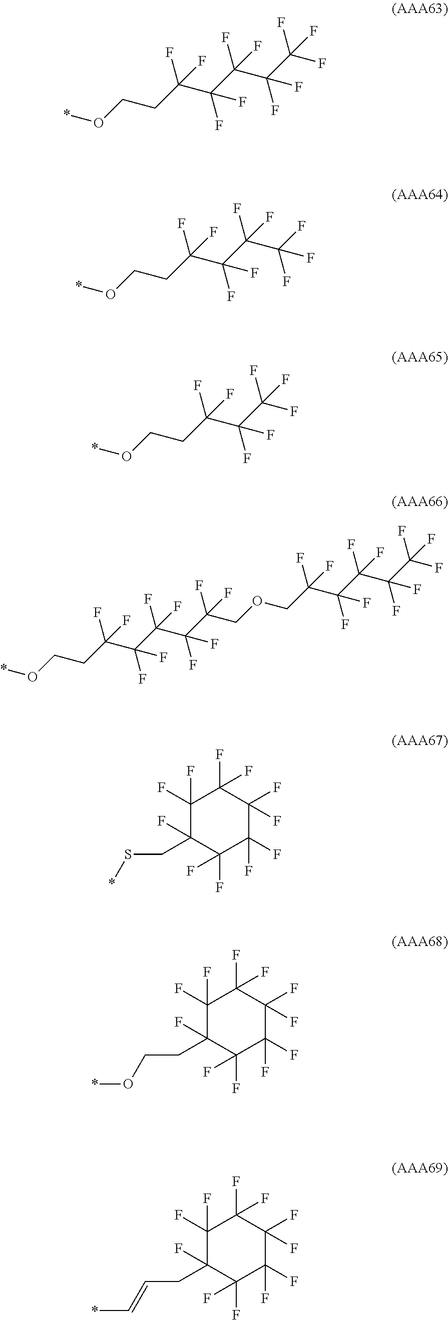

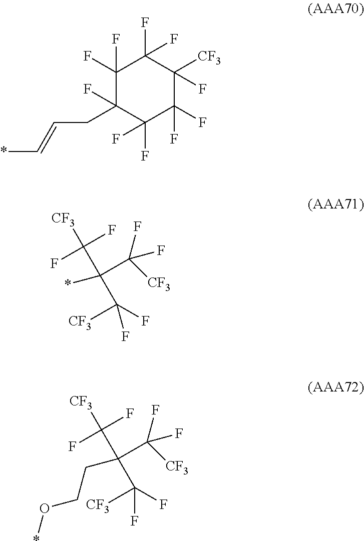 [SMILES: */C=C/CC1(F)C(F)(F)C(F)(F)C(F)(C(F)(F)F)C(F)(F)C1(F)F ; *C(C(F)(F)C(F)(F)F)(C(F)(F)C(F)(F)F)C(F)(F)C(F)(F)F ; *OCCC(C(F)(F)C(F)(F)F)(C(F)(F)C(F)(F)F)C(F)(F)C(F)(F)F]

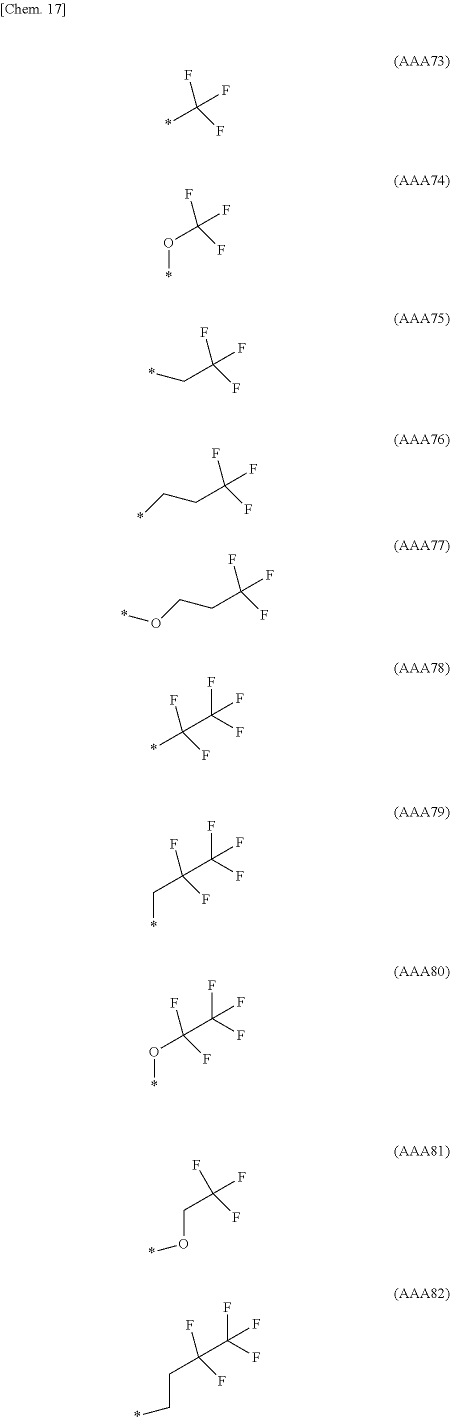

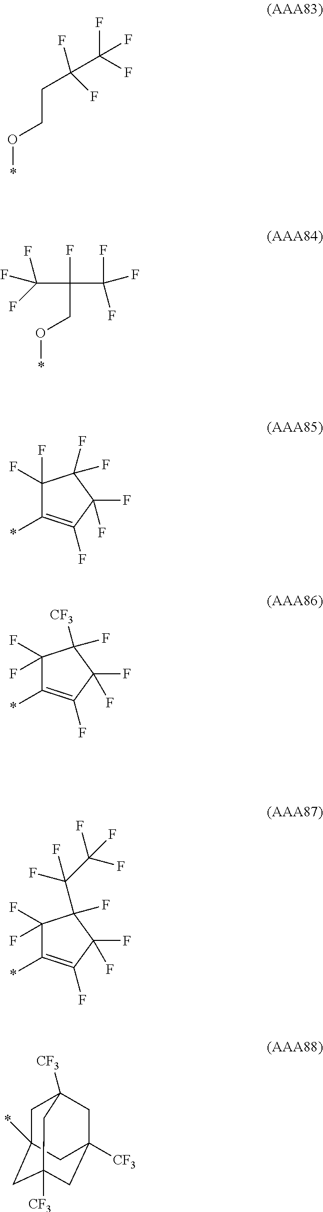 [SMILES: *OCCC(F)(F)C(F)(F)F ; *OCC(F)(C(F)(F)F)C(F)(F)F ; *C1=C(F)C(F)(F)C(F)(F)C1(F)F ; *C1=C(F)C(F)(F)C(F)(C(F)(F)F)C1(F)F ; *C1=C(F)C(F)(F)C(F)(C(F)(F)C(F)(F)F)C1(F)F ; *C12CC3(C(F)(F)F)CC(C(F)(F)F)(C1)CC(C(F)(F)F)(C2)C3]

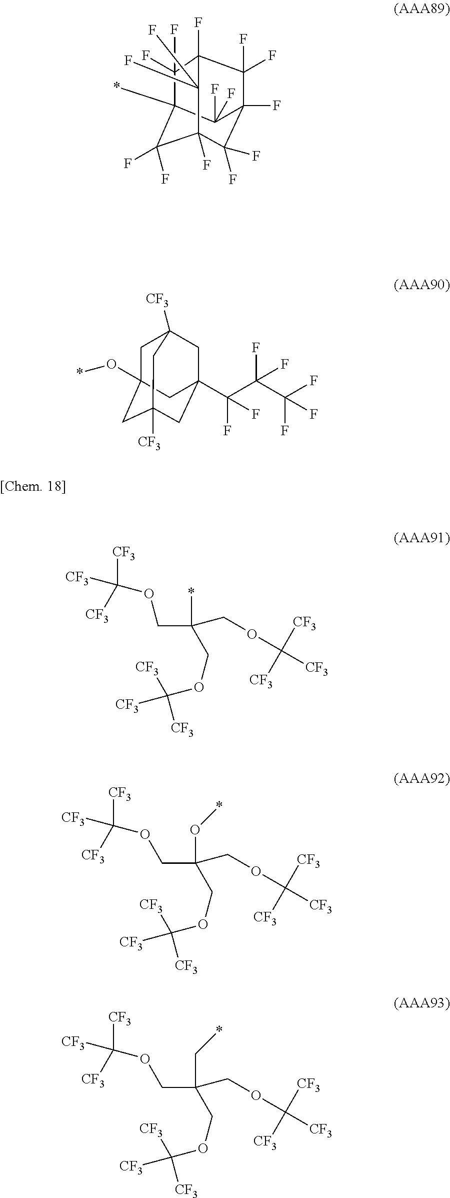 [SMILES: *C12C(F)(F)C3(F)C(F)(F)C(F)(C1(F)F)C(F)(F)C(F)(C2(F)F)C3(F)F ; *OC12CC3(C(F)(F)F)CC(C(F)(F)F)(C1)CC(C(F)(F)C(F)(F)C(F)(F)F)(C2)C3 ; *C(COC(C(F)(F)F)(C(F)(F)F)C(F)(F)F)(COC(C(F)(F)F)(C(F)(F)F)C(F)(F)F)COC(C(F)(F)F)(C(F)(F)F)C(F)(F)F ; *OC(COC(C(F)(F)F)(C(F)(F)F)C(F)(F)F)(COC(C(F)(F)F)(C(F)(F)F)C(F)(F)F)COC(C(F)(F)F)(C(F)(F)F)C(F)(F)F ; *CC(COC(C(F)(F)F)(C(F)(F)F)C(F)(F)F)(COC(C(F)(F)F)(C(F)(F)F)C(F)(F)F)COC(C(F)(F)F)(C(F)(F)F)C(F)(F)F]

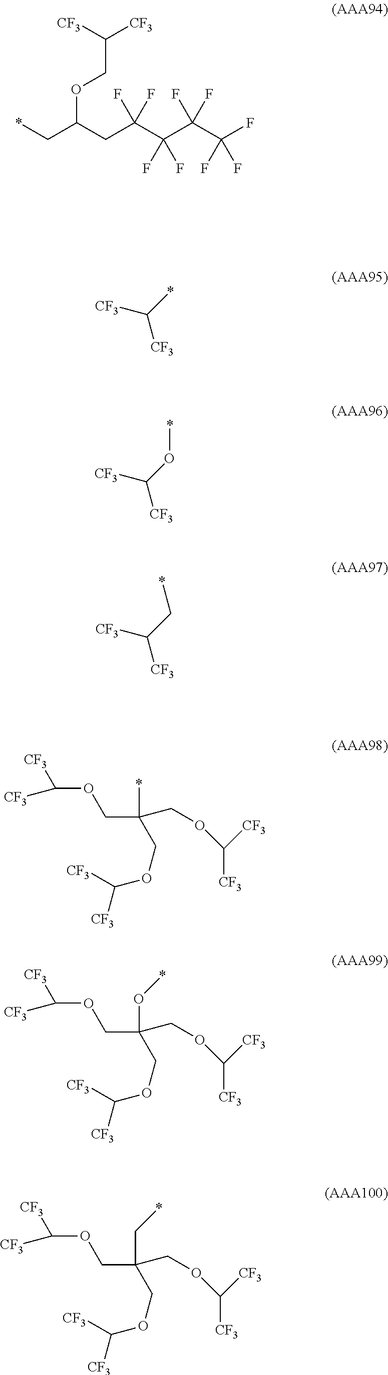

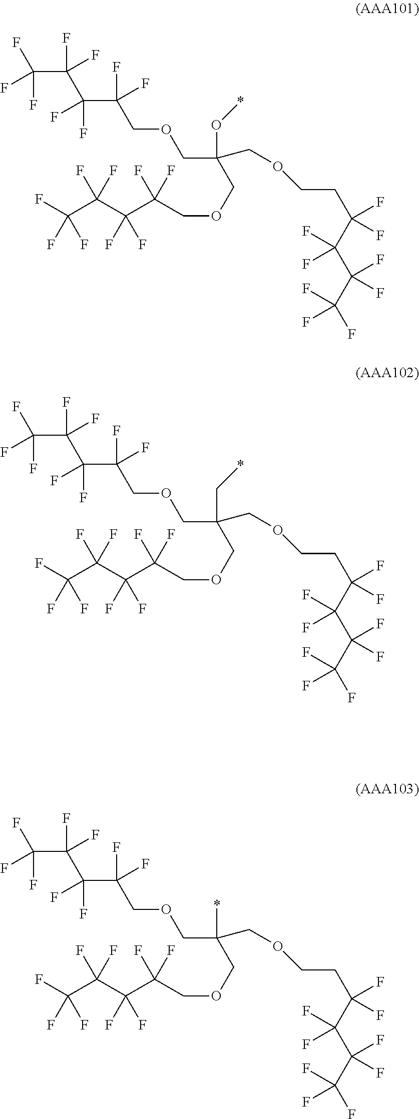 [SMILES: *OC(COCCC(F)(F)C(F)(F)C(F)(F)C(F)(F)F)(COCC(F)(F)C(F)(F)C(F)(F)C(F)(F)F)COCC(F)(F)C(F)(F)C(F)(F)C(F)(F)F ; *CC(COCCC(F)(F)C(F)(F)C(F)(F)C(F)(F)F)(COCC(F)(F)C(F)(F)C(F)(F)C(F)(F)F)COCC(F)(F)C(F)(F)C(F)(F)C(F)(F)F ; *C(COCCC(F)(F)C(F)(F)C(F)(F)C(F)(F)F)(COCC(F)(F)C(F)(F)C(F)(F)C(F)(F)F)COCC(F)(F)C(F)(F)C(F)(F)C(F)(F)F]

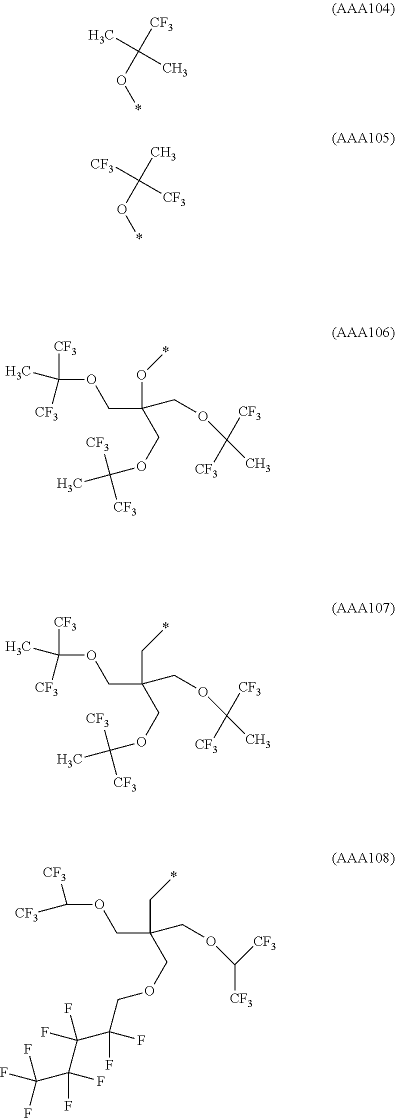

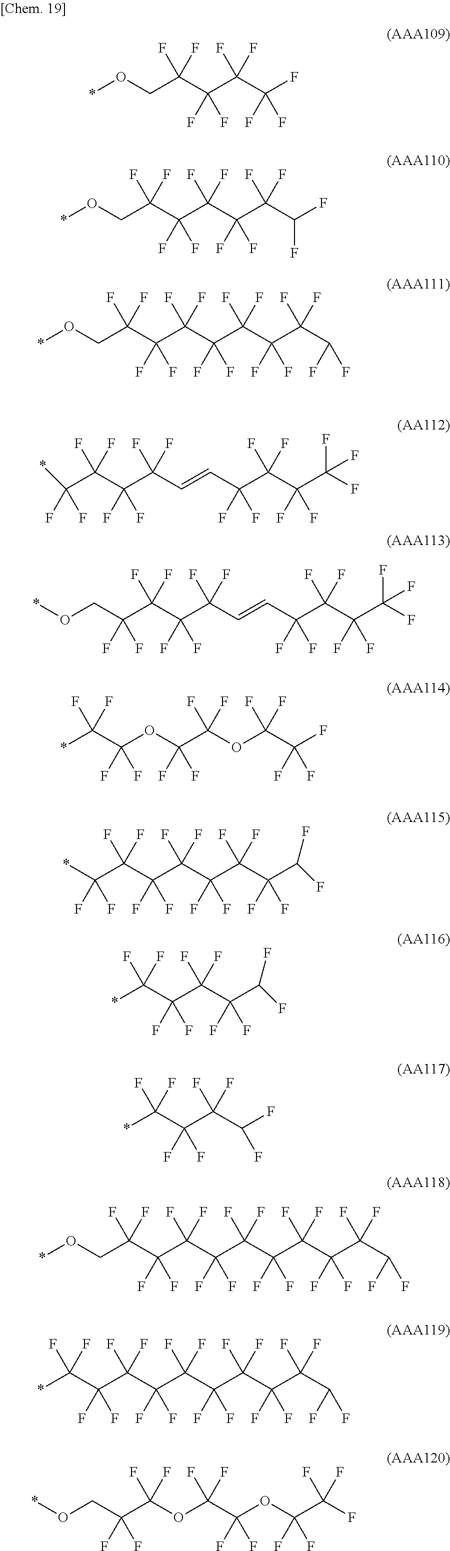

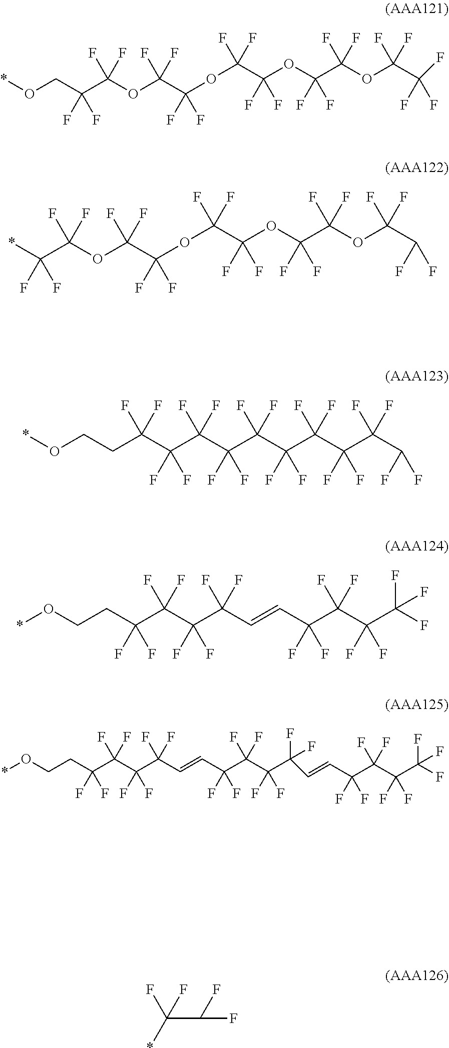 [SMILES: *OCC(F)(F)C(F)(F)OC(F)(F)C(F)(F)OC(F)(F)C(F)(F)OC(F)(F)C(F)(F)OC(F)(F)C(F)(F)F ; *C(F)(F)C(F)(F)OC(F)(F)C(F)(F)OC(F)(F)C(F)(F)OC(F)(F)C(F)(F)OC(F)(F)C(F)F ; *OCCC(F)(F)C(F)(F)C(F)(F)C(F)(F)C(F)(F)C(F)(F)C(F)(F)C(F)(F)C(F)(F)C(F)F ; *OCCC(F)(F)C(F)(F)C(F)(F)C(F)(F)/C=C/C(F)(F)C(F)(F)C(F)(F)C(F)(F)F ; *OCCC(F)(F)C(F)(F)C(F)(F)C(F)(F)/C=C/C(F)(F)C(F)(F)C(F)(F)C(F)(F)/C=C/C(F)(F)C(F)(F)C(F)(F)C(F)(F)F ; *C(F)(F)C(F)F]

(L¹, L²)

L¹ and L² each independently denote
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR².

Preferably, the monocyclic, linked or condensed divalent to tetravalent aromatic hydrocarbon group with 6 to 26 carbon atoms represented by L¹ or L² is benzene or has a structure in which a plurality of benzene rings are linked or condensed.

The monocyclic, linked or condensed divalent to tetravalent aromatic hydrocarbon group with 6 to 26 carbon atoms represented by L¹ or L² is more preferably benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene.

Preferably, the monocyclic, linked or condensed divalent to tetravalent heteroaromatic group with 3 to 26 carbon atoms represented by L¹ or L² has N, O or S as a heteroatom and is a 5-membered ring, a 6-membered ring or a structure in which these or a plurality of benzene rings are condensed.

The monocyclic, linked or condensed divalent to tetravalent heteroaromatic group with 3 to 26 carbon atoms represented by L¹ or L² is more preferably pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene.

A compound that provides the linear, branched or cyclic divalent to tetravalent aliphatic hydrocarbon group with 1 to 18 carbon atoms represented by L¹ or L² is more preferably methane, ethane, propane, butane, adamantane, diamantane, norbornene or cyclohexane.

More preferably, the cyclic divalent to tetravalent heteroaliphatic hydrocarbon group with 3 to 18 carbon atoms represented by L¹ or L² has N, O or S as a heteroatom and is a 5-membered ring, a 6-membered ring or a condensed structure thereof.

A compound that provides the cyclic divalent to tetravalent heteroaliphatic hydrocarbon group with 3 to 18 carbon atoms represented by L¹ or L² is more preferably morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

(R¹, R²)

R¹ and R² each independently denote
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 3 to 18 carbon atoms, or
H, D (deuterium atom) or F (fluorine atom).

Preferably, the monocyclic, linked or condensed monovalent aromatic hydrocarbon group with 6 to 26 carbon atoms represented by R¹ or R² is benzene or has a structure in which a plurality of benzene rings are linked or condensed.

A compound that provides the monocyclic, linked or condensed monovalent aromatic hydrocarbon group with 6 to 26 carbon atoms represented by R¹ or R² is more preferably benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene.

Preferably, the monocyclic, linked or condensed monovalent heteroaromatic group with 3 to 26 carbon atoms represented by R¹ or R² has N, O or S as a heteroatom and is a 5-membered ring, a 6-membered ring or a structure in which these or a plurality of benzene rings are condensed.

A compound that provides the monocyclic, linked or condensed monovalent heteroaromatic group with 3 to 26 carbon atoms represented by R¹ or R² is more preferably pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene.

A compound that provides the linear, branched or cyclic aliphatic hydrocarbon group represented by R¹ or R² is more preferably methane, ethane, propane, butane, pentane, hexane, heptane, octane, nonane, decane, adamantane, diamantane, norbornene, cyclohexane or one of these groups substituted or cyclized with one or more selected from the group consisting of ethane, propane, butane and pentane.

Preferably, the cyclic monovalent heteroaliphatic hydrocarbon group represented by R¹ or R² has N, O or S as a heteroatom and is a 5-membered ring, a 6-membered ring or a condensed structure thereof.

A compound that provides the cyclic monovalent heteroaliphatic hydrocarbon group represented by R¹ or R² is more preferably morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

When R¹ and R² are a substituted monovalent aromatic hydrocarbon group, a substituted monovalent heteroaromatic group or a substituted monovalent aliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a cyano group, a fluorine atom and a deuterium atom,
a structure represented by the formula (001), and
a combination of these groups.

At least one of Rf is preferably directly bound to the formula (A1). Thus, u in at least one formula (A01) in the formula (A1) is preferably 0.

At least one of Rf is preferably directly bound to the formula (B1). Thus, u in at least one formula (A01) in the formula (B1) is preferably 0.

Pyridine, pyrimidine, pyrazine or triazine is preferred as the formula (A1), and thiazole is preferred as the formula (B1).

### Preferred Embodiment 1

A more preferred embodiment of the formula (A1) or the formula (B1) is represented by the formula (101), (102), (103), (104) or (105):

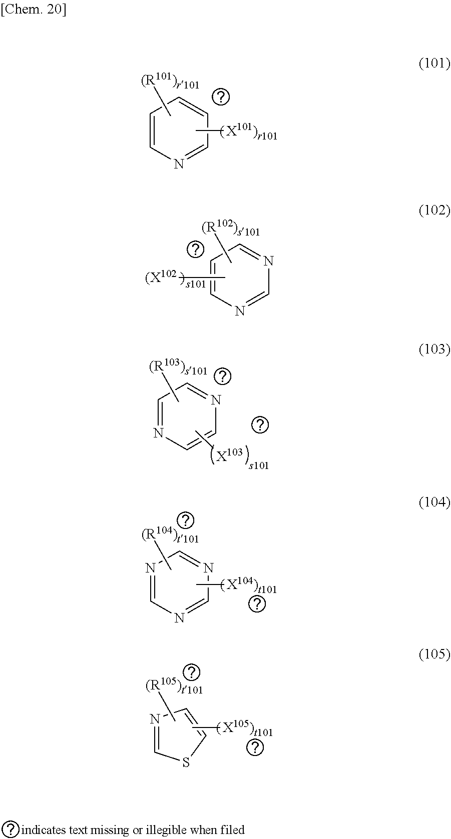

wherein
X¹⁰¹, X¹⁰², X¹⁰³, X¹⁰⁴ and X¹⁰⁵ are each independently represented by the formula (111):

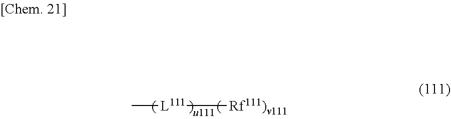

wherein
R¹⁰¹, R¹⁰², R¹⁰³, R¹⁰⁴ and R¹⁰⁵ are each independently represented by the formula (121):

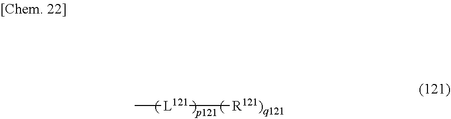

L¹¹¹ and L¹²¹ each independently denote
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR¹¹¹;
Rf¹¹¹ each independently denotes a structure represented by the formula (001);
R¹¹¹ and R¹²¹ each independently denote
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 3 to 18 carbon atoms, or
H, D (deuterium atom) or F (fluorine atom); and
r101 denotes an integer in the range of 1 to 5, and r′101 denotes an integer in the range of 0 to 4, provided that (r101+r′101) is an integer of 5 or less.

s101 each independently denotes an integer in the range of 1 to 4, and s′101 each independently denotes an integer in the range of 0 to 3, provided that (s101+s′101) is an integer of 4 or less.

t101 each independently denotes an integer in the range of 1 to 3, and t′101 denotes an integer in the range of 0 to 2, provided that (t101+t′101) is an integer of 3 or less.

u111 each independently denotes an integer in the range of 0 to 12.

v111 each independently denotes an integer in the range of 1 to 6.

p121 each independently denotes an integer in the range of 0 to 12.

q121 each independently denotes an integer in the range of 1 to 6.

u111 in at least one X¹⁰¹, at least one X¹⁰², at least one X¹⁰³, at least one X¹⁰⁴ and at least one X¹⁰⁵ each independently denotes 0.

The compounds represented by the formulae (101), (102), (103), (104), (105), (111) and (121) are described in detail below. [X¹⁰¹ to X¹⁰⁵ and R¹⁰¹ to R¹⁰⁵]

In the formulae (101), (102), (103), (104) and (105), X¹⁰¹, X¹⁰², X¹⁰³, X¹⁰⁴ and X¹⁰⁵ are not bound to a nitrogen atom or a sulfur atom forming a ring. Thus, X¹⁰¹, X¹⁰², X¹⁰³, X¹⁰⁴ and X¹⁰⁵ are bound to a carbon atom forming a ring. A carbon atom to which X¹⁰¹, X¹⁰², X¹⁰³, X¹⁰⁴ or X¹⁰⁵ is not bound can be bound to R¹⁰¹, R¹⁰², R¹⁰³, R¹⁰⁴ or R¹⁰⁵. X¹⁰¹, X¹⁰², X¹⁰³, X¹⁰⁴, X¹⁰⁵, R¹⁰¹, R¹⁰², R¹⁰³, R¹⁰⁴ or R¹⁰⁵ may be bound to any position. One carbon atom is bound to one, but not two or more, of X¹⁰¹, X¹⁰², X¹⁰³, X¹⁰⁴, X¹⁰⁵, R¹⁰¹, R¹⁰², R¹⁰³, R¹⁰⁴ and R¹⁰⁵.

r¹⁰¹ denotes an integer in the range of 1 to 5, and r′101 denotes an integer in the range of 0 to 4, provided that (r¹⁰¹+r′101) is an integer of 5 or less.

r¹⁰¹ preferably denotes an integer in the range of 1 to 4, more preferably an integer in the range of 1 to 3, still more preferably 1 or 2.

r′¹⁰¹ preferably denotes an integer in the range of 1 to 4, more preferably an integer in the range of 1 to 3, still more preferably 1 or 2.

s¹⁰¹ each independently denotes an integer in the range of 1 to 4, and s′101 each independently denotes an integer in the range of 0 to 3, provided that (s¹⁰¹+s′¹⁰¹) is an integer of 4 or less.

S¹⁰¹ preferably denotes an integer in the range of 1 to 3, more preferably 1 or 2.

s′¹⁰¹ preferably denotes an integer in the range of 1 to 3, more preferably 1 or 2.

t¹⁰¹ each independently denotes an integer in the range of 1 to 3, and t′101 denotes an integer in the range of 0 to 2, provided that (t¹⁰¹+t′101) is an integer of 3 or less.

In the formula (104), t¹⁰¹ is preferably 2 or 3, more preferably 2.

In the formula (104), t′101 is preferably 1 or 2, more preferably 1.

In the formula (105), t¹⁰¹ is preferably 1 or 2, more preferably 1.

In the formula (105), t′101 is preferably 1 or 2, more preferably 2.

### [Formula (111)]

X¹⁰¹ to X¹⁰⁵ are represented by the formula (111):

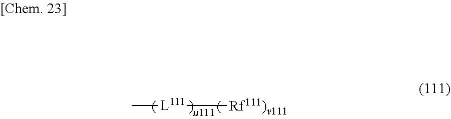

Rf¹¹¹ each independently denotes a structure represented by the formula (001);
L¹¹¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR¹¹¹.

u111 each independently denotes an integer in the range of 0 to 12.

v111 each independently denotes an integer in the range of 1 to 6.

The formula (111) is not particularly limited and may have a structure, for example, represented by one of the following (AA1) to (AA21).

The mark “*” in the following structures denotes a binding site.

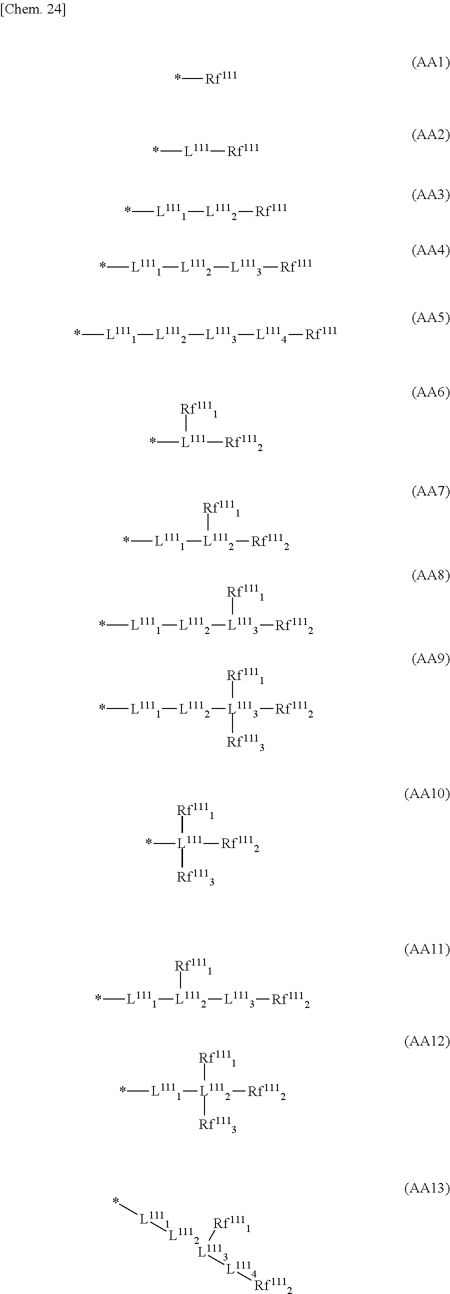

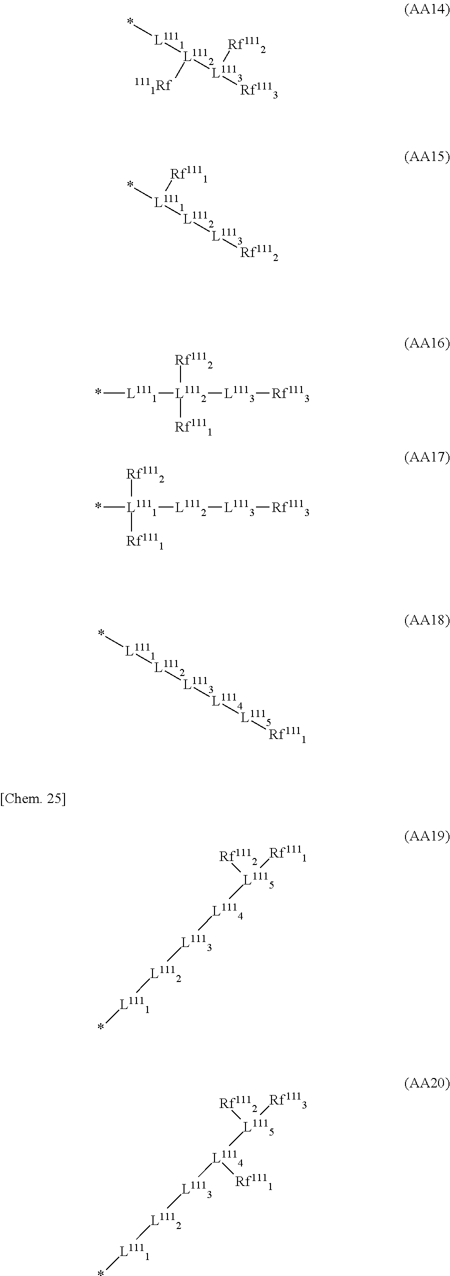

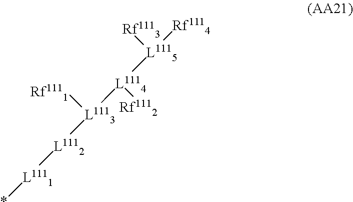

### [Rf¹¹¹]

Rf¹¹¹ denotes a structure represented by the formula (001);

v111 each independently denotes an integer in the range of 1 to 6. v111 preferably denotes an integer in the range of 1 to 5, more preferably an integer in the range of 1 to 4, still more preferably an integer in the range of 1 to 3, still more preferably 1 or 2. [L¹¹¹]

L¹¹¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR¹¹¹.

Preferably, the monocyclic, linked or condensed divalent to tetravalent aromatic hydrocarbon group represented by L¹¹¹ is a phenyl group or has a structure in which a plurality of benzene rings are linked or condensed.

Preferably, the monocyclic, linked or condensed divalent to tetravalent heteroaromatic group represented by L¹¹¹ has N, O or S as a heteroatom and is a 5-membered ring, a 6-membered ring or a structure in which these or a plurality of benzene rings are condensed.

More preferably, the cyclic divalent to tetravalent heteroaliphatic hydrocarbon group represented by L1 or L2 has N, O or S as a heteroatom and is a 5-membered ring, a 6-membered ring or a condensed structure thereof.

A compound that provides the monocyclic, linked or condensed divalent to tetravalent aromatic hydrocarbon group with 6 to 26 carbon atoms represented by L¹¹¹ is more preferably benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene.

A compound that provides the monocyclic, linked or condensed divalent to tetravalent heteroaromatic group is more preferably pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene.

A compound that provides the linear, branched or cyclic divalent to tetravalent aliphatic hydrocarbon group is preferably adamantane, diamantane, cyclohexane or methane.

A compound that provides the cyclic divalent to tetravalent heteroaliphatic hydrocarbon group is preferably morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

Still more preferably, the monocyclic, linked or condensed divalent to tetravalent aromatic hydrocarbon group represented by L¹¹¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene or triptycene,
the monocyclic, linked or condensed divalent to tetravalent heteroaromatic group is pyridine, pyrimidine, pyrazine, triazine, carbazole, furan, thiophene, benzofuran, benzothiophene, dibenzofuran, dibenzothiophene, thiazole, phenothiazine, dibenzo-1,4-dioxin or benzothiazole,
the linear, branched or cyclic divalent to tetravalent aliphatic hydrocarbon group is adamantane, diamantane, cyclohexane or methane, and
   a compound that provides the cyclic divalent to tetravalent heteroaliphatic hydrocarbon group is morpholine, piperazine, homopiperazine, hexahydro-1,3,5-triazine, 1,4-dioxin, 1,4-dithiane or 1,4,7,10-tetraazacyclododecane.

Still more preferably, the monocyclic, linked or condensed divalent to tetravalent aromatic hydrocarbon group represented by L¹¹¹ is phenyl, biphenyl, naphthalene, fluorene, spirobifluorene, phenanthrene or triptycene,
the monocyclic, linked or condensed divalent to tetravalent heteroaromatic group is pyridine, pyrimidine, pyrazine, triazine, carbazole, furan, thiophene, benzofuran, benzothiophene, dibenzofuran, dibenzothiophene, thiazole, phenothiazine, dibenzo-1,4-dioxin or benzothiazole,
the linear, branched or cyclic divalent to tetravalent aliphatic hydrocarbon group is adamantane, diamantane or cyclohexane, and
a compound that provides the cyclic divalent to tetravalent heteroaliphatic hydrocarbon group is morpholine, piperazine, homopiperazine, hexahydro-1,3,5-triazine, 1,4-dioxin, 1,4-dithiane or 1,4,7,10-tetraazacyclododecane.

L¹¹¹ preferably each independently denotes
a monocyclic, linked or condensed divalent to tetravalent aromatic hydrocarbon group,
a monocyclic, linked or condensed divalent to tetravalent heteroaromatic group,
a linear, branched or cyclic divalent to tetravalent aliphatic hydrocarbon group,
a cyclic divalent to tetravalent heteroaliphatic hydrocarbon group, or
O or S,
L¹¹¹ more preferably each independently denotes
a monocyclic, linked or condensed divalent to tetravalent heteroaromatic group,
a linear, branched or cyclic divalent to tetravalent aliphatic hydrocarbon group,
a cyclic divalent to tetravalent heteroaliphatic hydrocarbon group, or
O or S.

When L¹¹¹ denotes a substituted aromatic hydrocarbon group, a substituted heteroaromatic group, a substituted aliphatic hydrocarbon group or a substituted cyclic heteroaliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a cyano group, a fluorine atom, a deuterium atom and a structure represented by the formula (001), and
a combination of these groups.

A substituent of L¹¹¹ is preferably each independently substituted with one or more groups selected from the group consisting of
a methyl group, a trifluoromethyl group, a trifluoromethoxy group, an alkyl group with 2 to 10 carbon atoms, a cyano group, a deuterium atom, a fluorine atom, a phenyl group, a biphenylyl group, a naphthyl group, a phenanthryl group, a pyridyl group, a pyrimidyl group, a pyrazyl group, a triazyl group, an indolyl group, a carbazolyl group, a benzothienyl group, a dibenzothienyl group, a benzofuranyl group, a dibenzofuranyl group, a thiazolyl group, a benzothiazolyl group and a structure represented by the formula (001), and
a combination of these groups.

A substituent of L¹¹¹ is more preferably each independently substituted with one or more groups selected from the group consisting of
a methyl group, a trifluoromethyl group, a trifluoromethoxy group, an alkyl group with 2 to 10 carbon atoms, a fluorine atom, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group, a thiazolyl group and a structure represented by the formula (001), and
a combination of these groups.

A substituent of L¹¹¹ is preferably each independently
(i) a methyl group, a methoxy group, an alkyl group with 2 to 10 carbon atoms, an alkoxy group with 2 to 10 carbon atoms, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group or a thiazolyl group,
(ii) the group represented by (i) substituted with one or more groups selected from the group consisting of a fluorine atom, a perfluoroalkyl group with 1 to 10 carbon atoms and a perfluoroalkoxy group with 1 to 10 carbon atoms, or
(iii) a fluorine atom or a structure represented by the formula (001).

A substituent of L¹¹¹ is more preferably each independently
(i′) a methyl group, an ethyl group, a butyl group, a methoxy group, an ethoxy group, butoxy group, a phenyl group, a naphthyl group, a pyridyl group, a pyrimidyl group, a pyrazyl group, a triazyl group, a carbazolyl group, a dibenzothienyl group, a dibenzofuranyl group or a thiazolyl group,
(ii′) the group represented by (i′) substituted with one or more groups selected from the group consisting of a fluorine atom, a perfluoroalkyl group with 1 to 6 carbon atoms and a perfluoroalkoxy group with 1 to 6 carbon atoms, or
(iii′) a fluorine atom or a structure represented by the formula (001).

A substituent of L¹¹¹ is still more preferably each independently
a trifluoromethyl group, a trifluoromethoxy group, an alkyl group having 2 to 10 carbon atoms and substituted with 3 or more fluorine atoms, an alkoxy group having 2 to 10 carbon atoms and substituted with 3 or more fluorine atoms, a structure represented by the formula (001) or a fluorine atom.

u111 each independently denotes an integer in the range of 0 to 12. u111 preferably denotes an integer in the range of 0 to 10, more preferably an integer in the range of 0 to 8, still more preferably an integer in the range of 0 to 6, still more preferably an integer in the range of 0 to 4.

u111 in at least one X¹⁰¹, at least one X¹⁰², at least one X¹⁰³, at least one X¹⁰⁴ and at least one X¹⁰⁵ each independently denotes 0.

### Specific Example of L¹¹¹

L¹¹¹ may have a structure, for example, represented by one of the following (AAB1) to (AAB803). The mark “*” in the following structures denotes a binding site.

F denotes a fluorine atom, v denotes an integer in the range of 0 to 5, w denotes an integer in the range of 0 to 4, x denotes an integer in the range of 0 to 3, y denotes an integer in the range of 0 to 2, and z denotes an integer of 0 or 1.

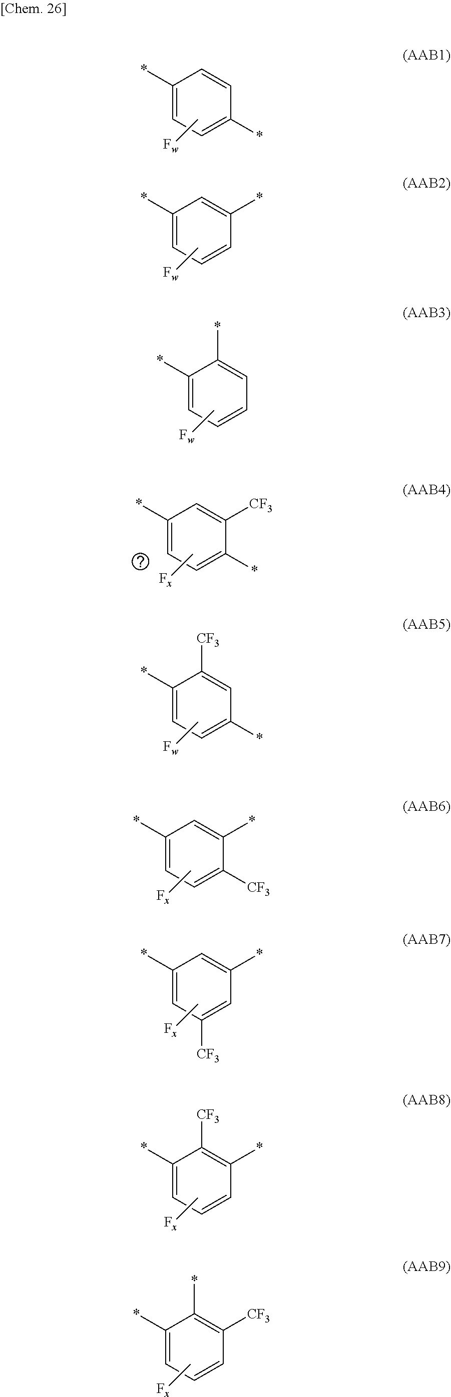

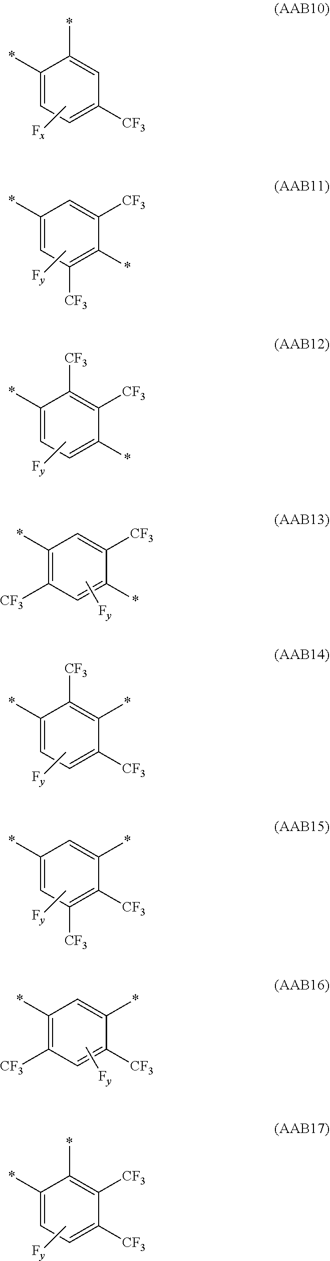

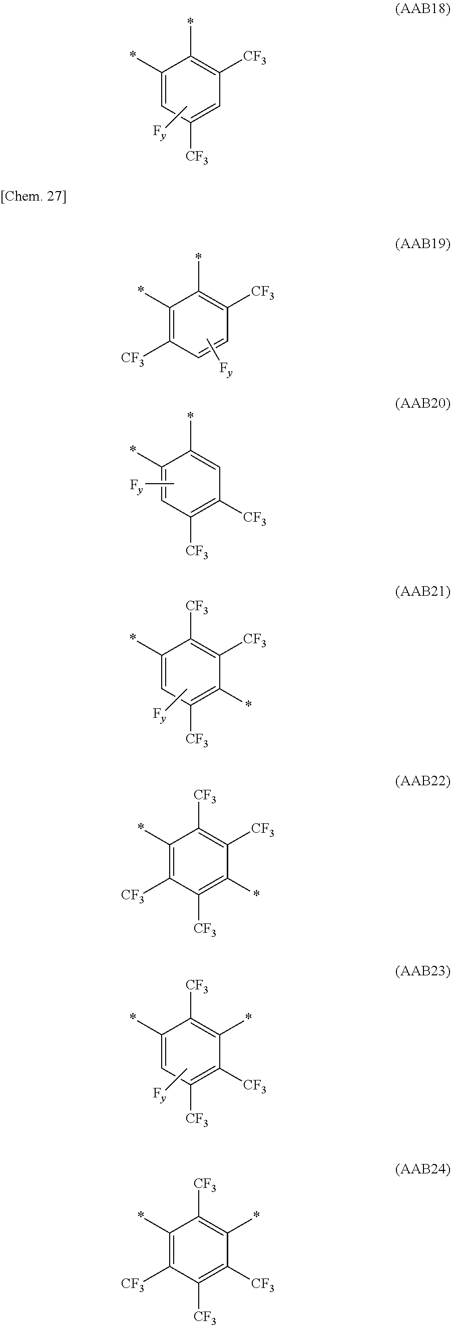

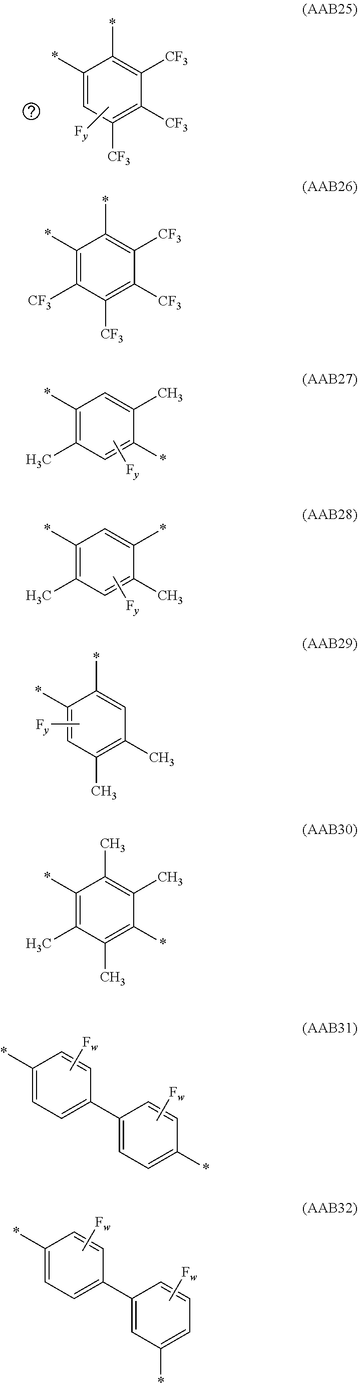

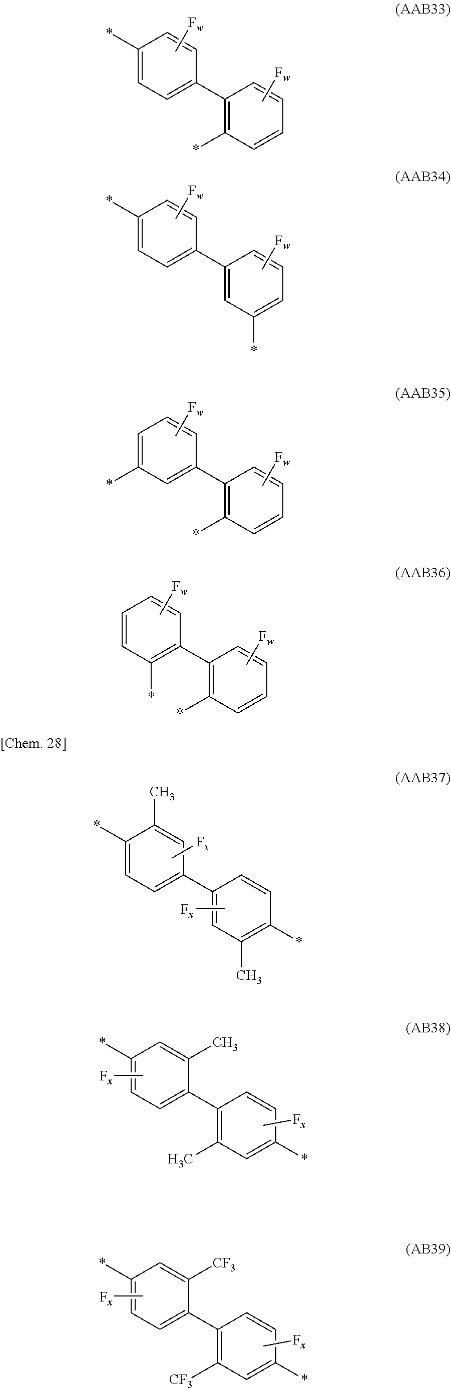

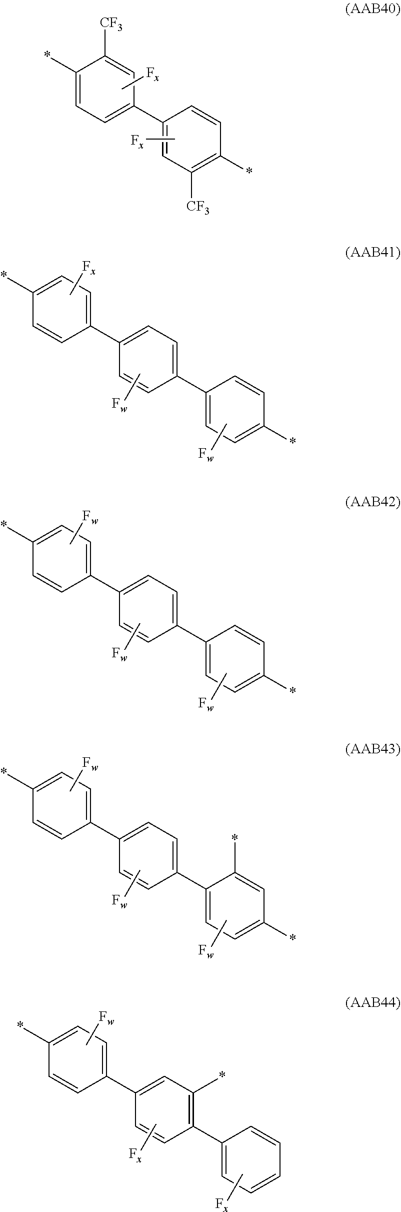

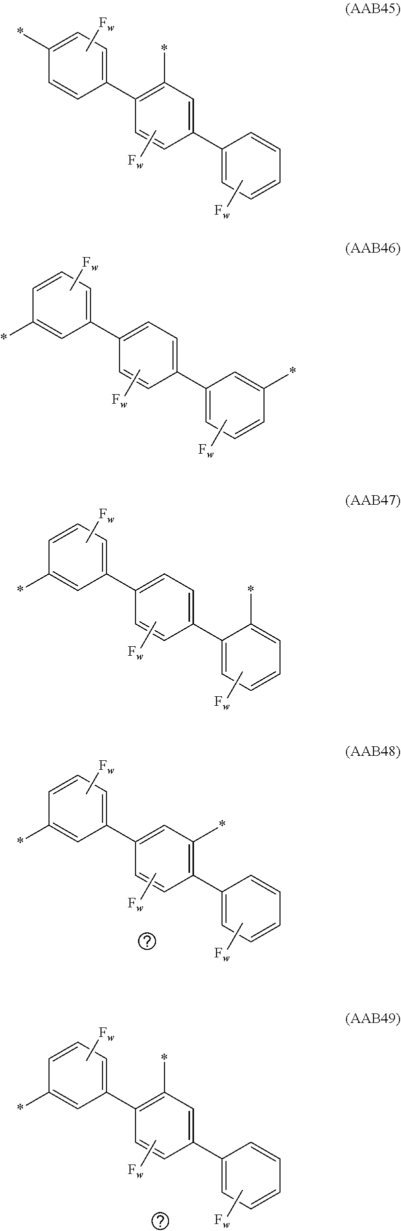

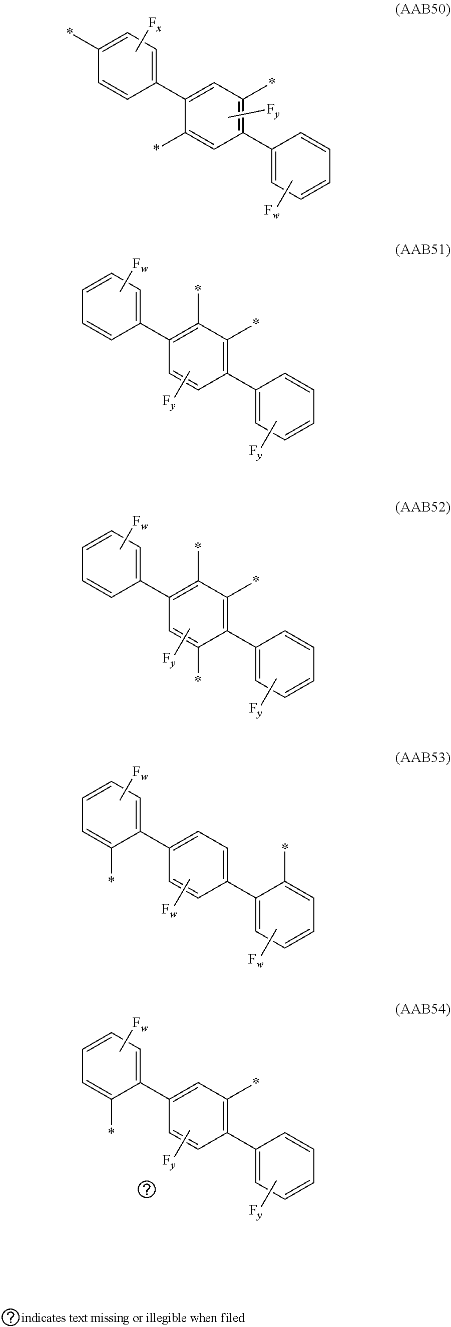

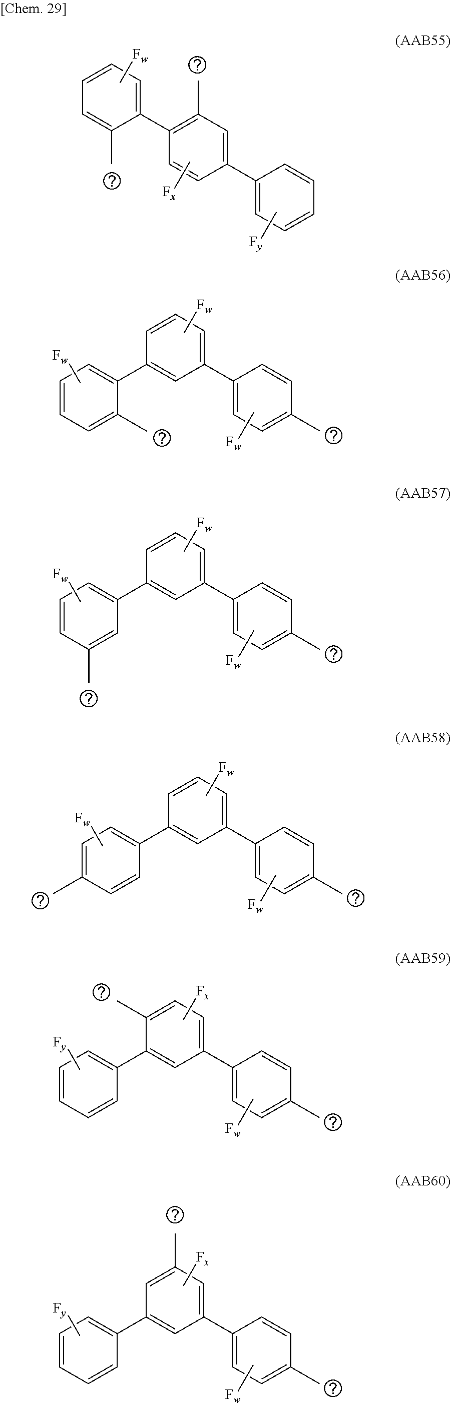

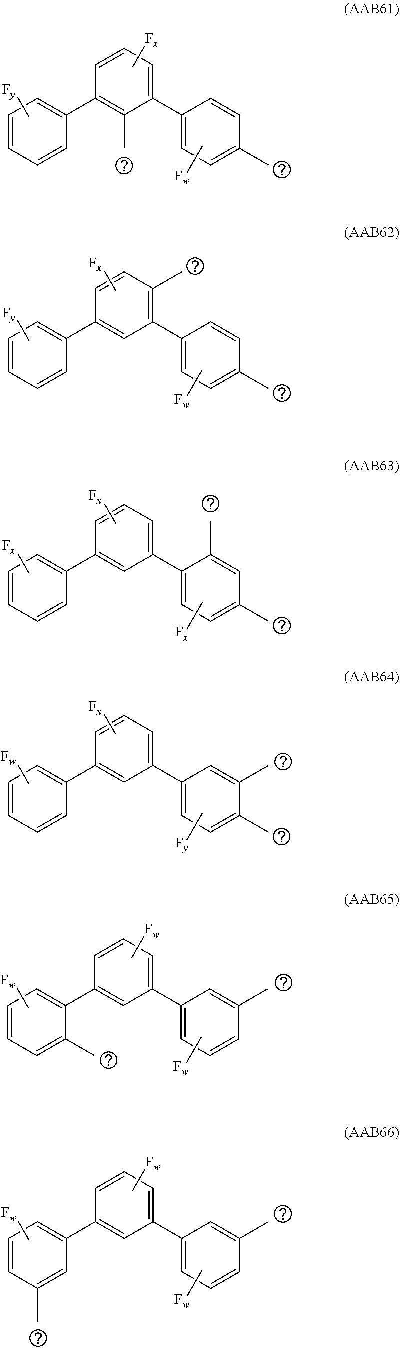

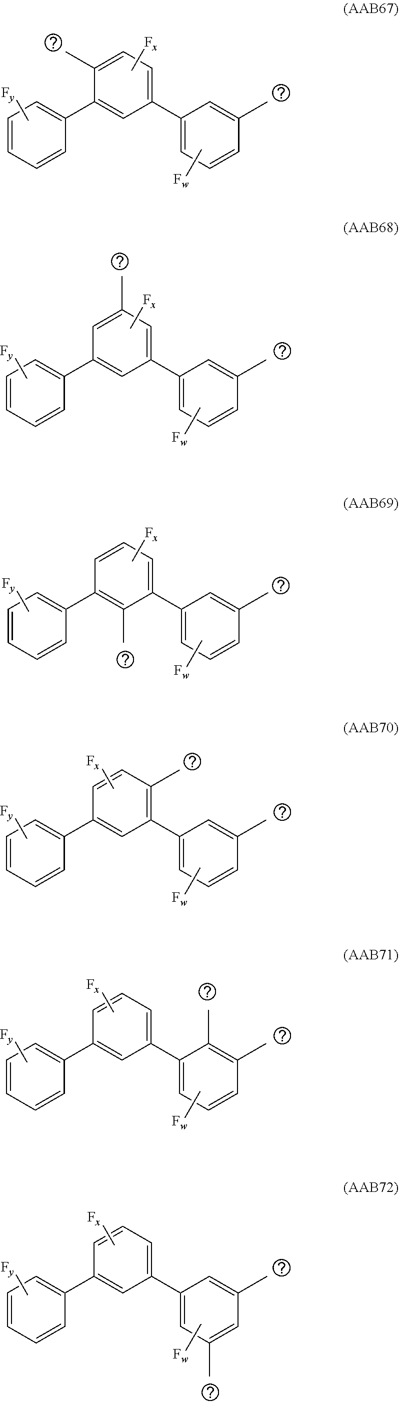

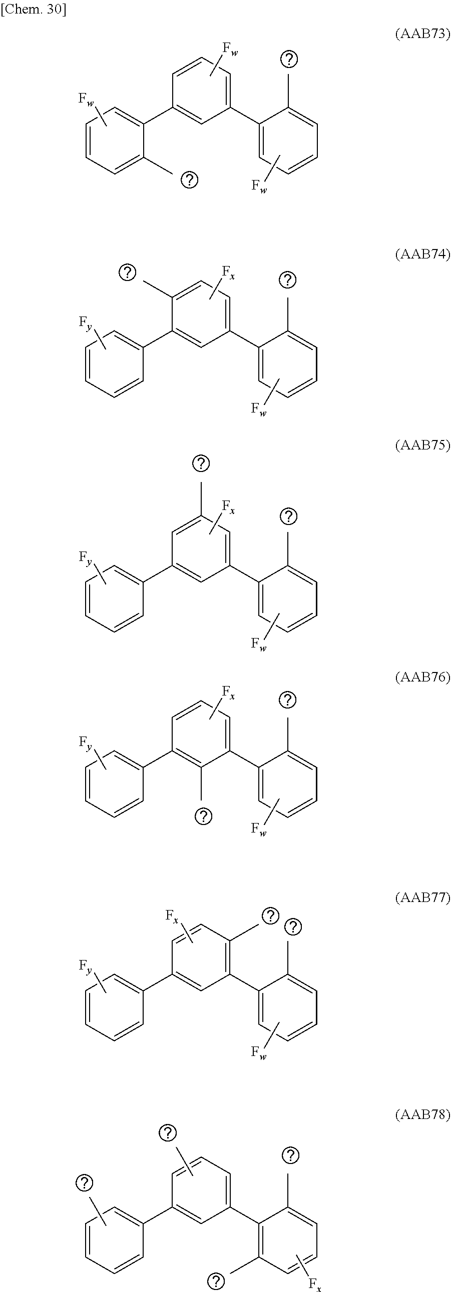

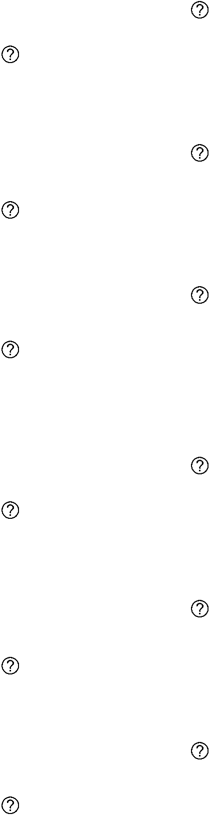

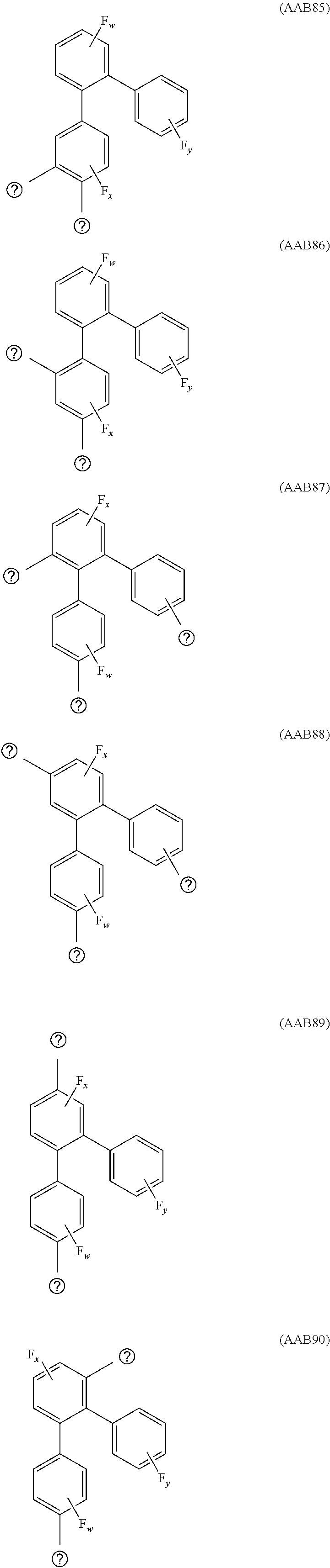

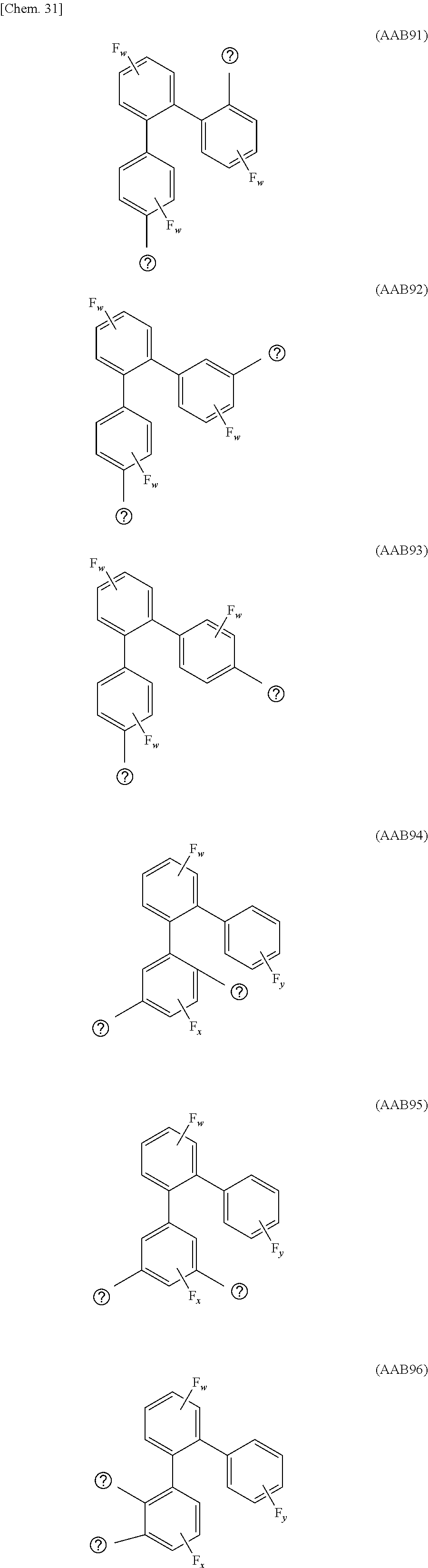

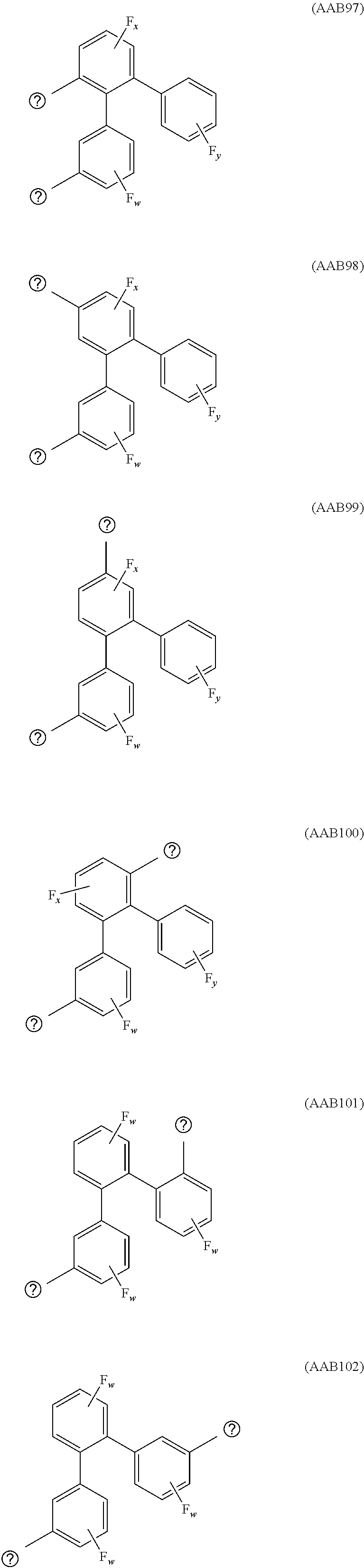

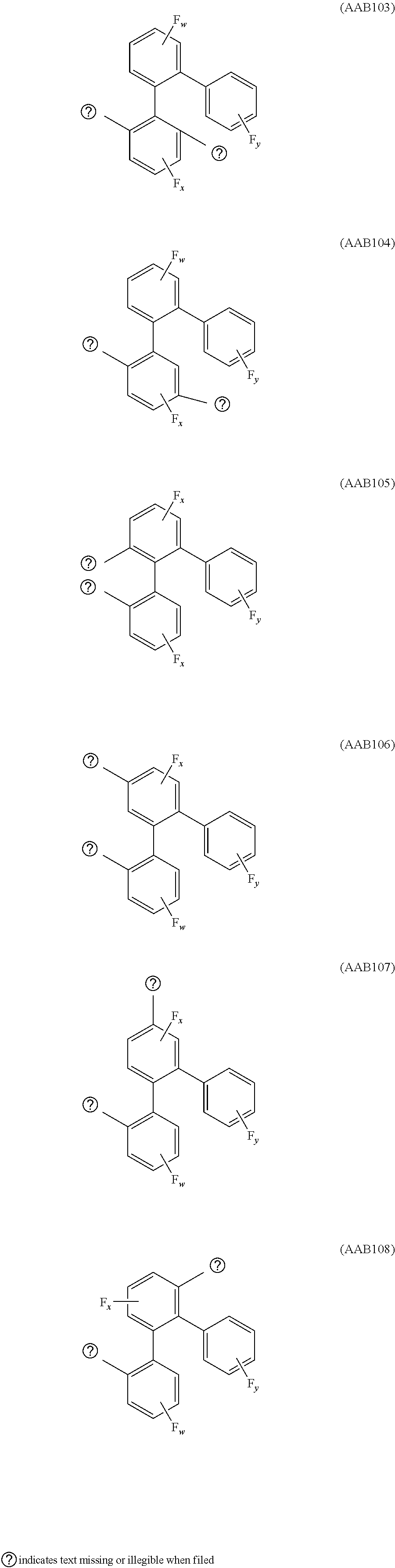

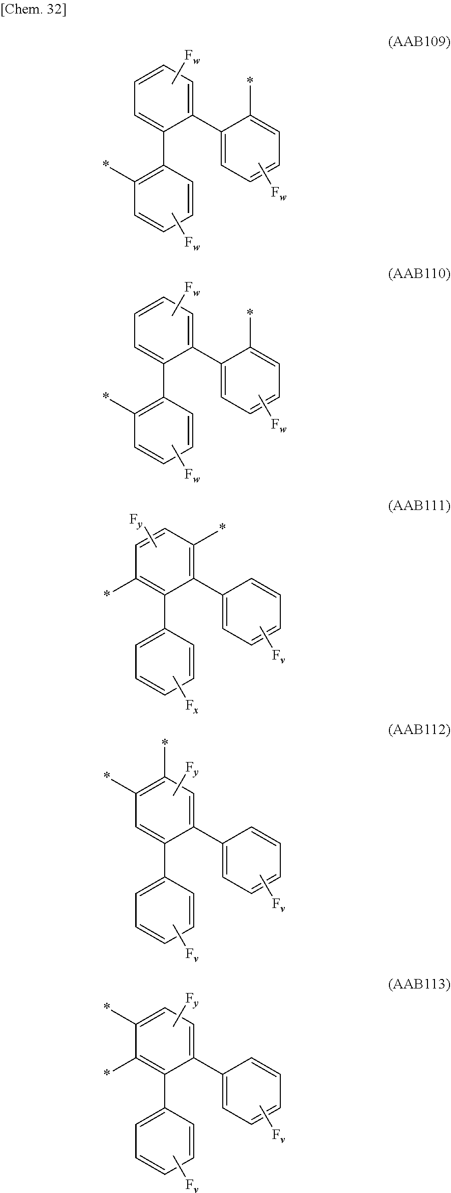

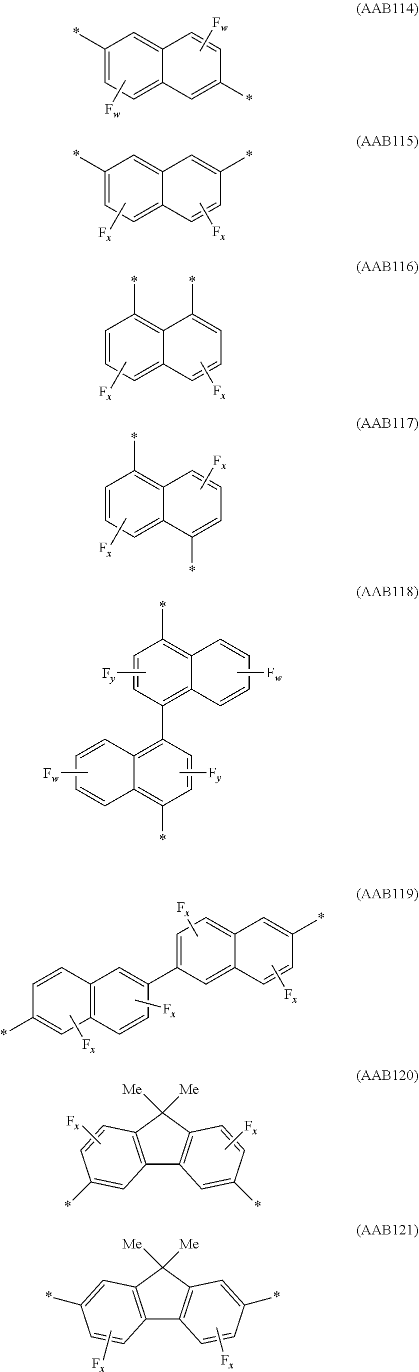

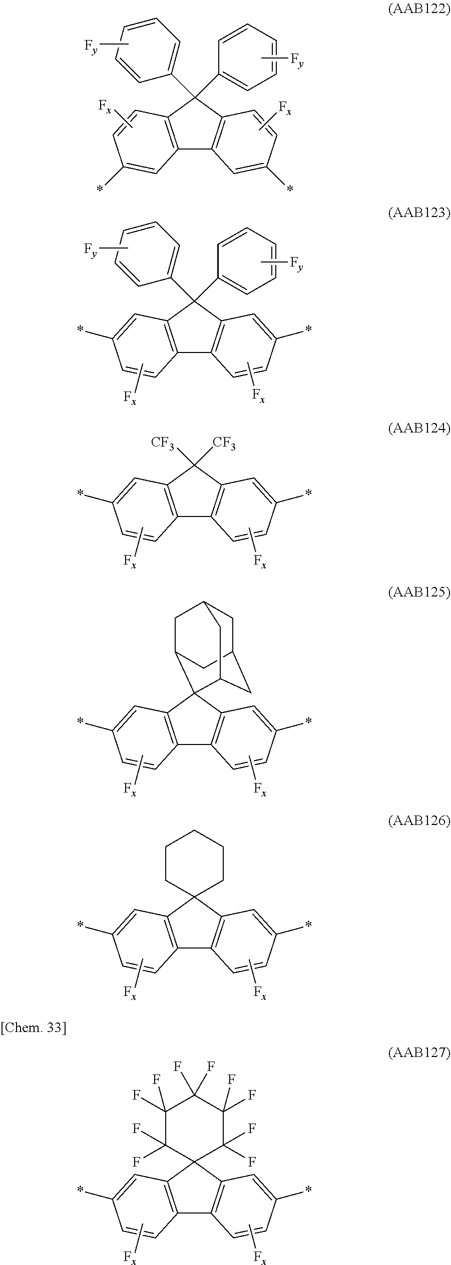

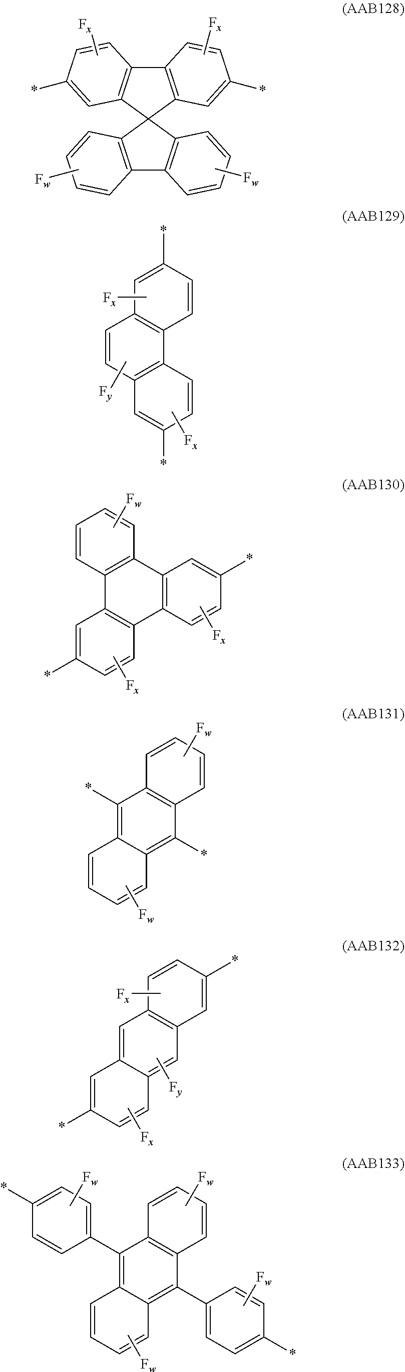

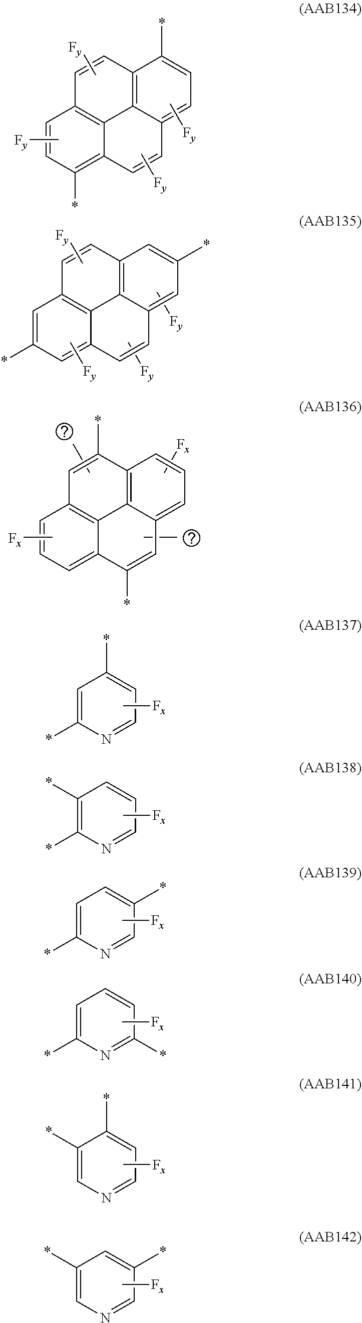

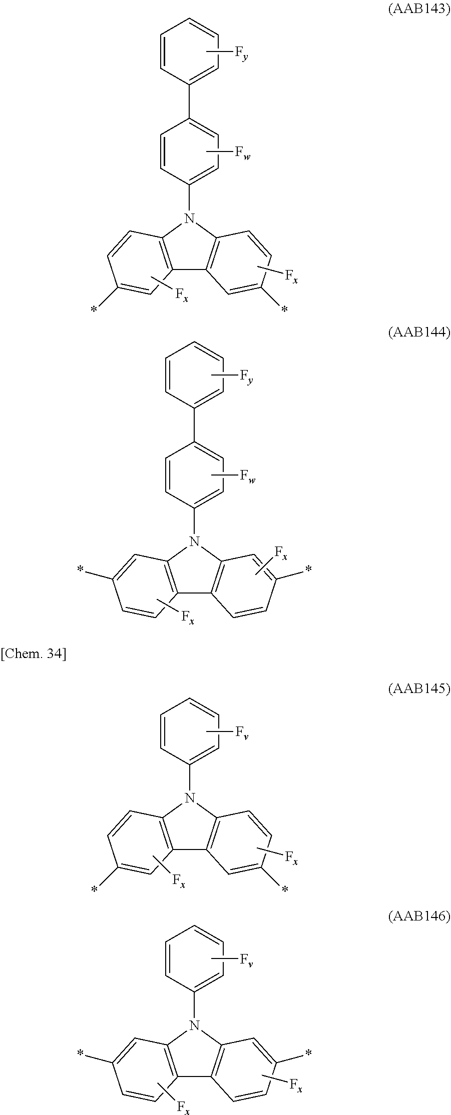

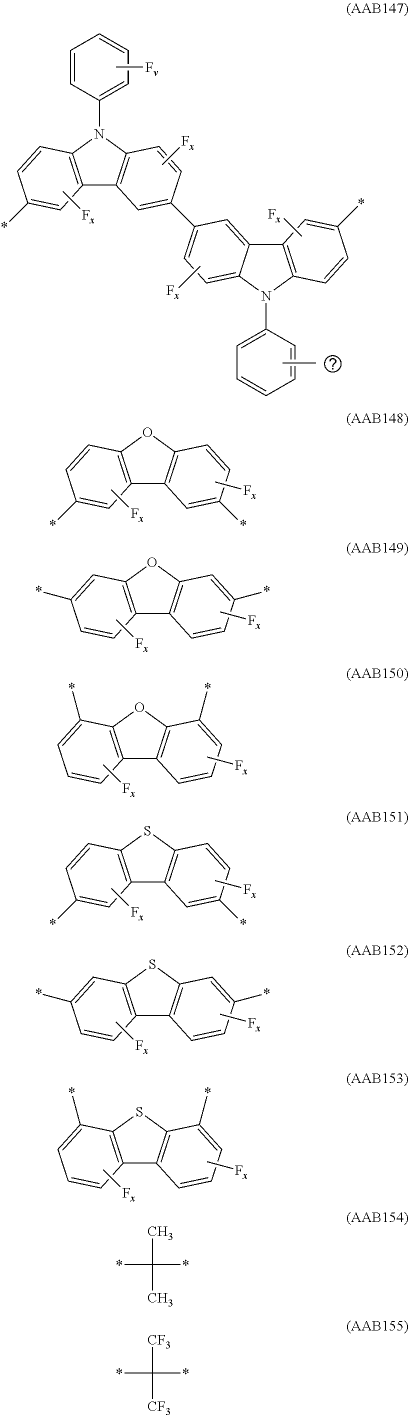

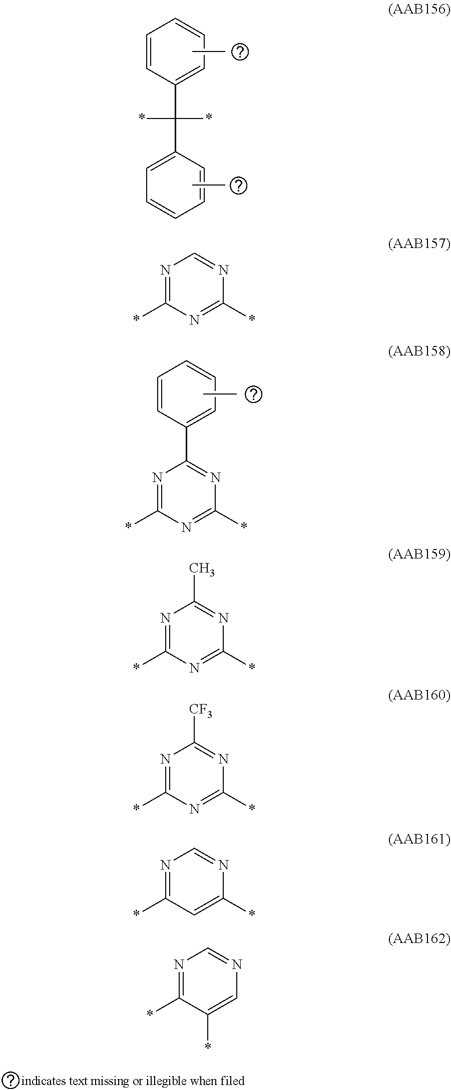

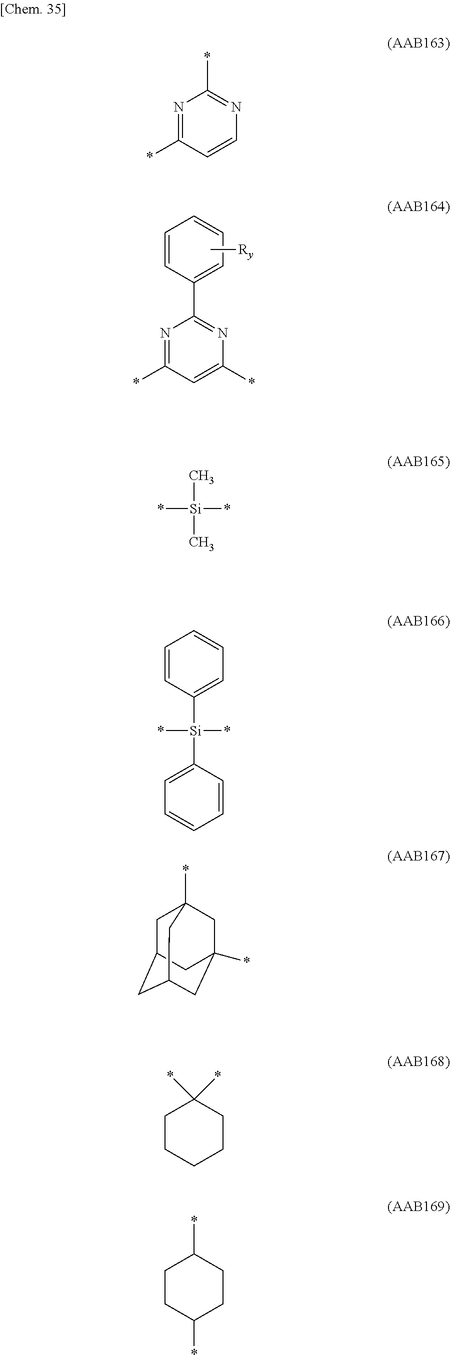

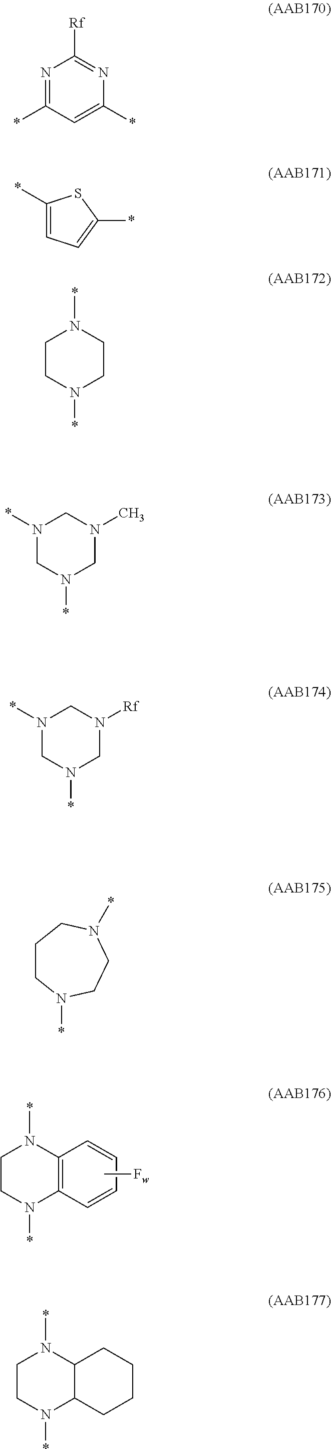

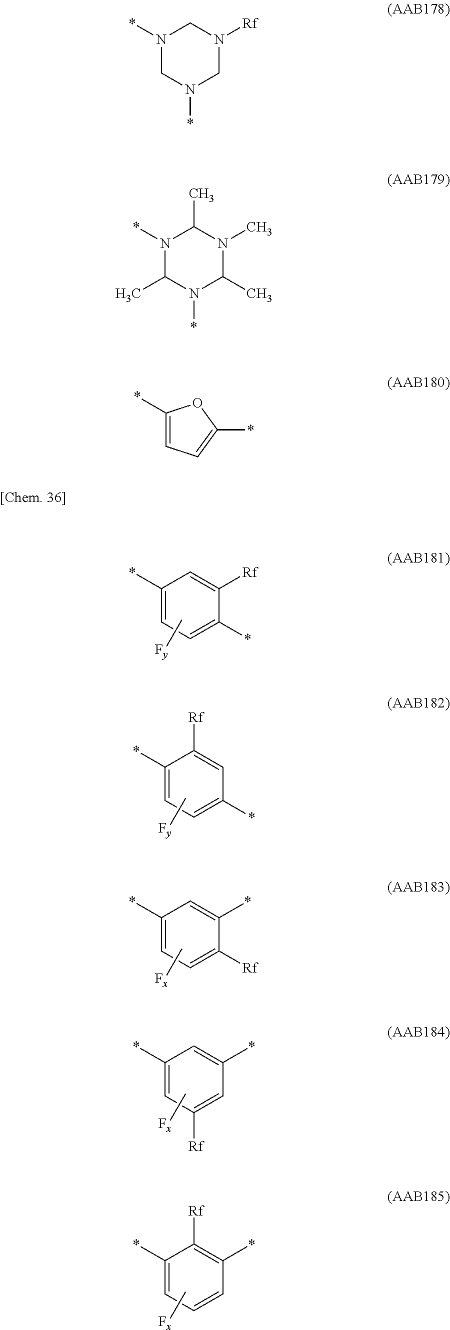

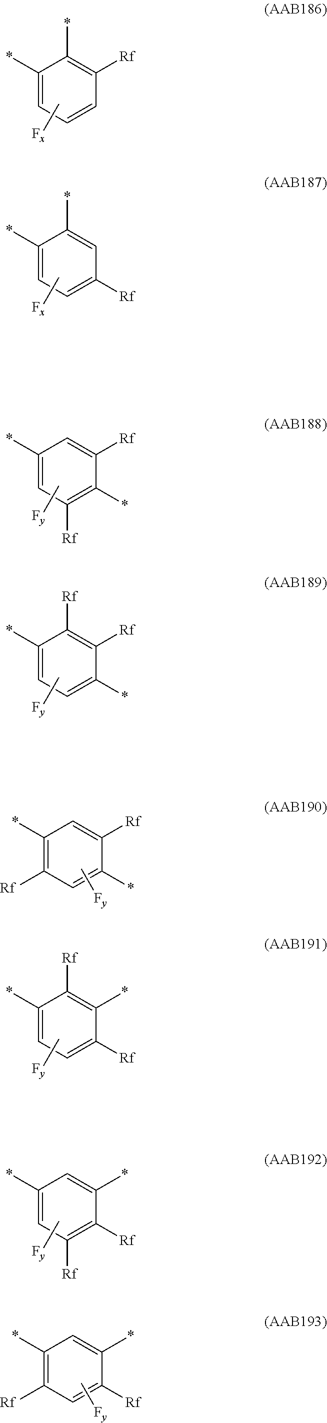

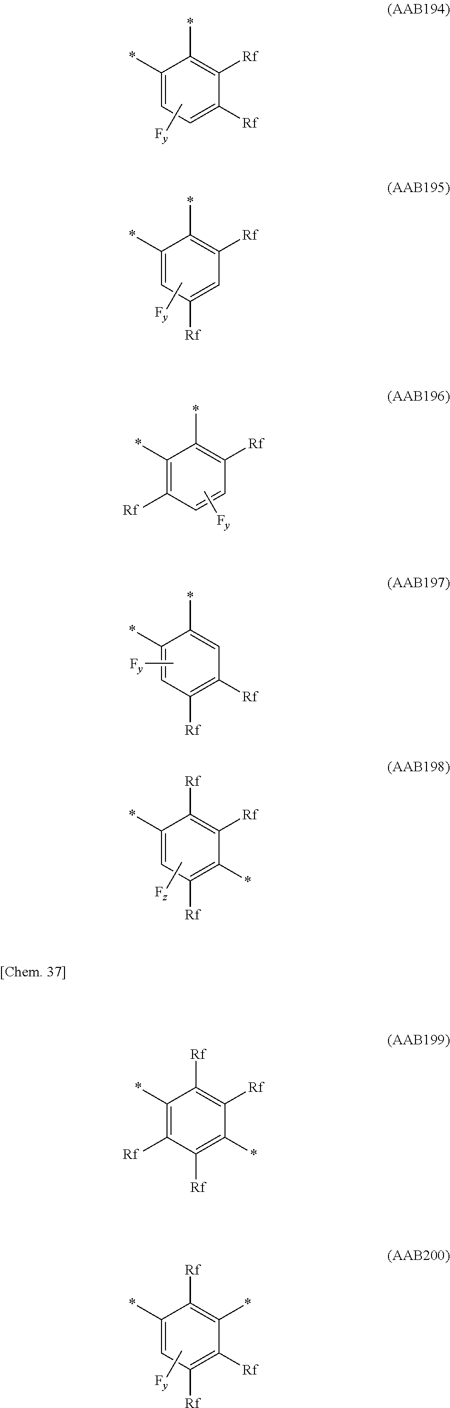

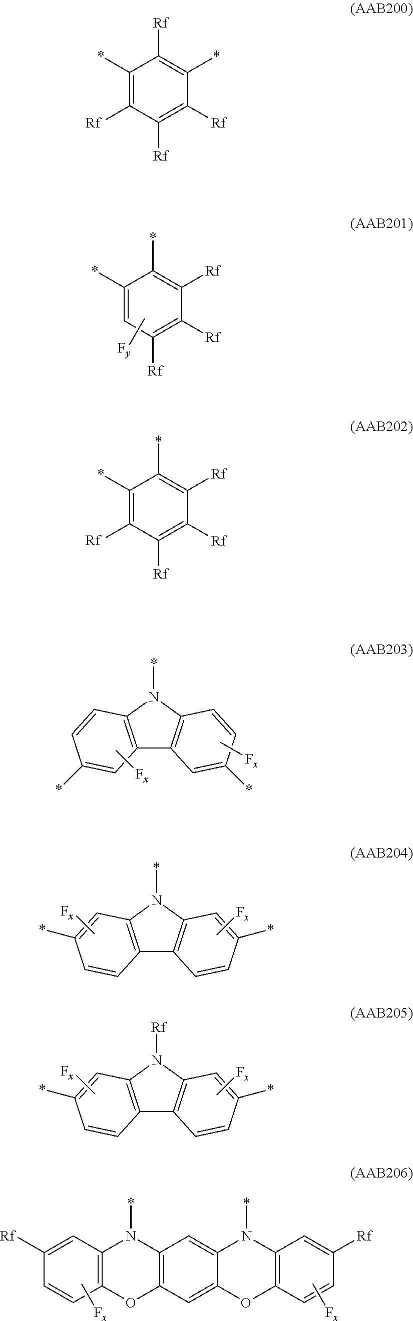

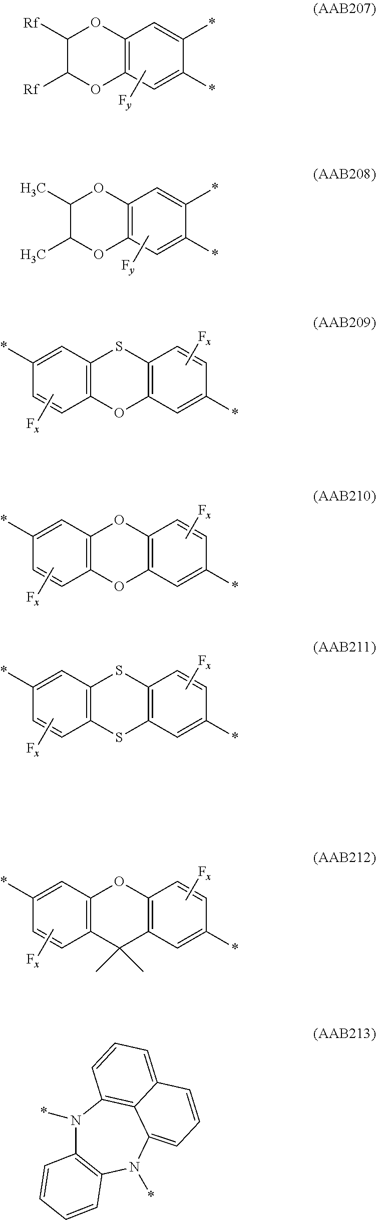

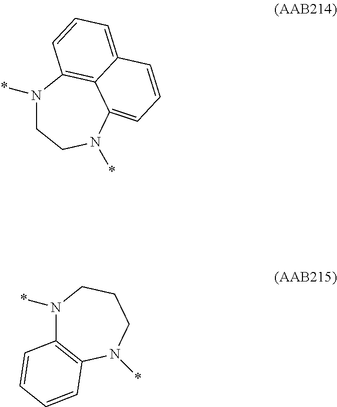 [SMILES: *N1CCN(*)c2cccc3cccc1c23 ; *N1CCCN(*)c2ccccc21]

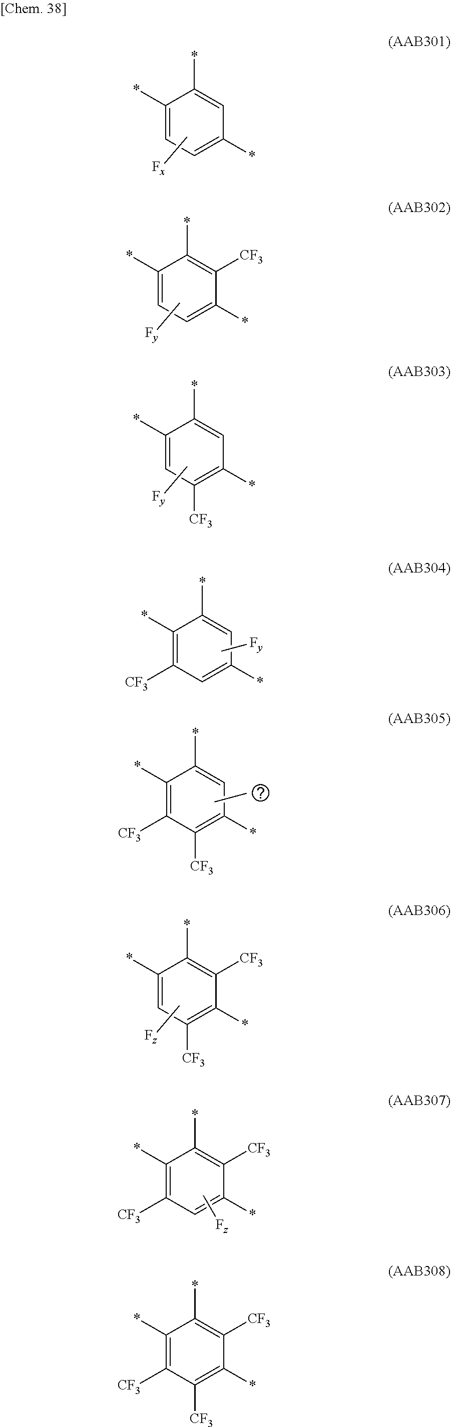

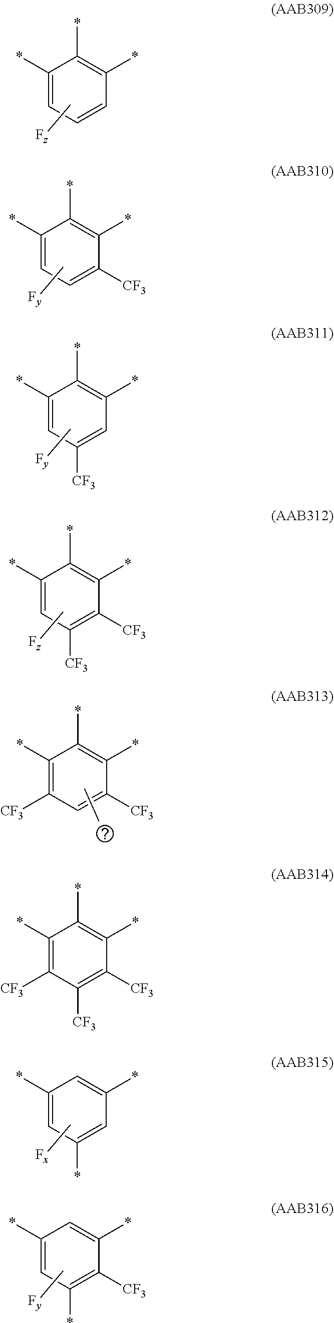

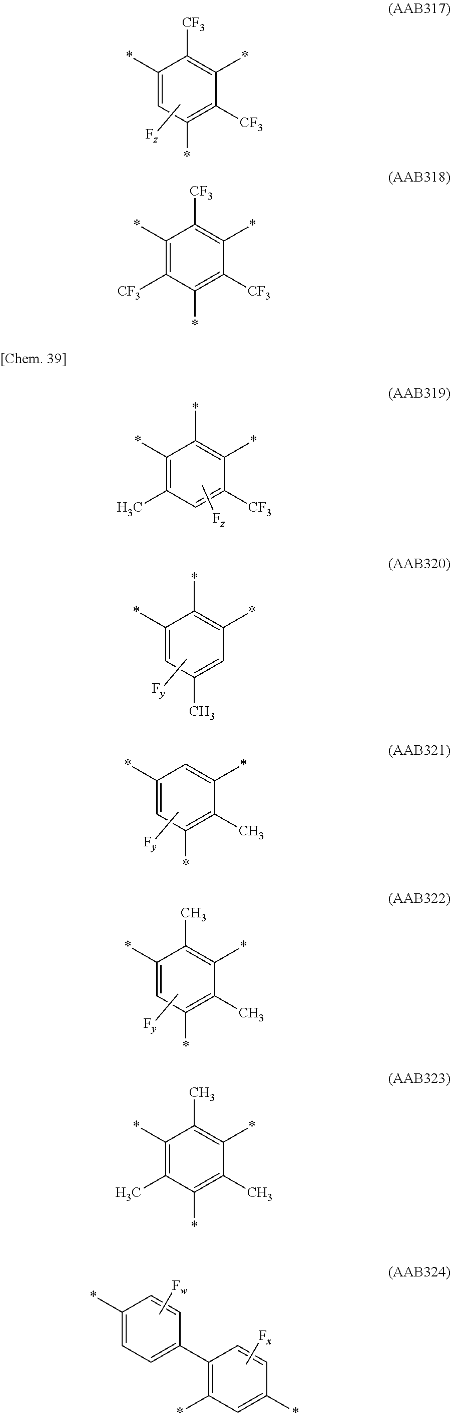

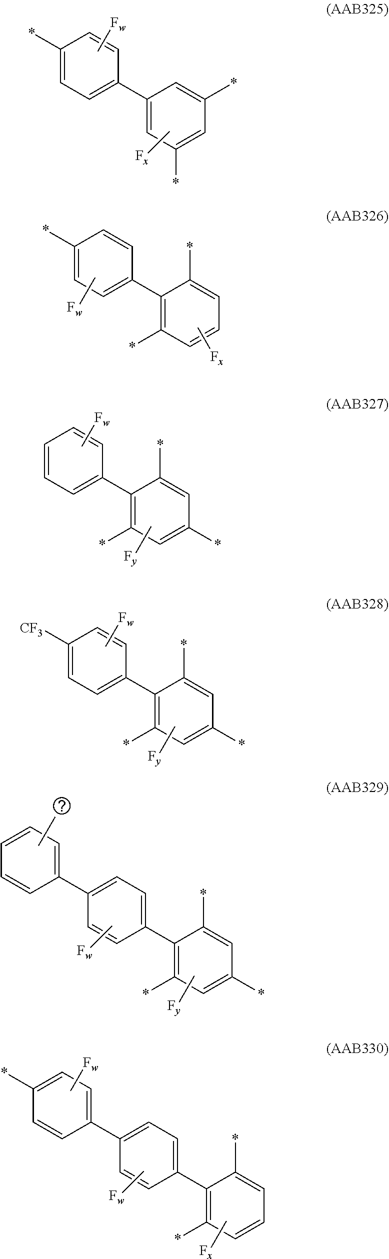

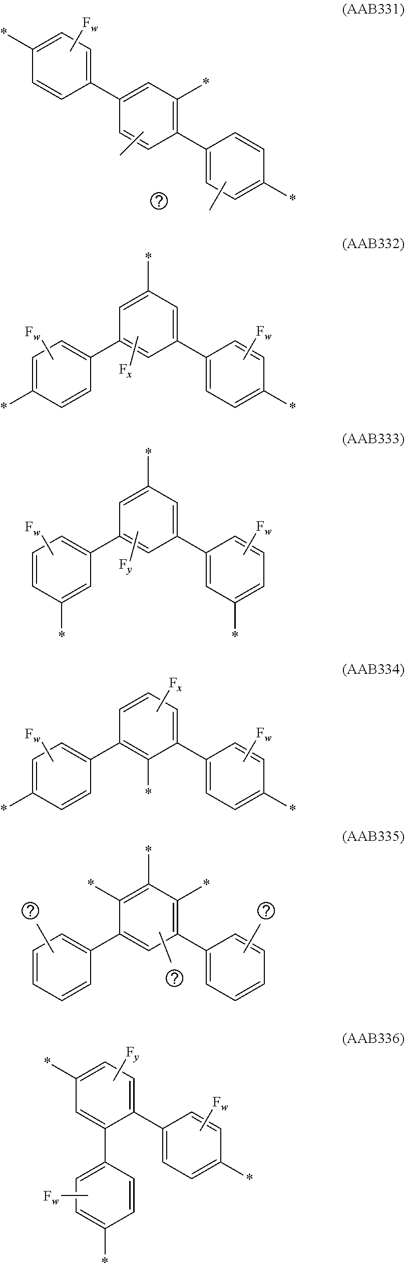

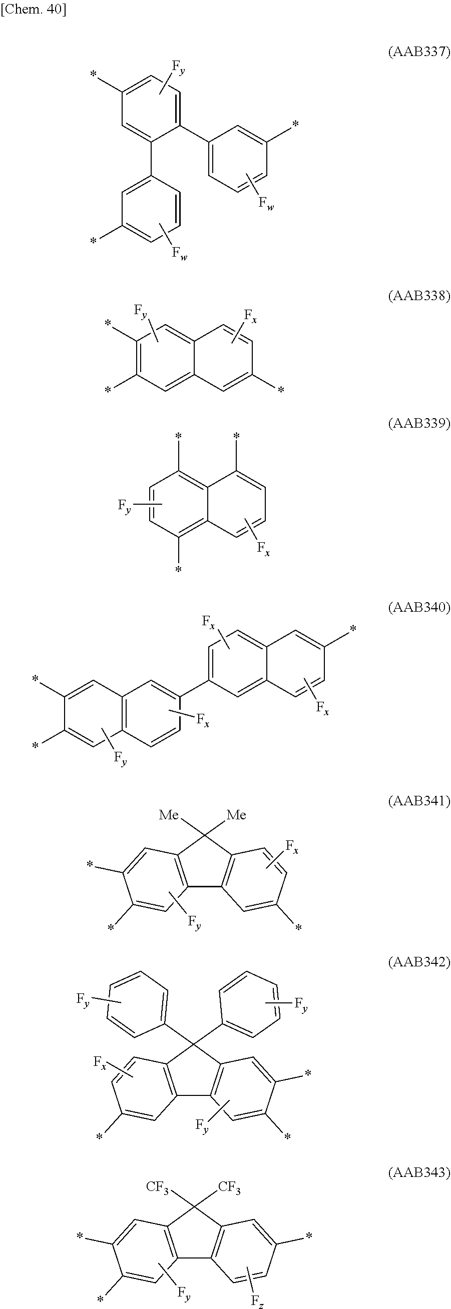

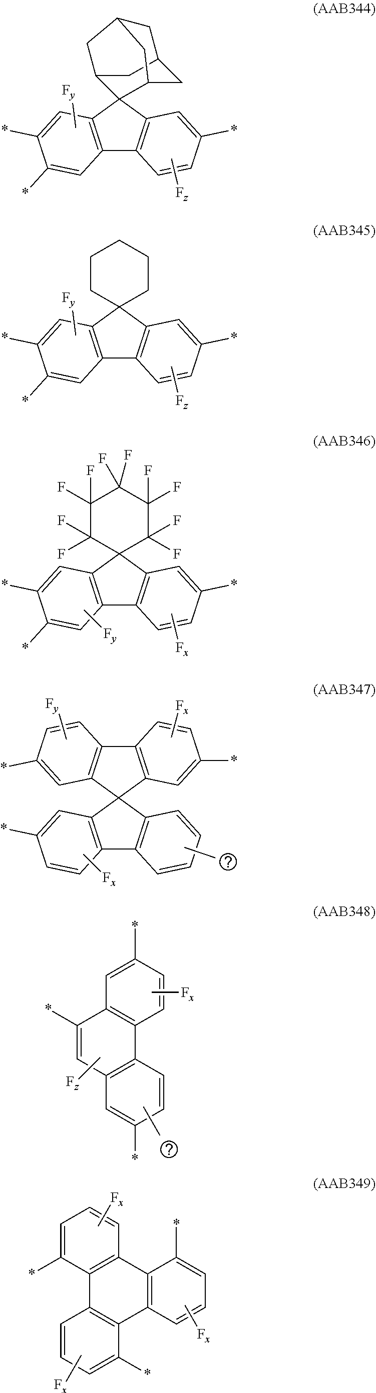

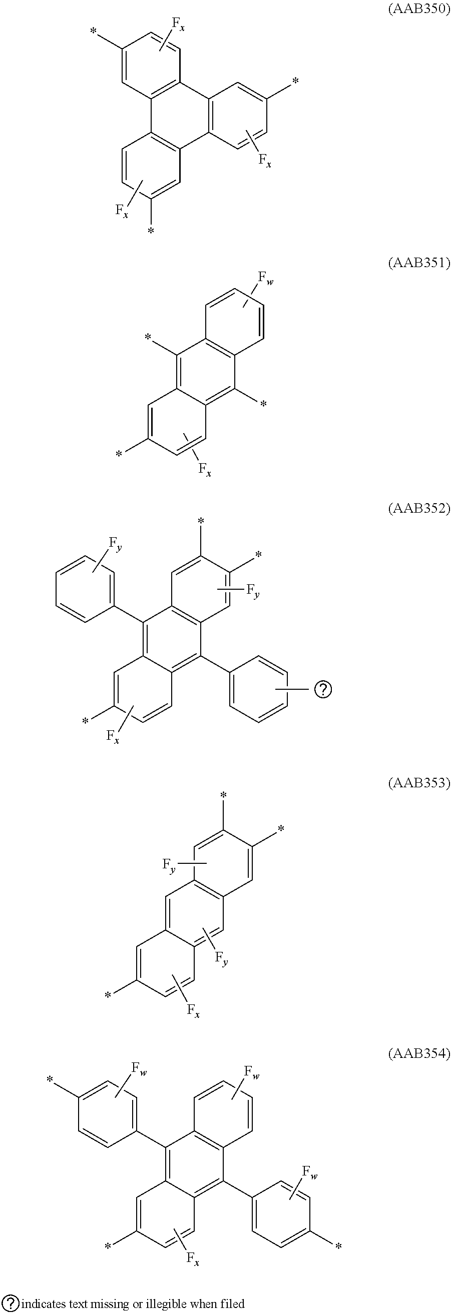

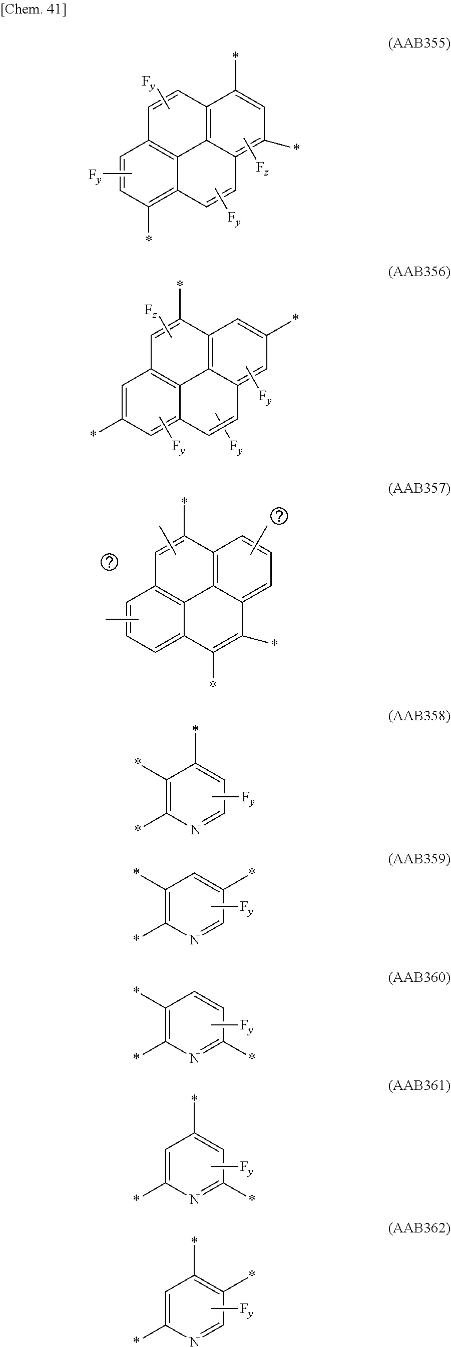

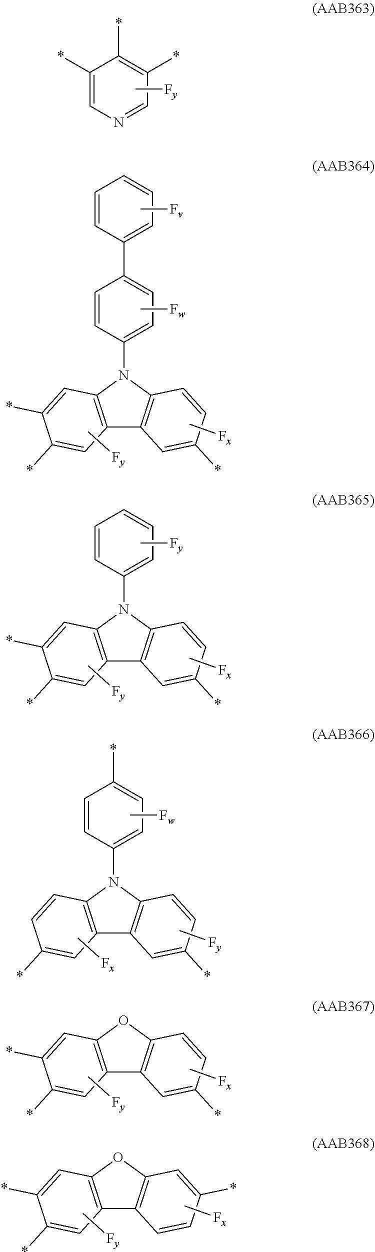

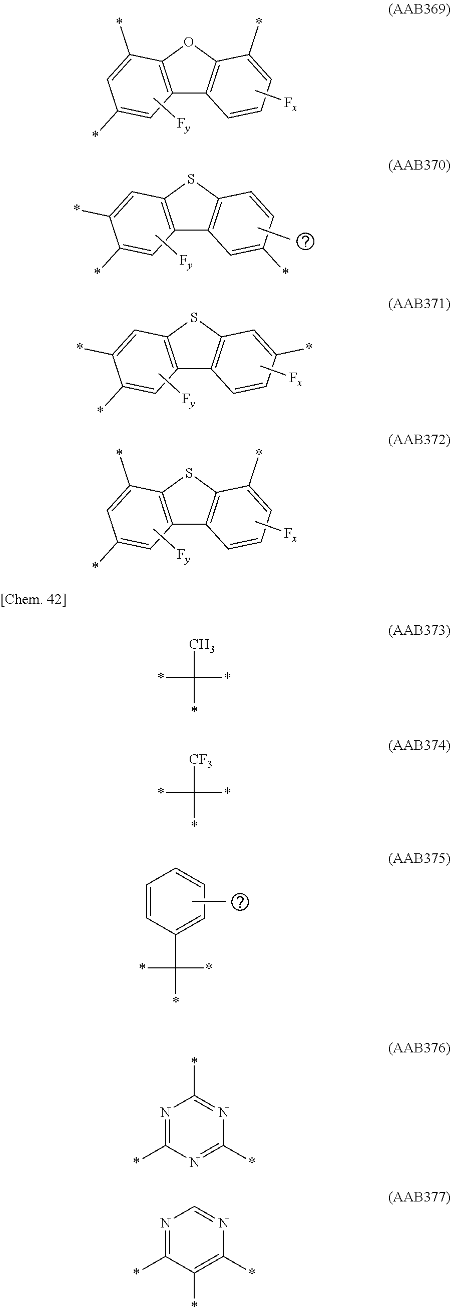

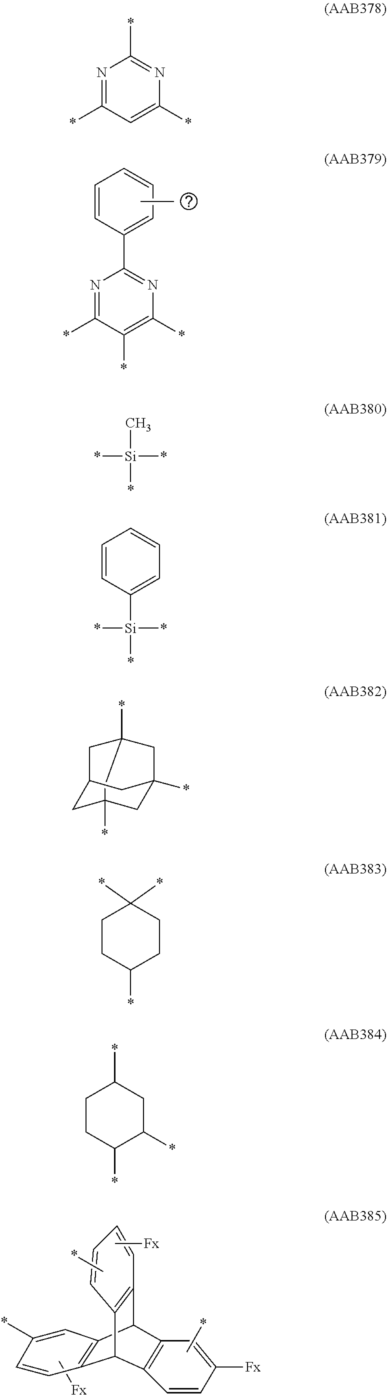

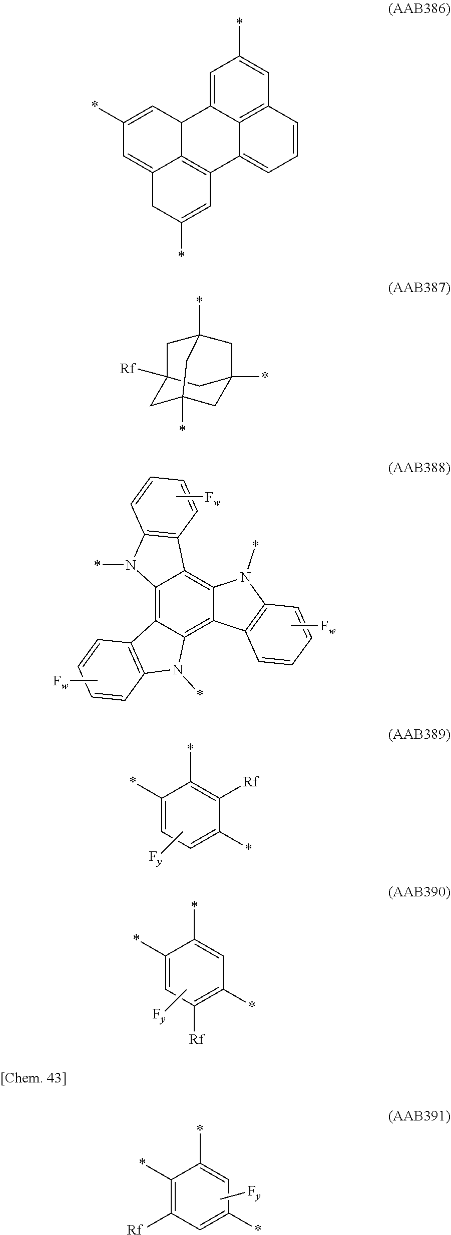

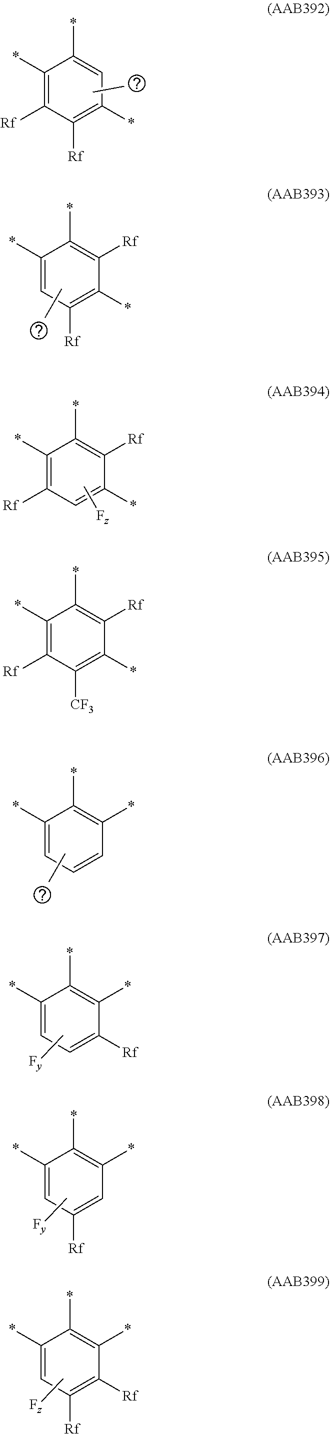

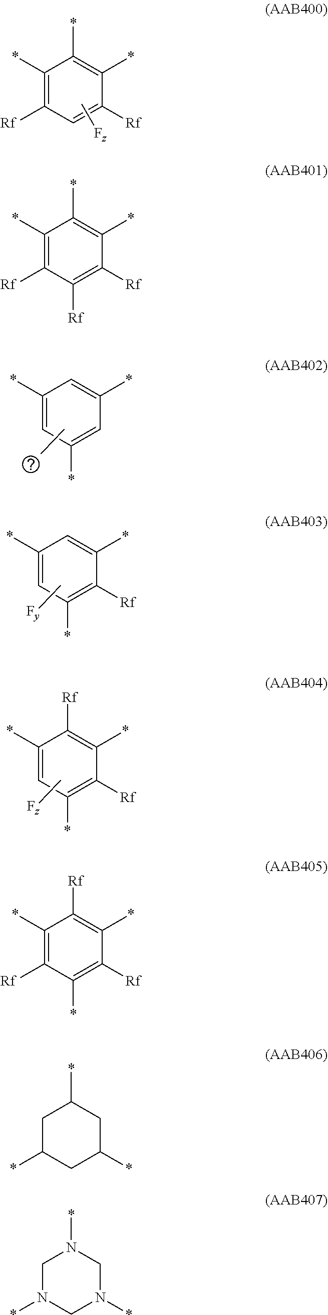

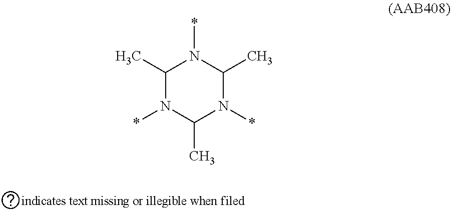 [SMILES: *N1C(C)N(*)C(C)N(*)C1C]

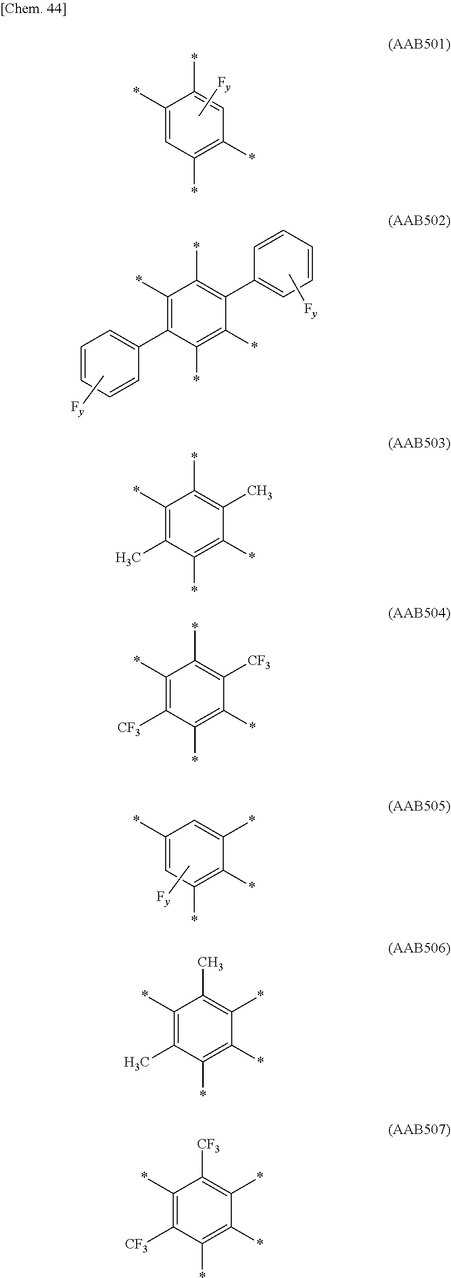

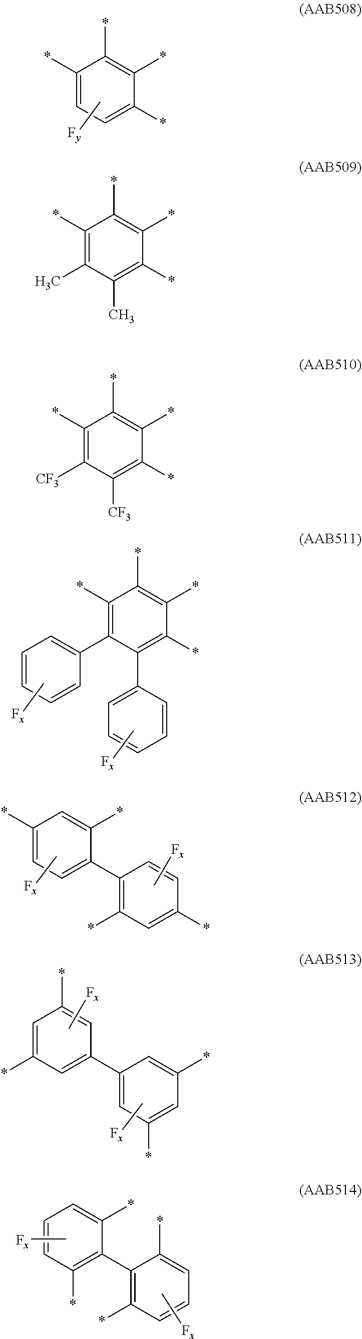

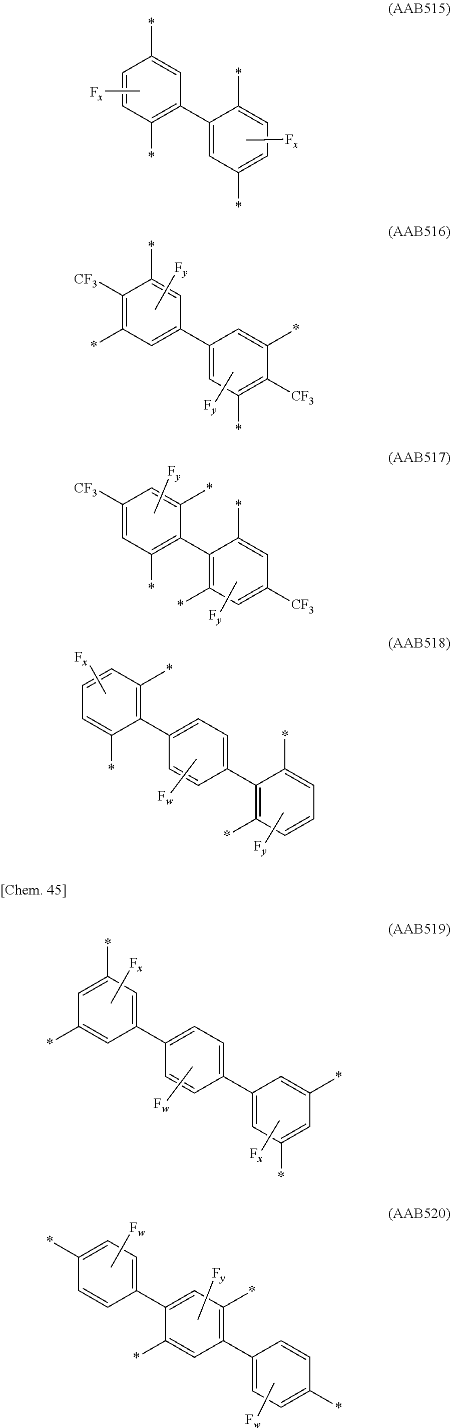

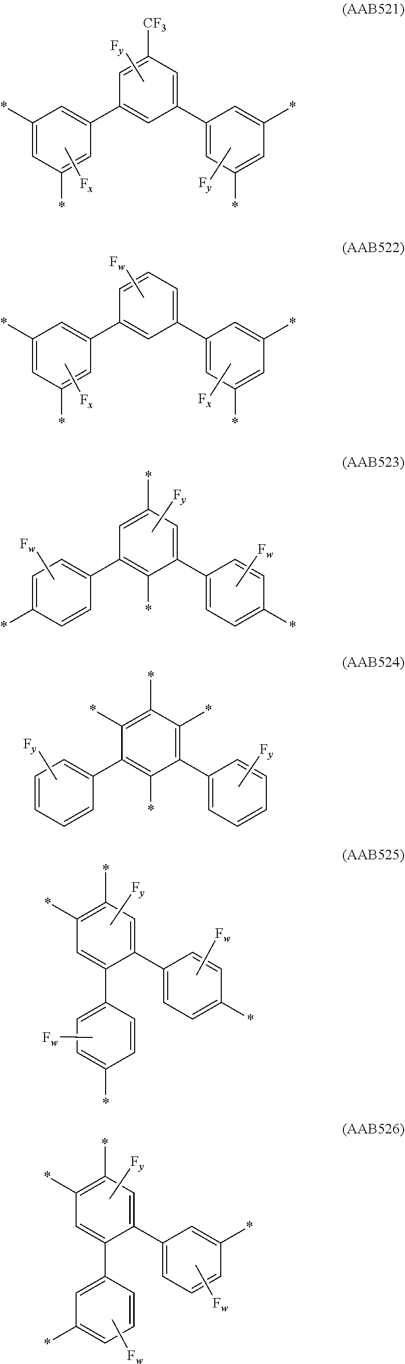

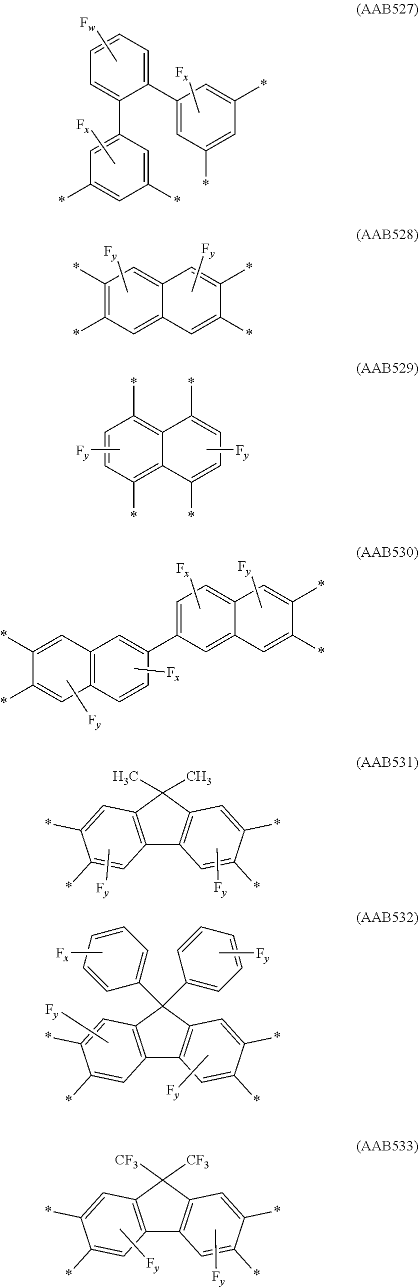

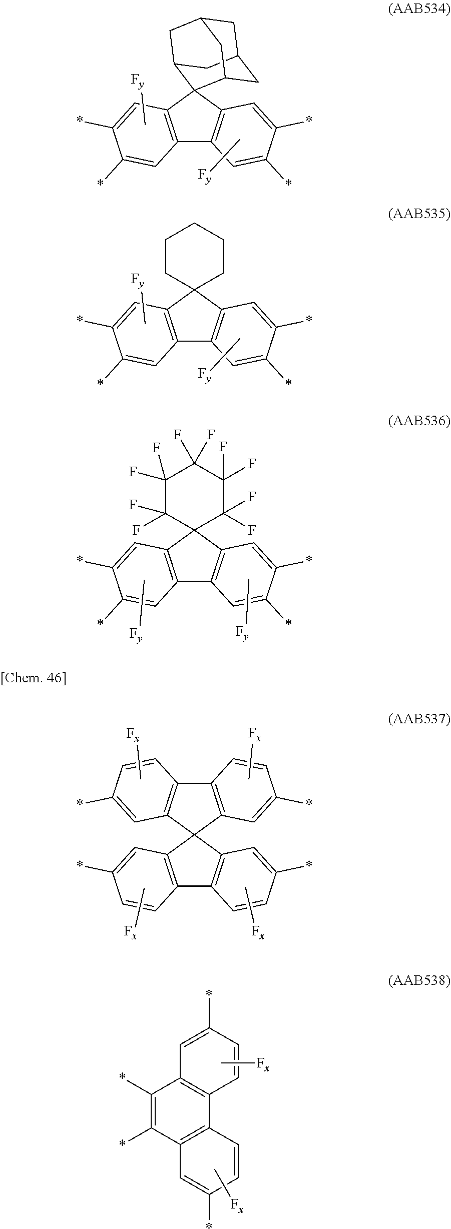

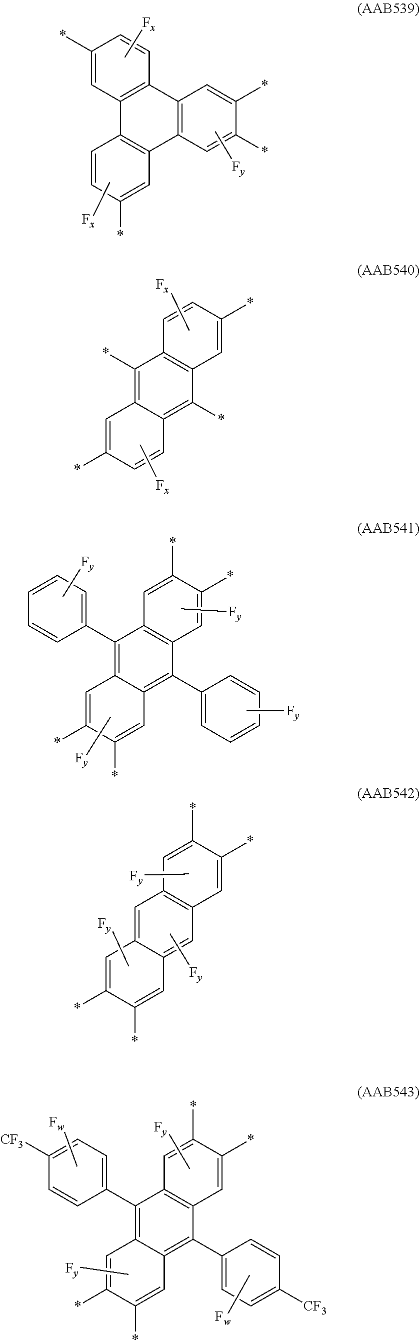

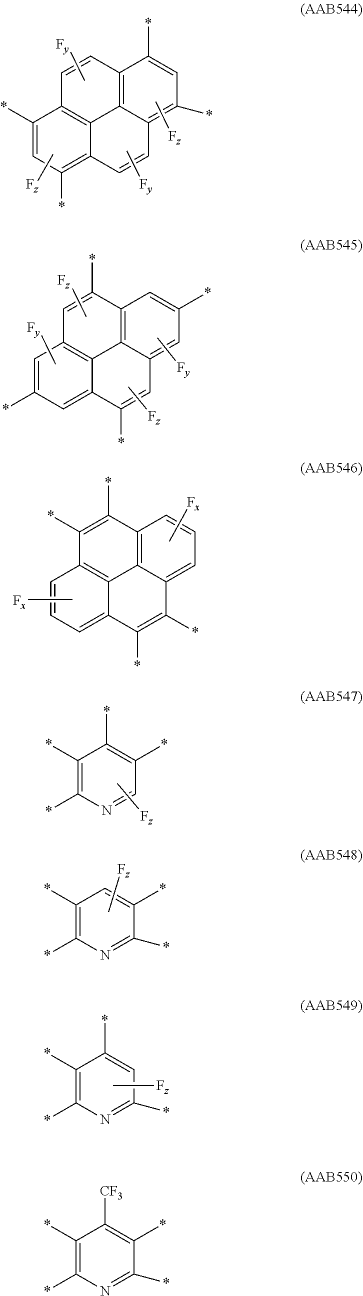

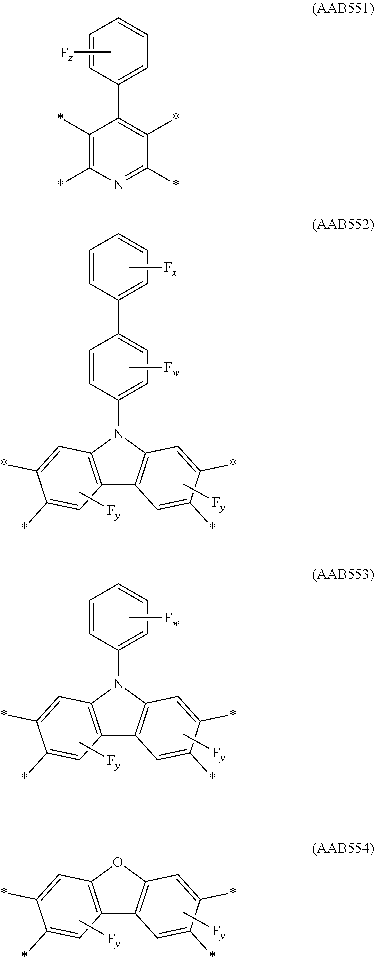

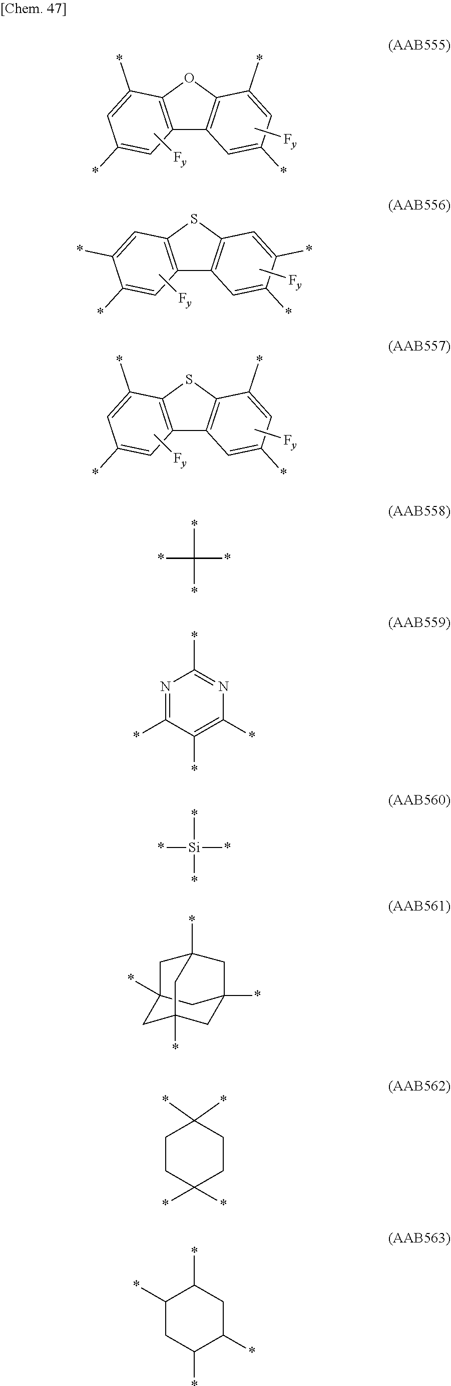

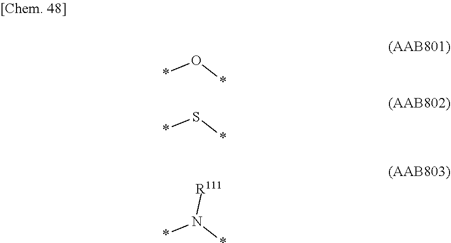

### [R¹¹¹]

R¹¹¹ each independently denotes
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 1 to 18 carbon atoms, or
H, D (deuterium atom) or F (fluorine atom).

Preferably, the monocyclic, linked or condensed aromatic hydrocarbon group represented by R¹¹¹ is a phenyl group or has a structure in which a plurality of benzene rings are linked or condensed.

Preferably, the monocyclic, linked or condensed heteroaromatic group represented by R¹¹¹ has N, O or S as a heteroatom and is a 5-membered ring, a 6-membered ring or a structure in which these or a plurality of benzene rings are condensed.

Preferably, the cyclic heteroaliphatic hydrocarbon group represented by R¹¹¹ has N, O or S as a heteroatom and is a 5-membered ring, a 6-membered ring or a condensed structure thereof.

More preferably, a compound that provides the monocyclic, linked or condensed monovalent aromatic hydrocarbon group represented by R¹¹¹ is benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene,
a compound that provides the monocyclic, linked or condensed monovalent heteroaromatic group is pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene,
a compound that provides the linear, branched or cyclic monovalent aliphatic hydrocarbon group is adamantane, diamantane, cyclohexane or methane, and
a compound that provides the cyclic monovalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

Still more preferably, the monocyclic, linked or condensed aromatic hydrocarbon group represented by R¹¹¹ is phenyl, biphenyl, terphenyl, naphthalene, fluorene, spirobifluorene, 9,9-dimethylfluorene, 9,9-diphenylfluorene, benzofluorene, phenanthrene, fluoranthene or triptycene,
the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole or benzothiazole,
the linear, branched or cyclic aliphatic hydrocarbon group is adamantane, diamantane, cyclohexane or methane, and
a compound that provides the cyclic heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

Still more preferably, the monocyclic, linked or condensed aromatic hydrocarbon group represented by R¹¹¹ is phenyl, biphenyl, terphenyl, naphthalene, fluorene, spirobifluorene, 9,9-dimethylfluorene, 9,9-diphenylfluorene, benzofluorene, phenanthrene, fluoranthene or triptycene,
the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole or benzothiazole,
the linear, branched or cyclic aliphatic hydrocarbon group is adamantane, diamantane or cyclohexane, and
a compound that provides the cyclic heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane or 1,4,7,10-tetraazacyclododecane.

When R¹¹¹ denotes a substituted aromatic hydrocarbon group, a substituted heteroaromatic group, a substituted aliphatic hydrocarbon group or a substituted heteroaliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a hydrogen atom, a fluorine atom, a deuterium atom and a structure represented by the formula (001), and
a combination of these groups.

A substituent of R¹¹¹ is preferably each independently substituted with one or more groups selected from the group consisting of
a methyl group, a trifluoromethyl group, a trifluoromethoxy group, an alkyl group with 2 to 10 carbon atoms, a cyano group, a deuterium atom, a fluorine atom, a phenyl group, a biphenylyl group, a naphthyl group, a phenanthryl group, a pyridyl group, a pyrimidyl group, a pyrazyl group, a triazyl group, an indolyl group, a carbazolyl group, a benzothienyl group, a dibenzothienyl group, a benzofuranyl group, a dibenzofuranyl group, a thiazolyl group, a benzothiazolyl group and a structure represented by the formula (001), and
a combination of these groups.

A substituent of R¹¹¹ is more preferably each independently substituted with one or more groups selected from the group consisting of
a methyl group, a trifluoromethyl group, a trifluoromethoxy group, an alkyl group with 2 to 10 carbon atoms, a fluorine atom, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group, a thiazolyl group and a structure represented by the formula (001), and
a combination of these groups.

A substituent of R¹¹¹ is still more preferably each independently
(vii) a methyl group, a methoxy group, an alkyl group with 2 to 10 carbon atoms, an alkoxy group with 2 to 10 carbon atoms, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group or a thiazolyl group,
(viii) the group represented by (vii) substituted with one or more groups selected from the group consisting of a fluorine atom, a perfluoroalkyl group with 1 to 10 carbon atoms and a perfluoroalkoxy group with 1 to 10 carbon atoms, or
(ix) a fluorine atom or a structure represented by the formula (001).

A substituent of R¹¹¹ is still more preferably each independently
(vii′) a methyl group, an ethyl group, a butyl group, a methoxy group, an ethoxy group, a butoxy group, a phenyl group, a naphthyl group, a pyridyl group, a pyrimidyl group, a pyrazyl group, a triazyl group, a carbazolyl group, a dibenzothienyl group, a dibenzofuranyl group or a thiazolyl group,
(viii′) the group represented by (vii′) substituted with one or more groups selected from the group consisting of a fluorine atom, a perfluoroalkyl group with 1 to 6 carbon atoms and a perfluoroalkoxy group with 1 to 6 carbon atoms, or
(ix′) a fluorine atom or a structure represented by the formula (001).

A substituent of R¹¹¹ is preferably each independently
a trifluoromethyl group, a trifluoromethoxy group, an alkyl group having 2 to 10 carbon atoms and substituted with 3 or more fluorine atoms, an alkoxy group having 2 to 10 carbon atoms and substituted with 3 or more fluorine atoms, a fluorine atom or a structure represented by the formula (001).

### Specific Example of R¹¹¹

R¹¹¹ may have, for example, the structures represented above by (AAA1) to (AAA126) and the structures represented below by (AAC1) to (AAC450). The mark “*” in the following structures denotes a binding site.

F denotes a fluorine atom, v denotes an integer in the range of 0 to 5, w denotes an integer in the range of 0 to 4, x denotes an integer in the range of 0 to 3, y denotes an integer in the range of 0 to 2, and z denotes an integer of 0 or 1.

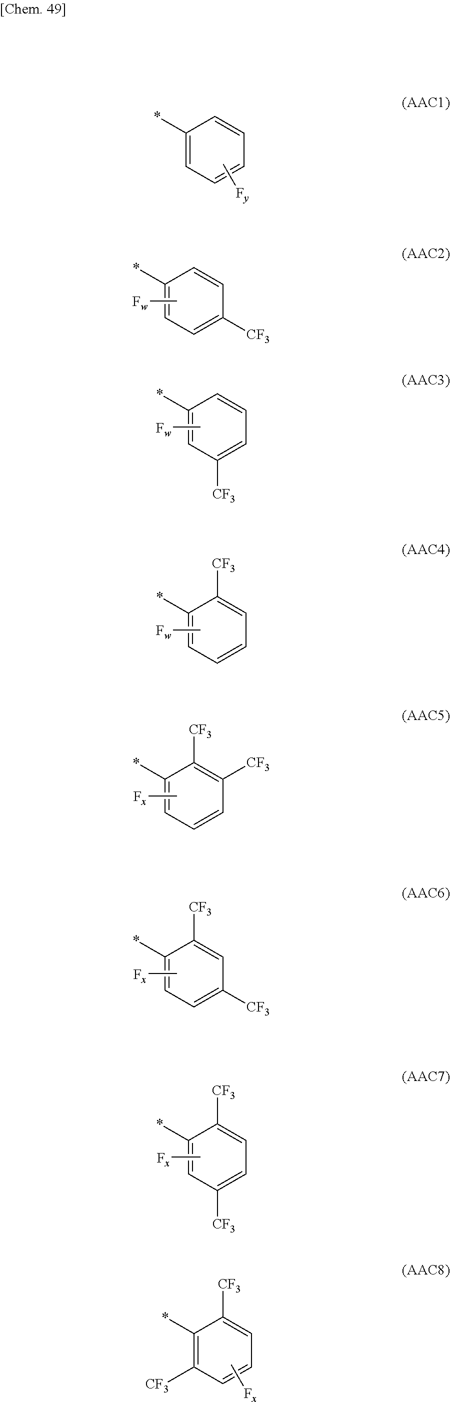

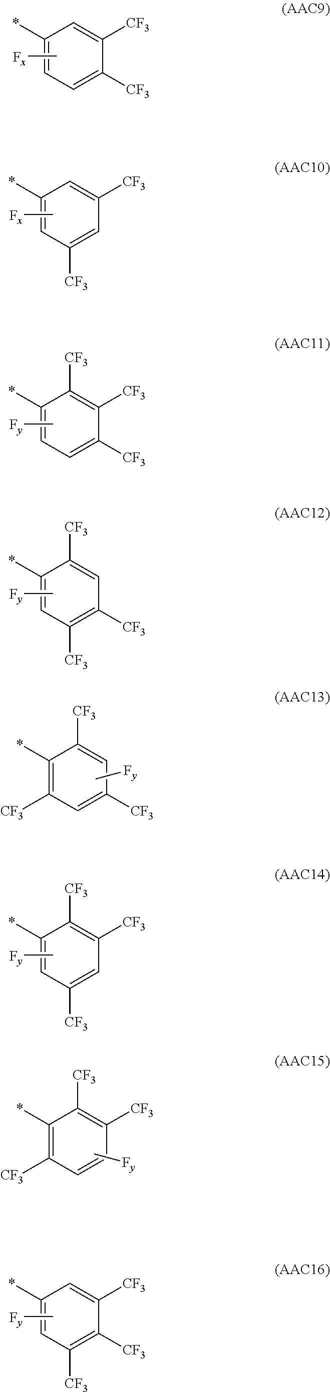

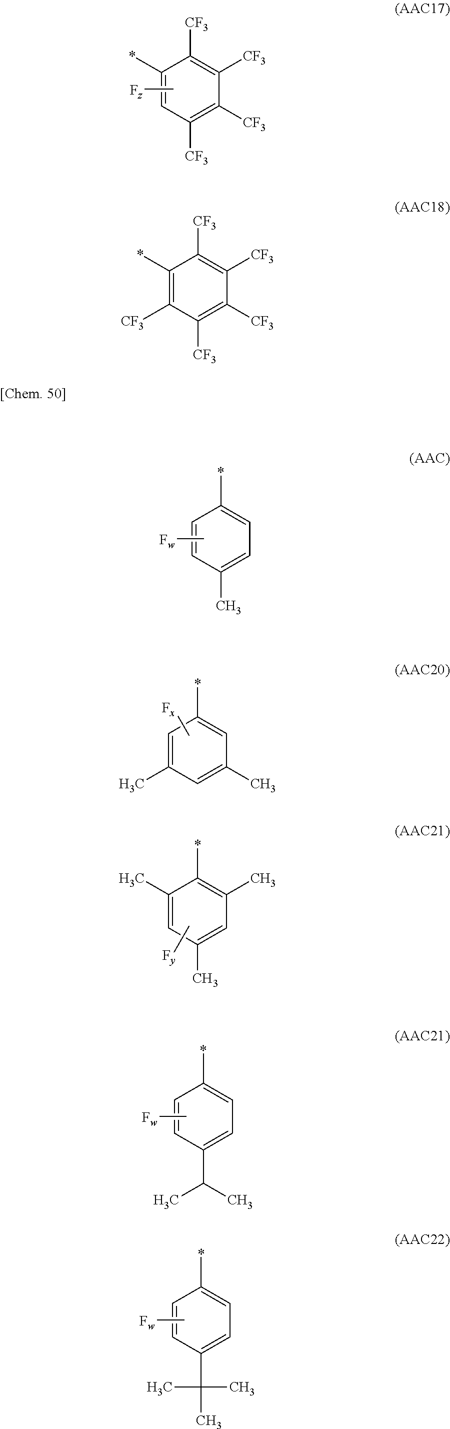

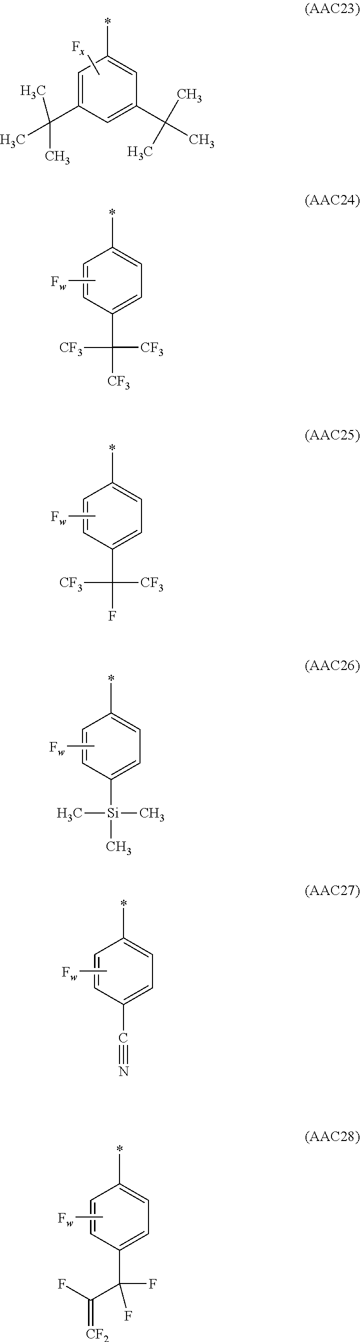

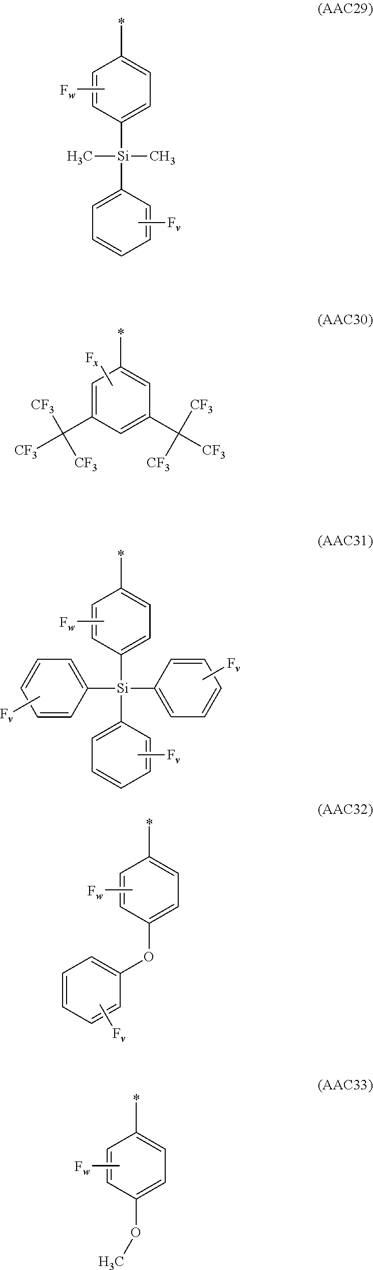

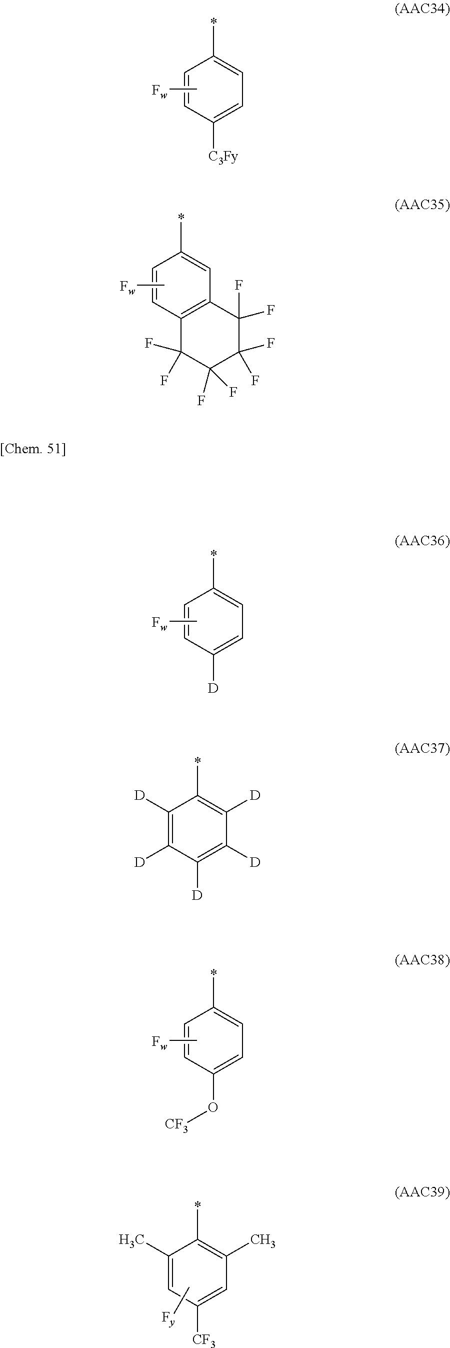

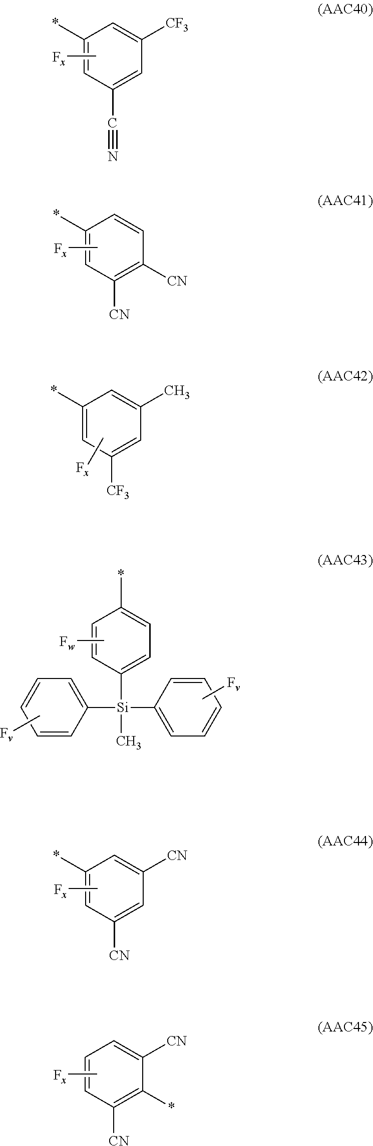

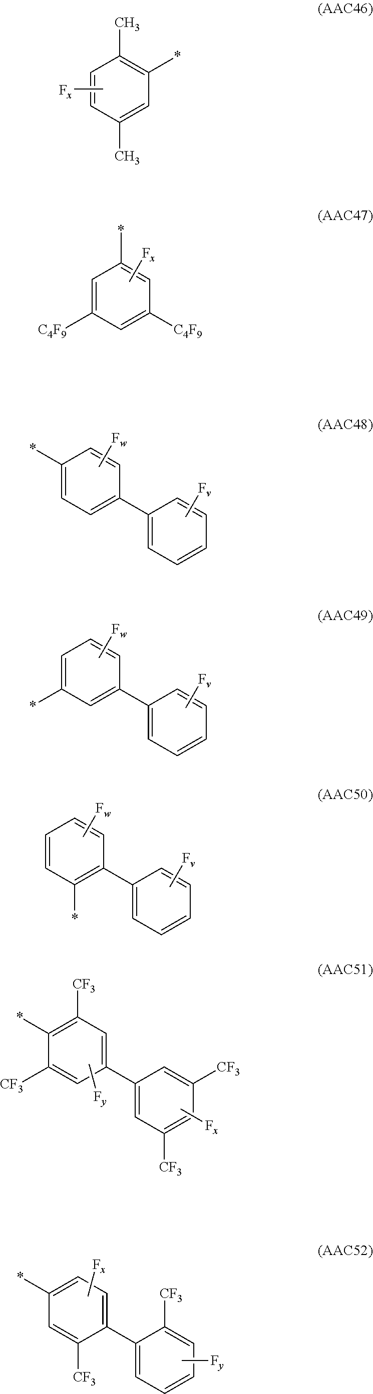

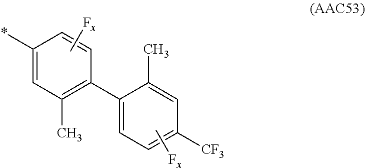

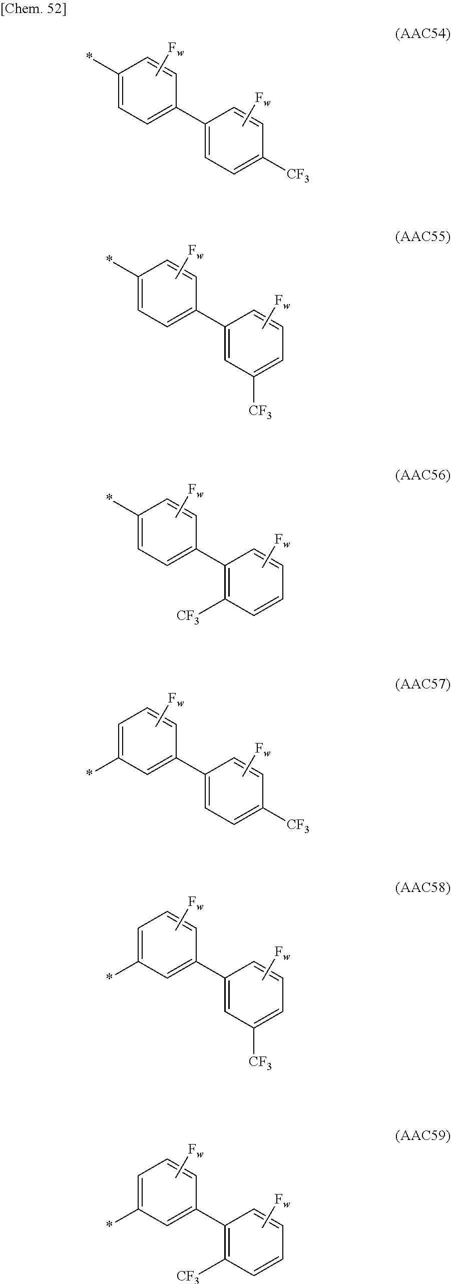

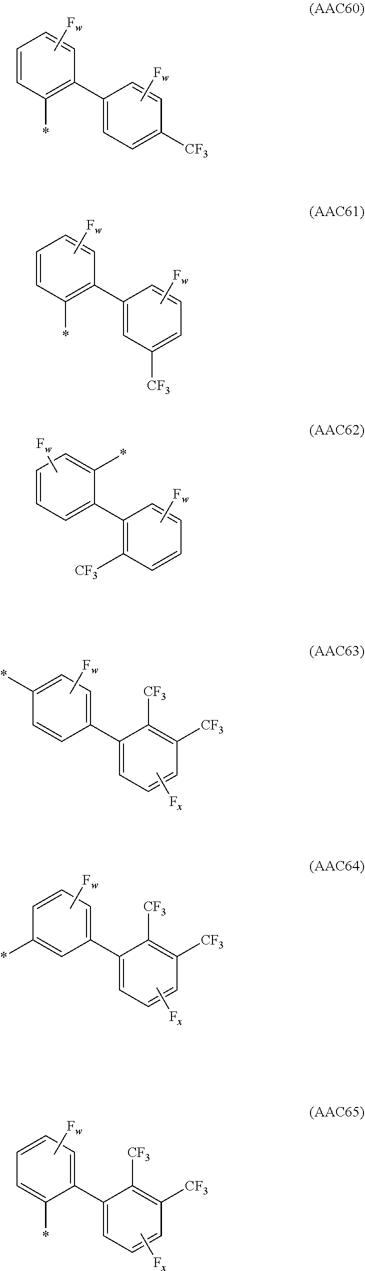

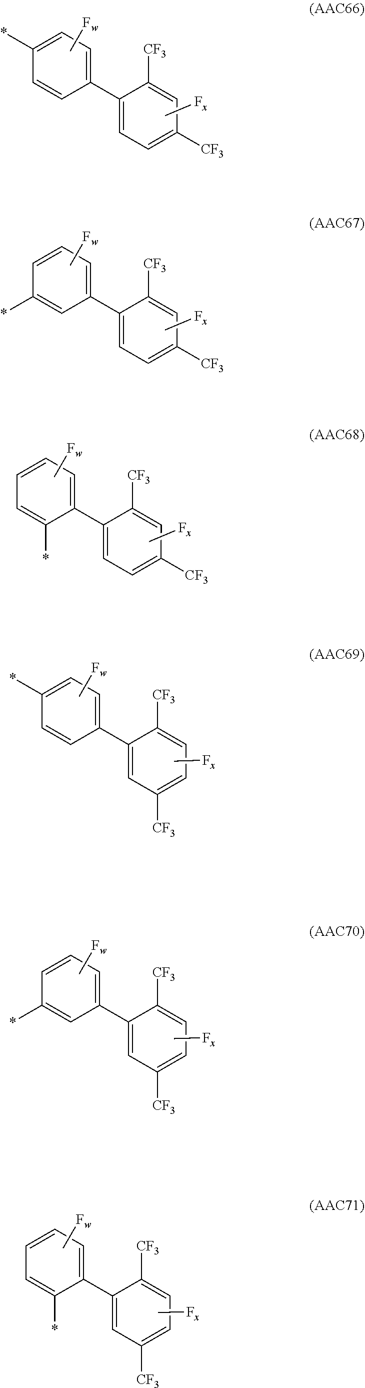

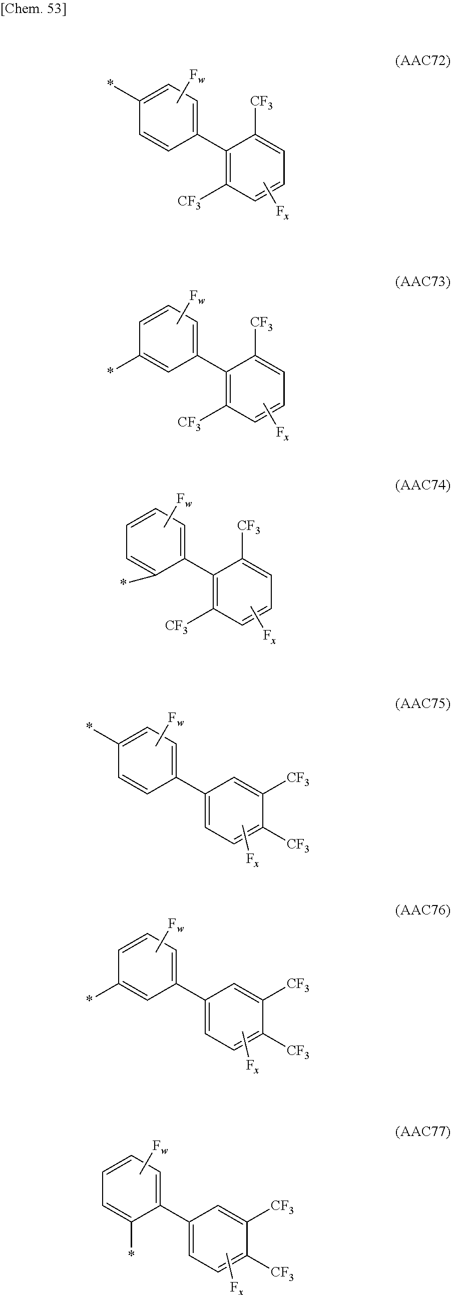

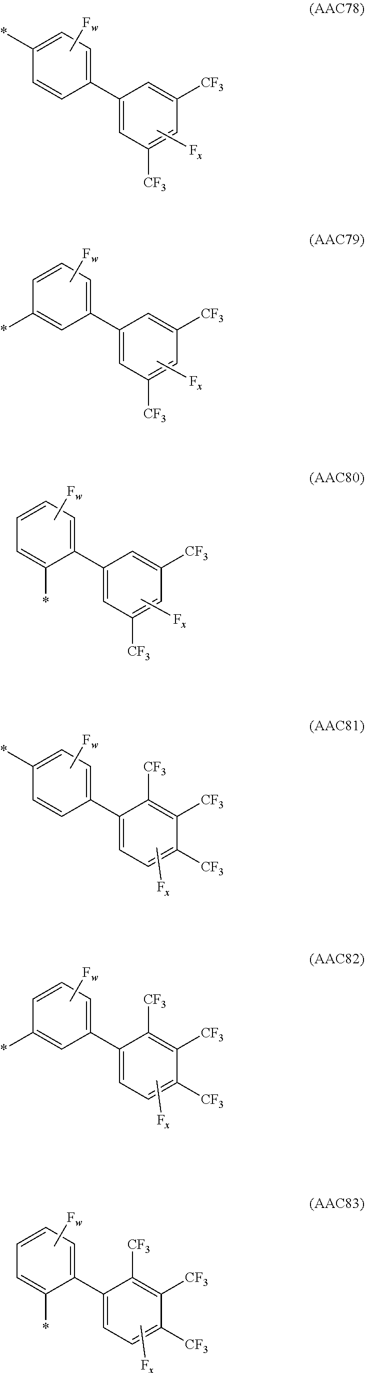

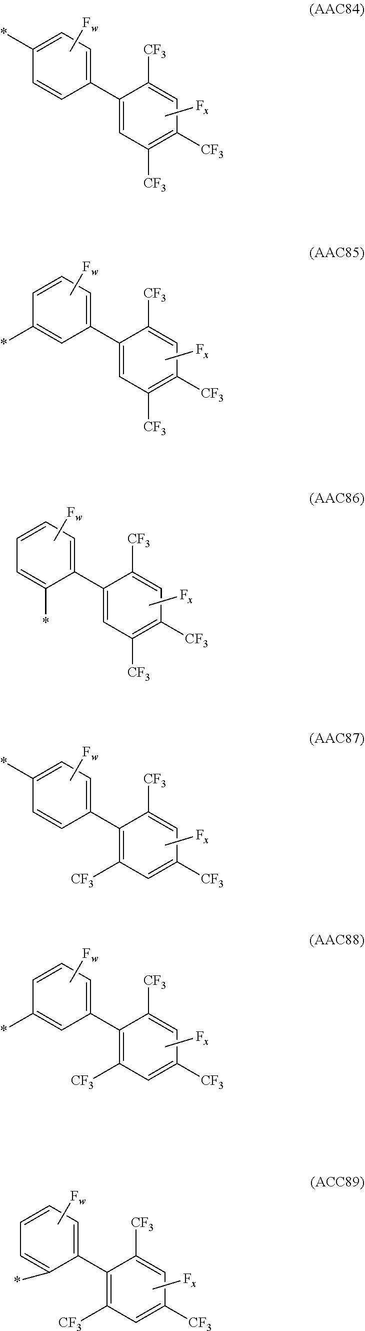

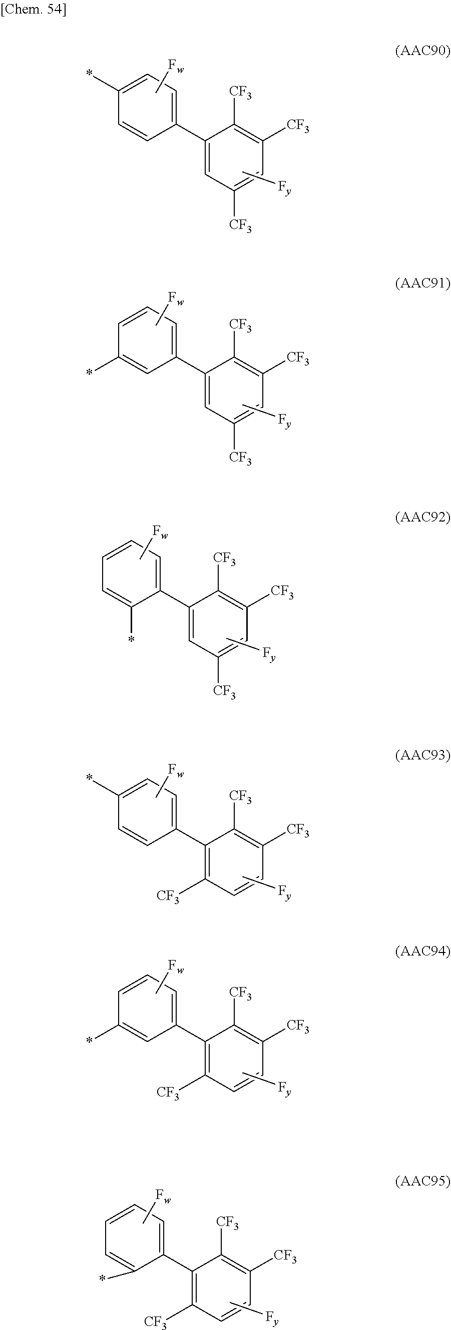

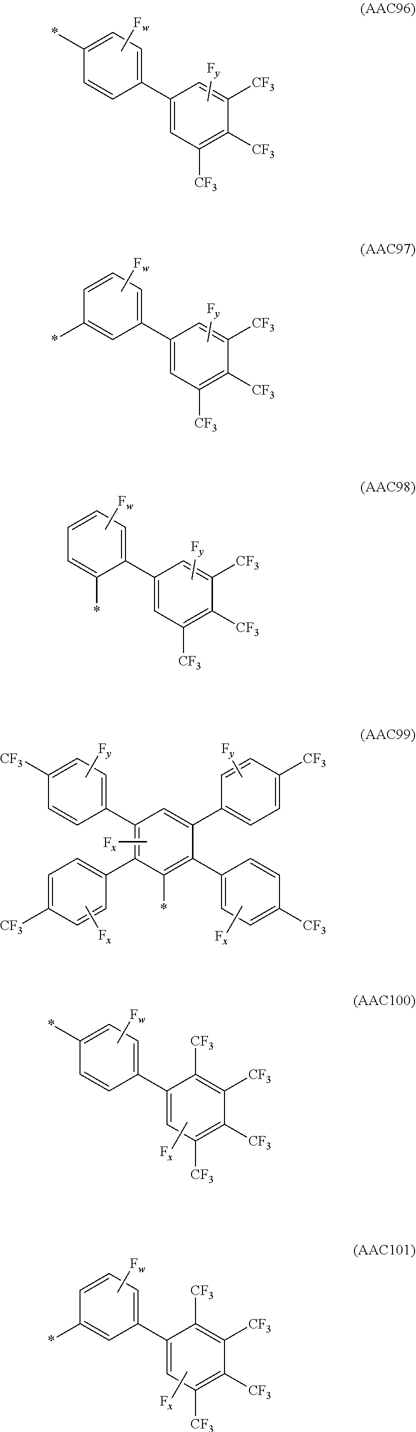

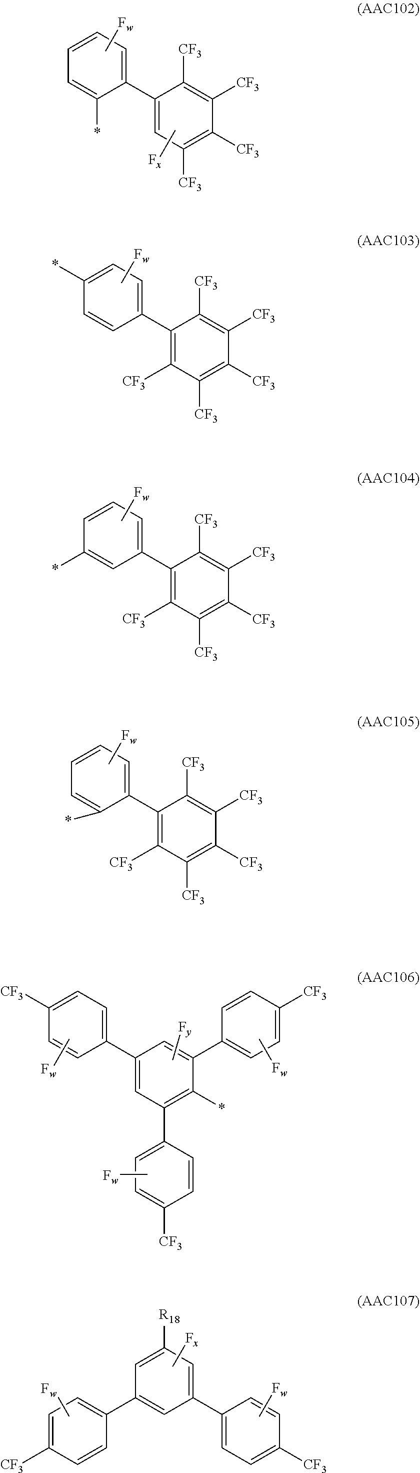

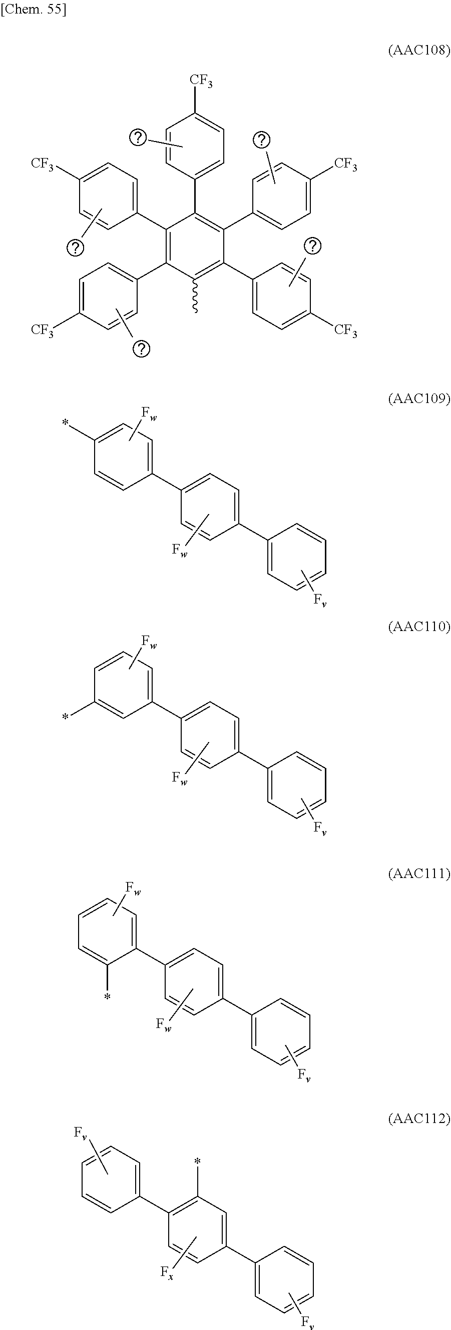

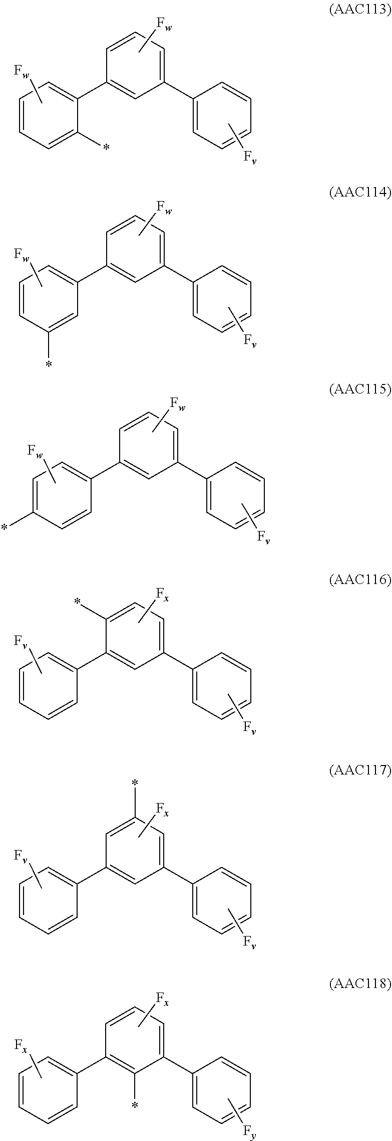

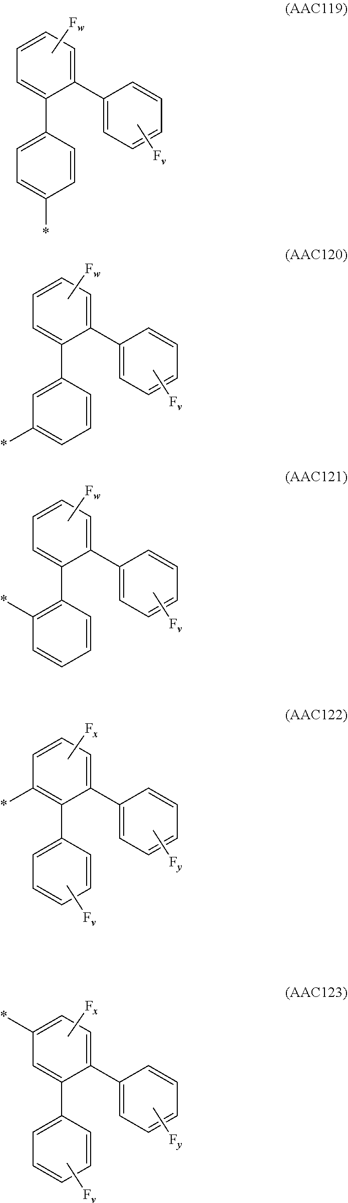

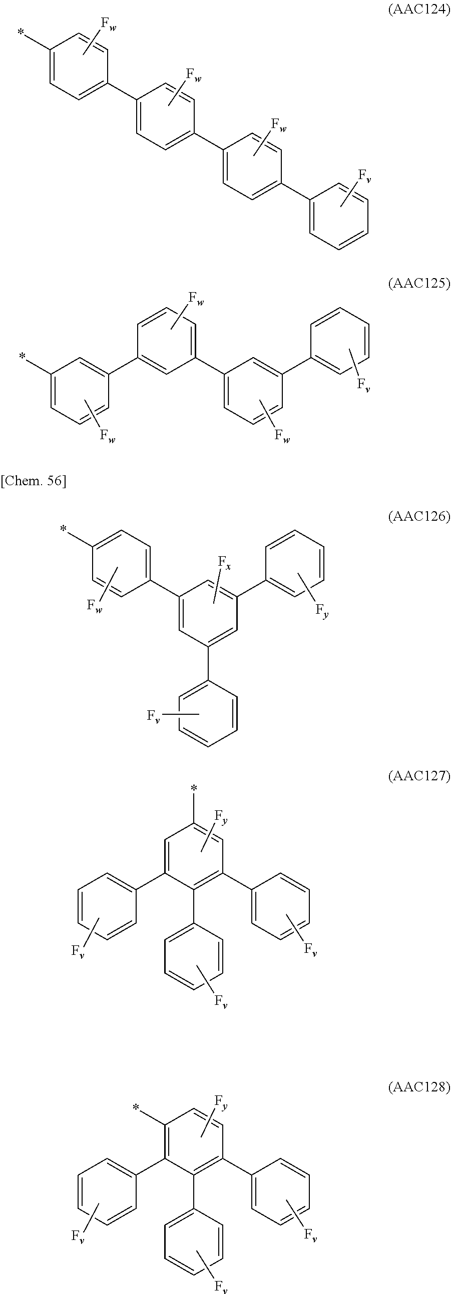

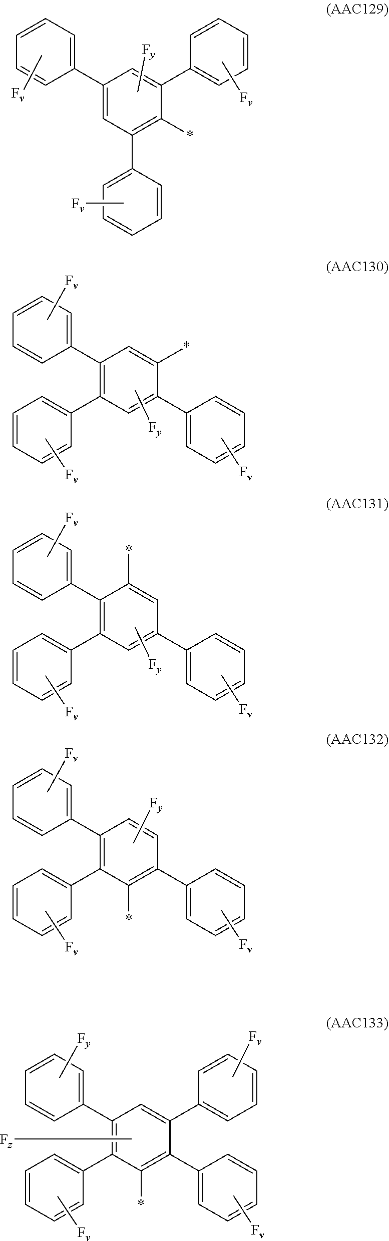

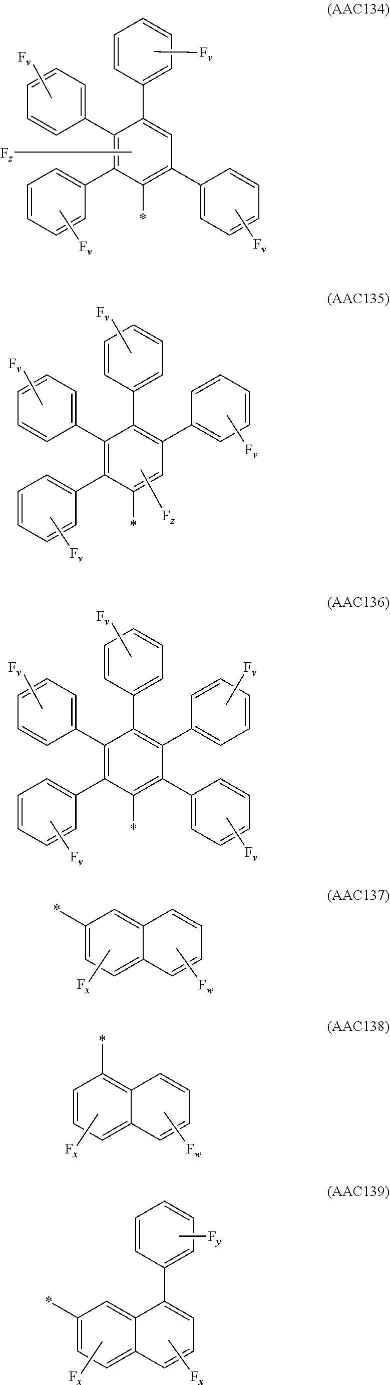

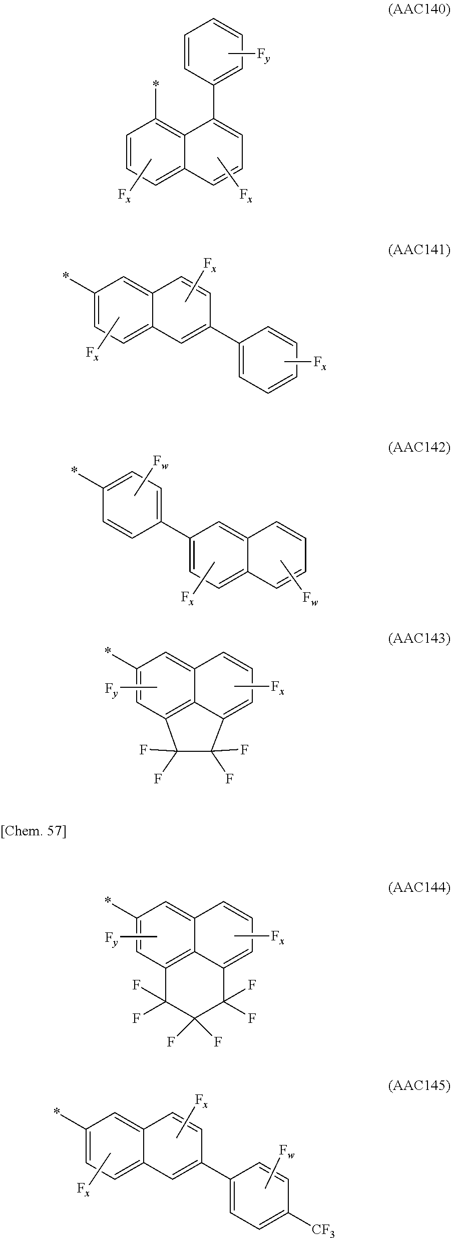

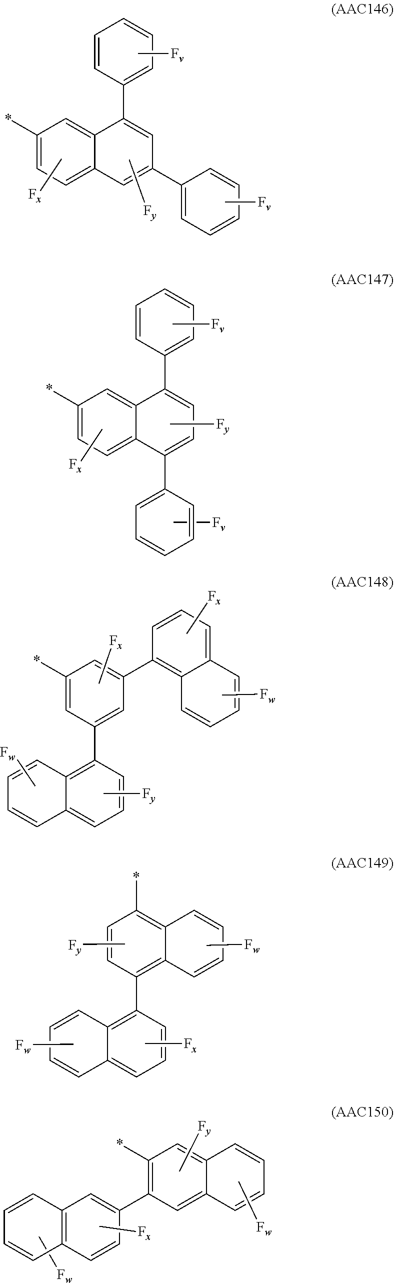

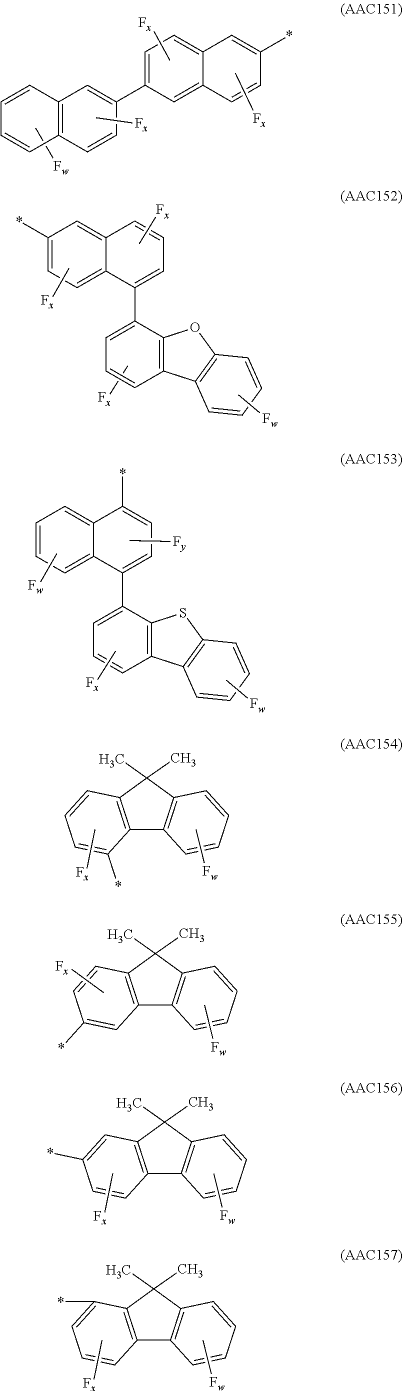

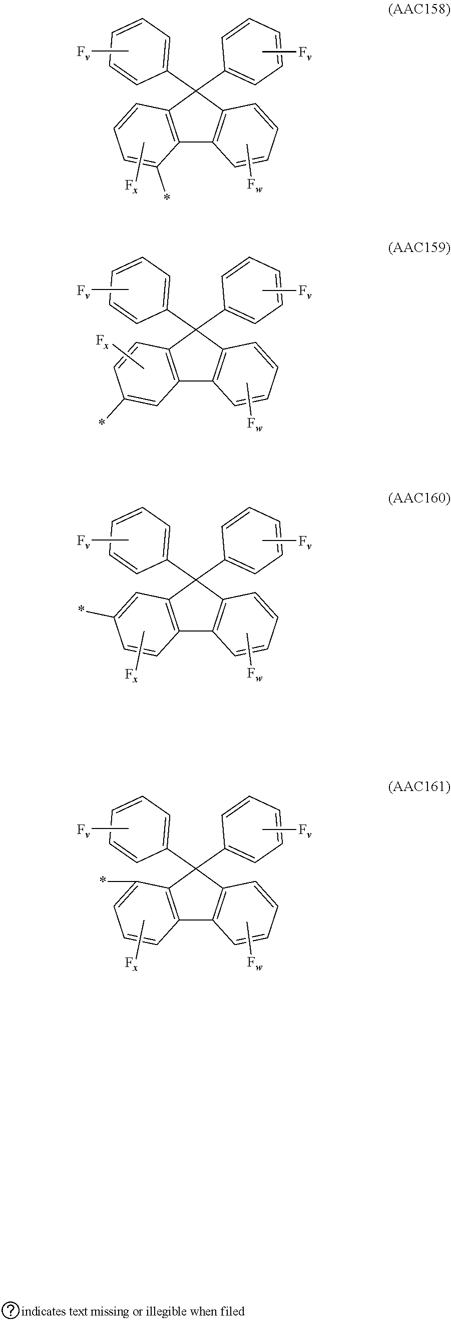

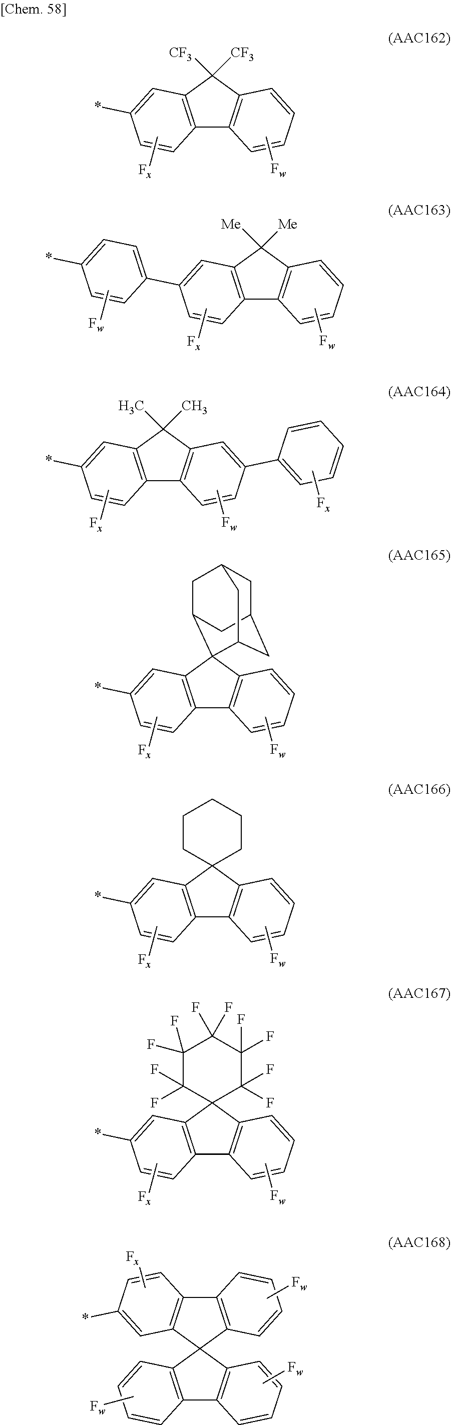

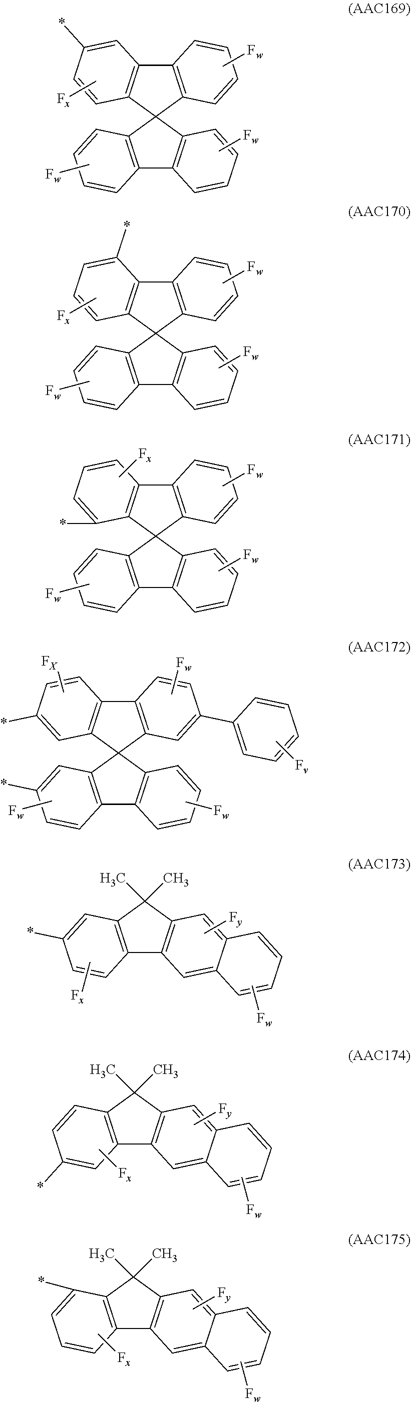

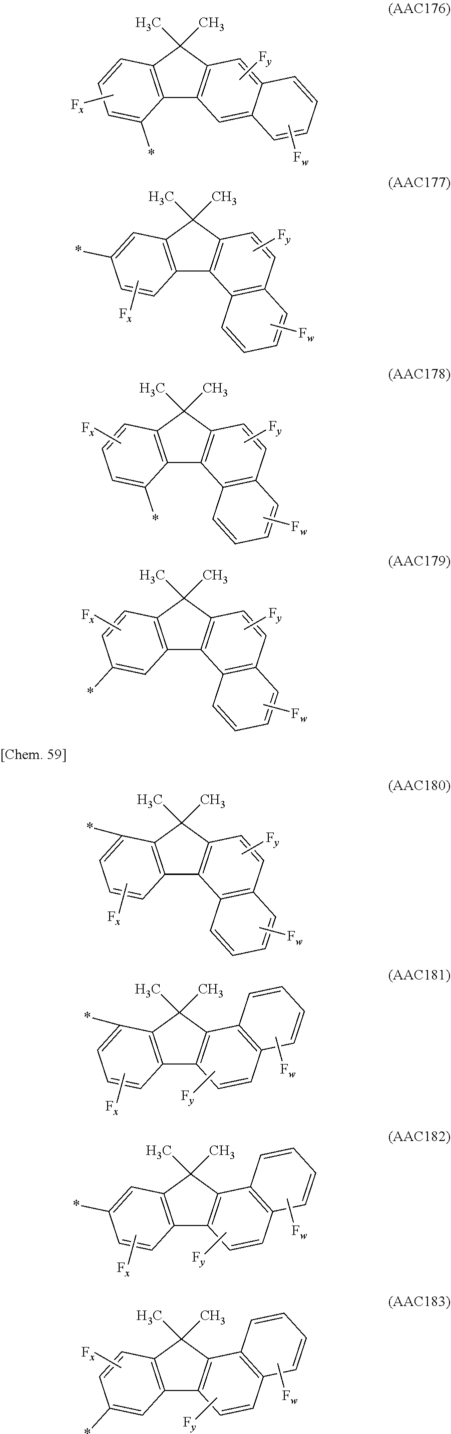

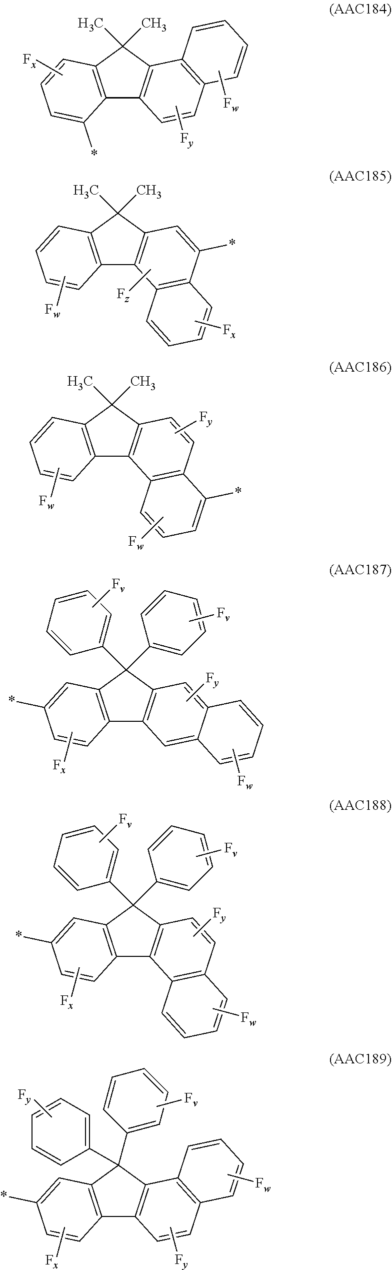

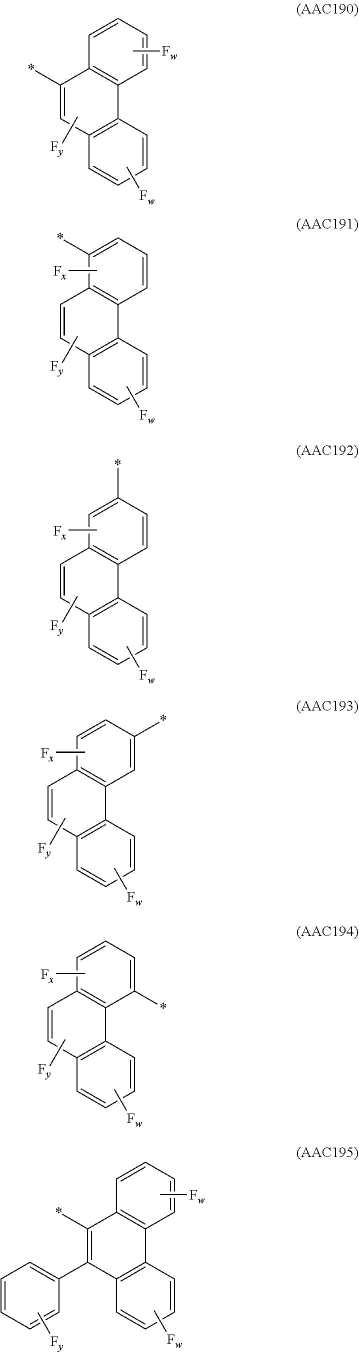

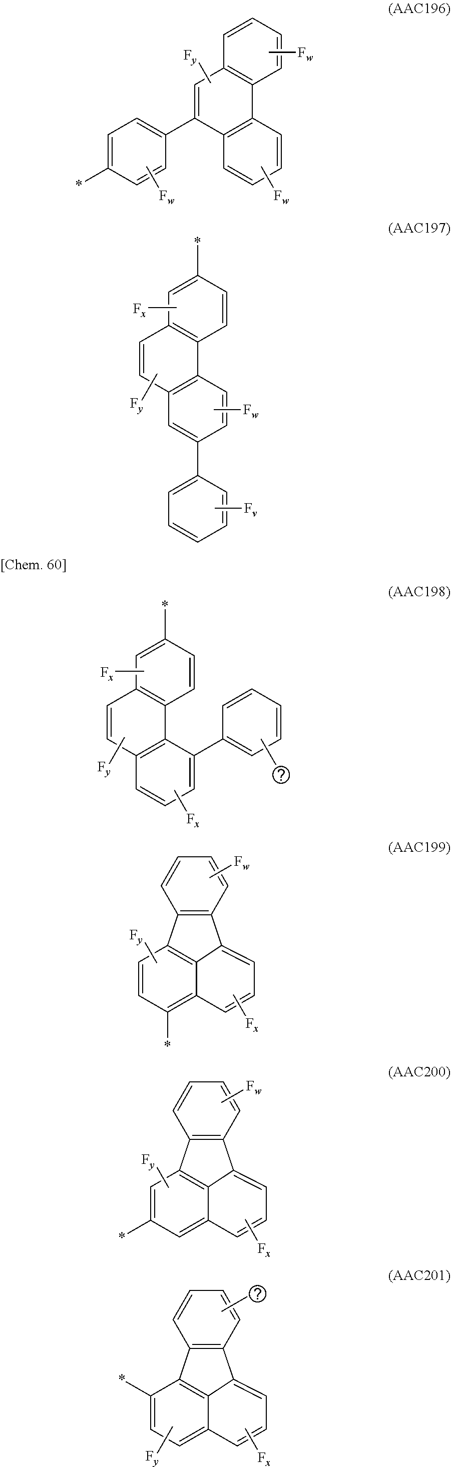

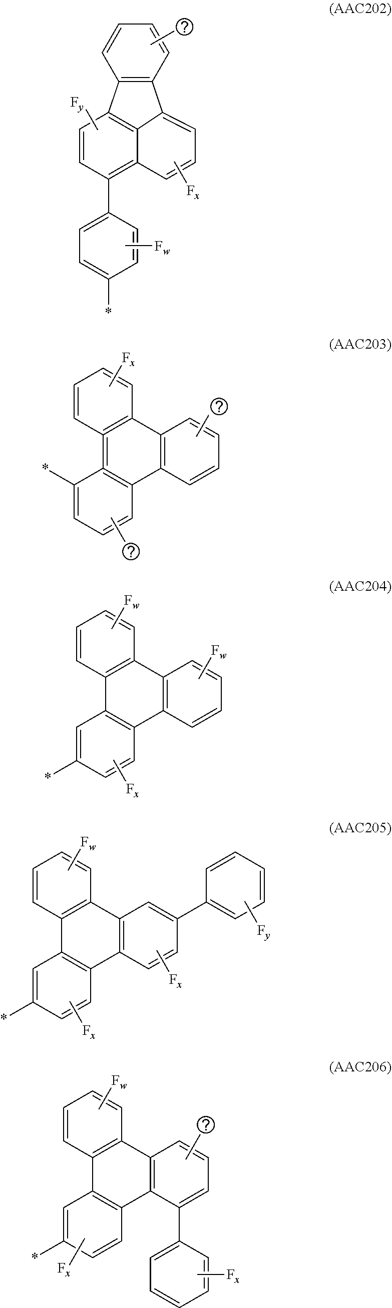

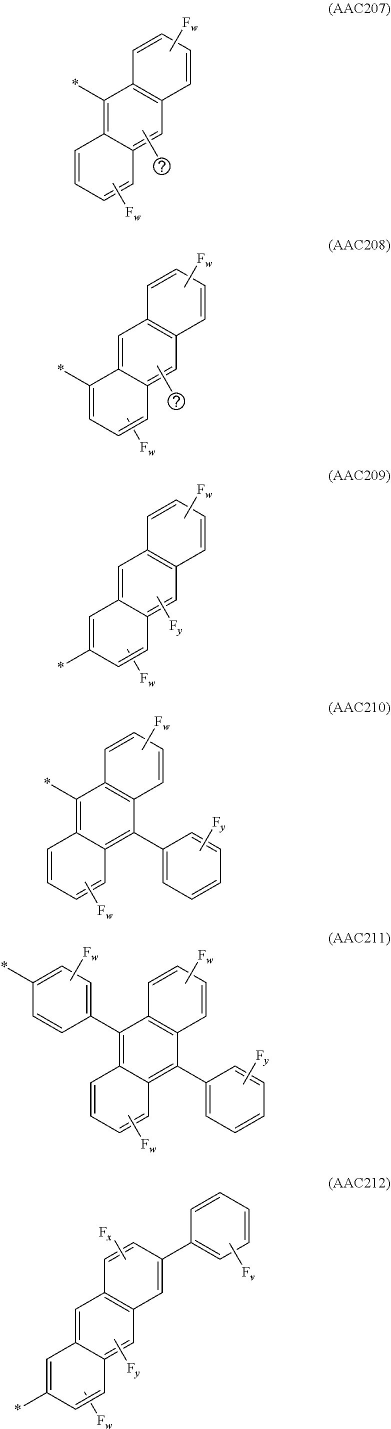

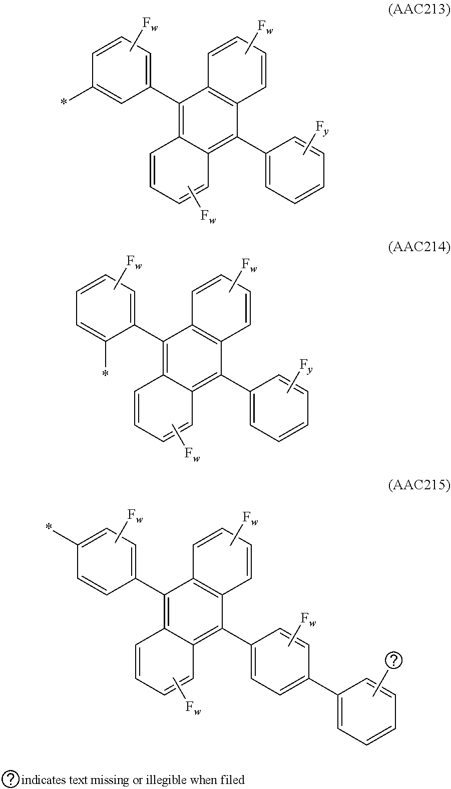

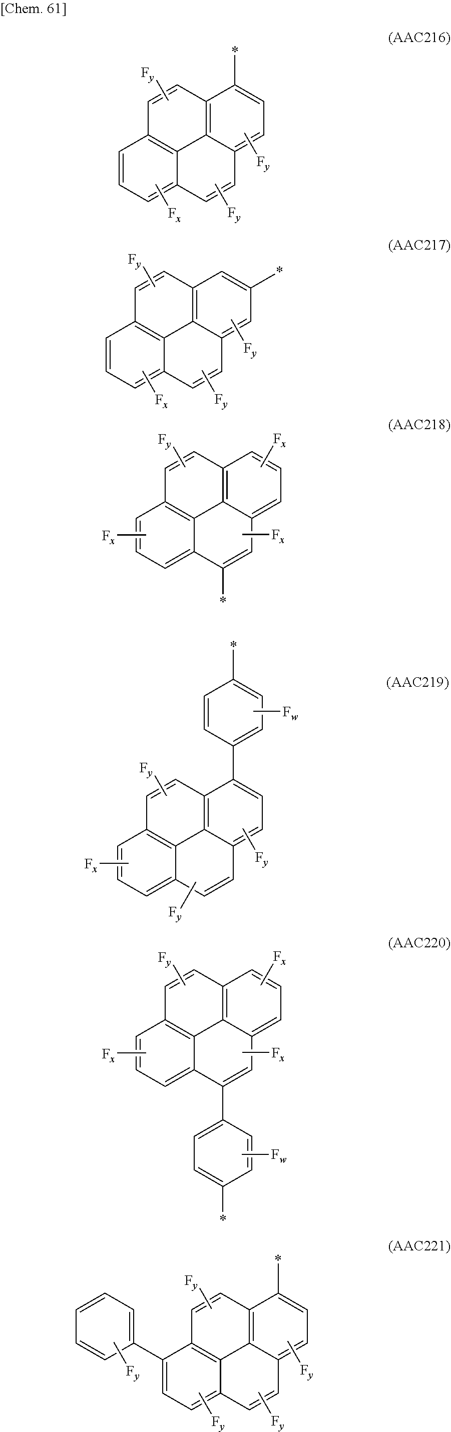

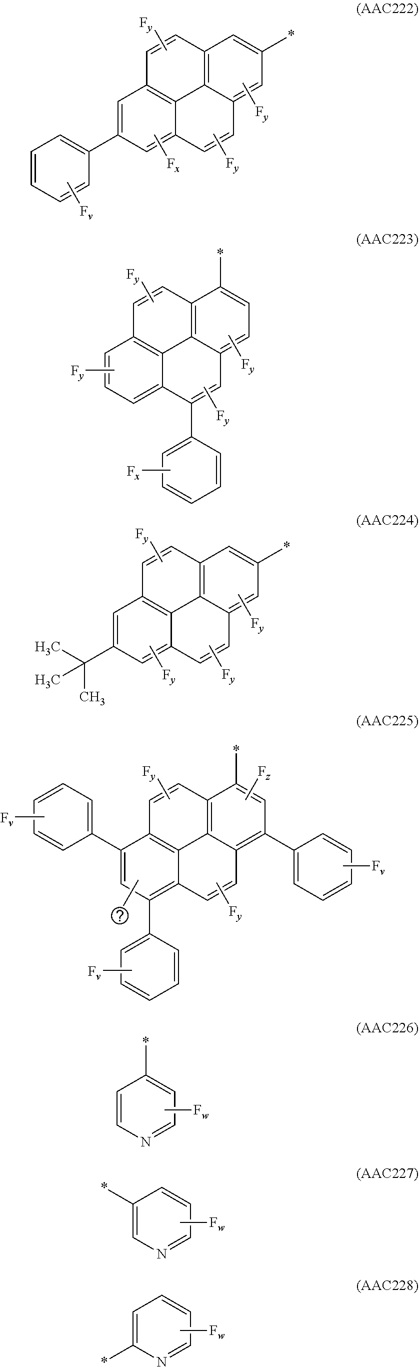

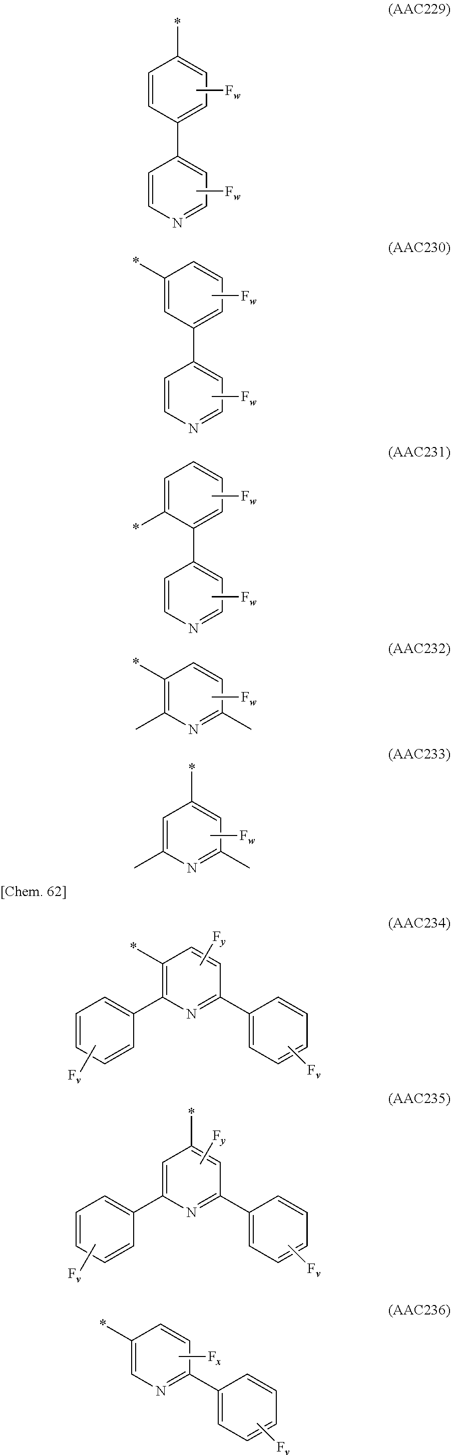

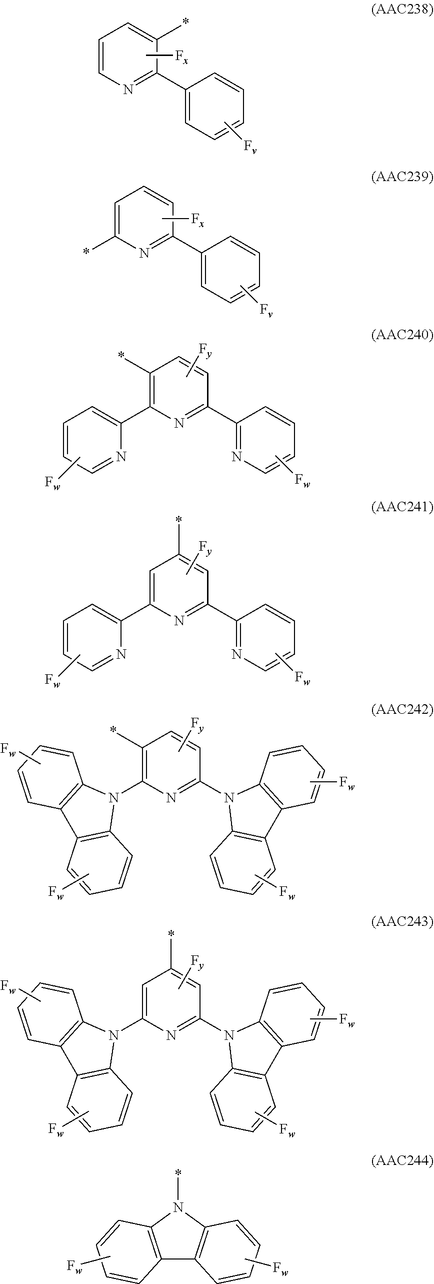

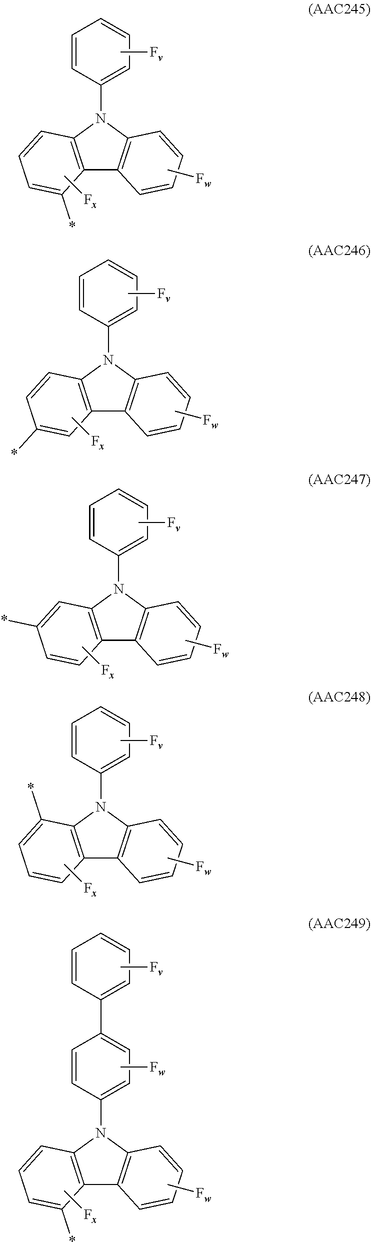

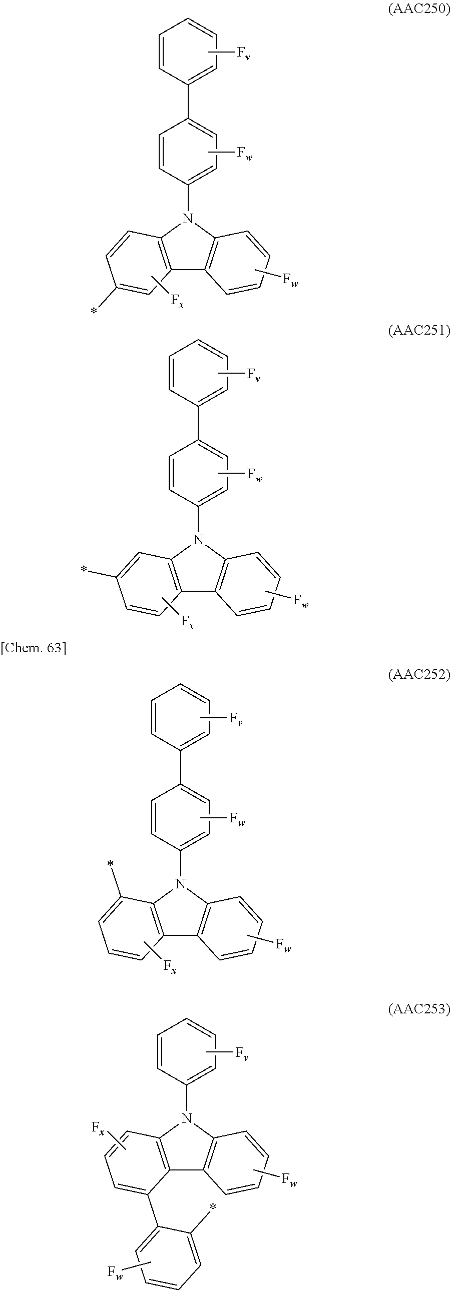

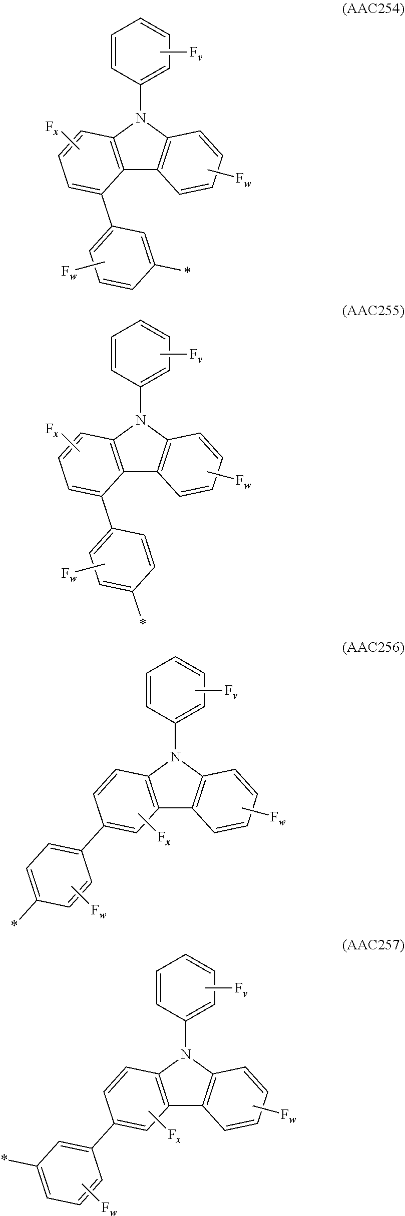

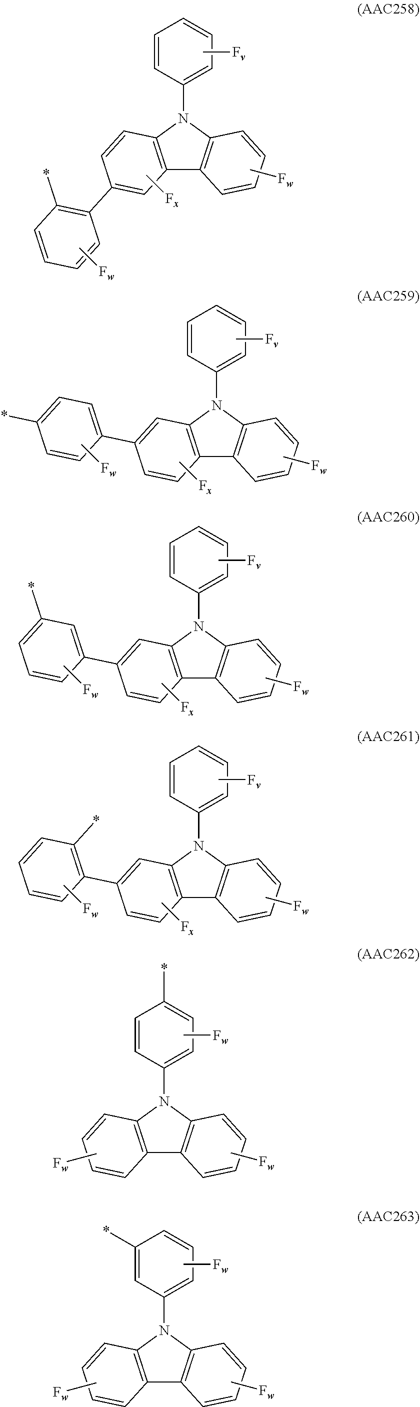

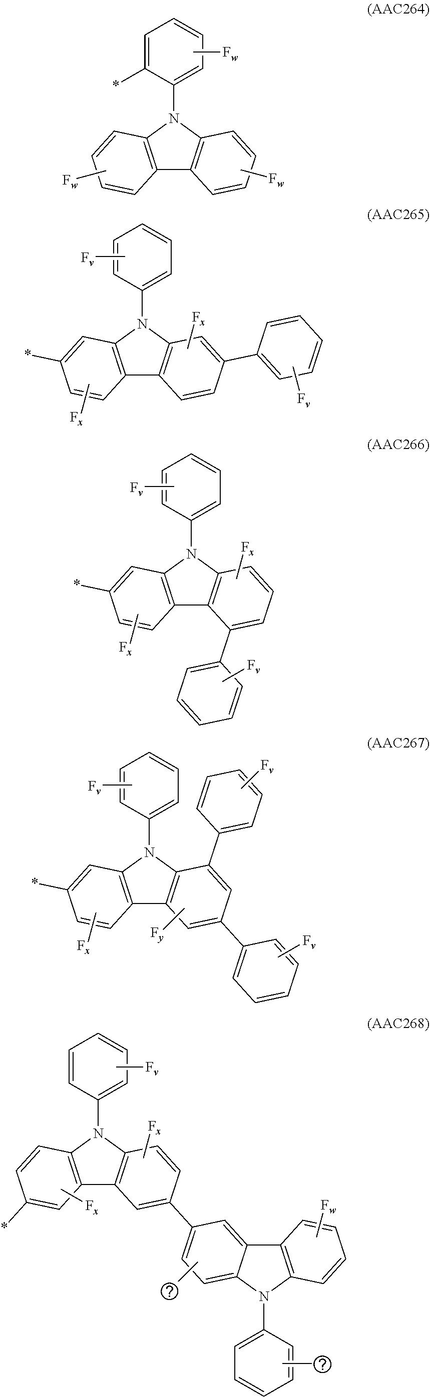

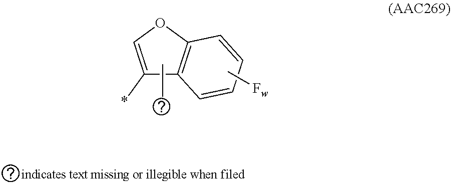

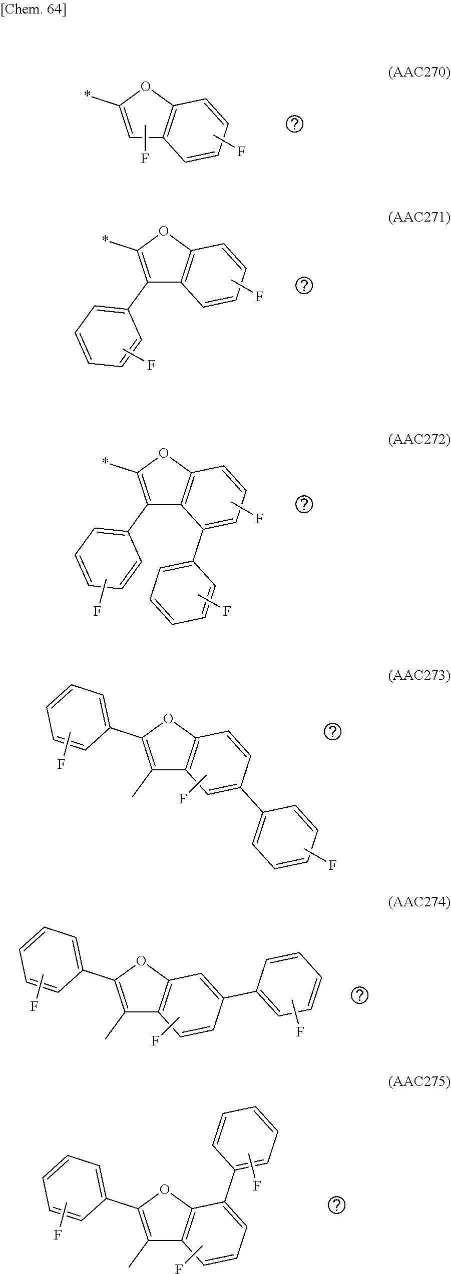

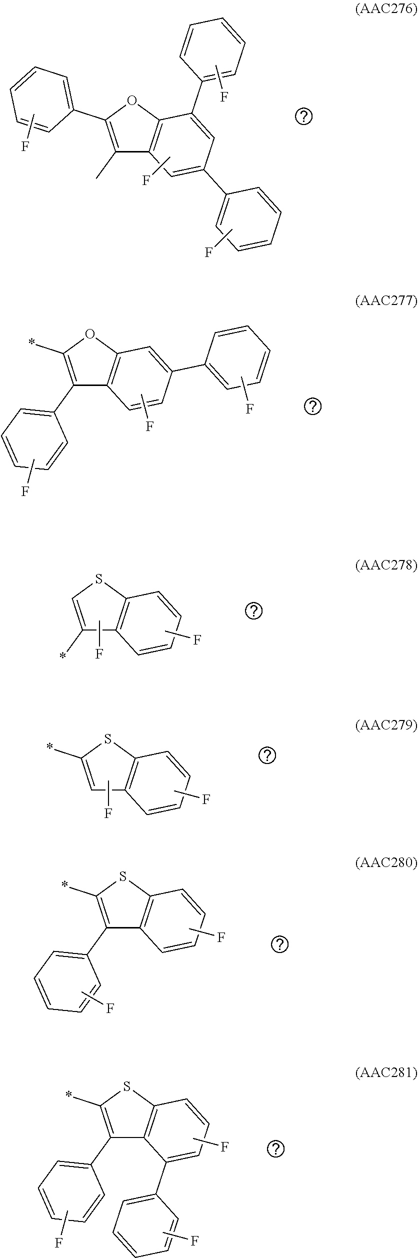

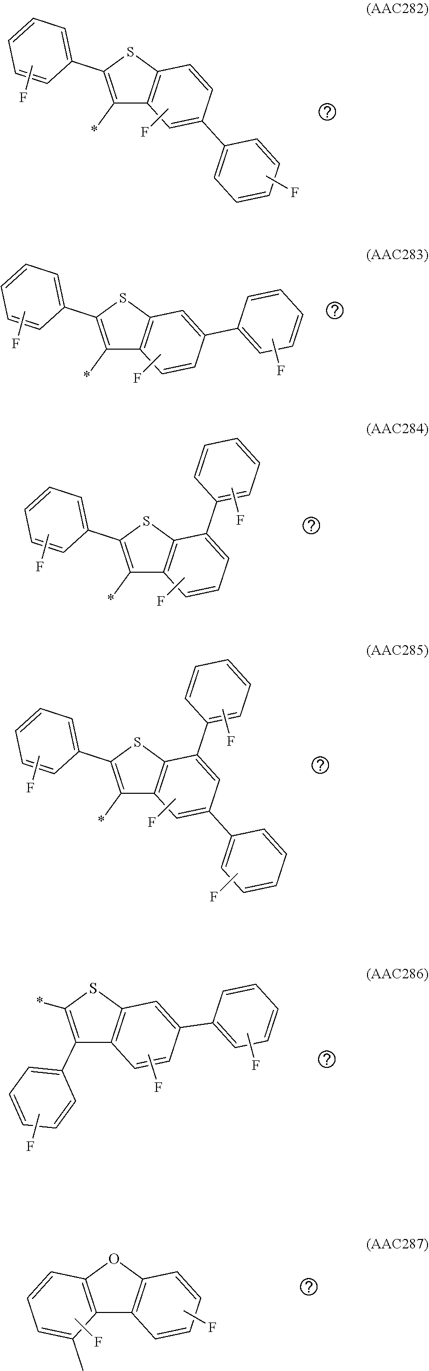

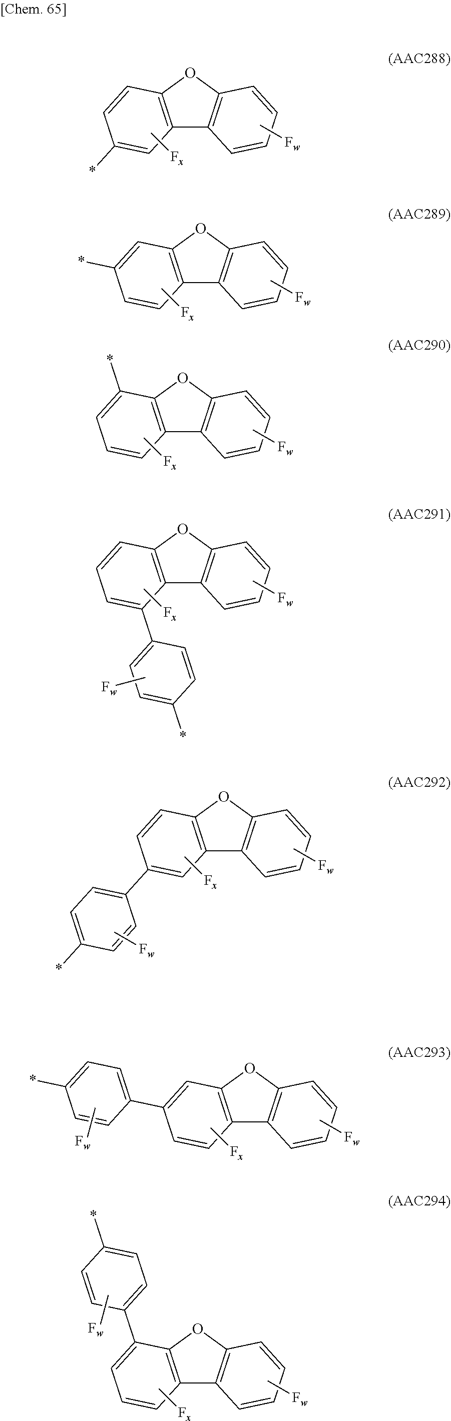

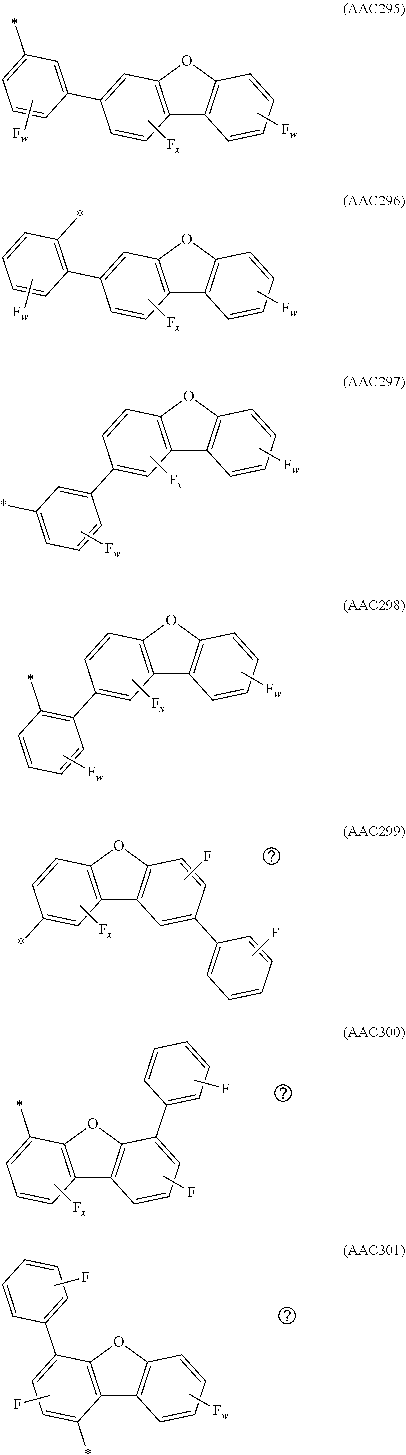

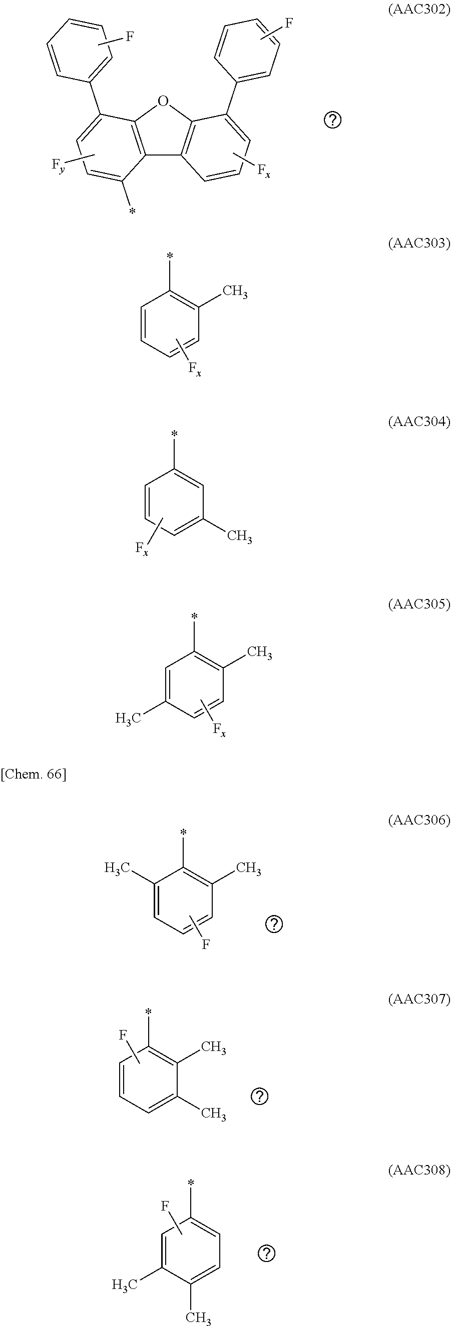

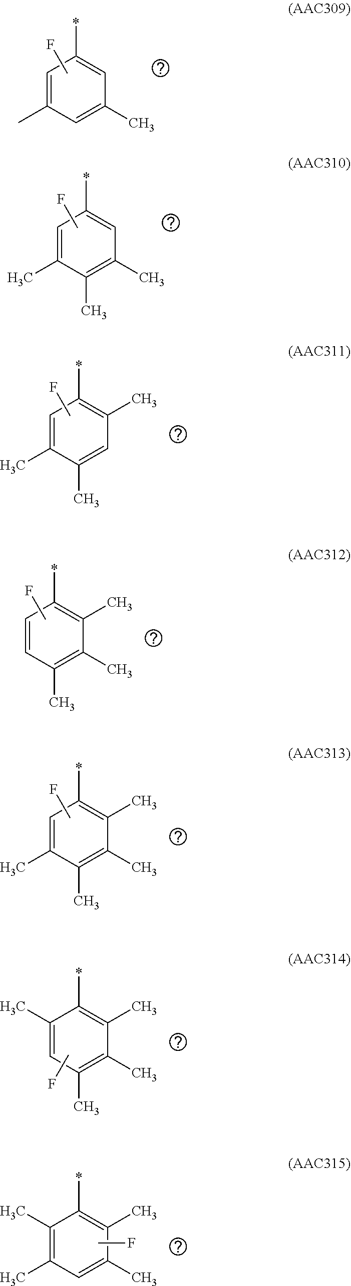

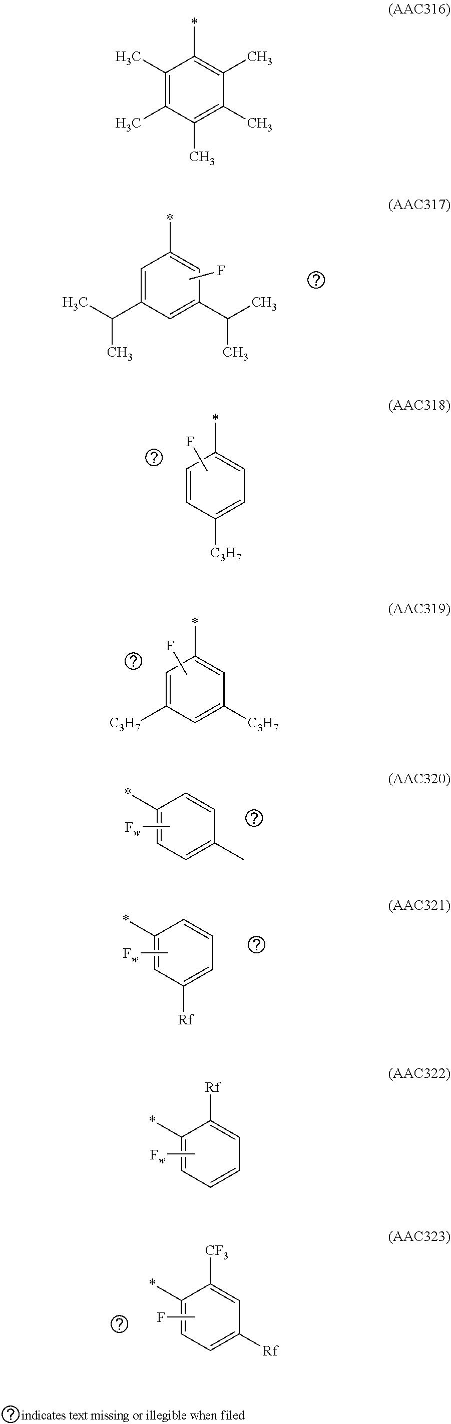

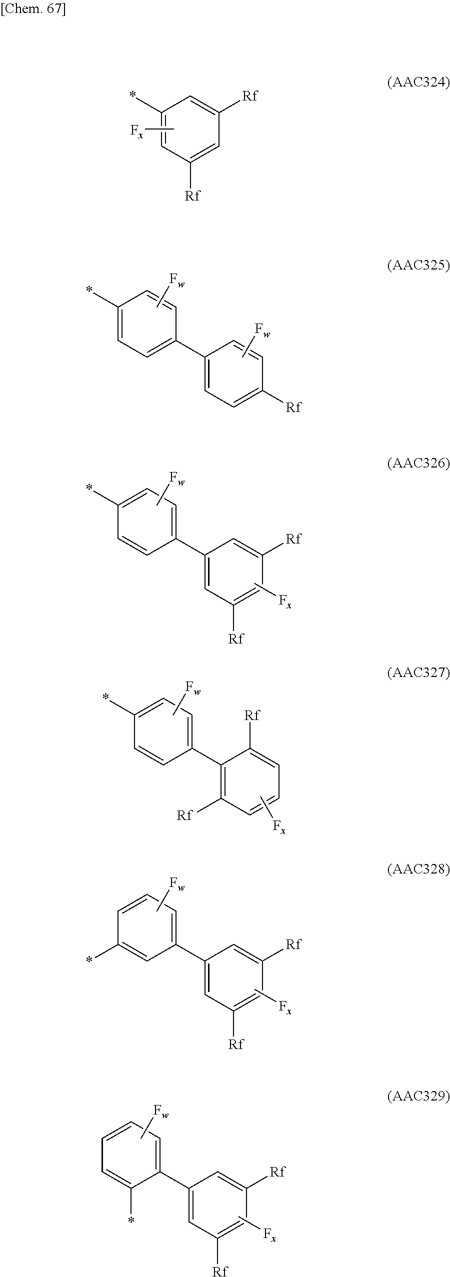

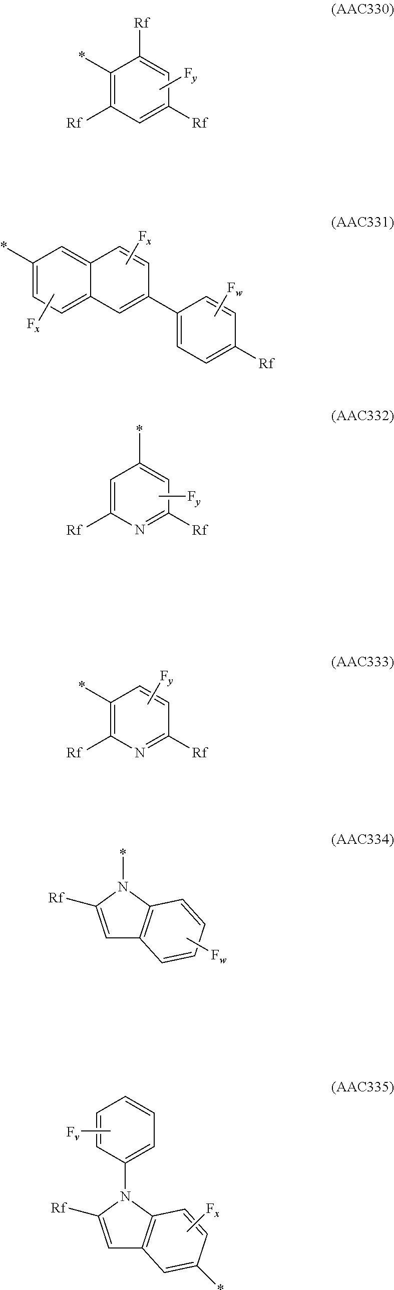

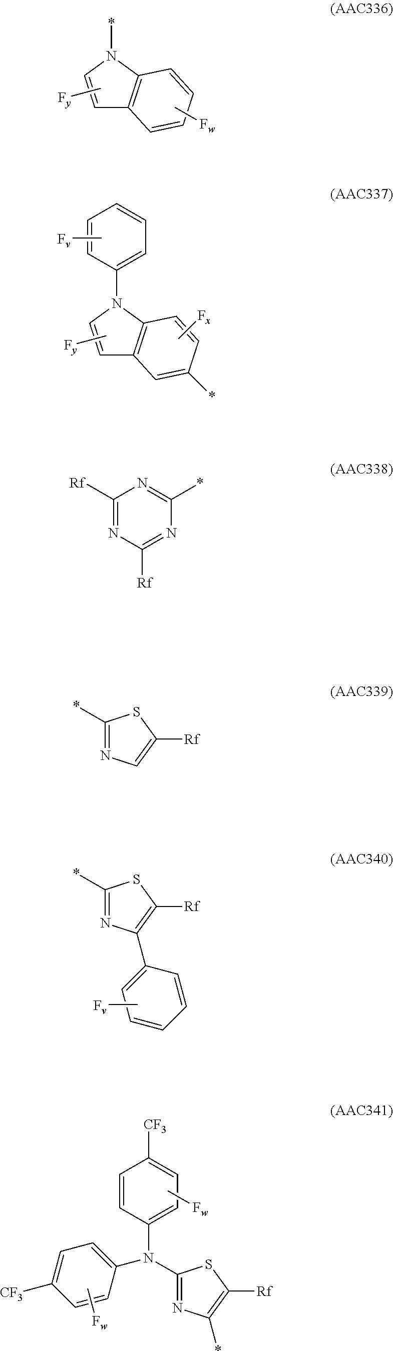

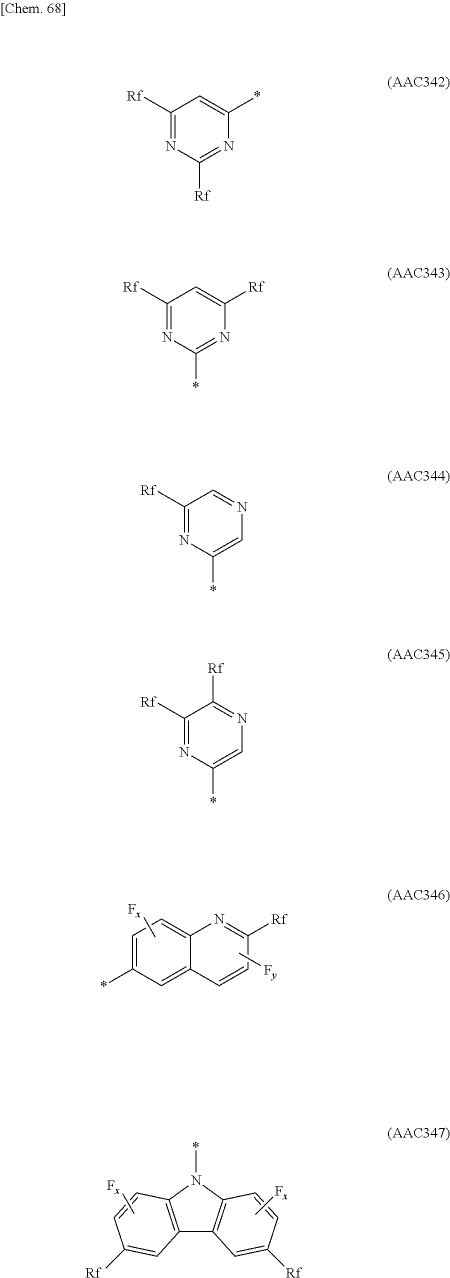

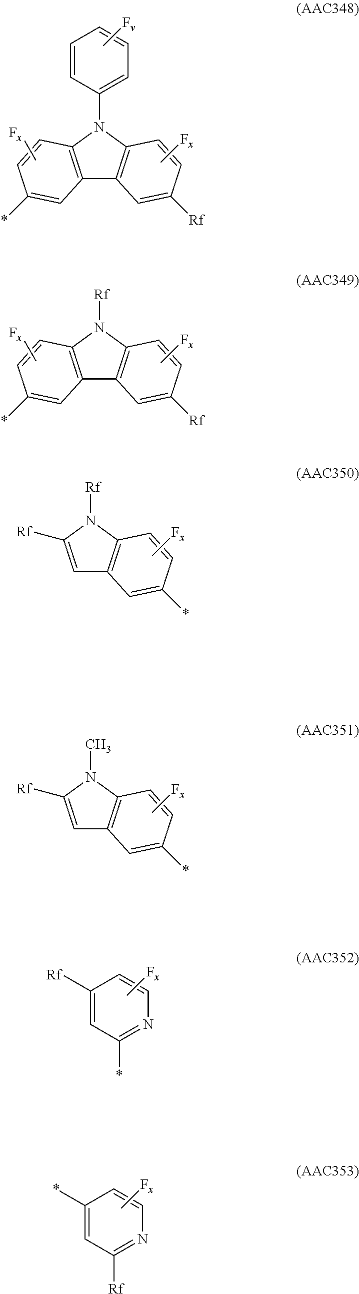

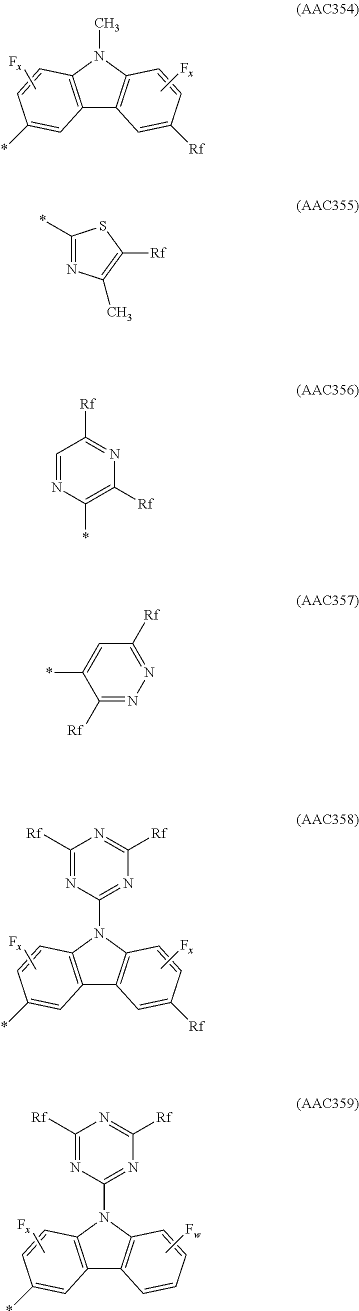

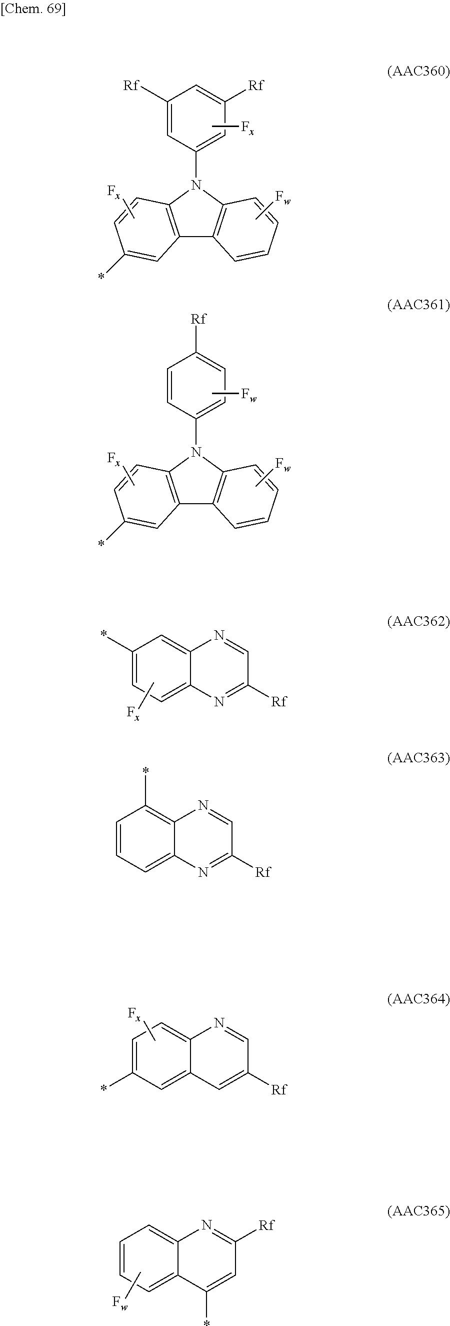

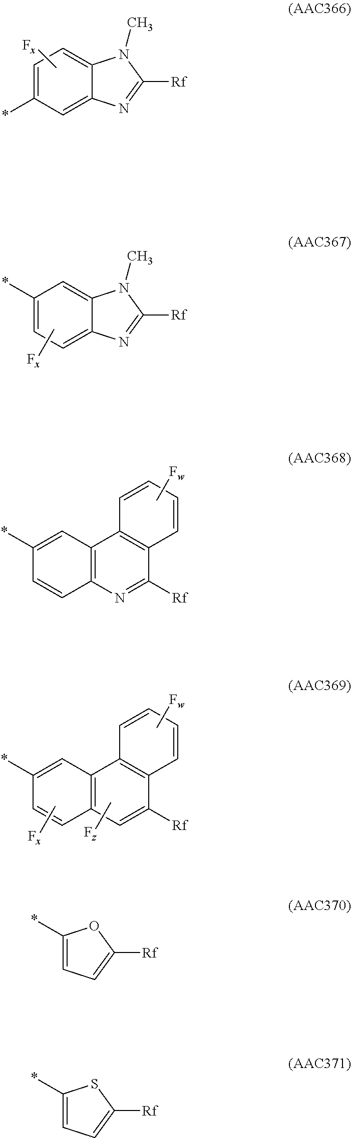

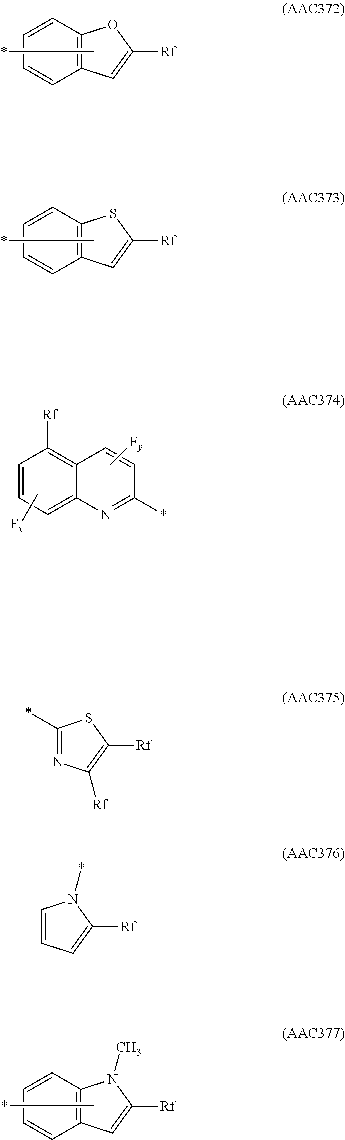

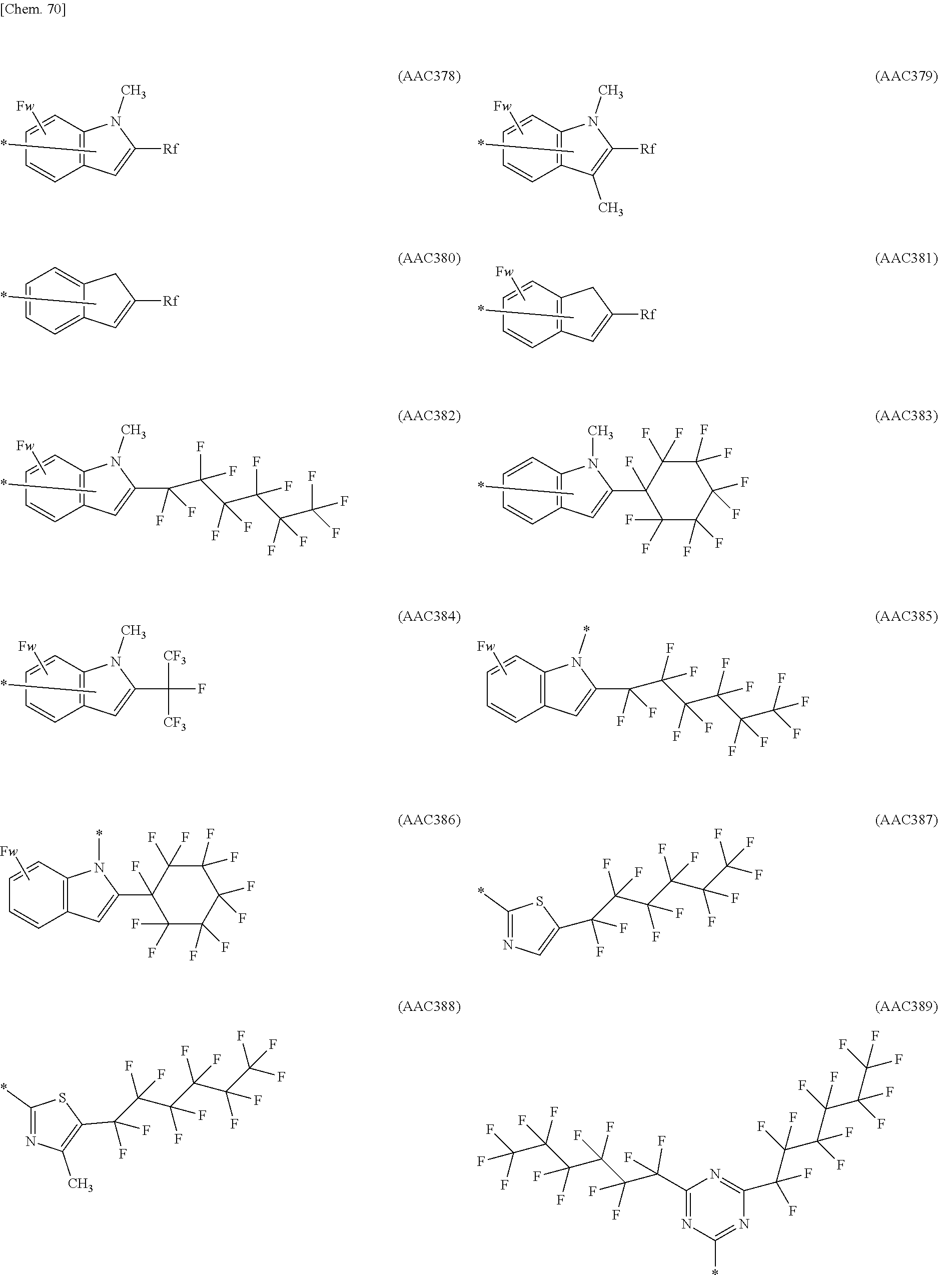

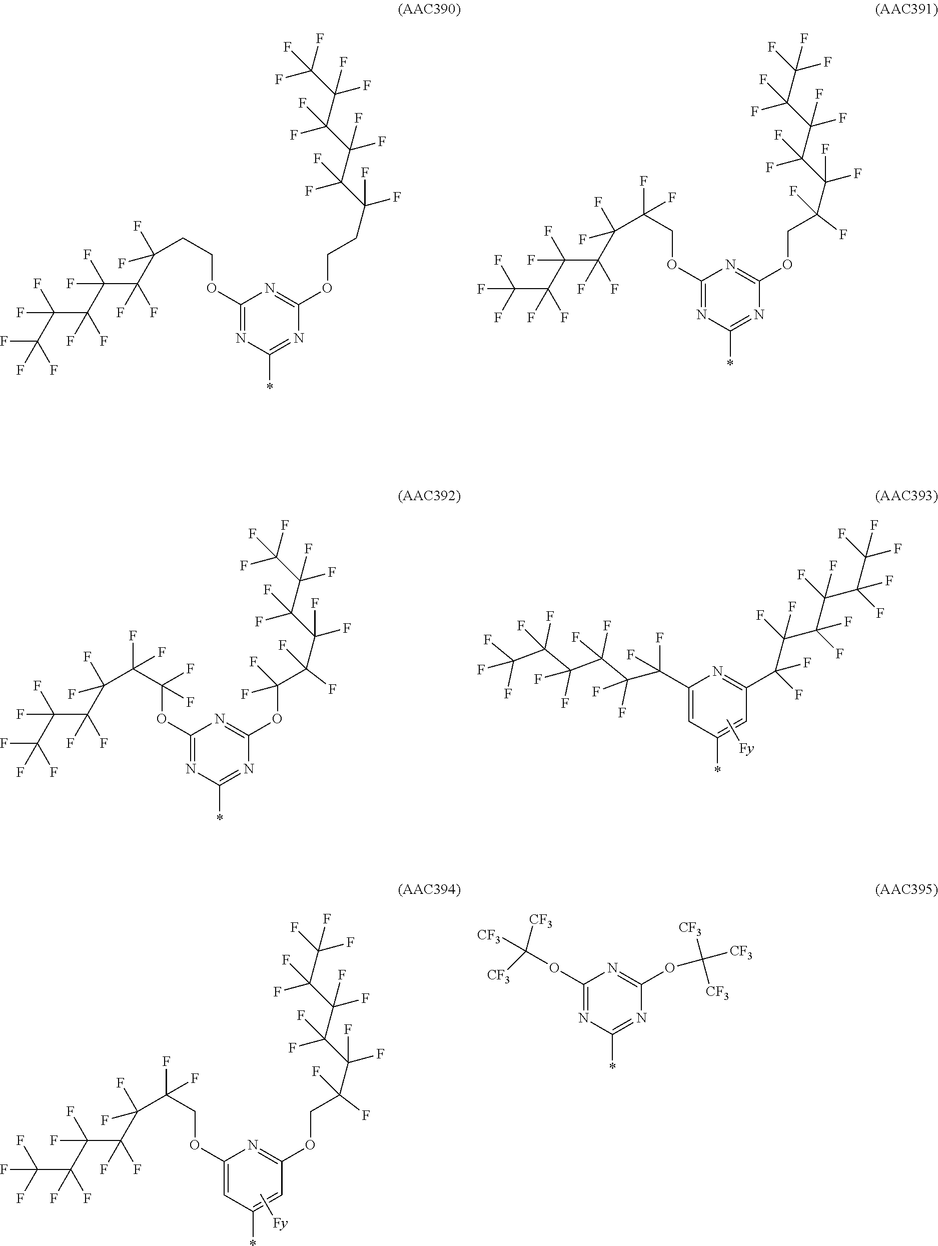

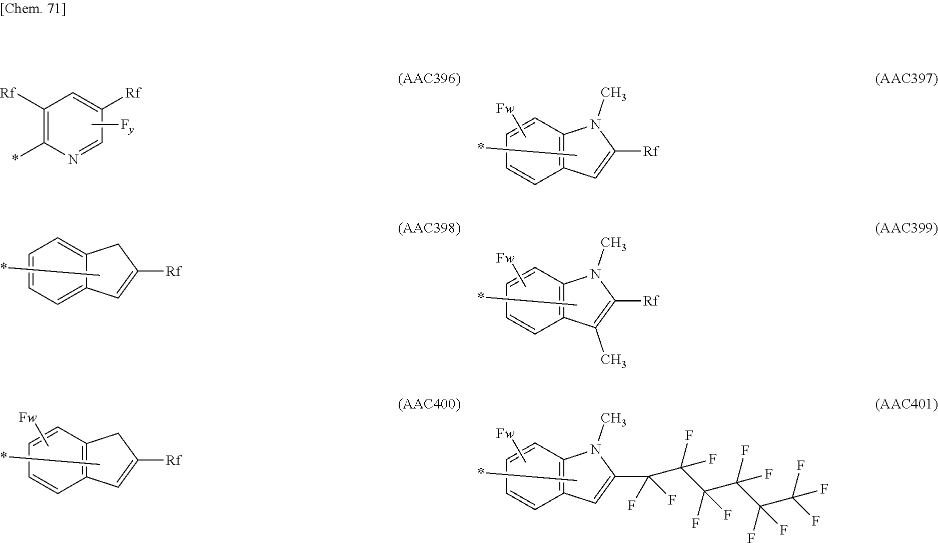

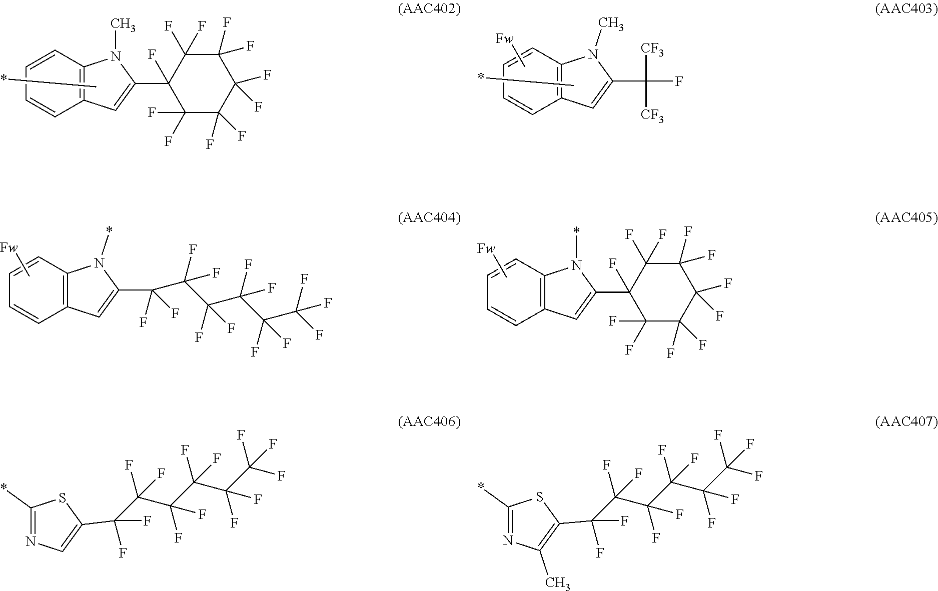

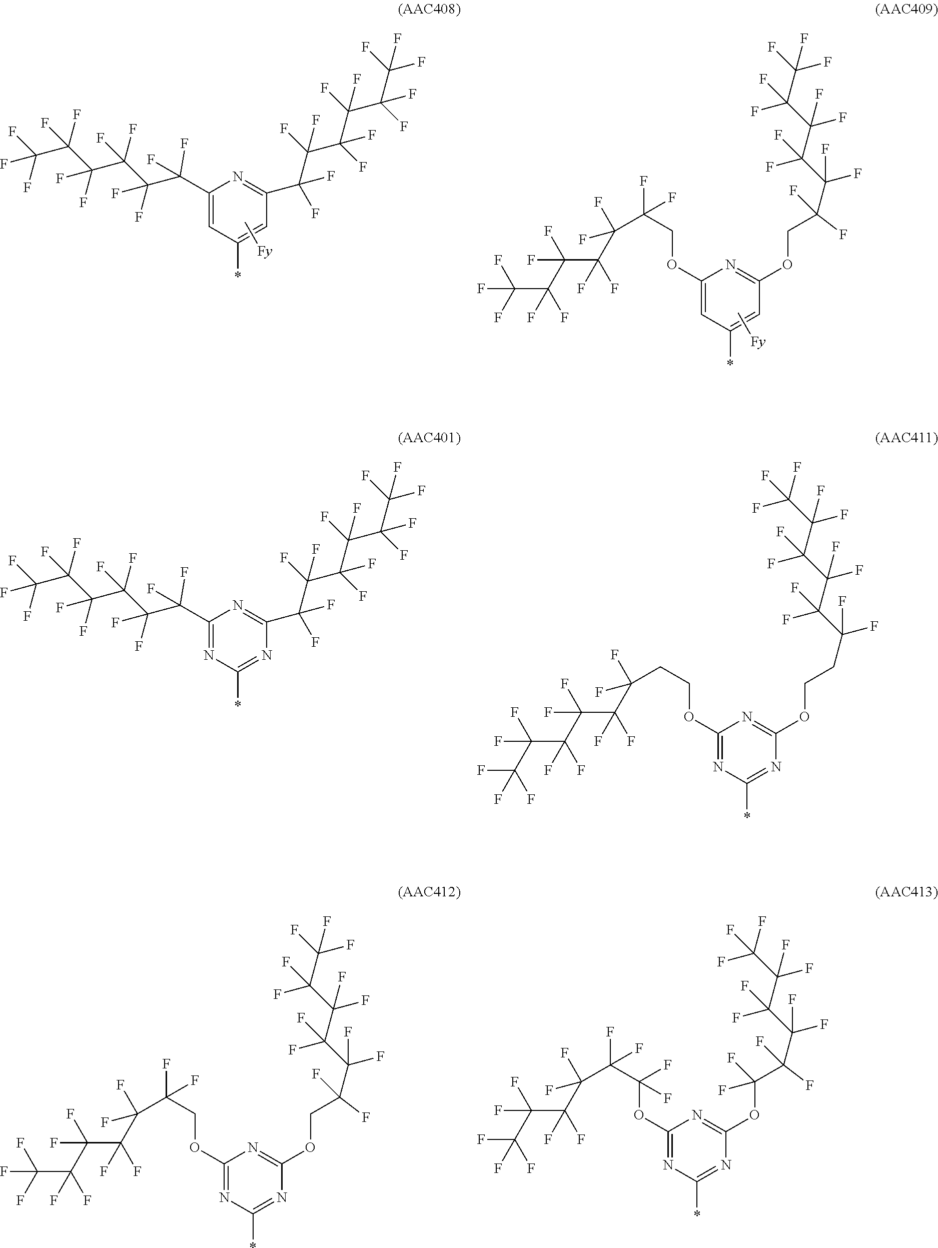

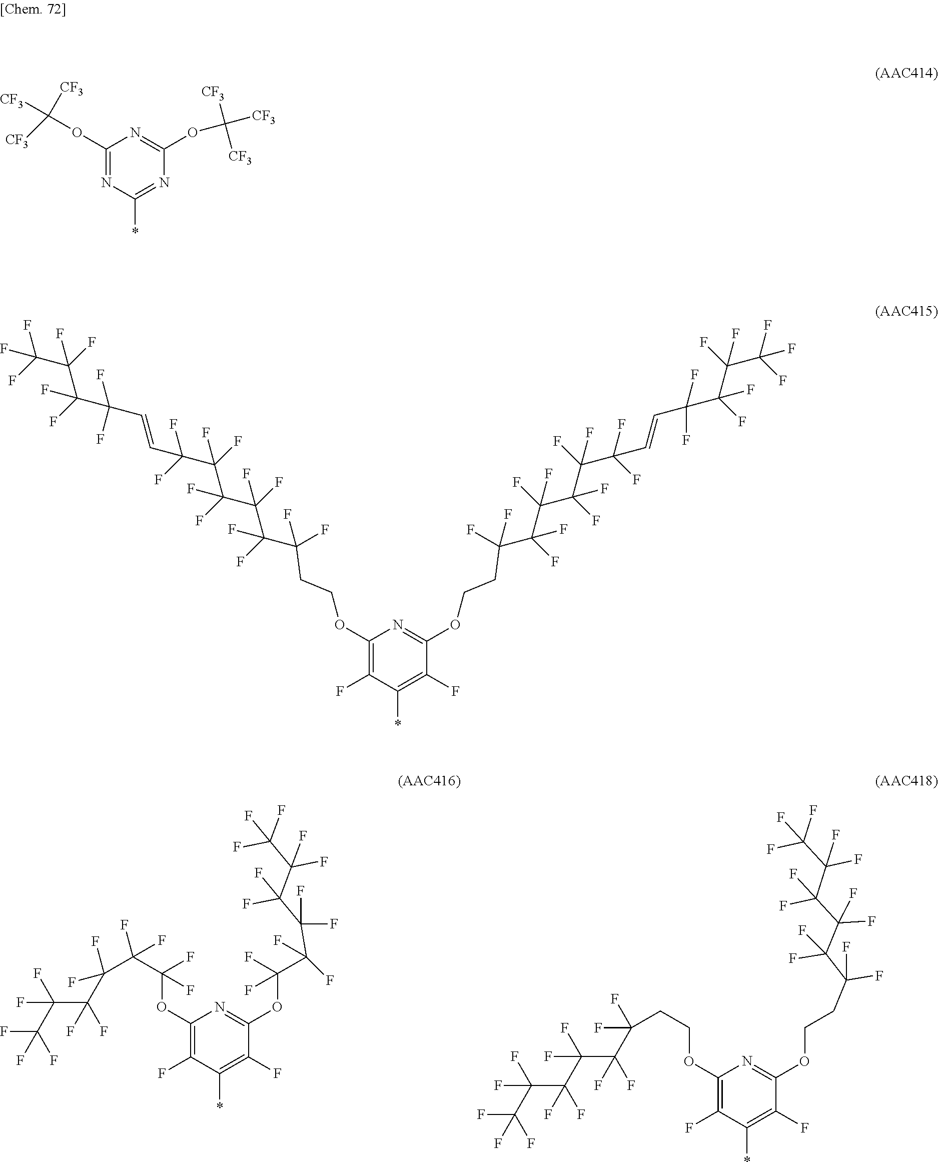 [SMILES: *c1nc(OC(C(F)(F)F)(C(F)(F)F)C(F)(F)F)nc(OC(C(F)(F)F)(C(F)(F)F)C(F)(F)F)n1 ; *c1c(F)c(OCCC(F)(F)C(F)(F)C(F)(F)C(F)(F)C(F)(F)C(F)(F)/C=C/C(F)(F)C(F)(F)C(F)(F)C(F)(F)F)nc(OCCC(F)(F)C(F)(F)C(F)(F)C(F)(F)C(F)(F)C(F)(F)/C=C/C(F)(F)C(F)(F)C(F)(F)C(F)(F)F)c1F ; *c1c(F)c(OC(F)(F)C(F)(F)C(F)(F)C(F)(F)C(F)(F)C(F)(F)F)nc(OC(F)(F)C(F)(F)C(F)(F)C(F)(F)C(F)(F)C(F)(F)F)c1F ; *c1c(F)c(OCCC(F)(F)C(F)(F)C(F)(F)C(F)(F)C(F)(F)C(F)(F)F)nc(OCCC(F)(F)C(F)(F)C(F)(F)C(F)(F)C(F)(F)C(F)(F)F)c1F]

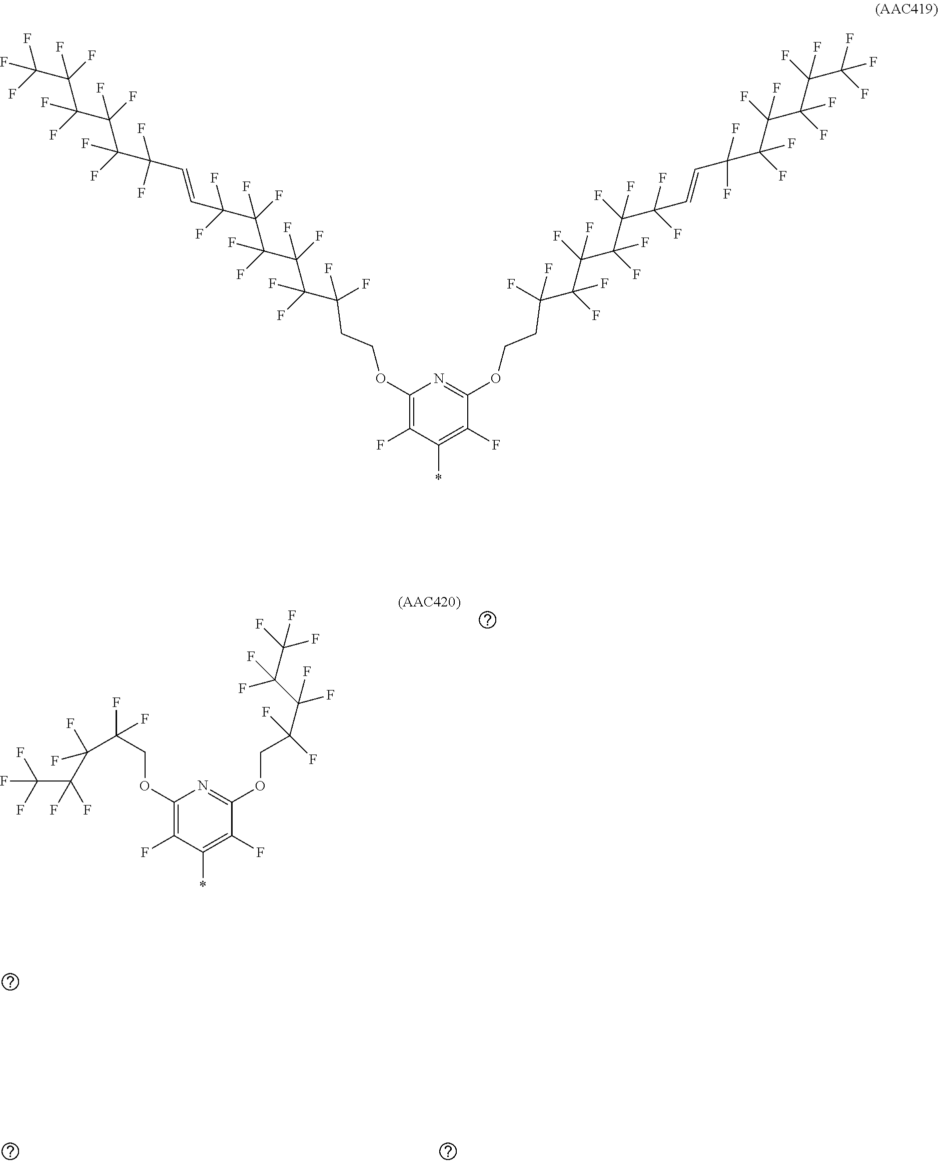 [SMILES: *c1c(F)c(OCCC(F)(F)C(F)(F)C(F)(F)C(F)(F)C(F)(F)C(F)(F)/C=C/C(F)(F)C(F)(F)C(F)(F)C(F)(F)C(F)(F)C(F)(F)F)nc(OCCC(F)(F)C(F)(F)C(F)(F)C(F)(F)C(F)(F)C(F)(F)/C=C/C(F)(F)C(F)(F)C(F)(F)C(F)(F)C(F)(F)C(F)(F)F)c1F ; *c1c(F)c(OCC(F)(F)C(F)(F)C(F)(F)C(F)(F)F)nc(OCC(F)(F)C(F)(F)C(F)(F)C(F)(F)F)c1F]

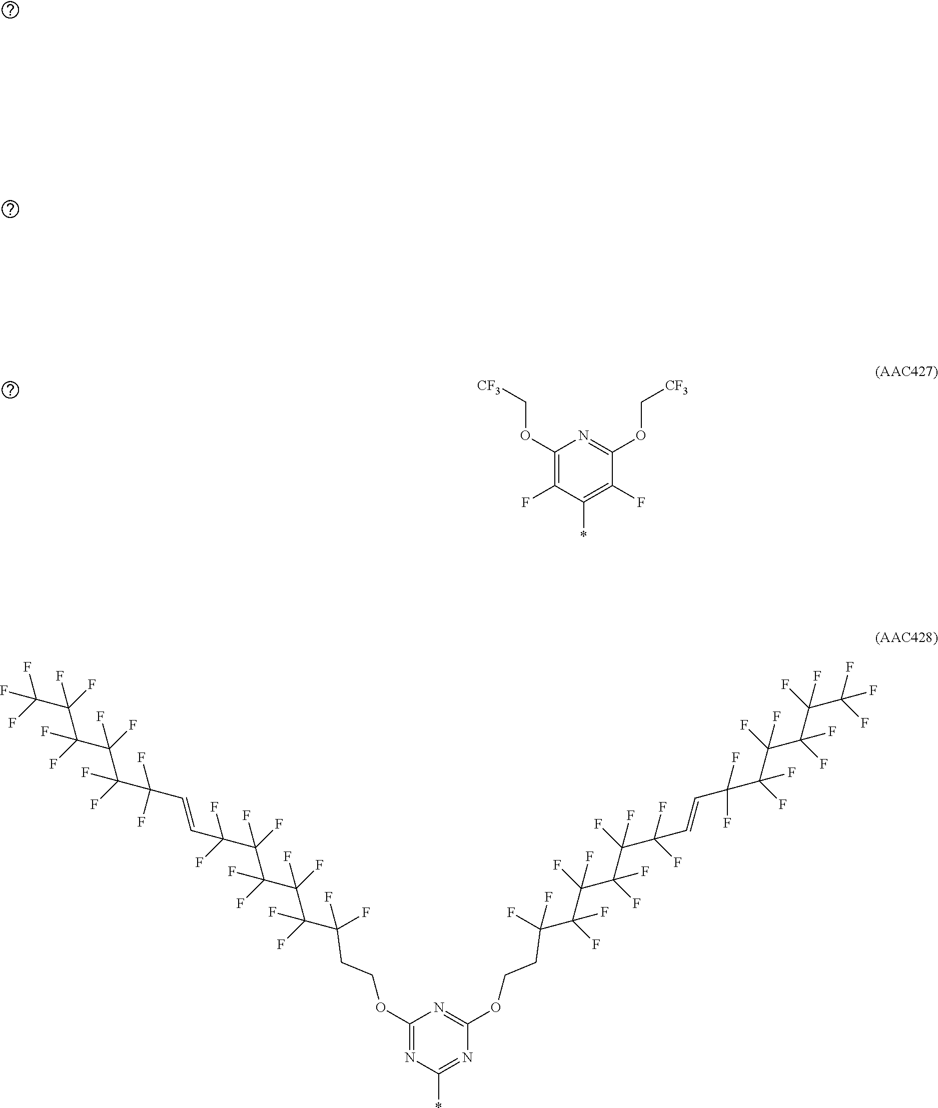 [SMILES: *c1c(F)c(OCC(F)(F)F)nc(OCC(F)(F)F)c1F ; *c1nc(OCCC(F)(F)C(F)(F)C(F)(F)C(F)(F)C(F)(F)C(F)(F)/C=C/C(F)(F)C(F)(F)C(F)(F)C(F)(F)C(F)(F)C(F)(F)F)nc(OCCC(F)(F)C(F)(F)C(F)(F)C(F)(F)C(F)(F)C(F)(F)/C=C/C(F)(F)C(F)(F)C(F)(F)C(F)(F)C(F)(F)C(F)(F)F)n1]

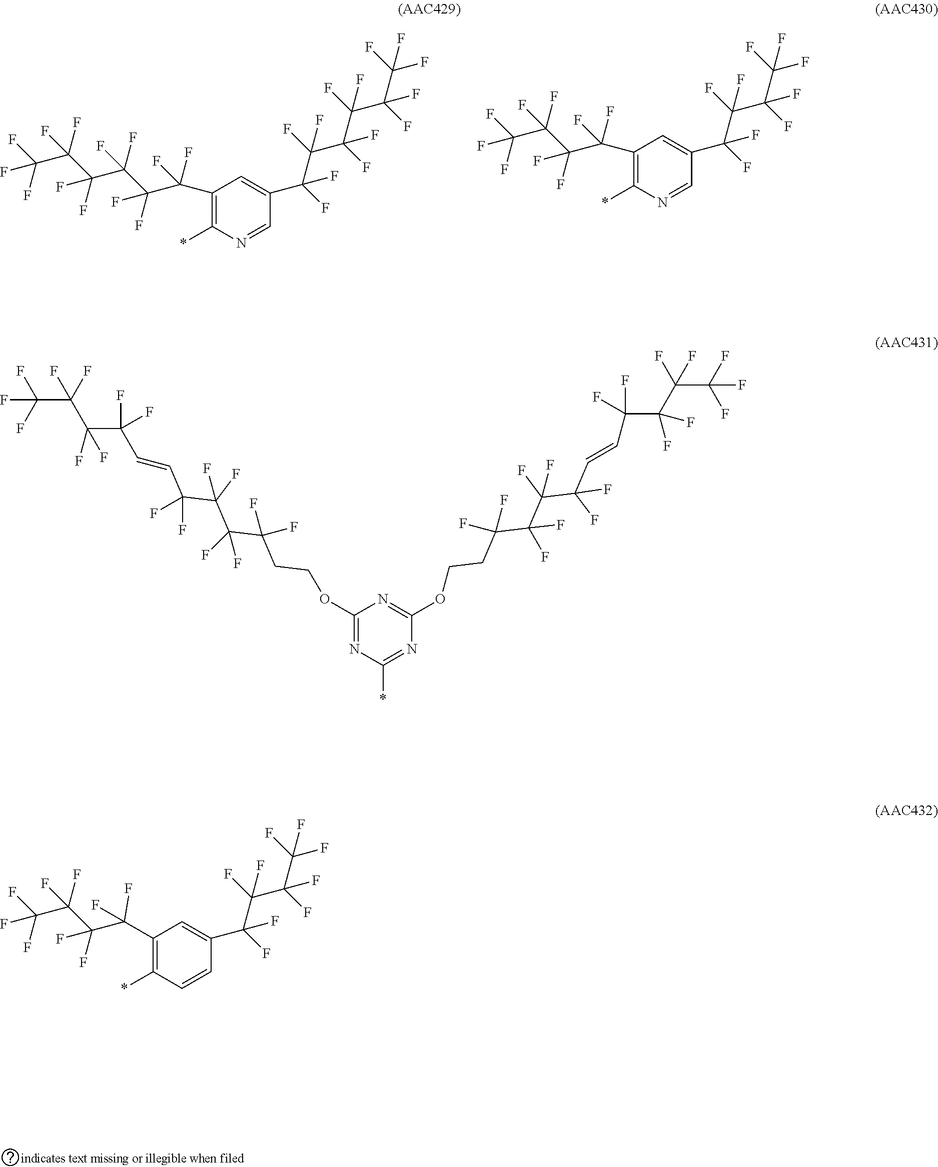 [SMILES: *c1ncc(C(F)(F)C(F)(F)C(F)(F)C(F)(F)C(F)(F)C(F)(F)F)cc1C(F)(F)C(F)(F)C(F)(F)C(F)(F)C(F)(F)C(F)(F)F ; *c1ncc(C(F)(F)C(F)(F)C(F)(F)C(F)(F)F)cc1C(F)(F)C(F)(F)C(F)(F)C(F)(F)F ; *c1nc(OCCC(F)(F)C(F)(F)C(F)(F)C(F)(F)/C=C/C(F)(F)C(F)(F)C(F)(F)C(F)(F)F)nc(OCCC(F)(F)C(F)(F)C(F)(F)C(F)(F)/C=C/C(F)(F)C(F)(F)C(F)(F)C(F)(F)F)n1 ; *c1ccc(C(F)(F)C(F)(F)C(F)(F)C(F)(F)F)cc1C(F)(F)C(F)(F)C(F)(F)C(F)(F)F]

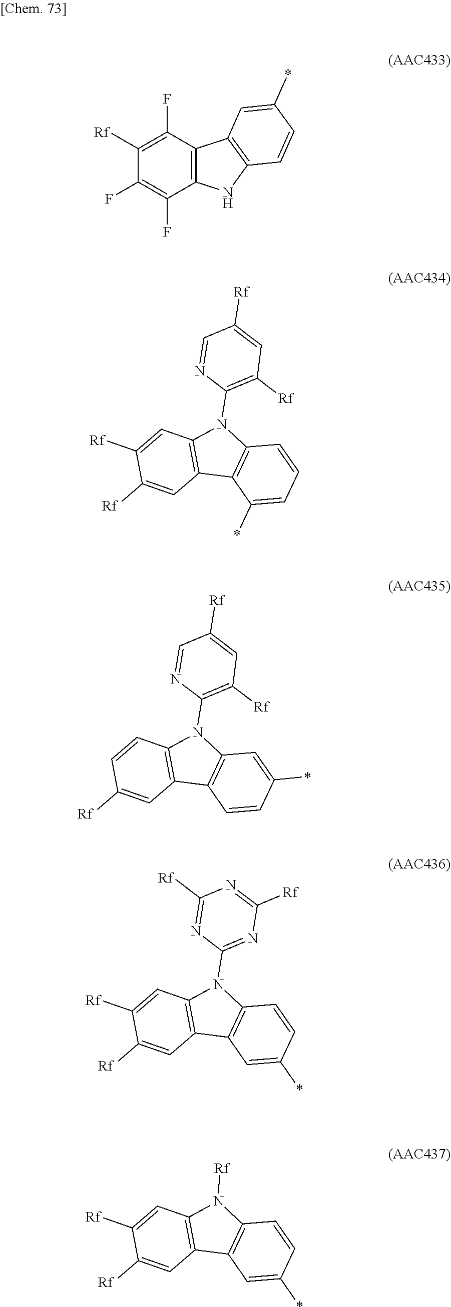

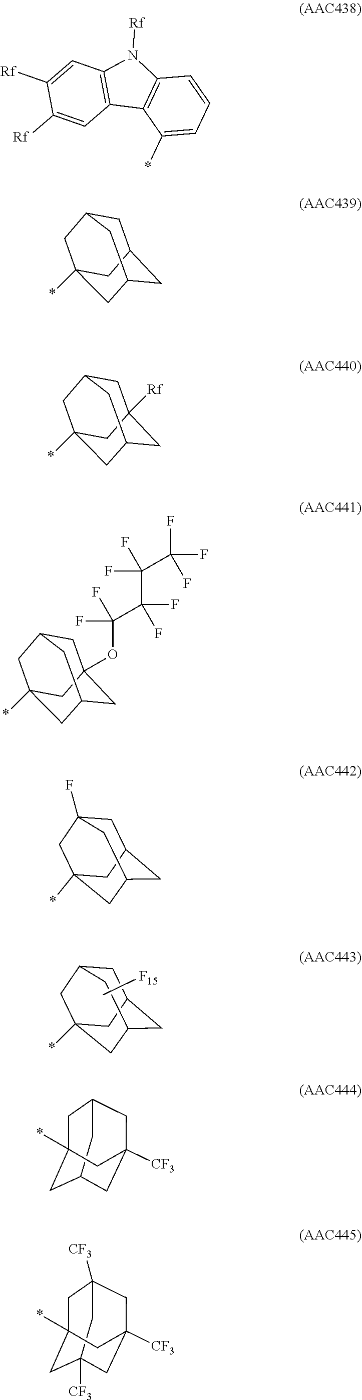

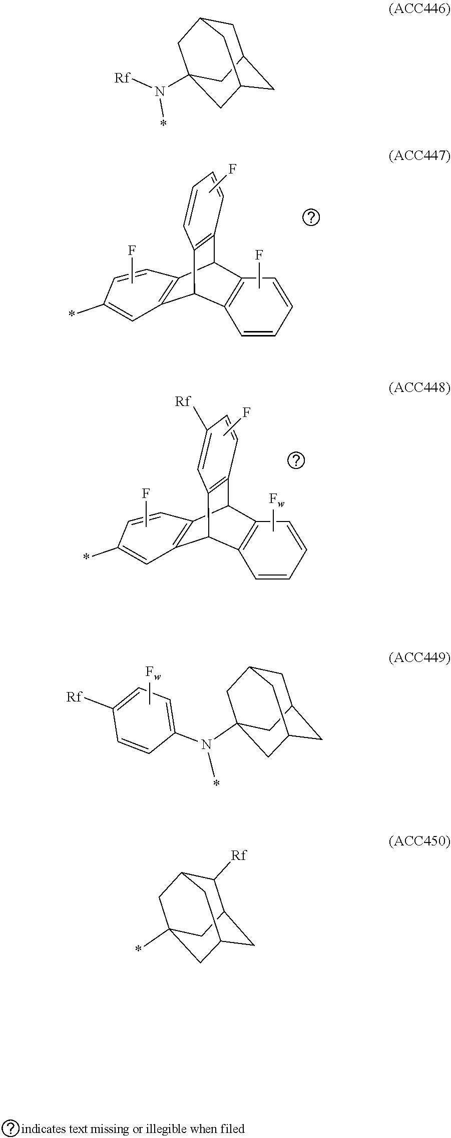

### [Formula (121)]

R¹⁰¹ to R¹⁰⁵ are represented by the formula (121):

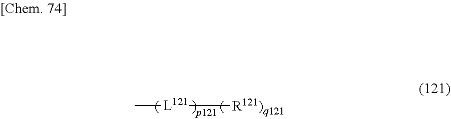

L¹²¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR¹¹¹.

R¹¹¹ and R¹²¹ each independently denote
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 1 to 18 carbon atoms, or
H, D (deuterium atom) or F (fluorine atom).

p121 each independently denotes an integer in the range of 0 to 12.

q121 each independently denotes an integer in the range of 1 to 6.

p121 each independently denotes an integer in the range of 0 to 12. p121 preferably denotes an integer in the range of 1 to 12, more preferably an integer in the range of 1 to 11, still more preferably an integer in the range of 1 to 10, still more preferably an integer in the range of 2 to 10.

q121 each independently denotes an integer in the range of 1 to 6. p121 preferably denotes an integer in the range of 2 to 6, more preferably an integer in the range of 3 to 6, still more preferably an integer in the range of 3 to 5.

R¹⁰¹ to R¹⁰⁵ are not particularly limited and may have structures, for example, represented by the following (AB1) to (AB31).

The mark “*” in the following structures denotes a binding site.

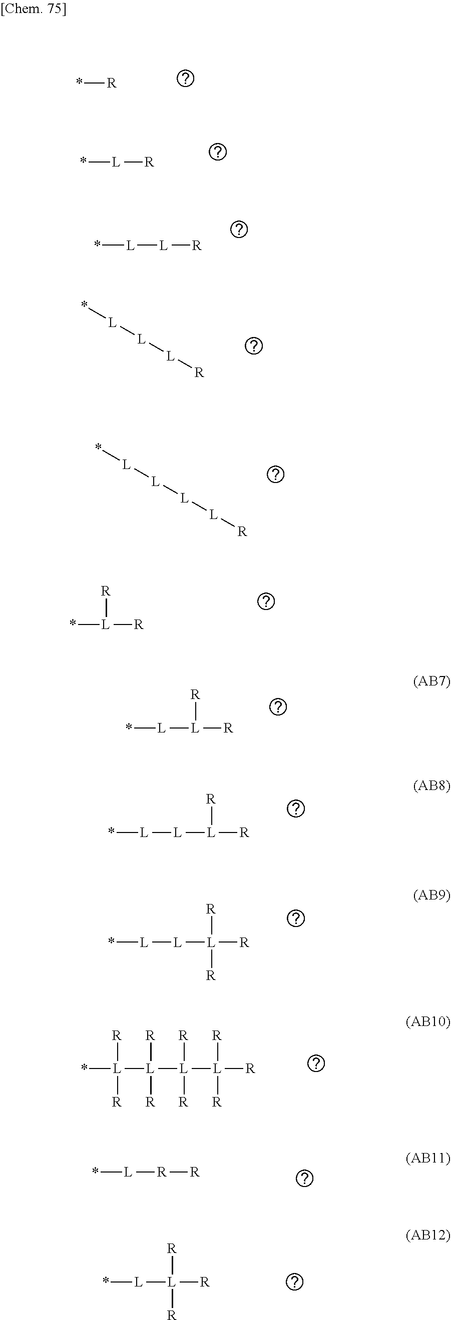

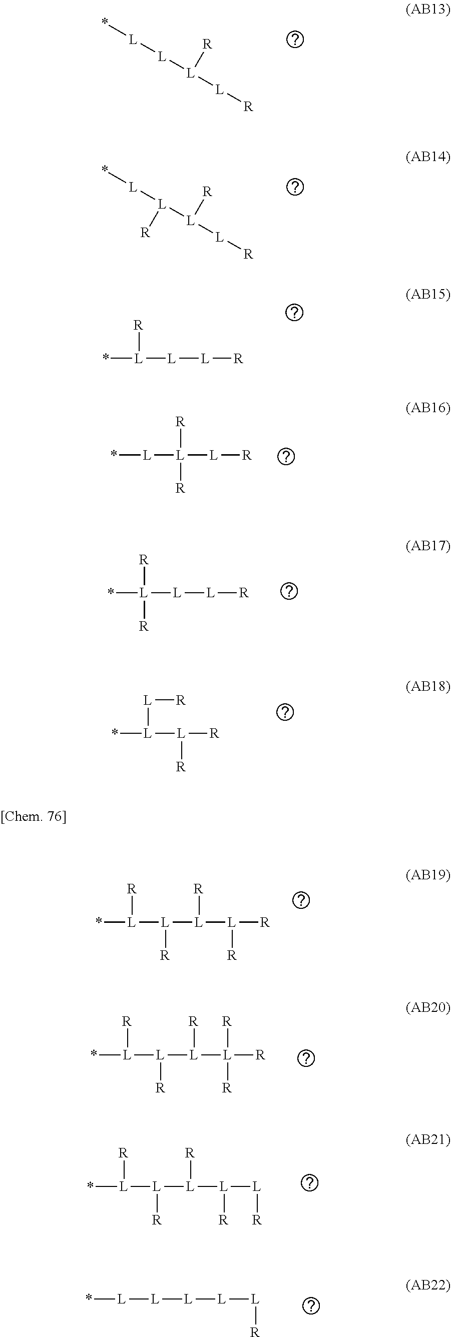

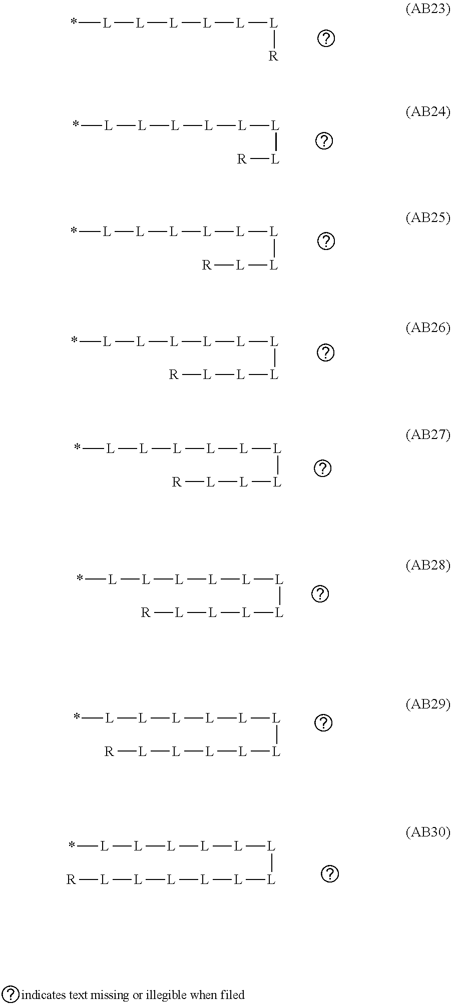

### Preferred Embodiments of R¹⁰¹, R¹⁰², R¹⁰³, R¹⁰⁴ and R¹⁰⁵

R¹⁰¹, R¹⁰², R¹⁰³, R¹⁰⁴ and R¹⁰⁵ preferably each independently denote a structure represented by the formula (131):

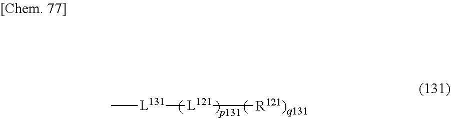

L¹²¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O, S or NR¹¹¹.

L¹³¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O or S.

R¹¹¹ and R¹²¹ each independently denote
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 1 to 18 carbon atoms, or
H, D (deuterium atom) or F (fluorine atom).

p131 each independently denotes an integer in the range of 0 to 11.

q131 each independently denotes an integer in the range of 1 to 6.

### [L¹²¹]

L¹²¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O, S or NR¹¹¹.

Preferably, the monocyclic, linked or condensed aromatic hydrocarbon group represented by L¹²¹ is a phenyl group or has a structure in which a plurality of benzene rings are linked or condensed.

Preferably, the monocyclic, linked or condensed heteroaromatic group represented by L¹²¹ has N, O or S as a heteroatom and is a 5-membered ring, a 6-membered ring or a structure in which these or a plurality of benzene rings are condensed.

Preferably, the cyclic heteroaliphatic hydrocarbon group represented by L¹²¹ has N, O or S as a heteroatom and is a 5-membered ring, a 6-membered ring or a condensed structure thereof.

More preferably, a compound that provides the monocyclic, linked or condensed divalent to tetravalent aromatic hydrocarbon group represented by L¹²¹ is benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene,
a compound that provides the monocyclic, linked or condensed divalent to tetravalent heteroaromatic group is pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene,
a compound that provides the cyclic divalent to tetravalent aliphatic hydrocarbon group is adamantane, diamantane or cyclohexane, and
a compound that provides the cyclic divalent to tetravalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

Still more preferably, the monocyclic, linked or condensed divalent to tetravalent aromatic hydrocarbon group represented by L¹²¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene or triptycene,
the monocyclic, linked or condensed divalent to tetravalent heteroaromatic group is pyridine, pyrimidine, pyrazine, triazine, carbazole, furan, thiophene, benzofuran, benzothiophene, dibenzofuran, dibenzothiophene, thiazole, phenothiazine, dibenzo-1,4-dioxin or benzothiazole,
the cyclic divalent to tetravalent aliphatic hydrocarbon group is adamantane, diamantane or cyclohexane, and
a compound that provides the cyclic divalent to tetravalent heteroaliphatic hydrocarbon group is morpholine, piperazine, homopiperazine, hexahydro-1,3,5-triazine, 1,4-dioxin, 1,4-dithiane or 1,4,7,10-tetraazacyclododecane.

Still more preferably, the monocyclic, linked or condensed divalent to tetravalent aromatic hydrocarbon group represented by L¹²¹ is phenyl, biphenyl, naphthalene, fluorene, spirobifluorene, phenanthrene or triptycene,
the monocyclic, linked or condensed divalent to tetravalent heteroaromatic group is pyridine, pyrimidine, pyrazine, triazine, carbazole, furan, thiophene, benzofuran, benzothiophene, dibenzofuran, dibenzothiophene, thiazole, phenothiazine, dibenzo-1,4-dioxin or benzothiazole,
the cyclic divalent to tetravalent aliphatic hydrocarbon group is adamantane, diamantane or cyclohexane, and
a compound that provides the cyclic divalent to tetravalent heteroaliphatic hydrocarbon group is morpholine, piperazine, homopiperazine, hexahydro-1,3,5-triazine, 1,4-dioxin, 1,4-dithiane or 1,4,7,10-tetraazacyclododecane.

L¹²¹ preferably each independently denotes
a monocyclic, linked or condensed divalent to tetravalent aromatic hydrocarbon group,
a monocyclic, linked or condensed divalent to tetravalent heteroaromatic group,
a linear, branched or cyclic divalent to tetravalent aliphatic hydrocarbon group,
a cyclic divalent to tetravalent heteroaliphatic hydrocarbon group, or
O or S.

When L¹²¹ denotes a substituted aromatic hydrocarbon group, a substituted heteroaromatic group, a substituted aliphatic hydrocarbon group or a substituted heteroaliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a cyano group, a fluorine atom, a deuterium atom and a structure represented by the formula (001), and
a combination of these groups.

A substituent of L¹²¹ is preferably each independently substituted with one or more groups selected from the group consisting of
a methyl group, a trifluoromethyl group, a trifluoromethoxy group, an alkyl group with 2 to 10 carbon atoms, a cyano group, a deuterium atom, a fluorine atom, a phenyl group, a biphenylyl group, a naphthyl group, a phenanthryl group, a pyridyl group, a pyrimidyl group, a pyrazyl group, a triazyl group, an indolyl group, a carbazolyl group, a benzothienyl group, a dibenzothienyl group, a benzofuranyl group, a dibenzofuranyl group, a thiazolyl group, a benzothiazolyl group and a structure represented by the formula (001), and
a combination of these groups.

A substituent of L¹²¹ is preferably each independently substituted with one or more groups selected from the group consisting of
a methyl group, a trifluoromethyl group, a trifluoromethoxy group, an alkyl group with 2 to 10 carbon atoms, a fluorine atom, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group, a thiazolyl group and a structure represented by the formula (001), and
a combination of these groups.

A substituent of L¹²¹ is more preferably each independently
(iv) a methyl group, a methoxy group, an alkyl group with 2 to 10 carbon atoms, an alkoxy group with 2 to 10 carbon atoms, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group or a thiazolyl group,
(v) the group represented by (iv) substituted with one or more groups selected from the group consisting of a fluorine atom, a perfluoroalkyl group with 1 to 10 carbon atoms and a perfluoroalkoxy group with 1 to 10 carbon atoms, or
(vi) a fluorine atom or a structure represented by the formula (001).

A substituent of L¹²¹ is still more preferably each independently
(iv′) a methyl group, an ethyl group, a butyl group, a methoxy group, an ethoxy group, a butoxy group, a phenyl group, a naphthyl group, a pyridyl group, a pyrimidyl group, a pyrazyl group, a triazyl group, a carbazolyl group, a dibenzothienyl group, a dibenzofuranyl group or a thiazolyl group,
(v′) the group represented by (iv′) substituted with one or more groups selected from the group consisting of a fluorine atom, a perfluoroalkyl group with 1 to 6 carbon atoms and a perfluoroalkoxy group with 1 to 6 carbon atoms, or
(vi′) a fluorine atom or a structure represented by the formula (001).

A substituent of L¹²¹ is still more preferably each independently a trifluoromethyl group, a trifluoromethoxy group, an alkyl group having 2 to 10 carbon atoms and substituted with 3 or more fluorine atoms, an alkoxy group having 2 to 10 carbon atoms and substituted with 3 or more fluorine atoms or a fluorine atom.

### Specific Example of L¹²¹

L¹²¹ may have a structure, for example, represented above by one of (AAB1) to (AAB803).

### [L¹³¹]

L¹³¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O or S.

Preferably, the monocyclic, linked or condensed aromatic hydrocarbon group represented by L¹³¹ is a phenyl group or has a structure in which a plurality of benzene rings are linked or condensed.

Preferably, the monocyclic, linked or condensed heteroaromatic group represented by L¹³¹ has N, O or S as a heteroatom and is a 5-membered ring, a 6-membered ring or a structure in which these or a plurality of benzene rings are condensed.

Preferably, the cyclic heteroaliphatic hydrocarbon group represented by L¹³¹ has N, O or S as a heteroatom and is a 5-membered ring, a 6-membered ring or a condensed structure thereof.

More preferably, a compound that provides the monocyclic, linked or condensed divalent to tetravalent aromatic hydrocarbon group represented by L¹³¹ is benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene,
a compound that provides the monocyclic, linked or condensed divalent to tetravalent heteroaromatic group is pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene,
a compound that provides the cyclic divalent to tetravalent aliphatic hydrocarbon group is adamantane, diamantane or cyclohexane, and
a compound that provides the cyclic divalent to tetravalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

Still more preferably, the monocyclic, linked or condensed aromatic hydrocarbon group represented by L¹³¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene or corannulene,
the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole or benzothiazole,
the linear, branched or cyclic aliphatic hydrocarbon group is adamantane, diamantane or cyclohexane, and
a compound that provides the cyclic divalent to tetravalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane or 1,4,7,10-tetraazacyclododecane.

Preferably, when L¹³¹ denotes a substituted aromatic hydrocarbon group, a substituted heteroaromatic group, a substituted aliphatic hydrocarbon group or a cyclic heteroaliphatic hydrocarbon group, these groups are each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a cyano group, a fluorine atom, a deuterium atom and a structure represented by the formula (001), and
a combination of these groups.

A substituent of L¹³¹ is preferably each independently substituted with one or more groups selected from the group consisting of
a methyl group, a trifluoromethyl group, a trifluoromethoxy group, an alkyl group with 2 to 10 carbon atoms, a cyano group, a deuterium atom, a fluorine atom, a phenyl group, a biphenylyl group, a naphthyl group, a phenanthryl group, a pyridyl group, a pyrimidyl group, a pyrazyl group, a triazyl group, an indolyl group, a carbazolyl group, a benzothienyl group, a dibenzothienyl group, a benzofuranyl group, a dibenzofuranyl group, a thiazolyl group, a benzothiazolyl group and a structure represented by the formula (001), and
a combination of these groups.

More preferably, a substituent of L¹³¹ is each independently substituted with one or more groups selected from the group consisting of
a methyl group, a trifluoromethyl group, a trifluoromethoxy group, an alkyl group with 2 to 10 carbon atoms, a fluorine atom, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group, a thiazolyl group and a structure represented by the formula (001), and
a combination of these groups.

A substituent of L¹³¹ is still more preferably each independently
(iv) a methyl group, a methoxy group, an alkyl group with 2 to 10 carbon atoms, an alkoxy group with 2 to 10 carbon atoms, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group or a thiazolyl group,
(v) the group represented by (iv) substituted with one or more groups selected from the group consisting of a fluorine atom, a perfluoroalkyl group with 1 to 10 carbon atoms and a perfluoroalkoxy group with 1 to 10 carbon atoms, or
(vi) a fluorine atom or a structure represented by the formula (001).

A substituent of L¹³¹ is still more preferably each independently
(iv′) a methyl group, an ethyl group, a butyl group, a methoxy group, an ethoxy group, a butoxy group, a phenyl group, a naphthyl group, a pyridyl group, a pyrimidyl group, a pyrazyl group, a triazyl group, a carbazolyl group, a dibenzothienyl group, a dibenzofuranyl group or a thiazolyl group,
(v′) the group represented by (iv′) substituted with one or more groups selected from the group consisting of a fluorine atom, a perfluoroalkyl group with 1 to 6 carbon atoms and a perfluoroalkoxy group with 1 to 6 carbon atoms, or
(vi′) a fluorine atom or a structure represented by the formula (001).

A substituent of L¹³¹ is still more preferably each independently a trifluoromethyl group, a trifluoromethoxy group, an alkyl group having 2 to 10 carbon atoms and substituted with 3 or more fluorine atoms, an alkoxy group having 2 to 10 carbon atoms and substituted with 3 or more fluorine atoms, a fluorine atom or a structure represented by the formula (001).

### Specific Example of L¹³¹

L¹³¹ may have a structure, for example, represented above by one of (AAB1) to (AAB802).

### [R¹²¹]

R¹²¹ each independently denotes
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 3 to 18 carbon atoms, or
H, D (deuterium atom) or F (fluorine atom).

Preferably, the monocyclic, linked or condensed aromatic hydrocarbon group represented by R¹²¹ is a phenyl group or has a structure in which a plurality of benzene rings are linked or condensed.

Preferably, the monocyclic, linked or condensed heteroaromatic group represented by R¹²¹ has N, O or S as a heteroatom and is a 5-membered ring, a 6-membered ring or a structure in which these or a plurality of benzene rings are condensed.

Preferably, the cyclic heteroaliphatic hydrocarbon group represented by R¹²¹ has N, O or S as a heteroatom and is a 5-membered ring, a 6-membered ring or a condensed structure thereof.

More preferably, a compound that provides the monocyclic, linked or condensed monovalent aromatic hydrocarbon group represented by R¹²¹ is benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene,
a compound that provides the monocyclic, linked or condensed monovalent heteroaromatic group is pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene,
a compound that provides the linear, branched or cyclic monovalent aliphatic hydrocarbon group is adamantane, diamantane, cyclohexane or methane, and
a compound that provides the cyclic monovalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

Still more preferably, the monocyclic, linked or condensed aromatic hydrocarbon group represented by R¹²¹ is phenyl, biphenyl, terphenyl, naphthalene, fluorene, spirobifluorene, 9,9-dimethylfluorene, 9,9-diphenylfluorene, benzofluorene, phenanthrene, fluoranthene or triptycene,
the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole or benzothiazole,
the linear, branched or cyclic aliphatic hydrocarbon group is adamantane, diamantane, cyclohexane or methane, and
a compound that provides the cyclic heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

Still more preferably, the monocyclic, linked or condensed aromatic hydrocarbon group represented by R¹²¹ is phenyl, biphenyl, terphenyl, naphthalene, fluorene, spirobifluorene, 9,9-dimethylfluorene, 9,9-diphenylfluorene, benzofluorene, phenanthrene, fluoranthene or triptycene,
the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole or benzothiazole,
the linear, branched or cyclic aliphatic hydrocarbon group is adamantane, diamantane or cyclohexane, and
a compound that provides the cyclic heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane or 1,4,7,10-tetraazacyclododecane.

When R¹²¹ denotes a substituted aromatic hydrocarbon group, a substituted heteroaromatic group, a substituted aliphatic hydrocarbon group or a substituted heteroaliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a cyano group, a fluorine atom, a deuterium atom and a structure represented by the formula (001), and
a combination of these groups.

A substituent of R¹²¹ is preferably each independently substituted with one or more groups selected from the group consisting of
a methyl group, a trifluoromethyl group, a trifluoromethoxy group, an alkyl group with 2 to 10 carbon atoms, a cyano group, a deuterium atom, a fluorine atom, a phenyl group, a biphenylyl group, a naphthyl group, a phenanthryl group, a pyridyl group, a pyrimidyl group, a pyrazyl group, a triazyl group, an indolyl group, a carbazolyl group, a benzothienyl group, a dibenzothienyl group, a benzofuranyl group, a dibenzofuranyl group, a thiazolyl group, a benzothiazolyl group and a structure represented by the formula (001), and
a combination of these groups.

A substituent of R¹²¹ is more preferably each independently substituted with one or more groups selected from the group consisting of
a methyl group, a trifluoromethyl group, a trifluoromethoxy group, an alkyl group with 2 to 10 carbon atoms, a fluorine atom, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group, a thiazolyl group and a structure represented by the formula (001), and
a combination of these groups.

A substituent of R¹²¹ is still more preferably each independently
(vii) a methyl group, a methoxy group, an alkyl group with 2 to 10 carbon atoms, an alkoxy group with 2 to 10 carbon atoms, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group or a thiazolyl group,
(viii) the group represented by (vii) substituted with one or more groups selected from the group consisting of a fluorine atom, a perfluoroalkyl group with 1 to 10 carbon atoms and a perfluoroalkoxy group with 1 to 10 carbon atoms, or
(ix) a fluorine atom or a structure represented by the formula (001).

A substituent of R¹²¹ is still more preferably each independently
(vii′) a methyl group, an ethyl group, a butyl group, a methoxy group, an ethoxy group, a butoxy group, a phenyl group, a naphthyl group, a pyridyl group, a pyrimidyl group, a pyrazyl group, a triazyl group, a carbazolyl group, a dibenzothienyl group, a dibenzofuranyl group or a thiazolyl group,
(viii′) the group represented by (vii′) substituted with one or more groups selected from the group consisting of a fluorine atom, a perfluoroalkyl group with 1 to 6 carbon atoms and a perfluoroalkoxy group with 1 to 6 carbon atoms, or
(ix′) a fluorine atom or a structure represented by the formula (001).

A substituent of R¹²¹ preferably each independently denotes
a trifluoromethyl group, a trifluoromethoxy group, an alkyl group having 2 to 10 carbon atoms and substituted with 3 or more fluorine atoms, an alkoxy group having 2 to 10 carbon atoms and substituted with 3 or more fluorine atoms, a fluorine atom or a structure represented by the formula (001).

At least one R¹¹¹ or R¹²¹ is preferably a group substituted with one or more groups selected from the group consisting of a trifluoromethyl group, a trifluoromethoxy group, an alkyl group having 2 to 10 carbon atoms and substituted with 3 or more fluorine atoms, an alkoxy group having 2 to 10 carbon atoms and substituted with 3 or more fluorine atoms, a fluorine atom and a structure represented by the formula (001).

### Specific Example of R¹²¹

R¹²¹ may have a structure, for example, represented above by one of (AAA1) to (AAA126) and (AAC1) to (AAC450).

A material for metal patterning preferably has at least one structure represented by the formula (001) in the molecule. The material for metal patterning more preferably has two or more independent structures represented by the formula (001), still more preferably 3 or more independent structures represented by the formula (001), still more preferably four or more independent structures represented by the formula (001), in the molecule.

The material for metal patterning represented by the formula (A1) or the formula (B1) preferably has at least two groups selected from the group consisting of a heteroaromatic group, a cyclic aliphatic hydrocarbon group and a heteroaliphatic hydrocarbon group in the molecule. Three or more is more preferred, and four or more is still more preferred.

The material for metal patterning preferably has a structure represented by the formula (A1).

The material for metal patterning preferably has a structure represented by the formula (101), (102), (103), (104) or (105). A structure represented by the formula (101), (102), (103) or (104) is more preferred, a structure represented by the formula (101), (102) or (104) is still more preferred, a structure represented by the formula (101) or the formula (104) is still more preferred, and a structure represented by the formula (104) is particularly preferred.

The material for metal patterning with a structure represented by the formula (101), the formula (102), the formula (103), the formula (104) or the formula (105) preferably has at least two groups selected from the group consisting of a heteroaromatic group, a cyclic aliphatic hydrocarbon group and a heteroaliphatic hydrocarbon group in the molecule. Three or more is more preferred, and four or more is still more preferred.

### [Heterocyclic Compound]

A heterocyclic compound according to an embodiment of the present disclosure is represented by the formula (301) or (302):

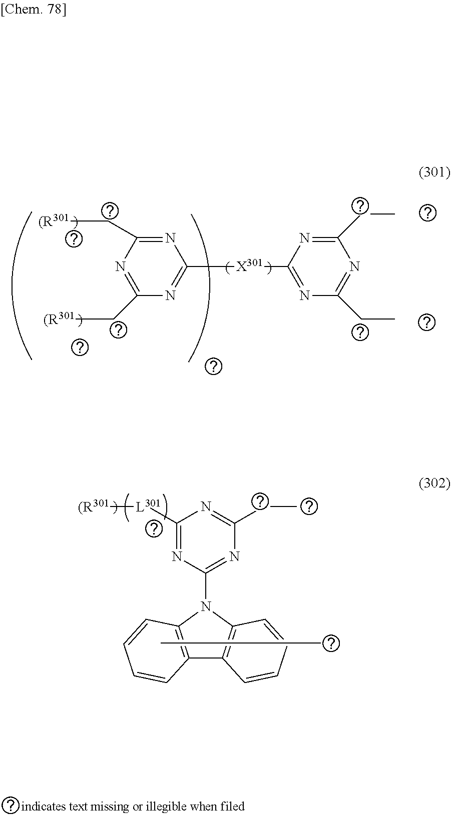

L³⁰¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O, S or NR³⁰¹.

L³⁰³ each independently denotes
a linear, branched or cyclic divalent to tetravalent aliphatic hydrocarbon group with 1 to 18 carbon atoms optionally substituted with fluorine, or
O or S.

Rf³⁰¹ each independently denotes
a moiety with 2 or more carbon atoms containing 3 or more fluorine atoms.

R³⁰¹ each independently denotes
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted cyclic monovalent aliphatic hydrocarbon group with 3 to 18 carbon atoms, or
a fluoroalkyl structure with 1 or more carbon atoms containing 3 or more fluorine atoms.

X³⁰¹ denotes
an optionally substituted monocyclic, linked or condensed divalent to tetravalent aromatic hydrocarbon group with 3 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or O.

gg each independently denotes an integer in the range of 1 to 6.

hh each independently denotes an integer in the range of 0 to 12.

ii each independently denotes an integer in the range of 1 to 6.

jj each independently denotes an integer in the range of 0 to 6.

kk each independently denotes an integer in the range of 1 to 6.

mm each independently denotes an integer in the range of 1 to 6.

nn each independently denotes an integer in the range of 1 to 6.

In the formula (301), when L³⁰¹ is NR³⁰¹, and R³⁰¹ is an optionally substituted monocyclic, linked or condensed monovalent aromatic hydrocarbon group with 6 to 26 carbon atoms, L³⁰¹ is each independently substituted with a fluorine atom or a moiety with 1 or more carbon atoms containing 3 or more fluorine atoms.

When X³⁰¹ denotes a p-phenylene group and nn is 1, the p-phenylene group is substituted with a fluorine atom or a monovalent substituent with 1 or more carbon atoms optionally containing 3 or more fluorine atoms.

### [Rf³⁰¹]

Rf³⁰¹ each independently denotes a moiety with 2 or more carbon atoms containing 3 or more fluorine atoms.

In Rf³⁰¹, the ratio of the number of fluorine atoms to the number of carbon atoms is preferably 50% or more, more preferably 60% or more, still more preferably 70% or more, still more preferably 80% or more.

Rf³⁰¹ may have any molecular structure and may be linear, branched or cyclic.

ii each independently denotes an integer in the range of 1 to 6. ii preferably denotes an integer in the range of 1 to 5, more preferably an integer in the range of 1 to 4, still more preferably an integer in the range of 1 to 3, still more preferably 1 or 2.

Rf³⁰¹ preferably has a structure represented by the following formula (311):

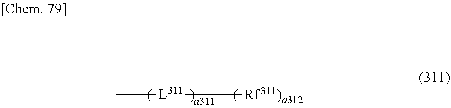

wherein
Rf³¹¹ each independently denotes
a linear, branched or cyclic aliphatic hydrocarbon group with 1 to 18 carbon atoms optionally substituted with a fluorine atom;
L³¹¹ each independently denotes
a linear, branched or cyclic divalent aliphatic hydrocarbon group with 1 to 18 carbon atoms optionally substituted with O, S or a fluorine atom, or
a vinylene group that may form a ring;
a311 each independently denotes an integer in the range of 0 to 4;
a312 each independently denotes an integer in the range of 1 to 4; and
in each Rf³¹¹, the ratio of the number of carbon atoms directly bound to a fluorine atom to the number of carbon atoms constituting the structure is preferably 30% or more, more preferably 40% or more, still more preferably 50% or more, still more preferably 60% or more, particularly preferably 70% or more.

Rf³¹¹ is each independently denotes
a methyl group, an ethyl group, a n-propyl group, a n-butyl group, a n-pentyl group, a n-hexyl group, a n-heptyl group, a n-octyl group, an alkyl group having structural isomerism with these alkyl groups, a cyclopropyl group, a cyclobutyl group, a cyclopentyl group, a 1-methylcyclopentyl group, a cyclopentylmethyl group, a cyclohexyl group or an adamantyl group, each of which may be an optionally substituted.

From the perspective of being able to suppress the formation of a metal film on a film surface, L³¹¹ more preferably each independently denotes
O, S or
a methyl group, an ethyl group, a n-propyl group, a n-butyl group, a n-pentyl group, a n-hexyl group, a n-heptyl group, a n-octyl group, an alkyl group having structural isomerism with these alkyl groups, a cyclopropyl group, a cyclobutyl group, a cyclopentyl group, a 1-methylcyclopentyl group, a cyclopentylmethyl group, a cyclohexyl group or an adamantyl group, each of which may be substituted with a fluorine atom, or
a vinylene group that may form a ring.

a311 preferably each independently ranges from 0 to 3, more preferably 0 to 2.

a312 preferably each independently ranges from 1 to 3, more preferably 1 to 2. The number of carbon atoms in the moiety represented by the formula (311) is preferably 1 or more and 36 or less, more preferably 1 or more and 30 or less, still more preferably 1 or more and 24 or less, still more preferably 1 or more and 20 or less, particularly preferably 1 or more and 16 or less.

The number of carbon atoms in Rf³⁰¹ is 2 or more. The number of carbon atoms in Rf³⁰¹ is preferably 2 or more and 36 or less, more preferably 2 or more and 30 or less, still more preferably 2 or more and 24 or less, still more preferably 2 or more and 20 or less, particularly preferably 2 or more and 16 or less.

### Specific Example of Rf³⁰¹

Rf³⁰¹ may have a structure, for example, represented by one of the following (AAA1) to (AAA126).

The mark “*” in the following structures denotes a binding site.

### [L³⁰³]

L³⁰³ each independently denotes
a linear, branched or cyclic divalent to tetravalent aliphatic hydrocarbon group with 1 to 18 carbon atoms optionally substituted with fluorine, or
O or S.

From the perspective of being able to suppress the formation of a metal film on a film surface, L³⁰³ more preferably each independently denotes
O, S or
a methyl group, an ethyl group, a n-propyl group, a n-butyl group, a n-pentyl group, a n-hexyl group, a n-heptyl group, a n-octyl group, an alkyl group having structural isomerism with these alkyl groups, a cyclopropyl group, a cyclobutyl group, a cyclopentyl group, a 1-methylcyclopentyl group, a cyclopentylmethyl group, a cyclohexyl group or an adamantyl group, each of which may be substituted with a fluorine atom.

hh each independently denotes an integer in the range of 0 to 12. hh preferably denotes an integer in the range of 0 to 10, an integer in the range of 0 to 8, an integer in the range of 0 to 6, an integer in the range of 0 to 4, more preferably an integer in the range of 0 to 2.

### [L³⁰¹]

L³⁰¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O, S or NR³⁰¹.

Preferably, the monocyclic, linked or condensed aromatic hydrocarbon group represented by L³⁰¹ is a phenyl group or has a structure in which a plurality of benzene rings are linked or condensed.

Preferably, the monocyclic, linked or condensed heteroaromatic group represented by L³⁰¹ has N, O or S as a heteroatom and is a 5-membered ring, a 6-membered ring or a structure in which these or a plurality of benzene rings are condensed.

Preferably, the cyclic divalent to tetravalent heteroaliphatic hydrocarbon group represented by L³⁰¹ has N, O or S as a heteroatom and is a 5-membered ring, a 6-membered ring or a condensed structure thereof.

More preferably, a compound that provides the monocyclic, linked or condensed aromatic hydrocarbon group represented by L³⁰¹ is benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene,
a compound that provides the monocyclic, linked or condensed heteroaromatic group represented by L³⁰¹ is pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene,
a compound that provides the linear, branched or cyclic aliphatic hydrocarbon group represented by L³⁰¹ is methane, ethane, propane, butane, adamantane, diamantane, norbornene or cyclohexane, and
a compound that provides the cyclic heteroaliphatic hydrocarbon group represented by L³⁰¹ is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

Still more preferably, the monocyclic, linked or condensed aromatic hydrocarbon group represented by L³⁰¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene or corannulene,
the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole or benzothiazole,
the linear, branched or cyclic aliphatic hydrocarbon group is adamantane, diamantane or cyclohexane, and
a compound that provides the cyclic divalent to tetravalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

When L³⁰¹ denotes a substituted aromatic hydrocarbon group, a substituted heteroaromatic group, a substituted aliphatic hydrocarbon group or a cyclic heteroaliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a fluorine atom, a deuterium atom and a structure represented by the formula (311), and
a combination of these groups.

A substituent of L³⁰¹ is preferably each independently substituted with one or more groups selected from the group consisting of
a methyl group, a trifluoromethyl group, a trifluoromethoxy group, an alkyl group with 2 to 10 carbon atoms, a cyano group, a deuterium atom, a fluorine atom, a phenyl group, a biphenylyl group, a naphthyl group, a phenanthryl group, a pyridyl group, a pyrimidyl group, a pyrazyl group, a triazyl group, an indolyl group, a carbazolyl group, a benzothienyl group, a dibenzothienyl group, a benzofuranyl group, a dibenzofuranyl group, a thiazolyl group, a benzothiazolyl group and a structure represented by the formula (311), and
a combination of these groups.

A substituent of L³⁰¹ is more preferably each independently substituted with one or more groups selected from the group consisting of
a methyl group, a trifluoromethyl group, a trifluoromethoxy group, an alkyl group with 2 to 10 carbon atoms, a fluorine atom, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group, a thiazolyl group and a structure represented by the formula (311), and
a combination of these groups.

A substituent of L³⁰¹ is still more preferably each independently
(iv) a methyl group, a methoxy group, an alkyl group with 2 to 10 carbon atoms, an alkoxy group with 2 to 10 carbon atoms, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group or a thiazolyl group,
(v) the group represented by (iv) substituted with one or more groups selected from the group consisting of a fluorine atom, a perfluoroalkyl group with 1 to 10 carbon atoms and a perfluoroalkoxy group with 1 to 10 carbon atoms, or
(vi) a fluorine atom or a structure represented by the formula (311).

A substituent of L³⁰¹ is still more preferably each independently
(iv′) a methyl group, an ethyl group, a butyl group, a methoxy group, an ethoxy group, a butoxy group, a phenyl group, a naphthyl group, a pyridyl group, a pyrimidyl group, a pyrazyl group, a triazyl group, a carbazolyl group, a dibenzothienyl group, a dibenzofuranyl group or a thiazolyl group,
(v′) the group represented by (iv′) substituted with one or more groups selected from the group consisting of a fluorine atom, a perfluoroalkyl group with 1 to 6 carbon atoms and a perfluoroalkoxy group with 1 to 6 carbon atoms, or
(vi′) a fluorine atom or a structure represented by the formula (311).

A substituent of L³⁰¹ is still more preferably each independently a trifluoromethyl group, a trifluoromethoxy group, an alkyl group having 2 to 10 carbon atoms and substituted with 3 or more fluorine atoms, an alkoxy group having 2 to 10 carbon atoms and substituted with 3 or more fluorine atoms, a structure represented by the formula (311) or a fluorine atom.

### Specific Example of L³⁰¹

L³⁰¹ may have a structure, for example, represented above by one of (AAB1) to (AAB803).

### [R³⁰¹]

R³⁰¹ each independently denotes
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted cyclic monovalent aliphatic hydrocarbon group with 3 to 18 carbon atoms, or
a fluoroalkyl structure with 1 or more carbon atoms containing 3 or more fluorine atoms.

Preferably, the monocyclic, linked or condensed aromatic hydrocarbon group represented by R³⁰¹ is a phenyl group or has a structure in which a plurality of benzene rings are linked or condensed.

Preferably, the monocyclic, linked or condensed heteroaromatic group represented by R³⁰¹ has N, O or S as a heteroatom and is a 5-membered ring, a 6-membered ring or a structure in which these or a plurality of benzene rings are condensed.

More preferably, a compound that provides the monocyclic, linked or condensed monovalent aromatic hydrocarbon group represented by R³⁰¹ is benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene,
a compound that provides the monocyclic, linked or condensed monovalent heteroaromatic group is pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene, and
a compound that provides the cyclic monovalent aliphatic hydrocarbon group is adamantane, diamantane or cyclohexane.

Still more preferably, the monocyclic, linked or condensed aromatic hydrocarbon group represented by R³⁰¹ is phenyl, biphenyl, terphenyl, naphthalene, fluorene, spirobifluorene, 9,9-dimethylfluorene, 9,9-diphenylfluorene, benzofluorene, phenanthrene, fluoranthene or triptycene,
the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole or benzothiazole, and
the cyclic aliphatic hydrocarbon group is adamantane, diamantane or cyclohexane.

Still more preferably, the monocyclic, linked or condensed aromatic hydrocarbon group represented by R³⁰¹ is phenyl, biphenyl, terphenyl, naphthalene, fluorene, spirobifluorene, 9,9-dimethylfluorene, 9,9-diphenylfluorene, benzofluorene, phenanthrene, fluoranthene or triptycene,
the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole or benzothiazole, and
the cyclic aliphatic hydrocarbon group is adamantane, diamantane or cyclohexane.

The fluoroalkyl structure with 1 or more carbon atoms containing 3 or more fluorine atoms represented by R³⁰¹ is preferably a structure represented by the formula (311).

The number of carbon atoms in the fluoroalkyl structure with 1 or more carbon atoms containing 3 or more fluorine atoms represented by R³⁰¹ is preferably 1 or more and 36 or less, more preferably 1 or more and 30 or less, still more preferably 1 or more and 24 or less, still more preferably 1 or more and 20 or less, particularly preferably 1 or more and 16 or less.

When R³⁰¹ is a substituted aromatic hydrocarbon group, a substituted heteroaromatic group or a substituted aliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a hydrogen atom, a fluorine atom, a deuterium atom and a structure represented by the formula (311), and
a combination of these groups.

A substituent of R³⁰¹ is preferably each independently substituted with one or more groups selected from the group consisting of
a methyl group, a trifluoromethyl group, a trifluoromethoxy group, an alkyl group with 2 to 10 carbon atoms, a cyano group, a deuterium atom, a fluorine atom, a phenyl group, a biphenylyl group, a naphthyl group, a phenanthryl group, a pyridyl group, a pyrimidyl group, a pyrazyl group, a triazyl group, an indolyl group, a carbazolyl group, a benzothienyl group, a dibenzothienyl group, a benzofuranyl group, a dibenzofuranyl group, a thiazolyl group, a benzothiazolyl group and a structure represented by the formula (311), and
a combination of these groups.

A substituent of R³⁰¹ is more preferably each independently substituted with one or more groups selected from the group consisting of
a methyl group, a trifluoromethyl group, a trifluoromethoxy group, an alkyl group with 2 to 10 carbon atoms, a fluorine atom, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group, a thiazolyl group and a structure represented by the formula (311), and
a combination of these groups.

A substituent of R³⁰¹ still more preferably each independently is
(vii) a methyl group, a methoxy group, an alkyl group with 2 to 10 carbon atoms, an alkoxy group with 2 to 10 carbon atoms, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group or a thiazolyl group,
(viii) the group represented by (vii) substituted with one or more groups selected from the group consisting of a fluorine atom, a perfluoroalkyl group with 1 to 10 carbon atoms and a perfluoroalkoxy group with 1 to 10 carbon atoms, or
(ix) a fluorine atom or a structure represented by the formula (311).

A substituent of R³⁰¹ still more preferably each independently is
(vii′) a methyl group, an ethyl group, a butyl group, a methoxy group, an ethoxy group, a butoxy group, a phenyl group, a naphthyl group, a pyridyl group, a pyrimidyl group, a pyrazyl group, a triazyl group, a carbazolyl group, a dibenzothienyl group, a dibenzofuranyl group or a thiazolyl group,
(viii′) the group represented by (vii′) substituted with one or more groups selected from the group consisting of a fluorine atom, a perfluoroalkyl group with 1 to 6 carbon atoms and a perfluoroalkoxy group with 1 to 6 carbon atoms, or
(ix′) a fluorine atom or a structure represented by the formula (311).

A substituent of R³⁰¹ preferably each independently denotes
a trifluoromethyl group, a trifluoromethoxy group, an alkyl group having 2 to 10 carbon atoms and substituted with 3 or more fluorine atoms, an alkoxy group having 2 to 10 carbon atoms and substituted with 3 or more fluorine atoms, a fluorine atom or a structure represented by the formula (311).

### Specific Example of R³⁰¹

R³⁰¹ may have a structure, for example, represented above by one of (AAA1) to (AAA126) and (AAC1) to (AAC450).

### [X³⁰¹]

X³⁰¹ denotes
an optionally substituted monocyclic, linked or condensed divalent to tetravalent aromatic hydrocarbon group with 3 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O.

Preferably, the monocyclic, linked or condensed aromatic hydrocarbon group represented by X³⁰¹ is a phenyl group or has a structure in which a plurality of benzene rings are linked or condensed.

Preferably, the monocyclic, linked or condensed heteroaromatic group represented by X³⁰¹ has N, O or S as a heteroatom and is a 5-membered ring, a 6-membered ring or a structure in which these or a plurality of benzene rings are condensed.

Preferably, the cyclic heteroaliphatic hydrocarbon group represented by X³⁰¹ has N, O or S as a heteroatom and is a 5-membered ring, a 6-membered ring or a condensed structure thereof.

More preferably, a compound that provides the monocyclic, linked or condensed aromatic hydrocarbon group represented by X³⁰¹ is benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene, corannulene or one of these groups condensed with one or more selected from the group consisting of benzene, naphthalene and phenanthrene,
a compound that provides the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole, thiadiazole, thianthrene, acridine, dihydroacridine, phenoxazine, phenothiazine, dibenzo-1,4-dioxin, 5,6,7,8-tetrahydroquinoxaline, 2,3,4,5-tetrahydro-1H-1,4-benzodiazepine, 2,3,4,5-tetrahydro-1H-1,5-benzodiazepine, benzothiazole or one of these groups condensed with one or more selected from the group consisting of benzene, naphthalene and phenanthrene,
a compound that provides the cyclic aliphatic hydrocarbon group is adamantane, diamantane, norbornene or cyclohexane, and
a compound that provides the cyclic heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

Still more preferably, a compound that provides the monocyclic, linked or condensed aromatic hydrocarbon group represented by X³⁰¹ is benzene, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene, dibenzochrysene, triptycene or corannulene,
a compound that provides the monocyclic, linked or condensed heteroaromatic group is pyridine, pyrimidine, pyrazine, triazine, carbazole, furan, thiophene, benzofuran, benzothiophene, dibenzofuran, dibenzothiophene, thiazole, 5,6,7,8-tetrahydroquinoxaline, 2,3,4,5-tetrahydro-1H-1,4-benzodiazepine, 2,3,4,5-tetrahydro-1H-1,5-benzodiazepine or benzothiazole,
a compound that provides the cyclic aliphatic hydrocarbon group is adamantane, diamantane or cyclohexane, and
a compound that provides the cyclic divalent to tetravalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

When X³⁰¹ denotes a substituted aromatic hydrocarbon group, a substituted heteroaromatic group, a substituted cyclic aliphatic hydrocarbon group or a substituted cyclic heteroaliphatic hydrocarbon group, these groups are preferably each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a fluorine atom, a deuterium atom and a structure represented by the formula (311), and
a combination of these groups.

A substituent of X³⁰¹ is preferably each independently substituted with one or more groups selected from the group consisting of
a methyl group, a trifluoromethyl group, a trifluoromethoxy group, an alkyl group with 2 to 10 carbon atoms, a cyano group, a deuterium atom, a fluorine atom, a phenyl group, a biphenylyl group, a naphthyl group, a phenanthryl group, a pyridyl group, a pyrimidyl group, a pyrazyl group, a triazyl group, an indolyl group, a carbazolyl group, a benzothienyl group, a dibenzothienyl group, a benzofuranyl group, a dibenzofuranyl group, a thiazolyl group, a benzothiazolyl group and a structure represented by the formula (311), and
a combination of these groups.

A substituent of X³⁰¹ is more preferably each independently substituted with one or more groups selected from the group consisting of
a methyl group, a trifluoromethyl group, a trifluoromethoxy group, an alkyl group with 2 to 10 carbon atoms, a fluorine atom, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group, a thiazolyl group and a structure represented by the formula (311), and
a combination of these groups.

A substituent of X³⁰¹ is still more preferably each independently
(iv) a methyl group, a methoxy group, an alkyl group with 2 to 10 carbon atoms, an alkoxy group with 2 to 10 carbon atoms, a phenyl group, a biphenyl group, a naphthyl group, a phenanthryl group, a pyridyl group, a pyrimidyl group, a pyrazyl group, a triazyl group, a carbazolyl group, a benzothienyl group, a dibenzothienyl group, a benzofuranyl group, a dibenzofuranyl group or a thiazolyl group,
(v) the group represented by (iv) substituted with one or more groups selected from the group consisting of a fluorine atom, a perfluoroalkyl group with 1 to 10 carbon atoms and a perfluoroalkoxy group with 1 to 10 carbon atoms, or
(vi) a fluorine atom or a structure represented by the formula (311).

A substituent of X³⁰¹ is still more preferably each independently
(iv′) a methyl group, an ethyl group, a butyl group, a methoxy group, an ethoxy group, a butoxy group, a phenyl group, a naphthyl group, a pyridyl group, a pyrimidyl group, a pyrazyl group, a triazyl group, a carbazolyl group, a dibenzothienyl group, a dibenzofuranyl group or a thiazolyl group,
(v′) the group represented by (iv′) substituted with one or more groups selected from the group consisting of a fluorine atom, a perfluoroalkyl group with 1 to 6 carbon atoms and a perfluoroalkoxy group with 1 to 6 carbon atoms, or
(vi′) a fluorine atom or a structure represented by the formula (311).

A substituent of X³⁰¹ is still more preferably each independently a trifluoromethyl group, a trifluoromethoxy group, an alkyl group having 2 to 10 carbon atoms and substituted with 3 or more fluorine atoms, an alkoxy group having 2 to 10 carbon atoms and substituted with 3 or more fluorine atoms, a structure represented by the formula (311) or a fluorine atom.

When X³⁰¹ denotes a phenylene group and nn is 1, preferably, the phenylene group is substituted with a fluorine atom or a monovalent substituent with 1 or more carbon atoms optionally containing 3 or more fluorine atoms.

When X³⁰¹ denotes a naphthylene group and nn is 1, more preferably, the phenylene group is substituted with a fluorine atom or a monovalent substituent with 1 or more carbon atoms optionally containing 3 or more fluorine atoms.

When X³⁰¹ denotes an optionally substituted monocyclic, linked or condensed divalent aromatic hydrocarbon group with 3 to 26 carbon atoms and nn is 1, X³⁰¹ is substituted with a fluorine atom or a moiety with 1 or more carbon atoms containing 3 or more fluorine atoms. At this time, a substituent of X³⁰¹ is preferably each independently a fluorine atom or a structure represented by the formula (311).

When X³⁰¹ contains one or more optionally substituted monocyclic, linked or condensed divalent aromatic hydrocarbon group with 3 to 26 carbon atoms, at least one X³⁰¹ is preferably each independently substituted with a fluorine atom or a structure represented by the formula (311).

When X³⁰¹ denotes an optionally substituted monocyclic, linked or condensed divalent to tetravalent aromatic hydrocarbon group with 3 to 26 carbon atoms, X³⁰¹ is preferably each independently substituted with a fluorine atom or a structure represented by the formula (311).

X³⁰¹ preferably denotes
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O.

### Specific Example of X³⁰¹

X³⁰¹ may have a structure, for example, represented above by one of (AAB1) to (AAB803).

gg each independently denotes an integer in the range of 1 to 6. gg preferably each independently denotes an integer in the range of 1 to 5, more preferably an integer in the range of 1 to 4, still more preferably an integer in the range of 1 to 3.

hh each independently denotes an integer in the range of 0 to 6. hh preferably each independently denotes an integer in the range of 0 to 5, more preferably an integer in the range of 0 to 4, still more preferably an integer in the range of 0 to 3, still more preferably an integer in the range of 0 to 2.

jj each independently denotes an integer in the range of 1 to 6. jj preferably each independently denotes an integer in the range of 1 to 5, more preferably an integer in the range of 1 to 4, still more preferably an integer in the range of 1 to 3, still more preferably an integer of 1 or 2.

kk each independently denotes an integer in the range of 1 to 6. kk preferably each independently denotes an integer in the range of 1 to 5, more preferably an integer in the range of 1 to 4, still more preferably an integer in the range of 1 to 3, still more preferably an integer of 1 or 2.

mm each independently denotes an integer in the range of 1 to 6. mm preferably each independently denotes an integer in the range of 1 to 5, more preferably an integer in the range of 1 to 4, still more preferably an integer in the range of 1 to 3.

nn preferably each independently denotes an integer in the range of 1 to 6. nn preferably each independently denotes an integer in the range of 1 to 5, more preferably an integer in the range of 1 to 4, still more preferably an integer in the range of 1 to 3, still more preferably an integer of 1 or 2.

In the formula (301), when L³⁰¹ is NR³⁰¹, and R³⁰¹ is an optionally substituted monocyclic, linked or condensed monovalent aromatic hydrocarbon group with 6 to 26 carbon atoms, L³⁰¹ is each independently substituted with a fluorine atom or a moiety with 1 or more carbon atoms containing 3 or more fluorine atoms.

In the formula (301), when R³⁰¹ is an optionally substituted monocyclic, linked or condensed monovalent aromatic hydrocarbon group with 6 to 26 carbon atoms, R³⁰¹ is preferably substituted with a fluorine atom or a moiety with 1 or more carbon atoms containing 3 or more fluorine atoms.

### [Specific Examples of Material for Metal Patterning and Heterocyclic Compound]

A material for metal patterning according to an embodiment of the present disclosure and a heterocyclic compound according to an embodiment of the present disclosure are exemplified by the following compounds (Z1) to (Z563), but the present disclosure is not limited to these compounds.

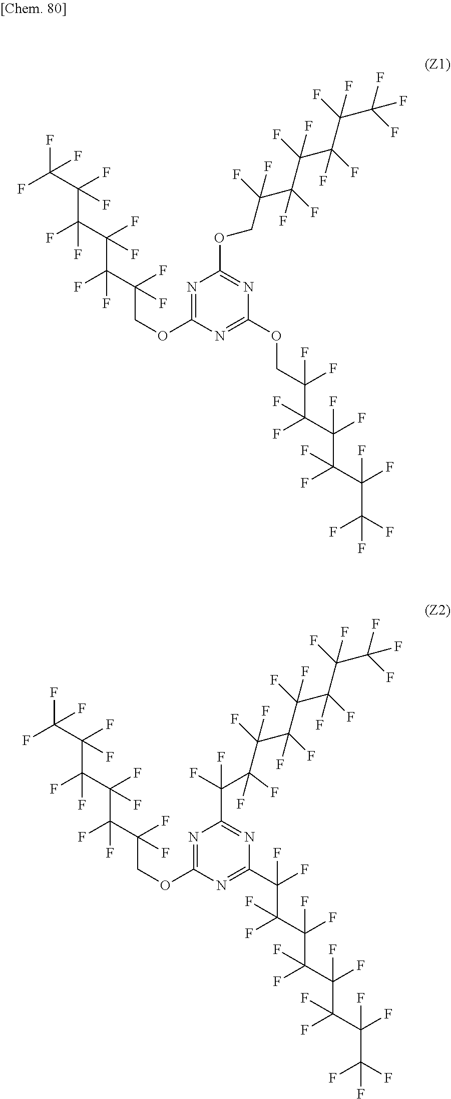 [SMILES: FC(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)F)n1 ; FC(F)(F)C(F)(F)C(F)(F)C(F)(F)C(F)(F)C(F)(F)COc1nc(C(F)(F)C(F)(F)C(F)(F)C(F)(F)C(F)(F)C(F)(F)C(F)(F)C(F)(F)F)nc(C(F)(F)C(F)(F)C(F)(F)C(F)(F)C(F)(F)C(F)(F)C(F)(F)C(F)(F)F)n1]

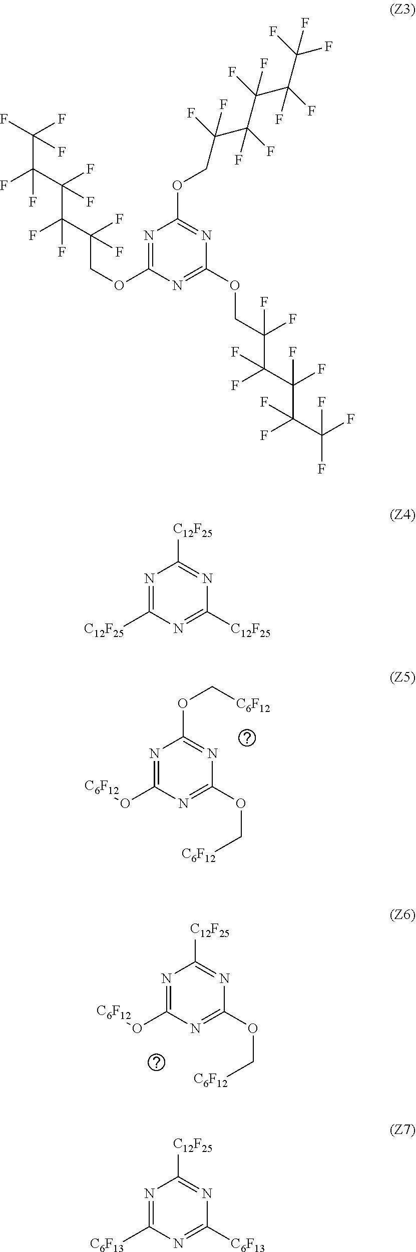

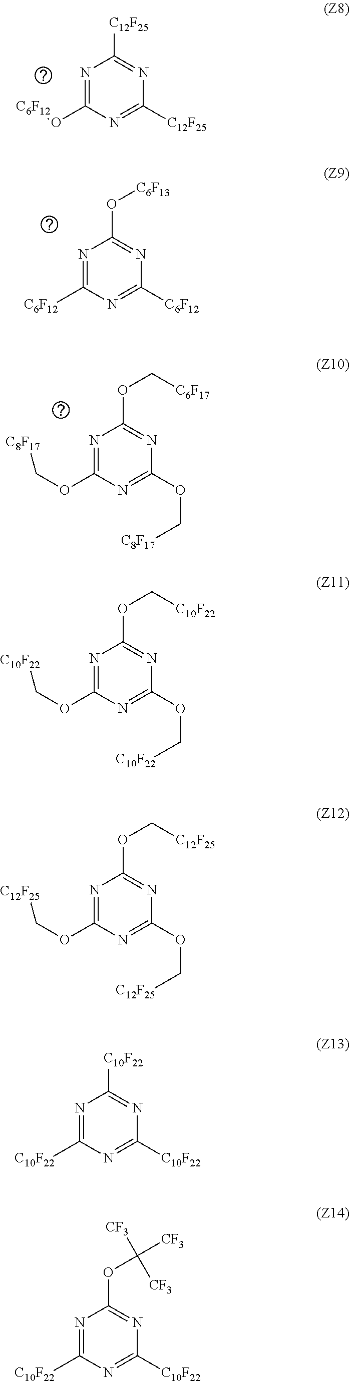

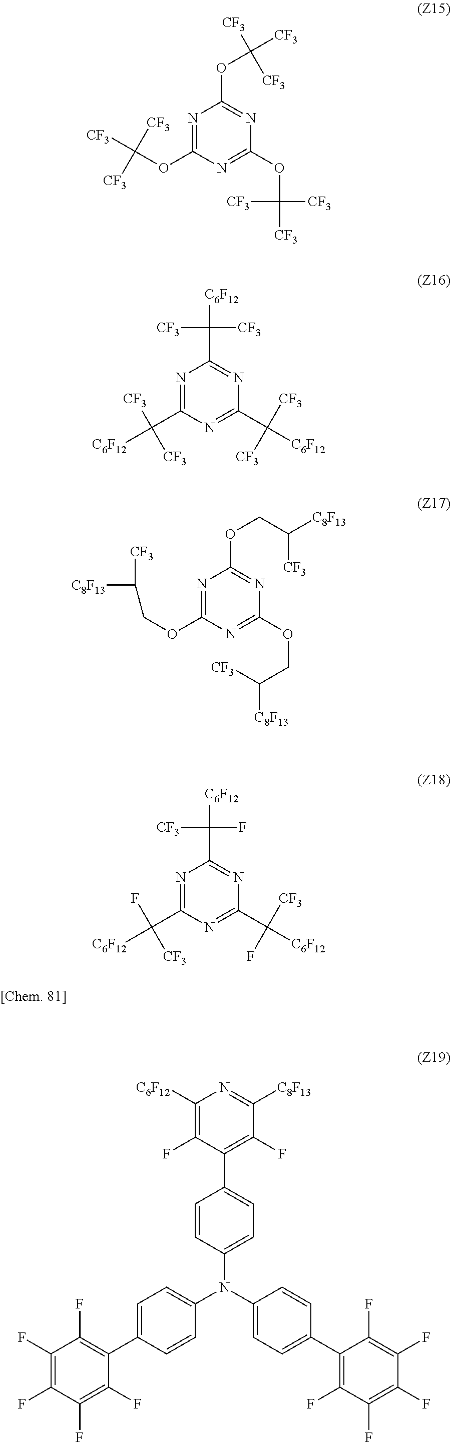

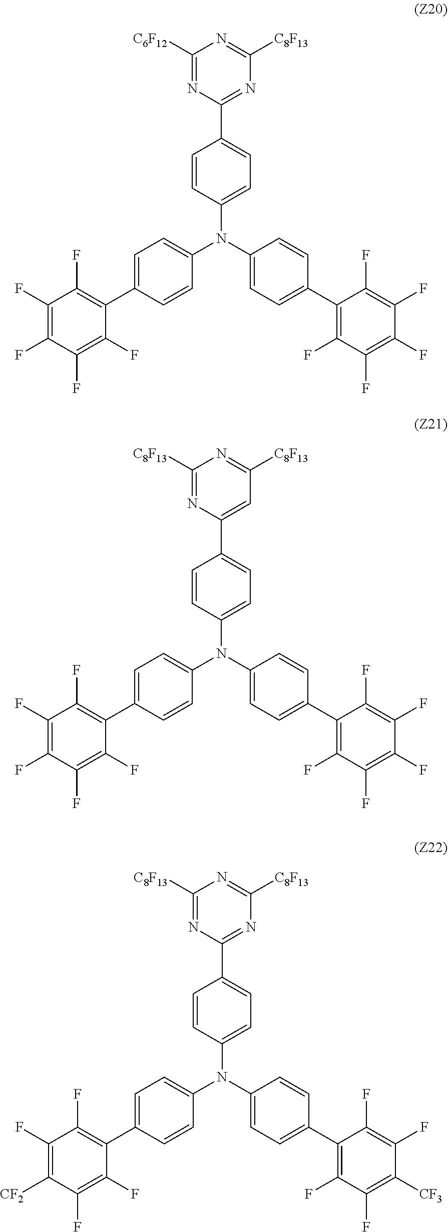

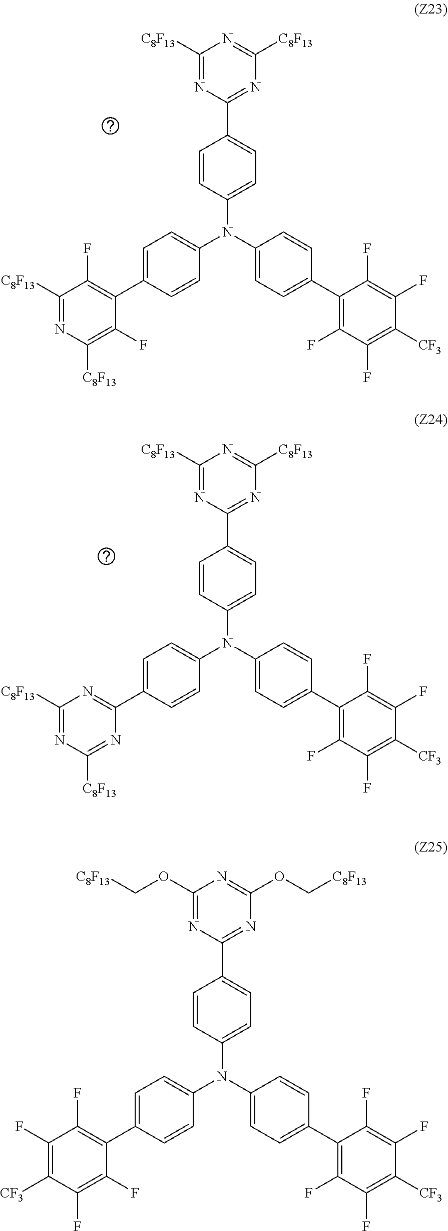

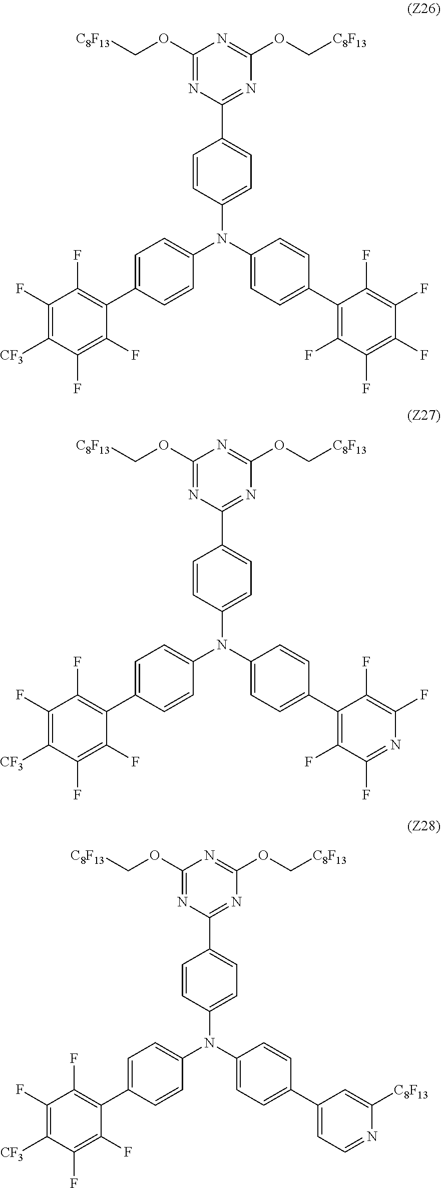

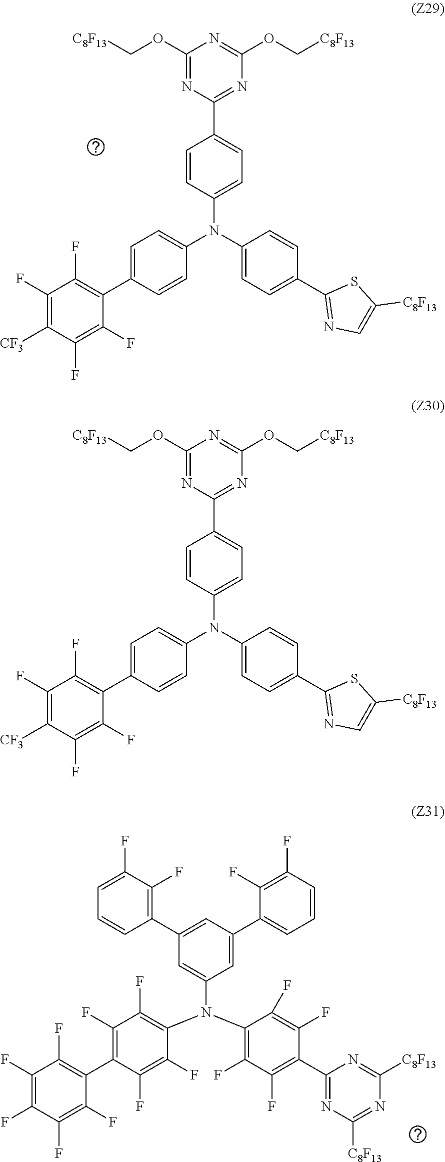

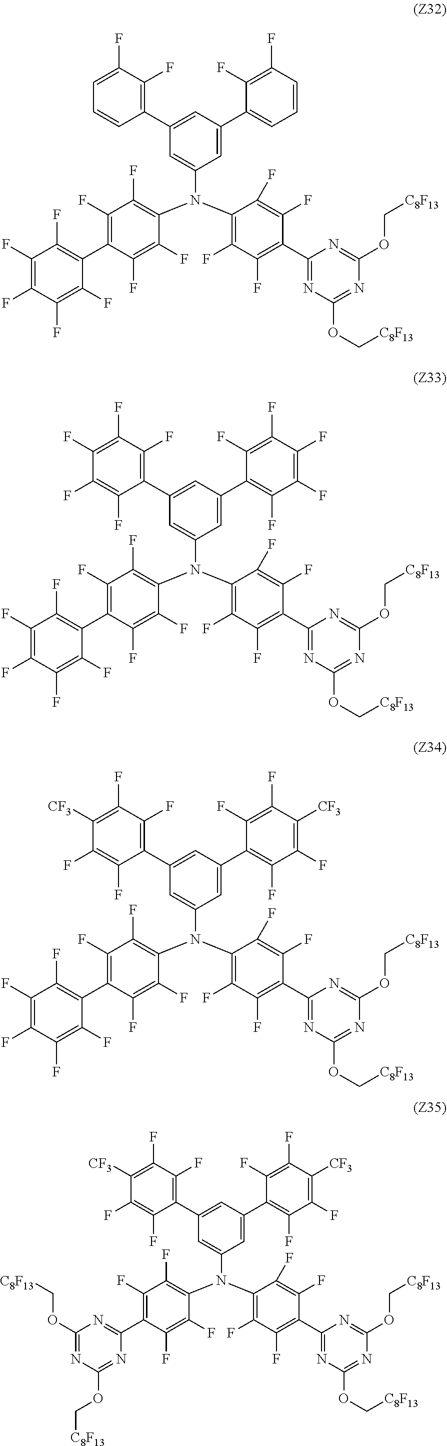

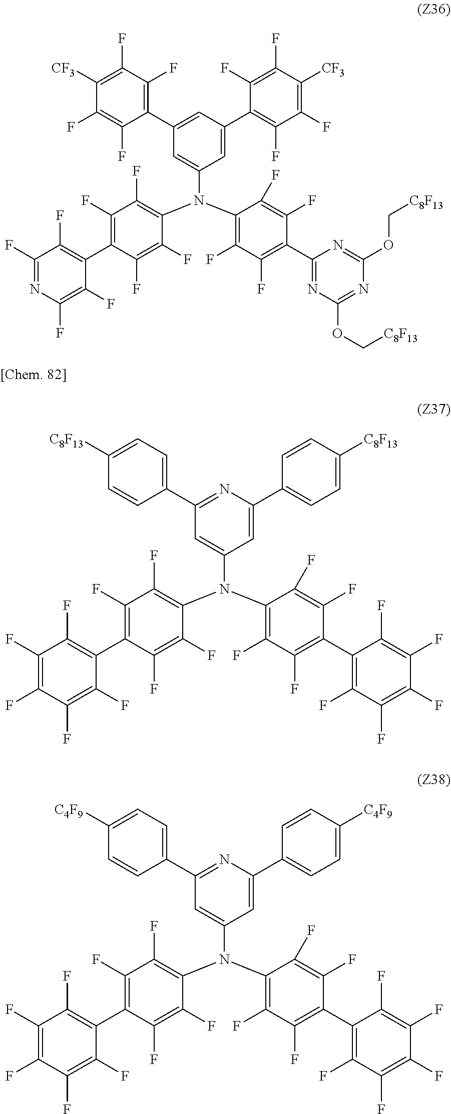

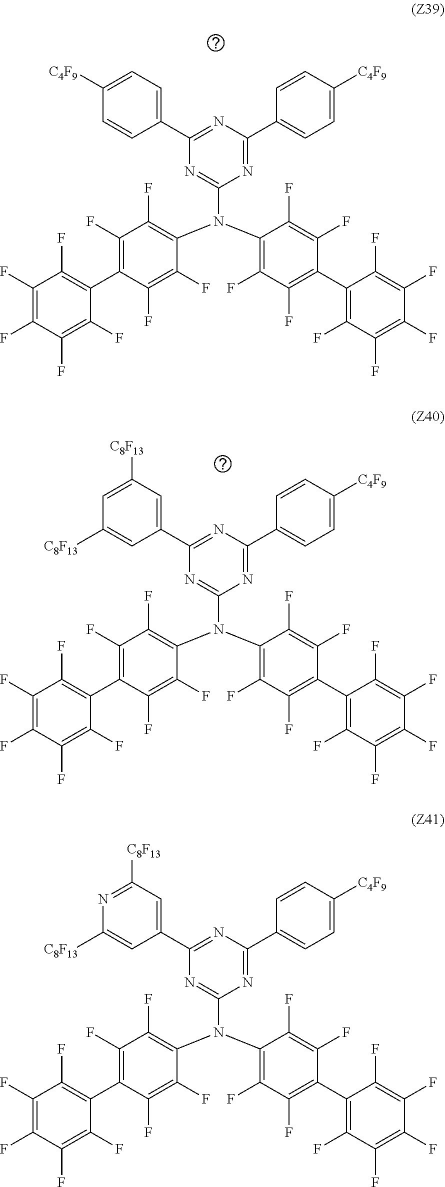

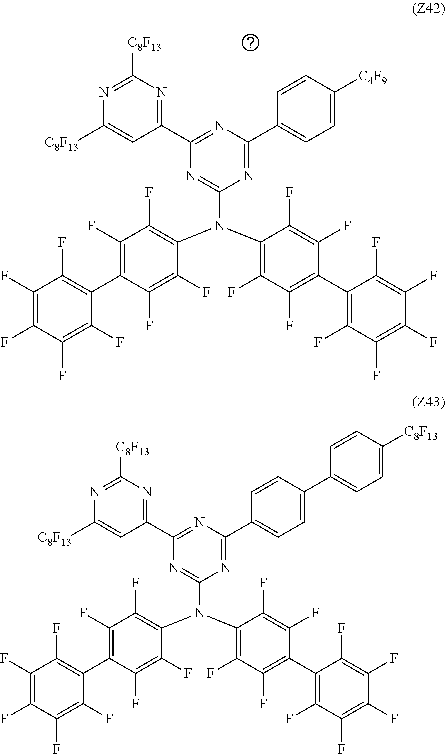

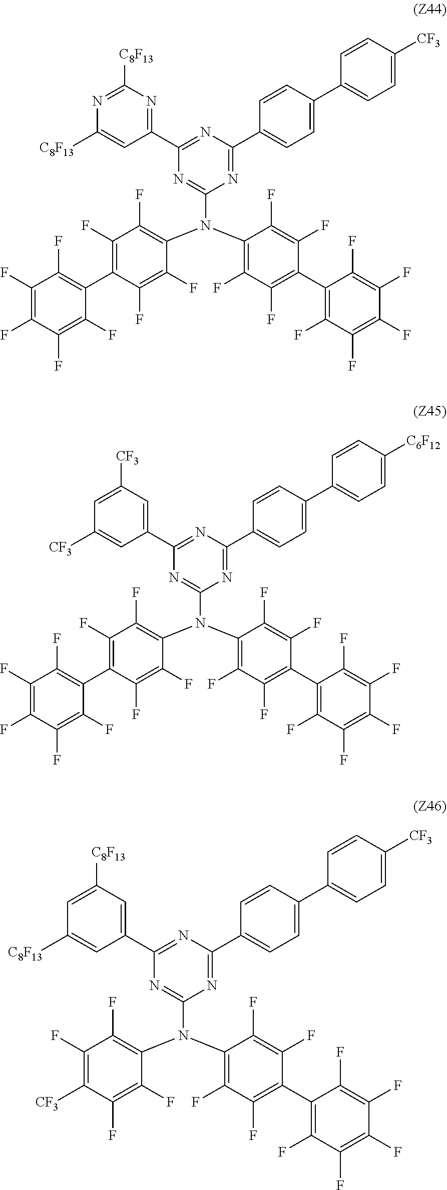

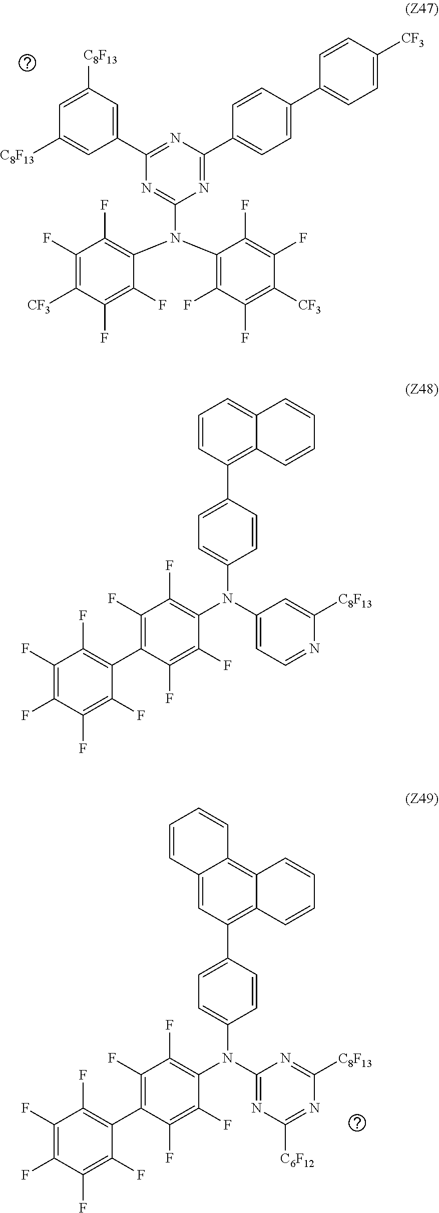

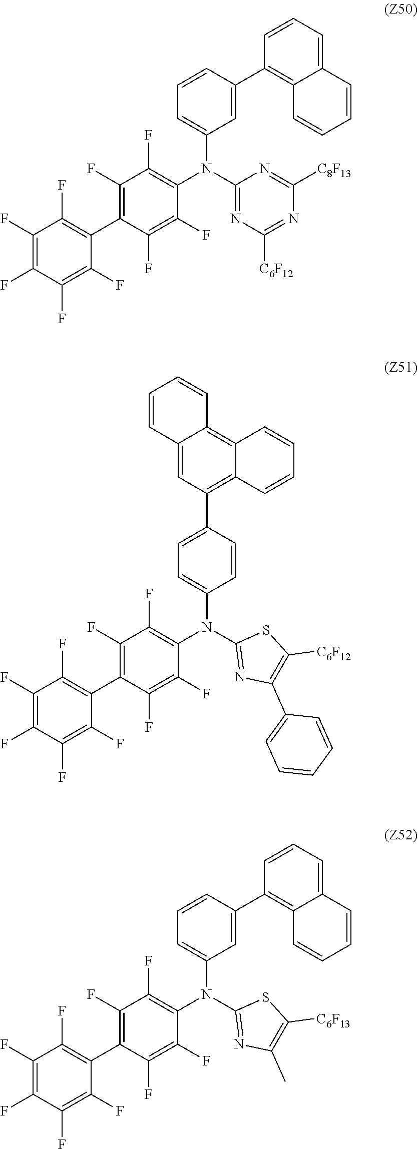

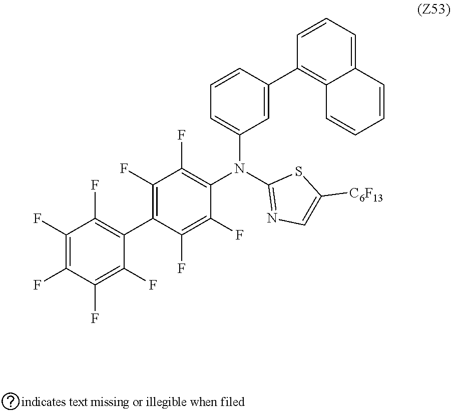

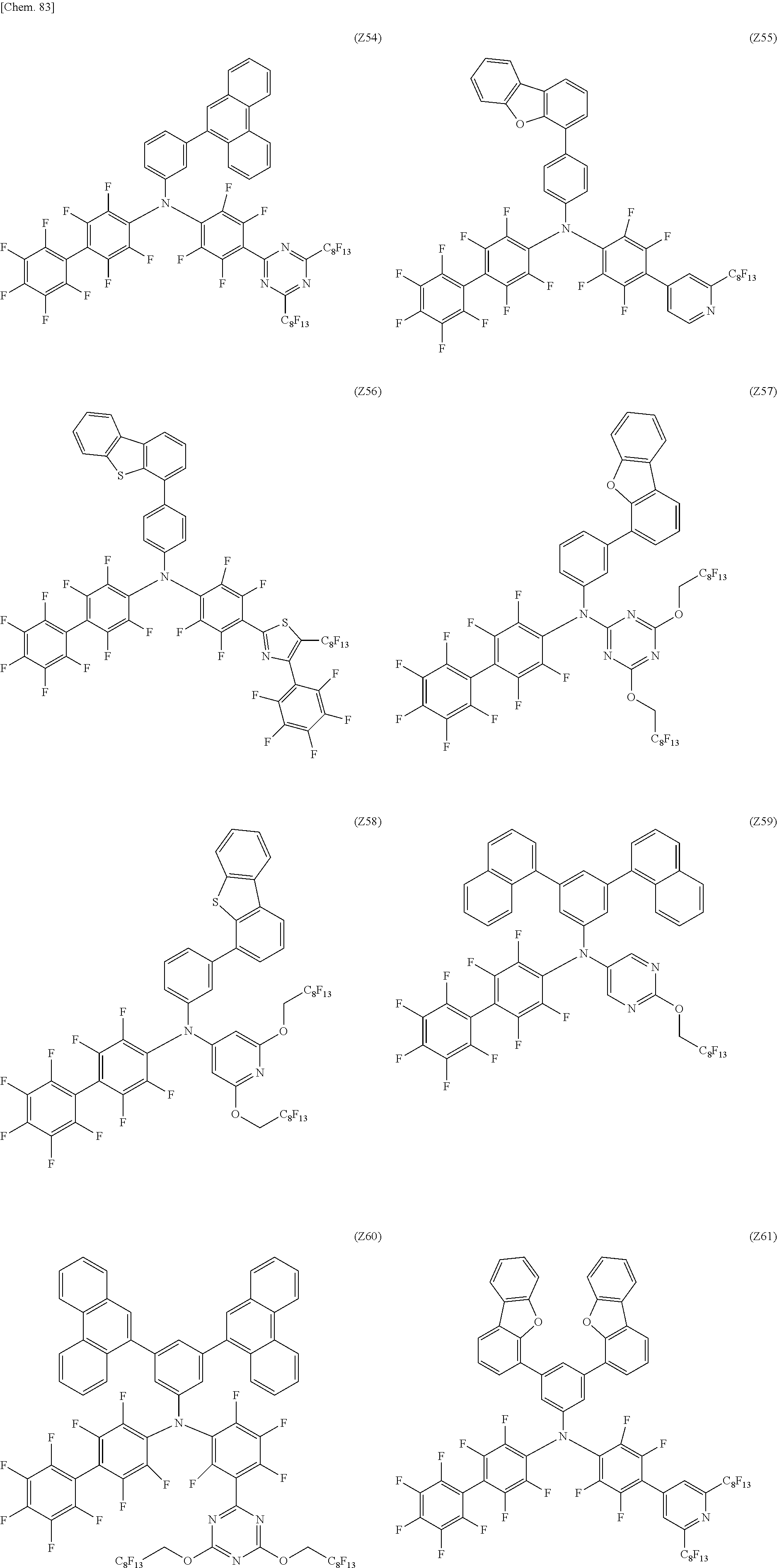

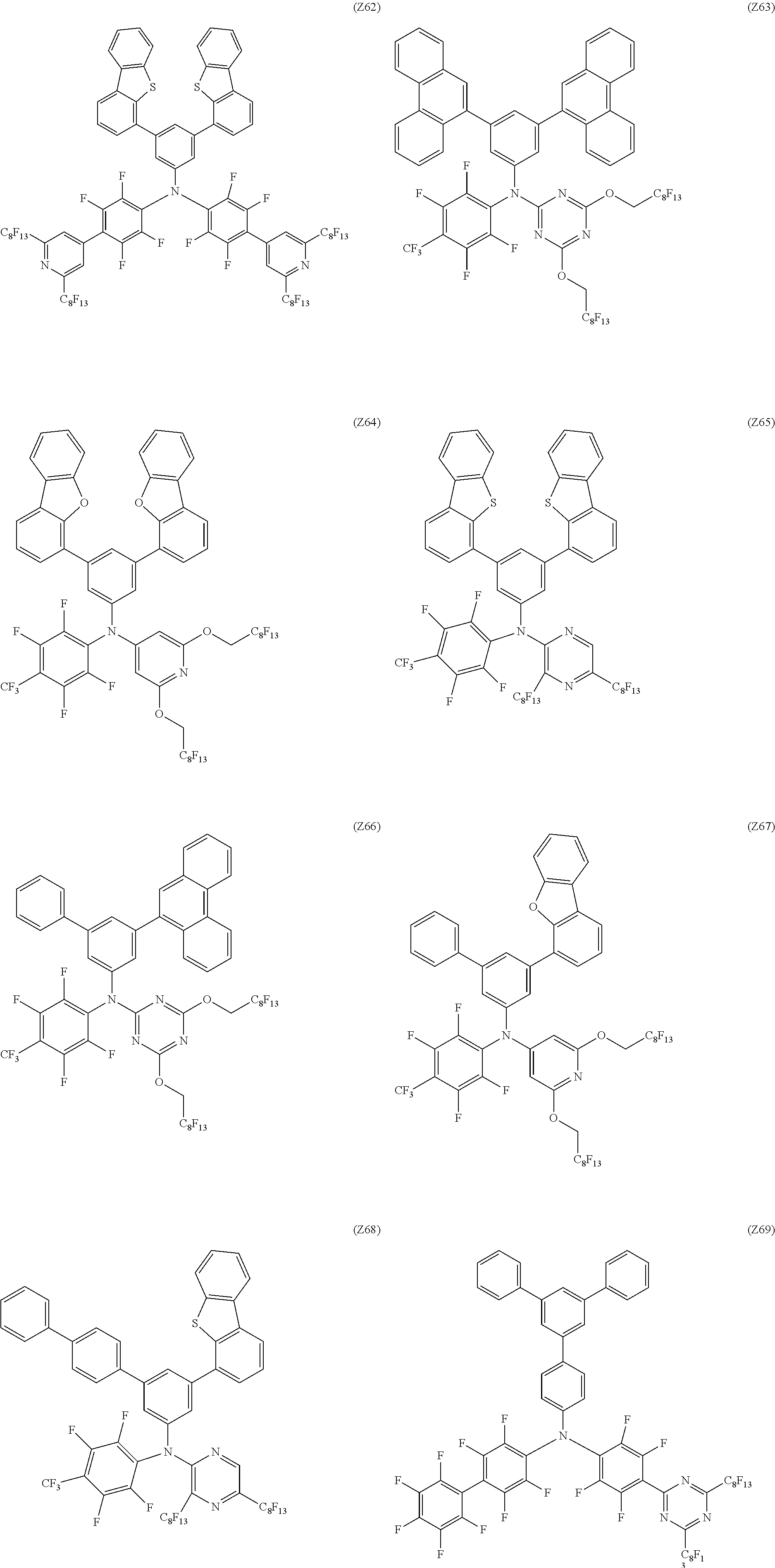

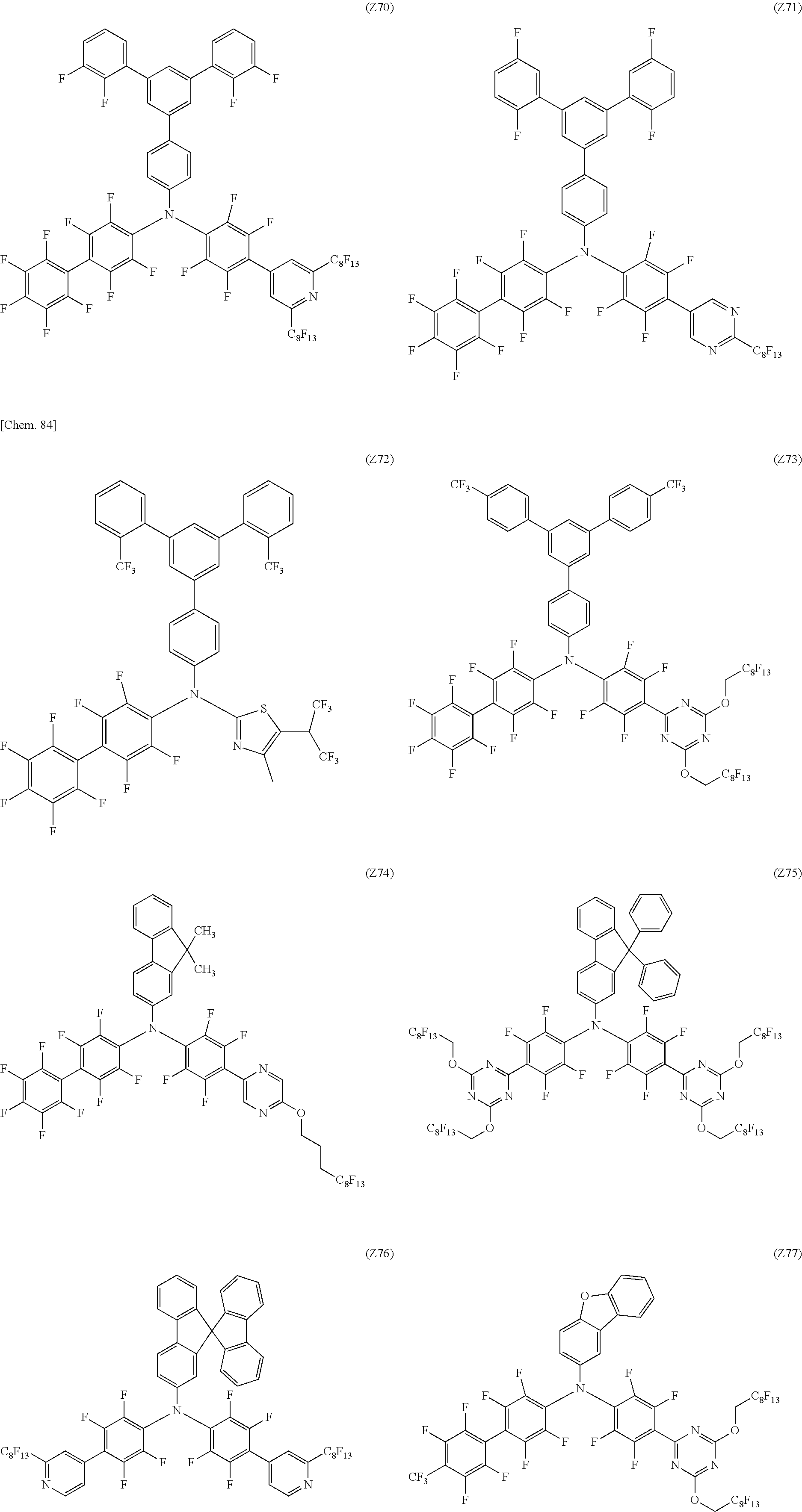

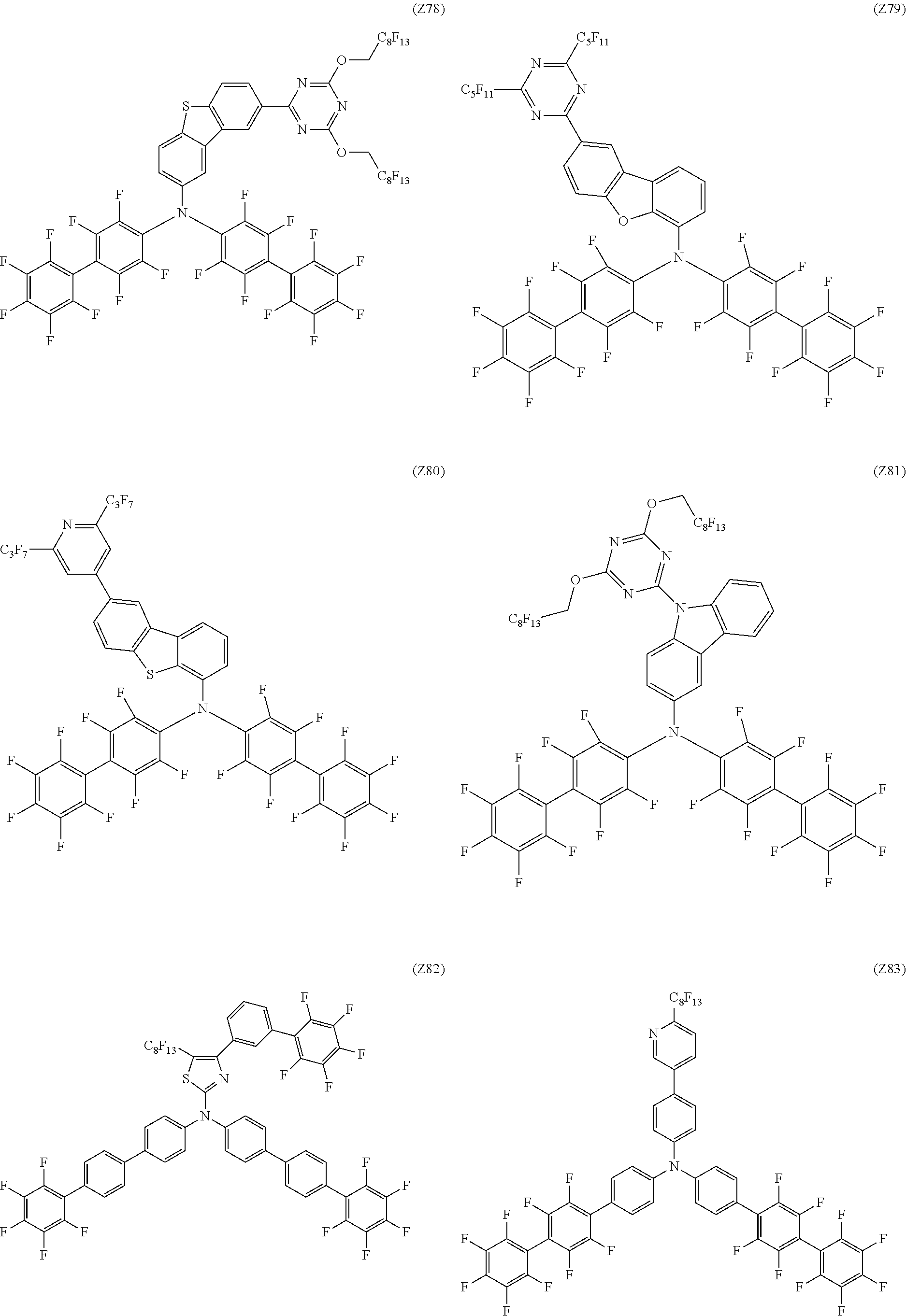

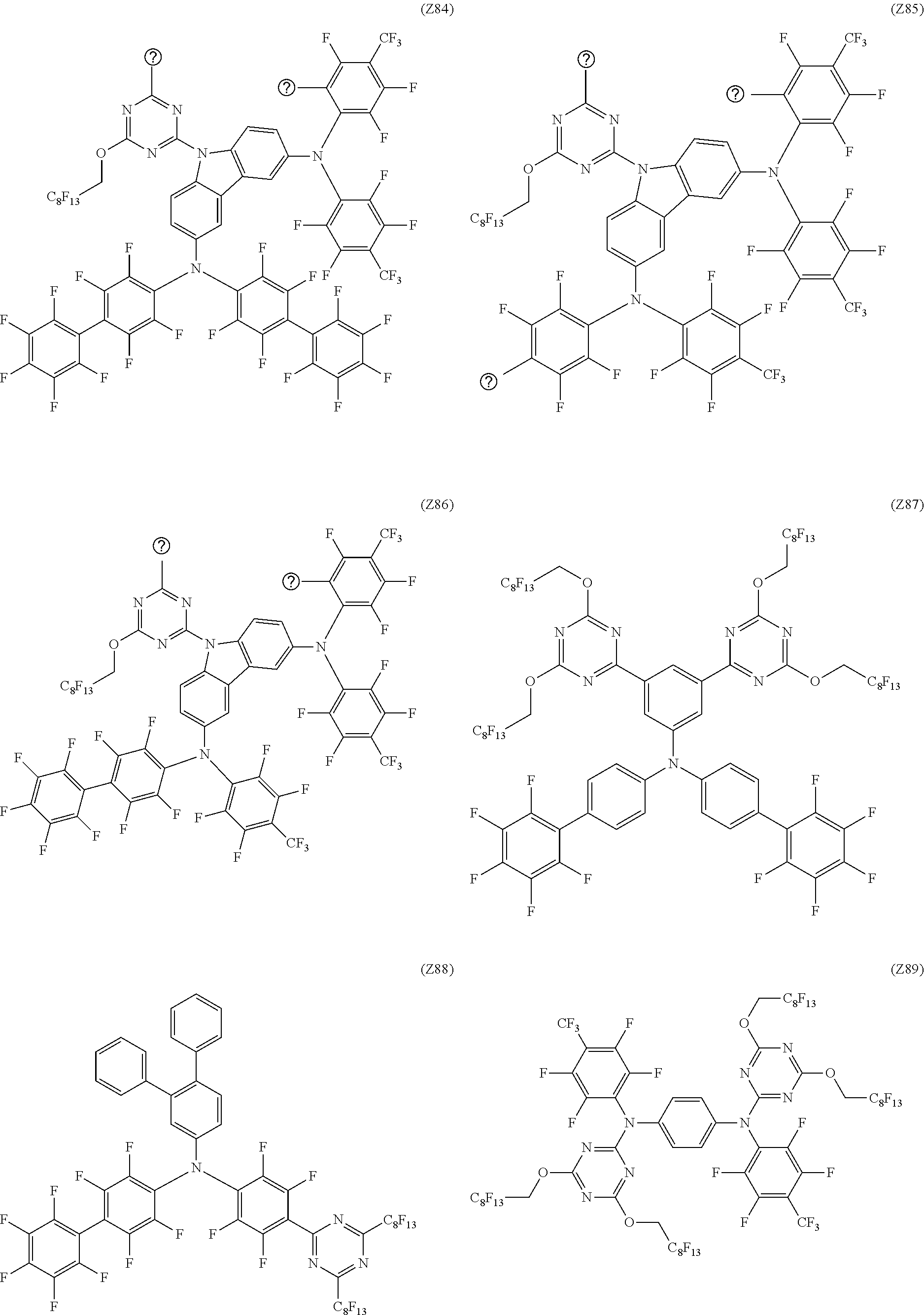

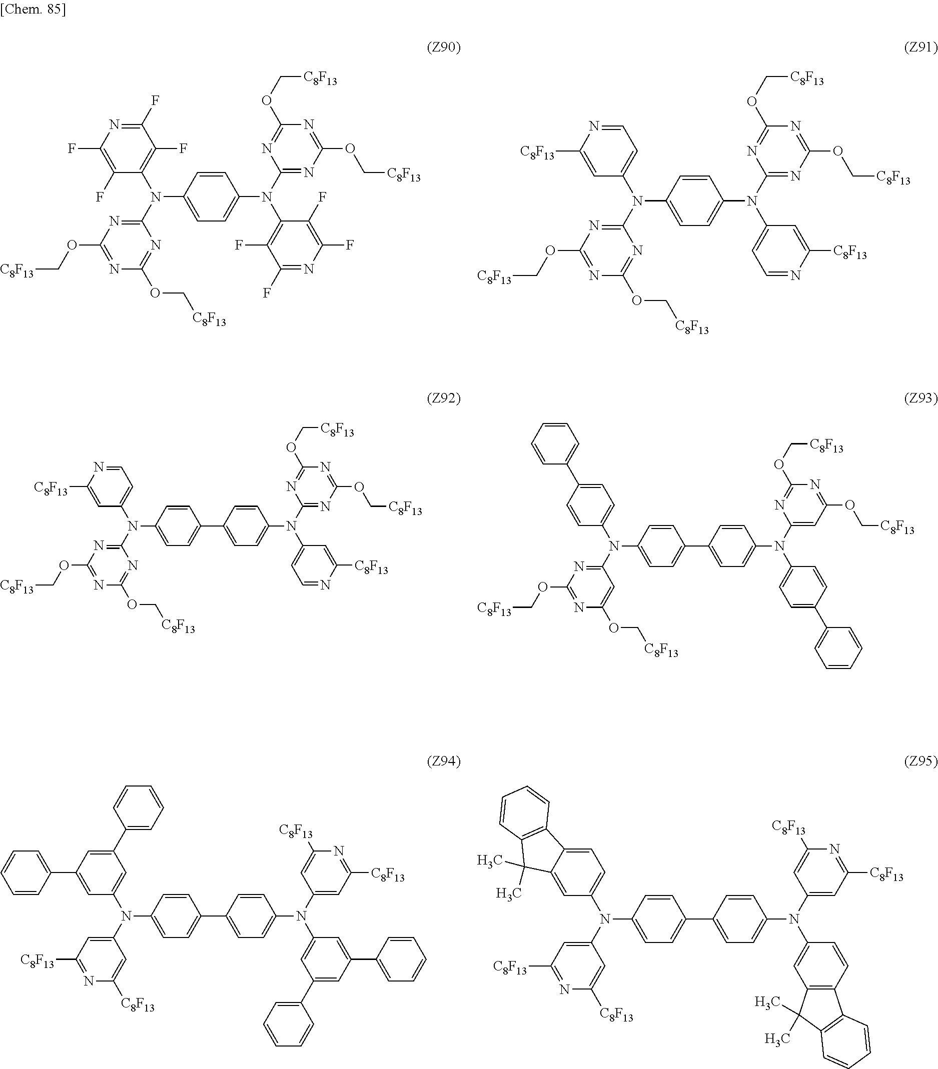

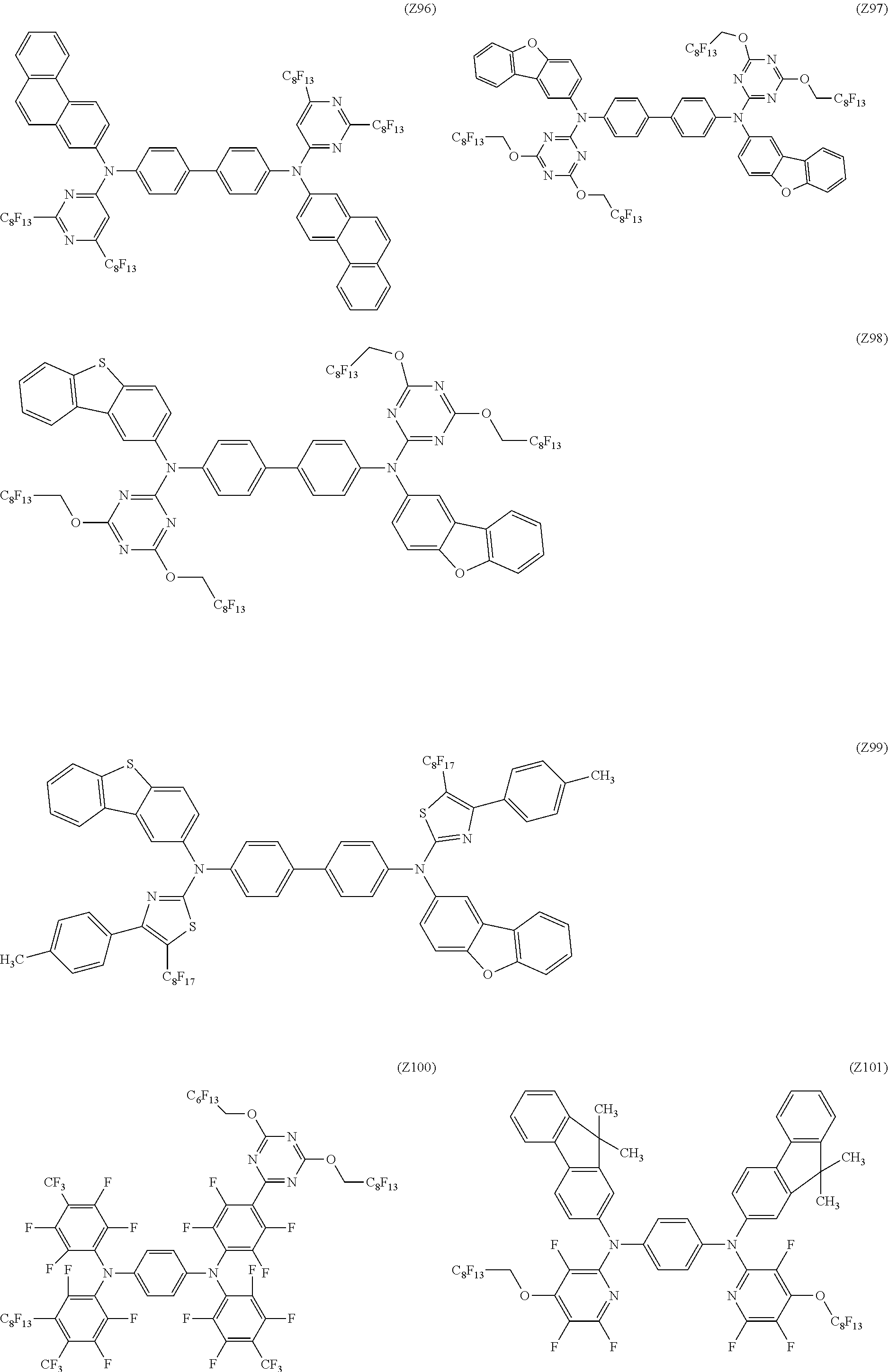

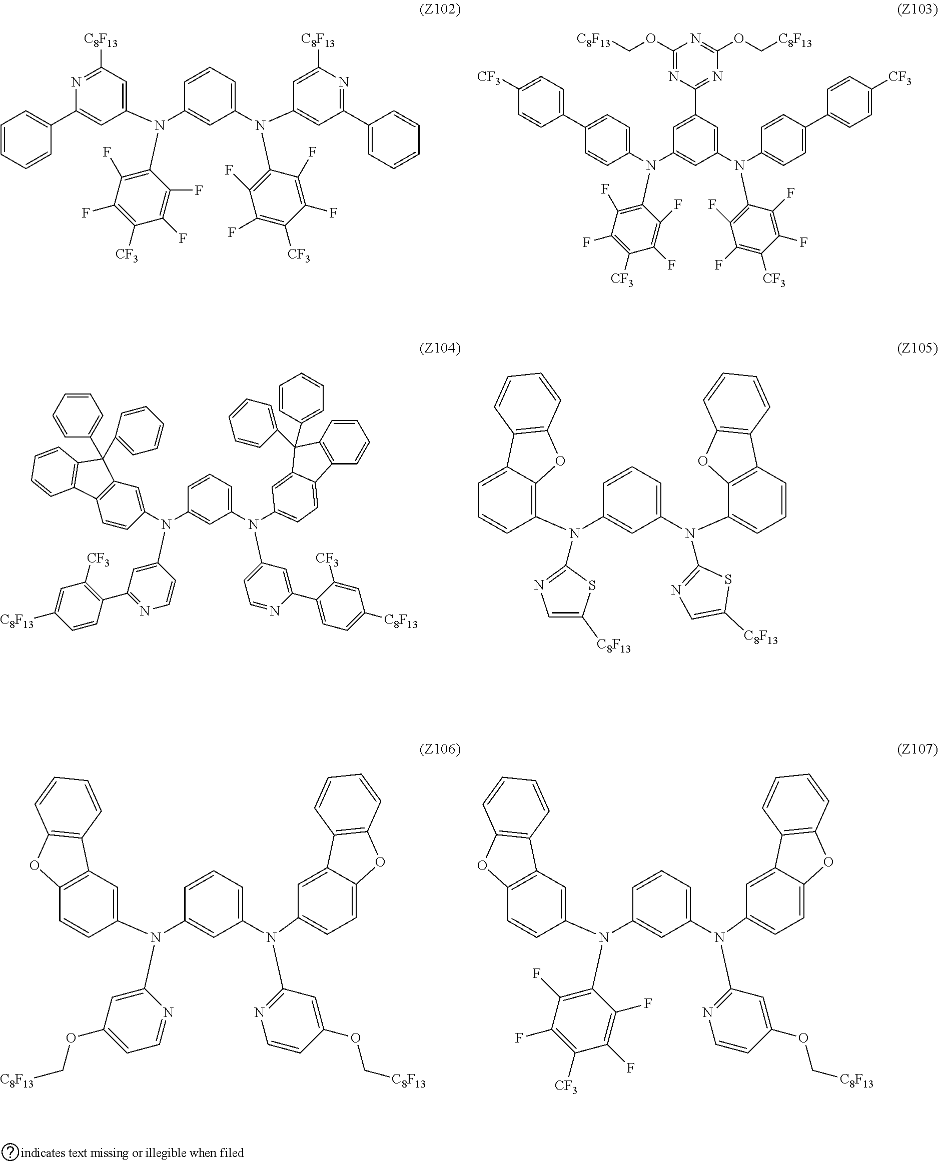

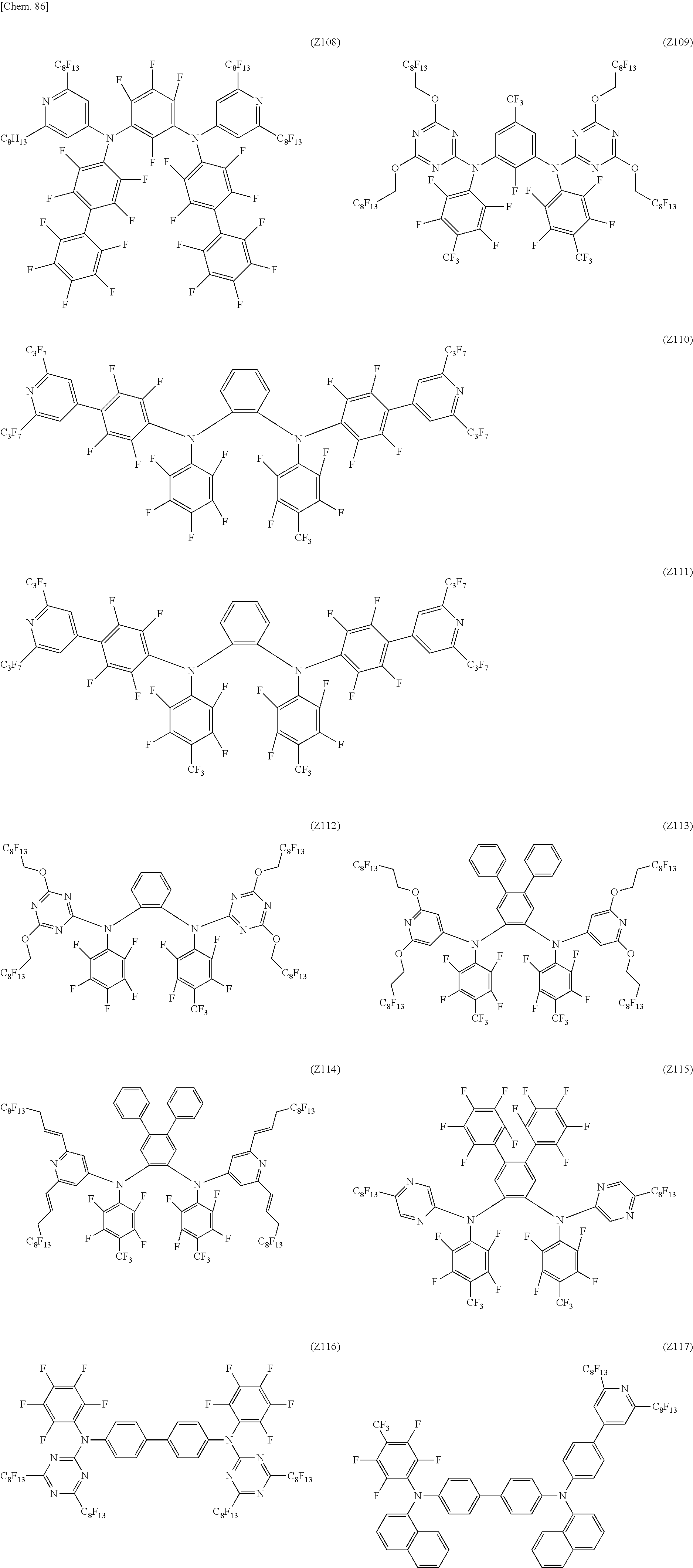

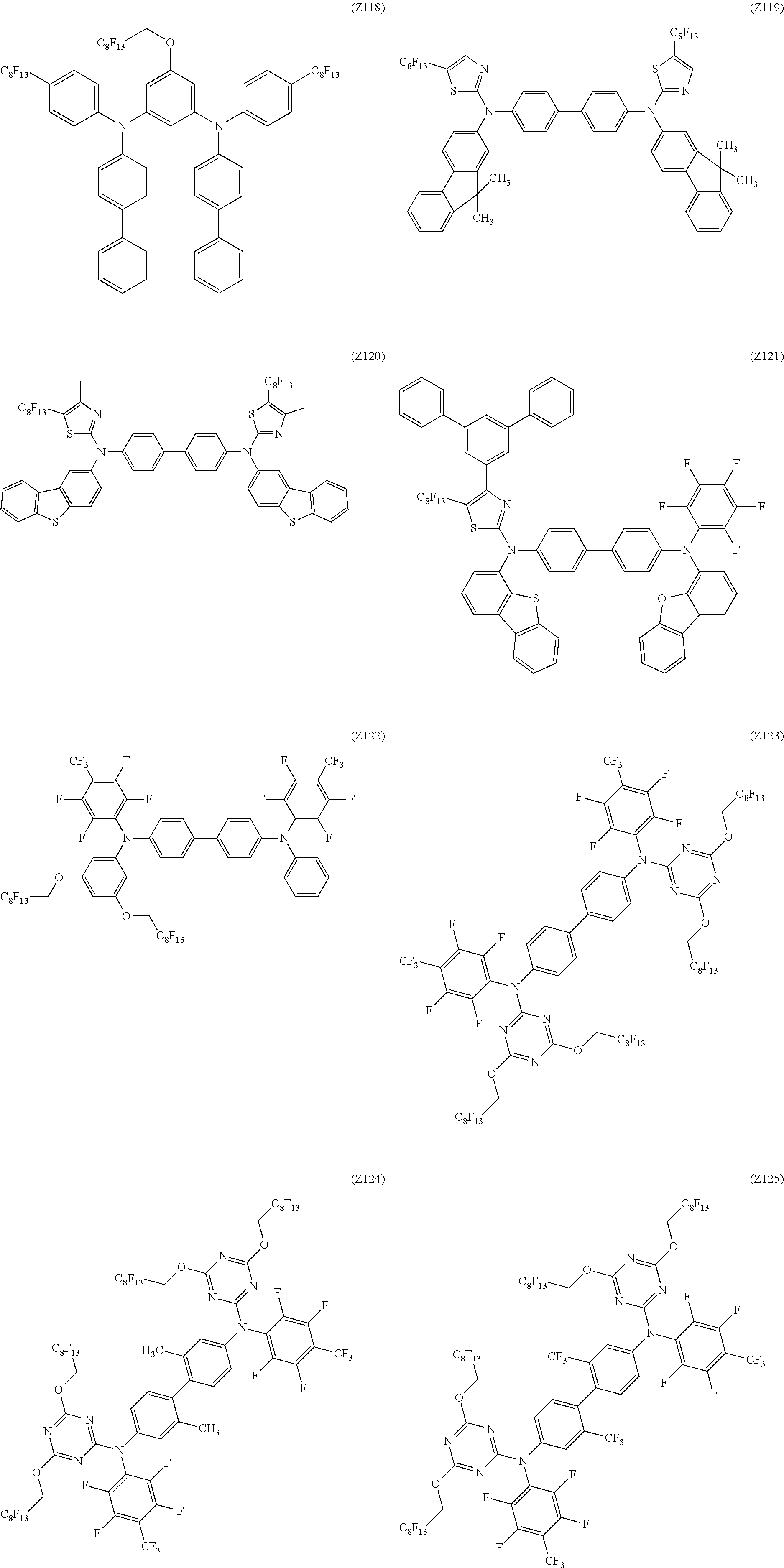

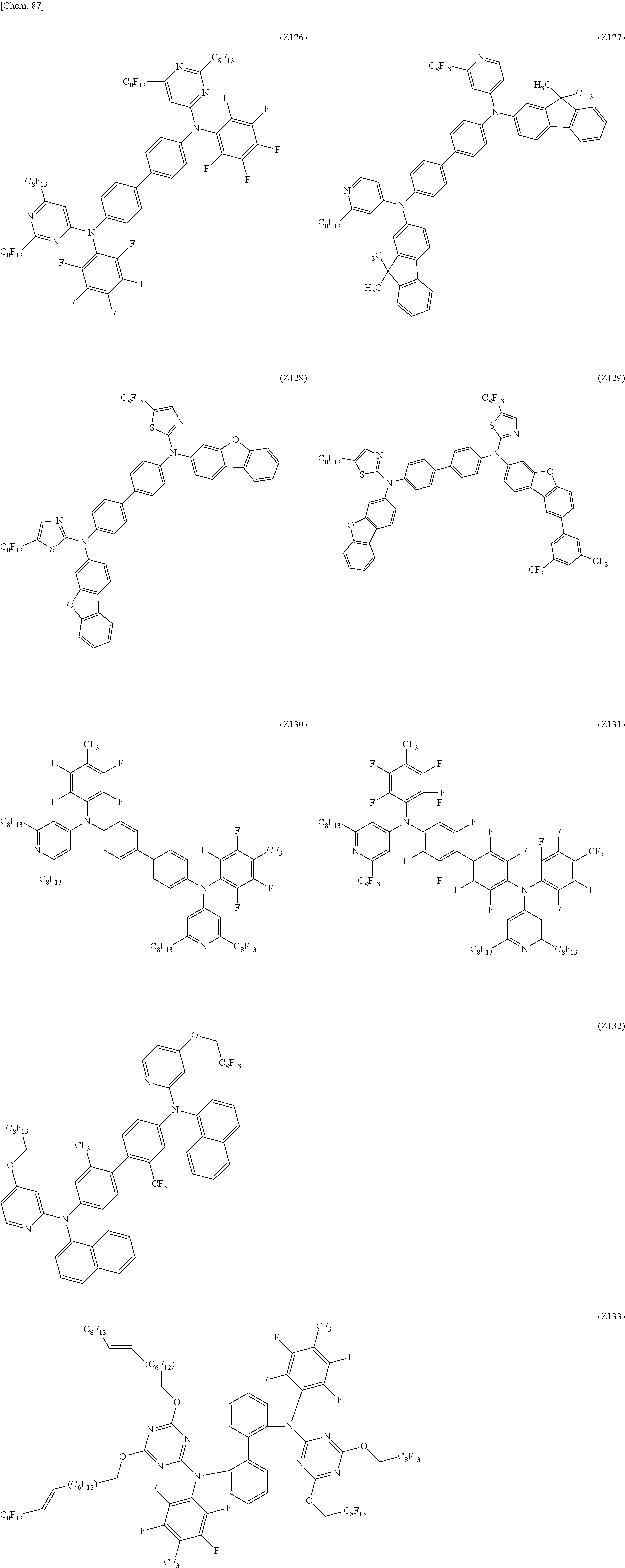

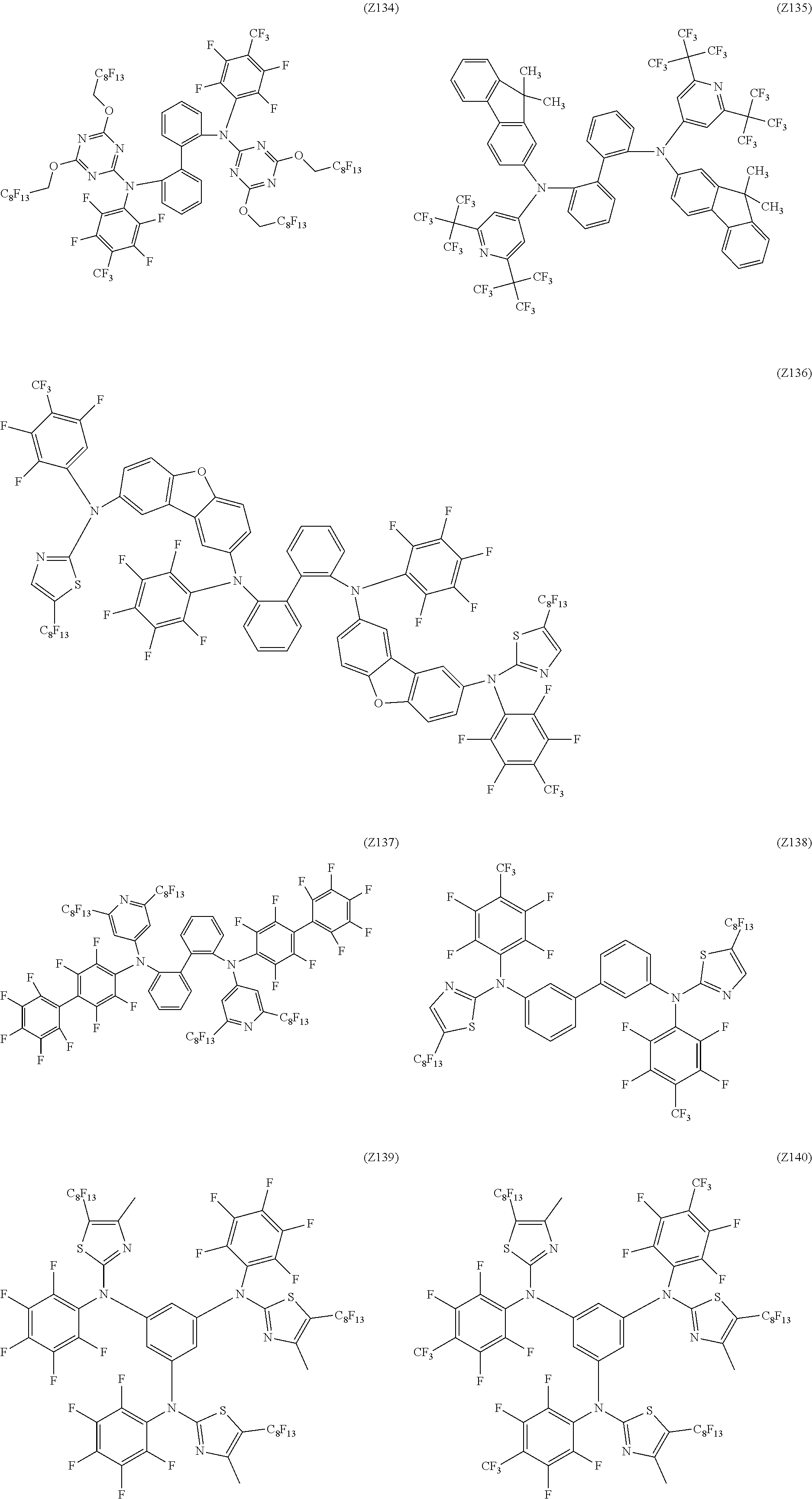

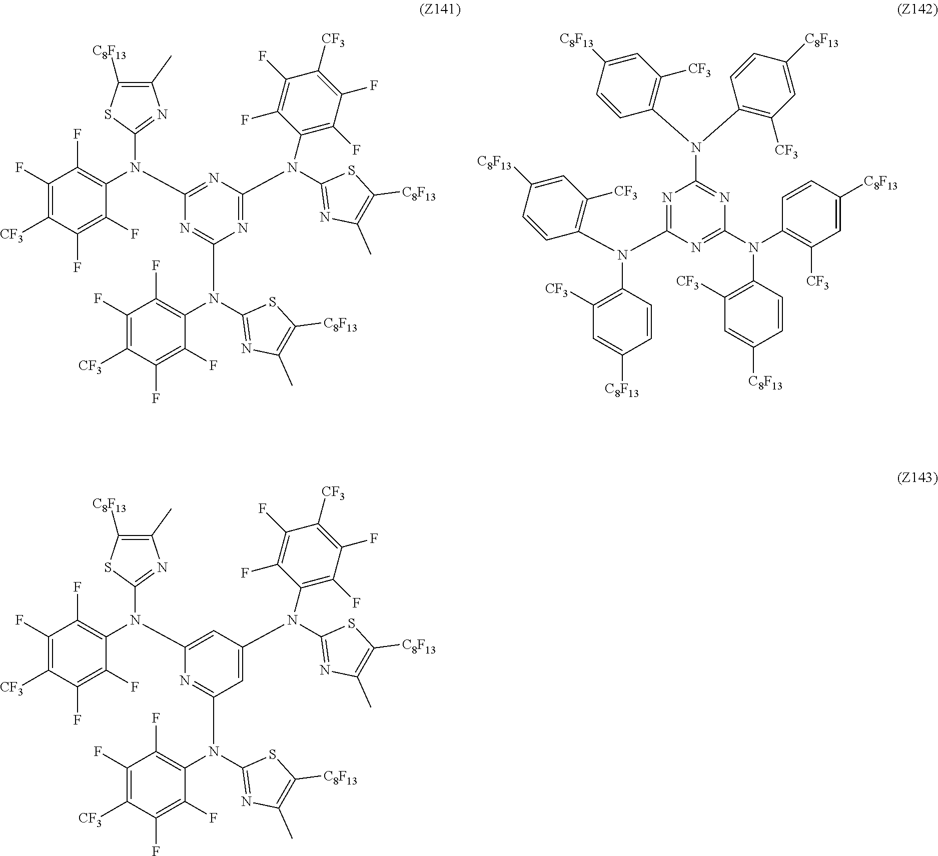

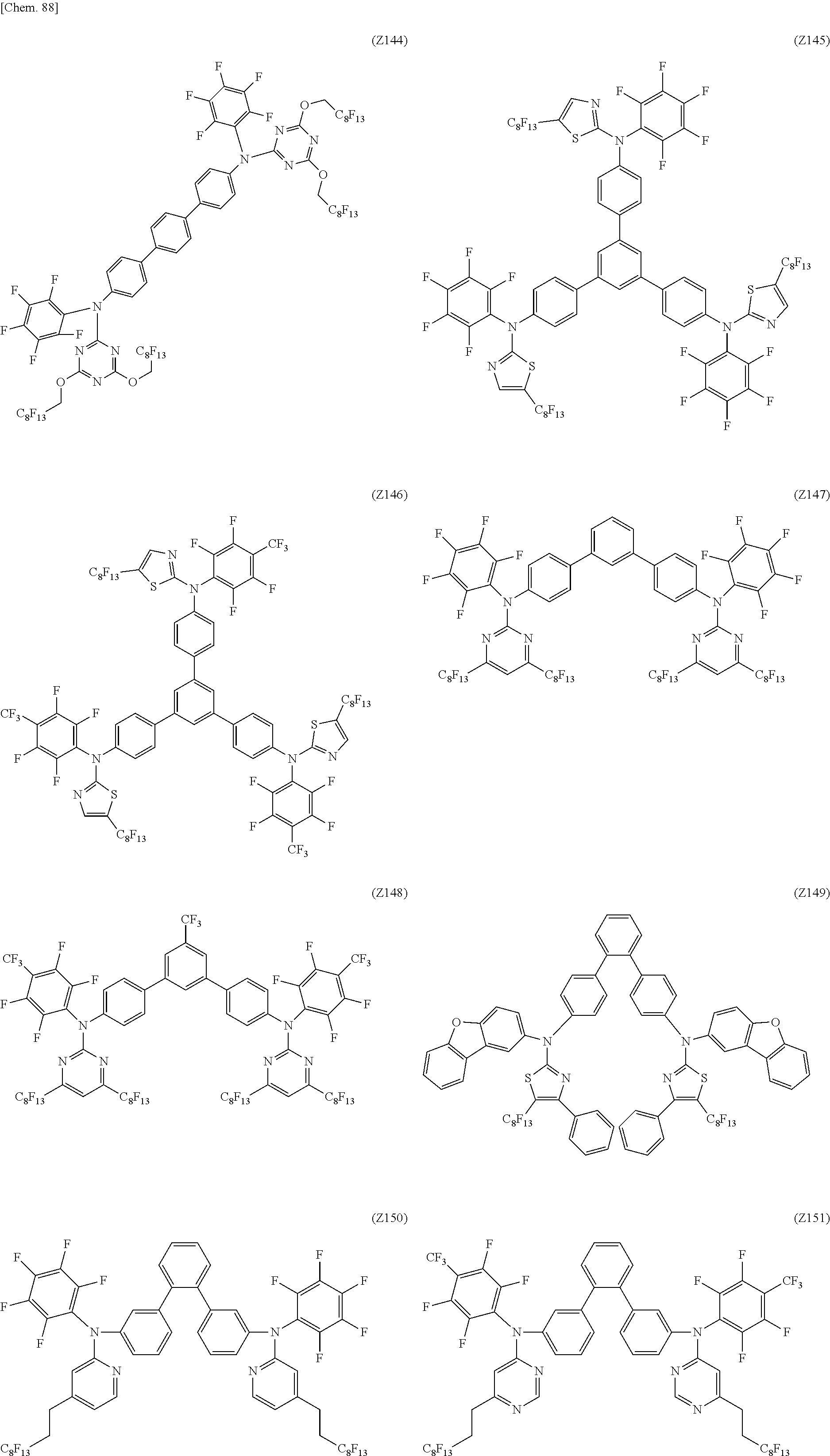

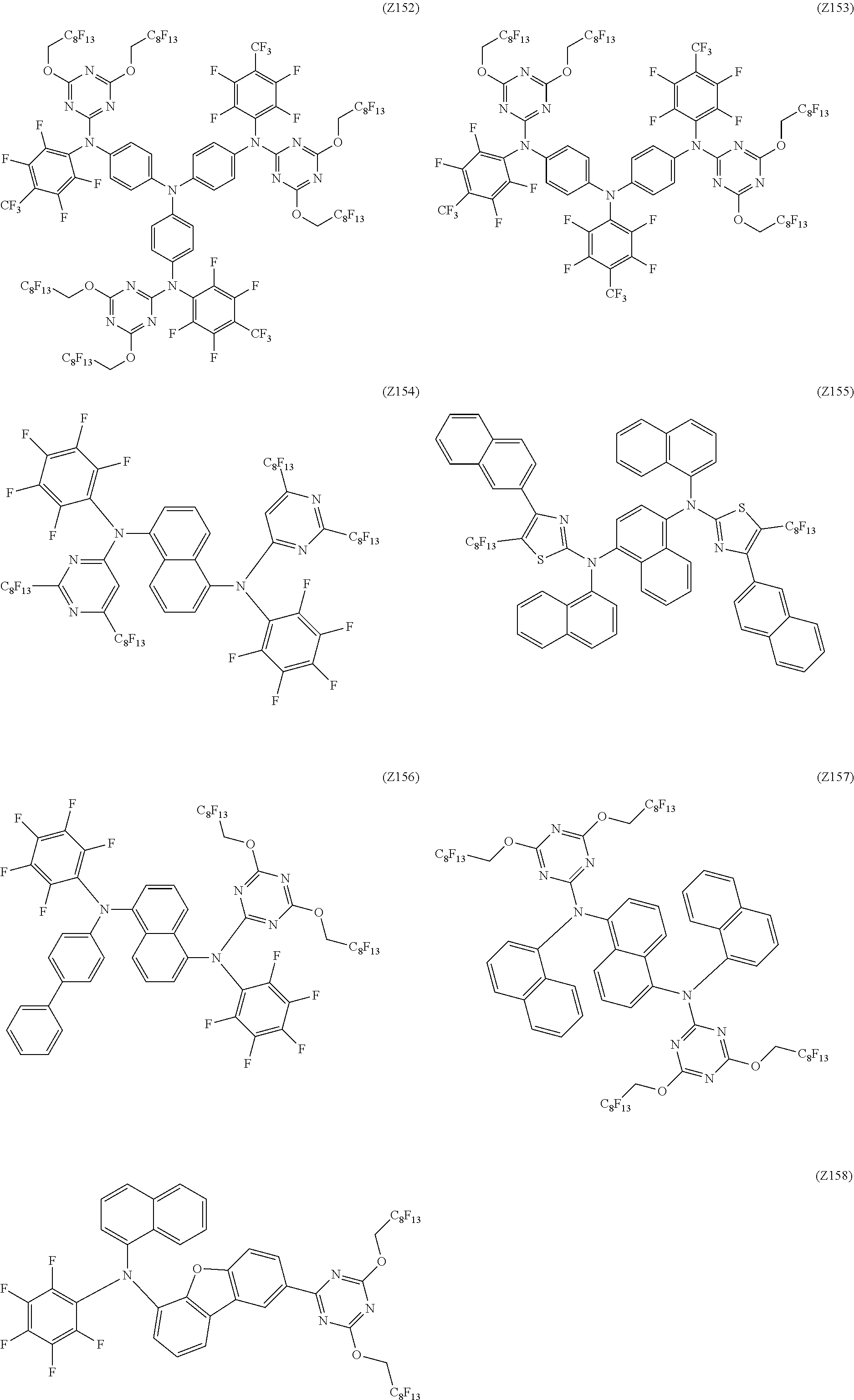

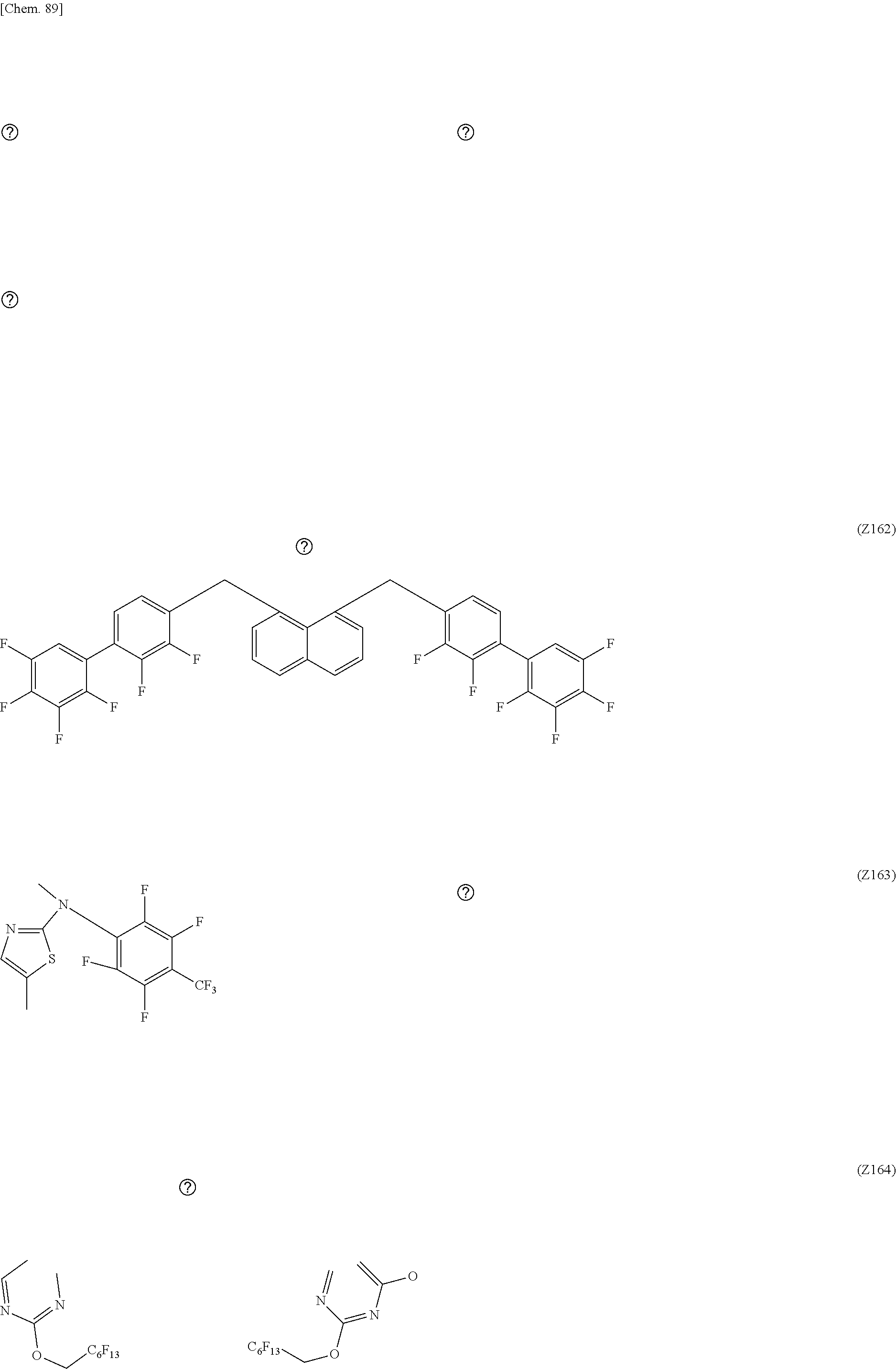

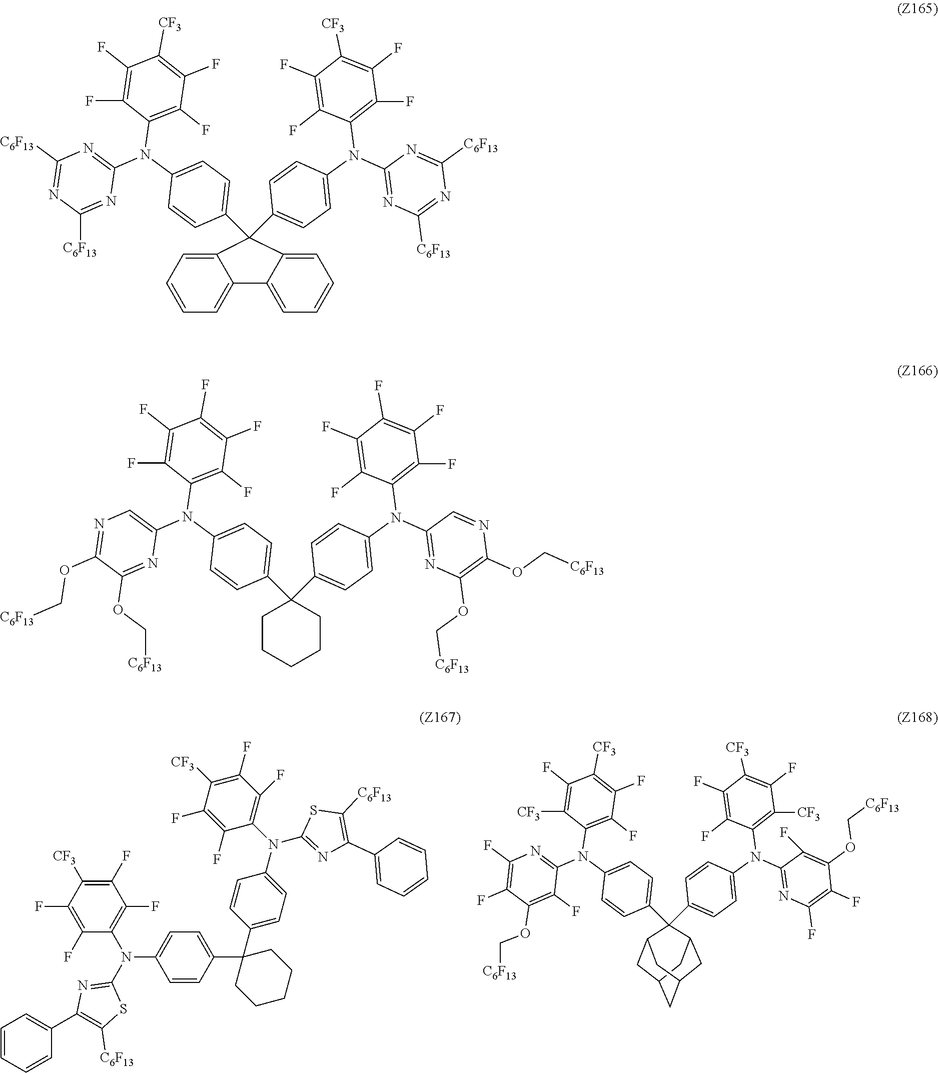

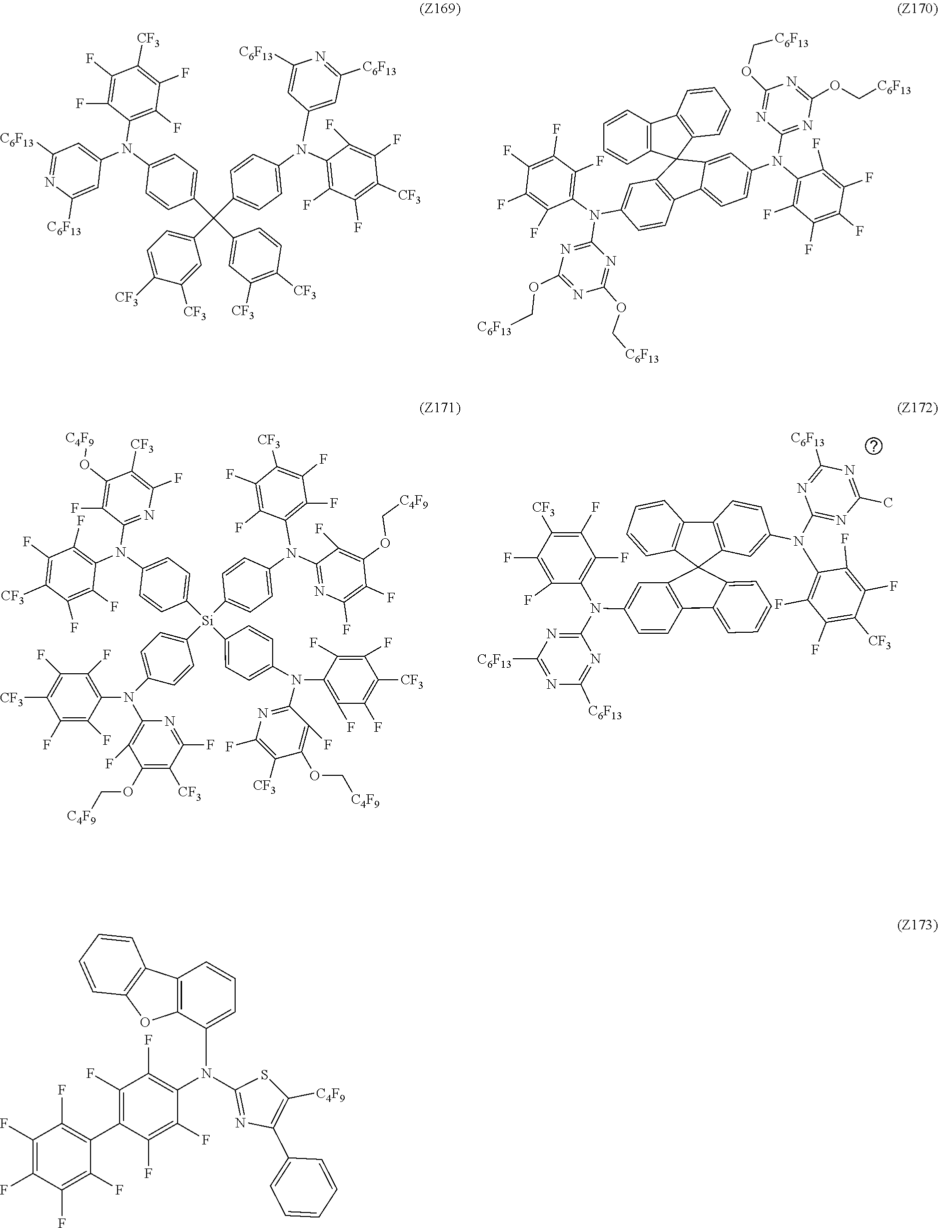

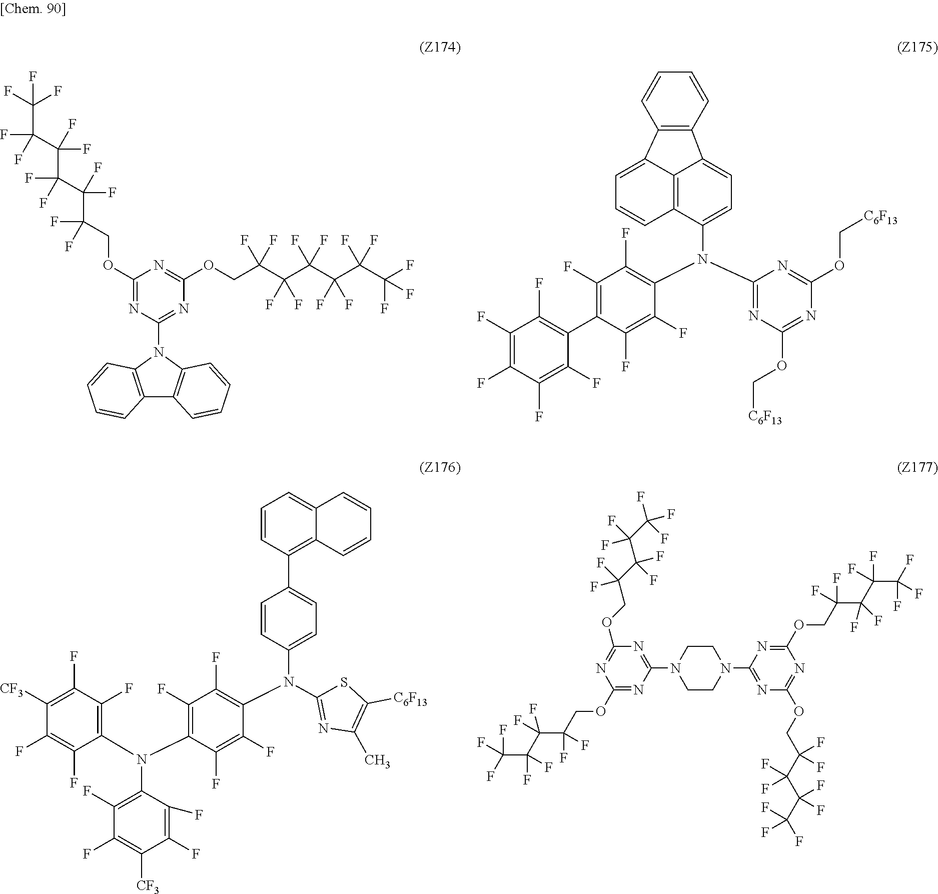

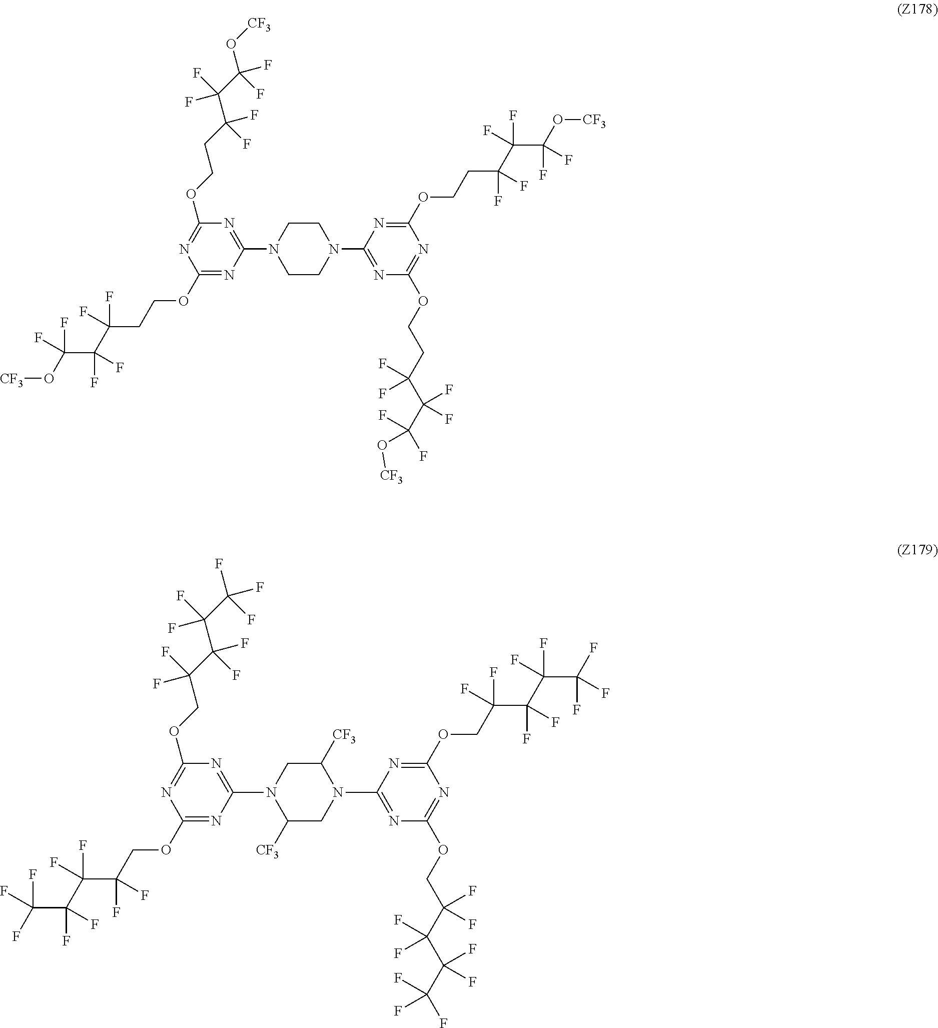 [SMILES: FC(F)(F)OC(F)(F)C(F)(F)C(F)(F)CCOc1nc(OCCC(F)(F)C(F)(F)C(F)(F)OC(F)(F)F)nc(N2CCN(c3nc(OCCC(F)(F)C(F)(F)C(F)(F)OC(F)(F)F)nc(OCCC(F)(F)C(F)(F)C(F)(F)OC(F)(F)F)n3)CC2)n1 ; FC(F)(F)C1CN(c2nc(OCC(F)(F)C(F)(F)C(F)(F)C(F)(F)F)nc(OCC(F)(F)C(F)(F)C(F)(F)C(F)(F)F)n2)C(C(F)(F)F)CN1c1nc(OCC(F)(F)C(F)(F)C(F)(F)C(F)(F)F)nc(OCC(F)(F)C(F)(F)C(F)(F)C(F)(F)F)n1]

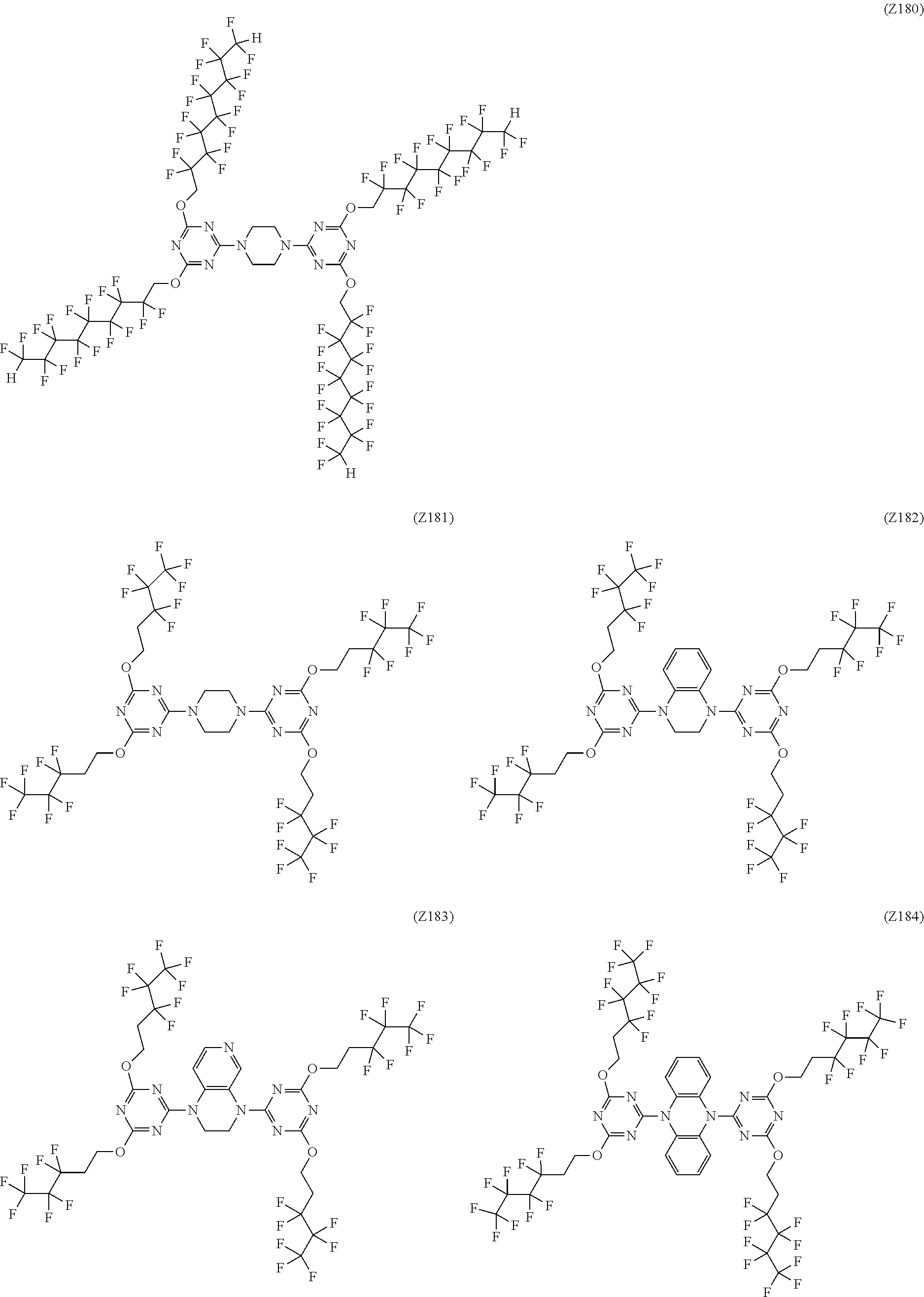

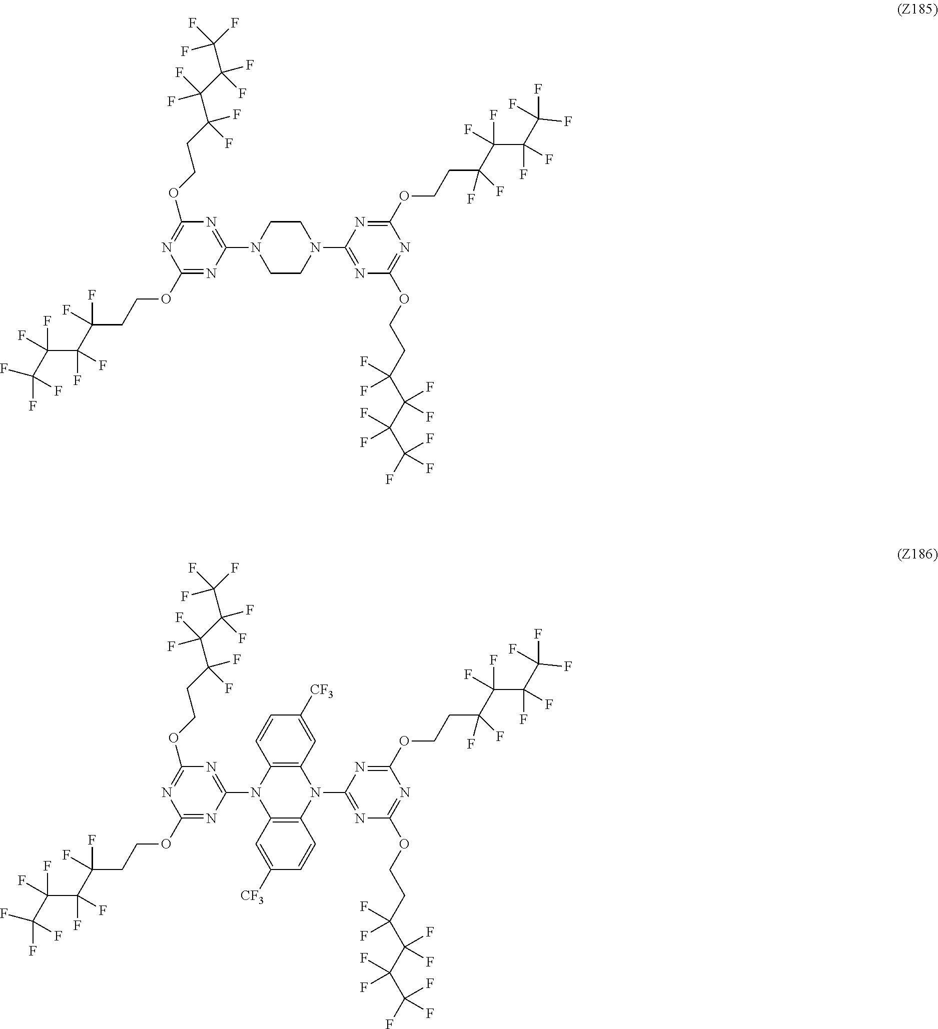 [SMILES: FC(F)(F)C(F)(F)C(F)(F)C(F)(F)CCOc1nc(OCCC(F)(F)C(F)(F)C(F)(F)C(F)(F)F)nc(N2CCN(c3nc(OCCC(F)(F)C(F)(F)C(F)(F)C(F)(F)F)nc(OCCC(F)(F)C(F)(F)C(F)(F)C(F)(F)F)n3)CC2)n1 ; FC(F)(F)c1ccc2c(c1)N(c1nc(OCCC(F)(F)C(F)(F)C(F)(F)C(F)(F)F)nc(OCCC(F)(F)C(F)(F)C(F)(F)C(F)(F)F)n1)c1ccc(C(F)(F)F)cc1N2c1nc(OCCC(F)(F)C(F)(F)C(F)(F)C(F)(F)F)nc(OCCC(F)(F)C(F)(F)C(F)(F)C(F)(F)F)n1]

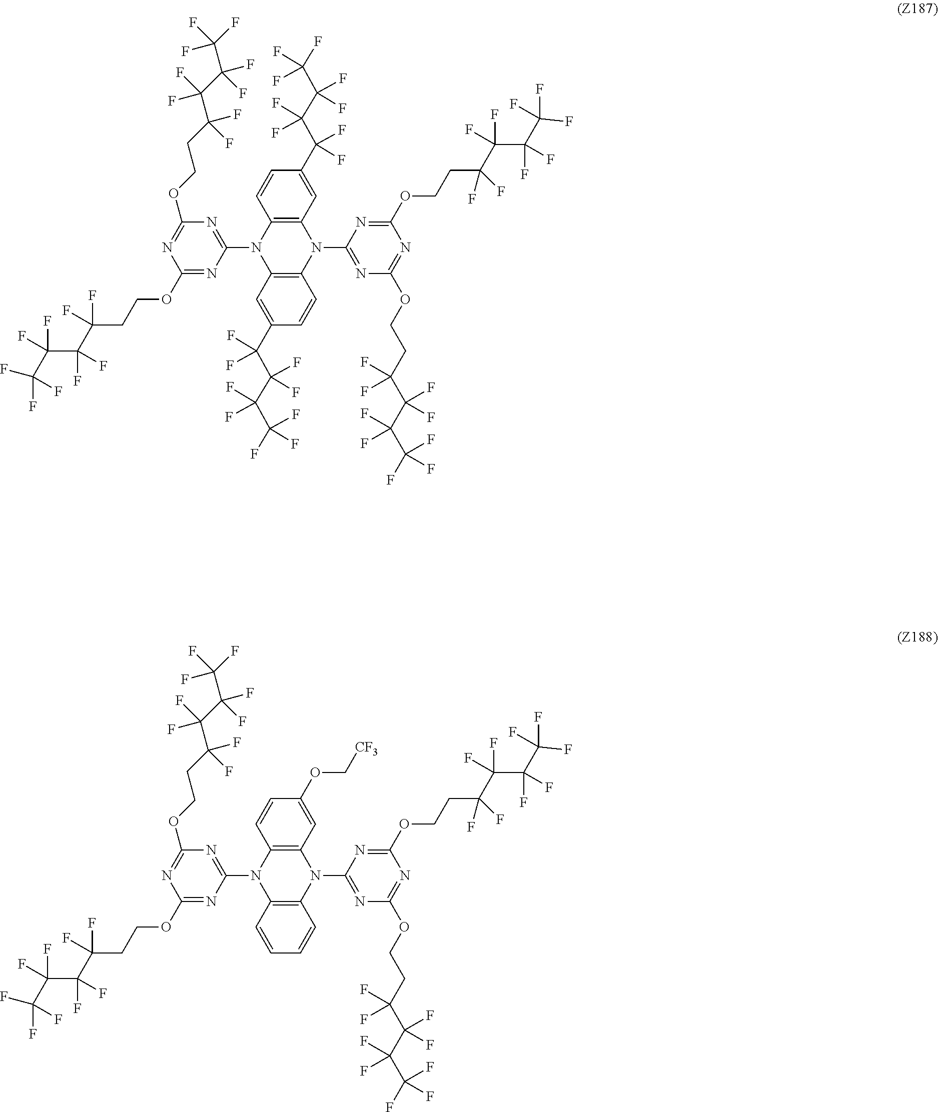 [SMILES: FC(F)(F)C(F)(F)C(F)(F)C(F)(F)CCOc1nc(OCCC(F)(F)C(F)(F)C(F)(F)C(F)(F)F)nc(N2c3ccc(C(F)(F)C(F)(F)C(F)(F)C(F)(F)F)cc3N(c3nc(OCCC(F)(F)C(F)(F)C(F)(F)C(F)(F)F)nc(OCCC(F)(F)C(F)(F)C(F)(F)C(F)(F)F)n3)c3ccc(C(F)(F)C(F)(F)C(F)(F)C(F)(F)F)cc32)n1 ; FC(F)(F)COc1ccc2c(c1)N(c1nc(OCCC(F)(F)C(F)(F)C(F)(F)C(F)(F)F)nc(OCCC(F)(F)C(F)(F)C(F)(F)C(F)(F)F)n1)c1ccccc1N2c1nc(OCCC(F)(F)C(F)(F)C(F)(F)C(F)(F)F)nc(OCCC(F)(F)C(F)(F)C(F)(F)C(F)(F)F)n1]

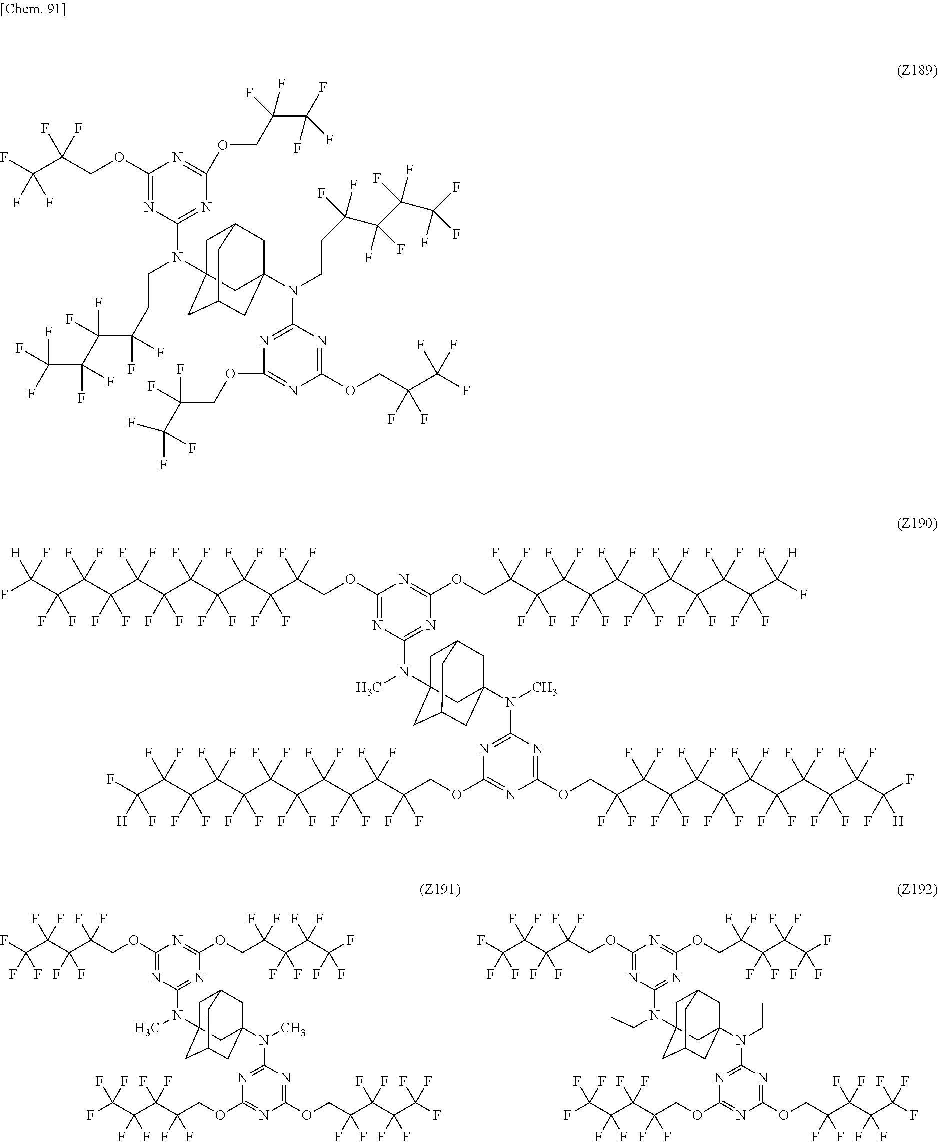

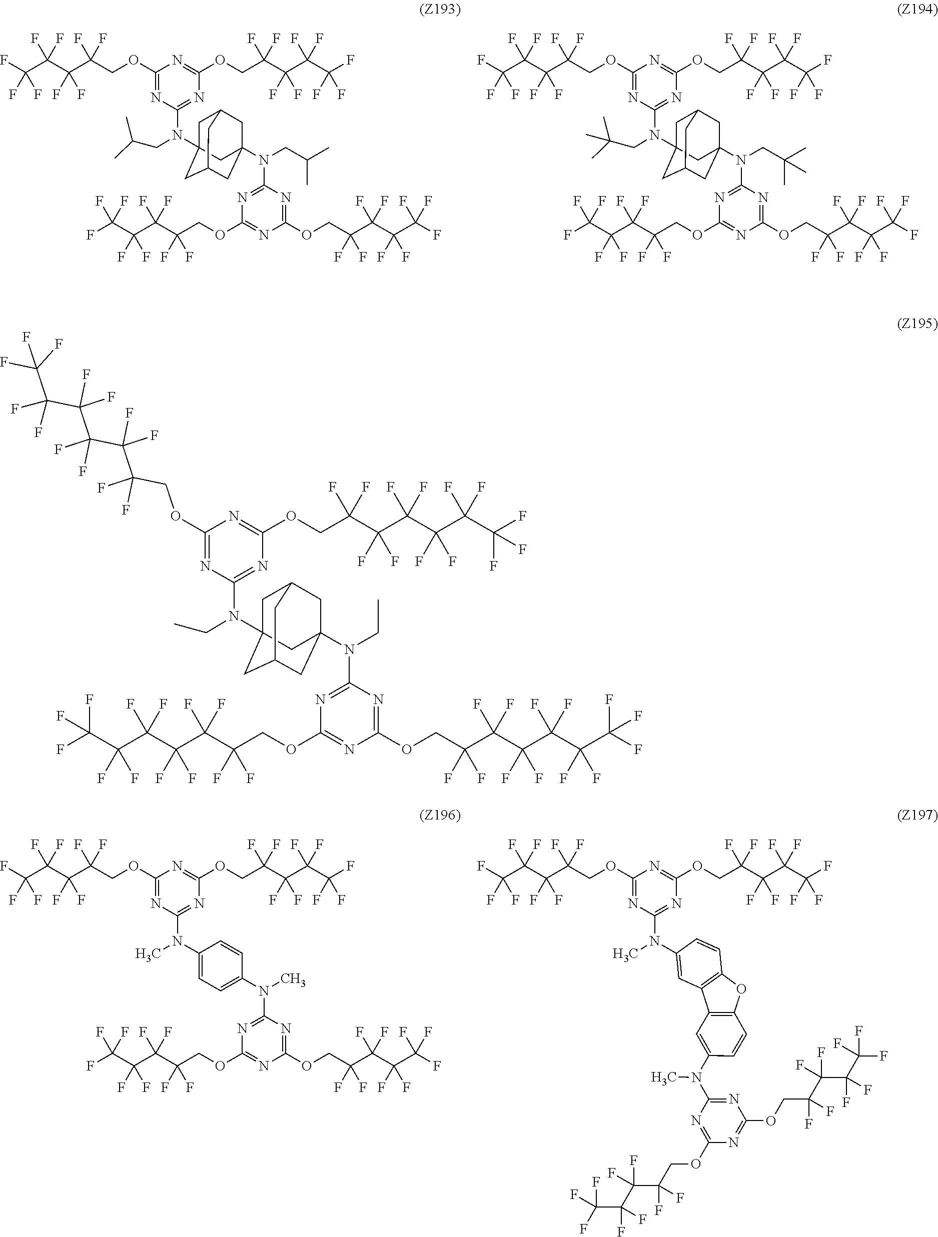

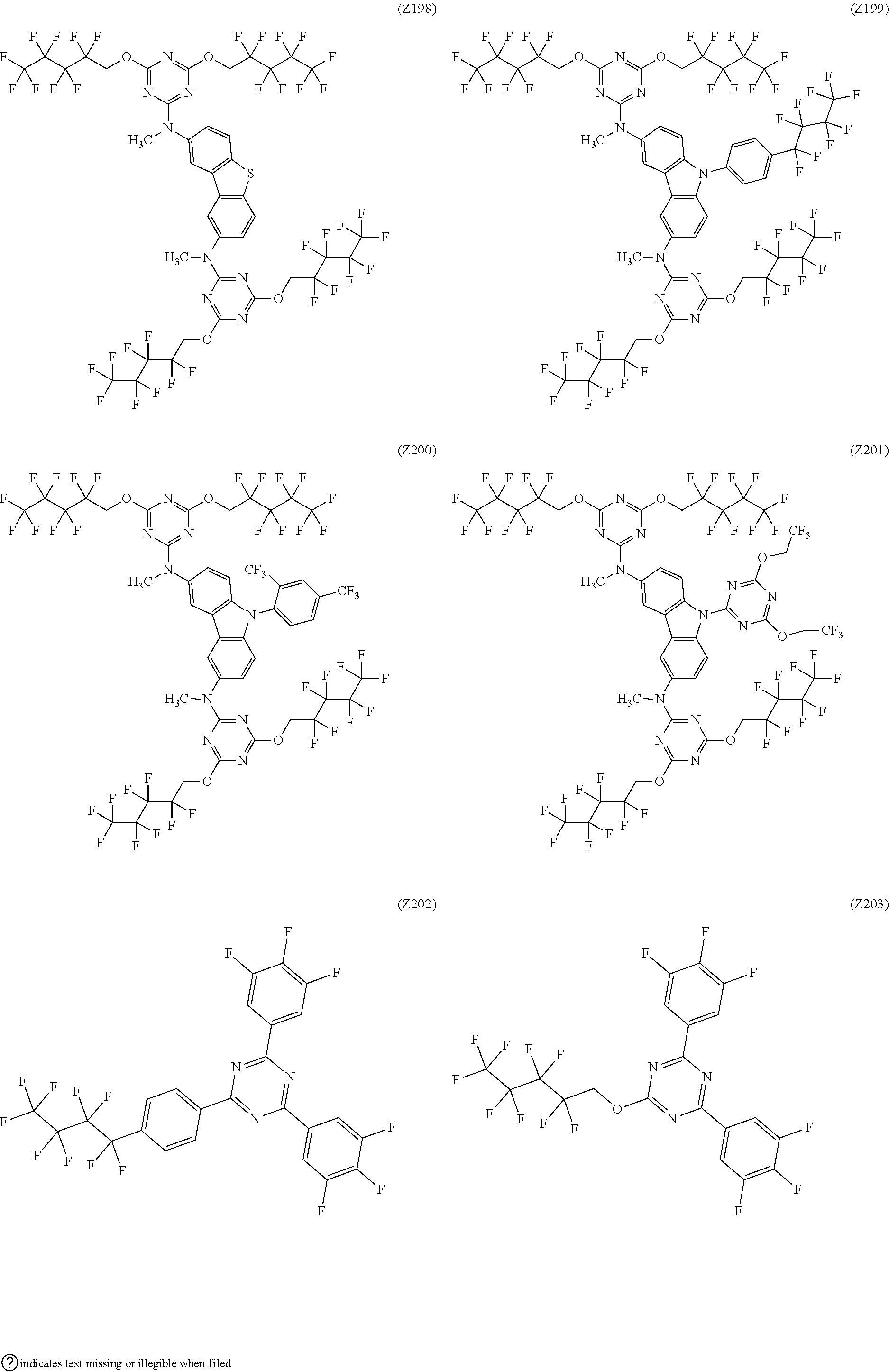

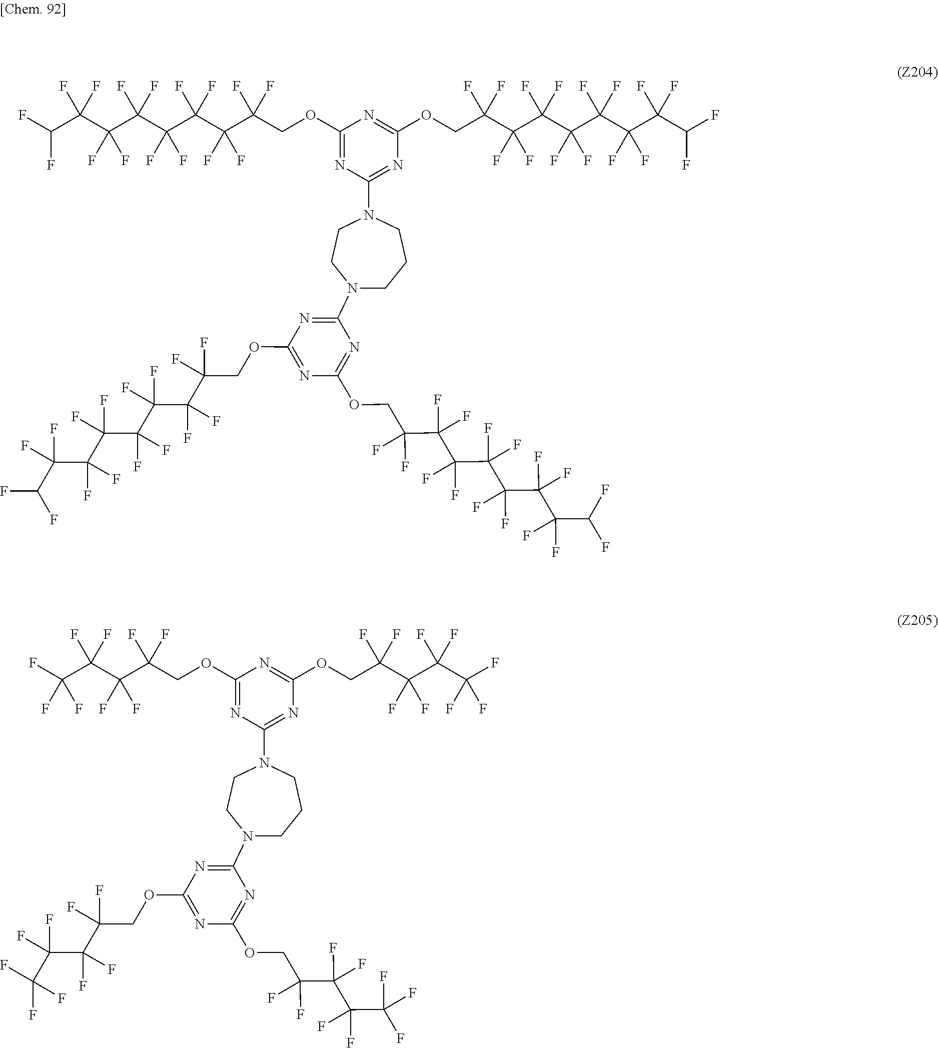 [SMILES: FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(N2CCCN(c3nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n3)CC2)n1 ; FC(F)(F)C(F)(F)C(F)(F)C(F)(F)COc1nc(OCC(F)(F)C(F)(F)C(F)(F)C(F)(F)F)nc(N2CCCN(c3nc(OCC(F)(F)C(F)(F)C(F)(F)C(F)(F)F)nc(OCC(F)(F)C(F)(F)C(F)(F)C(F)(F)F)n3)CC2)n1]

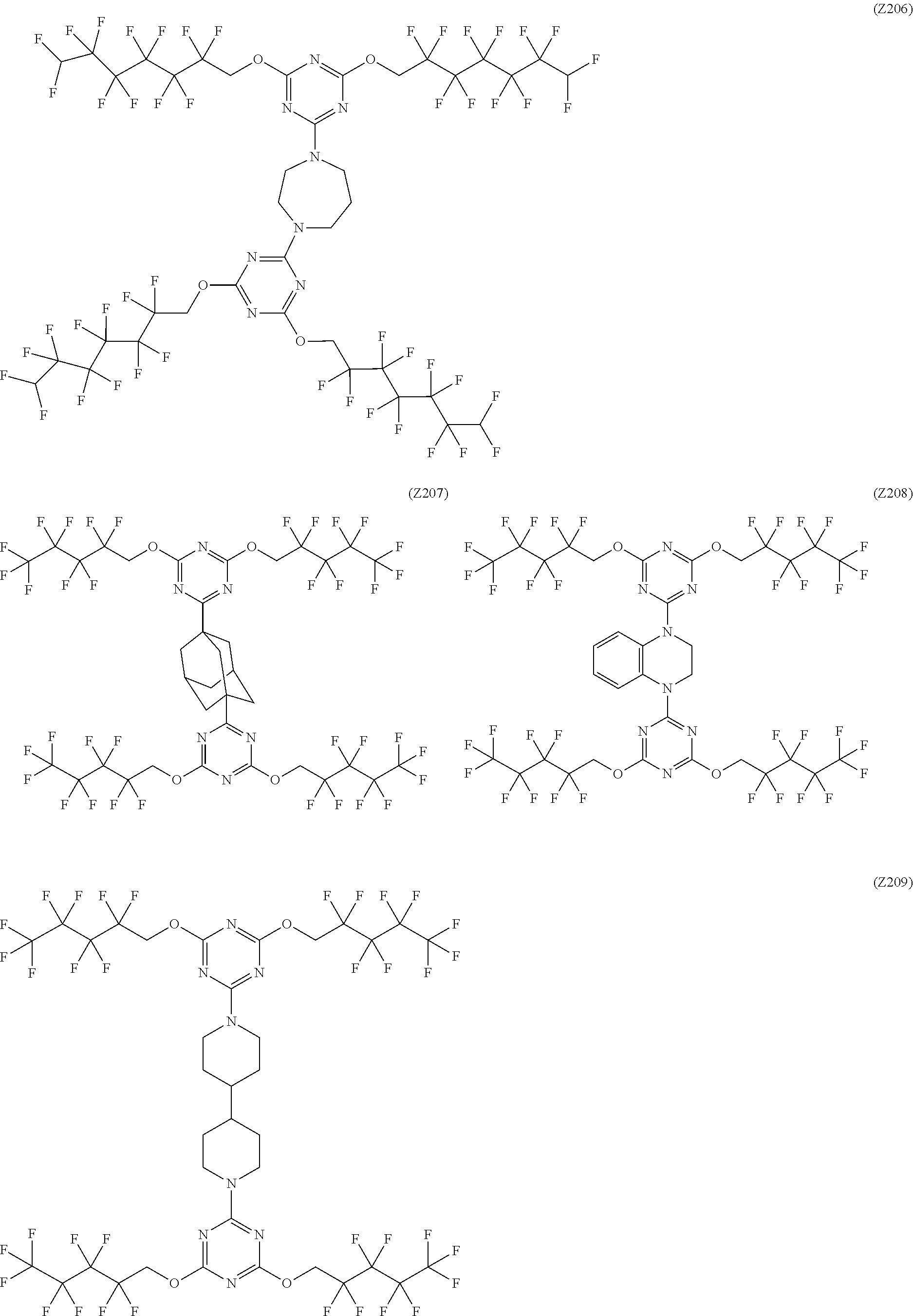

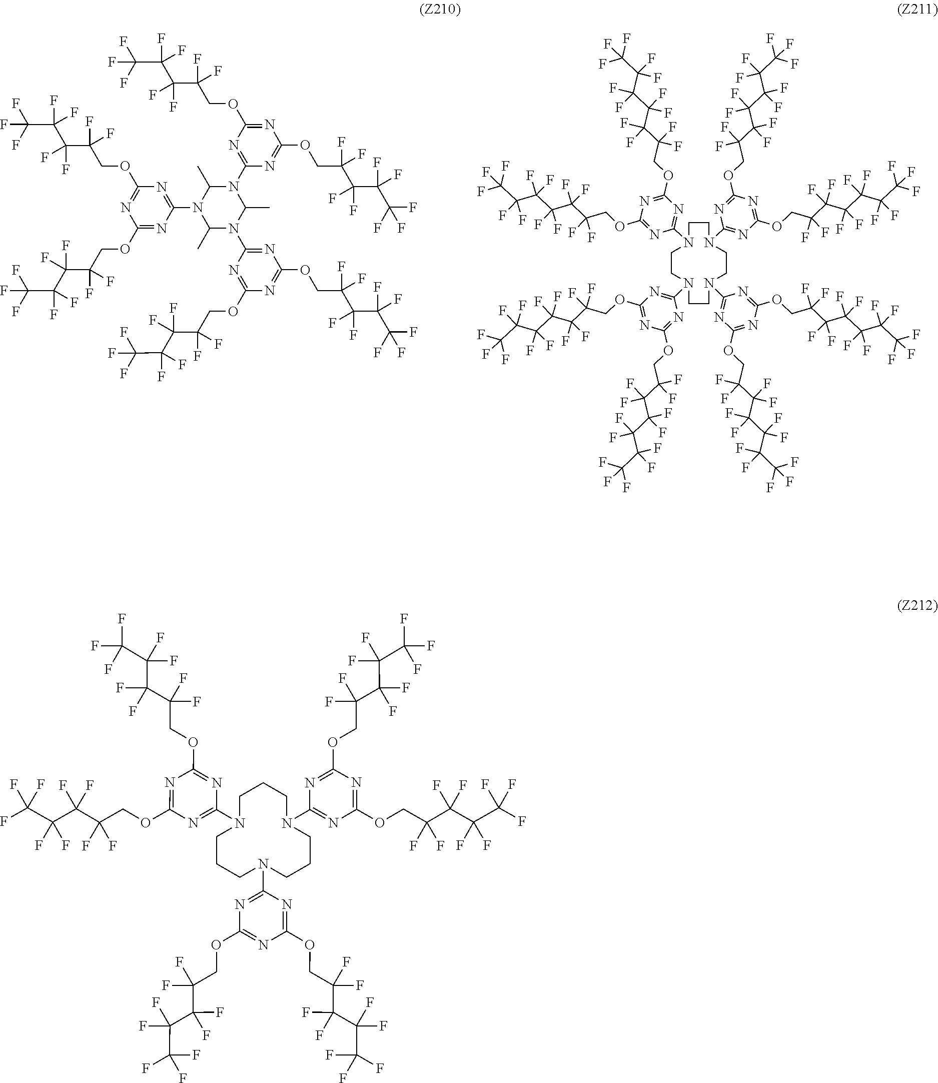

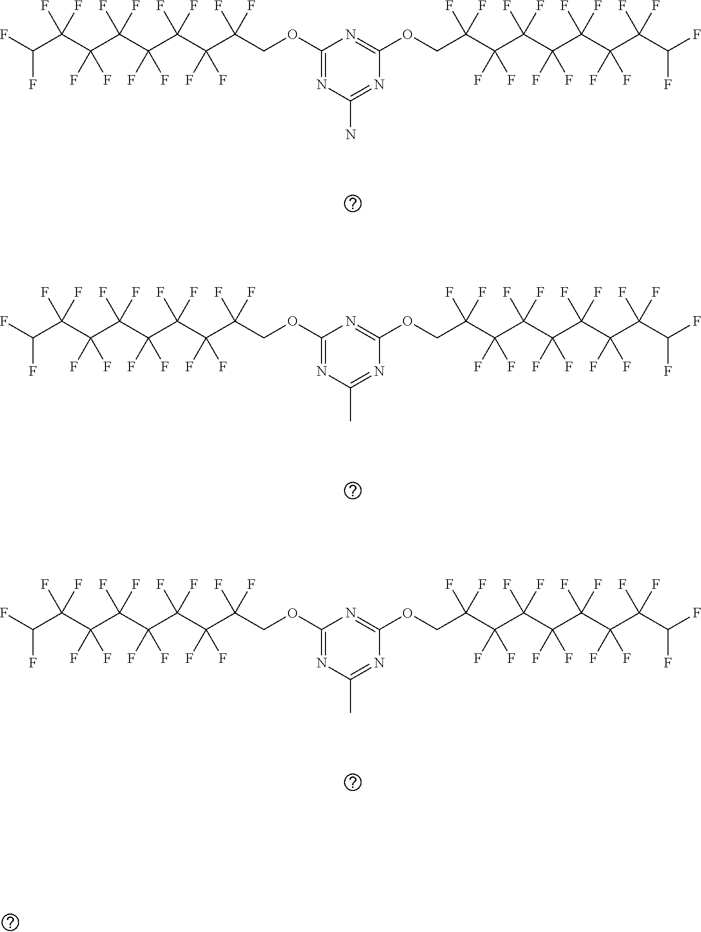 [SMILES: Nc1nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n1 ; Cc1nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n1 ; Cc1nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n1]

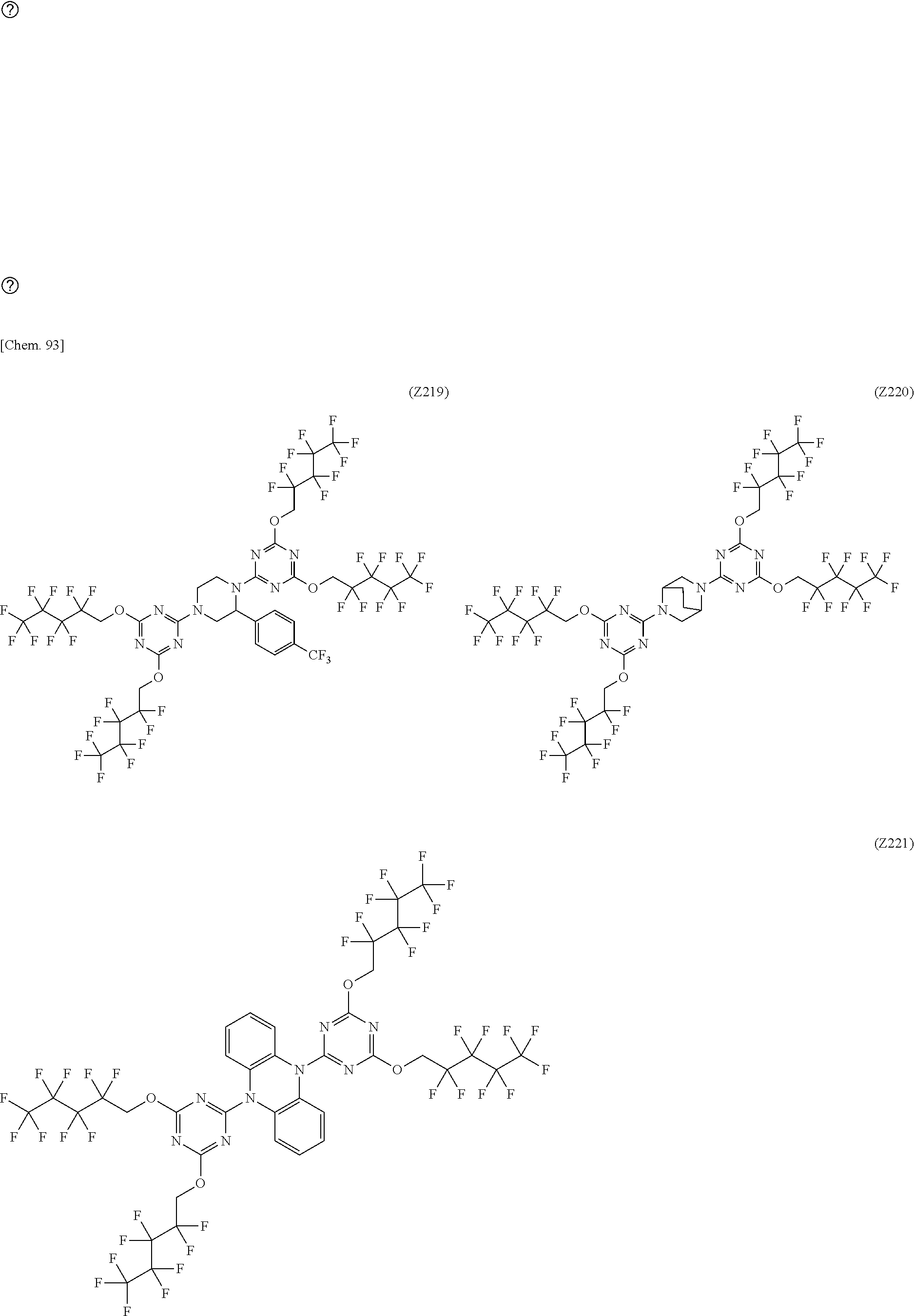

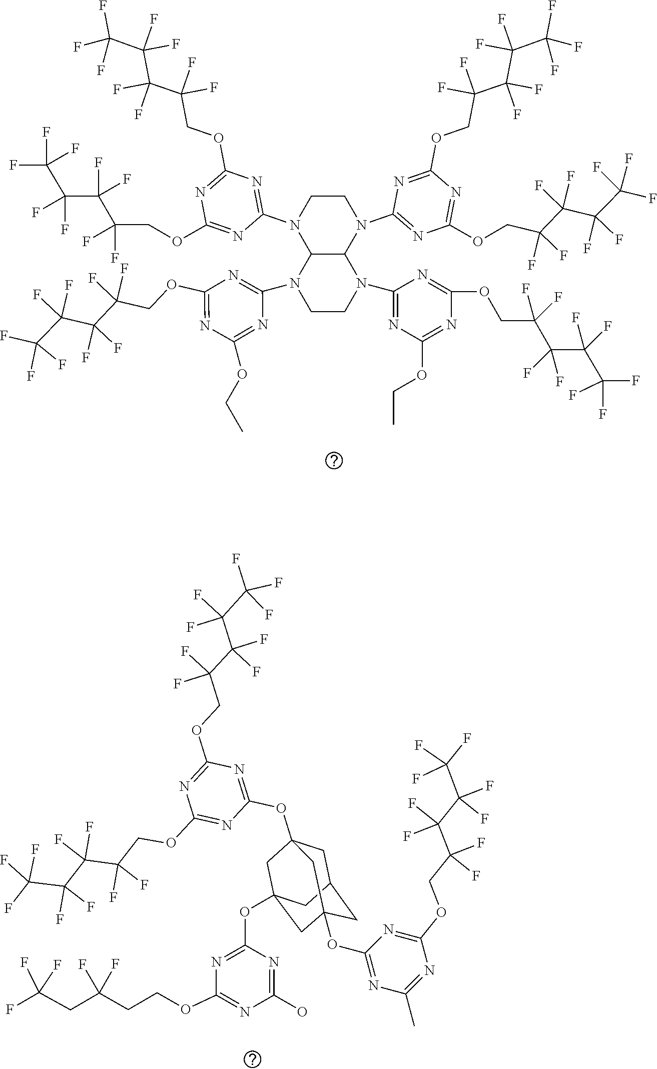 [SMILES: CCOc1nc(OCC(F)(F)C(F)(F)C(F)(F)C(F)(F)F)nc(N2CCN(c3nc(OCC)nc(OCC(F)(F)C(F)(F)C(F)(F)C(F)(F)F)n3)C3C2N(c2nc(OCC(F)(F)C(F)(F)C(F)(F)C(F)(F)F)nc(OCC(F)(F)C(F)(F)C(F)(F)C(F)(F)F)n2)CCN3c2nc(OCC(F)(F)C(F)(F)C(F)(F)C(F)(F)F)nc(OCC(F)(F)C(F)(F)C(F)(F)C(F)(F)F)n2)n1 ; Cc1nc(OCC(F)(F)C(F)(F)C(F)(F)C(F)(F)F)nc(OC23CC4CC(Oc5nc(O)nc(OCCC(F)(F)CC(F)(F)F)n5)(C2)CC(Oc2nc(OCC(F)(F)C(F)(F)C(F)(F)C(F)(F)F)nc(OCC(F)(F)C(F)(F)C(F)(F)C(F)(F)F)n2)(C4)C3)n1]

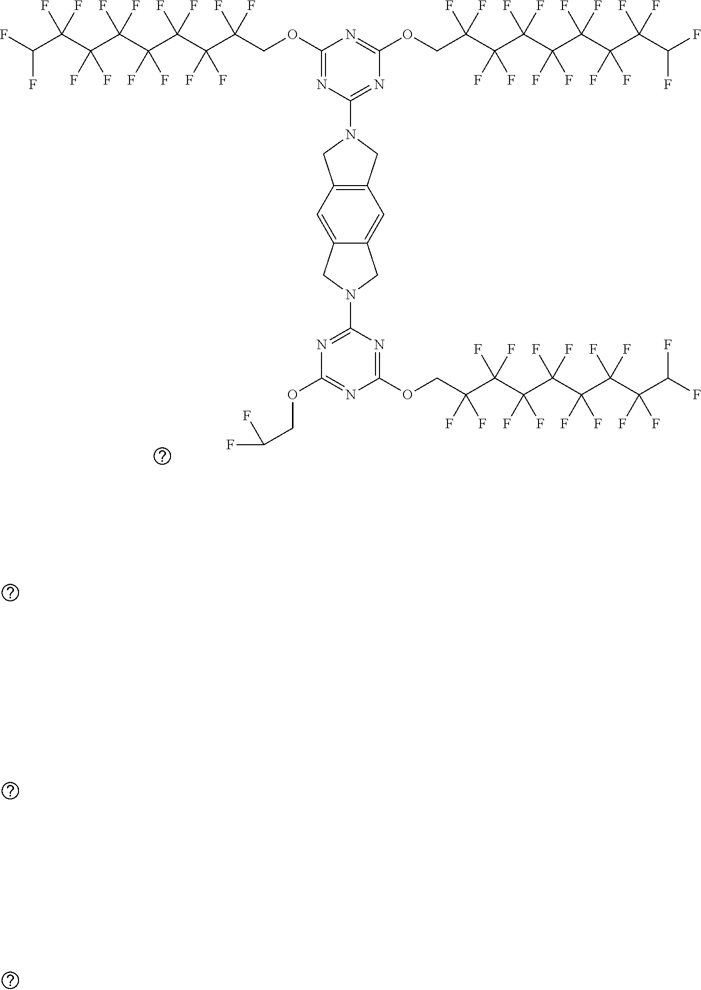 [SMILES: FC(F)COc1nc(OCC(F)(F)C(F)(F)C(F)(F)C(F)(F)C(F)(F)C(F)(F)C(F)(F)C(F)F)nc(N2Cc3cc4c(cc3C2)CN(c2nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n2)C4)n1]

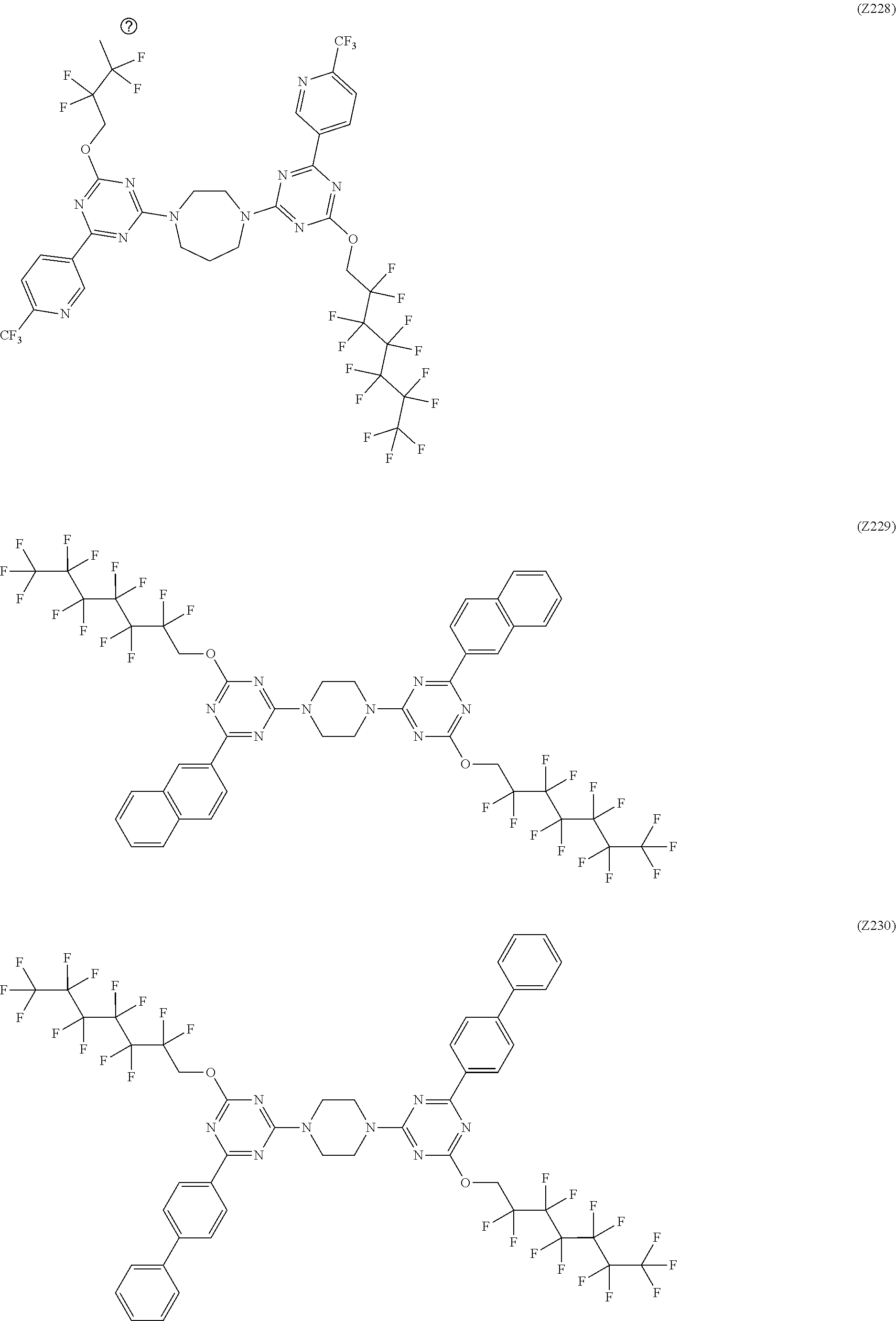 [SMILES: CC(F)(F)C(F)(F)COc1nc(-c2ccc(C(F)(F)F)nc2)nc(N2CCCN(c3nc(OCC(F)(F)C(F)(F)C(F)(F)C(F)(F)C(F)(F)C(F)(F)F)nc(-c4ccc(C(F)(F)F)nc4)n3)CC2)n1 ; FC(F)(F)C(F)(F)C(F)(F)C(F)(F)C(F)(F)C(F)(F)COc1nc(-c2ccc3ccccc3c2)nc(N2CCN(c3nc(OCC(F)(F)C(F)(F)C(F)(F)C(F)(F)C(F)(F)C(F)(F)F)nc(-c4ccc5ccccc5c4)n3)CC2)n1 ; FC(F)(F)C(F)(F)C(F)(F)C(F)(F)C(F)(F)C(F)(F)COc1nc(-c2ccc(-c3ccccc3)cc2)nc(N2CCN(c3nc(OCC(F)(F)C(F)(F)C(F)(F)C(F)(F)C(F)(F)C(F)(F)F)nc(-c4ccc(-c5ccccc5)cc4)n3)CC2)n1]

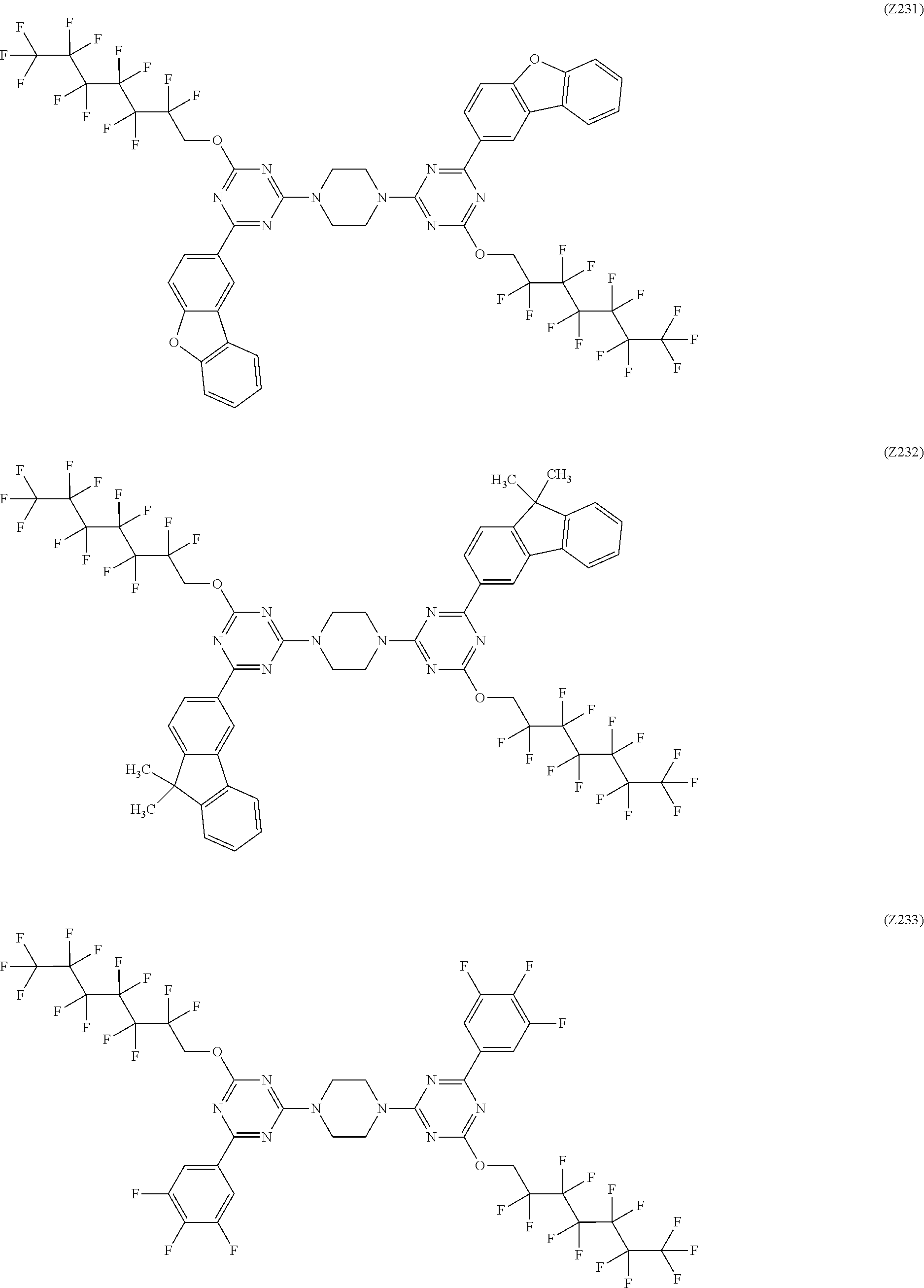

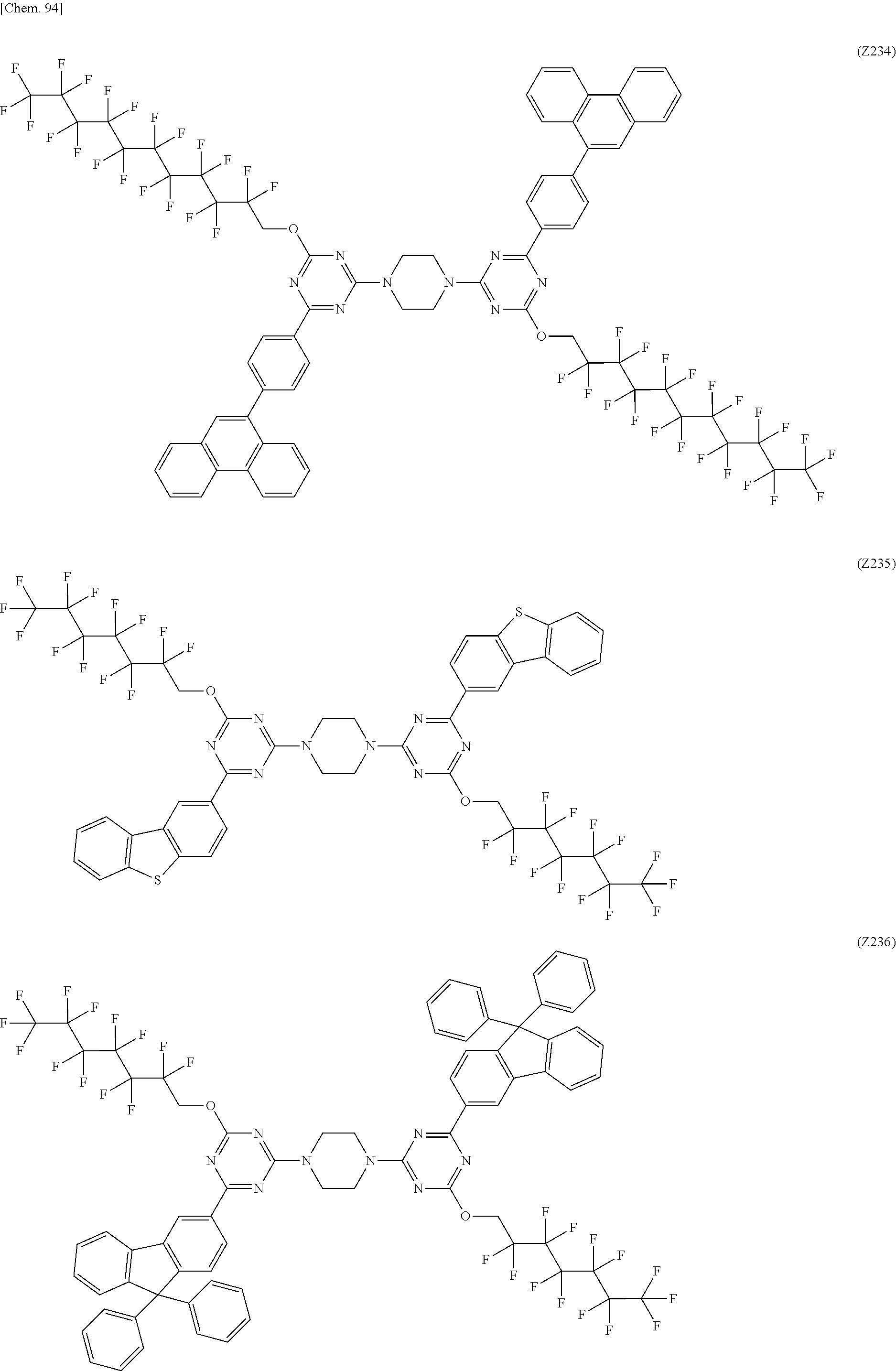

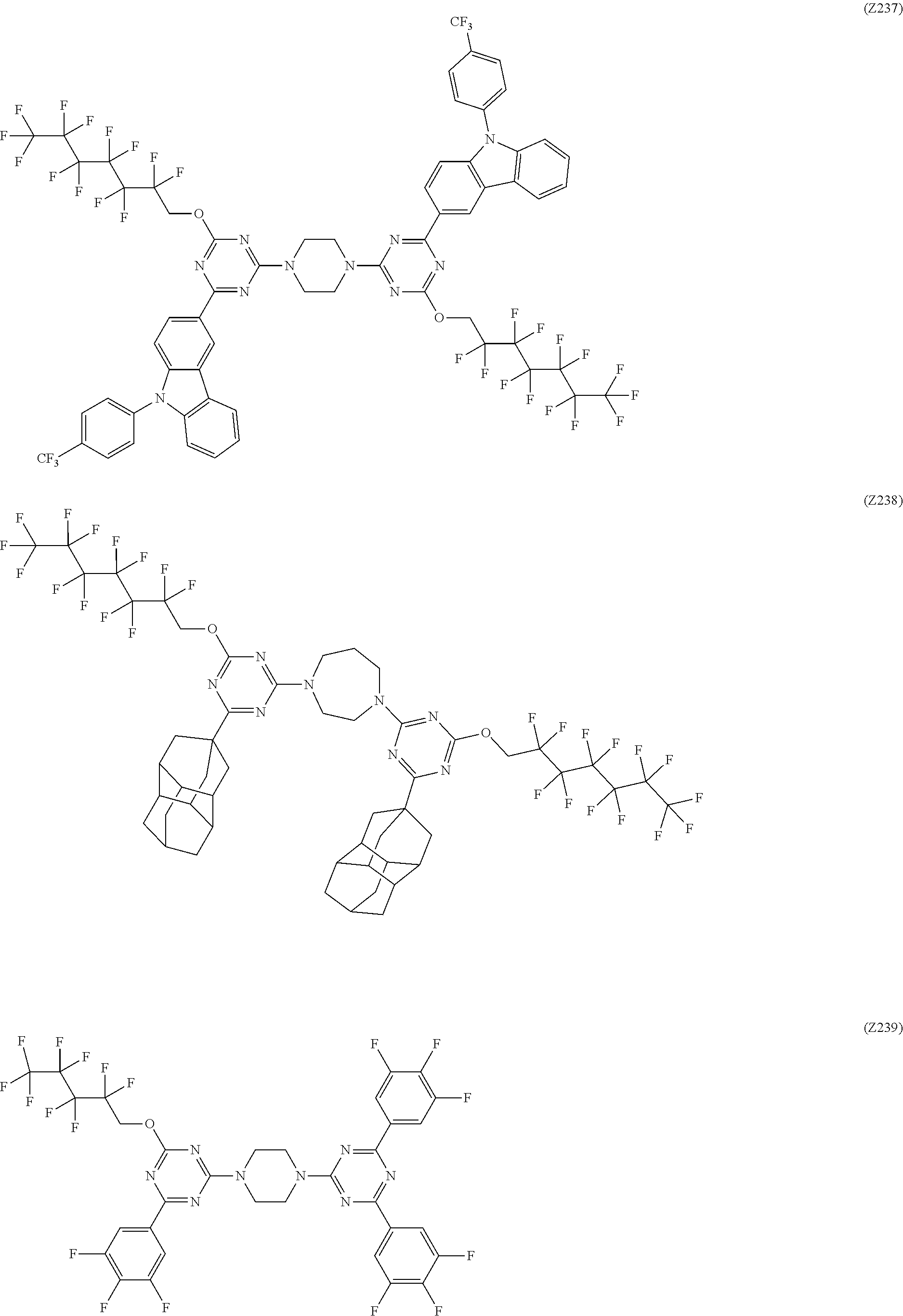

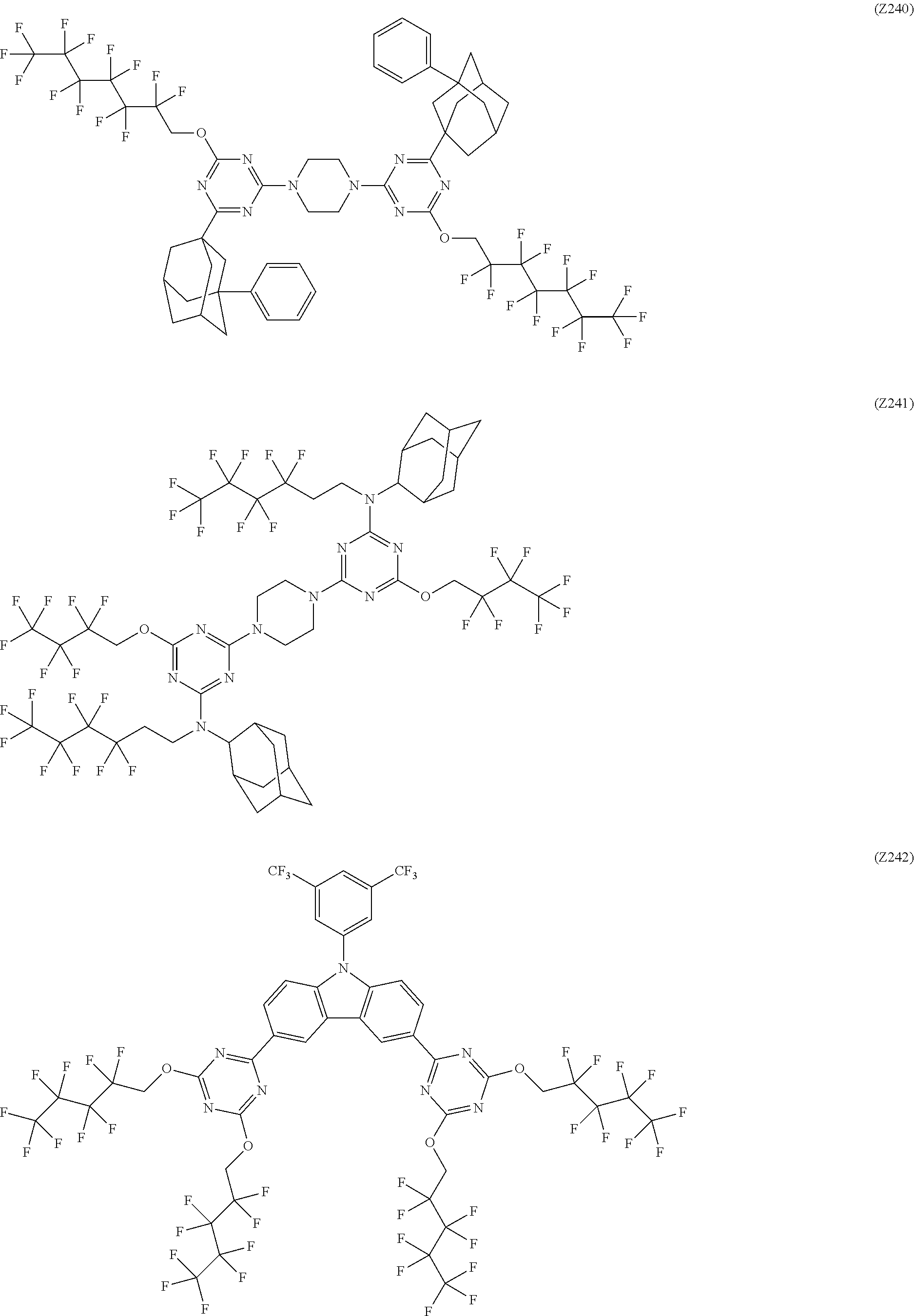

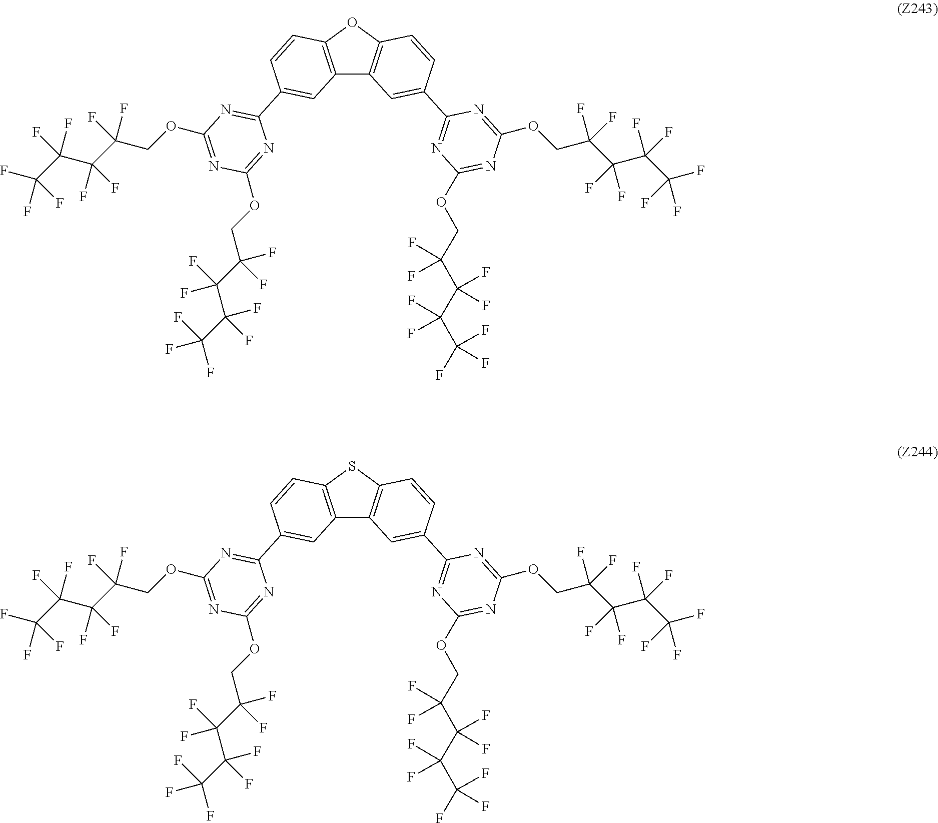 [SMILES: FC(F)(F)C(F)(F)C(F)(F)C(F)(F)COc1nc(OCC(F)(F)C(F)(F)C(F)(F)C(F)(F)F)nc(-c2ccc3oc4ccc(-c5nc(OCC(F)(F)C(F)(F)C(F)(F)C(F)(F)F)nc(OCC(F)(F)C(F)(F)C(F)(F)C(F)(F)F)n5)cc4c3c2)n1 ; FC(F)(F)C(F)(F)C(F)(F)C(F)(F)COc1nc(OCC(F)(F)C(F)(F)C(F)(F)C(F)(F)F)nc(-c2ccc3sc4ccc(-c5nc(OCC(F)(F)C(F)(F)C(F)(F)C(F)(F)F)nc(OCC(F)(F)C(F)(F)C(F)(F)C(F)(F)F)n5)cc4c3c2)n1]

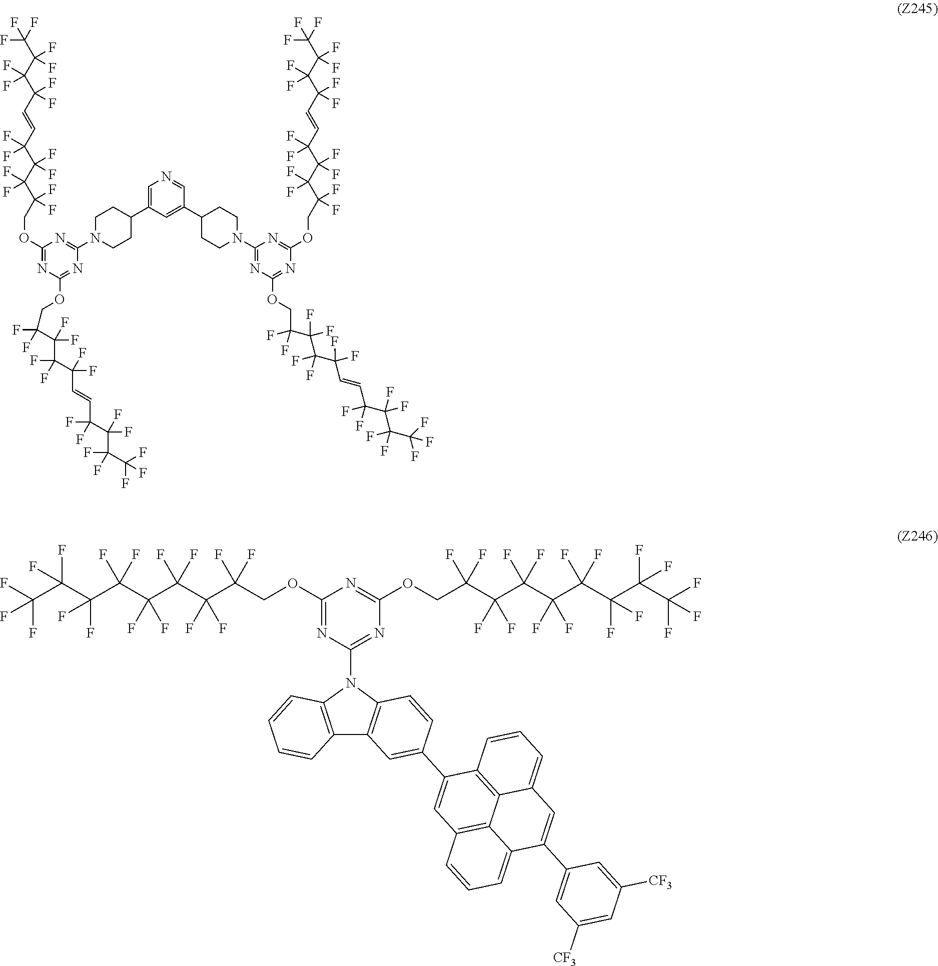

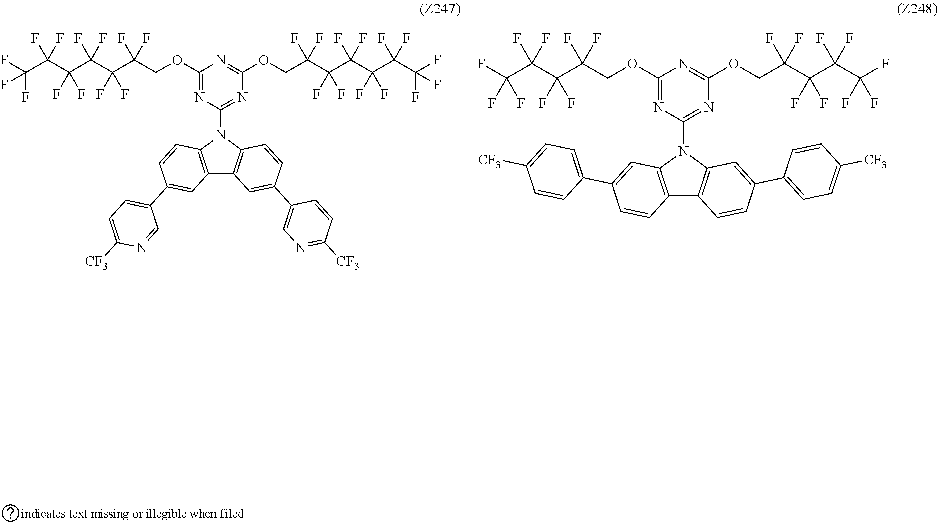 [SMILES: FC(F)(F)c1ccc(-c2ccc3c(c2)c2cc(-c4ccc(C(F)(F)F)nc4)ccc2n3-c2nc(OCC(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)F)n2)cn1 ; FC(F)(F)c1ccc(-c2ccc3c4ccc(-c5ccc(C(F)(F)F)cc5)cc4n(-c4nc(OCC(F)(F)C(F)(F)C(F)(F)C(F)(F)F)nc(OCC(F)(F)C(F)(F)C(F)(F)C(F)(F)F)n4)c3c2)cc1]

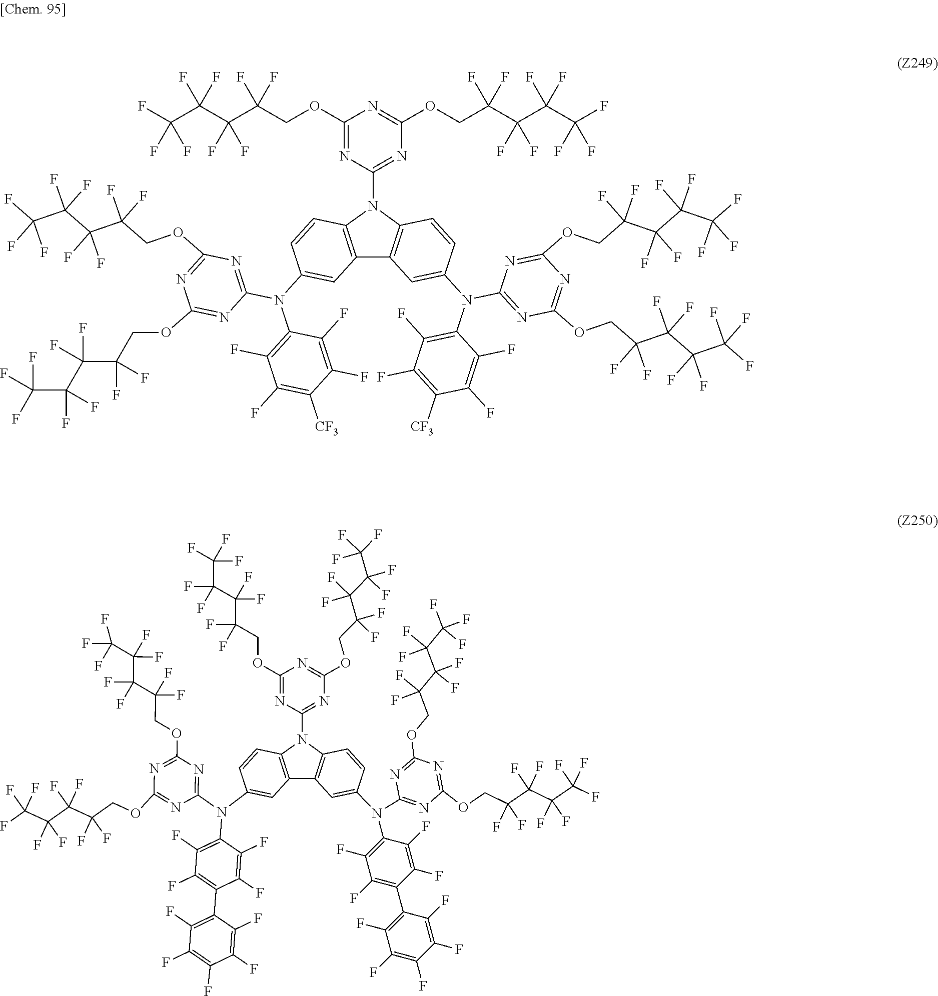

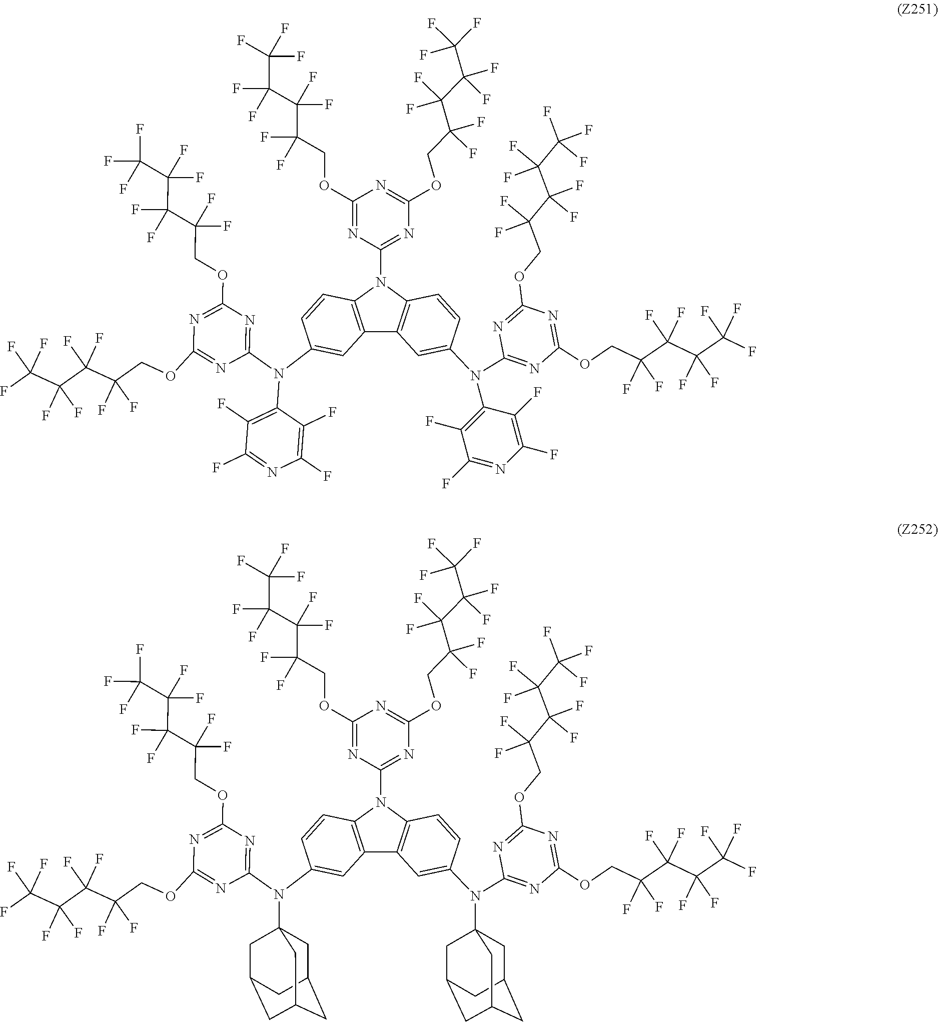

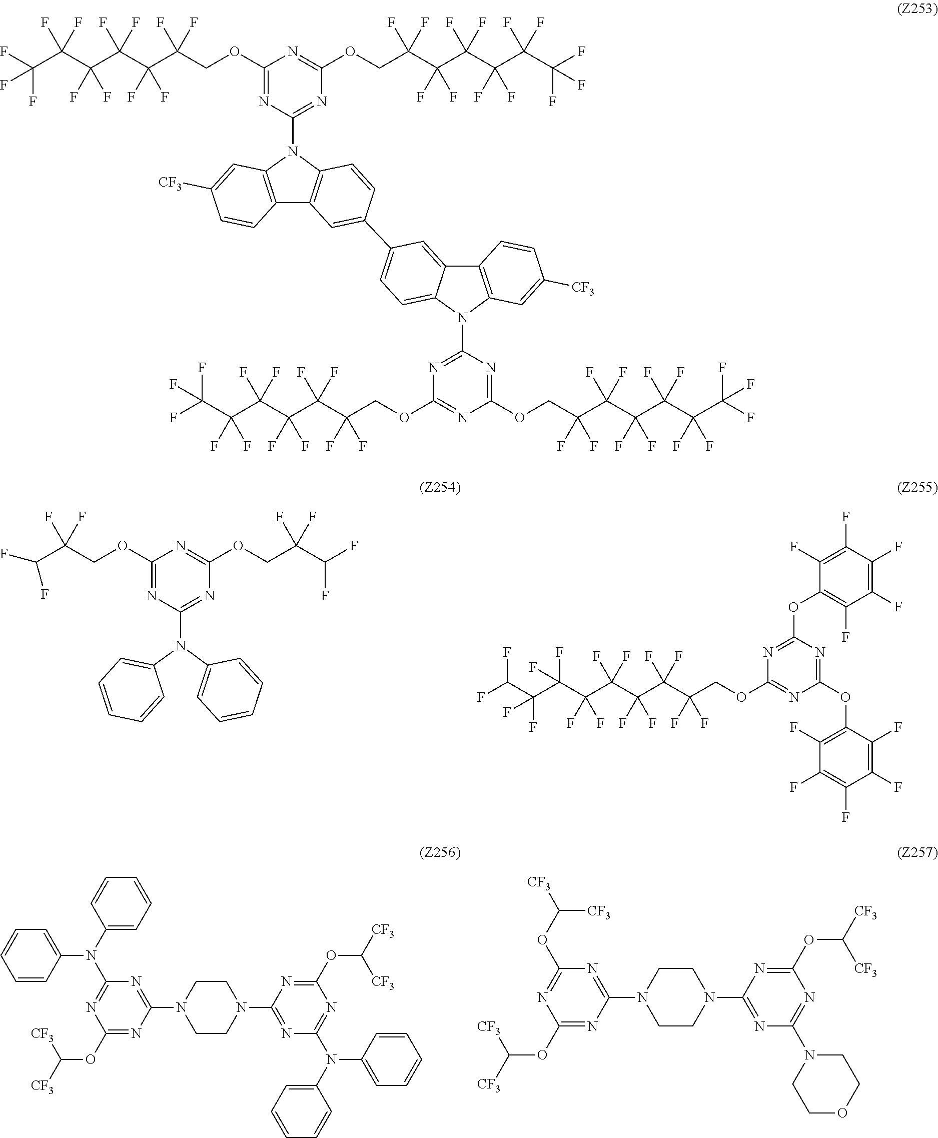

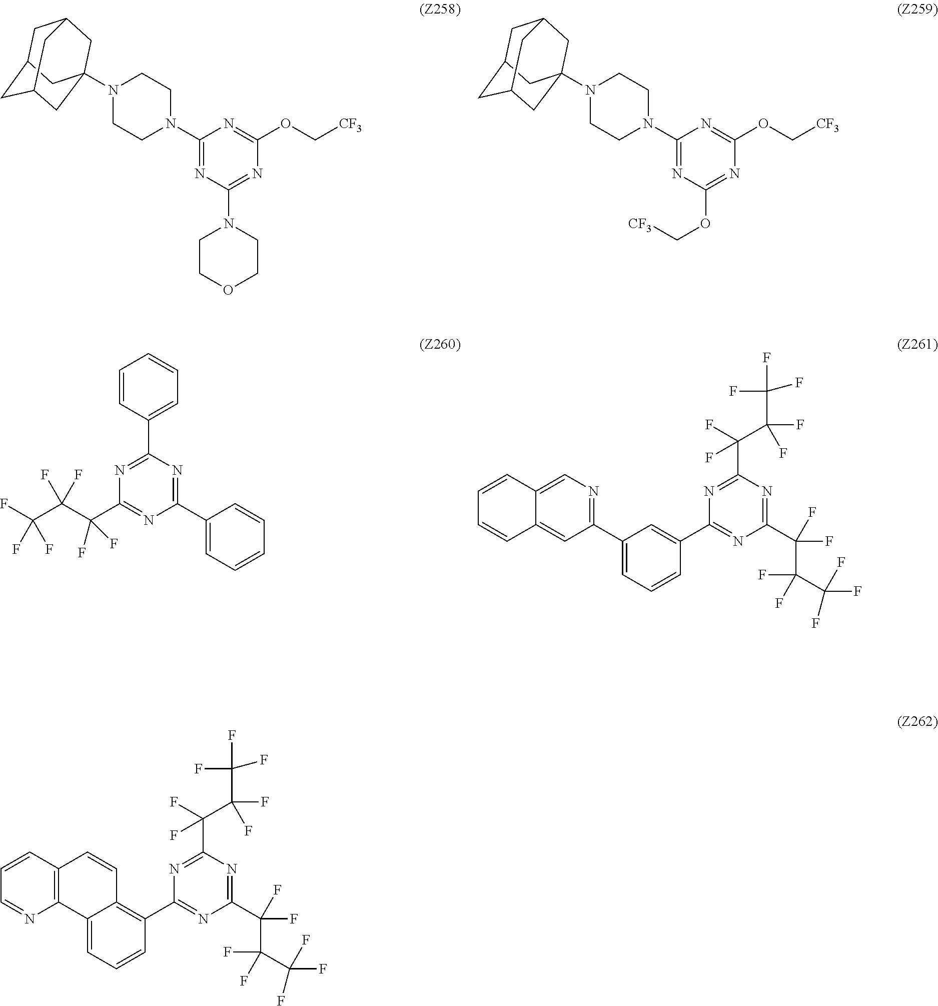 [SMILES: FC(F)(F)COc1nc(N2CCOCC2)nc(N2CCN(C34CC5CC(CC(C5)C3)C4)CC2)n1 ; FC(F)(F)COc1nc(OCC(F)(F)F)nc(N2CCN(C34CC5CC(CC(C5)C3)C4)CC2)n1 ; FC(F)(F)C(F)(F)C(F)(F)c1nc(-c2ccccc2)nc(-c2ccccc2)n1 ; FC(F)(F)C(F)(F)C(F)(F)c1nc(-c2cccc(-c3cc4ccccc4cn3)c2)nc(C(F)(F)C(F)(F)C(F)(F)F)n1 ; FC(F)(F)C(F)(F)C(F)(F)c1nc(-c2cccc3c2ccc2cccnc23)nc(C(F)(F)C(F)(F)C(F)(F)F)n1]

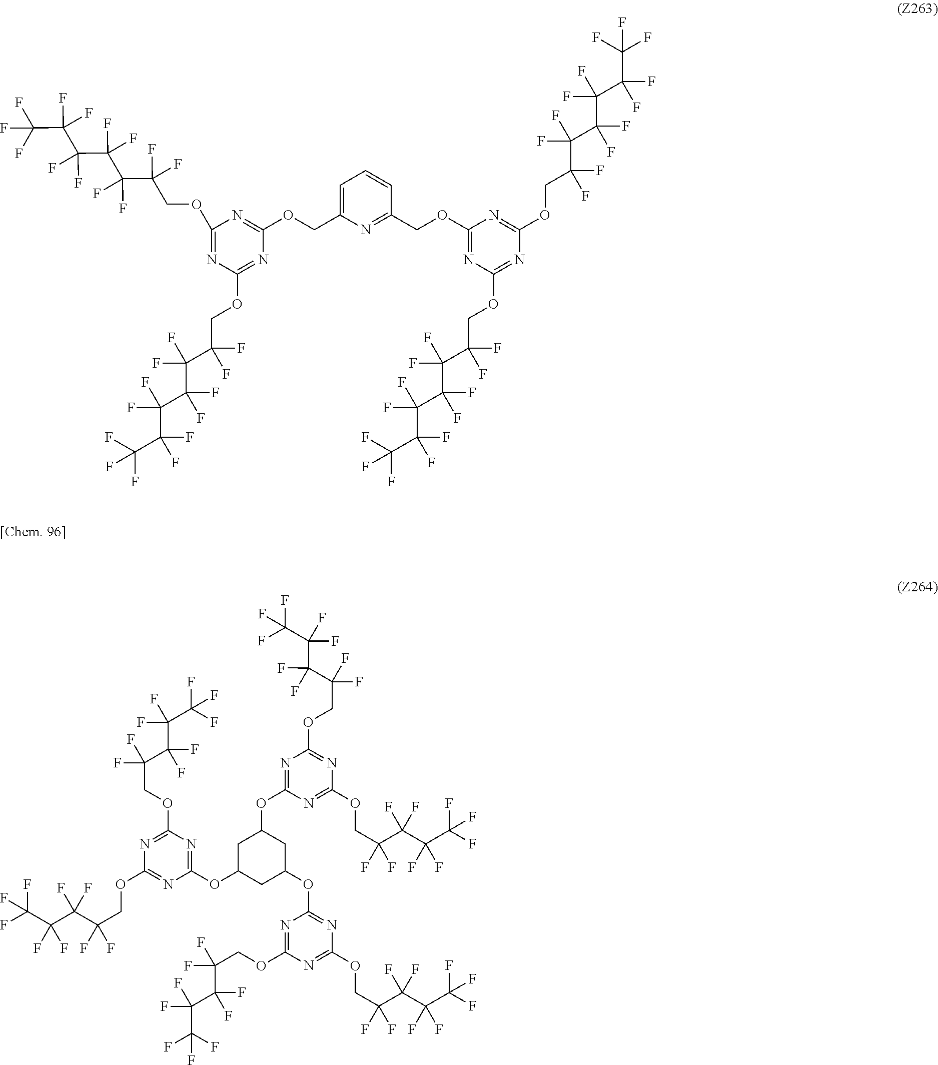 [SMILES: FC(F)(F)C(F)(F)C(F)(F)C(F)(F)C(F)(F)C(F)(F)COc1nc(OCc2cccc(COc3nc(OCC(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)F)n3)n2)nc(OCC(F)(F)C(F)(F)C(F)(F)C(F)(F)C(F)(F)C(F)(F)F)n1 ; FC(F)(F)C(F)(F)C(F)(F)C(F)(F)COc1nc(OCC(F)(F)C(F)(F)C(F)(F)C(F)(F)F)nc(OC2CC(Oc3nc(OCC(F)(F)C(F)(F)C(F)(F)C(F)(F)F)nc(OCC(F)(F)C(F)(F)C(F)(F)C(F)(F)F)n3)CC(Oc3nc(OCC(F)(F)C(F)(F)C(F)(F)C(F)(F)F)nc(OCC(F)(F)C(F)(F)C(F)(F)C(F)(F)F)n3)C2)n1]

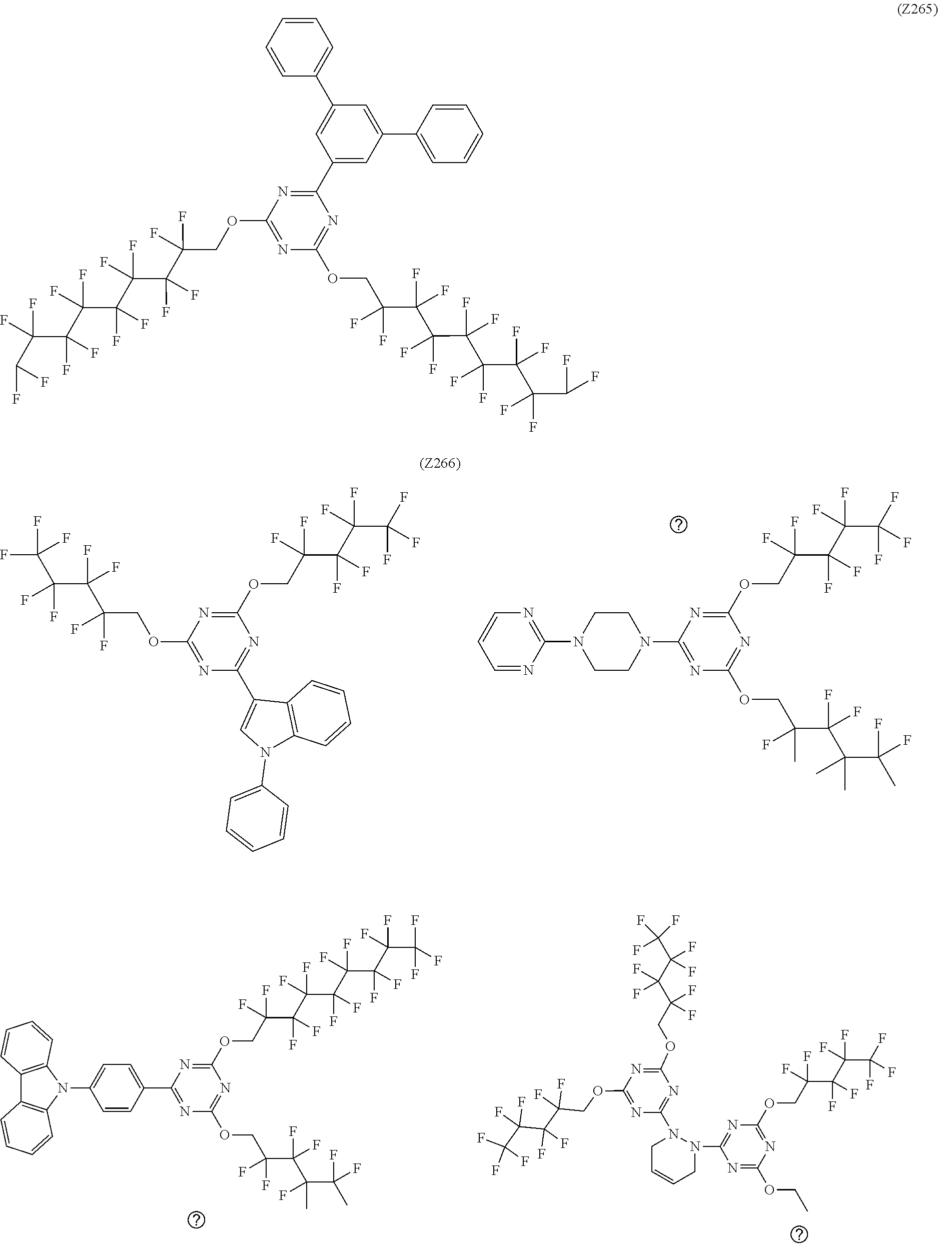

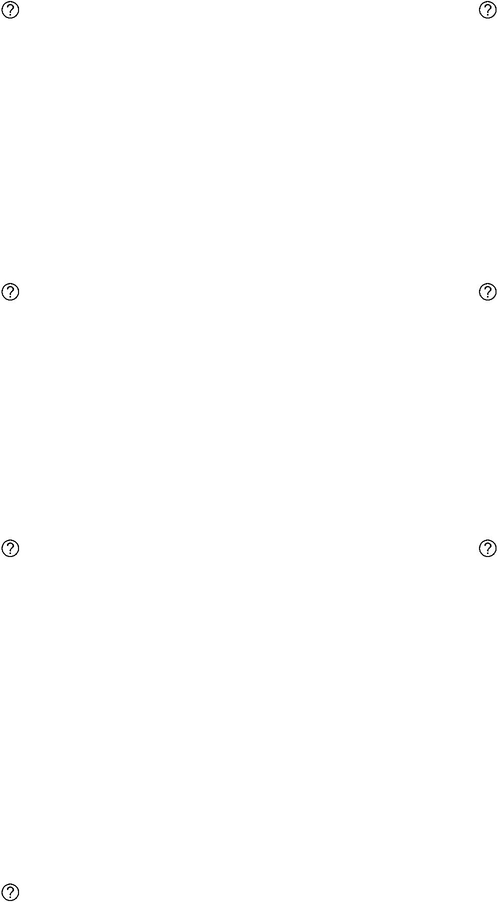

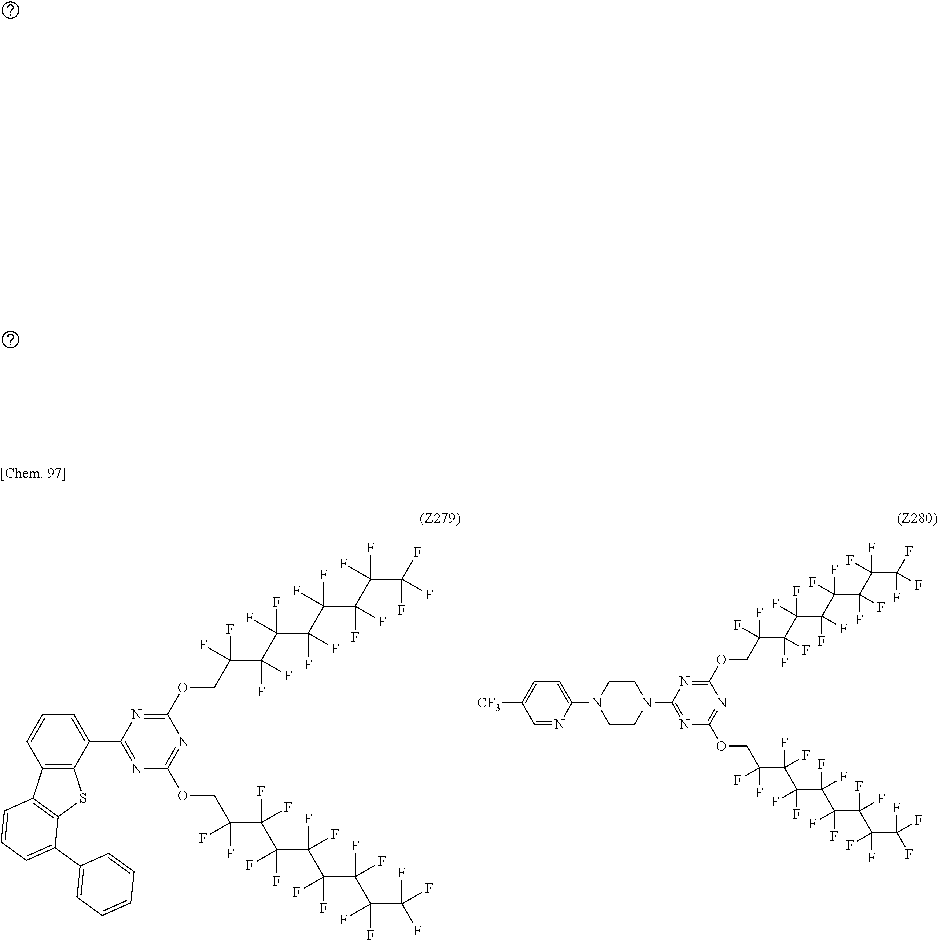 [SMILES: FC(F)(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)(F)F)nc(-c2cccc3c2sc2c(-c4ccccc4)cccc23)n1 ; FC(F)(F)c1ccc(N2CCN(c3nc(OCC(F)(F)C(F)(F)C(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)C(F)(F)C(F)(F)F)n3)CC2)nc1]

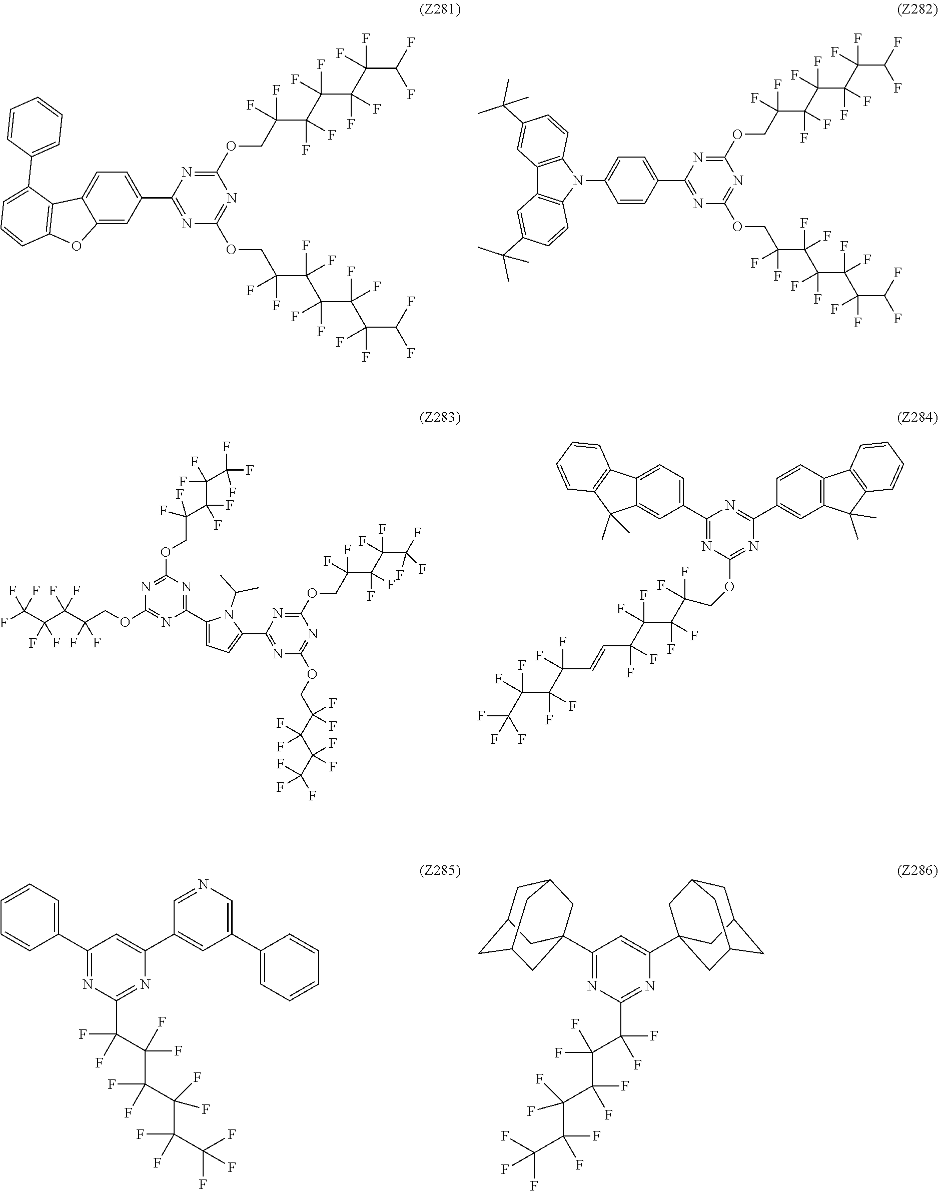

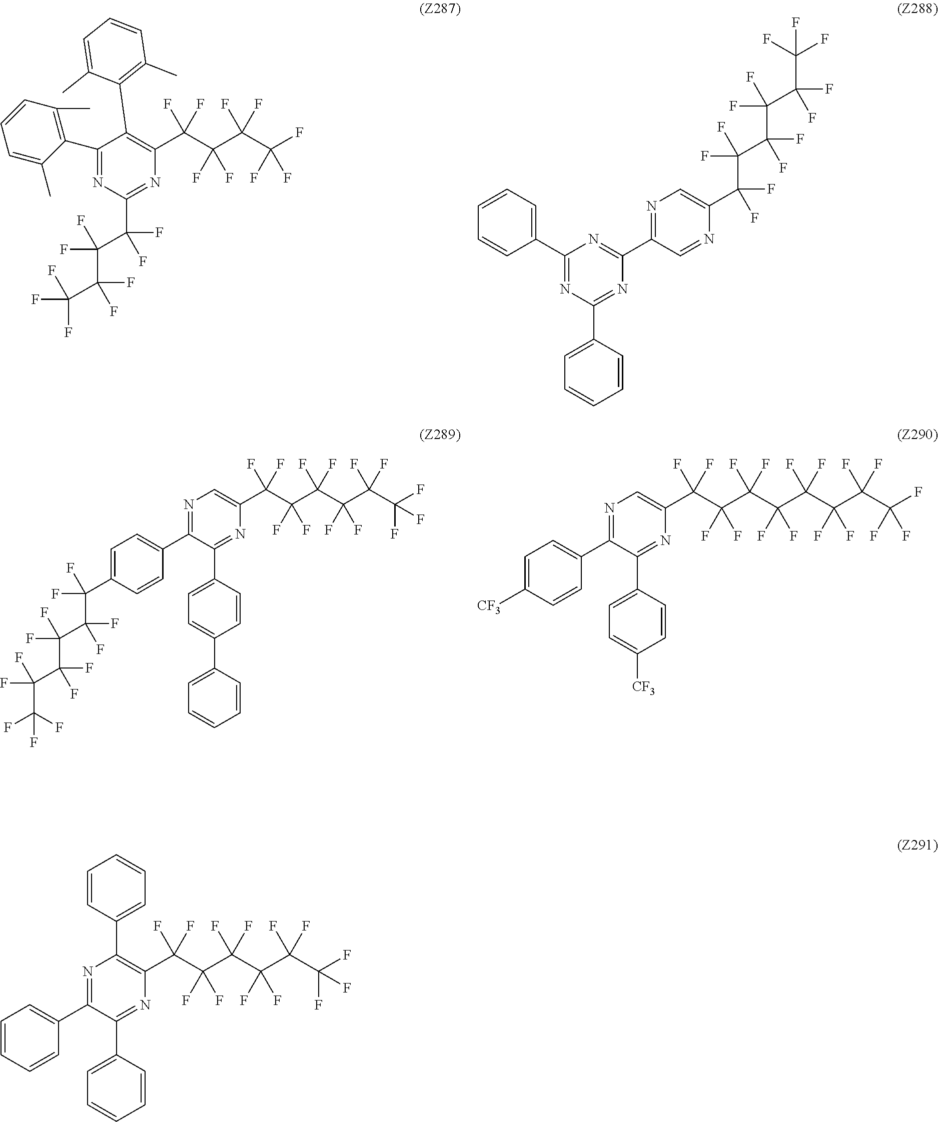

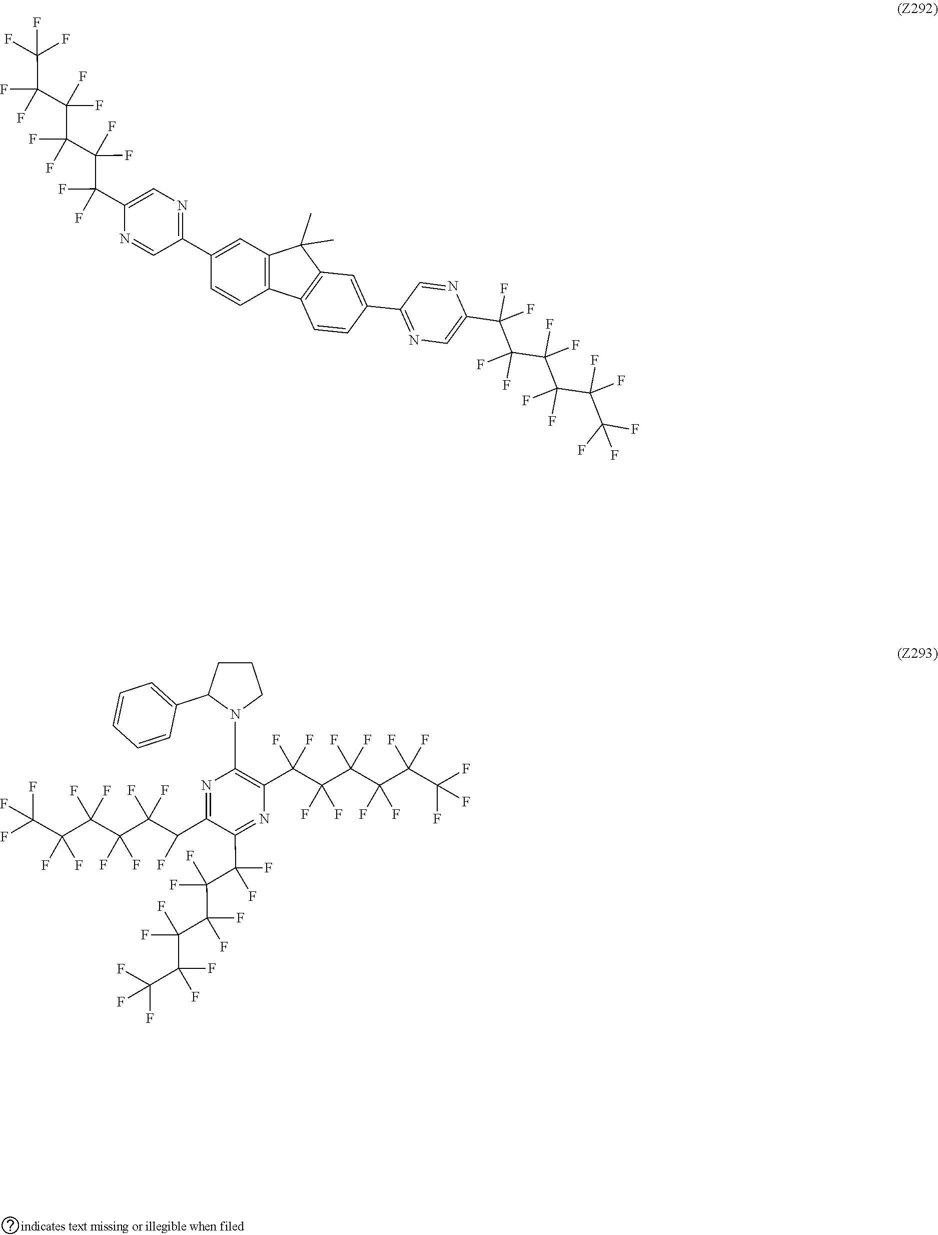 [SMILES: CC1(C)c2cc(-c3cnc(C(F)(F)C(F)(F)C(F)(F)C(F)(F)C(F)(F)C(F)(F)F)cn3)ccc2-c2ccc(-c3cnc(C(F)(F)C(F)(F)C(F)(F)C(F)(F)C(F)(F)C(F)(F)F)cn3)cc21 ; FC(c1nc(N2CCCC2c2ccccc2)c(C(F)(F)C(F)(F)C(F)(F)C(F)(F)C(F)(F)C(F)(F)F)nc1C(F)(F)C(F)(F)C(F)(F)C(F)(F)C(F)(F)C(F)(F)F)C(F)(F)C(F)(F)C(F)(F)C(F)(F)C(F)(F)F]

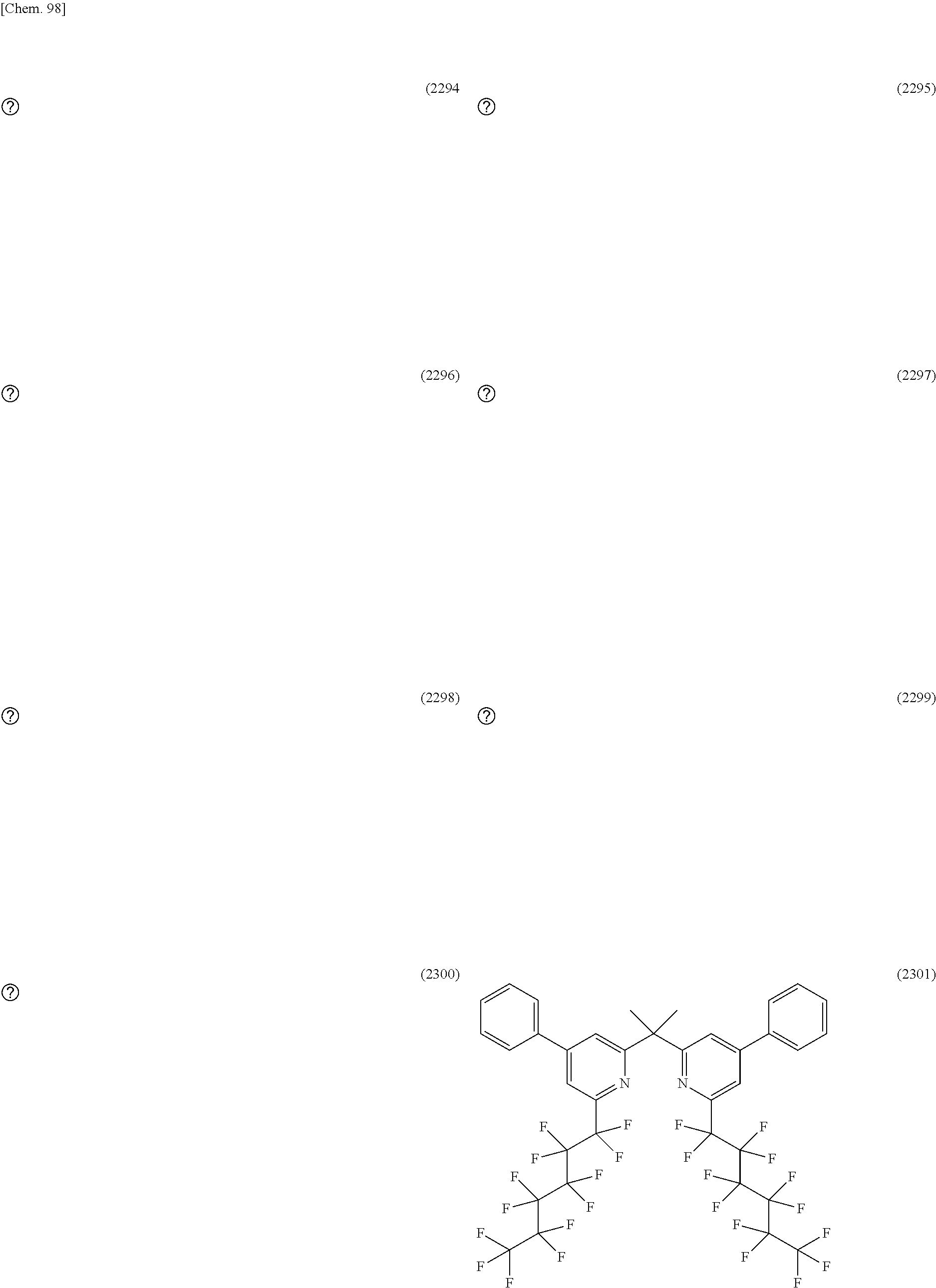 [SMILES: CC(C)(c1cc(-c2ccccc2)cc(C(F)(F)C(F)(F)C(F)(F)C(F)(F)C(F)(F)C(F)(F)F)n1)c1cc(-c2ccccc2)cc(C(F)(F)C(F)(F)C(F)(F)C(F)(F)C(F)(F)C(F)(F)F)n1]

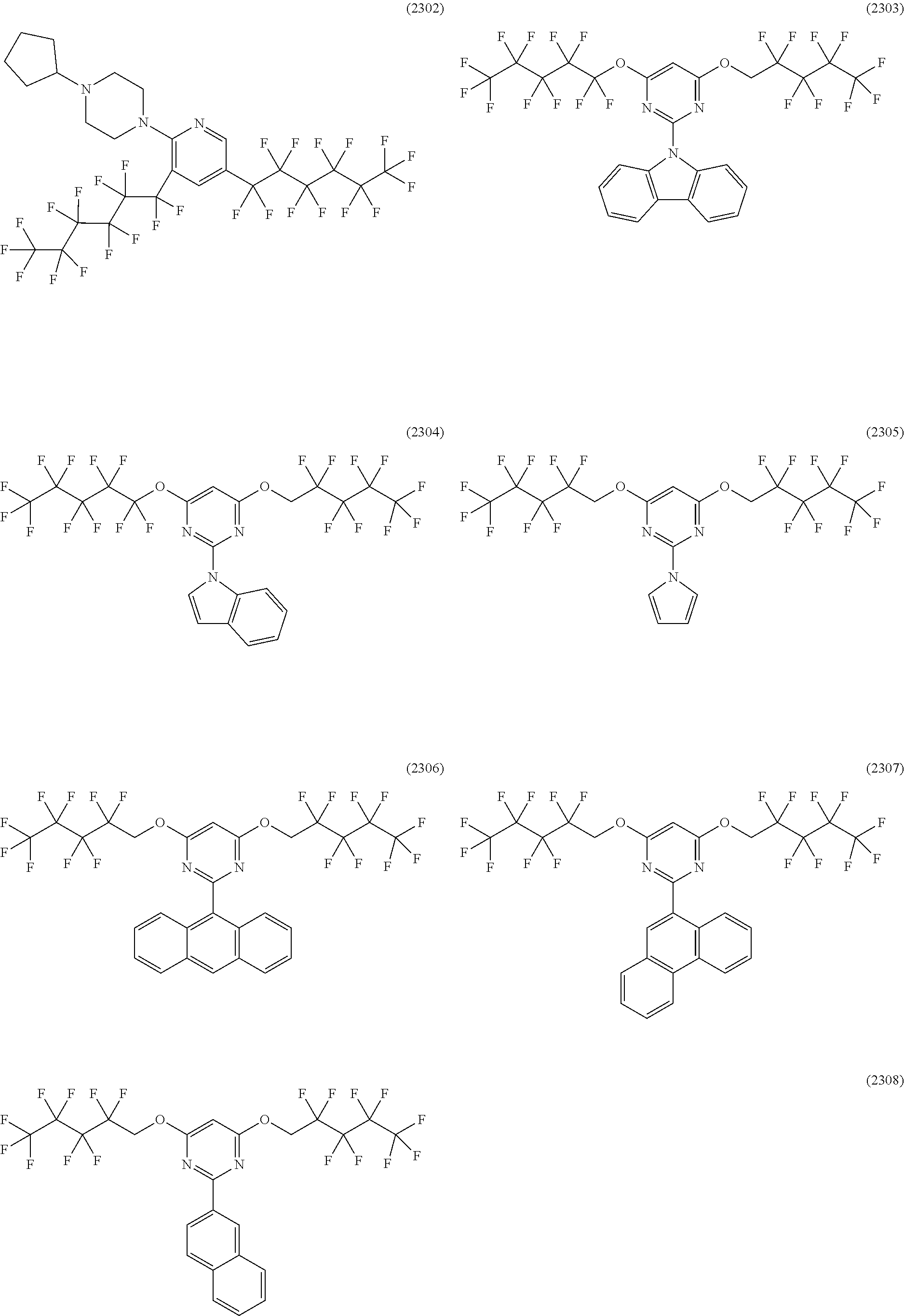

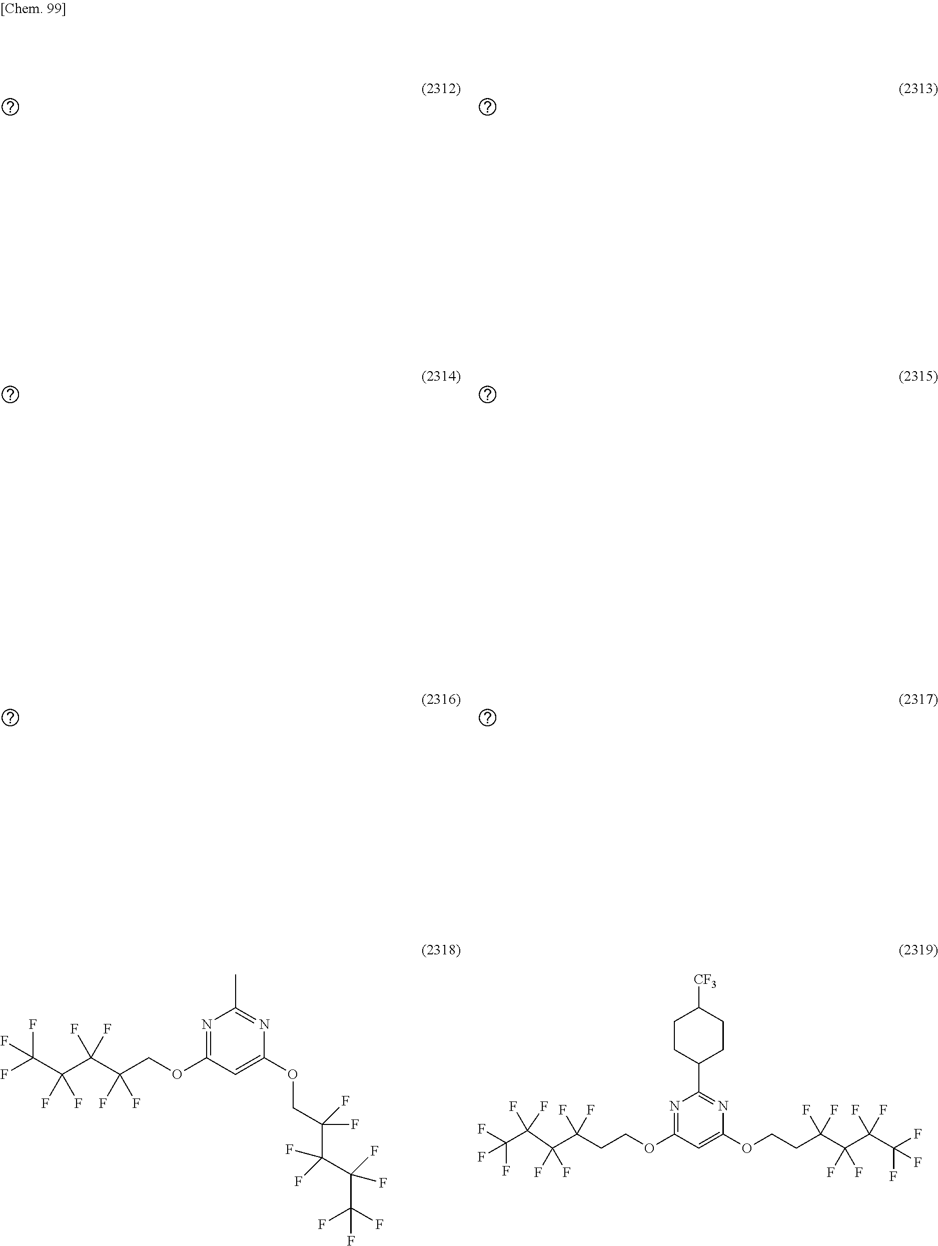 [SMILES: Cc1nc(OCC(F)(F)C(F)(F)C(F)(F)C(F)(F)F)cc(OCC(F)(F)C(F)(F)C(F)(F)C(F)(F)F)n1 ; FC(F)(F)C1CCC(c2nc(OCCC(F)(F)C(F)(F)C(F)(F)C(F)(F)F)cc(OCCC(F)(F)C(F)(F)C(F)(F)C(F)(F)F)n2)CC1]

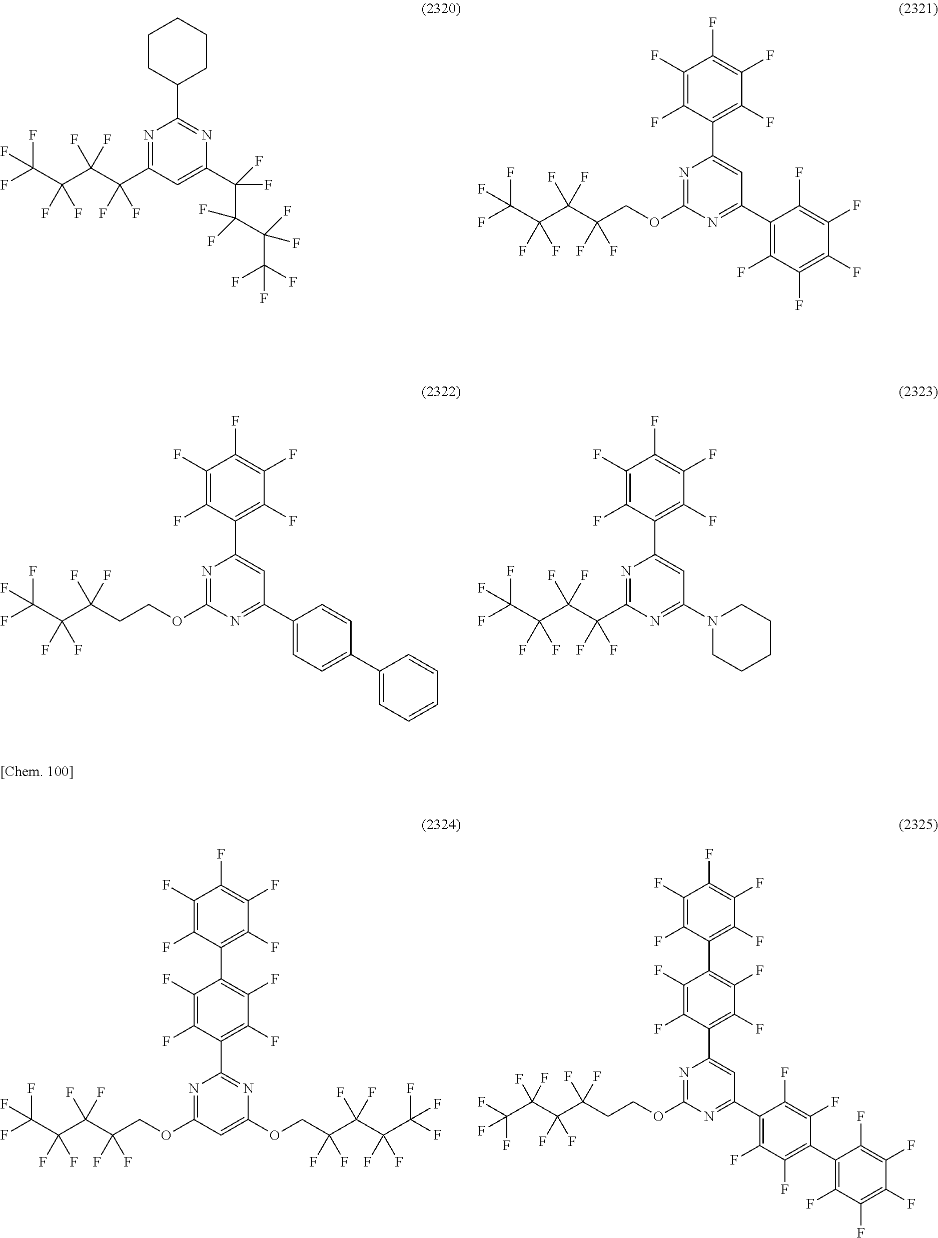

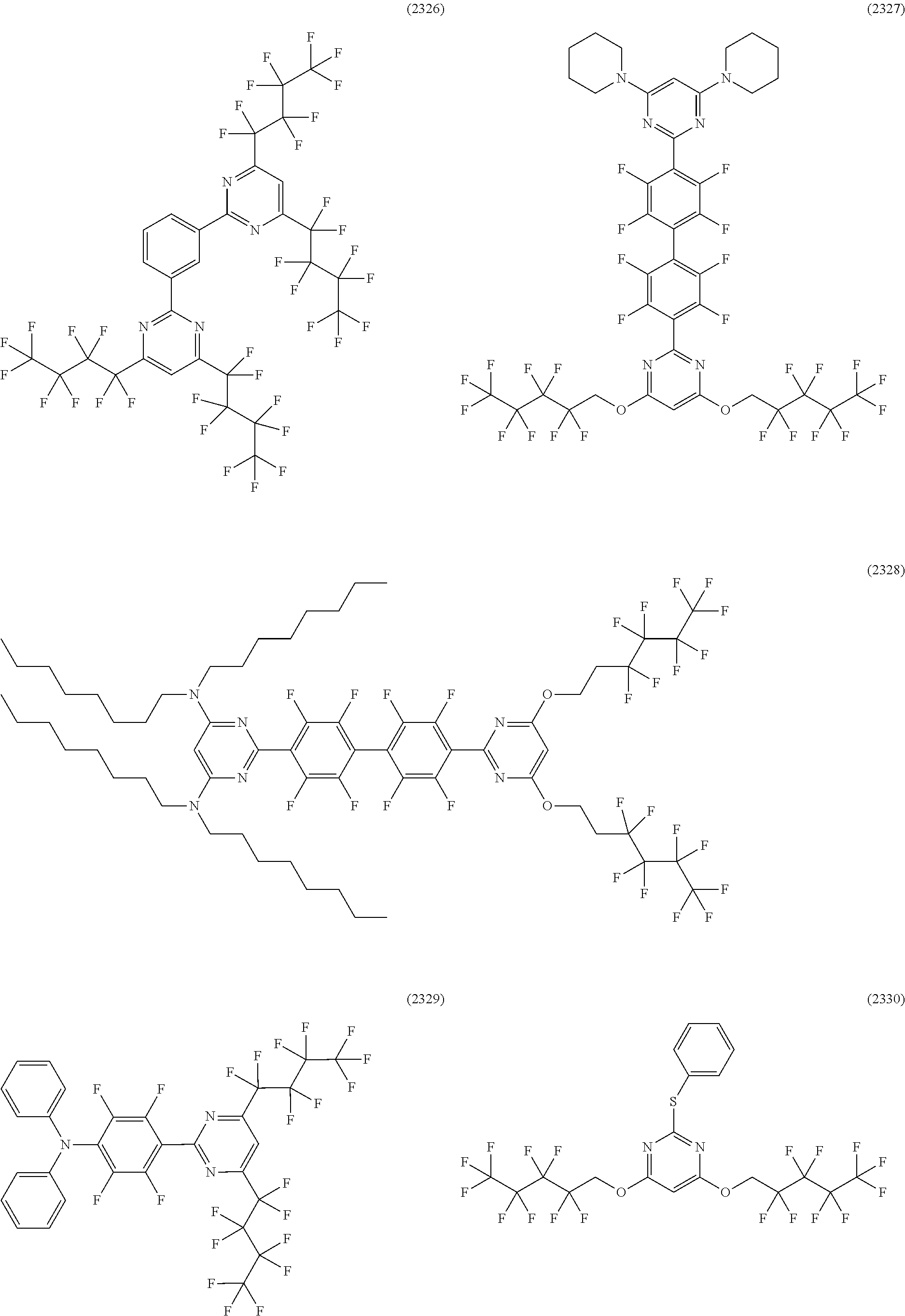

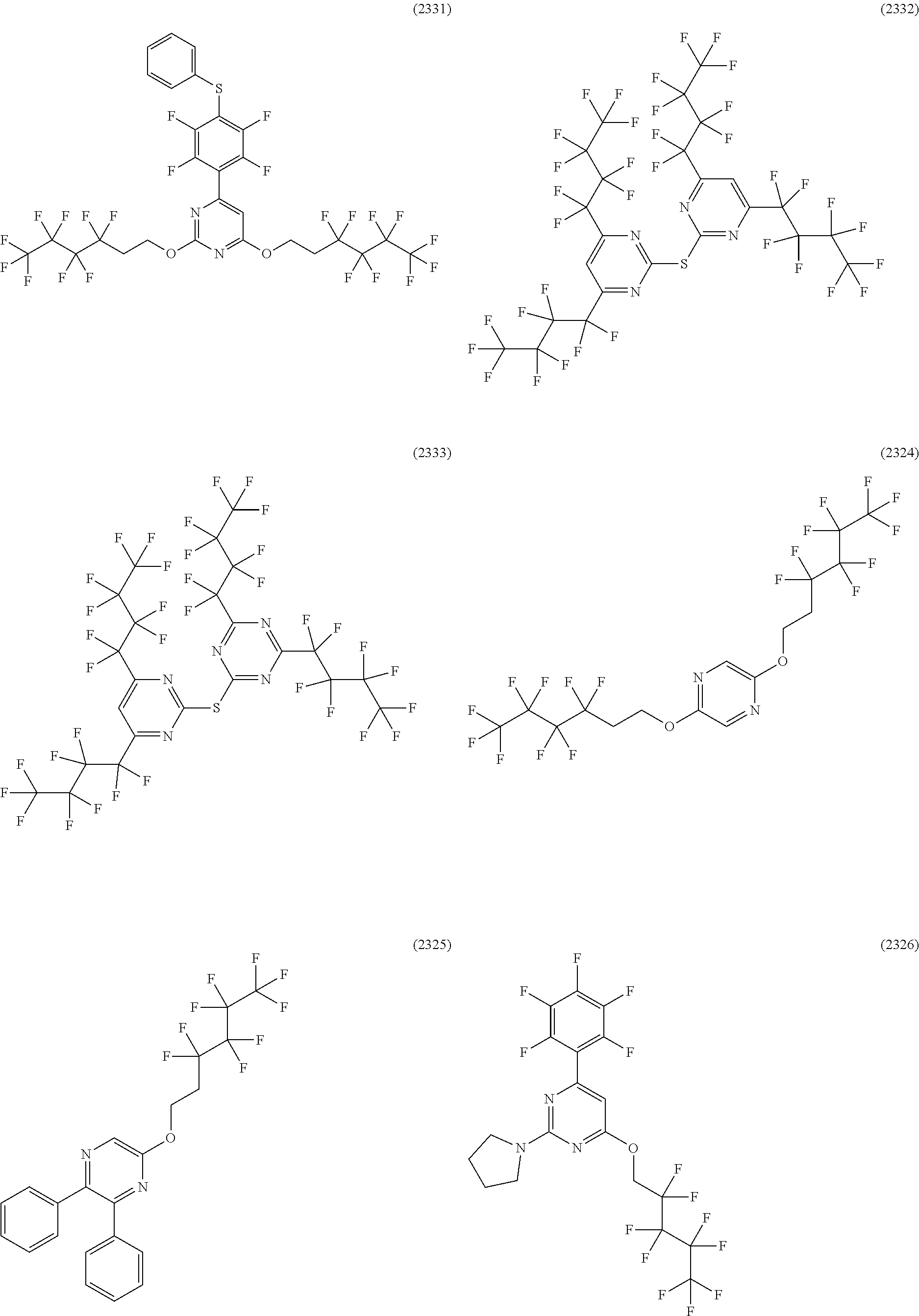

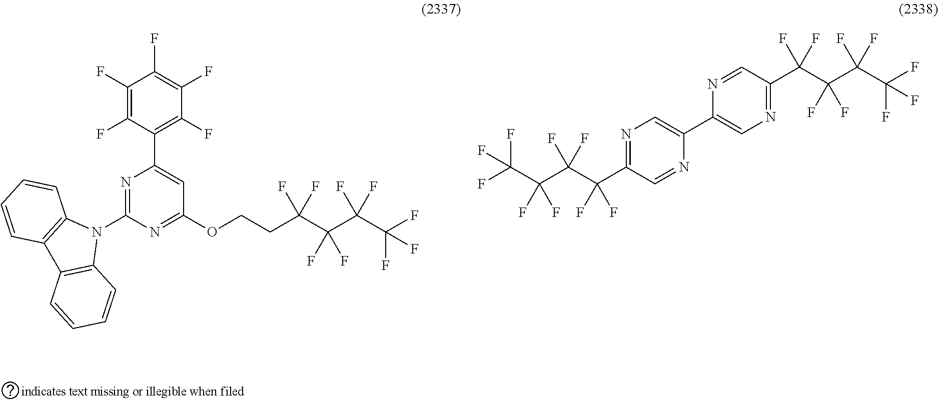 [SMILES: Fc1c(F)c(F)c(-c2cc(OCCC(F)(F)C(F)(F)C(F)(F)C(F)(F)F)nc(-n3c4ccccc4c4ccccc43)n2)c(F)c1F ; FC(F)(F)C(F)(F)C(F)(F)C(F)(F)c1cnc(-c2cnc(C(F)(F)C(F)(F)C(F)(F)C(F)(F)F)cn2)cn1]

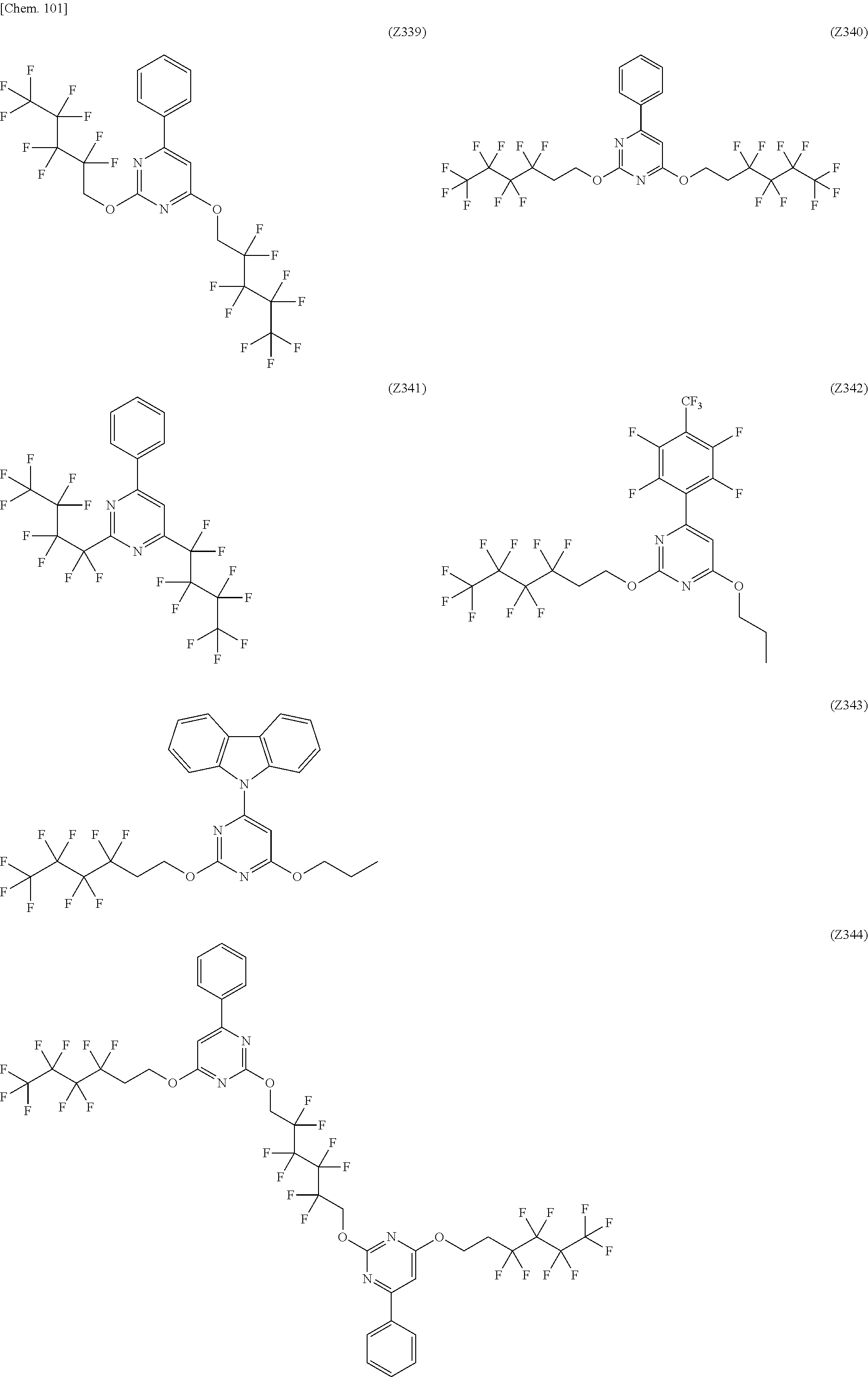

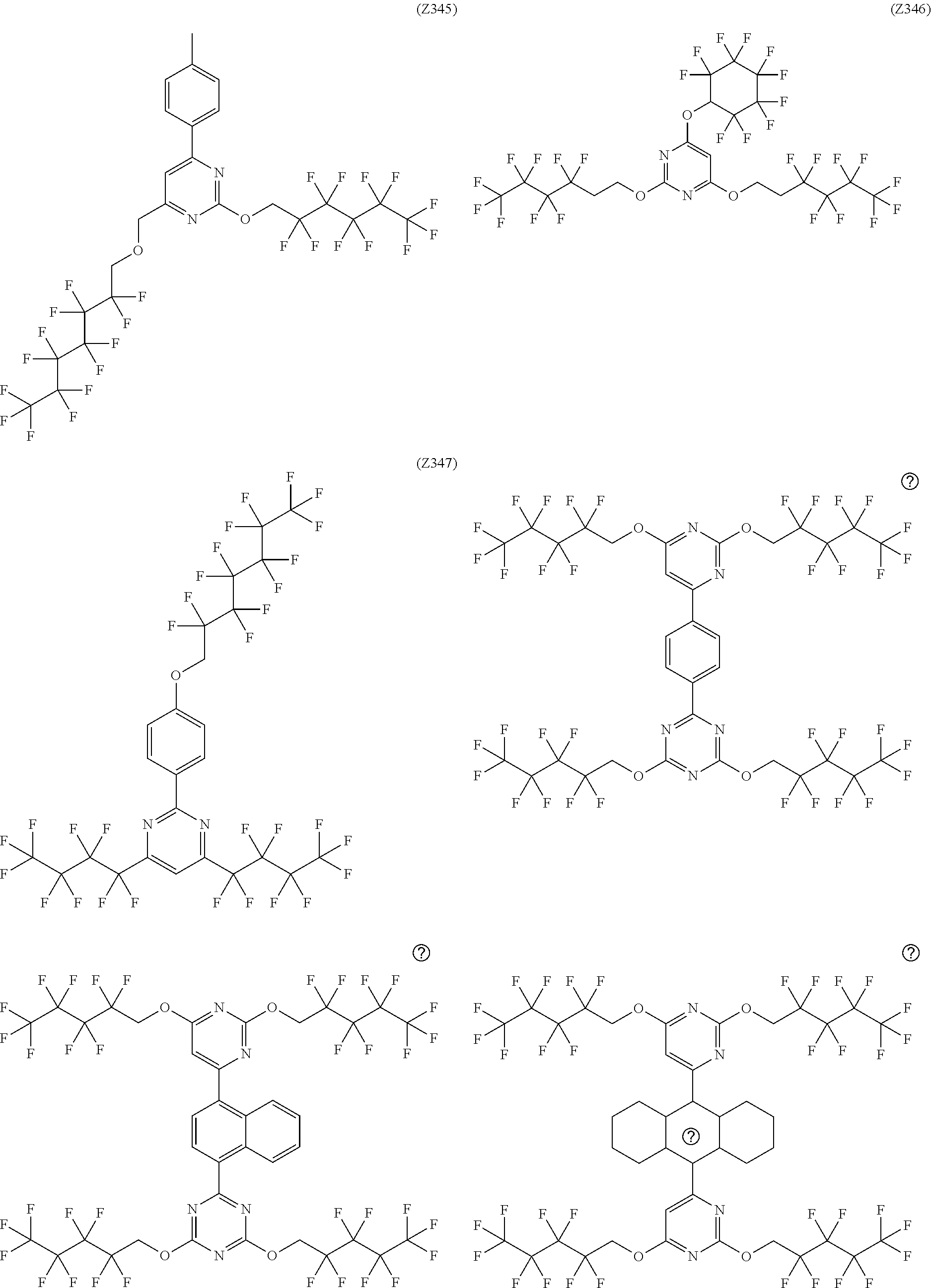

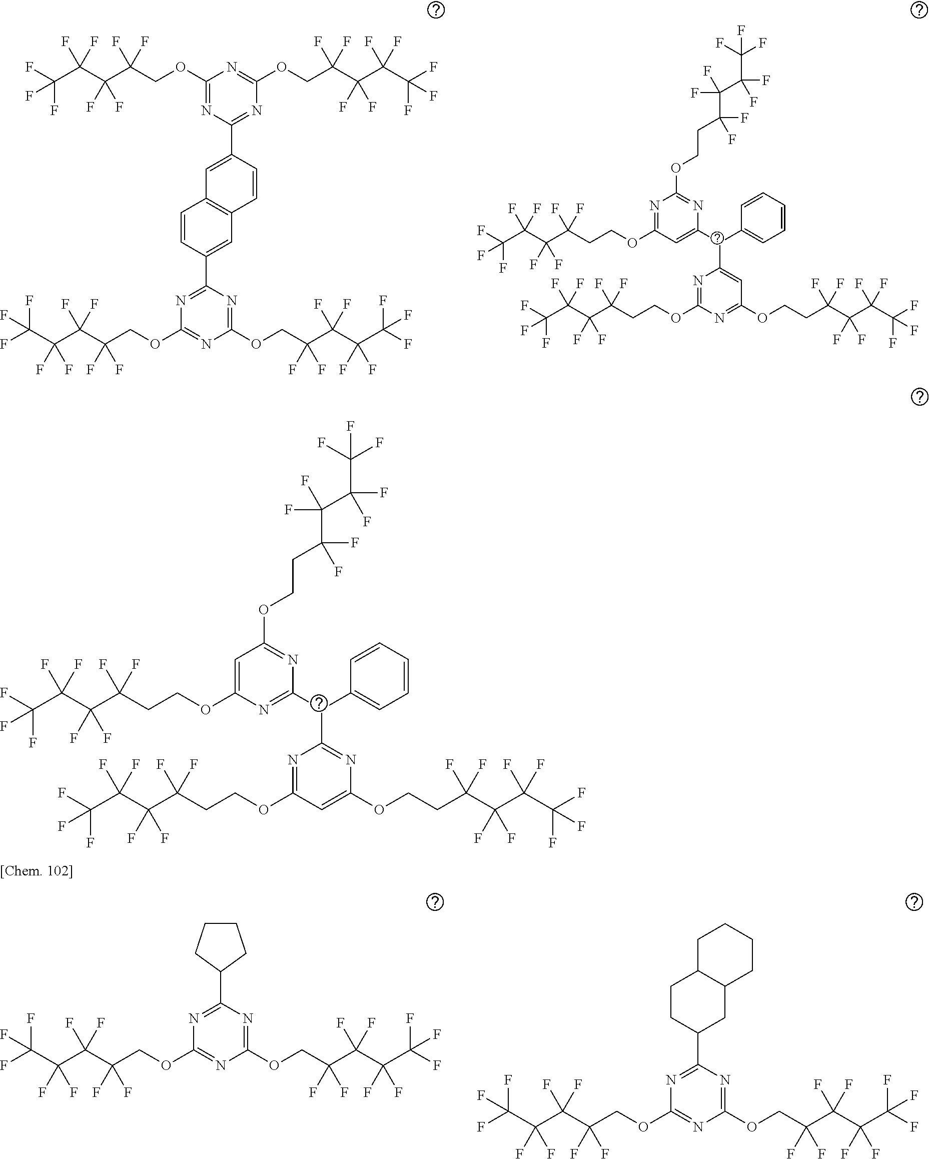

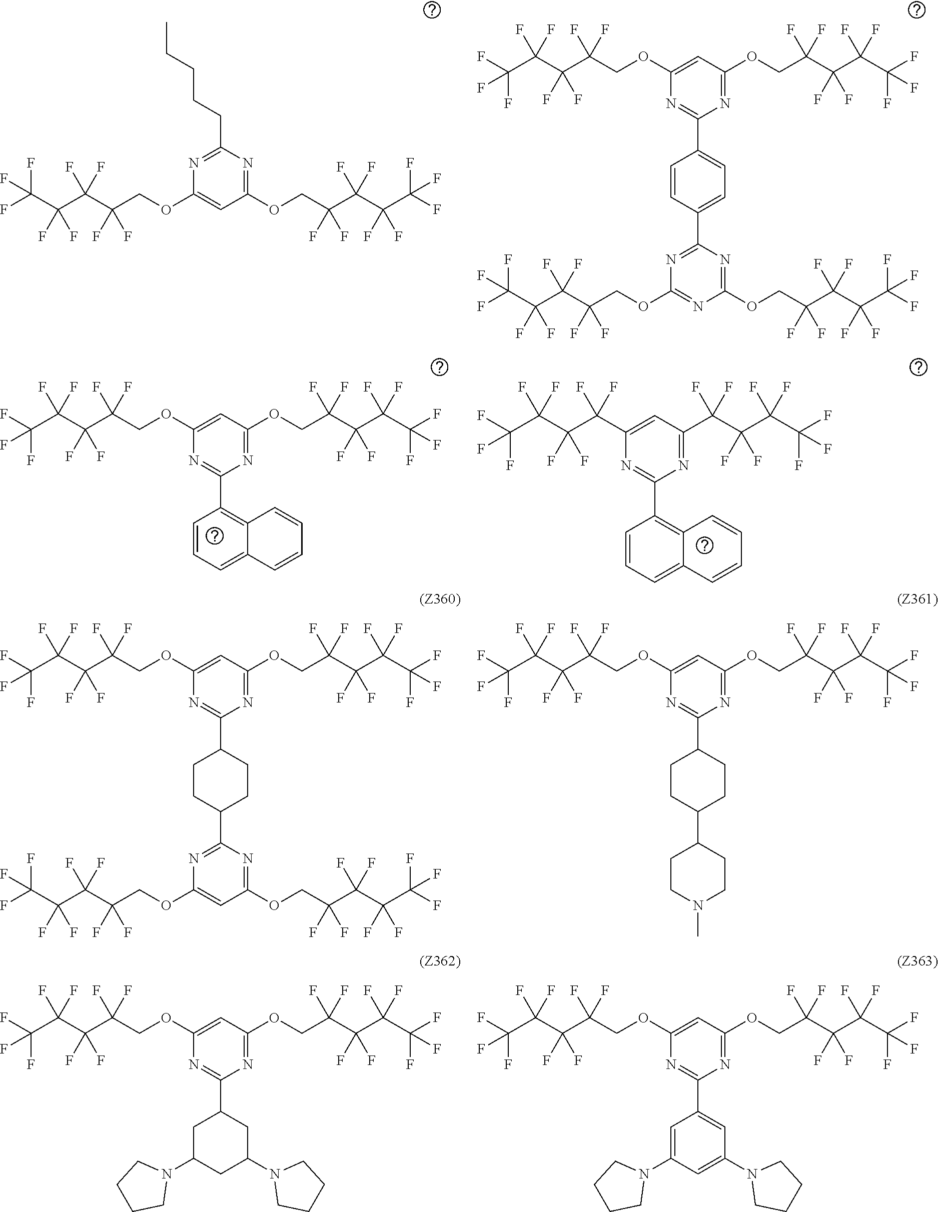

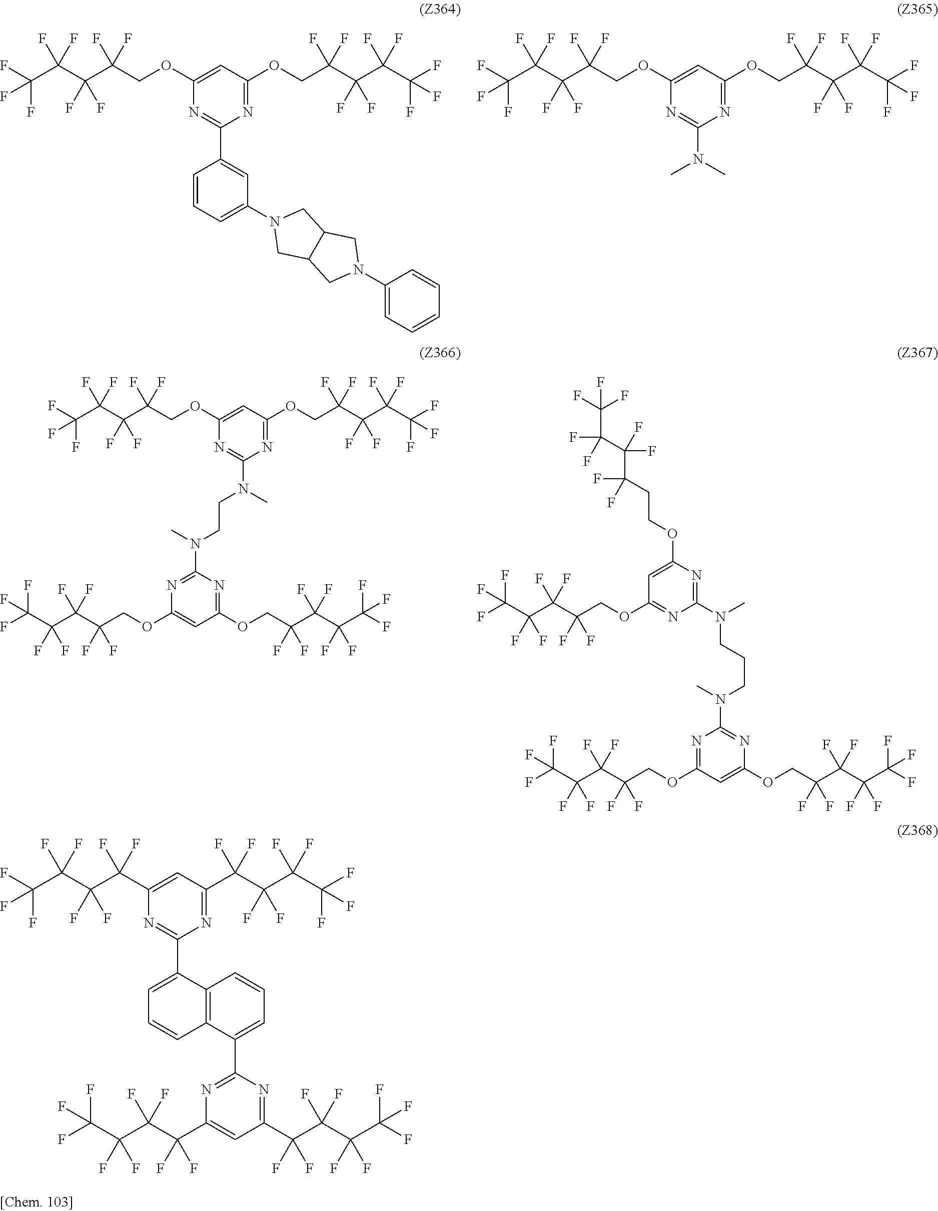

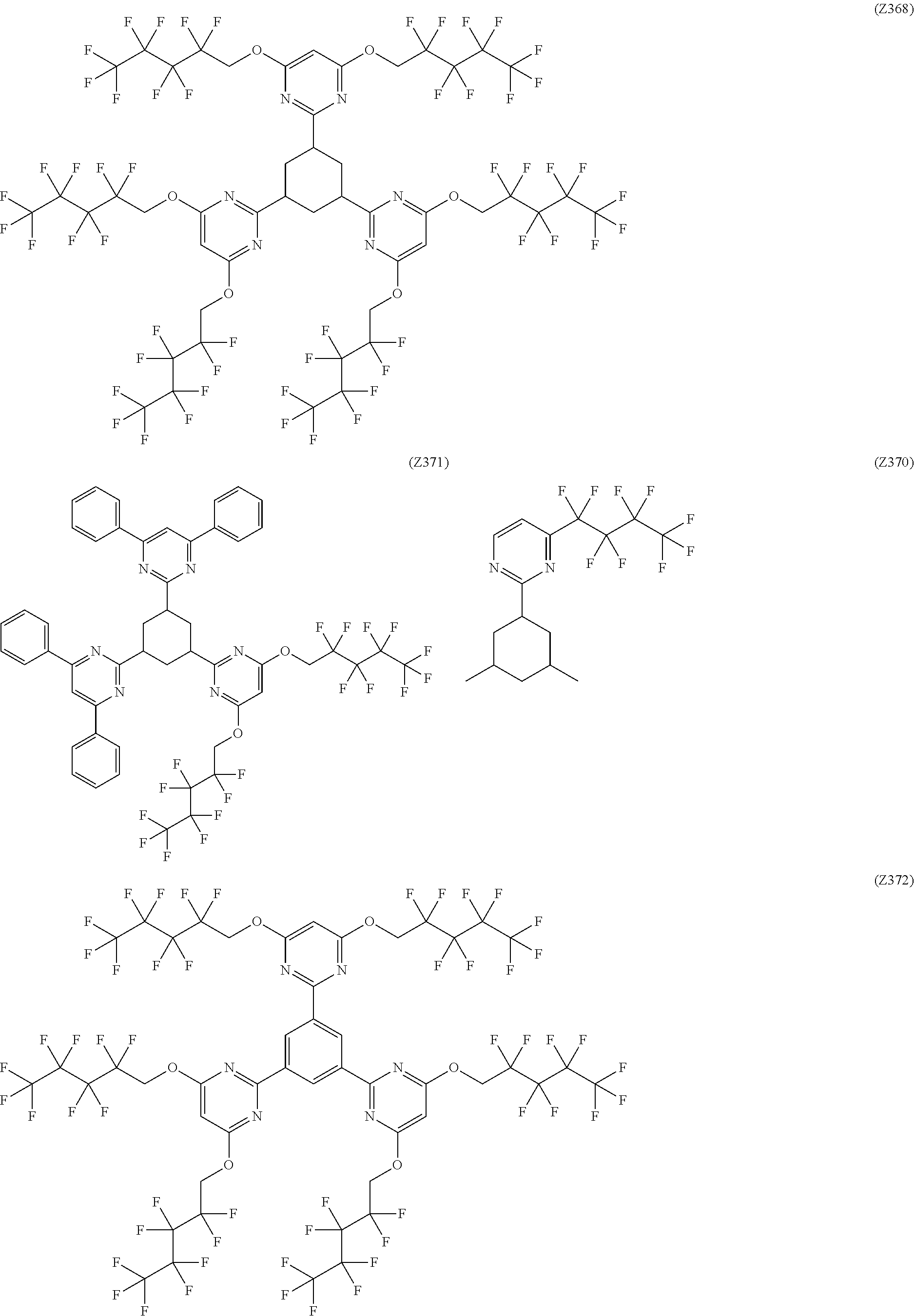

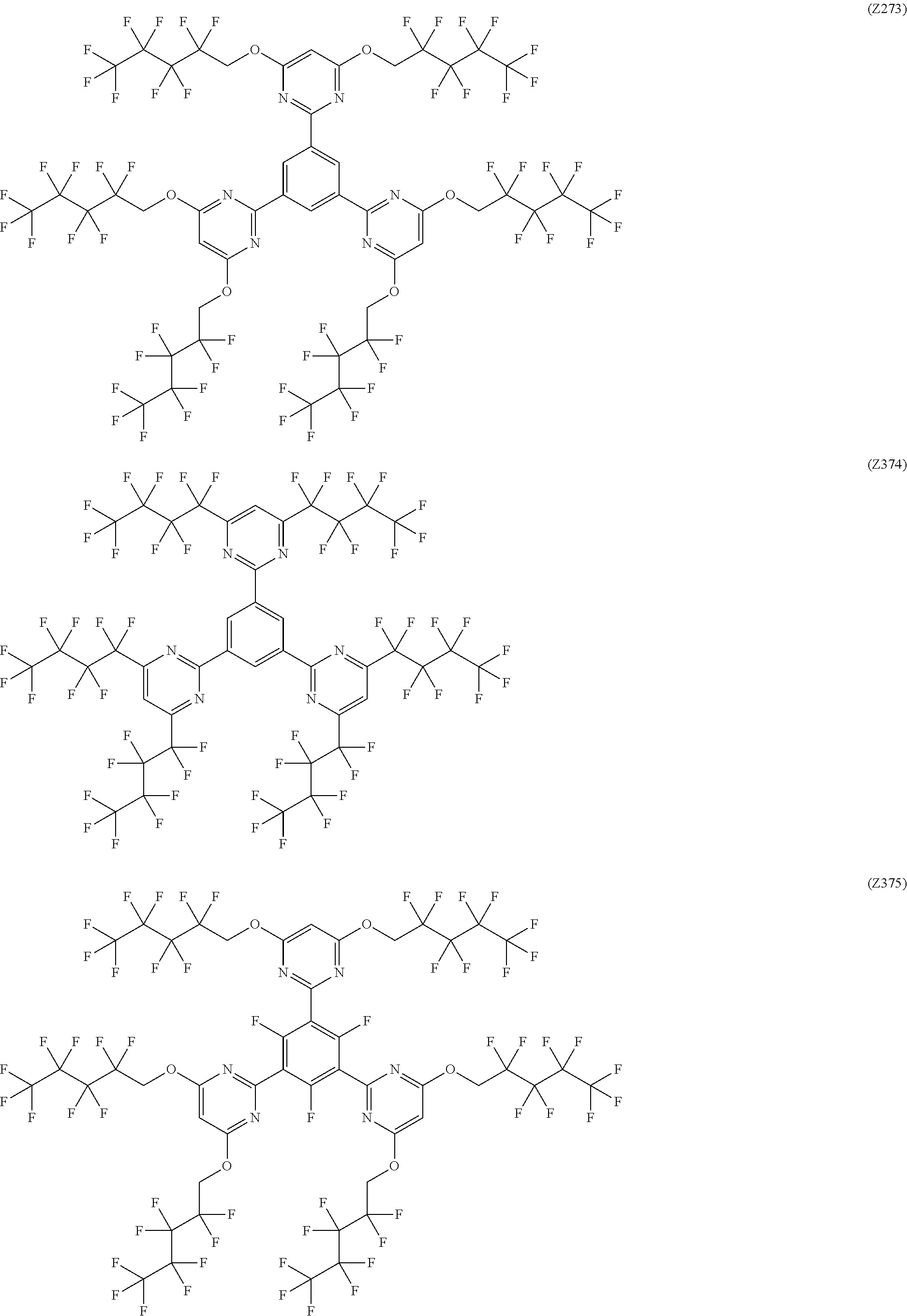

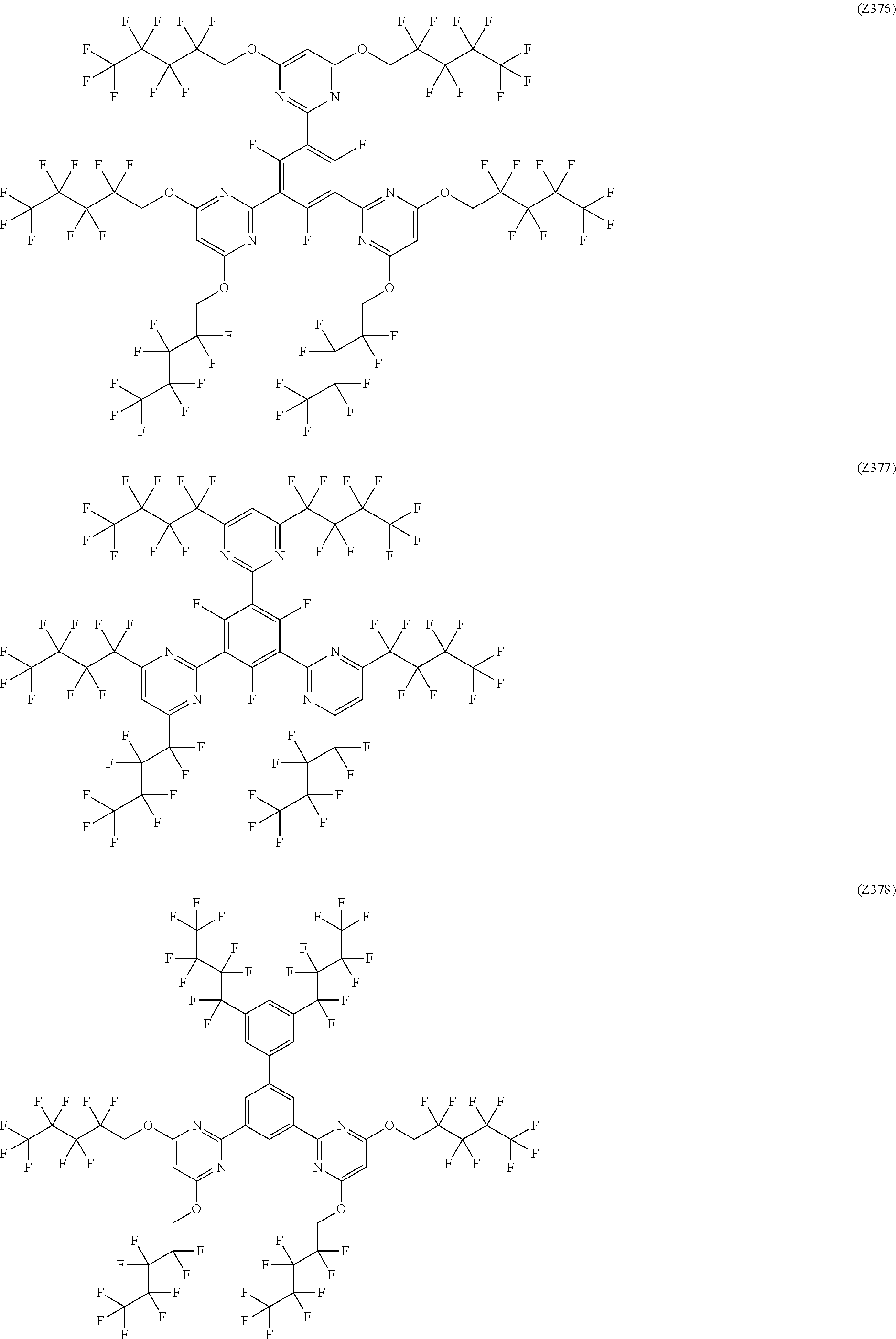

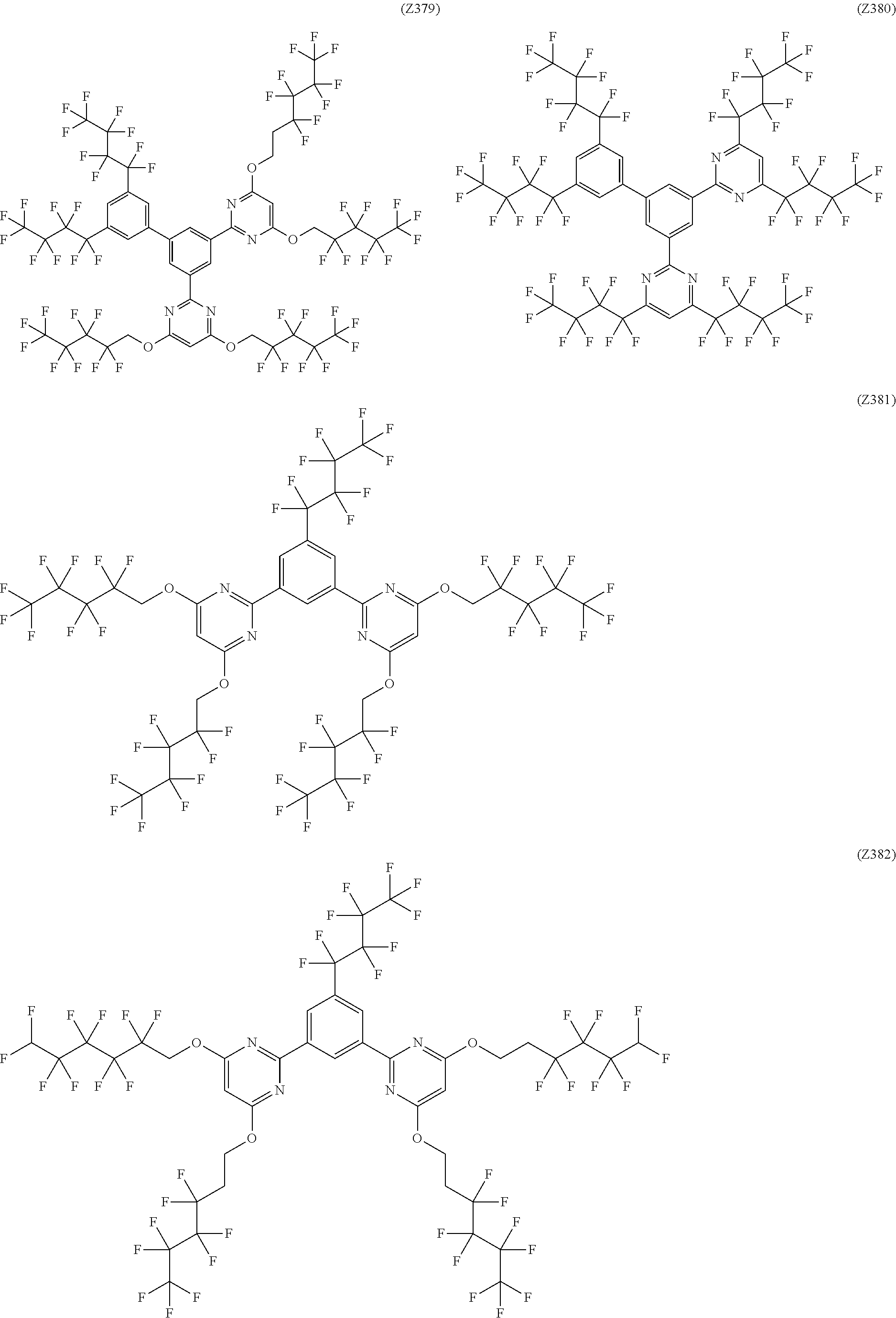

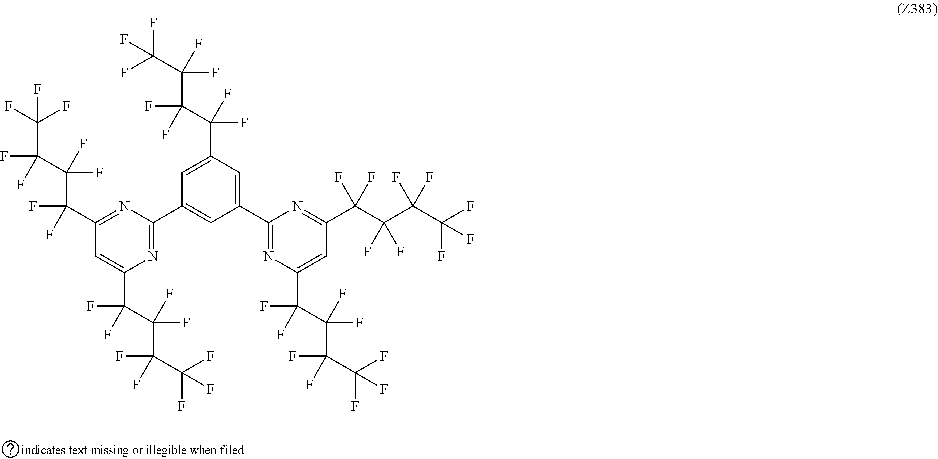 [SMILES: FC(F)(F)C(F)(F)C(F)(F)C(F)(F)c1cc(-c2nc(C(F)(F)C(F)(F)C(F)(F)C(F)(F)F)cc(C(F)(F)C(F)(F)C(F)(F)C(F)(F)F)n2)cc(-c2nc(C(F)(F)C(F)(F)C(F)(F)C(F)(F)F)cc(C(F)(F)C(F)(F)C(F)(F)C(F)(F)F)n2)c1]

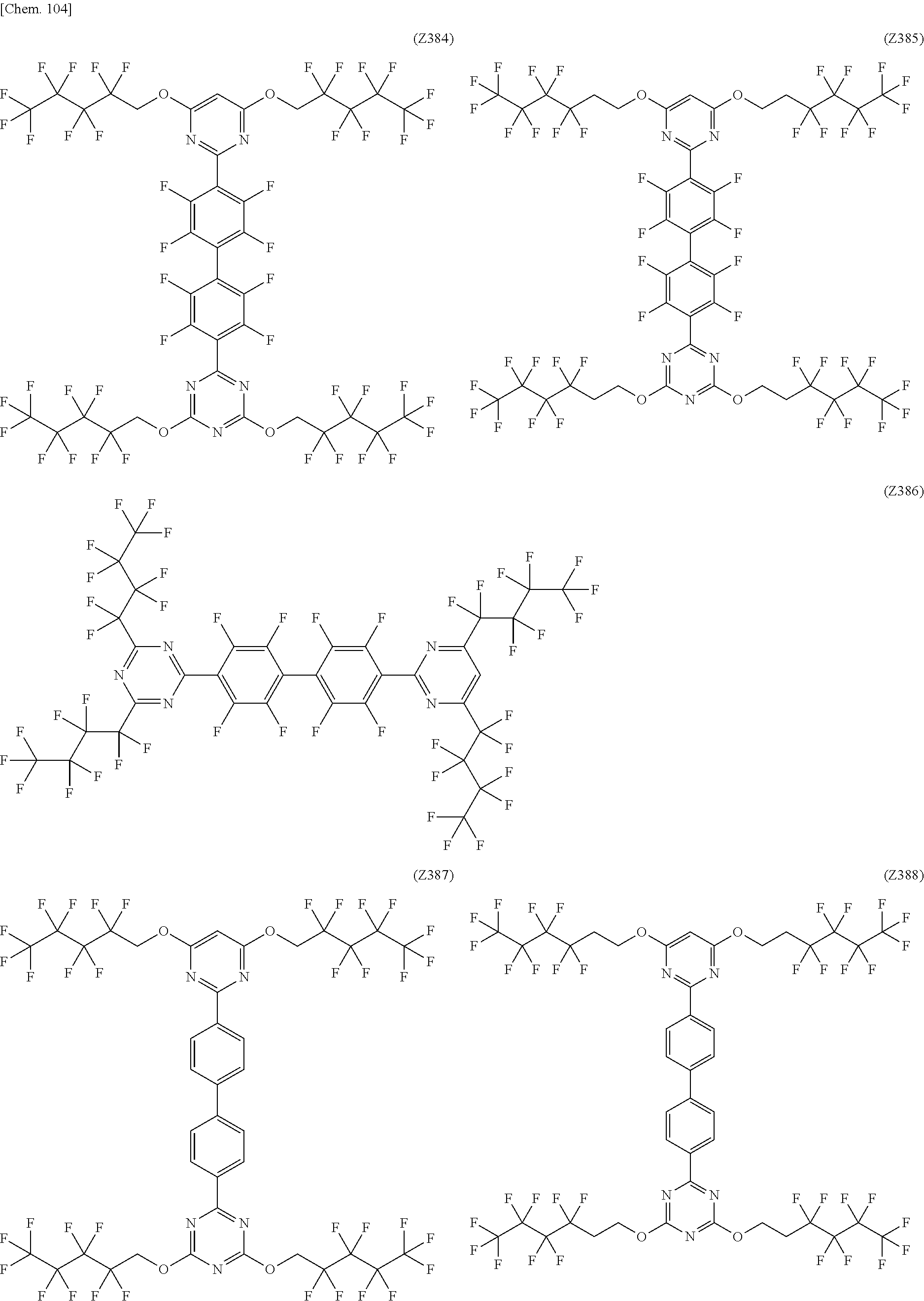

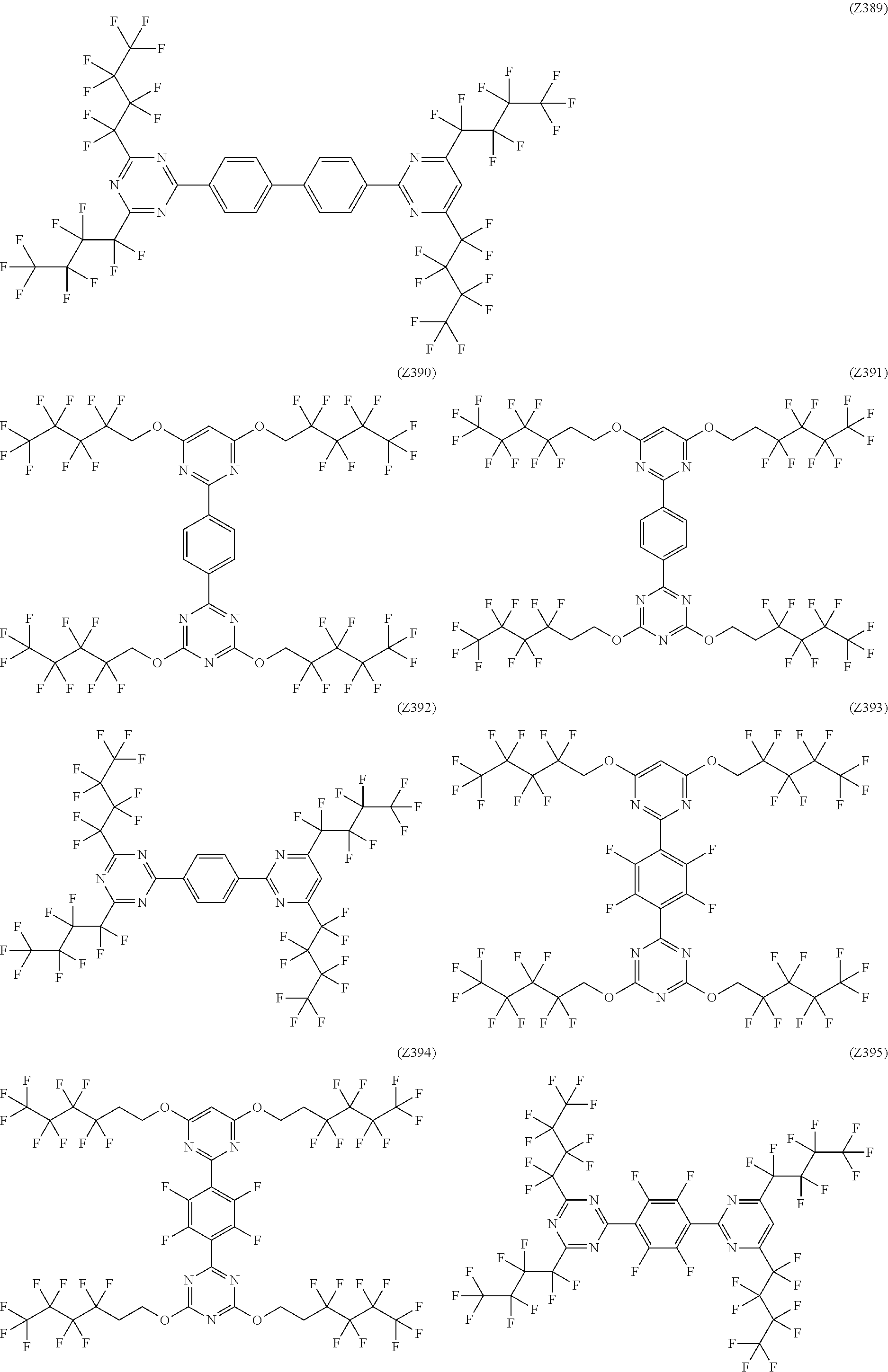

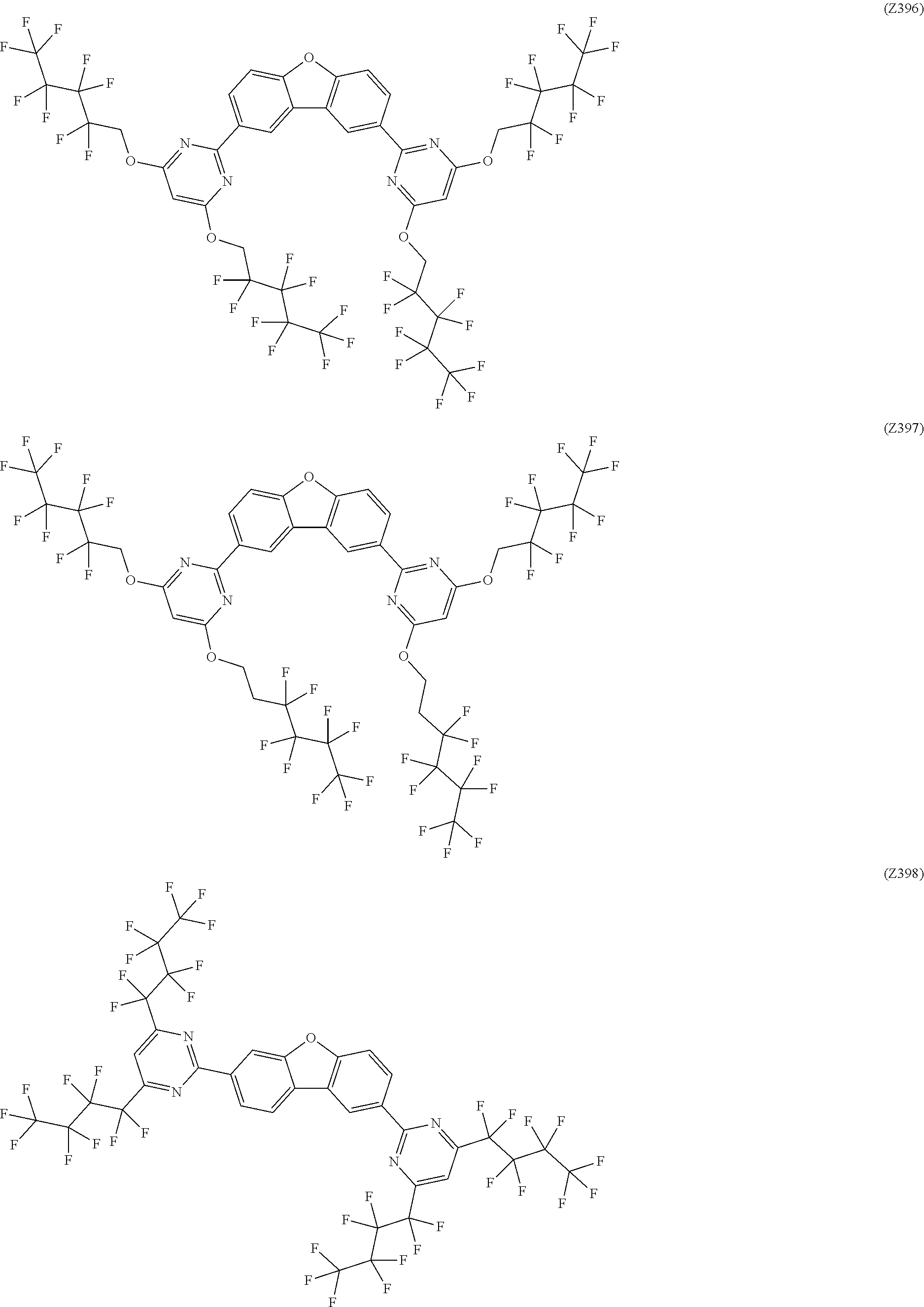

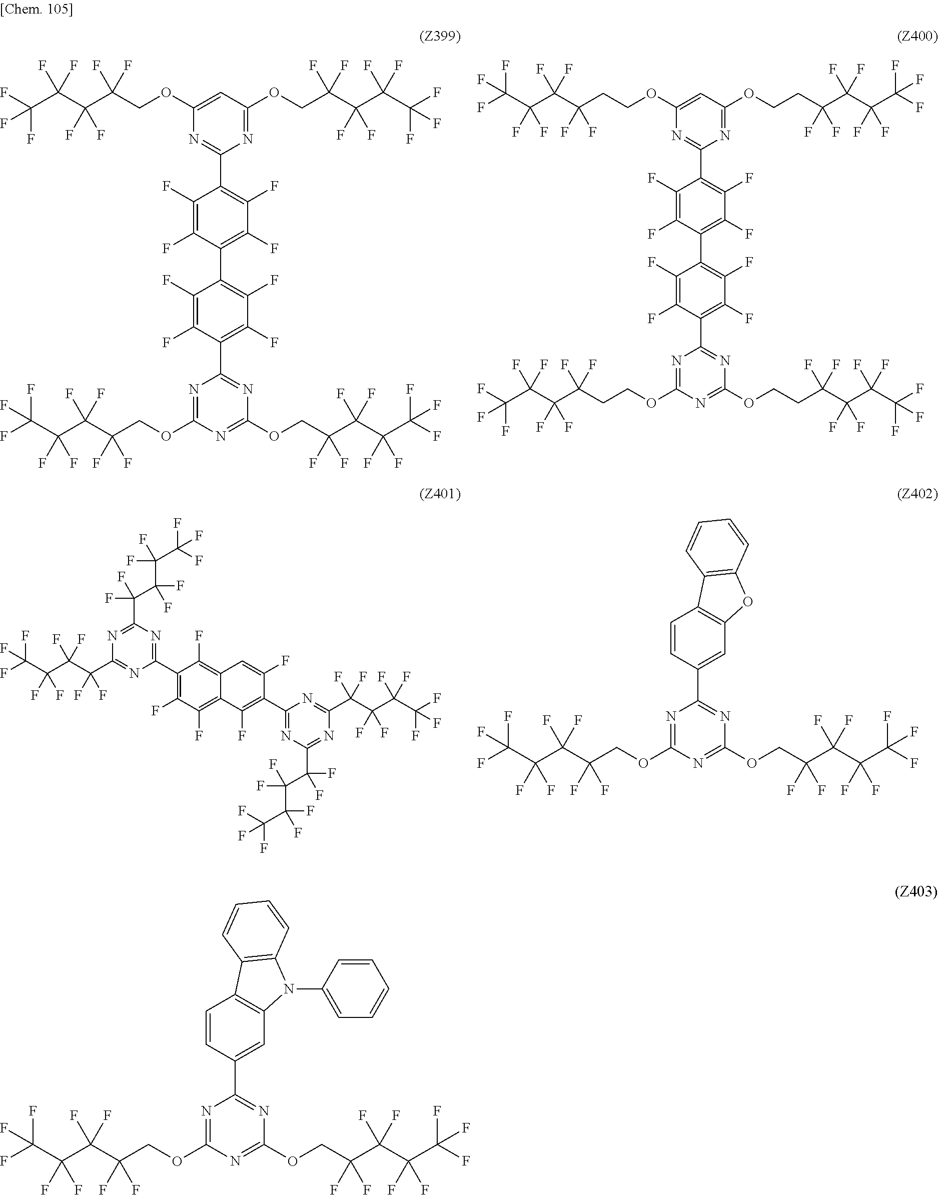

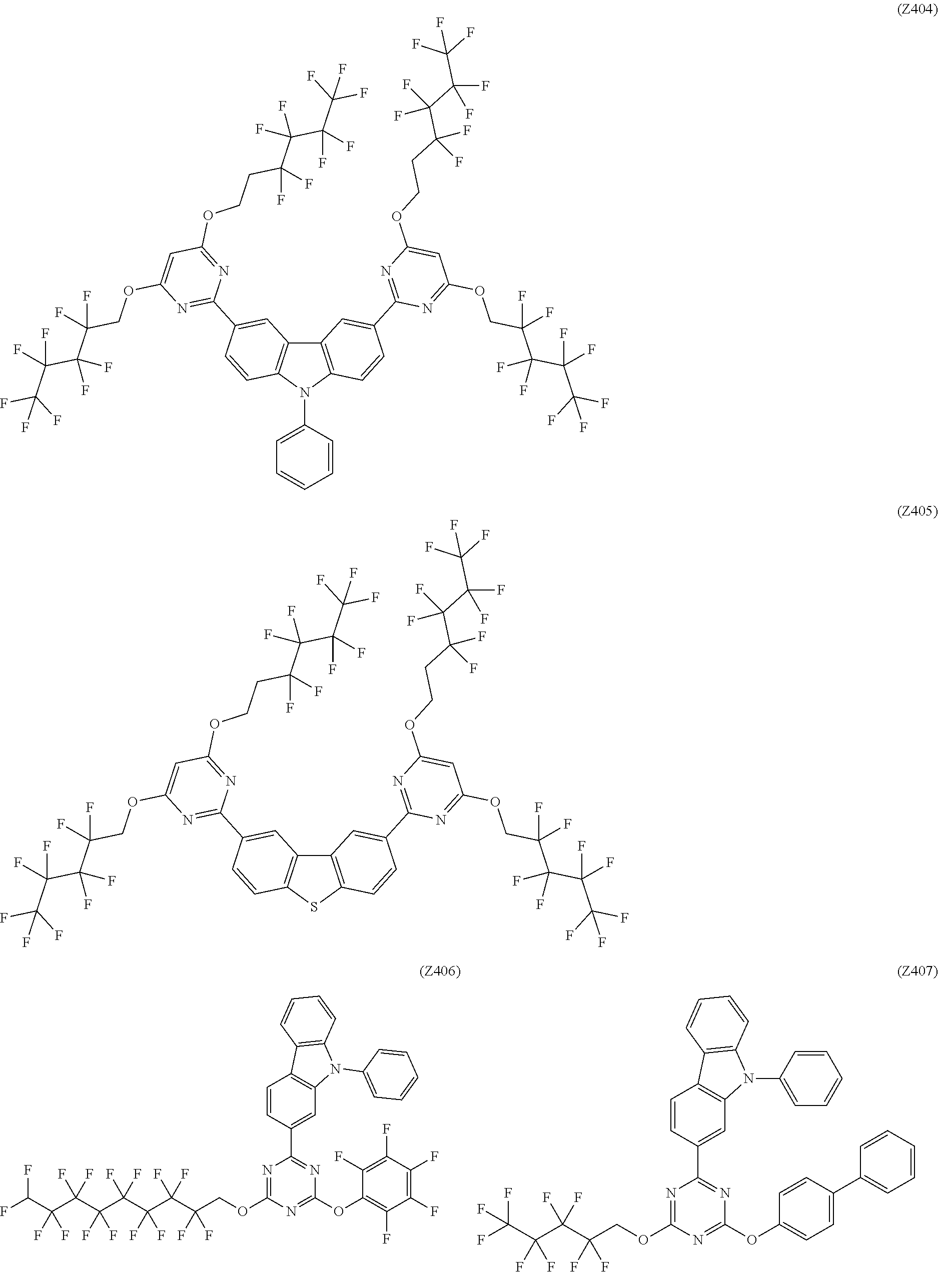

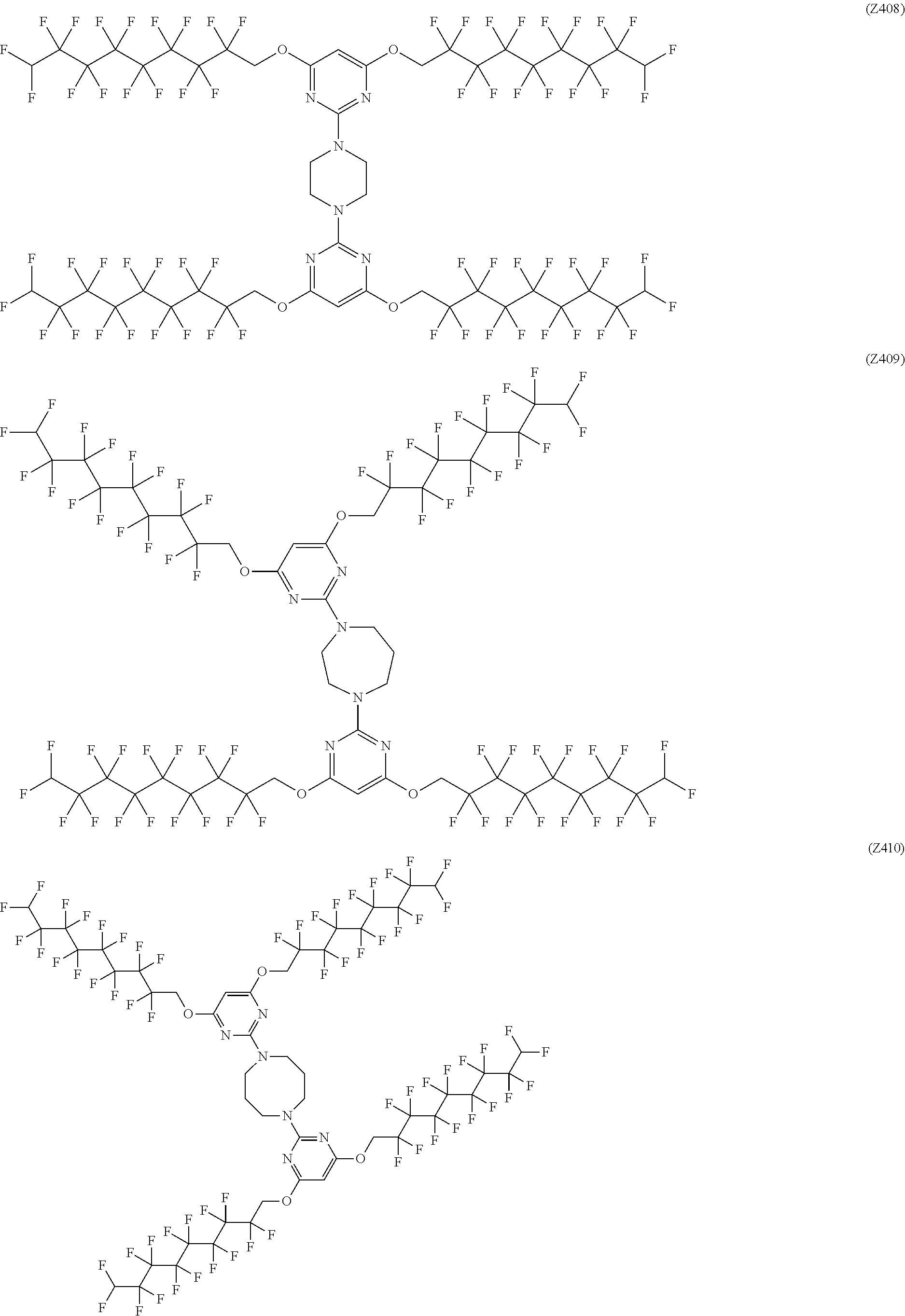

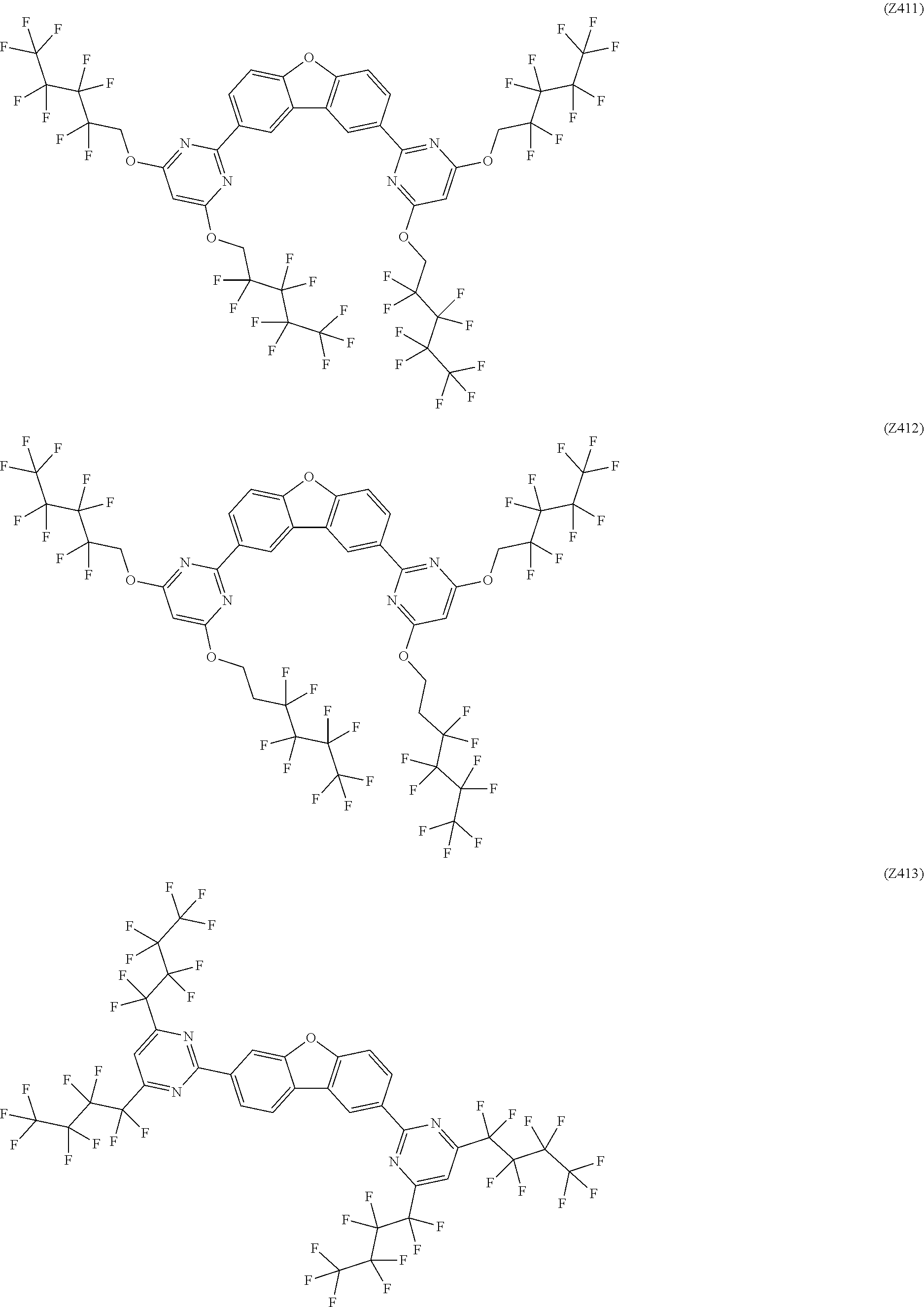

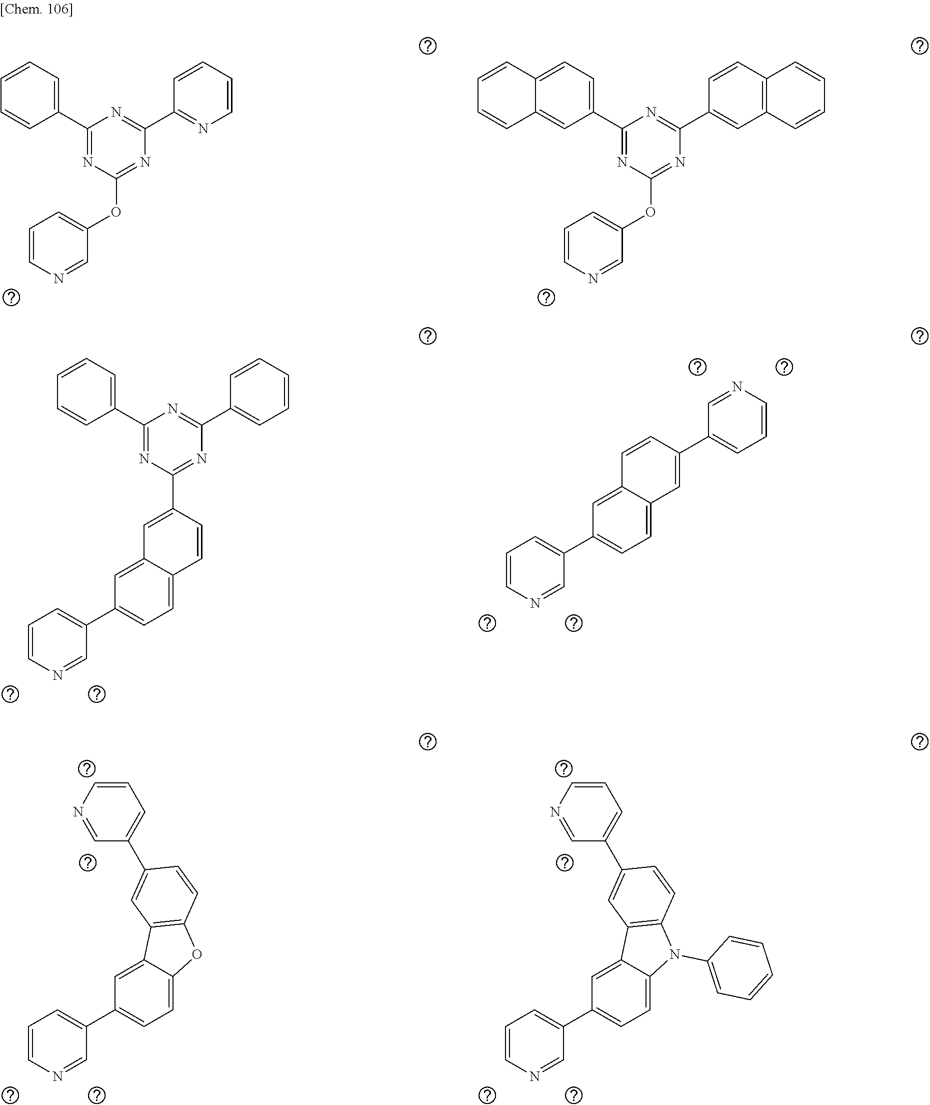 [SMILES: c1ccc(-c2nc(Oc3cccnc3)nc(-c3ccccn3)n2)cc1 ; c1cncc(Oc2nc(-c3ccc4ccccc4c3)nc(-c3ccc4ccccc4c3)n2)c1 ; c1ccc(-c2nc(-c3ccccc3)nc(-c3ccc4ccc(-c5cccnc5)cc4c3)n2)cc1 ; c1cncc(-c2ccc3cc(-c4cccnc4)ccc3c2)c1 ; c1cncc(-c2ccc3oc4ccc(-c5cccnc5)cc4c3c2)c1 ; c1ccc(-n2c3ccc(-c4cccnc4)cc3c3cc(-c4cccnc4)ccc32)cc1]

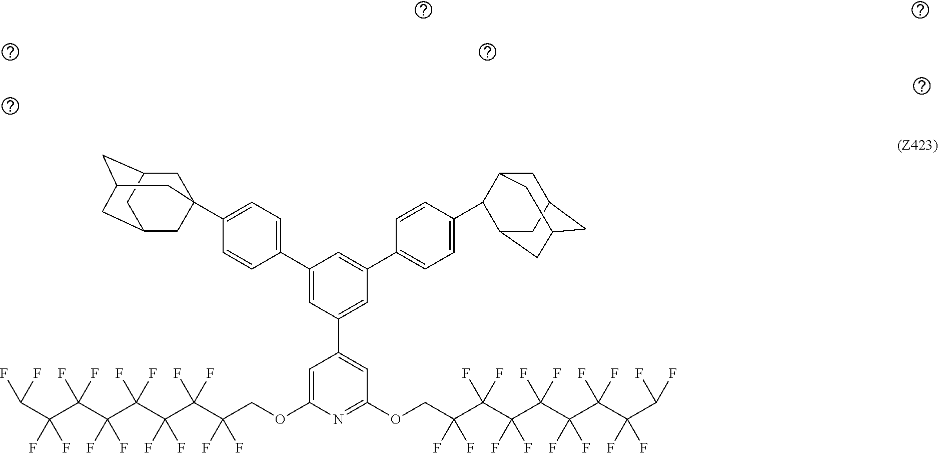 [SMILES: FC(F)C(F)(F)C(F)(F)C(F)(F)C(F)(F)C(F)(F)C(F)(F)C(F)(F)COc1cc(-c2cc(-c3ccc(C4C5CC6CC(C5)CC4C6)cc3)cc(-c3ccc(C45CC6CC(CC(C6)C4)C5)cc3)c2)cc(OCC(F)(F)C(F)(F)C(F)(F)C(F)(F)C(F)(F)C(F)(F)C(F)(F)C(F)F)n1]

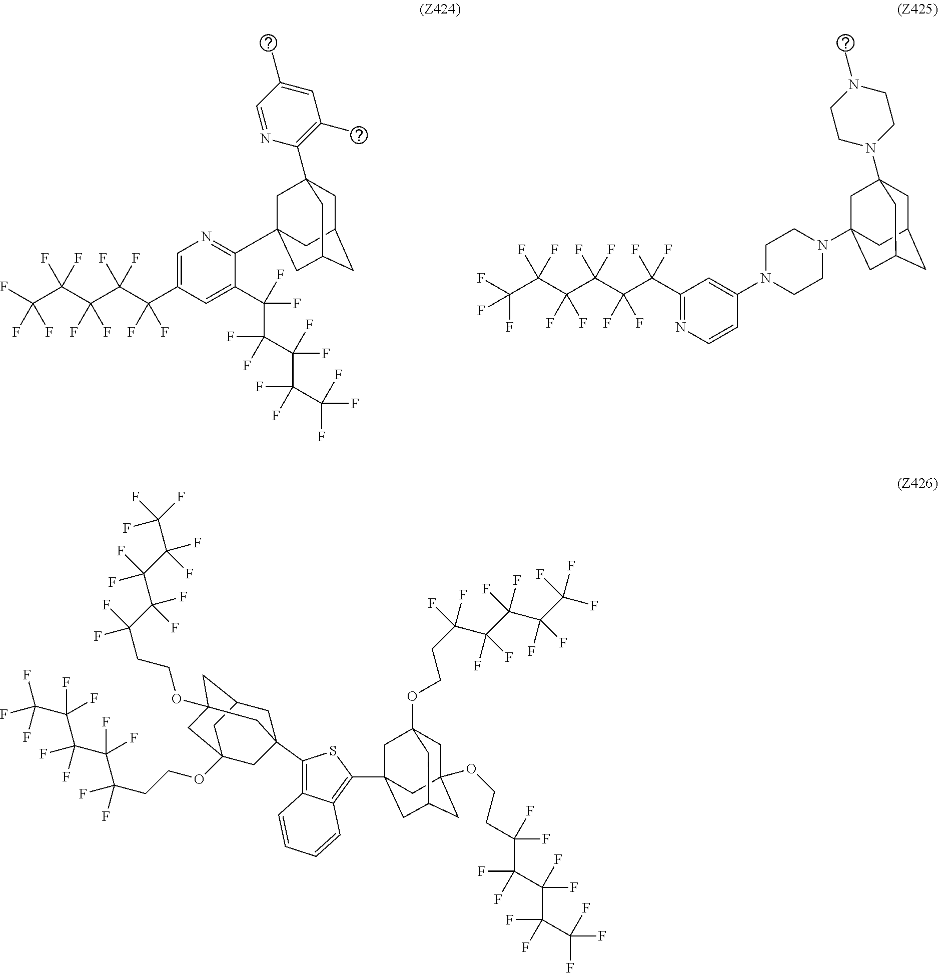 [SMILES: Cc1cnc(C23CC4CC(C2)CC(c2ncc(C(F)(F)C(F)(F)C(F)(F)C(F)(F)C(F)(F)F)cc2C(F)(F)C(F)(F)C(F)(F)C(F)(F)C(F)(F)F)(C4)C3)c(C)c1 ; CN1CCN(C23CC4CC(C2)CC(N2CCN(c5ccnc(C(F)(F)C(F)(F)C(F)(F)C(F)(F)C(F)(F)C(F)(F)F)c5)CC2)(C4)C3)CC1 ; FC(F)(F)C(F)(F)C(F)(F)C(F)(F)C(F)(F)CCOC12CC3CC(OCCC(F)(F)C(F)(F)C(F)(F)C(F)(F)C(F)(F)F)(C1)CC(c1sc(C45CC6CC(OCCC(F)(F)C(F)(F)C(F)(F)C(F)(F)C(F)(F)F)(CC(OCCC(F)(F)C(F)(F)C(F)(F)C(F)(F)C(F)(F)F)(C6)C4)C5)c4ccccc14)(C3)C2]

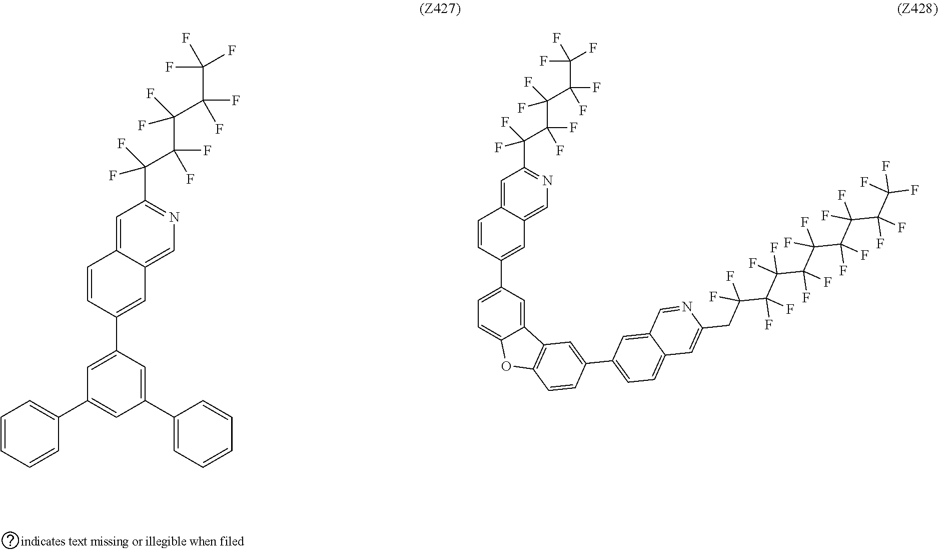 [SMILES: FC(F)(F)C(F)(F)C(F)(F)C(F)(F)C(F)(F)c1cc2ccc(-c3cc(-c4ccccc4)cc(-c4ccccc4)c3)cc2cn1 ; FC(F)(F)C(F)(F)C(F)(F)C(F)(F)C(F)(F)c1cc2ccc(-c3ccc4oc5ccc(-c6ccc7cc(CC(F)(F)C(F)(F)C(F)(F)C(F)(F)C(F)(F)C(F)(F)C(F)(F)C(F)(F)C(F)(F)F)ncc7c6)cc5c4c3)cc2cn1]

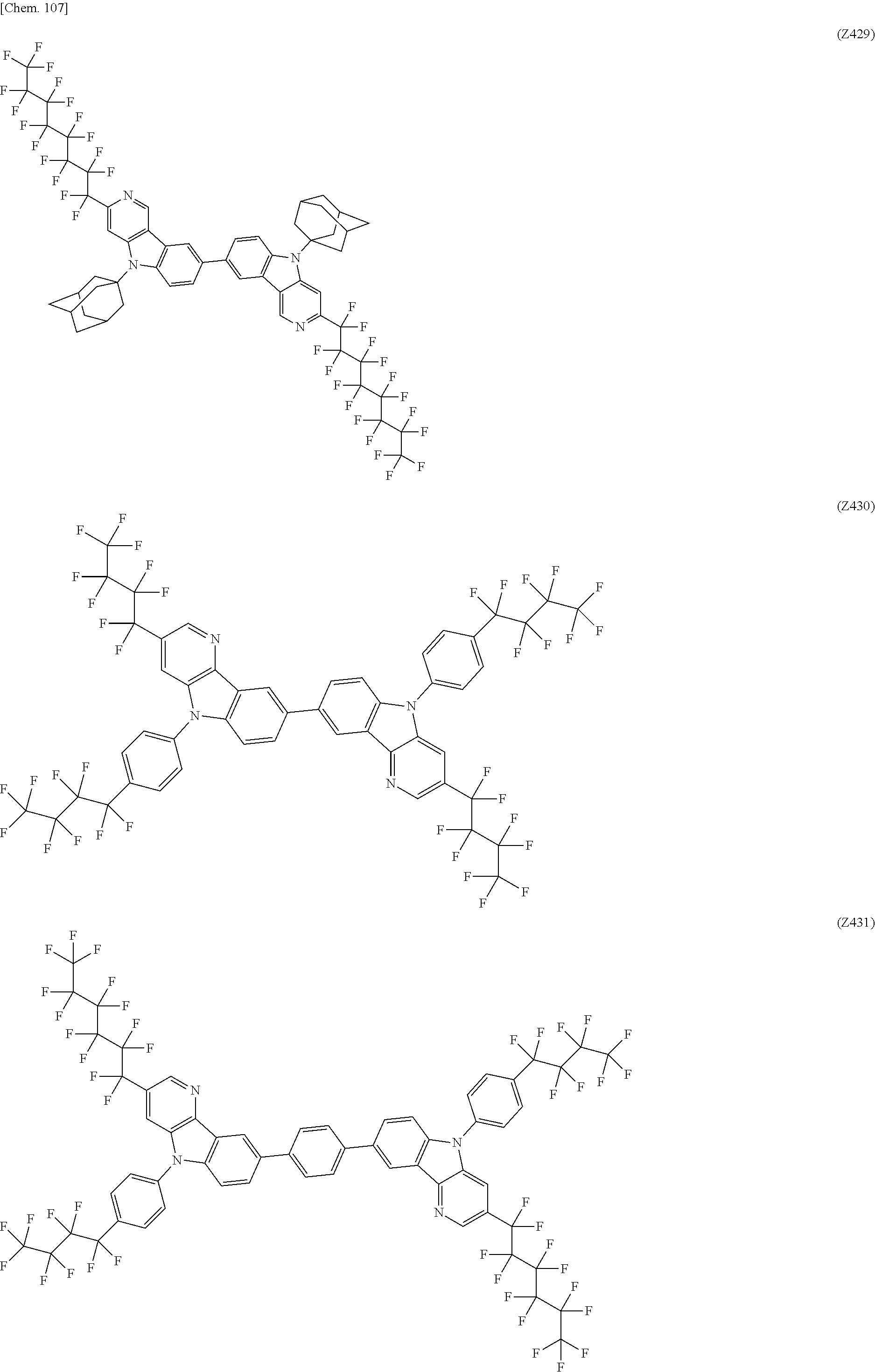

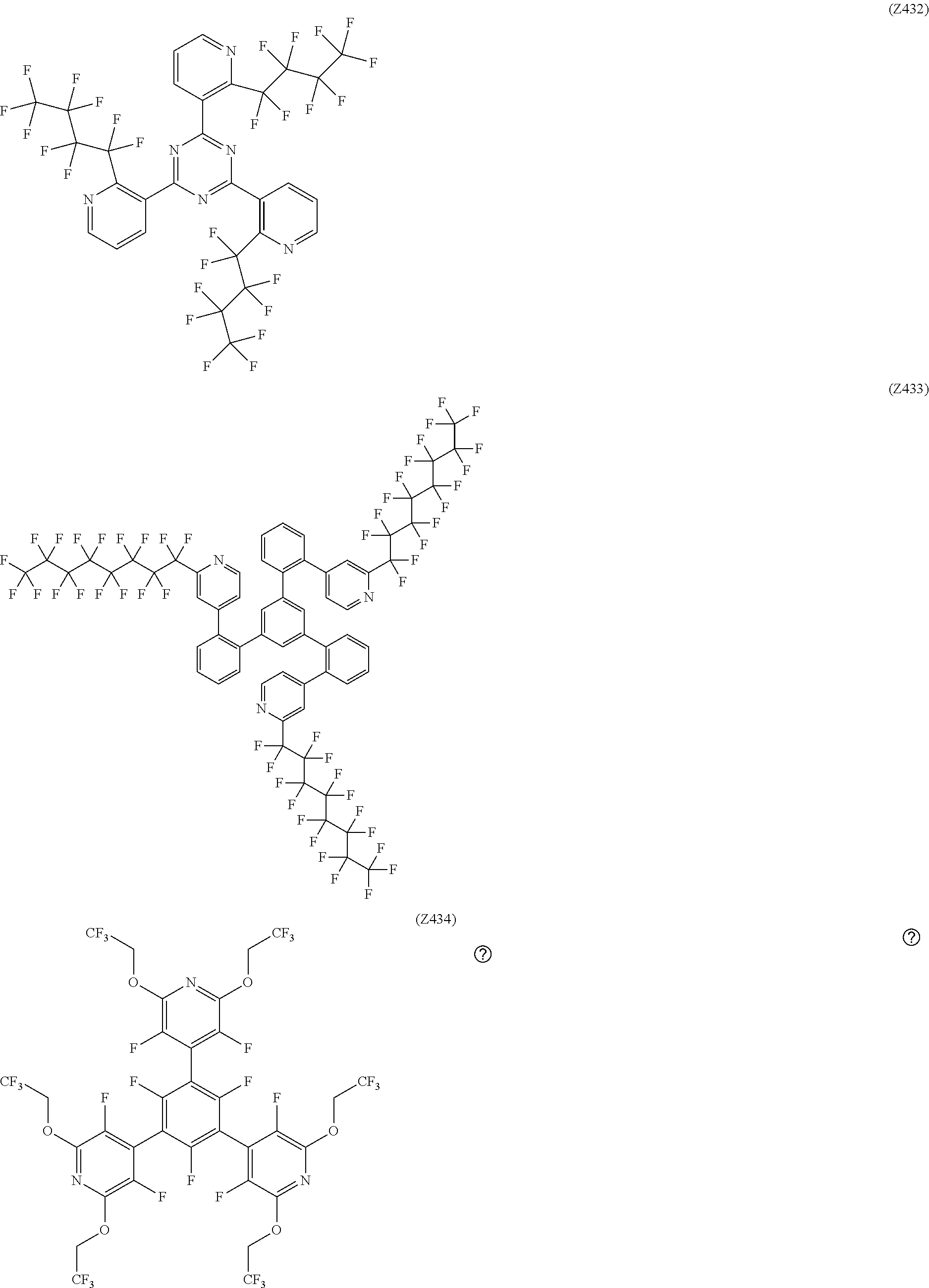

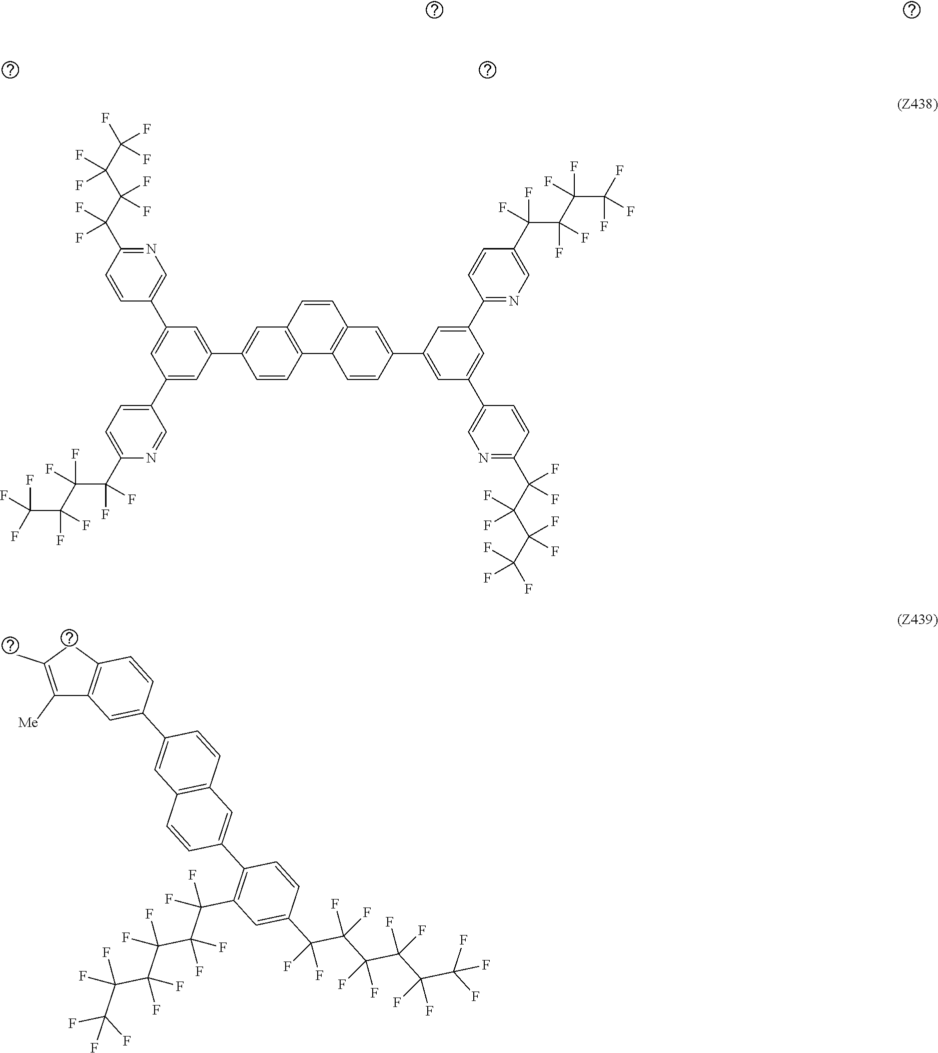 [SMILES: FC(F)(F)C(F)(F)C(F)(F)C(F)(F)c1ccc(-c2cc(-c3ccc(C(F)(F)C(F)(F)C(F)(F)C(F)(F)F)nc3)cc(-c3ccc4c(ccc5cc(-c6cc(-c7ccc(C(F)(F)C(F)(F)C(F)(F)C(F)(F)F)nc7)cc(-c7ccc(C(F)(F)C(F)(F)C(F)(F)C(F)(F)F)nc7)c6)ccc54)c3)c2)nc1 ; CC1=C(C)c2cc(-c3ccc4cc(-c5ccc(C(F)(F)C(F)(F)C(F)(F)C(F)(F)C(F)(F)C(F)(F)F)cc5C(F)(F)C(F)(F)C(F)(F)C(F)(F)C(F)(F)C(F)(F)F)ccc4c3)ccc2C1]

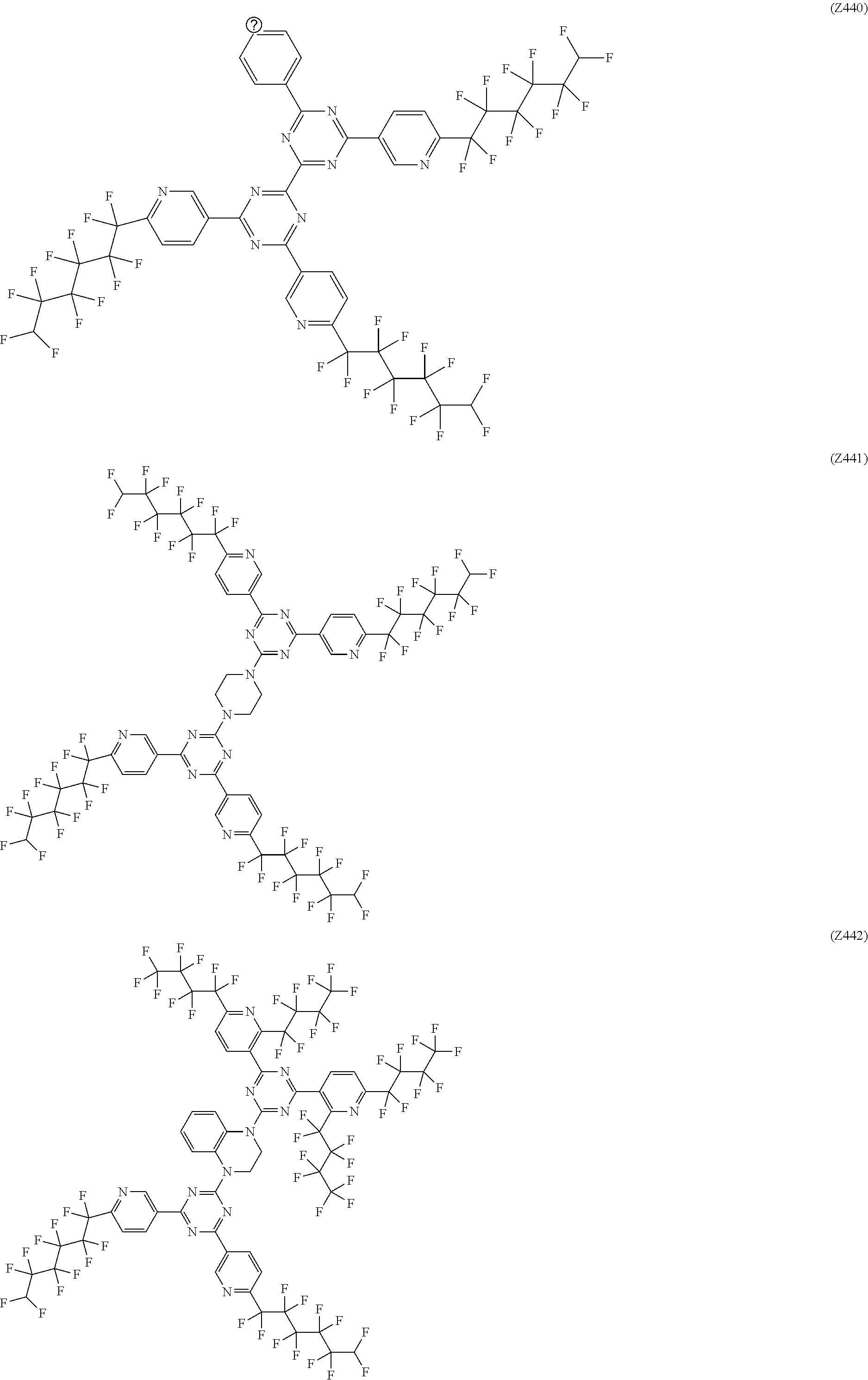

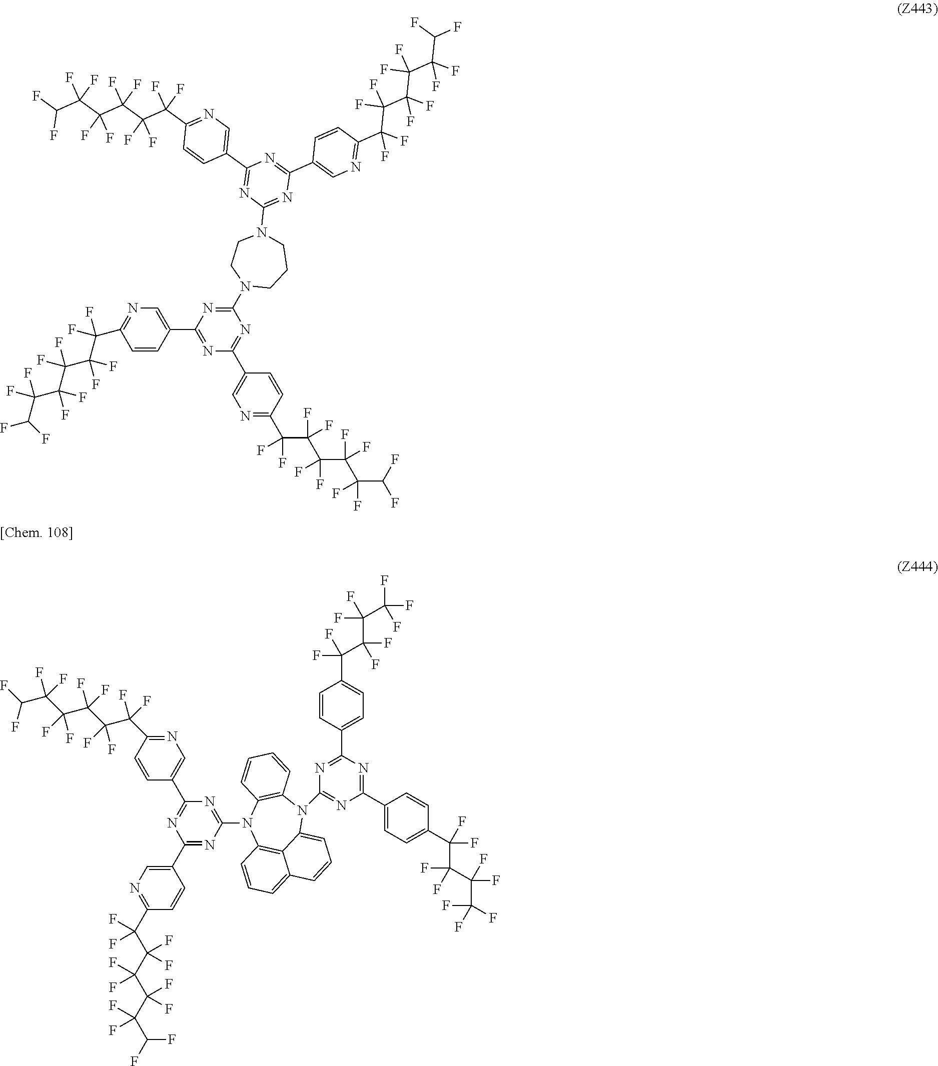 [SMILES: FC(F)C(F)(F)C(F)(F)C(F)(F)C(F)(F)C(F)(F)c1ccc(-c2nc(-c3ccc(C(F)(F)C(F)(F)C(F)(F)C(F)(F)C(F)(F)C(F)F)nc3)nc(N3CCCN(c4nc(-c5ccc(C(F)(F)C(F)(F)C(F)(F)C(F)(F)C(F)(F)C(F)F)nc5)nc(-c5ccc(C(F)(F)C(F)(F)C(F)(F)C(F)(F)C(F)(F)C(F)F)nc5)n4)CC3)n2)cn1 ; FC(F)C(F)(F)C(F)(F)C(F)(F)C(F)(F)C(F)(F)c1ccc(-c2nc(-c3ccc(C(F)(F)C(F)(F)C(F)(F)C(F)(F)C(F)(F)C(F)F)nc3)nc(N3c4ccccc4N(c4nc(-c5ccc(C(F)(F)C(F)(F)C(F)(F)C(F)(F)F)cc5)nc(-c5ccc(C(F)(F)C(F)(F)C(F)(F)C(F)(F)F)cc5)n4)c4cccc5cccc3c45)n2)cn1]

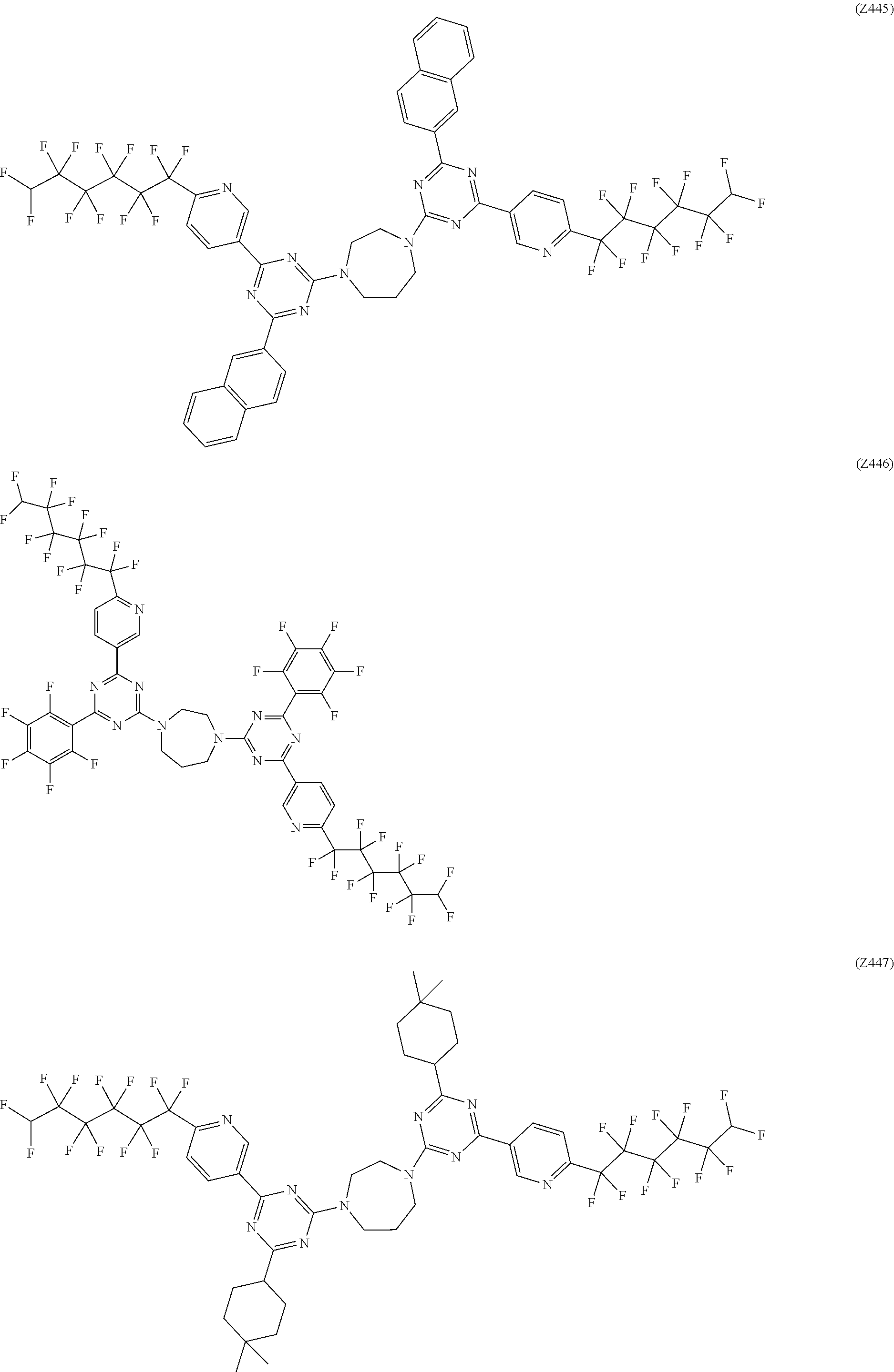

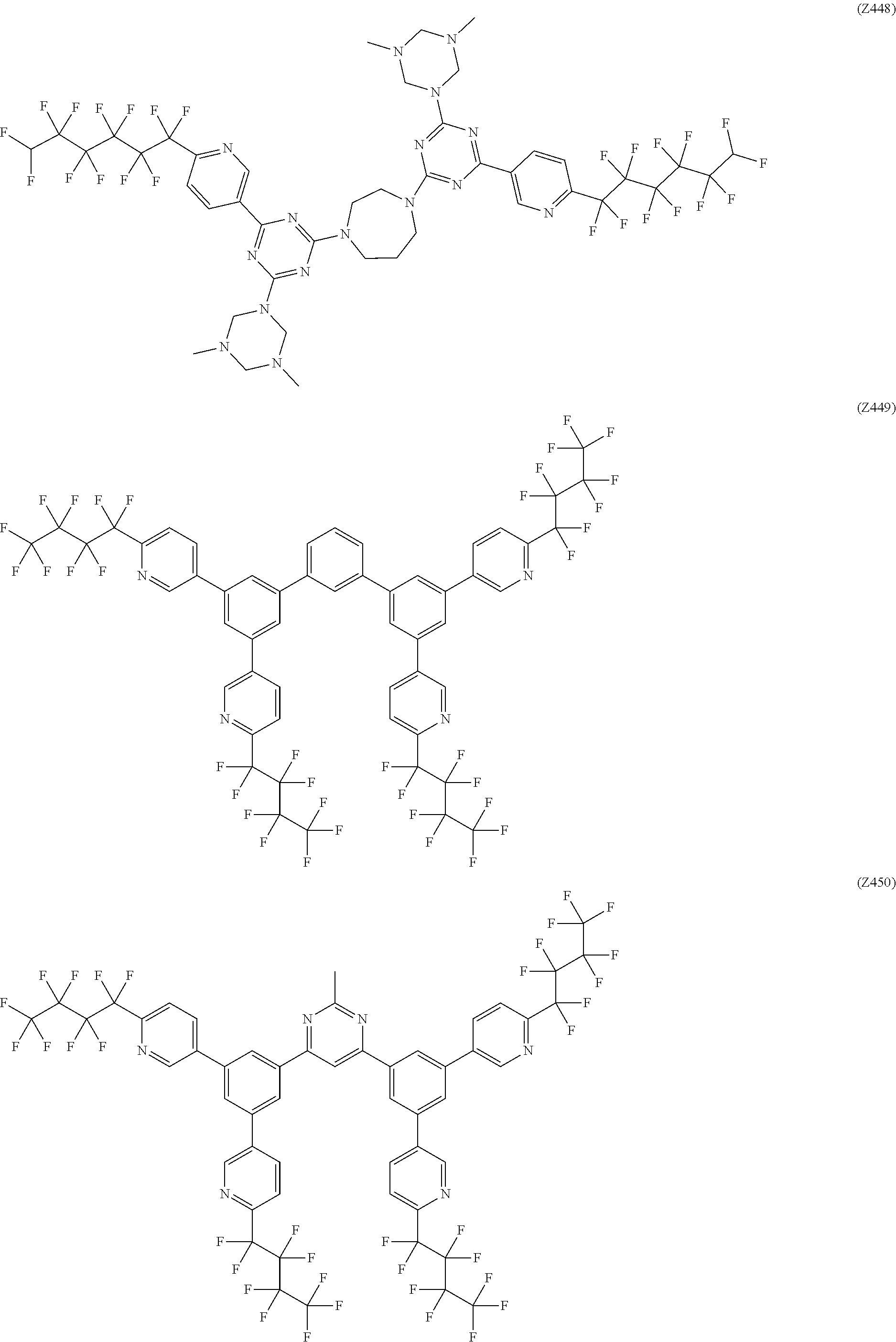

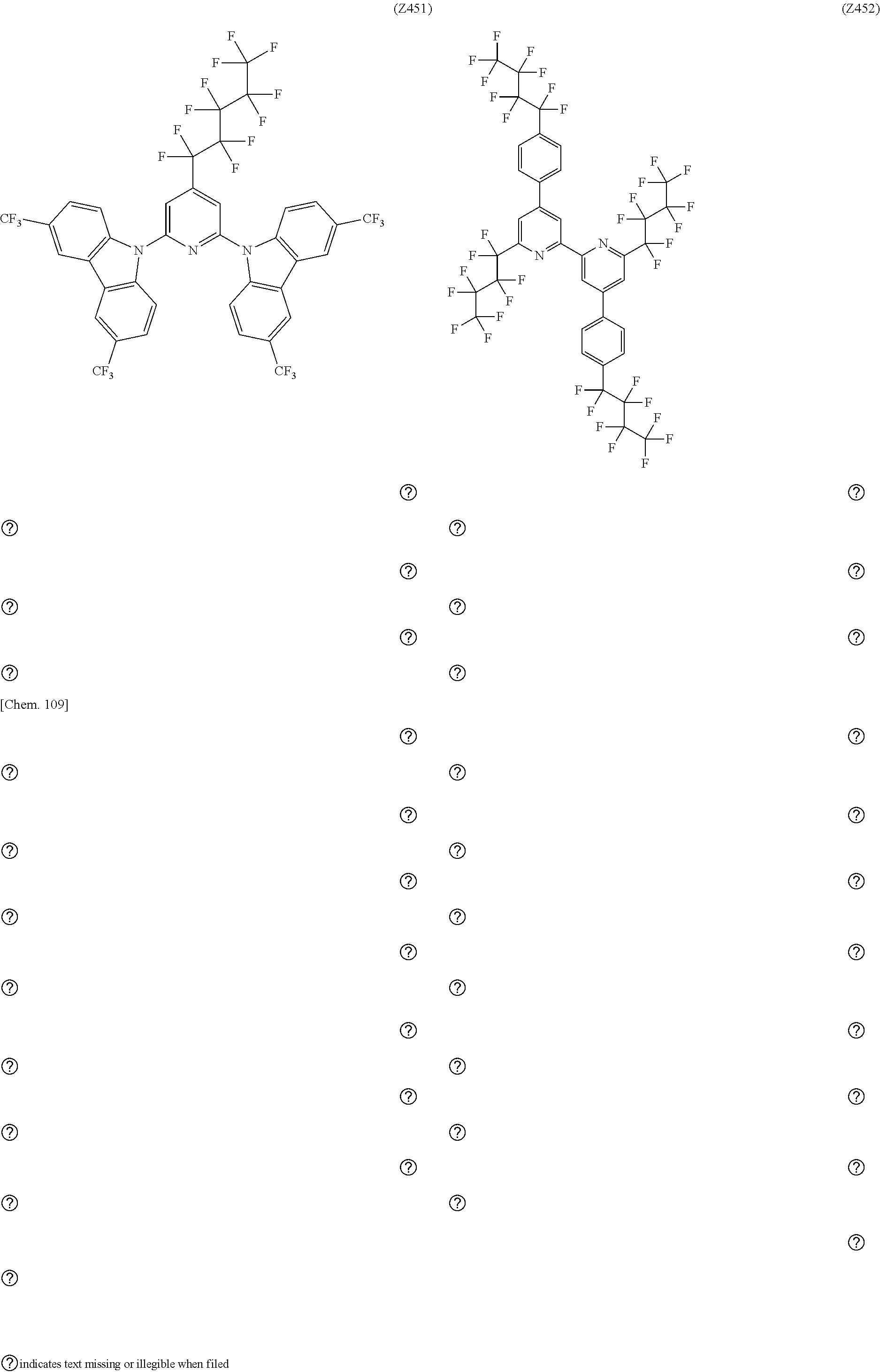 [SMILES: FC(F)(F)c1ccc2c(c1)c1cc(C(F)(F)F)ccc1n2-c1cc(C(F)(F)C(F)(F)C(F)(F)C(F)(F)C(F)(F)F)cc(-n2c3ccc(C(F)(F)F)cc3c3cc(C(F)(F)F)ccc32)n1 ; FC(F)(F)C(F)(F)C(F)(F)C(F)(F)c1ccc(-c2cc(-c3cc(-c4ccc(C(F)(F)C(F)(F)C(F)(F)C(F)(F)F)cc4)cc(C(F)(F)C(F)(F)C(F)(F)C(F)(F)F)n3)nc(C(F)(F)C(F)(F)C(F)(F)C(F)(F)F)c2)cc1]

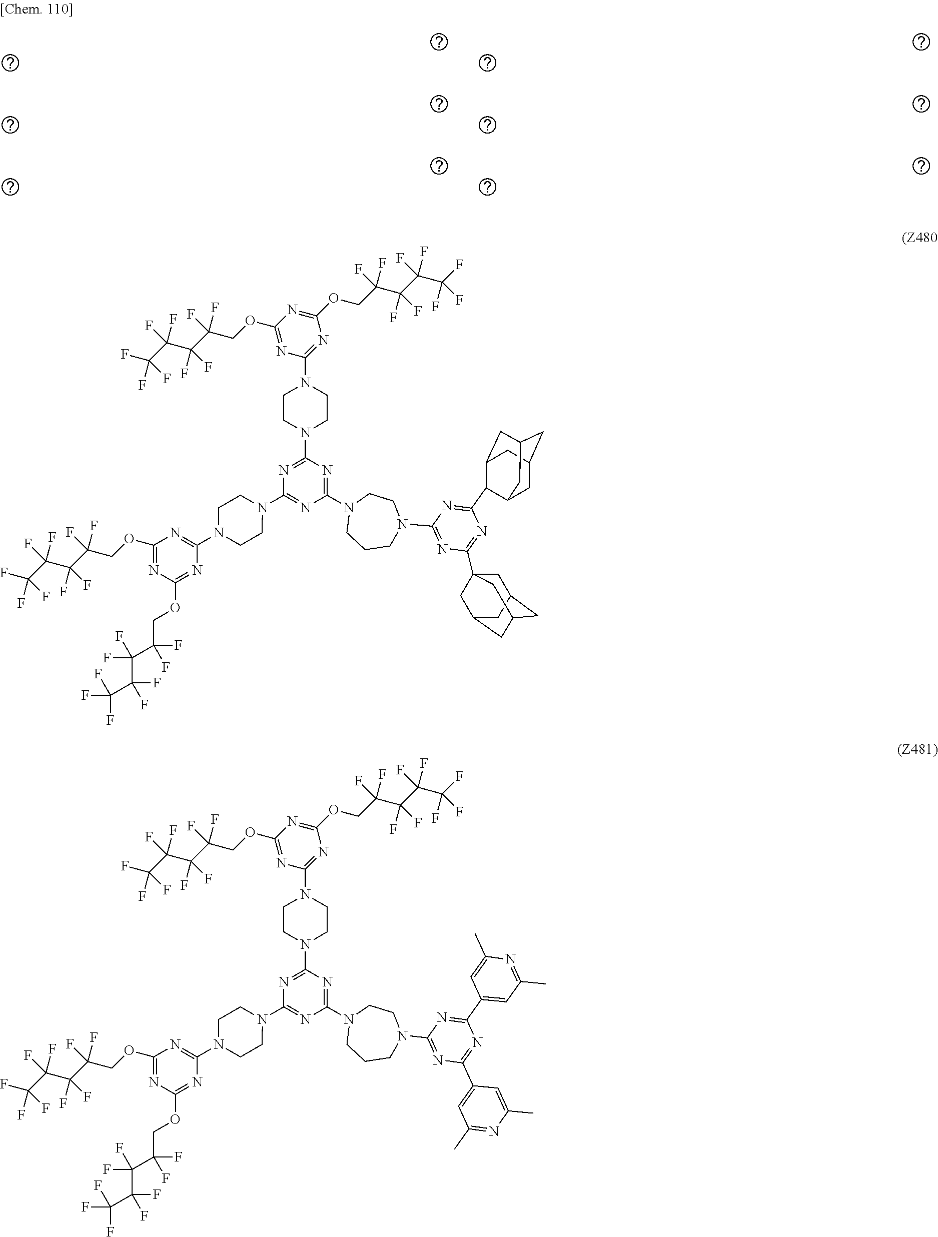 [SMILES: FC(F)(F)C(F)(F)C(F)(F)C(F)(F)COc1nc(OCC(F)(F)C(F)(F)C(F)(F)C(F)(F)F)nc(N2CCN(c3nc(N4CCN(c5nc(OCC(F)(F)C(F)(F)C(F)(F)C(F)(F)F)nc(OCC(F)(F)C(F)(F)C(F)(F)C(F)(F)F)n5)CC4)nc(N4CCCN(c5nc(C6C7CC8CC(C7)CC6C8)nc(C67CC8CC(CC(C8)C6)C7)n5)CC4)n3)CC2)n1 ; Cc1cc(-c2nc(-c3cc(C)nc(C)c3)nc(N3CCCN(c4nc(N5CCN(c6nc(OCC(F)(F)C(F)(F)C(F)(F)C(F)(F)F)nc(OCC(F)(F)C(F)(F)C(F)(F)C(F)(F)F)n6)CC5)nc(N5CCN(c6nc(OCC(F)(F)C(F)(F)C(F)(F)C(F)(F)F)nc(OCC(F)(F)C(F)(F)C(F)(F)C(F)(F)F)n6)CC5)n4)CC3)n2)cc(C)n1]

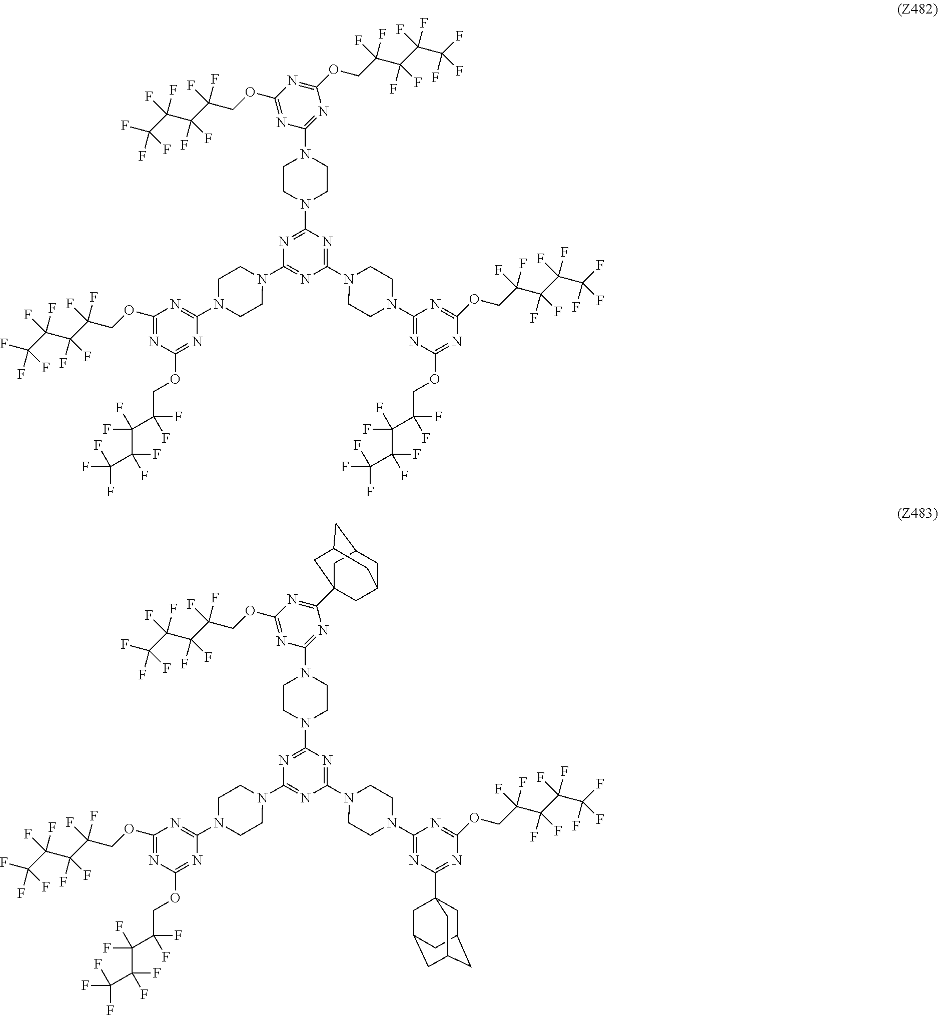

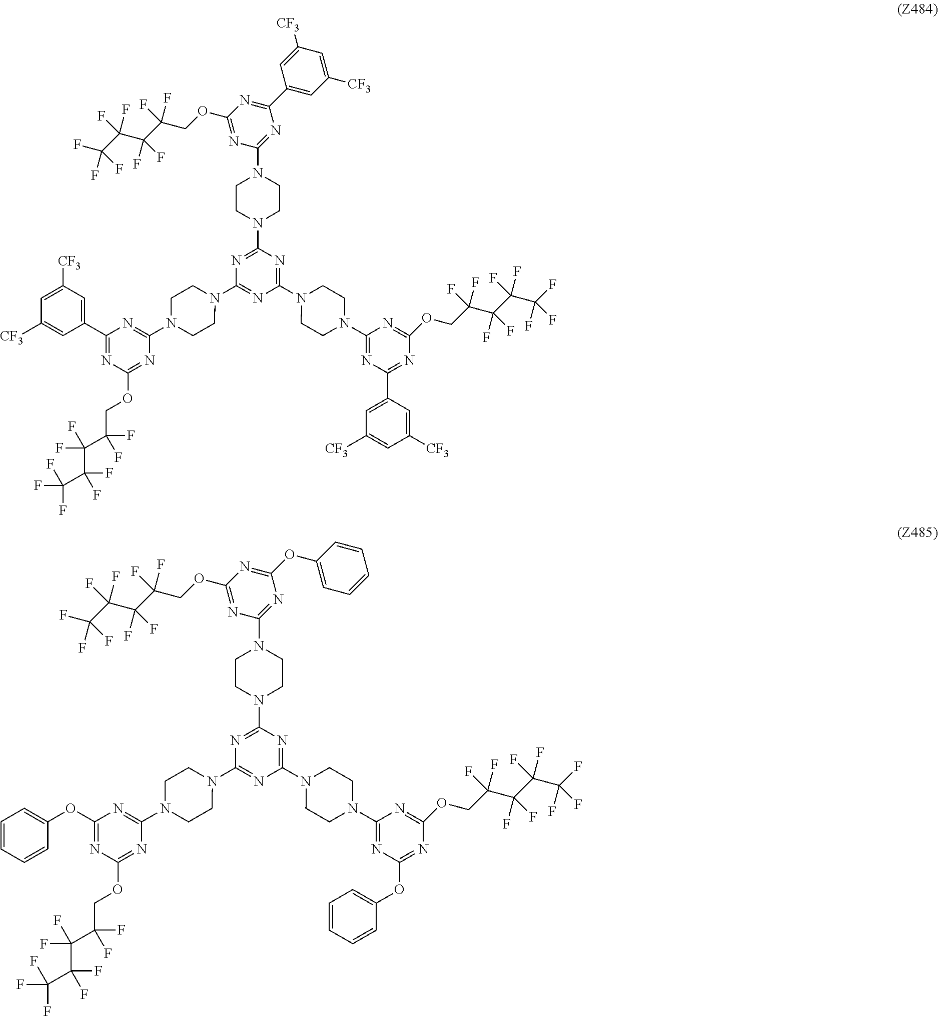 [SMILES: FC(F)(F)c1cc(-c2nc(OCC(F)(F)C(F)(F)C(F)(F)C(F)(F)F)nc(N3CCN(c4nc(N5CCN(c6nc(OCC(F)(F)C(F)(F)C(F)(F)C(F)(F)F)nc(-c7cc(C(F)(F)F)cc(C(F)(F)F)c7)n6)CC5)nc(N5CCN(c6nc(OCC(F)(F)C(F)(F)C(F)(F)C(F)(F)F)nc(-c7cc(C(F)(F)F)cc(C(F)(F)F)c7)n6)CC5)n4)CC3)n2)cc(C(F)(F)F)c1 ; FC(F)(F)C(F)(F)C(F)(F)C(F)(F)COc1nc(Oc2ccccc2)nc(N2CCN(c3nc(N4CCN(c5nc(OCC(F)(F)C(F)(F)C(F)(F)C(F)(F)F)nc(Oc6ccccc6)n5)CC4)nc(N4CCN(c5nc(OCC(F)(F)C(F)(F)C(F)(F)C(F)(F)F)nc(Oc6ccccc6)n5)CC4)n3)CC2)n1]

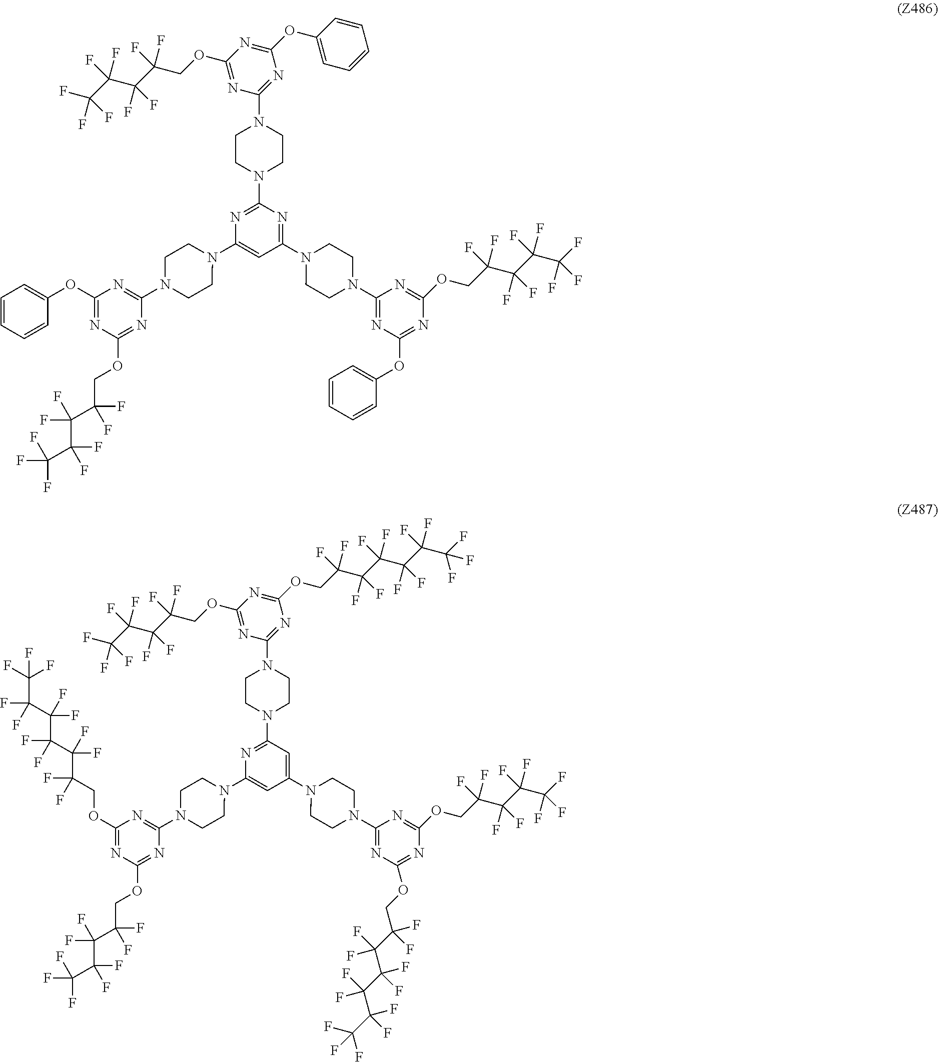

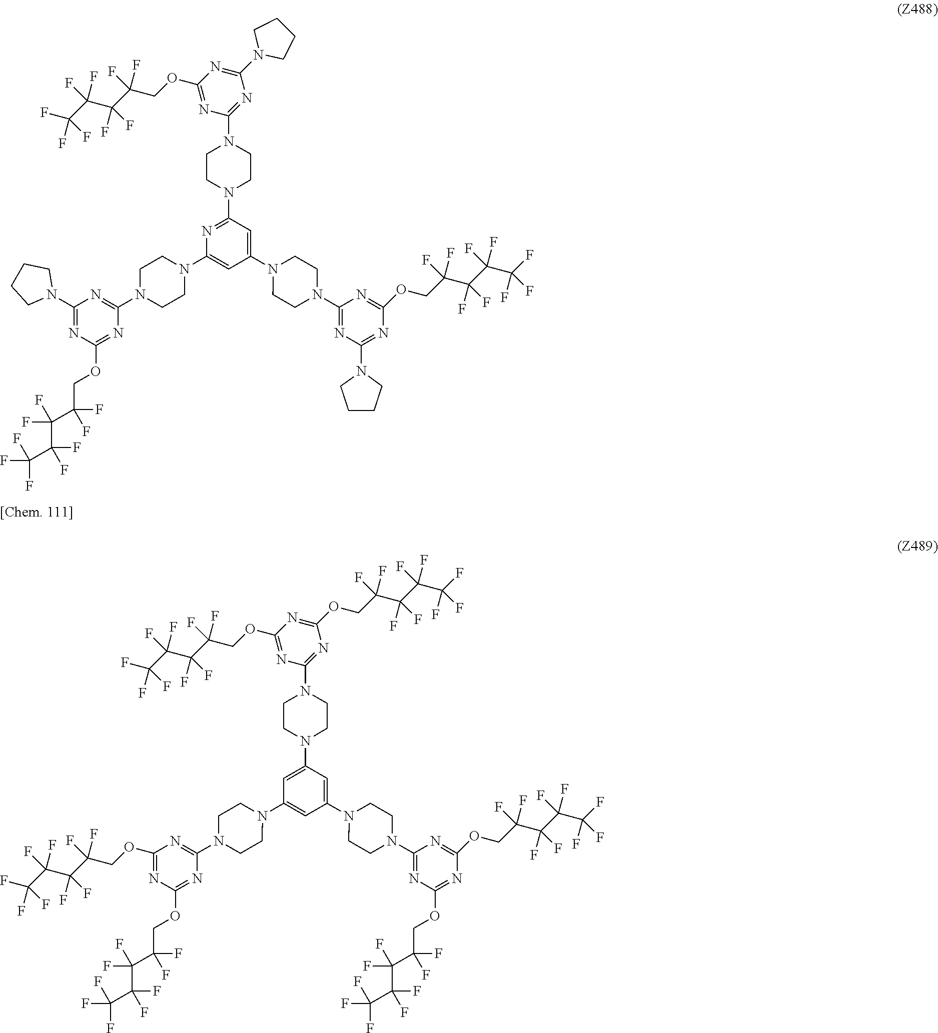 [SMILES: FC(F)(F)C(F)(F)C(F)(F)C(F)(F)COc1nc(N2CCCC2)nc(N2CCN(c3cc(N4CCN(c5nc(OCC(F)(F)C(F)(F)C(F)(F)C(F)(F)F)nc(N6CCCC6)n5)CC4)nc(N4CCN(c5nc(OCC(F)(F)C(F)(F)C(F)(F)C(F)(F)F)nc(N6CCCC6)n5)CC4)c3)CC2)n1 ; FC(F)(F)C(F)(F)C(F)(F)C(F)(F)COc1nc(OCC(F)(F)C(F)(F)C(F)(F)C(F)(F)F)nc(N2CCN(c3cc(N4CCN(c5nc(OCC(F)(F)C(F)(F)C(F)(F)C(F)(F)F)nc(OCC(F)(F)C(F)(F)C(F)(F)C(F)(F)F)n5)CC4)cc(N4CCN(c5nc(OCC(F)(F)C(F)(F)C(F)(F)C(F)(F)F)nc(OCC(F)(F)C(F)(F)C(F)(F)C(F)(F)F)n5)CC4)c3)CC2)n1]

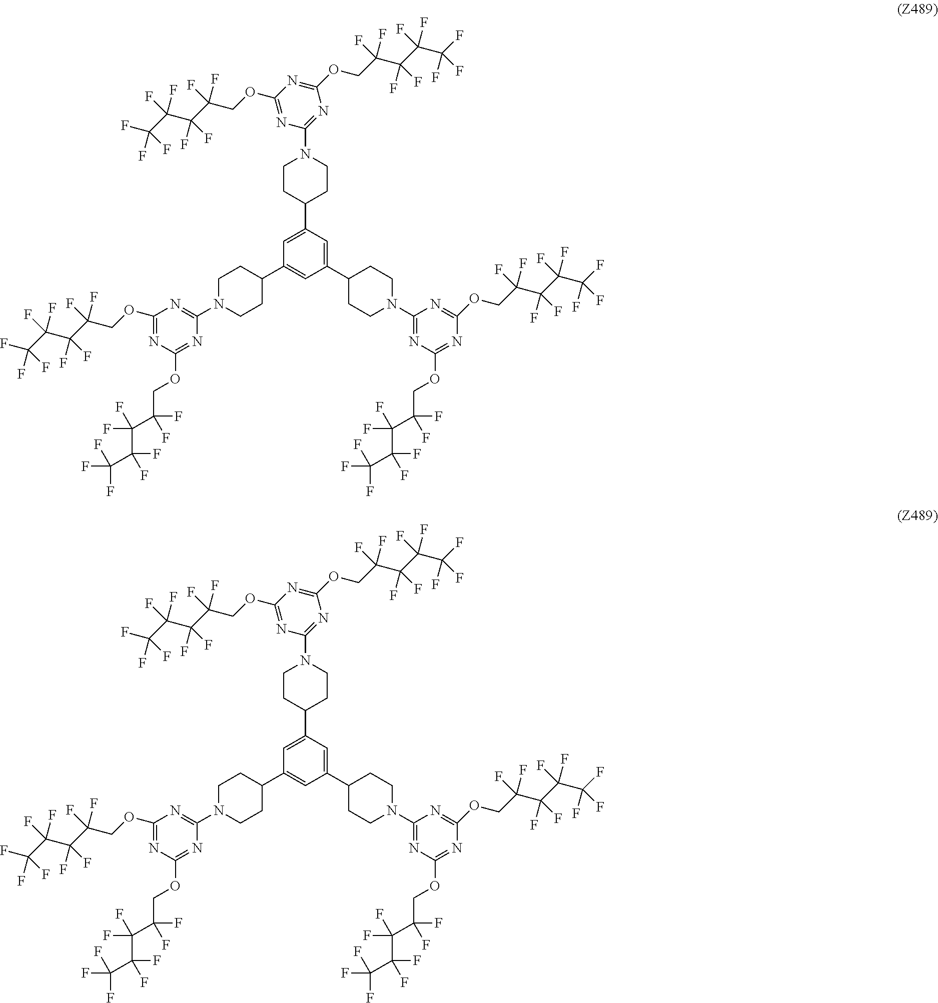

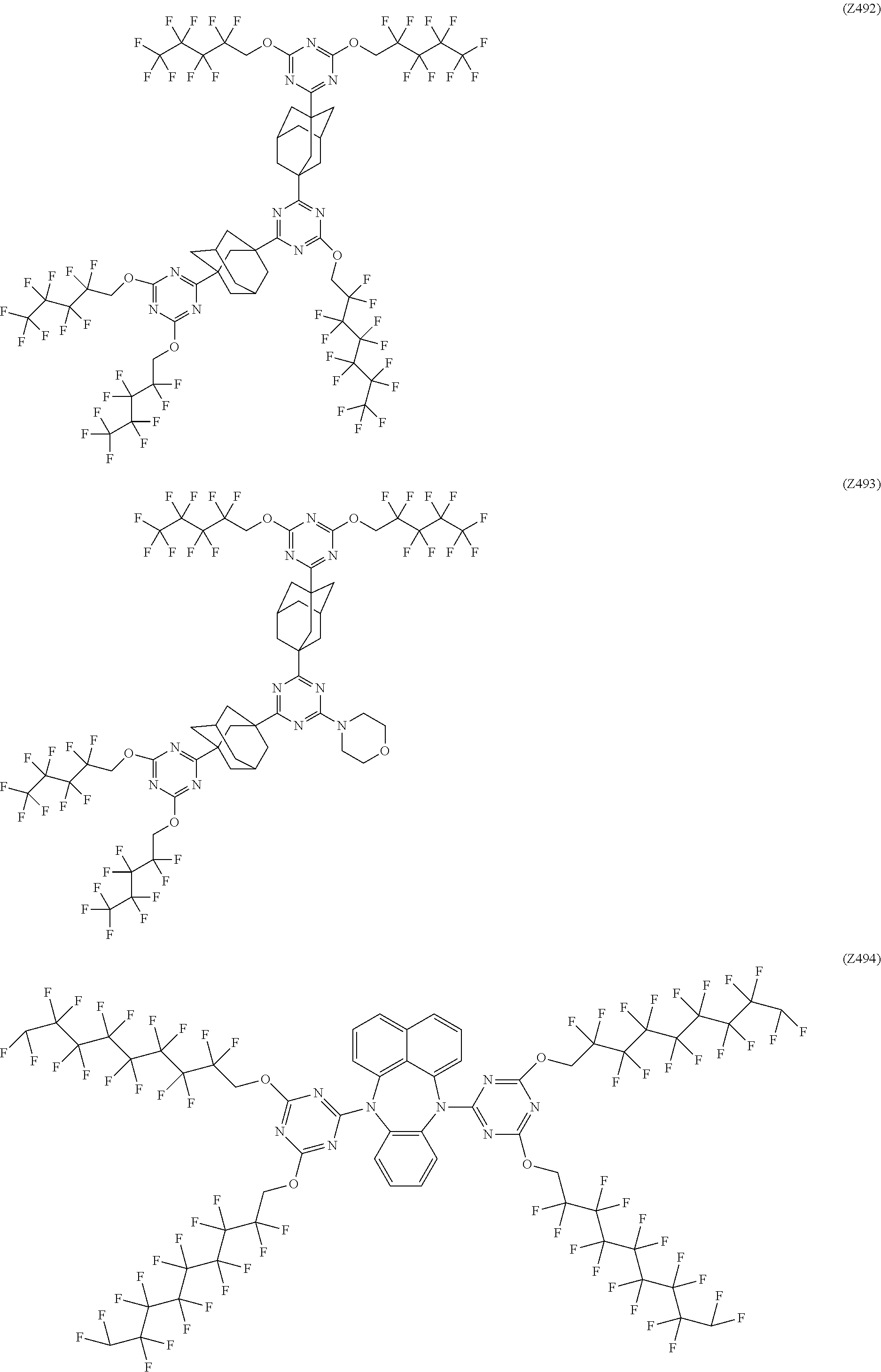

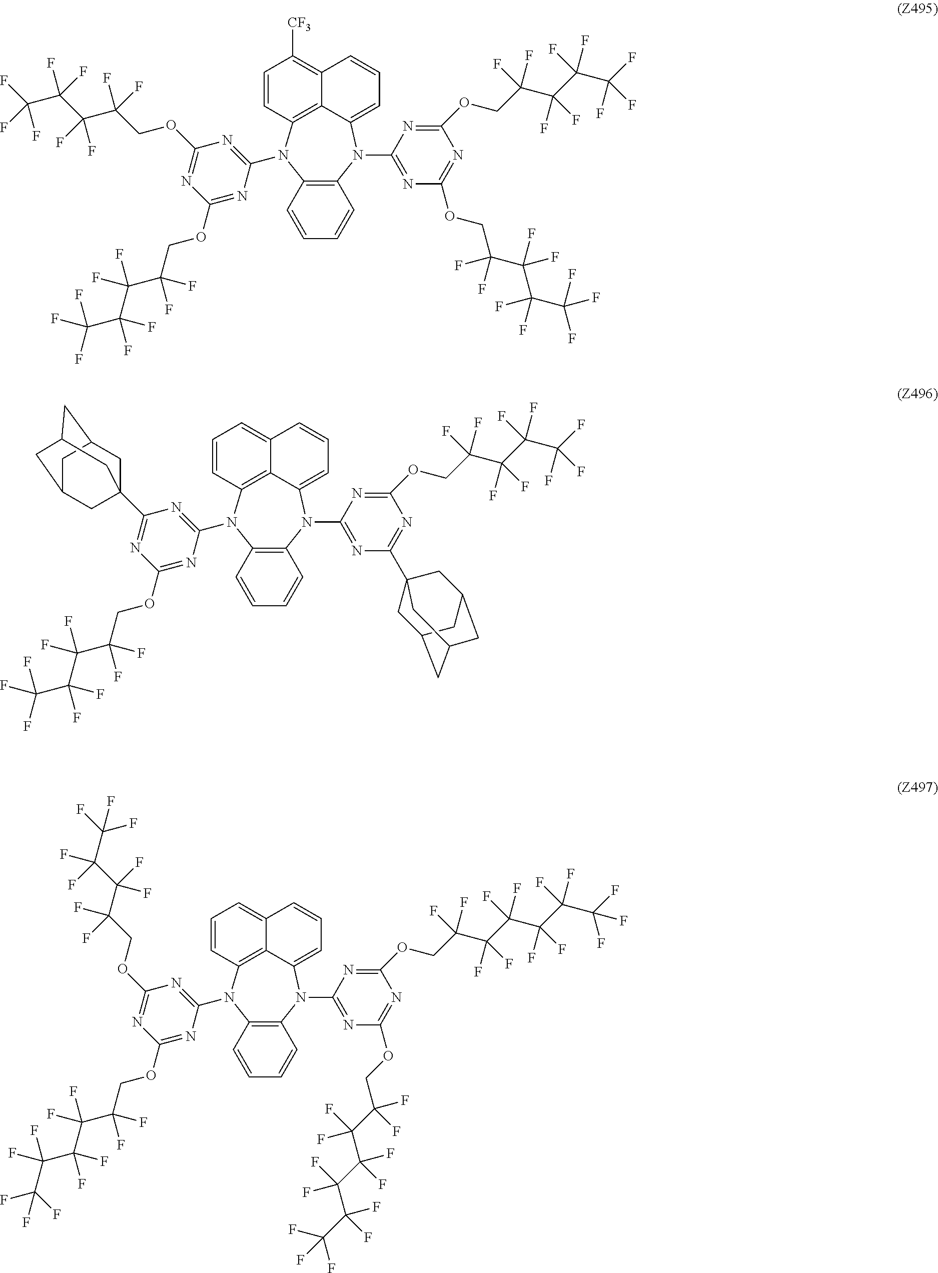

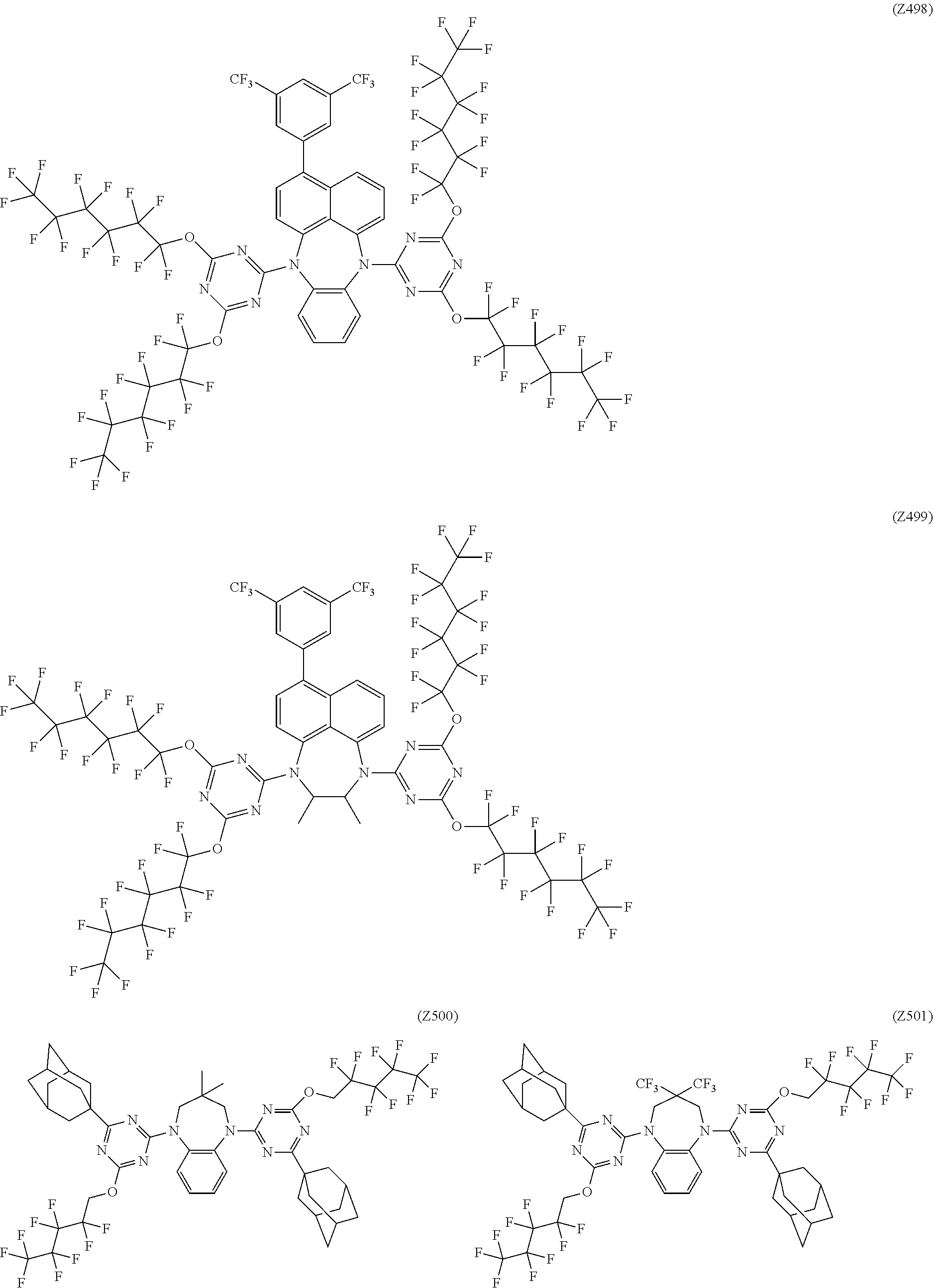

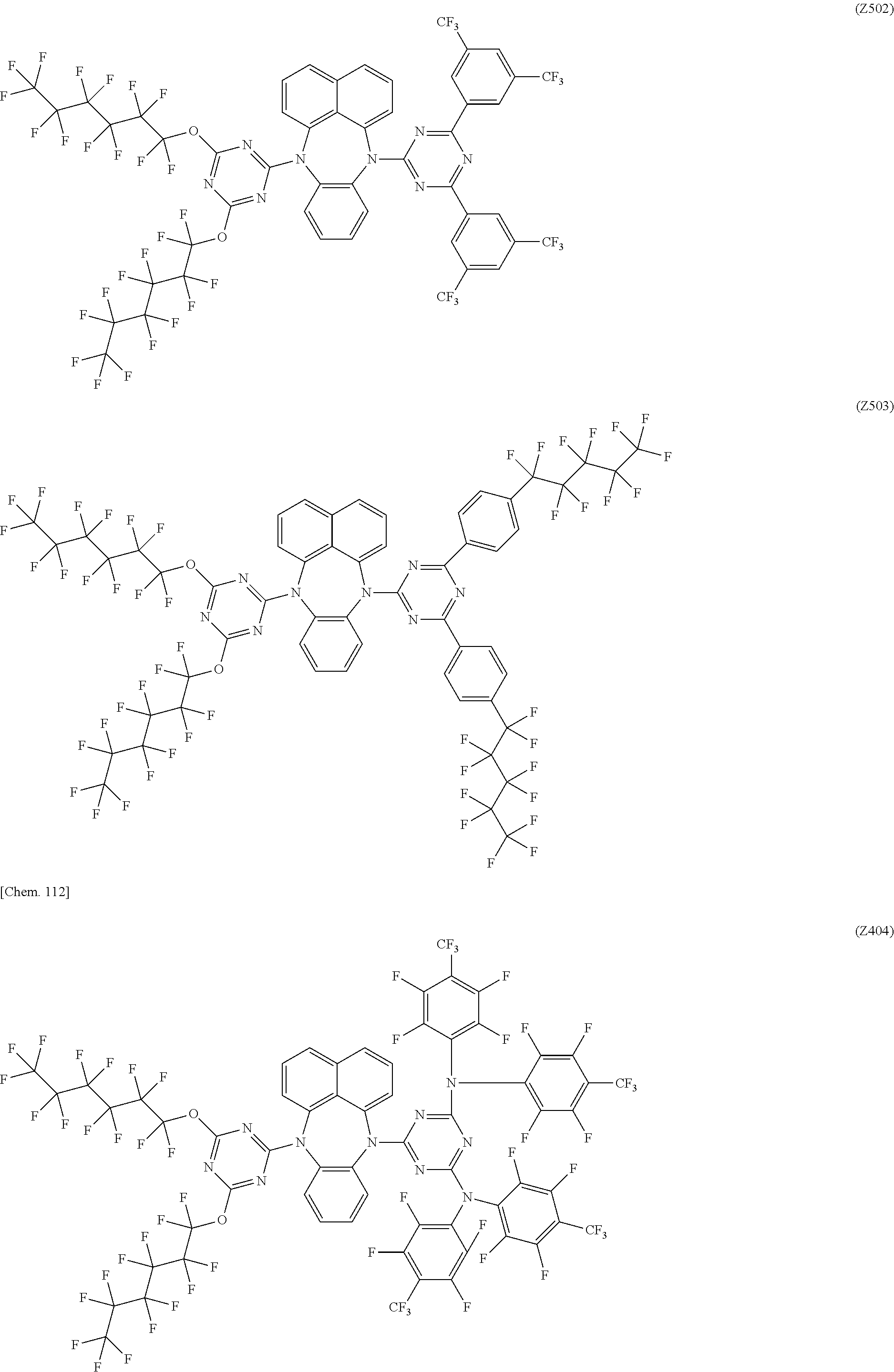

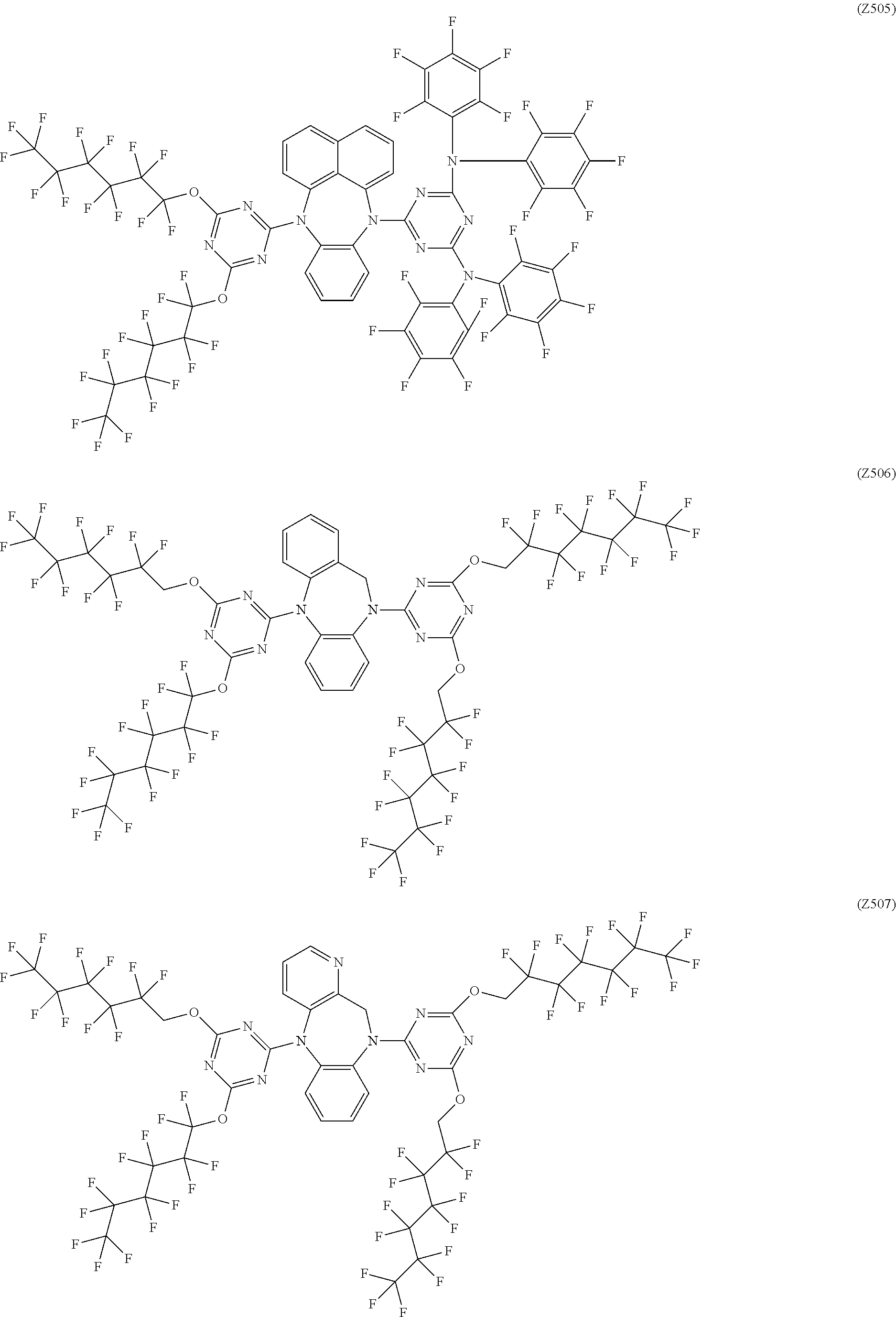

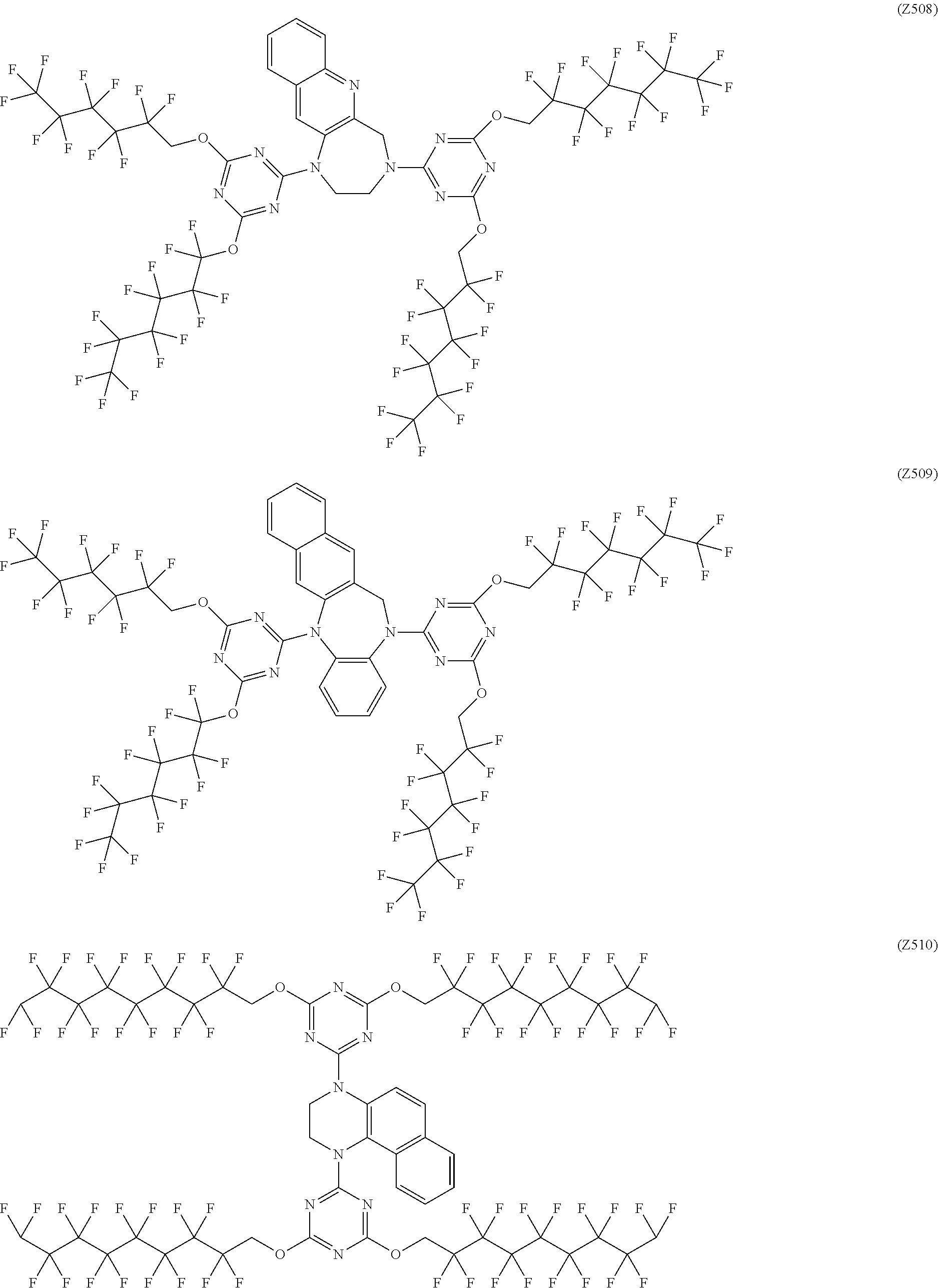

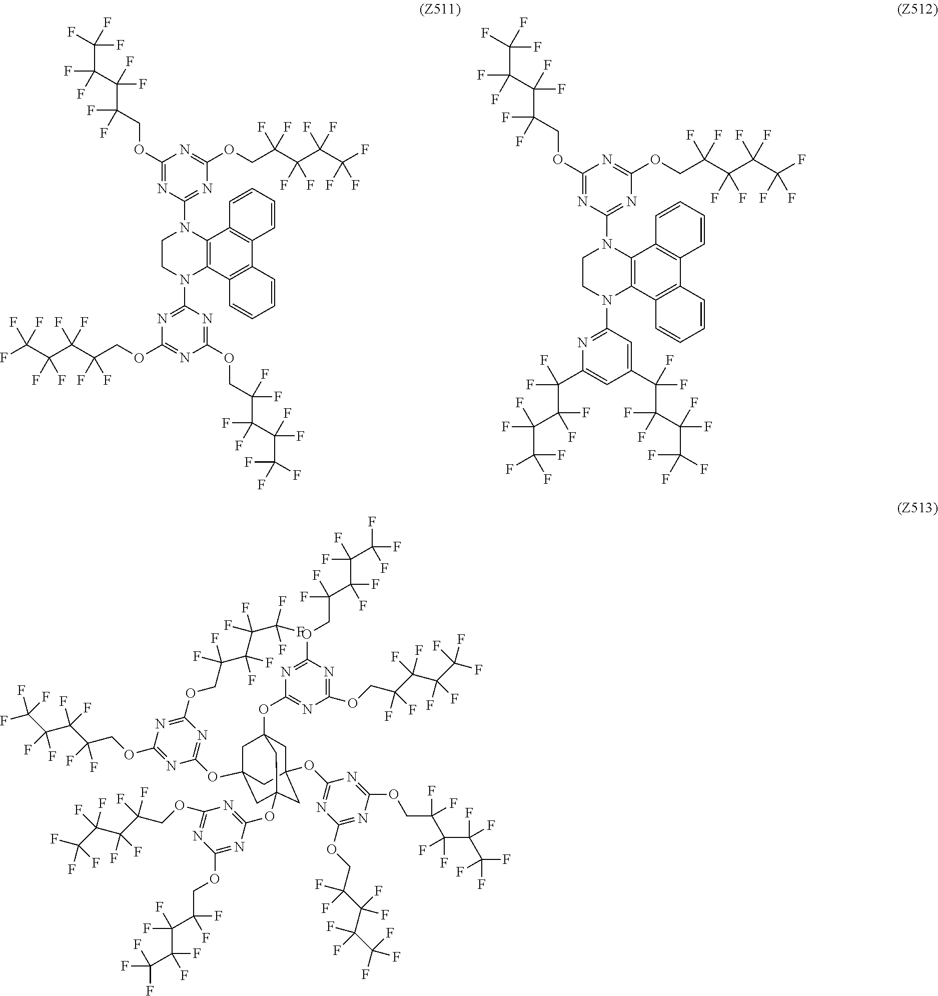

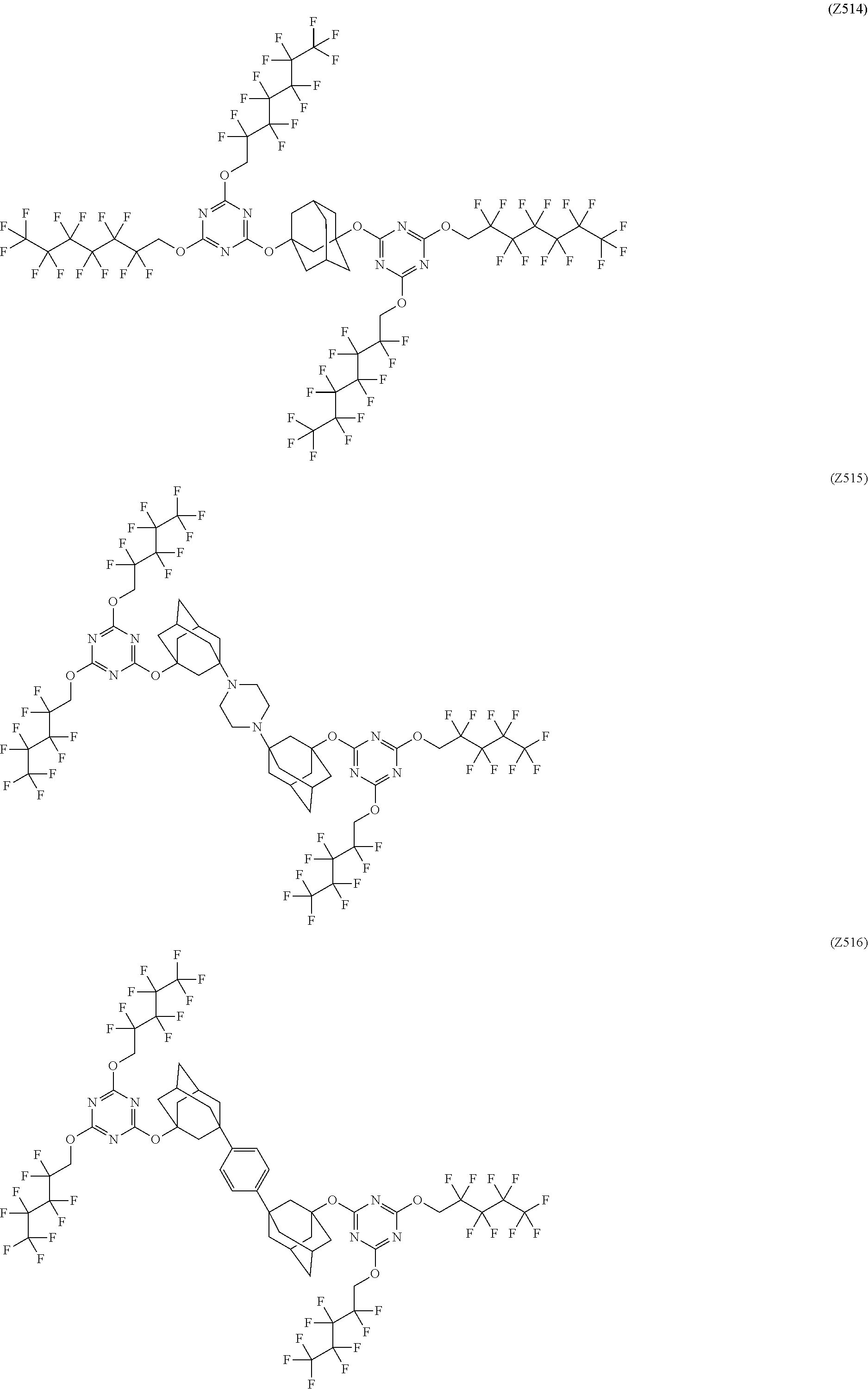

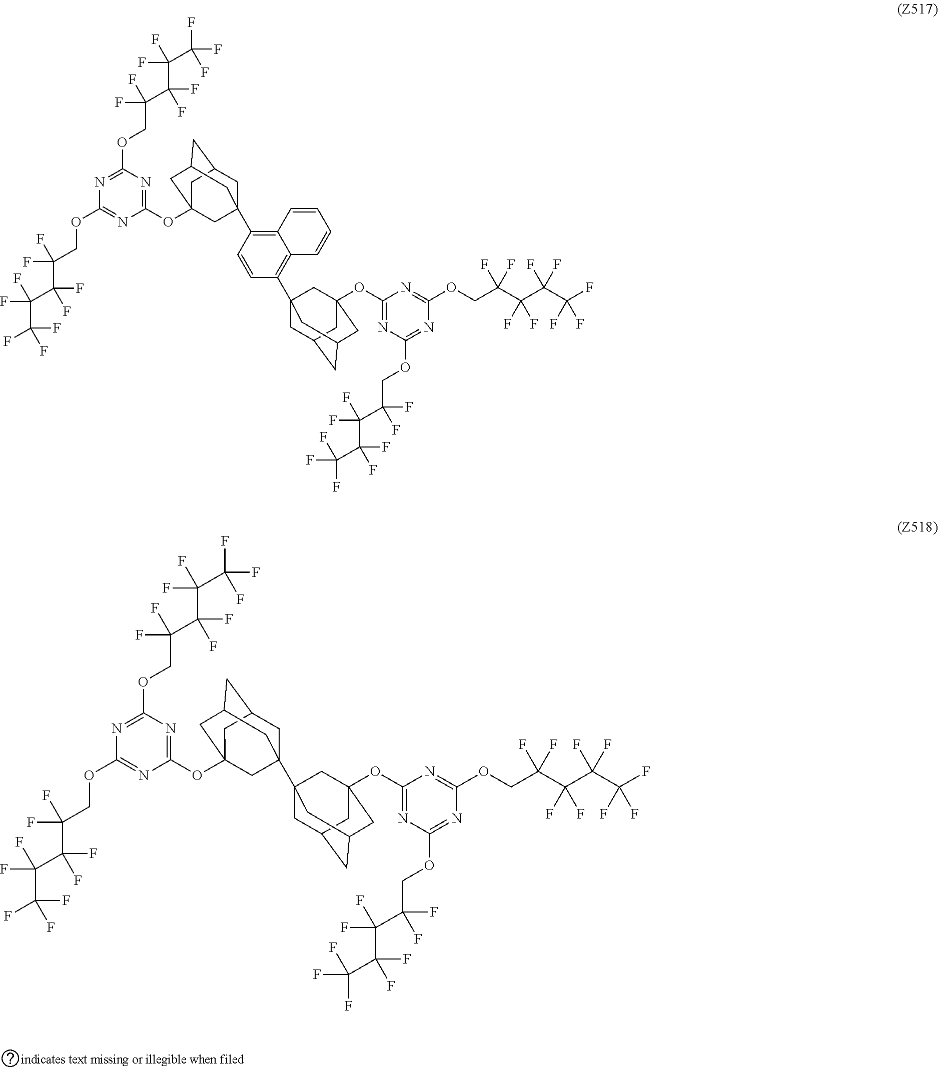 [SMILES: FC(F)(F)C(F)(F)C(F)(F)C(F)(F)COc1nc(OCC(F)(F)C(F)(F)C(F)(F)C(F)(F)F)nc(OC23CC4CC(C2)CC(c2ccc(C56CC7CC(CC(Oc8nc(OCC(F)(F)C(F)(F)C(F)(F)C(F)(F)F)nc(OCC(F)(F)C(F)(F)C(F)(F)C(F)(F)F)n8)(C7)C5)C6)c5ccccc25)(C4)C3)n1 ; FC(F)(F)C(F)(F)C(F)(F)C(F)(F)COc1nc(OCC(F)(F)C(F)(F)C(F)(F)C(F)(F)F)nc(OC23CC4CC(C2)CC(C25CC6CC(CC(Oc7nc(OCC(F)(F)C(F)(F)C(F)(F)C(F)(F)F)nc(OCC(F)(F)C(F)(F)C(F)(F)C(F)(F)F)n7)(C6)C2)C5)(C4)C3)n1]

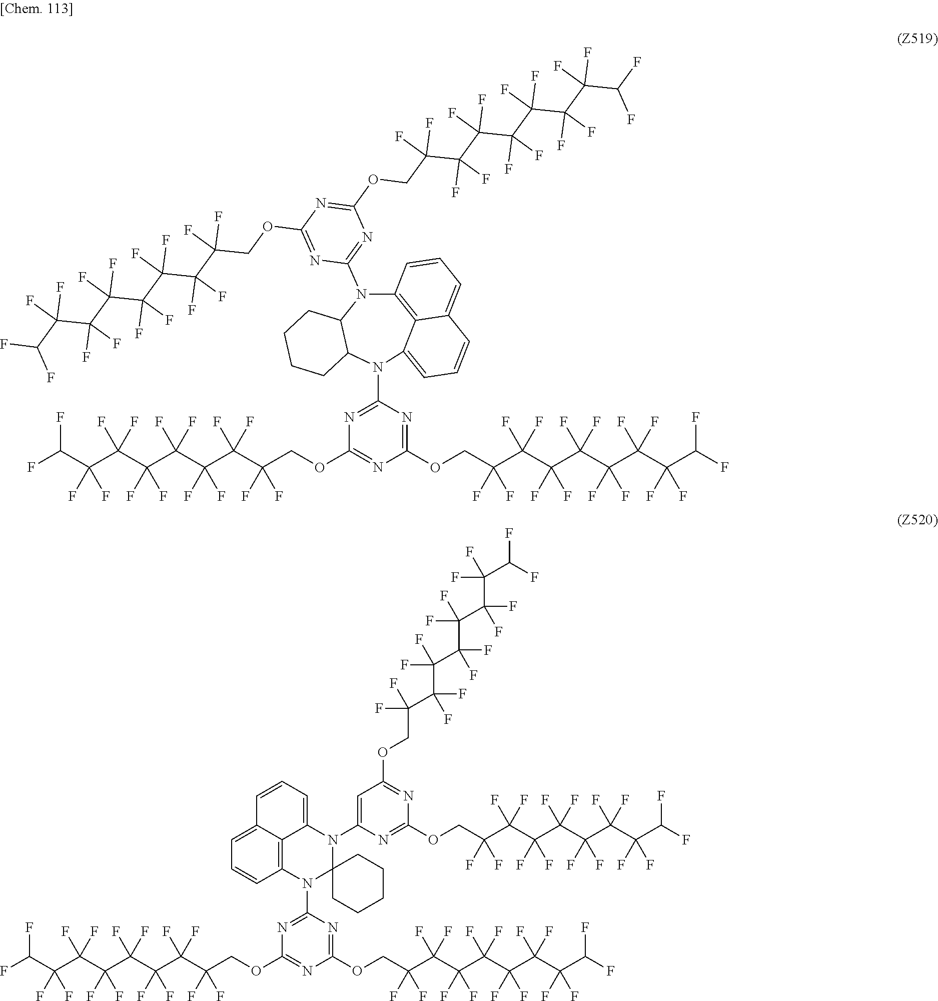

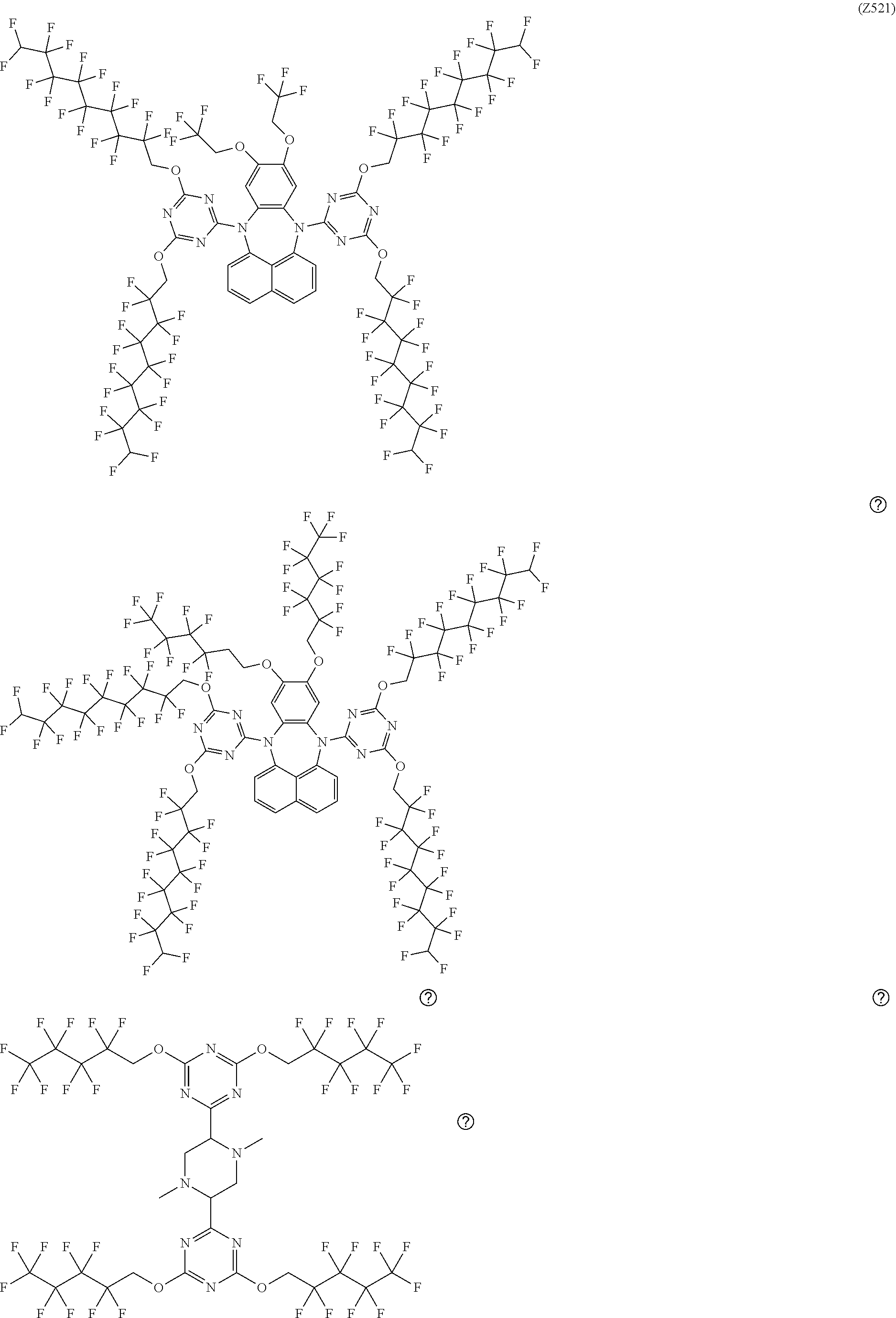

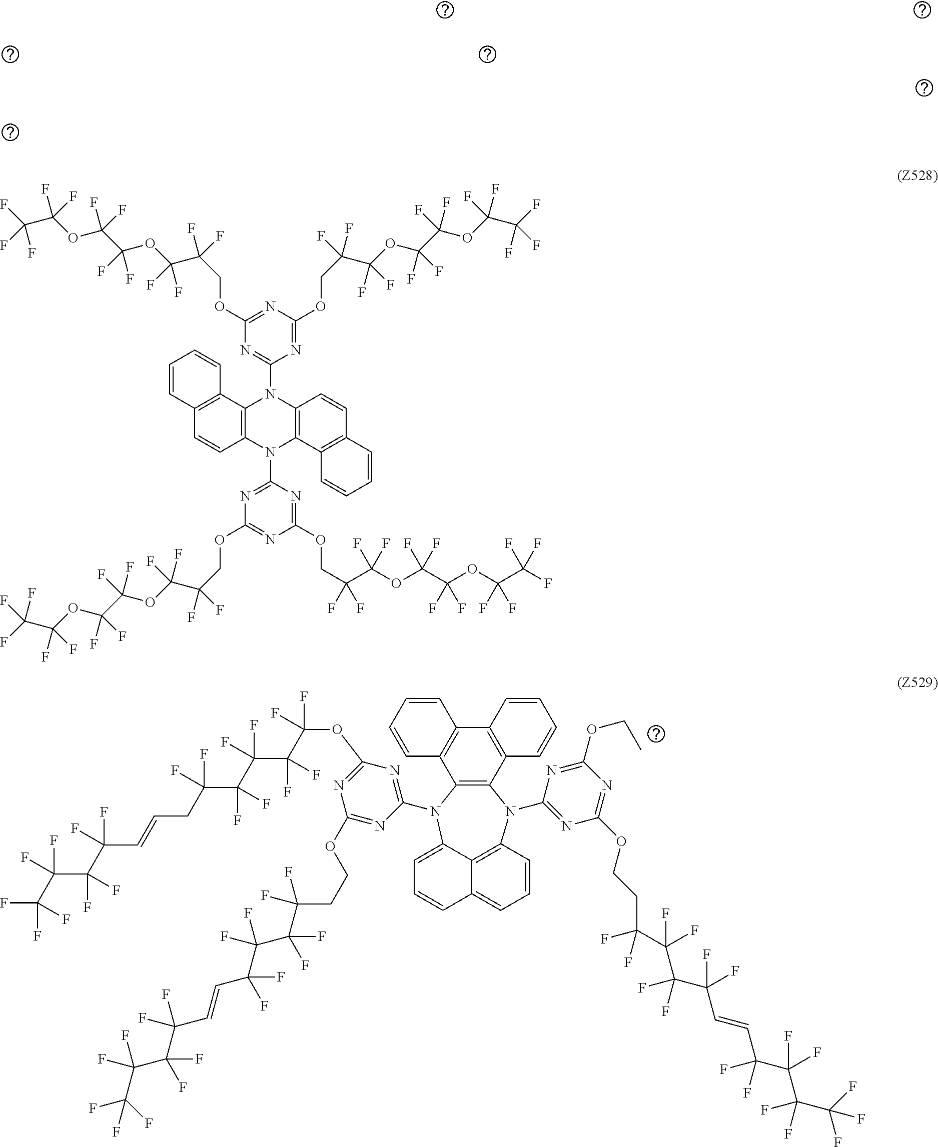

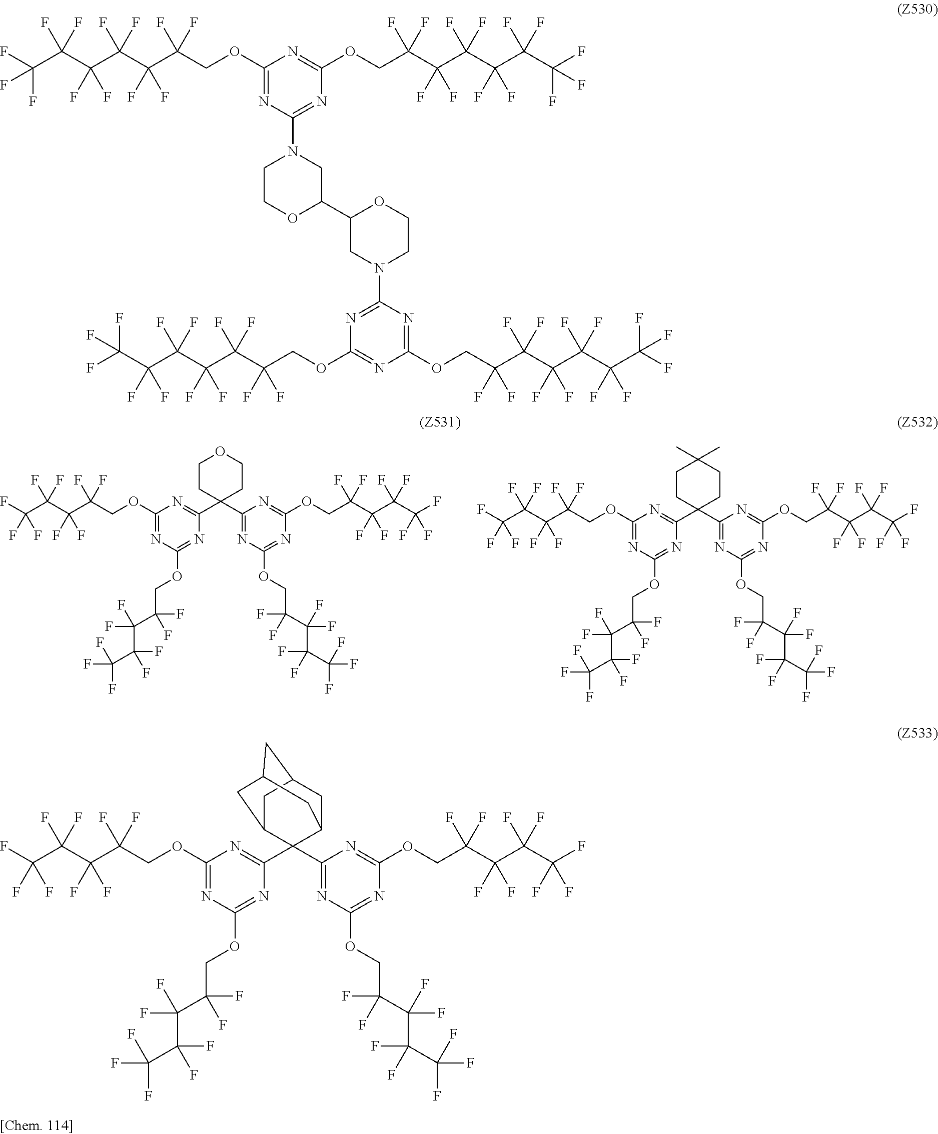

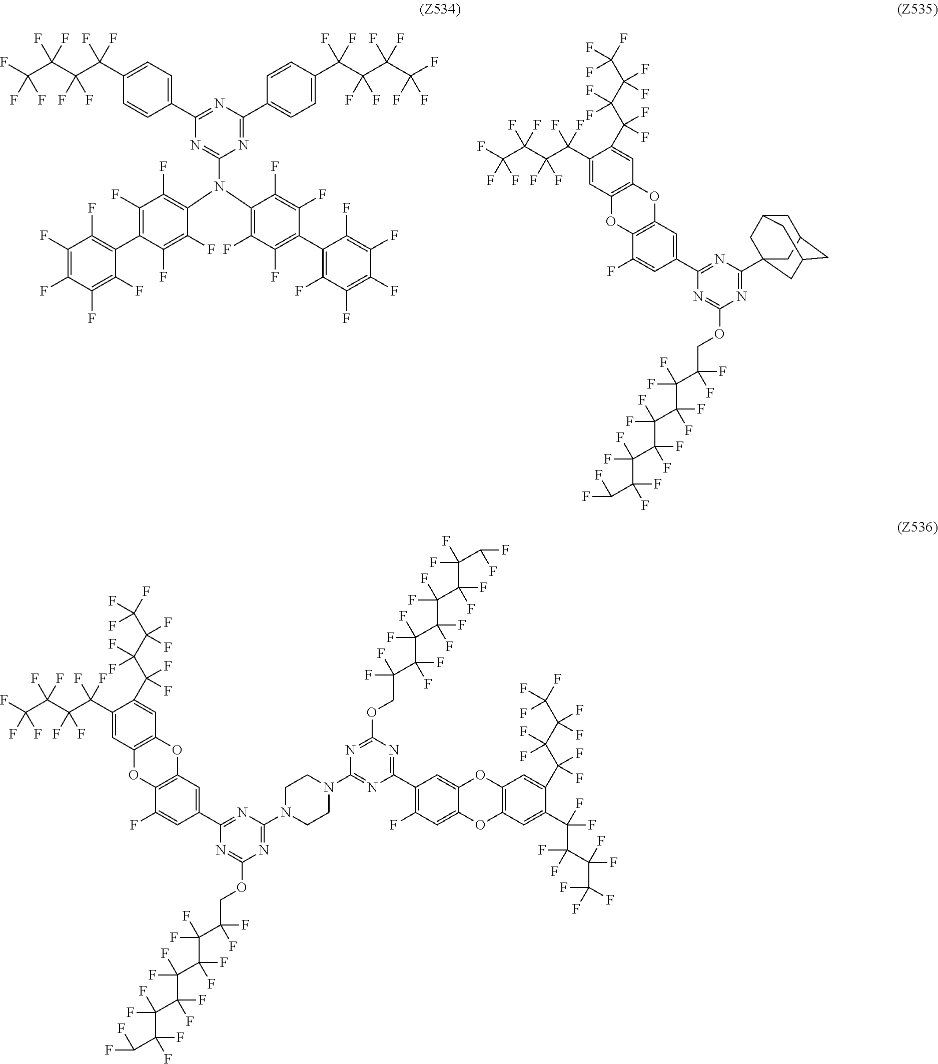

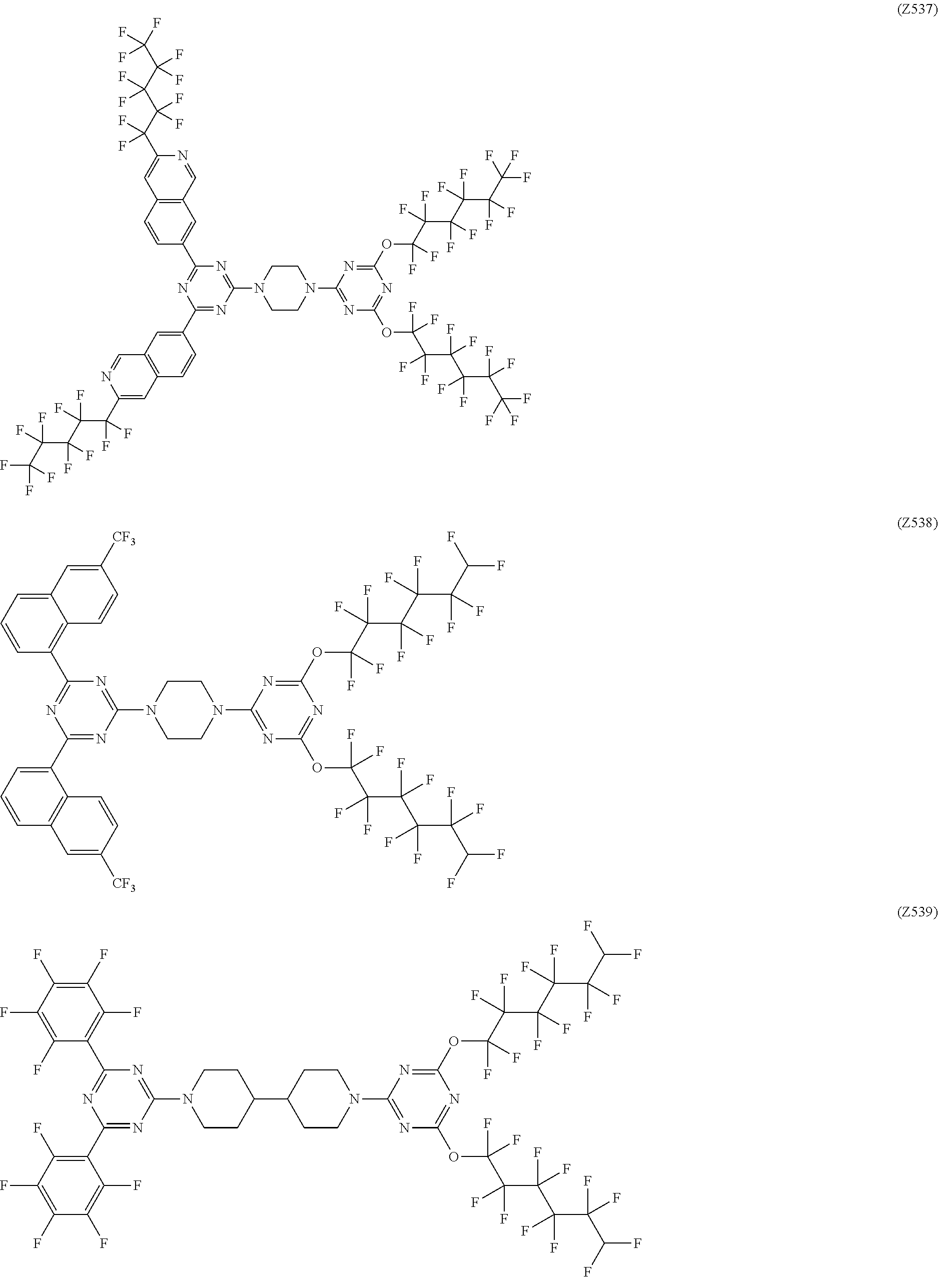

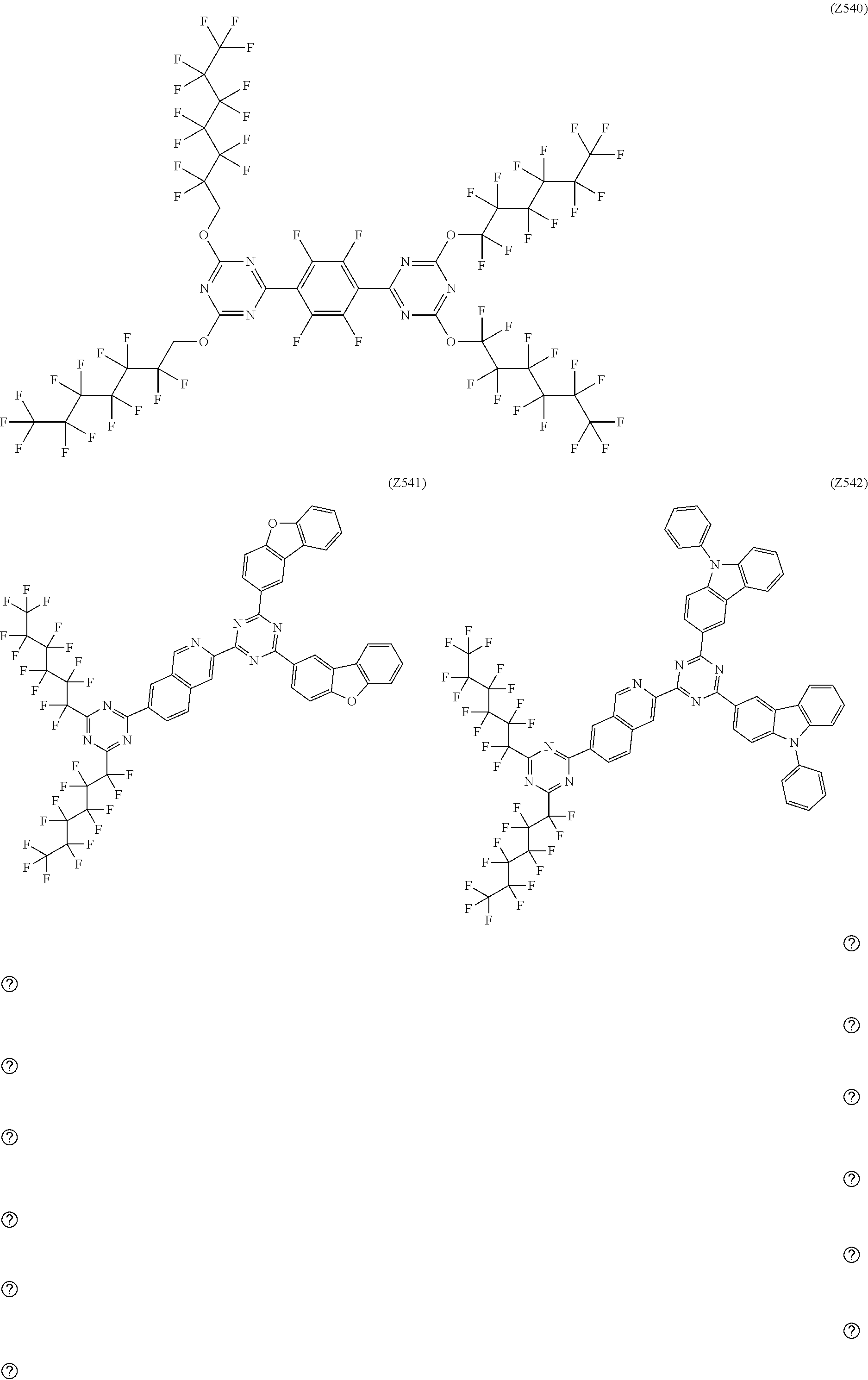

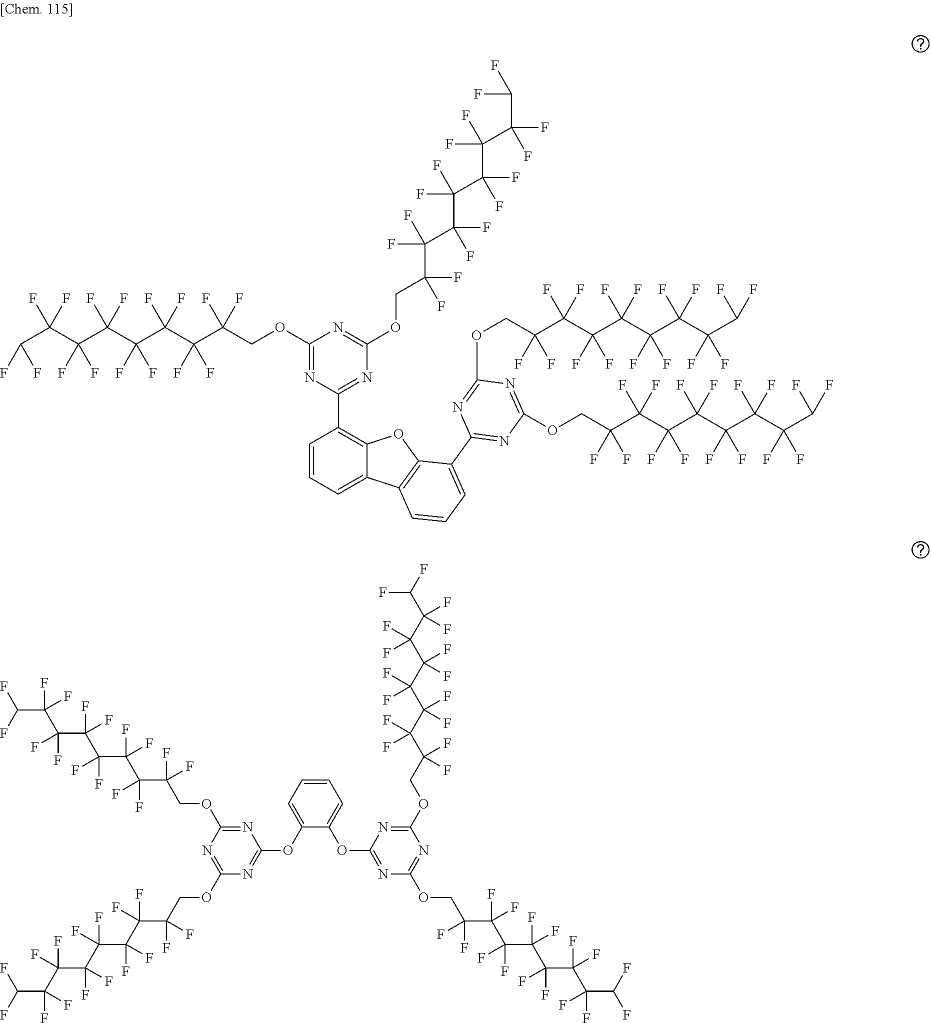

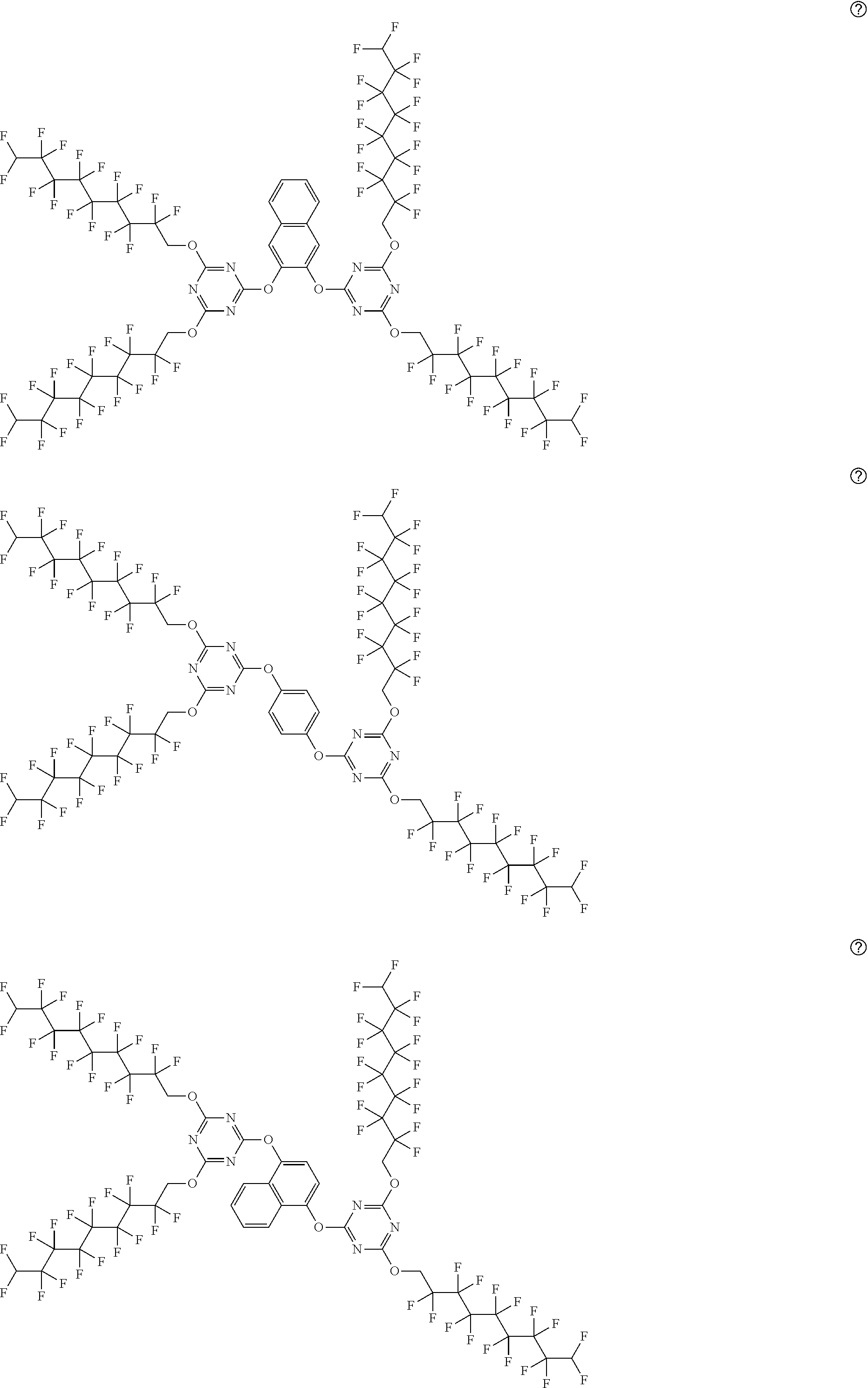

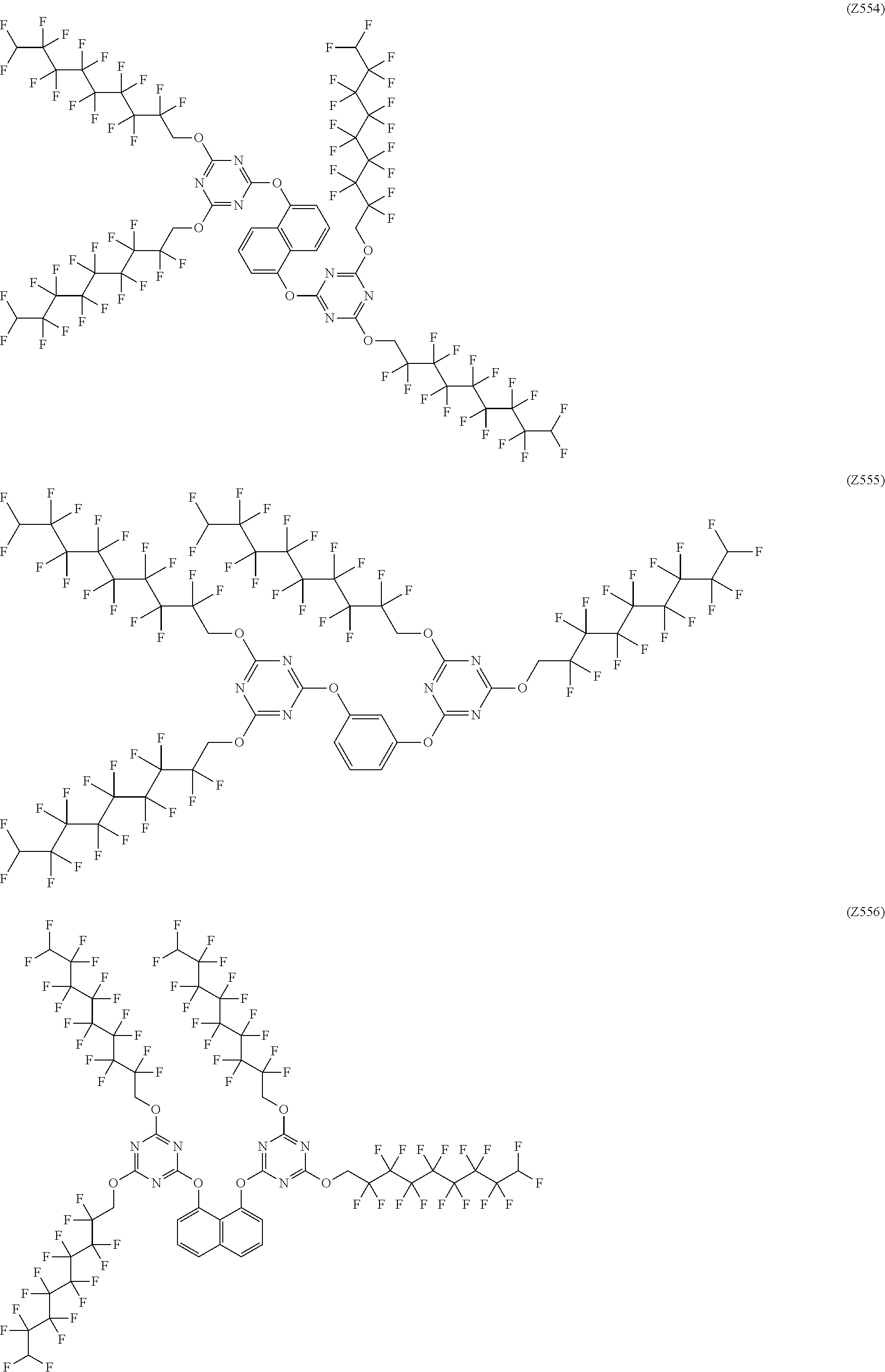

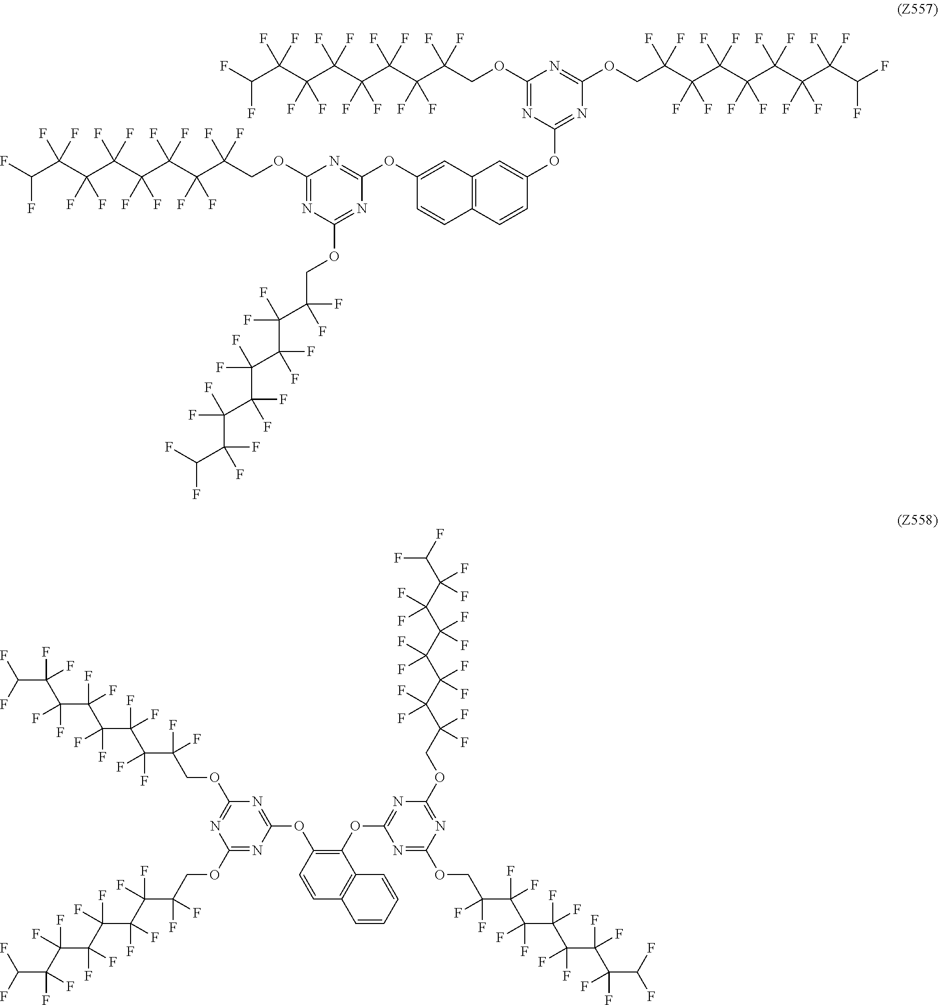

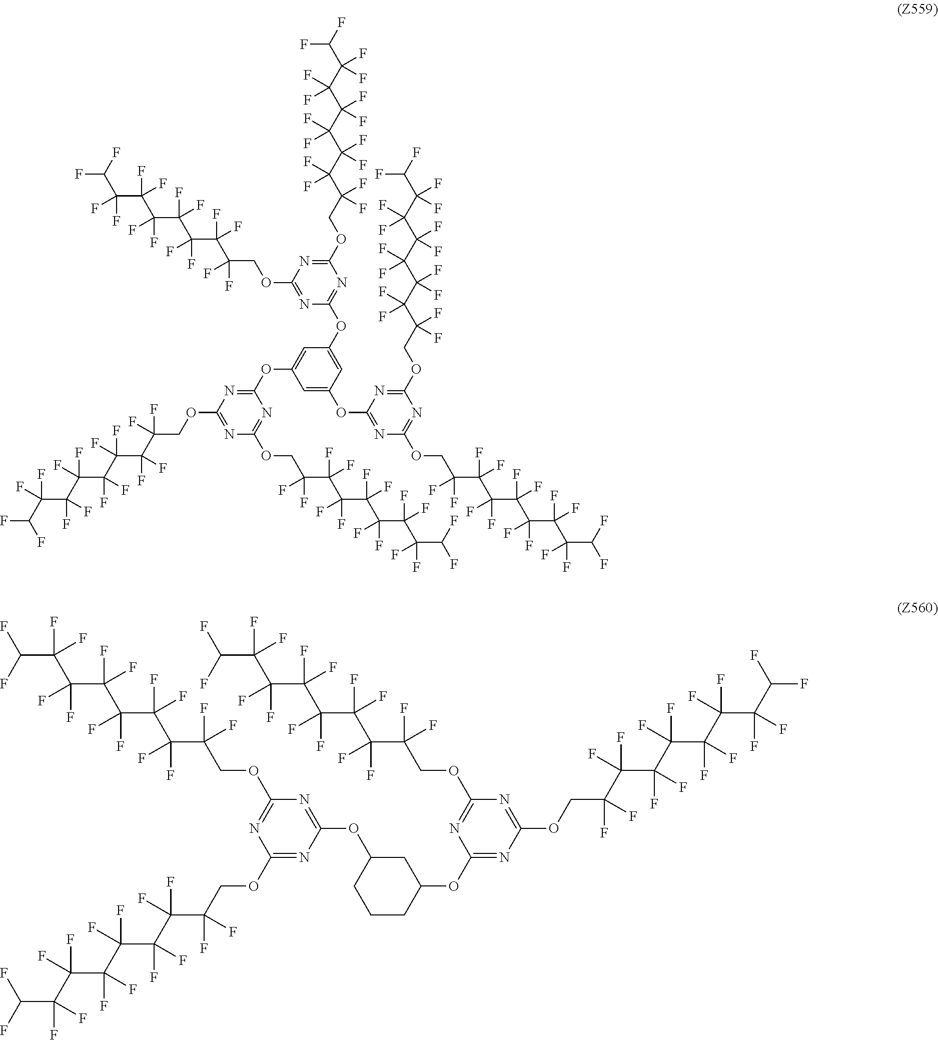

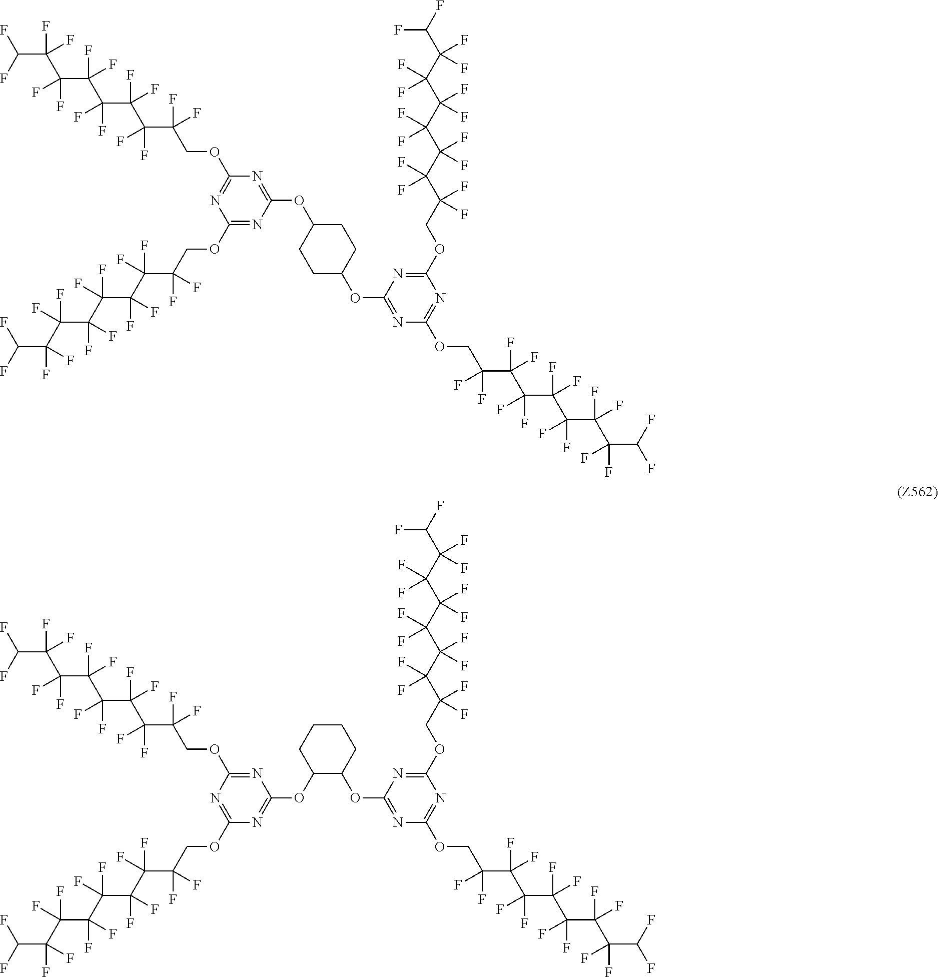

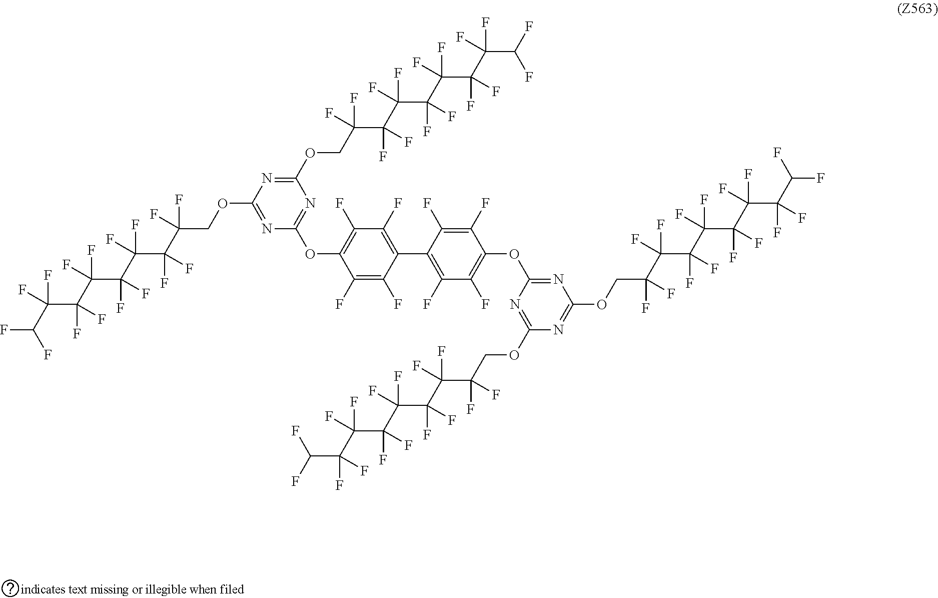 [SMILES: Fc1c(F)c(-c2c(F)c(F)c(Oc3nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n3)c(F)c2F)c(F)c(F)c1Oc1nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n1]

### [Thin Film for Metal Patterning]

A thin film for metal patterning according to an embodiment of the present disclosure contains a material for metal patterning and is capable of patterning a metal film or a metal multilayer film,
wherein the material for metal patterning
contains a compound represented by the formula (A1), (B1), (101), (102), (103), (104) or (105), and
the metal film or the metal multilayer film contains at least one selected from the group consisting of ytterbium, magnesium, silver, aluminum and an alloy of magnesium and silver.

The material for metal patterning contained in the thin film for metal patterning is the same as the material for metal patterning containing the compound described above.

The water contact angle of the thin film for metal patterning is preferred in the following order: 90 degrees or more, 91 degrees or more, 92 degrees or more, 93 degrees or more, 94 degrees or more, 95 degrees or more, 96 degrees or more, 97 degrees or more, 98 degrees or more, 99 degrees or more and 100 degrees or more.

### [Organic Electroluminescent Element]

An organic electroluminescent element according to an embodiment of the present disclosure includes a negative electrode,
wherein the negative electrode
contains at least one selected from the group consisting of ytterbium, magnesium, silver, aluminum and an alloy of magnesium and silver, and
is patterned with a material for metal patterning, and
the material for metal patterning contains
a compound represented by the formula (A1), (B1), (101), (102), (103), (104) or (105).

In the organic electroluminescent element, the material for metal patterning used for patterning is the same as the material for metal patterning containing the compound described above.

### [Method for Forming Metal Pattern]

A method for forming a metal pattern according to an embodiment of the present disclosure includes
forming an organic material pattern containing the material for metal patterning, and
applying a metallic material to a region where the organic material pattern is formed and a region where the organic material pattern is not formed to form a metal pattern in the region where the organic material pattern is not formed.

The material for metal patterning is used by forming a film on a portion where adhesion of metal is desired to be suppressed. The portion where the adhesion of the metallic material is desired to be suppressed corresponds to a region for forming the organic material pattern. A portion other than the portion where the adhesion of the metallic material is desired to be suppressed corresponds to the region where the organic material pattern is not formed. The region where the organic material pattern is not formed is a region where adhesion of the metallic material is promoted and a metal pattern is to be formed.

A method for forming an organic material pattern (a film-forming method) may be, but is not limited to, a known method, such as a vacuum deposition method, a spin coating method, a casting method, a dip coating method, a die coating method, a bar code method, an offset method, a spray coating method, an ink jet method, a screen method, an offset method, a flexographic method, a gravure method or a microcontact method. After a film is formed, the film may be annealed in a temperature environment higher than room temperature. The organic material pattern may have any film thickness.

Another organic molecular material, a polymer or the like may be added to the material for metal patterning, provided that the formation of a metal film on the film surface can be suppressed.

A base on which the organic material pattern is formed may be a metal or a non-metal and is, for example, but not limited to, an organic film, a metal film, an oxide film or an inorganic film. A material of a substrate may be, but is not limited to, glass, plastic, metal, ceramic or any other material.

When a base for forming the organic material pattern is an organic film, it may be, for example, a tris(8-quinolinolato)aluminum derivative, an imidazole derivative, a benzimidazole derivative, a triazine derivative, a pyrimidine derivative, a pyridine derivative, a pyrazine derivative, a quinoline derivative, a quinoxaline derivative, an oxadiazole derivative, a phosphole derivative, a silole derivative, a phosphine oxide derivative or the like.

When a material for metal patterning is used to form a metal pattern, the type of metallic material is preferably, but not limited to, an alkali metal, an alkaline-earth metal, a transition metal or a periodic table group 13 metal, such as lithium, sodium, potassium, rubidium, cesium, beryllium, magnesium, calcium, strontium, barium, aluminum, scandium, vanadium, zinc, yttrium, indium, cerium, samarium, europium, terbium, ytterbium, gold, silver, platinum, copper, iron, palladium, molybdenum, manganese, titanium, cobalt, nickel, tungsten, tin, chromium or an alloy containing one or more of these metals. The alloy is, for example, a magnesium-silver alloy, magnesium-indium alloy, a magnesium-aluminum alloy, an indium-silver alloy, a lithium-aluminum alloy, a lithium-magnesium alloy, a lithium-indium alloy, a calcium-aluminum alloy or the like.

A method for forming a metal pattern may be, but is not limited to, a dry process, such as a vacuum deposition method or a sputtering method, an ink jet method using a metal nano-ink or the like. The metal pattern may have any film thickness.

Applying a metallic material to a region where an organic material pattern is formed and a region where the organic material pattern is not formed can instantaneously form a film containing the metallic material in both regions. However, the metal is less likely to adhere to the region where the organic material pattern is formed, and a metal pattern is naturally formed only in the region where the organic material pattern is not formed.

A more specific method for forming a metal pattern is, for example, the following method 1) or 2).
1) The material for metal patterning is vapor-deposited in a desired pattern using a metal mask or the like.
2) A metal is then vapor-deposited to form an electrode only on a portion where the material for metal patterning is not formed. Thus, a metal electrode of a negative pattern with respect to the deposition pattern of the material for metal patterning is formed.

The area and linewidth of the patterned metal electrode can be arbitrarily adjusted by the patterning shape of the material for metal patterning.

A protective film may be provided on the patterning film formed by patterning the metal. The protective film is, for example, but not limited to, an organic film, an oxide film or an inorganic film.

When the protective film is an organic film, for example, a triazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a silazane derivative, an aniline copolymer, an electrically conductive polymer oligomer (particularly a thiophene oligomer), a porphyrin compound, an aromatic tertiary amine compound, a carbazole compound, a styrylamine compound, a triazine derivative, a pyrimidine derivative or the like can be used.

When the protective film is an inorganic film, for example, silicon nitride, silicon oxide or the like can be used.

Using a material for metal patterning and a method for forming a metal pattern according to an embodiment of the present disclosure, it is possible to pattern a metal electrode of a solar cell, a photosensor, an image sensor, an organic electroluminescent (EL) element, an organic solar cell, an organic sensor, an organic transistor or the like or to form a metal wire on a circuit board.

A material for metal patterning according to an embodiment of the present disclosure can also be applied to a vapor deposition process.

Furthermore, a material for metal patterning according to an embodiment of the present disclosure has 7 or more fluorine atoms in the molecule to suppress the formation of a thin metal film on a film surface.

### [Electronic Device]

An electronic device according to an embodiment of the present disclosure includes the material for metal patterning. As described above, an organic material pattern containing the material for metal patterning is formed when a metal pattern is formed, and the organic material pattern is formed together with the metal pattern. Thus, an electronic device according to an embodiment of the present disclosure includes an organic material pattern containing the material for metal patterning together with a metal pattern.

The electronic device is, for example, a solar cell, a photosensor, an image sensor, an organic EL element, an organic solar cell, an organic sensor, an organic transistor or the like. These electronic devices include patterning of a metal electrode, a metal wire on a circuit board or the like. In other words, the electronic device according to the present embodiment can be produced using the method for forming a metal pattern, provided that the electronic device includes patterning of a metal electrode or a metal wire on a circuit board. Such an electronic device has a high precision metal pattern.

### EXAMPLES

The present invention is described in more detail below on the basis of examples. However, the present invention is not to be interpreted as being limited by these examples.

NMR measurement was performed with the following apparatus.

### [NMR Measurement]

Measuring apparatus: JNM-ECZ400S manufactured by JEOL
Transmittance was measured with the following apparatus.

### [Transmittance Measurement]

Measuring apparatus: V-750 manufactured by JASCO Corporation
Measurement range: 550 to 800 nm

### Example 1 Synthesis of Compound (Z1)

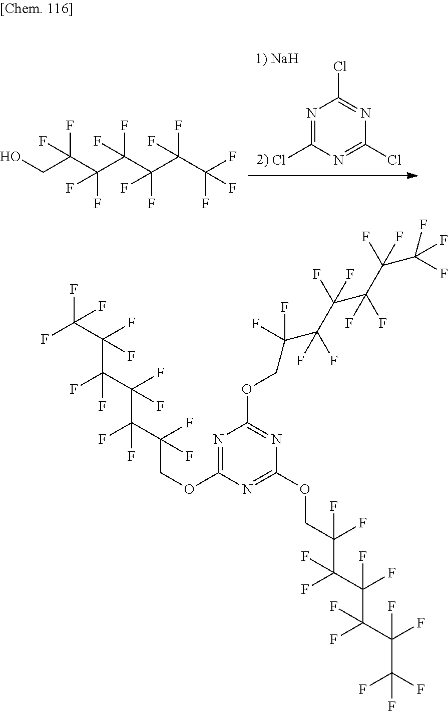

In a nitrogen atmosphere, sodium hydride (3.30 g) was suspended in tetrahydrofuran (120 ml) and was stirred at 0° C., and 1H, 1H-tridecafluoro-1-heptanol (25.0 g) was added dropwise to the suspension over 20 minutes. After completion of the dropwise addition, the suspension was returned to room temperature and was stirred for 2 hours. Furthermore, a tetrahydrofuran solution (40 ml) of cyanuric chloride (4.4 g) was added dropwise to the suspension at 0° C. over 20 minutes. The dropwise addition was followed by stirring at 60° C. for 5 hours. After cooling to room temperature, water (20 ml) was added to the reaction liquid, and the precipitated solid was collected by filtration to produce a white target compound (Z1) (amount: 14.4 g, yield: 53.7%).

1H-NMR (400 MHZ, THF-d8); 5.17 (t, J=14.00, 6H)

19F-NMR (376.4 MHZ, THE-d8) δ (ppm): −80.3 (m, 9F), −118.6 (m, 6F), −121.2 (m, 6F), −121.9 (m, 6F), −122.2 (m, 6F), −125.3 (m, 6F).

### Example 1′ (Evaluation of Adhesiveness of Compound (Z1) to Metal)

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 100-nm film of the compound (Z1) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z1) was vapor-deposited, and a 2 mm×1 mm transparent region was formed.

### Example 2 Synthesis of Compound (Z174)

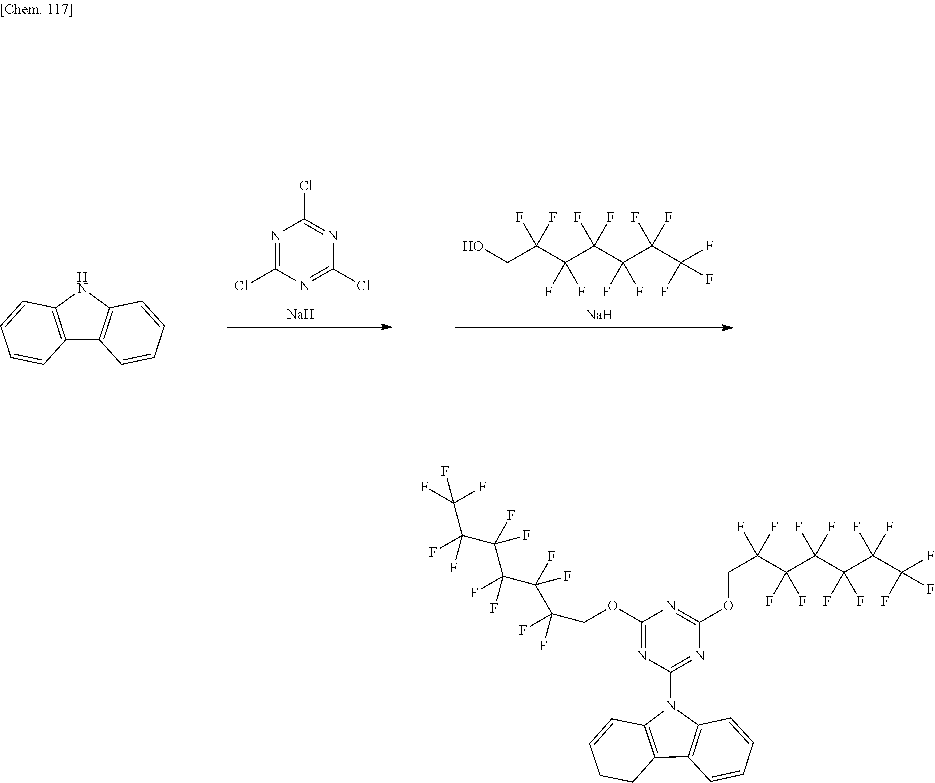

In a nitrogen atmosphere, sodium hydride (0.72 g) was suspended in tetrahydrofuran (40 ml) and was stirred at 0° C., and a tetrahydrofuran solution (30 ml) of 9H-carbazole (2.5 g) was added dropwise to the suspension over 60 minutes. After completion of the dropwise addition, the suspension was returned to room temperature and was stirred for 2 hours. Furthermore, the suspension was added dropwise to a tetrahydrofuran solution (40 ml) of cyanuric chloride (2.8 g) at 0° C. over 20 minutes. After completion of the dropwise addition, the suspension was returned to room temperature and was stirred for 2 hours (suspension 2-1).

In a nitrogen atmosphere, sodium hydride (1.8 g) was suspended in tetrahydrofuran (30 ml) and was stirred at 0° C., and 1H, 1H-tridecafluoro-1-heptanol (13 g) was added dropwise to the suspension over 20 minutes. After completion of the dropwise addition, the suspension was returned to room temperature and was stirred for 2 hours (suspension 2-2).

The suspension 2-2 was added dropwise to the suspension 2-1 at 0° C. over 20 minutes. After completion of the dropwise addition, the suspension was returned to room temperature and was stirred for 2 hours. Water (20 ml) was added to the reaction liquid, the liquid was separated using pure water and chloroform, and the organic layer was washed with saturated aqueous sodium chloride. The organic layer was dried over anhydrous magnesium sulfate and was then purified by silica gel column chromatography to produce a white target compound (Z174) (amount: 3.5 g, yield: 25%).

The compound was identified by FDMS measurement.
FDMS: 942

### Example 2′ (Evaluation of Adhesiveness of Compound (Z174) to Metal)

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z174) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z174) was vapor-deposited, and a 2 mm×1 mm transparent region was formed.

### Example 3 Synthesis of Compound (Z177)

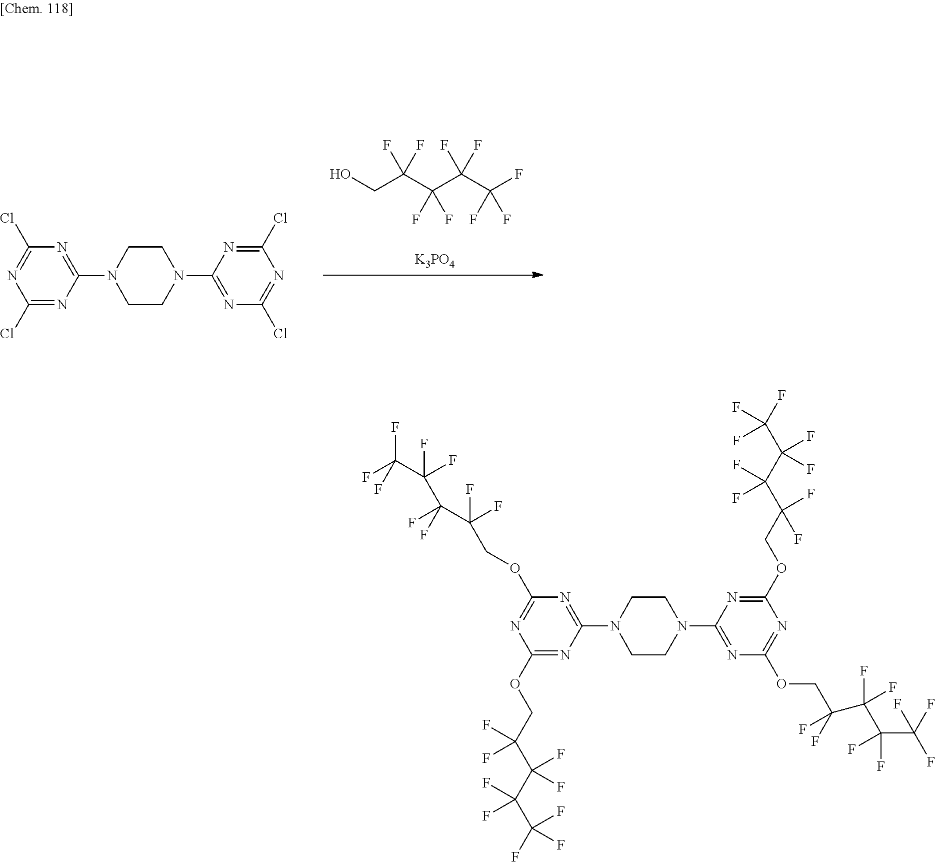

In a nitrogen atmosphere, 1,4-bis(4,6-dichloro-1,3,5-triazin-2-yl) piperidine (1.00 g), tripotassium phosphate (8.00 g) and 1H, 1H-nonafluoro-1-pentanol (2.90 g) were suspended in tetrahydrofuran (30 ml) and were stirred at 70° C. for 24 hours. After cooling to room temperature, water (100 ml) was added to the reaction liquid, and the precipitated solid was collected by filtration to produce a white target compound (Z177) (amount: 2.87 g, yield: 88.7%).

The compound was identified by FDMS measurement.
FDMS: 1236

### Example 3′ (Evaluation of Adhesiveness of Compound (Z177) to Metal)

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z177) was formed at a vapor-deposition rate of 0.2 nm/s on a glass substrate on which a metal mask with a 2 mm×1 mm opening was disposed. The metal mask was then removed, 1 nm of ytterbium was vapor-deposited at a vapor-deposition rate of 0.01 nm/s, and 15 nm of silver and magnesium (1/9) was then vapor-deposited at a vapor-deposition rate of 0.1 nm/s. A film of silver and magnesium was not formed in a portion where the compound (Z177) was vapor-deposited, and a 2 mm×1 mm transparent region was formed.

### Example 4 Synthesis of Compound (Z180)

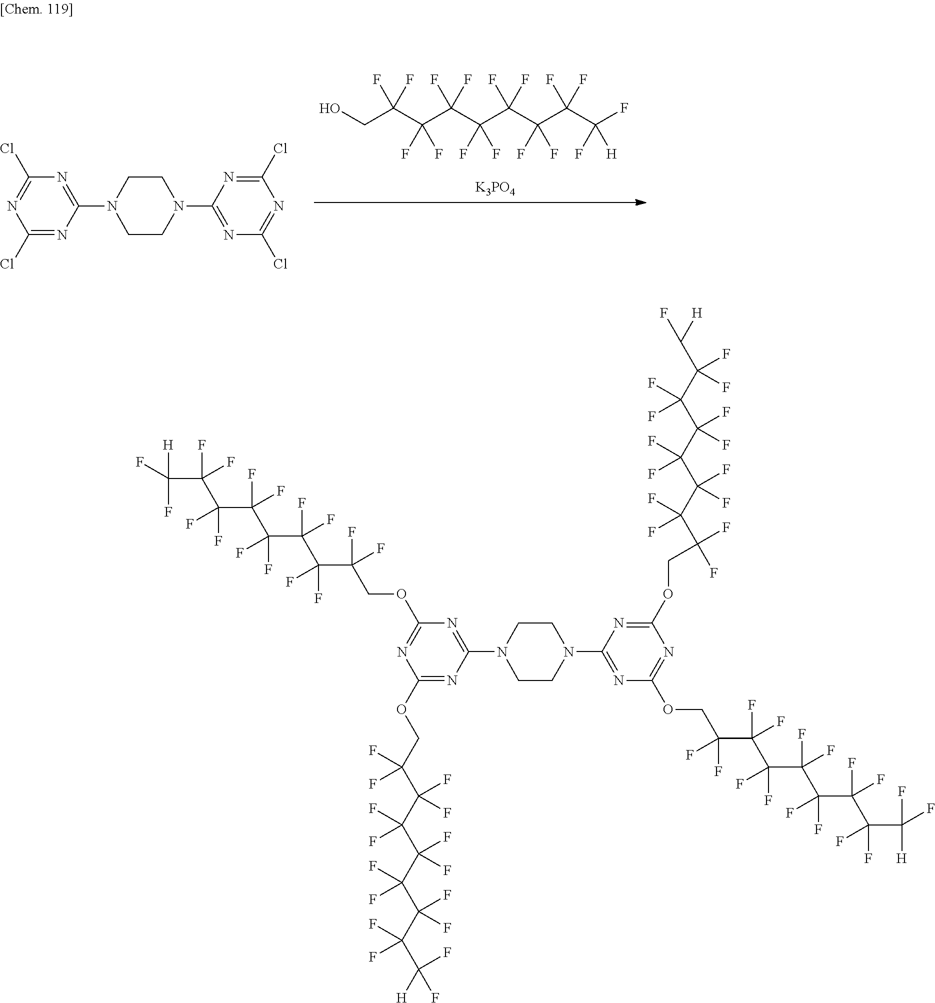

In a nitrogen atmosphere, 1,4-bis(4,6-dichloro-1,3,5-triazin-2-yl) piperidine (2.00 g), tripotassium phosphate (8.00 g) and 1H, 1H, 9H-hexadecafluoro-1-nonal (4.40 g) were suspended in tetrahydrofuran (60 ml) and were stirred at 70° C. for 48 hours. After cooling to room temperature, water (100 ml) was added to the reaction liquid, and the precipitated solid was collected by filtration to produce a white target compound (Z180) (amount: 8.15 g, yield: 79.1%).

The compound was identified by FDMS measurement.
FDMS: 1964

### Example 4′ (Evaluation of Adhesiveness of Compound (Z180) to Metal)

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z180) was formed at a vapor-deposition rate of 0.2 nm/s on a glass substrate on which a metal mask with a 2 mm×1 mm opening was disposed. The metal mask was then removed, 1 nm of ytterbium and lithium fluoride (1/1) was vapor-deposited at a vapor-deposition rate of 0.01 nm/s, and 12 nm of silver and magnesium (1/9) was then vapor-deposited at a vapor-deposition rate of 0.1 nm/s. A film of silver, magnesium, ytterbium and lithium fluoride was not formed in a portion where the compound (Z180) was vapor-deposited, and a 2 mm×1 mm transparent region was formed.

### Example 5 Synthesis of Compound (Z185)

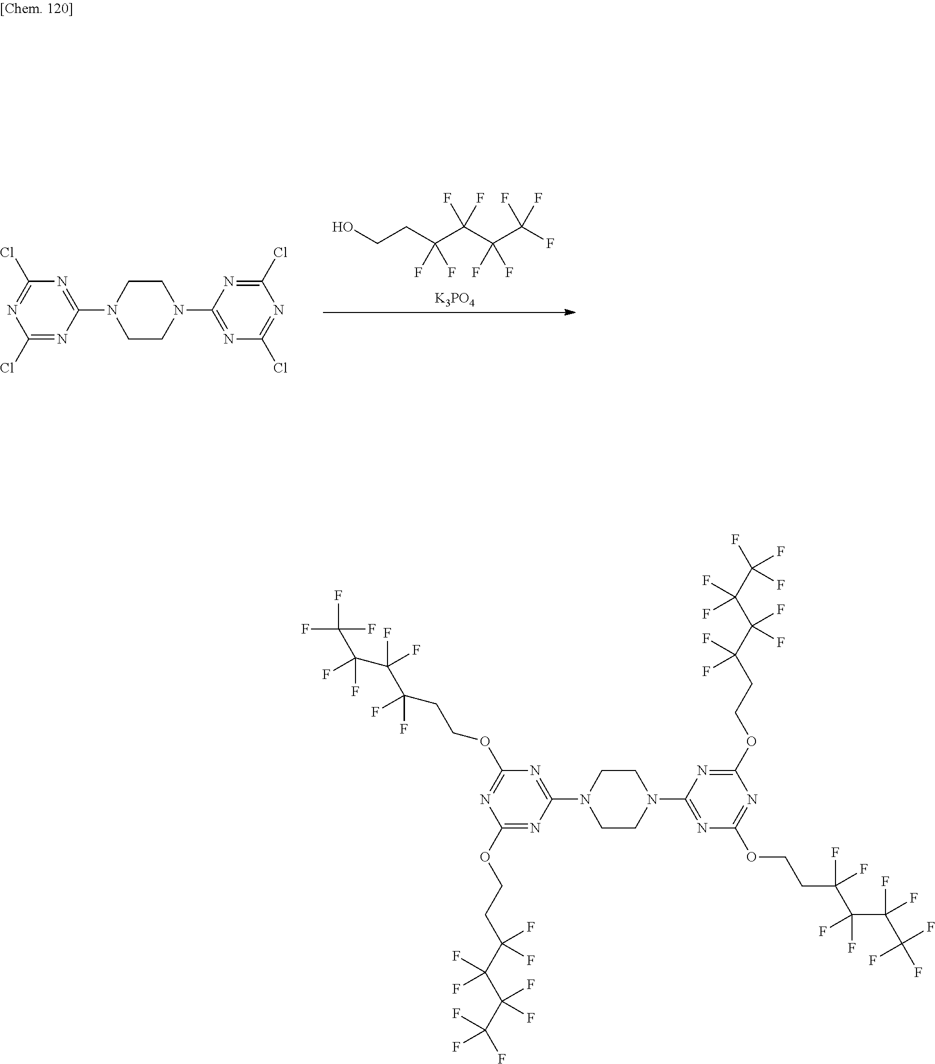

In a nitrogen atmosphere, 1,4-bis(4,6-dichloro-1,3,5-triazin-2-yl) piperidine (1.00 g), tripotassium phosphate (4.60 g) and 1H, 1H-nonafluoro-1-hexanol (3.30 g) were suspended in tetrahydrofuran (60 ml) and were stirred at 70° C. for 92 hours. After cooling to room temperature, water (100 ml) was added to the reaction liquid, and the precipitated solid was collected by filtration to produce a white target compound (Z185) (amount: 2.24 g, yield: 66.2%).

The compound was identified by FDMS measurement.
FDMS: 1292

### Example 5′ (Evaluation of Adhesiveness of Compound (Z185) to Metal)

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z185) was formed at a vapor-deposition rate of 0.2 nm/s on a glass substrate on which a metal mask with a 2 mm×1 mm opening was disposed. The metal mask was then removed, 1 nm of lithium fluoride was vapor-deposited at a vapor-deposition rate of 0.01 nm/s, and 12 nm of silver and magnesium (1/9) was then vapor-deposited at a vapor-deposition rate of 0.1 nm/s. A film of lithium fluoride, silver and magnesium was not formed in a portion where the compound (Z185) was vapor-deposited, and a 2 mm×1 mm transparent region was formed.

### Example 6 Synthesis of Intermediate (S1)

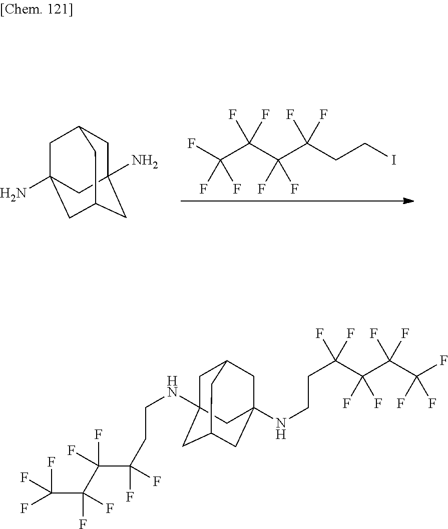 [SMILES: NC12CC3CC(C1)CC(N)(C3)C2 ; FC(F)(F)C(F)(F)C(F)(F)C(F)(F)CCI ; FC(F)(F)C(F)(F)C(F)(F)C(F)(F)CCNC12CC3CC(C1)CC(NCCC(F)(F)C(F)(F)C(F)(F)C(F)(F)F)(C3)C2]

In a nitrogen atmosphere, adamantane diamine (15.0 mmol), 1H, 1H, 2H, 2H-nonafluorohexyl iodide (120.0 mmol) and tripotassium phosphate (60.0 mmol) were suspended in acetonitrile (50 mL) and were stirred at 80° C. for 48 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target intermediate (S1) (yield: 75%).

The compound was identified by FDMS measurement.
FDMS: 658

### Example 7 Synthesis of Compound (Z189)

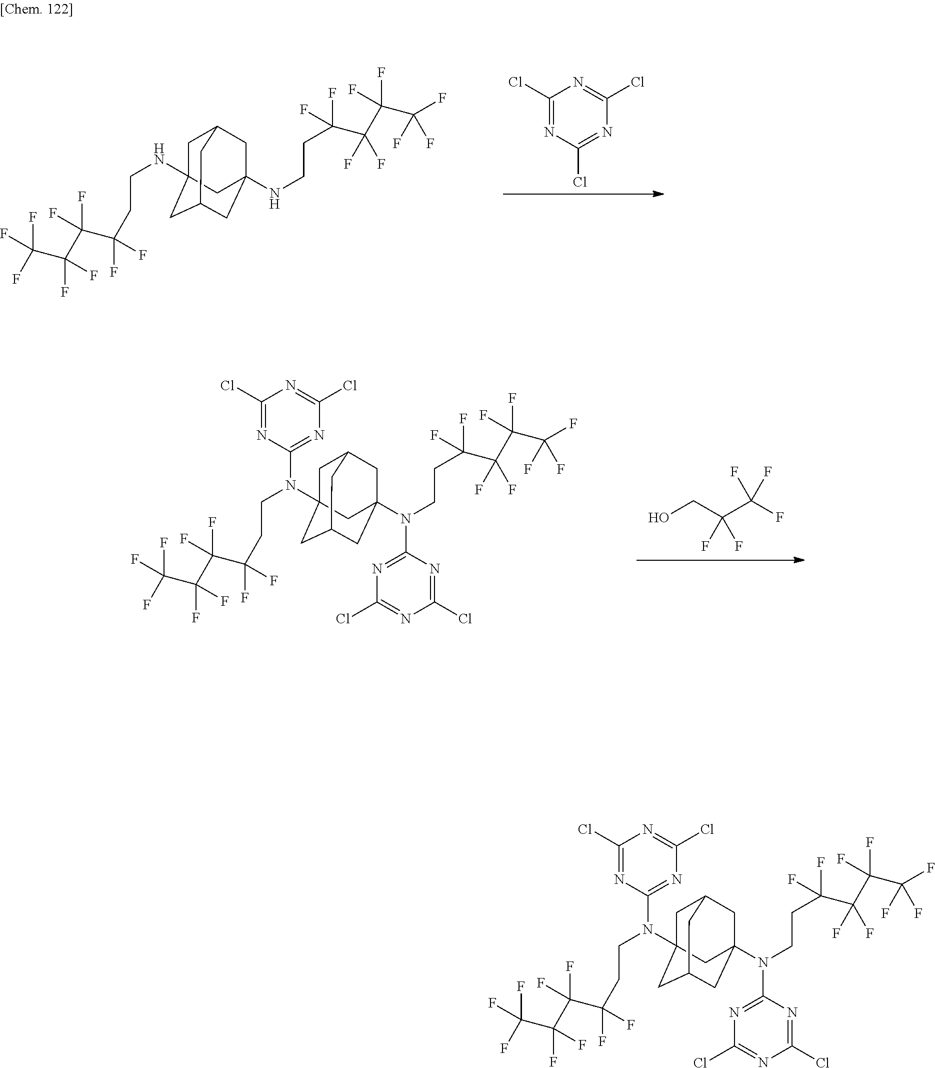 [SMILES: FC(F)(F)C(F)(F)C(F)(F)C(F)(F)CCNC12CC3CC(C1)CC(NCCC(F)(F)C(F)(F)C(F)(F)C(F)(F)F)(C3)C2 ; Clc1nc(Cl)nc(Cl)n1 ; FC(F)(F)C(F)(F)C(F)(F)C(F)(F)CCN(c1nc(Cl)nc(Cl)n1)C12CC3CC(C1)CC(N(CCC(F)(F)C(F)(F)C(F)(F)C(F)(F)F)c1nc(Cl)nc(Cl)n1)(C3)C2 ; OCC(F)(F)C(F)(F)F ; FC(F)(F)C(F)(F)C(F)(F)C(F)(F)CCN(c1nc(Cl)nc(Cl)n1)C12CC3CC(C1)CC(N(CCC(F)(F)C(F)(F)C(F)(F)C(F)(F)F)c1nc(Cl)nc(Cl)n1)(C3)C2]

In a nitrogen atmosphere, sodium hydride (7.0 mmol) was suspended in tetrahydrofuran (30 ml) and was stirred at 0° C., and S1 (3.0 mmol) was added dropwise to the suspension over 20 minutes. After completion of the dropwise addition, the suspension was returned to room temperature and was stirred for 2 hours. Furthermore, a tetrahydrofuran solution (20 ml) of cyanuric chloride (7.0 mmol) was added dropwise to the suspension at 0° C. over 20 minutes. The dropwise addition was followed by stirring at 60° C. for 5 hours. Next, in the reactor cooled to 0° C., in a nitrogen atmosphere, sodium hydride (15.0 mmol) was suspended in tetrahydrofuran (20 ml) and was stirred at 0° C., and pentafluoropropanol (14.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z189) (yield: 34%).

The compound was identified by FDMS measurement.
FDMS: 1408

### Example 7′ (Evaluation of Adhesiveness of Compound (Z189) to Metal)

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z189) was formed at a vapor-deposition rate of 0.2 nm/s on a glass substrate on which a metal mask with a 2 mm×1 mm opening was disposed. The metal mask was then removed, 1 nm of magnesium fluoride was vapor-deposited at a vapor-deposition rate of 0.01 nm/s, and 12 nm of silver and magnesium (1/9) was then vapor-deposited at a vapor-deposition rate of 0.1 nm/s. A film of magnesium fluoride, silver and magnesium was not formed in a portion where the compound (Z189) was vapor-deposited, and a 2 mm×1 mm transparent region was formed.

### Example 8 Synthesis of Compound (Z190)

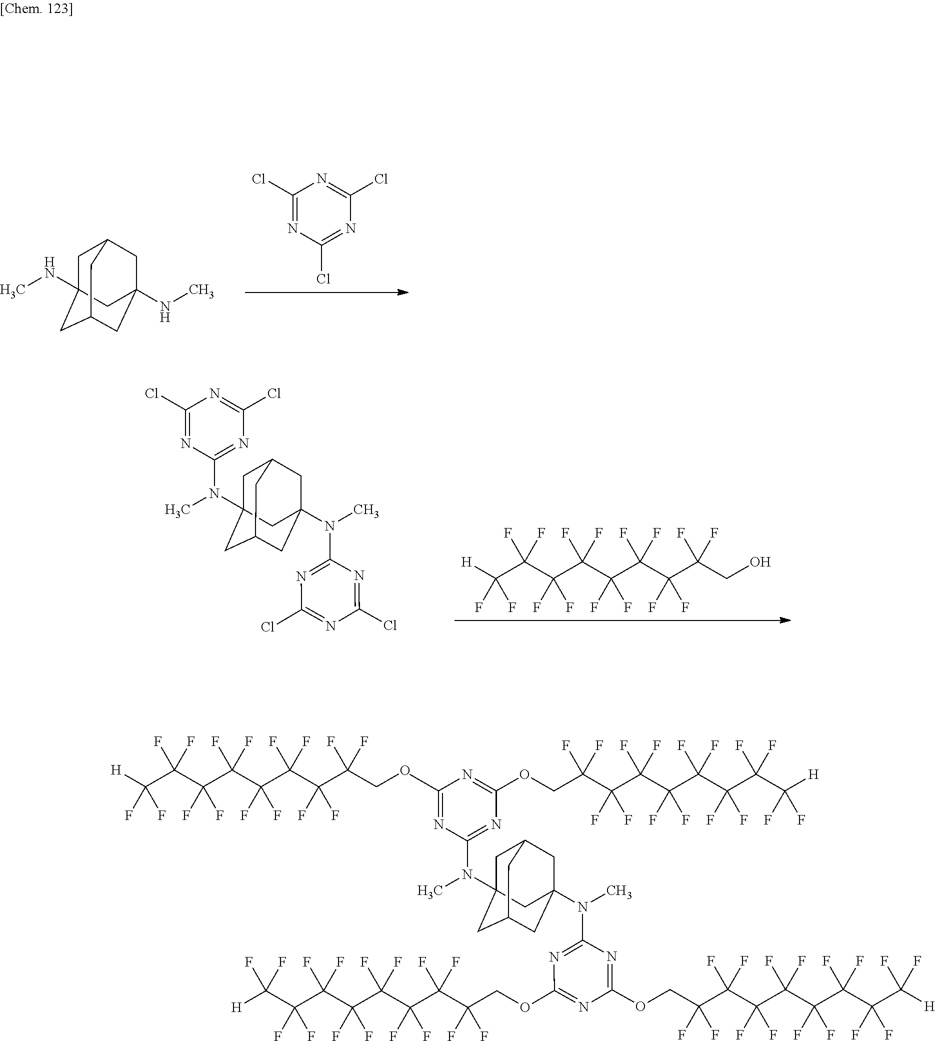

In a nitrogen atmosphere, sodium hydride (7.0 mmol) was suspended in tetrahydrofuran (30 ml) and was stirred at 0° C., and N,N′-dimethyl-1,3-adamantanediamine (3.0 mmol) was added dropwise to the suspension over 20 minutes. After completion of the dropwise addition, the suspension was returned to room temperature and was stirred for 2 hours. Furthermore, a tetrahydrofuran solution (20 ml) of cyanuric chloride (7.0 mmol) was added dropwise to the suspension at 0° C. over 20 minutes. The dropwise addition was followed by stirring at 60° C. for 5 hours. Next, in the reactor cooled to 0° C., in a nitrogen atmosphere, sodium hydride (15.0 mmol) was suspended in tetrahydrofuran (20 ml) and was stirred at 0° C., and 1H, 1H, 9H-hexadecafluoro-1-nonal (14.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z190) (yield: 28%).

The compound was identified by FDMS measurement.
FDMS: 1408

### Example 8′ Evaluation of Adhesiveness of Compound (Z190) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z190) was formed at a vapor-deposition rate of 0.2 nm/s on a glass substrate on which a metal mask with a 2 mm×1 mm opening was disposed. The metal mask was then removed, and 12 nm of silver and magnesium (1/9) was vapor-deposited at a vapor-deposition rate of 0.1 nm/s. A film of silver and magnesium was not formed in a portion where the compound (Z190) was vapor-deposited, and a 2 mm×1 mm transparent region was formed.

### Example 9 Synthesis of Intermediate (S2)

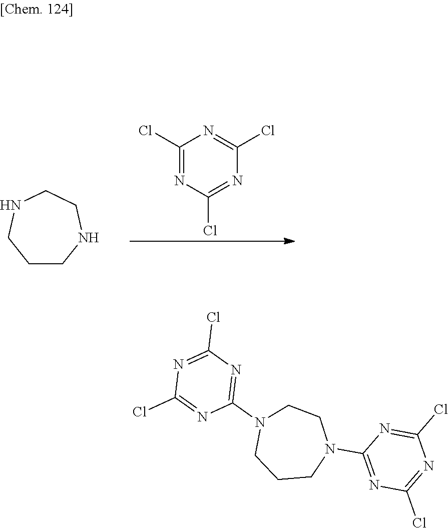 [SMILES: C1CNCCNC1 ; Clc1nc(Cl)nc(Cl)n1 ; Clc1nc(Cl)nc(N2CCCN(c3nc(Cl)nc(Cl)n3)CC2)n1]

In a nitrogen atmosphere, sodium hydroxide (15.0 mmol) and homopiperazine (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. The resulting white solid was collected by filtration and was washed with water and acetone to produce a white solid target intermediate (S2) (yield: 85%).

The compound was identified by FDMS measurement.
FDMS: 394

### Example 10 Synthesis of Compound (Z204)

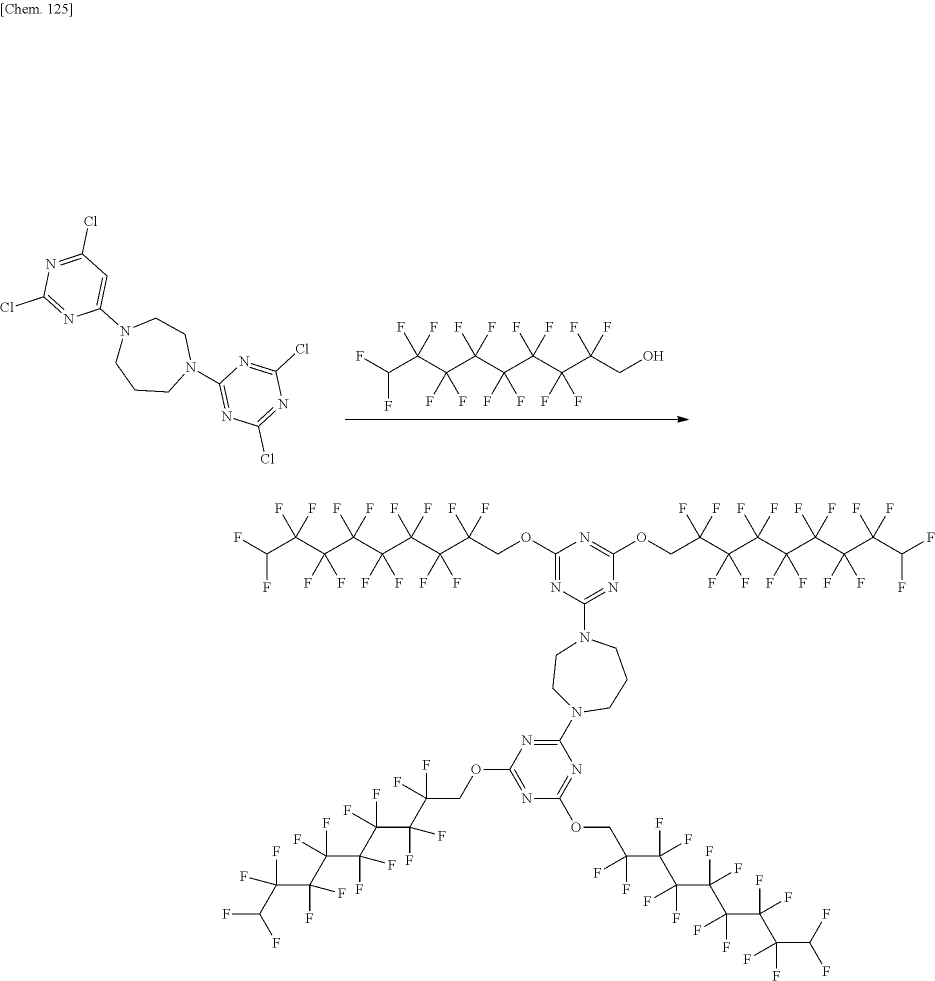 [SMILES: Clc1cc(N2CCCN(c3nc(Cl)nc(Cl)n3)CC2)nc(Cl)n1 ; OCC(F)(F)C(F)(F)C(F)(F)C(F)(F)C(F)(F)C(F)(F)C(F)(F)C(F)F ; FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(N2CCCN(c3nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n3)CC2)n1]

In a nitrogen atmosphere, in the reactor cooled to 0° C., sodium hydride (15.0 mmol) was suspended in tetrahydrofuran (20 ml) and was stirred at 0° C., and 1H, 1H, 9H-hexadecafluoro-1-nonanol (14.0 mmol) was added dropwise to the suspension over 20 minutes. After completion of the dropwise addition, S2 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z204) (yield: 60%).

The compound was identified by FDMS measurement.
FDMS: 1978

### Example 10′ Evaluation of Adhesiveness of Compound (Z204) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 10-nm film of the compound (Z204) was formed at a vapor-deposition rate of 0.2 nm/s on a glass substrate on which a metal mask with a 2 mm×1 mm opening was disposed. The metal mask was then removed, 2 nm of ytterbium and magnesium fluoride (1/1) were vapor-deposited at a vapor-deposition rate of 0.01 nm/s, and 15 nm of silver and magnesium (1/9) was then vapor-deposited at a vapor-deposition rate of 0.1 nm/s. A film of ytterbium, lithium fluoride, silver and magnesium was not formed in a portion where the compound (Z204) was vapor-deposited, and a 2 mm×1 mm transparent region was formed.

### Example 11 Synthesis of Compound (Z205)

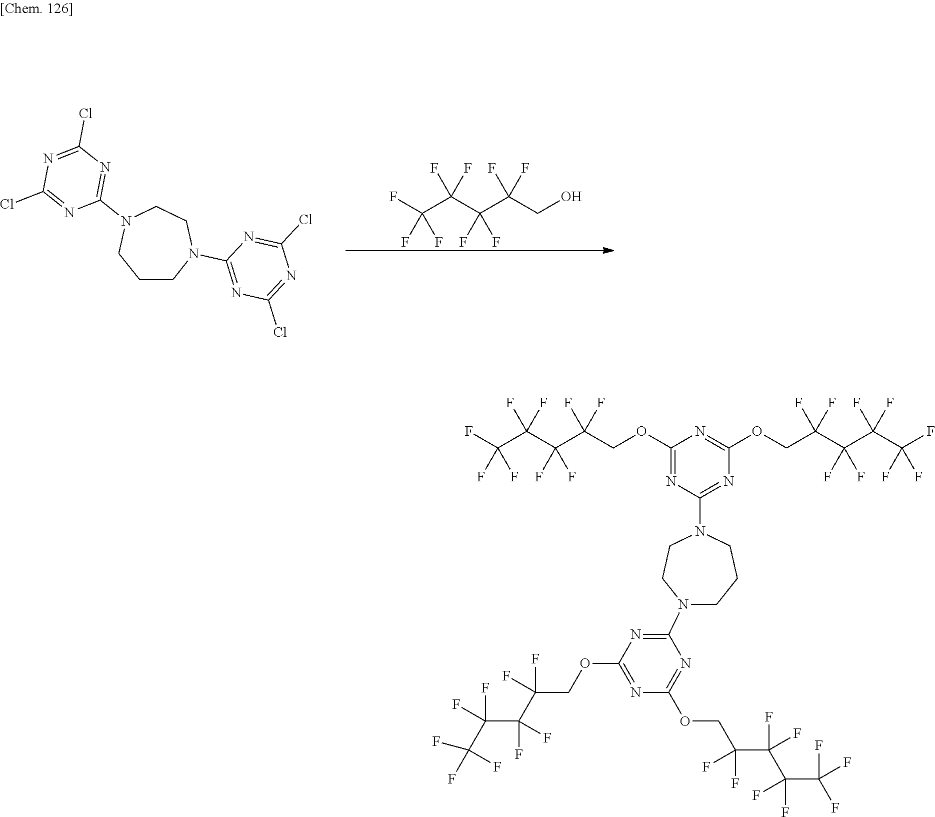 [SMILES: Clc1nc(Cl)nc(N2CCCN(c3nc(Cl)nc(Cl)n3)CC2)n1 ; OCC(F)(F)C(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(N2CCCN(c3nc(OCC(F)(F)C(F)(F)C(F)(F)C(F)(F)F)nc(OCC(F)(F)C(F)(F)C(F)(F)C(F)(F)F)n3)CC2)n1]

In a nitrogen atmosphere, in the reactor cooled to 0° C., sodium hydride (15.0 mmol) was suspended in tetrahydrofuran (20 ml) and was stirred at 0° C., and 1H, 1H-nonafluoro-1-pentanol (14.0 mmol) was added dropwise to the suspension over 20 minutes. After completion of the dropwise addition, S2 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z205) (yield: 72%).

The compound was identified by FDMS measurement.
FDMS: 1250

### Example 11′ Evaluation of Adhesiveness of Compound (Z205) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z205) was formed at a vapor-deposition rate of 0.2 nm/s on a glass substrate on which a metal mask with a 2 mm×1 mm opening was disposed. 20 nm of silver and magnesium (1/9) was then vapor-deposited at a vapor-deposition rate of 0.1 nm/s. A film of silver and magnesium was not formed in a portion where the compound (Z205) was vapor-deposited, and a 2 mm×1 mm transparent region was formed.

### Example 12 Synthesis of Compound (Z206)

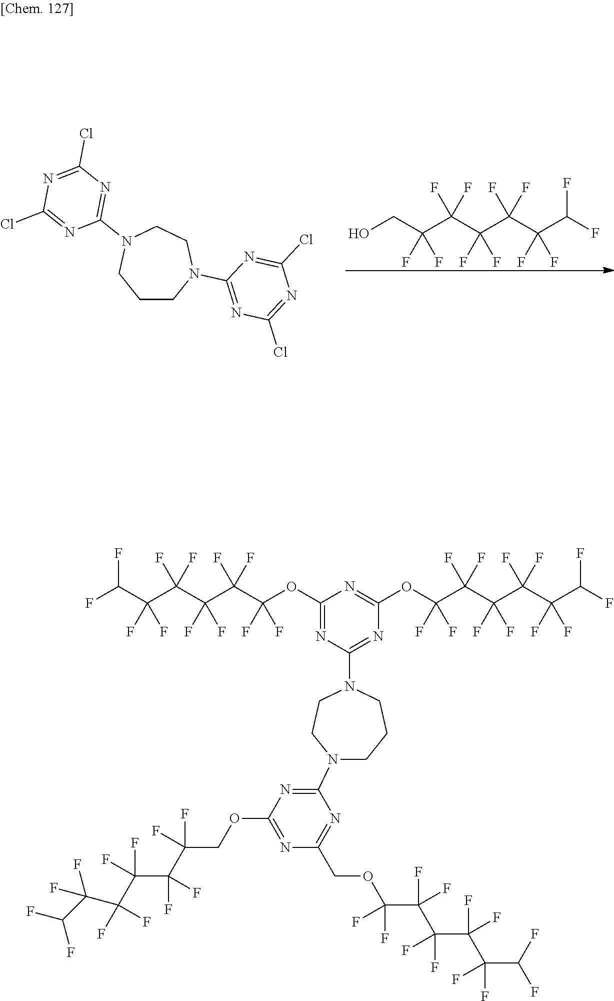 [SMILES: Clc1nc(Cl)nc(N2CCCN(c3nc(Cl)nc(Cl)n3)CC2)n1 ; OCC(F)(F)C(F)(F)C(F)(F)C(F)(F)C(F)(F)C(F)F ; FC(F)C(F)(F)C(F)(F)C(F)(F)C(F)(F)C(F)(F)COc1nc(COC(F)(F)C(F)(F)C(F)(F)C(F)(F)C(F)(F)C(F)F)nc(N2CCCN(c3nc(OC(F)(F)C(F)(F)C(F)(F)C(F)(F)C(F)(F)C(F)F)nc(OC(F)(F)C(F)(F)C(F)(F)C(F)(F)C(F)(F)C(F)F)n3)CC2)n1]

In a nitrogen atmosphere, in the reactor cooled to 0° C., sodium hydride (15.0 mmol) was suspended in tetrahydrofuran (20 ml) and was stirred at 0° C., and 1H, 1H, 7H-dodecafluoro-1-heptanol (14.0 mmol) was added dropwise to the suspension over 20 minutes. After completion of the dropwise addition, S2 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z206) (yield: 78%).

The compound was identified by FDMS measurement.
FDMS: 1578

### Example 12′ Evaluation of Adhesiveness of Compound (Z206) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z206) was formed at a vapor-deposition rate of 0.2 nm/s on a glass substrate on which a metal mask with a 2 mm×1 mm opening was disposed. The metal mask was then removed, 2 nm of ytterbium and magnesium fluoride (1/1) were vapor-deposited at a vapor-deposition rate of 0.01 nm/s, and 15 nm of silver and magnesium (1/1) was then vapor-deposited at a vapor-deposition rate of 0.1 nm/s. A film of ytterbium, lithium fluoride, silver and magnesium was not formed in a portion where the compound (Z206) was vapor-deposited, and a 2 mm×1 mm transparent region was formed.

### Example 13 Synthesis of Compound (Z207)

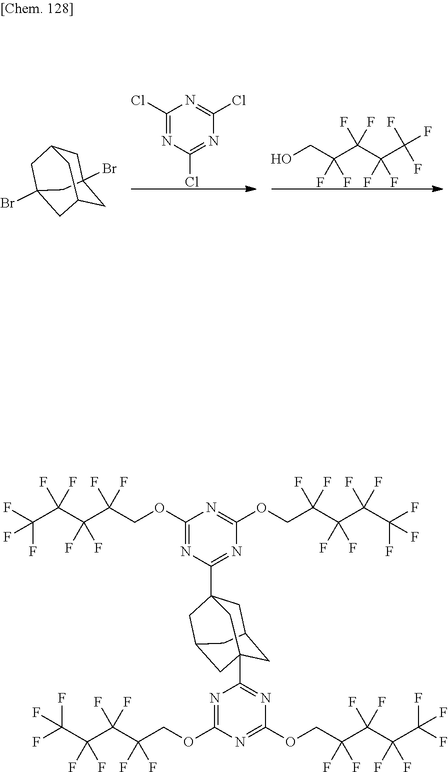 [SMILES: BrC12CC3CC(C1)CC(Br)(C3)C2 ; Clc1nc(Cl)nc(Cl)n1 ; OCC(F)(F)C(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(C23CC4CC(C2)CC(c2nc(OCC(F)(F)C(F)(F)C(F)(F)C(F)(F)F)nc(OCC(F)(F)C(F)(F)C(F)(F)C(F)(F)F)n2)(C4)C3)n1]

In a nitrogen atmosphere, one drop of 1,2-dibromoethane was added to a solution of magnesium (15.0 mmol) suspended in tetrahydrofuran (20 ml), and dibromoadamantane (7.0 mmol) dissolved in tetrahydrofuran (20 ml) was added dropwise over 20 minutes. The dropwise addition was followed by stirring at 60° C. for 3 hours. The temperature was then returned to room temperature, and cyanuric chloride (15.0 mmol) dissolved in tetrahydrofuran (20 ml) was added dropwise over 20 minutes. The dropwise addition was followed by stirring at 60° C. for 3 hours. Next, in the reactor cooled to 0° C., in a nitrogen atmosphere, sodium hydride (32.0 mmol) was suspended in tetrahydrofuran (20 ml) and was stirred at 0° C., and 1H, 1H-nonafluoro-1-pentanol (30.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z207) (yield: 22%).

The compound was identified by FDMS measurement.
FDMS: 1286

### Example 13′ Evaluation of Adhesiveness of Compound (Z207) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z207) was formed at a vapor-deposition rate of 0.2 nm/s on a glass substrate on which a metal mask with a 2 mm×1 mm opening was disposed. The metal mask was then removed, and 5 nm of copper was vapor-deposited at a vapor-deposition rate of 0.1 nm/s. A film of copper was not formed in a portion where the compound (Z207) was vapor-deposited, and a 2 mm×1 mm transparent region was formed.

### Example 14 Synthesis of Compound (Z208)

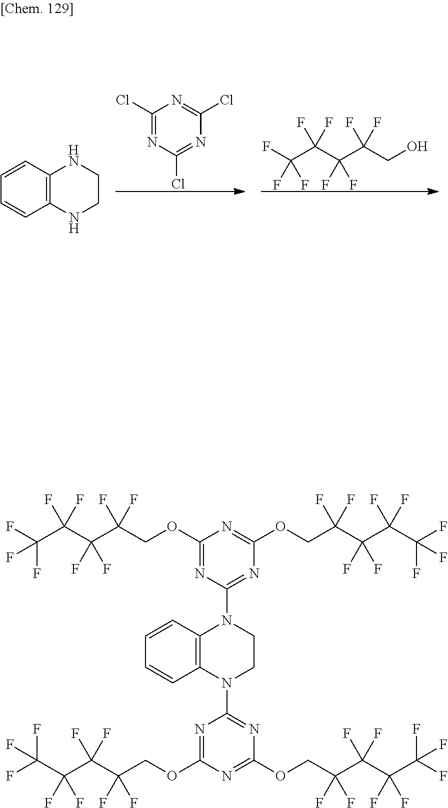 [SMILES: c1ccc2c(c1)NCCN2 ; Clc1nc(Cl)nc(Cl)n1 ; OCC(F)(F)C(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(N2CCN(c3nc(OCC(F)(F)C(F)(F)C(F)(F)C(F)(F)F)nc(OCC(F)(F)C(F)(F)C(F)(F)C(F)(F)F)n3)c3ccccc32)n1]

In a nitrogen atmosphere, sodium hydroxide (15.0 mmol) and 5,6,7,8-tetrahydroquinoxaline (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S3. Subsequently, in a nitrogen atmosphere, in a reactor cooled to 0° C., sodium hydride (15.0 mmol) was suspended in tetrahydrofuran (20 ml) and was stirred at 0° C., and 1H, 1H-nonafluoro-1-pentanol (14.0 mmol) was added dropwise to the suspension over 20 minutes. After completion of the dropwise addition, S3 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z208) (yield: 62%).

The compound was identified by FDMS measurement.
FDMS: 1284

### Example 14′ Evaluation of Adhesiveness of Compound (Z208) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z208) was formed at a vapor-deposition rate of 0.2 nm/s on a glass substrate on which a metal mask with a 2 mm×1 mm opening was disposed. The metal mask was then removed, and 7 nm of gold was vapor-deposited at a vapor-deposition rate of 0.2 nm/s. A film of gold was not formed in a portion where the compound (Z208) was vapor-deposited, and a 2 mm×1 mm transparent region was formed.

### Example 15 Synthesis of Compound (Z209)

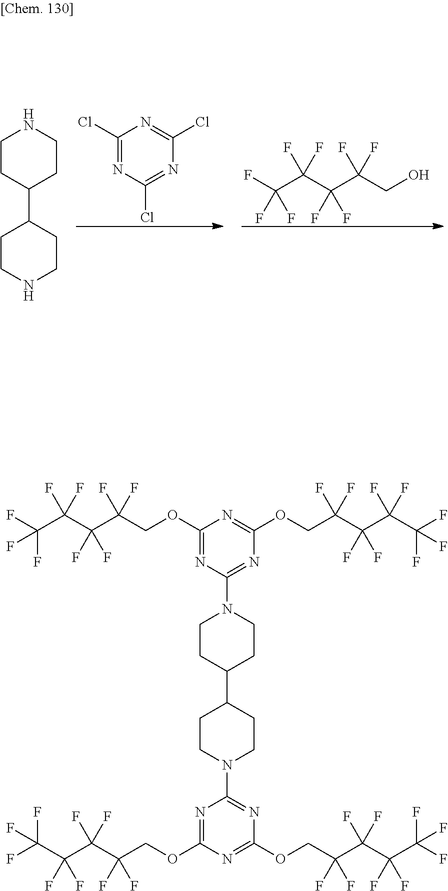

In a nitrogen atmosphere, sodium hydroxide (15.0 mmol) and 4,4′-bipiperidine (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S4. Subsequently, in a nitrogen atmosphere, in a reactor cooled to 0° C., sodium hydride (15.0 mmol) was suspended in tetrahydrofuran (20 ml) and was stirred at 0° C., and 1H, 1H-nonafluoro-1-pentanol (14.0 mmol) was added dropwise to the suspension over 20 minutes. After completion of the dropwise addition, S4 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z209) (yield: 68%).

The compound was identified by FDMS measurement.
FDMS: 1318

### Example 15′ Evaluation of Adhesiveness of Compound (Z209) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z209) was formed at a vapor-deposition rate of 0.2 nm/s on a glass substrate on which a metal mask with a 2 mm×1 mm opening was disposed. The metal mask was then removed, and 10 nm of aluminum was vapor-deposited at a vapor-deposition rate of 0.2 nm/s. A film of aluminum was not formed in a portion where the compound (Z209) was vapor-deposited, and a 2 mm×1 mm transparent region was formed.

### Example 16 Synthesis of Compound (Z210)

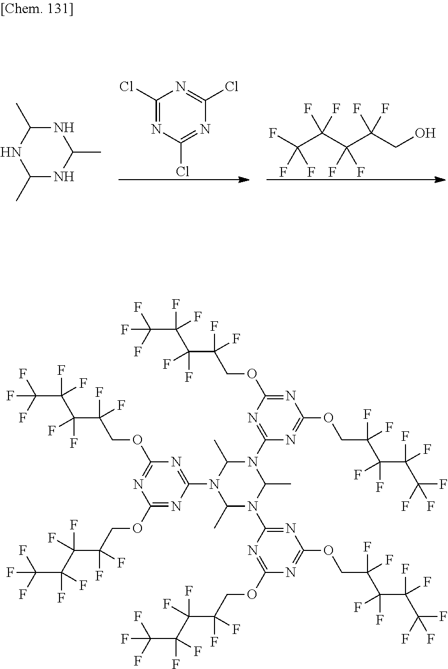

In a nitrogen atmosphere, sodium hydroxide (21.0 mmol) and 2,4,6-trimethylhexahydro-1,3,5-triazine trihydrate (7.0 mmol) were stirred in H₂O (50 ml) at 25° C. for 1 hour and were added dropwise to a tetrahydrofuran (50 ml) solution of cyanuric chloride (21.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S5. Subsequently, in a nitrogen atmosphere, in a reactor cooled to 0° C., sodium hydride (22.0 mmol) was suspended in tetrahydrofuran (20 ml) and was stirred at 0° C., and 1H, 1H-nonafluoro-1-pentanol (20.0 mmol) was added dropwise to the suspension over 20 minutes. After completion of the dropwise addition, S5 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z210) (yield: 15%).

The compound was identified by FDMS measurement.
FDMS: 1854

### Example 16′ Evaluation of Adhesiveness of Compound (Z210) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z210) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z210) was vapor-deposited, and a 2 mm×1 mm transparent region was formed.

### Example 17 Synthesis of Compound (Z211)

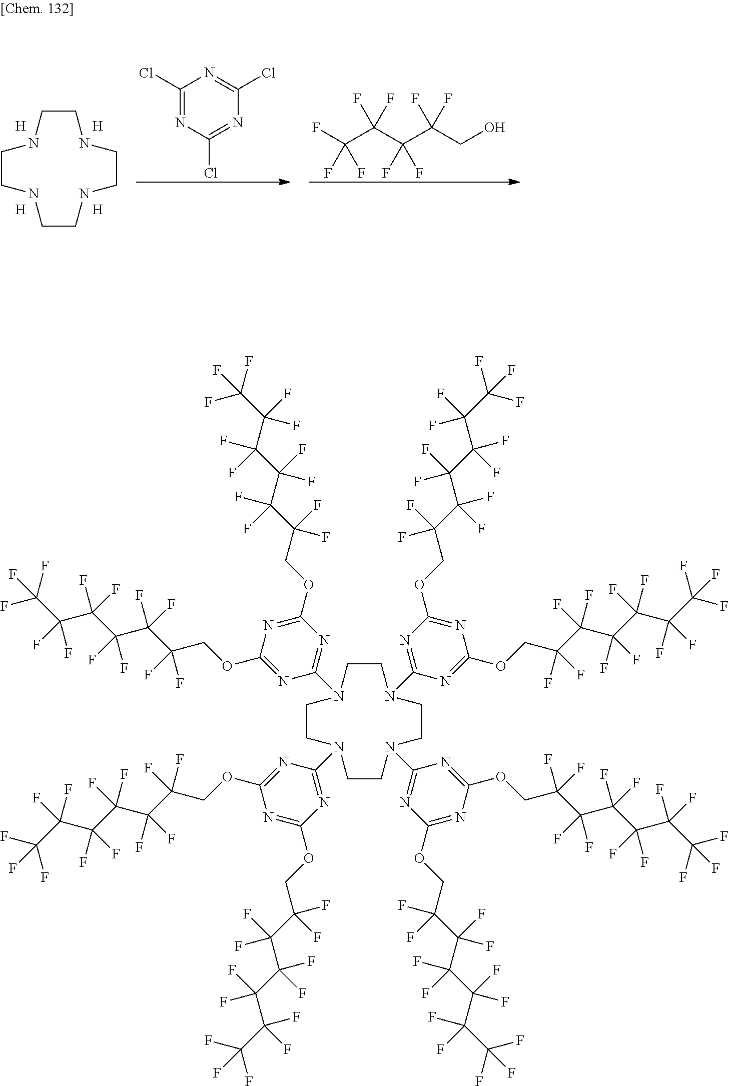

In a nitrogen atmosphere, sodium hydroxide (28.0 mmol) and 1,4,7,10-tetraazacyclododecane (7.0 mmol) were stirred in H₂O (50 ml) at 25° C. for 1 hour and were added dropwise to a tetrahydrofuran (80 ml) solution of cyanuric chloride (28.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S6. Subsequently, in a nitrogen atmosphere, in a reactor cooled to 0° C., sodium hydride (28.0 mmol) was suspended in tetrahydrofuran (20 ml) and was stirred at 0° C., and 1H, 1H-nonafluoro-1-pentanol (26.0 mmol) was added dropwise to the suspension over 20 minutes. After completion of the dropwise addition, S6 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z211) (yield: 11%).

The compound was identified by FDMS measurement.
FDMS: 3272

### Example 17′ Evaluation of Adhesiveness of Compound (Z211) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z211) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z211) was vapor-deposited, and a 2 mm×1 mm transparent region was formed.

### Example 18 Synthesis of Compound (Z212)

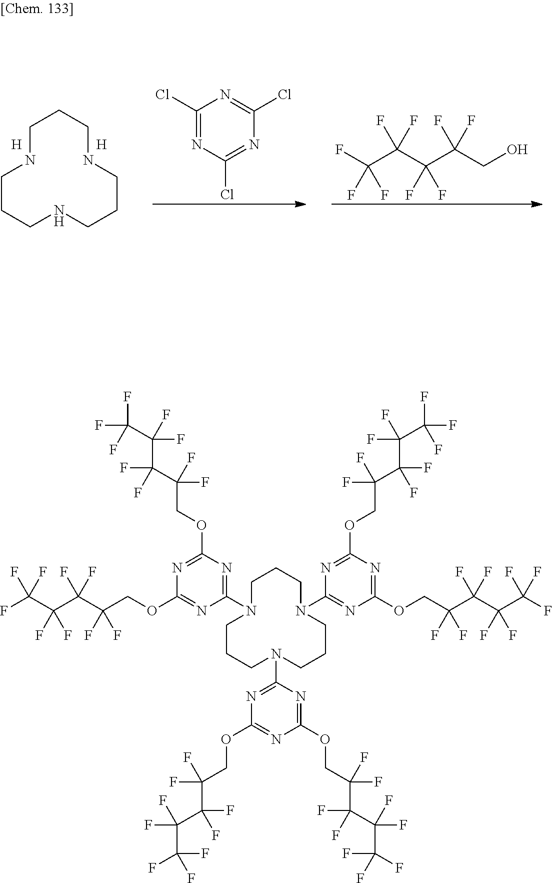

In a nitrogen atmosphere, sodium hydroxide (21.0 mmol) and 1,5,9-triazacyclododecane (7.0 mmol) were stirred in H₂O (50 ml) at 25° C. for 1 hour and were added dropwise to a tetrahydrofuran (50 ml) solution of cyanuric chloride (21.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S7. Subsequently, in a nitrogen atmosphere, in a reactor cooled to 0° C., sodium hydride (22.0 mmol) was suspended in tetrahydrofuran (20 ml) and was stirred at 0° C., and 1H, 1H-nonafluoro-1-pentanol (20.0 mmol) was added dropwise to the suspension over 20 minutes. After completion of the dropwise addition, S7 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z212) (yield: 15%).

The compound was identified by FDMS measurement.
FDMS: 1896

### Example 18′ Evaluation of Adhesiveness of Compound (Z212) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z212) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z212) was vapor-deposited, and a 2 mm×1 mm transparent region was formed.

### Example 19 Synthesis of Compound (Z213)

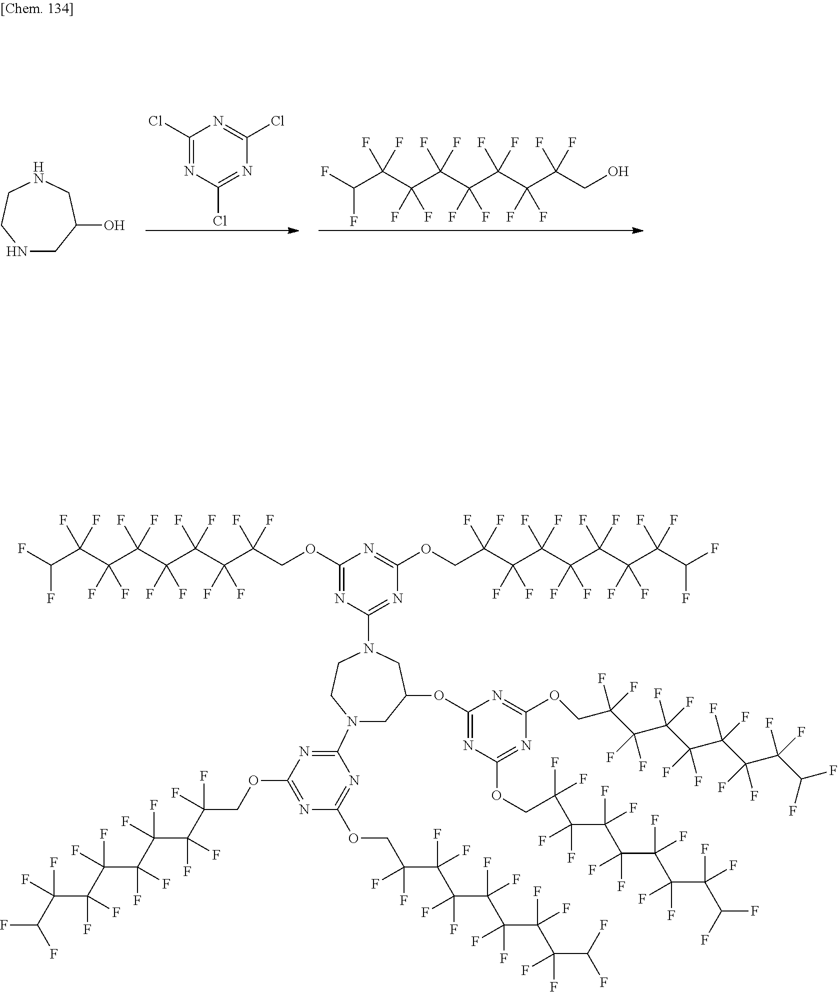 [SMILES: OC1CNCCNC1 ; Clc1nc(Cl)nc(Cl)n1 ; OCC(F)(F)C(F)(F)C(F)(F)C(F)(F)C(F)(F)C(F)(F)C(F)(F)C(F)F ; FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(OC2CN(c3nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n3)CCN(c3nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n3)C2)n1]

In a nitrogen atmosphere, sodium hydroxide (21.0 mmol) and 3-hydroxyhomopiperazine (7.0 mmol) were stirred in H₂O (50 ml) at 25° C. for 1 hour and were added dropwise to a tetrahydrofuran (50 ml) solution of cyanuric chloride (21.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S8. Subsequently, in a nitrogen atmosphere, in a reactor cooled to 0° C., sodium hydride (22.0 mmol) was suspended in tetrahydrofuran (20 ml) and was stirred at 0° C., and 1H, 1H, 9H-hexadecafluoro-1-nonanol (20.0 mmol) was added dropwise to the suspension over 20 minutes. After completion of the dropwise addition, S8 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z213) (yield: 13%).

The compound was identified by FDMS measurement.
FDMS: 2933

### Example 19′ Evaluation of Adhesiveness of Compound (Z213) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z213) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z213) was vapor-deposited, and a 2 mm×1 mm transparent region was formed.

### Example 20 Synthesis of Compound (Z214)

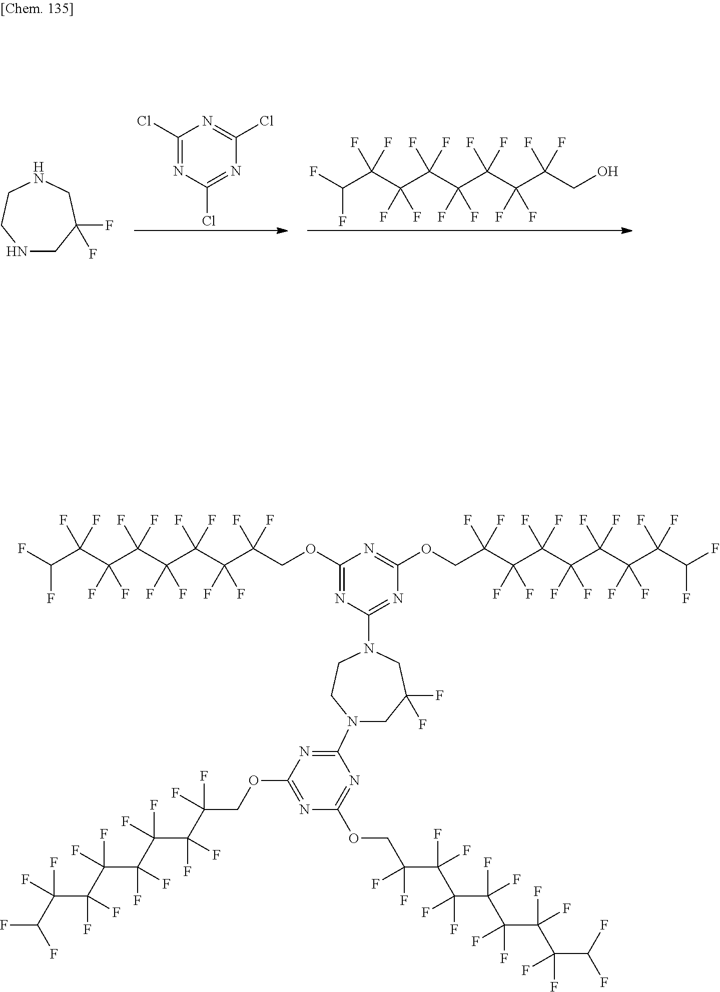 [SMILES: FC1(F)CNCCNC1 ; Clc1nc(Cl)nc(Cl)n1 ; OCC(F)(F)C(F)(F)C(F)(F)C(F)(F)C(F)(F)C(F)(F)C(F)(F)C(F)F ; FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(N2CCN(c3nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n3)CC(F)(F)C2)n1]

In a nitrogen atmosphere, sodium hydroxide (15.0 mmol) and 3,3-difluoro-homopiperazine (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S9. Subsequently, in a nitrogen atmosphere, in a reactor cooled to 0° C., sodium hydride (15.0 mmol) was suspended in tetrahydrofuran (20 ml) and was stirred at 0° C., and 1H, 1H, 9H-hexadecafluoro-1-nonanol (14.0 mmol) was added dropwise to the suspension over 20 minutes. After completion of the dropwise addition, S9 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z214) (yield: 42%).

The compound was identified by FDMS measurement.
FDMS: 2014

### Example 20′ Evaluation of Adhesiveness of Compound (Z214) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z214) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z214) was vapor-deposited, and a 2 mm×1 mm transparent region was formed.

### Example 21 Synthesis of Compound (Z215)

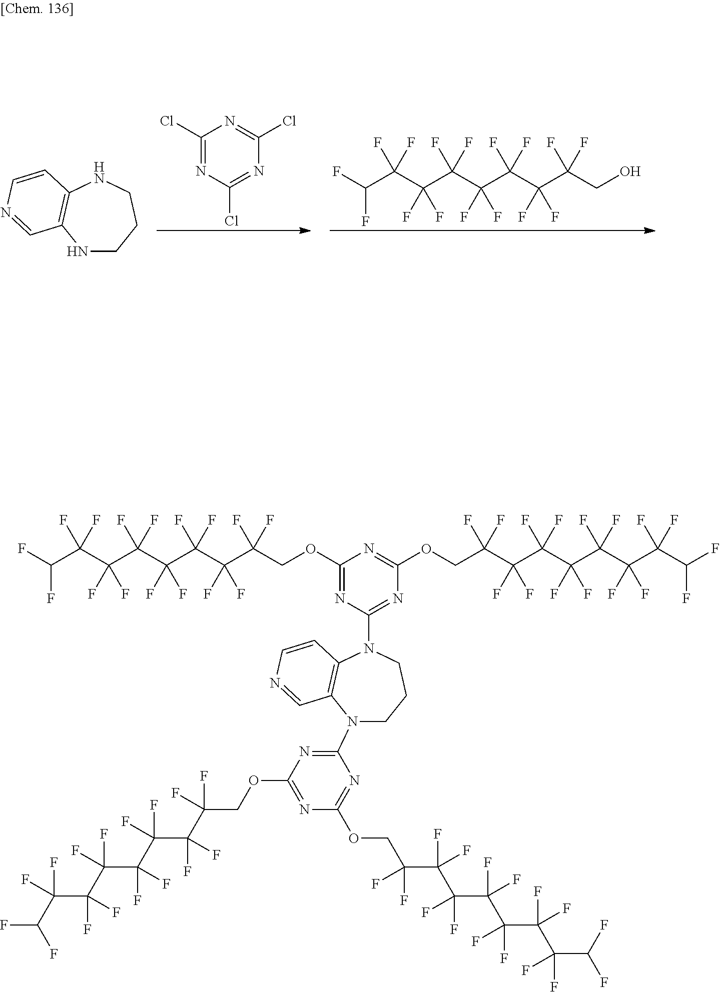 [SMILES: c1cc2c(cn1)NCCCN2 ; Clc1nc(Cl)nc(Cl)n1 ; OCC(F)(F)C(F)(F)C(F)(F)C(F)(F)C(F)(F)C(F)(F)C(F)(F)C(F)F ; FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(N2CCCN(c3nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n3)c3cnccc32)n1]

In a nitrogen atmosphere, sodium hydroxide (15.0 mmol) and 2,3,4,5-tetrahydro-1H-pyridolo[3,4-b][1,4]diazepine (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S10. Subsequently, in a nitrogen atmosphere, in a reactor cooled to 0° C., sodium hydride (15.0 mmol) was suspended in tetrahydrofuran (20 ml) and was stirred at 0° C., and 1H, 1H, 9H-hexadecafluoro-1-nonanol (14.0 mmol) was added dropwise to the suspension over 20 minutes. After completion of the dropwise addition, S10 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z215) (yield: 26%).

The compound was identified by FDMS measurement.
FDMS: 2027

### Example 21′ Evaluation of Adhesiveness of Compound (Z215) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z215) was formed at a vapor-deposition rate of 0.2 nm/s on a glass substrate on which a metal mask with a 2 mm×1 mm opening was disposed. The metal mask was then removed, and 10 nm of magnesium was vapor-deposited at a vapor-deposition rate of 0.2 nm/s. A film of magnesium was not formed in a portion where the compound (Z215) was vapor-deposited, and a 2 mm×1 mm transparent region was formed.

### Example 22 Synthesis of Compound (Z216)

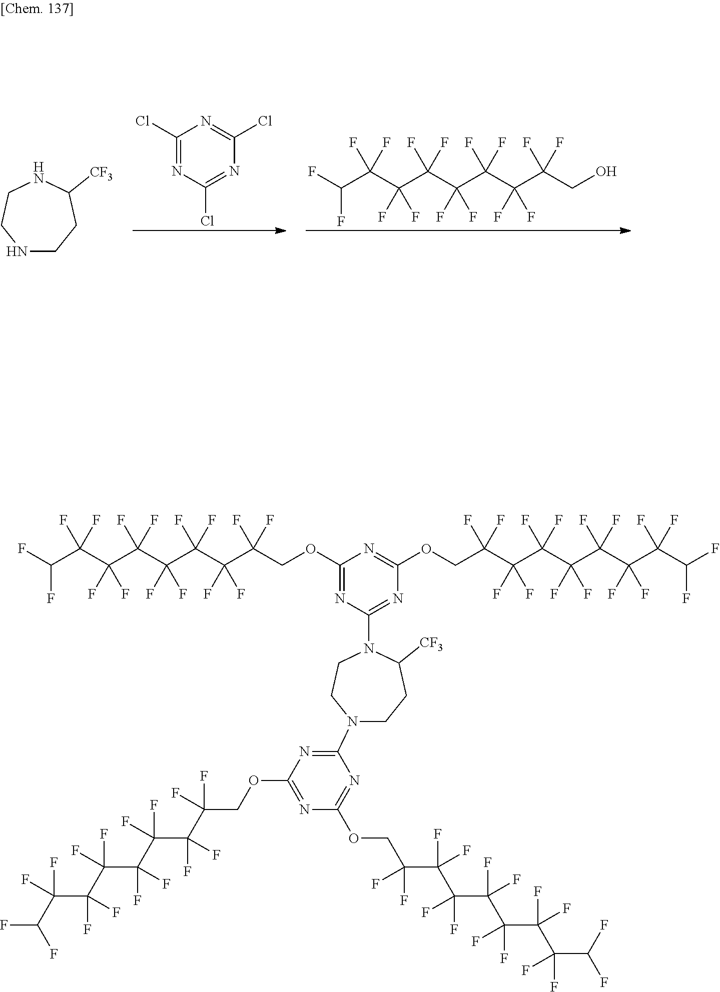 [SMILES: FC(F)(F)C1CCNCCN1 ; Clc1nc(Cl)nc(Cl)n1 ; OCC(F)(F)C(F)(F)C(F)(F)C(F)(F)C(F)(F)C(F)(F)C(F)(F)C(F)F ; FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(N2CCC(C(F)(F)F)N(c3nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n3)CC2)n1]

In a nitrogen atmosphere, sodium hydroxide (15.0 mmol) and 2-trifluoromethyl-homopiperazine (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S11. Subsequently, in a nitrogen atmosphere, in a reactor cooled to 0° C., sodium hydride (15.0 mmol) was suspended in tetrahydrofuran (20 ml) and was stirred at 0° C., and 1H, 1H, 9H-hexadecafluoro-1-nonanol (14.0 mmol) was added dropwise to the suspension over 20 minutes. After completion of the dropwise addition, S11 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z216) (yield: 35%).

The compound was identified by FDMS measurement.
FDMS: 2046

### Example 22′ Evaluation of Adhesiveness of Compound (Z216) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 30-nm film of the compound (Z216) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z216) was vapor-deposited, and a 2 mm×1 mm transparent region was formed.

### Example 23 Synthesis of Compound (Z217)

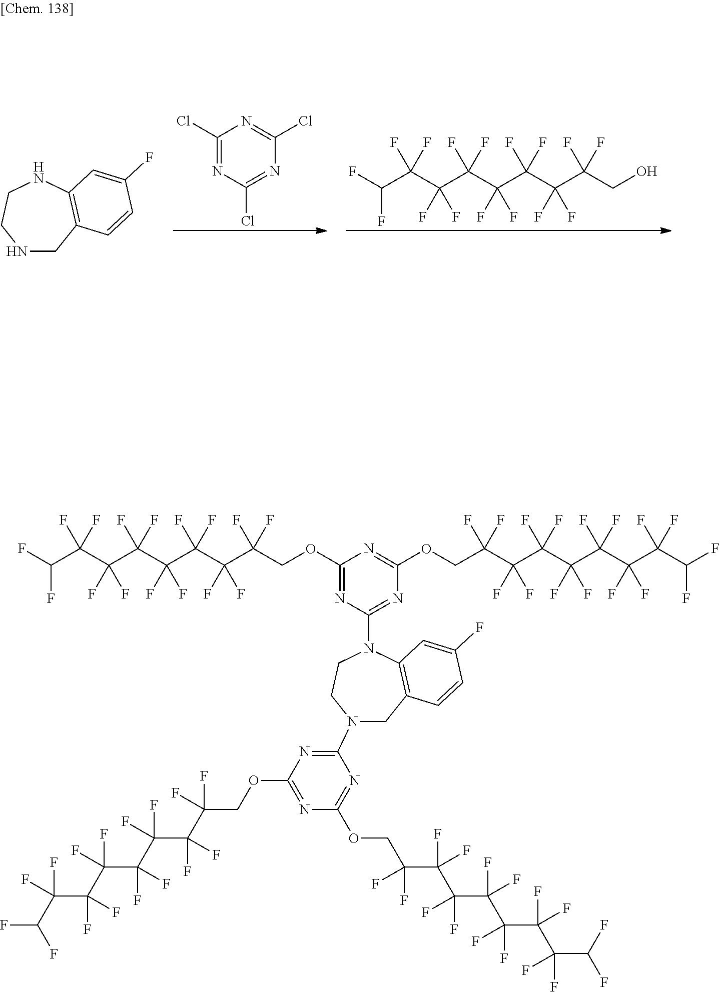 [SMILES: Fc1ccc2c(c1)NCCNC2 ; Clc1nc(Cl)nc(Cl)n1 ; OCC(F)(F)C(F)(F)C(F)(F)C(F)(F)C(F)(F)C(F)(F)C(F)(F)C(F)F ; Fc1ccc2c(c1)N(c1nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n1)CCN(c1nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n1)C2]

In a nitrogen atmosphere, sodium hydroxide (15.0 mmol) and 8-fluoro-2,3,4,5-tetrahydro-1H-1,4-benzodiazepine (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S12. Subsequently, in a nitrogen atmosphere, in a reactor cooled to 0° C., sodium hydride (15.0 mmol) was suspended in tetrahydrofuran (20 ml) and was stirred at 0° C., and 1H, 1H, 9H-hexadecafluoro-1-nonanol (14.0 mmol) was added dropwise to the suspension over 20 minutes. After completion of the dropwise addition, S12 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z217) (yield: 54%).

The compound was identified by FDMS measurement.
FDMS: 2044

### Example 23′ Evaluation of Adhesiveness of Compound (Z217) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z217) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z217) was vapor-deposited, and a 2 mm×1 mm transparent region was formed.

### Example 24 Synthesis of Compound (Z218)

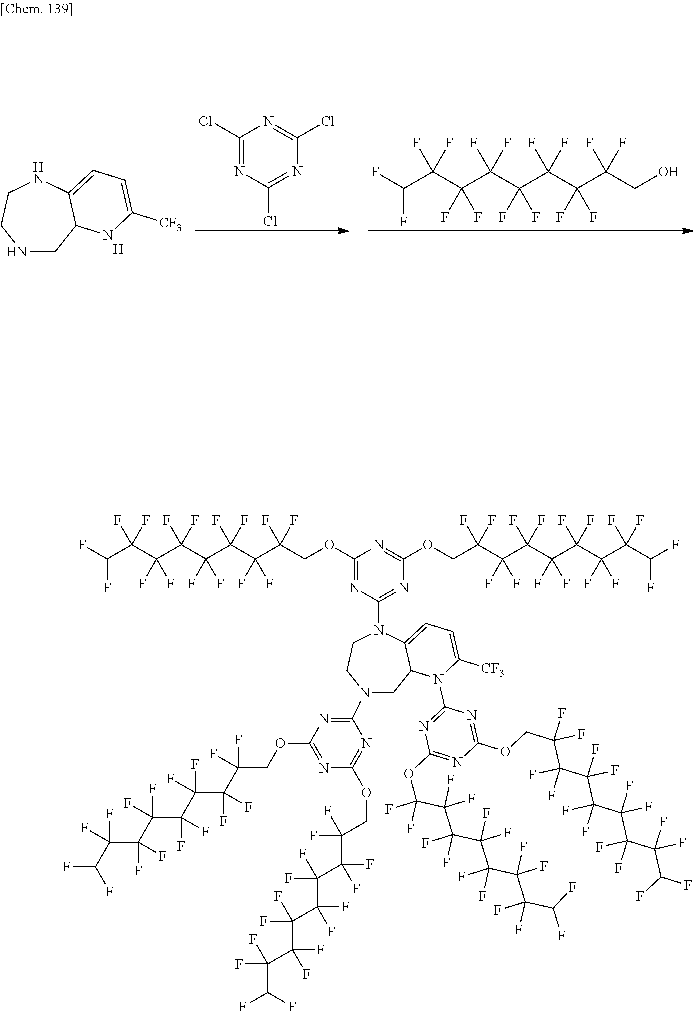

In a nitrogen atmosphere, sodium hydroxide (21.0 mmol) and 1H-pyrido[2,3, e]-1,4-diazepine, 2,3,4,5-tetrahydro-8-(trifluoromethyl)-hydrochloride (7.0 mmol) were stirred in H₂O (50 ml) at 25° C. for 1 hour and were added dropwise to a tetrahydrofuran (50 ml) solution of cyanuric chloride (21.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S13. Subsequently, in a nitrogen atmosphere, in a reactor cooled to 0° C., sodium hydride (22.0 mmol) was suspended in tetrahydrofuran (20 ml) and was stirred at 0° C., and 1H, 1H, 9H-hexadecafluoro-1-nonanol (20.0 mmol) was added dropwise to the suspension over 20 minutes. After completion of the dropwise addition, S13 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z218) (yield: 9%).

The compound was identified by FDMS measurement.
FDMS: 3036

### Example 24′ Evaluation of Adhesiveness of Compound (Z218) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z218) was formed at a vapor-deposition rate of 0.2 nm/s on a glass substrate on which a metal mask with a 2 mm×1 mm opening was disposed. The metal mask was then removed, and 25 nm of silver was vapor-deposited at a vapor-deposition rate of 0.2 nm/s. A film of silver was not formed in a portion where the compound (Z218) was vapor-deposited, and a 2 mm×1 mm transparent region was formed.

### Example 25 Synthesis of Compound (Z219)

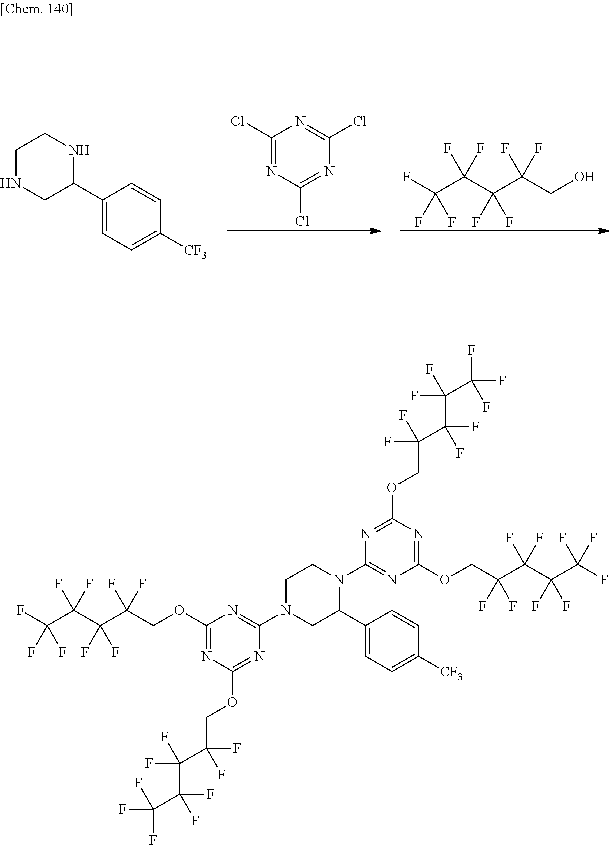 [SMILES: FC(F)(F)c1ccc(C2CNCCN2)cc1 ; Clc1nc(Cl)nc(Cl)n1 ; OCC(F)(F)C(F)(F)C(F)(F)C(F)(F)F ; FC(F)(F)c1ccc(C2CN(c3nc(OCC(F)(F)C(F)(F)C(F)(F)C(F)(F)F)nc(OCC(F)(F)C(F)(F)C(F)(F)C(F)(F)F)n3)CCN2c2nc(OCC(F)(F)C(F)(F)C(F)(F)C(F)(F)F)nc(OCC(F)(F)C(F)(F)C(F)(F)C(F)(F)F)n2)cc1]

In a nitrogen atmosphere, sodium hydroxide (15.0 mmol) and 2-[4-(trifluoromethyl)phenyl]piperazine (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S14. Subsequently, in a nitrogen atmosphere, in a reactor cooled to 0° C., sodium hydride (15.0 mmol) was suspended in tetrahydrofuran (20 ml) and was stirred at 0° C., and 1H, 1H-nonafluoro-1-pentanol (14.0 mmol) was added dropwise to the suspension over 20 minutes. After completion of the dropwise addition, S14 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z219) (yield: 67%).

The compound was identified by FDMS measurement.
FDMS: 1380

### Example 25′ Evaluation of Adhesiveness of Compound (Z219) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z219) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z219) was vapor-deposited, and a 2 mm×1 mm transparent region was formed.

### Example 26 Synthesis of Compound (Z220)

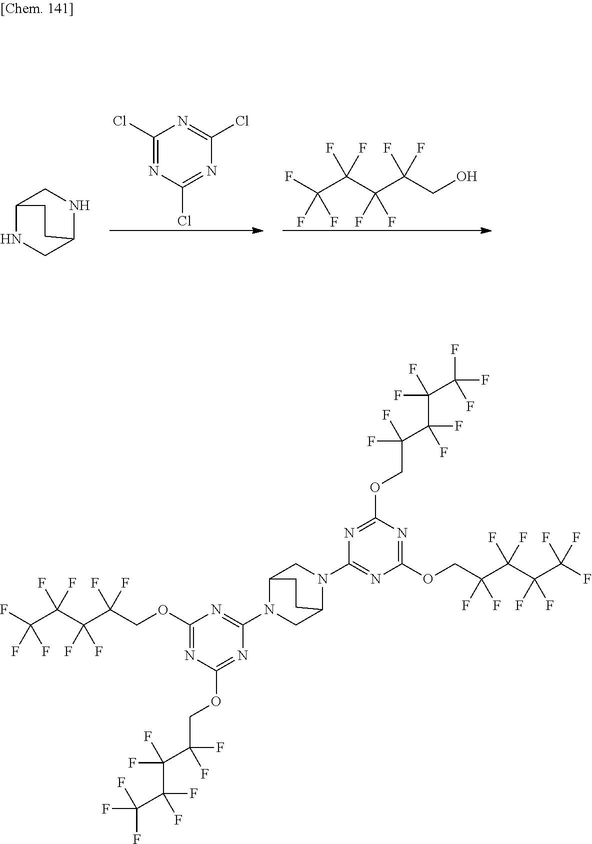 [SMILES: C1CC2CNC1CN2 ; Clc1nc(Cl)nc(Cl)n1 ; OCC(F)(F)C(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(N2CC3CCC2CN3c2nc(OCC(F)(F)C(F)(F)C(F)(F)C(F)(F)F)nc(OCC(F)(F)C(F)(F)C(F)(F)C(F)(F)F)n2)n1]

In a nitrogen atmosphere, sodium hydroxide (15.0 mmol) and 2,5-diazabicyclo[2,2,2]octane (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S15. Subsequently, in a nitrogen atmosphere, in a reactor cooled to 0° C., sodium hydride (15.0 mmol) was suspended in tetrahydrofuran (20 ml) and was stirred at 0° C., and 1H, 1H-nonafluoro-1-pentanol (14.0 mmol) was added dropwise to the suspension over 20 minutes. After completion of the dropwise addition, S15 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z220) (yield: 71%).

The compound was identified by FDMS measurement.
FDMS: 1262

### Example 26′ Evaluation of Adhesiveness of Compound (Z220) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z220) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z220) was vapor-deposited, and a 2 mm×1 mm transparent region was formed.

### Example 27 Synthesis of Compound (Z221)

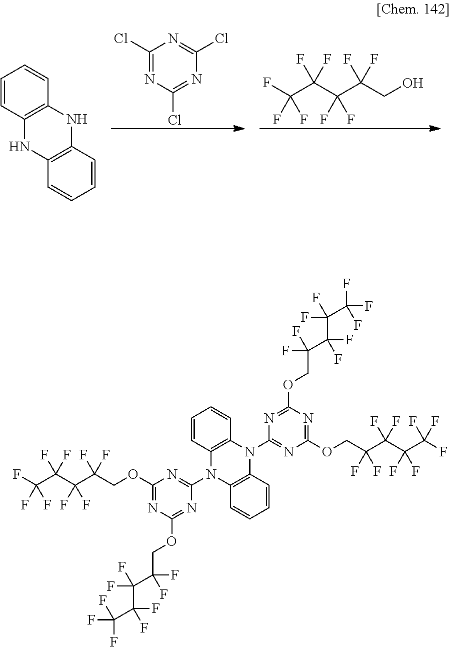 [SMILES: c1ccc2c(c1)Nc1ccccc1N2 ; Clc1nc(Cl)nc(Cl)n1 ; OCC(F)(F)C(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(N2c3ccccc3N(c3nc(OCC(F)(F)C(F)(F)C(F)(F)C(F)(F)F)nc(OCC(F)(F)C(F)(F)C(F)(F)C(F)(F)F)n3)c3ccccc32)n1]

In a nitrogen atmosphere, sodium hydroxide (15.0 mmol) and 5,10-dihydro-phenazine (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S16. Subsequently, in a nitrogen atmosphere, in a reactor cooled to 0° C., sodium hydride (15.0 mmol) was suspended in tetrahydrofuran (20 ml) and was stirred at 0° C., and 1H, 1H-nonafluoro-1-pentanol (14.0 mmol) was added dropwise to the suspension over 20 minutes. After completion of the dropwise addition, S16 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z221) (yield: 19%).

The compound was identified by FDMS measurement.
FDMS: 1332

### Example 27′ Evaluation of Adhesiveness of Compound (Z221) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z221) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z221) was vapor-deposited, and a 2 mm×1 mm transparent region was formed.

### Example 28 Synthesis of Compound (Z222)

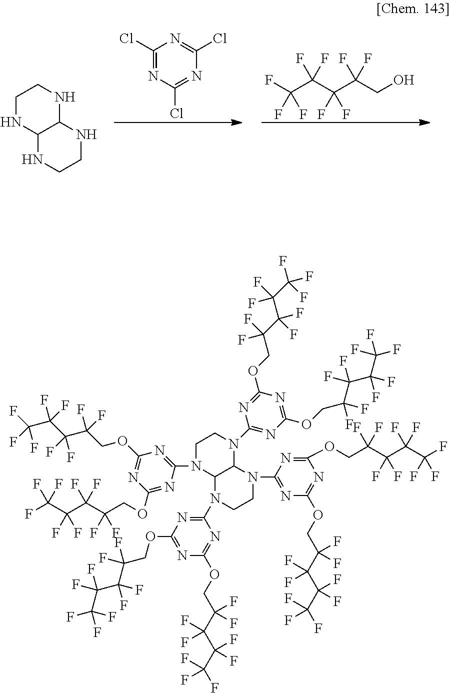 [SMILES: C1CNC2NCCNC2N1 ; Clc1nc(Cl)nc(Cl)n1 ; OCC(F)(F)C(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(N2CCN(c3nc(OCC(F)(F)C(F)(F)C(F)(F)C(F)(F)F)nc(OCC(F)(F)C(F)(F)C(F)(F)C(F)(F)F)n3)C3C2N(c2nc(OCC(F)(F)C(F)(F)C(F)(F)C(F)(F)F)nc(OCC(F)(F)C(F)(F)C(F)(F)C(F)(F)F)n2)CCN3c2nc(OCC(F)(F)C(F)(F)C(F)(F)C(F)(F)F)nc(OCC(F)(F)C(F)(F)C(F)(F)C(F)(F)F)n2)n1]

In a nitrogen atmosphere, sodium hydroxide (29.0 mmol) and decahydropyrazino[2,3-b]pyrazine (7.0 mmol) were stirred in H₂O (50 ml) at 25° C. for 1 hour and were added dropwise to a tetrahydrofuran (80 ml) solution of cyanuric chloride (28.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S17. Subsequently, in a nitrogen atmosphere, in a reactor cooled to 0° C., sodium hydride (28.0 mmol) was suspended in tetrahydrofuran (20 ml) and was stirred at 0° C., and 1H, 1H-nonafluoro-1-pentanol (26.0 mmol) was added dropwise to the suspension over 20 minutes. After completion of the dropwise addition, S17 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z222) (yield: 4%).

The compound was identified by FDMS measurement.
FDMS: 2442

### Example 28′ Evaluation of Adhesiveness of Compound (Z222) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z222) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z222) was vapor-deposited, and a 2 mm×1 mm transparent region was formed.

### Example 29 Synthesis of Compound (Z223)

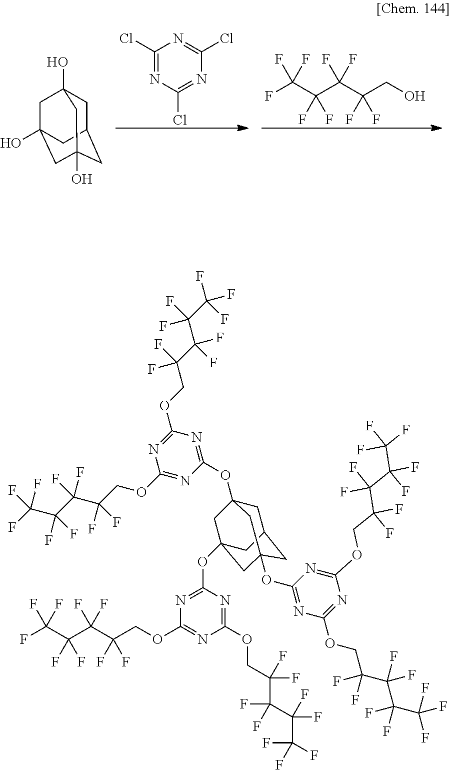 [SMILES: OC12CC3CC(O)(C1)CC(O)(C3)C2 ; Clc1nc(Cl)nc(Cl)n1 ; OCC(F)(F)C(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(OC23CC4CC(Oc5nc(OCC(F)(F)C(F)(F)C(F)(F)C(F)(F)F)nc(OCC(F)(F)C(F)(F)C(F)(F)C(F)(F)F)n5)(C2)CC(Oc2nc(OCC(F)(F)C(F)(F)C(F)(F)C(F)(F)F)nc(OCC(F)(F)C(F)(F)C(F)(F)C(F)(F)F)n2)(C4)C3)n1]

In a nitrogen atmosphere, sodium hydroxide (21.0 mmol) and adamantanetriol (7.0 mmol) were stirred in H₂O (50 ml) at 25° C. for 1 hour and were added dropwise to a tetrahydrofuran (80 ml) solution of cyanuric chloride (21.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S18. Subsequently, in a nitrogen atmosphere, in a reactor cooled to 0° C., sodium hydride (22.0 mmol) was suspended in tetrahydrofuran (20 ml) and was stirred at 0° C., and 1H, 1H-nonafluoro-1-pentanol (20.0 mmol) was added dropwise to the suspension over 20 minutes. After completion of the dropwise addition, S18 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z223) (yield: 31%).

The compound was identified by FDMS measurement.
FDMS: 1909

### Example 29′ Evaluation of Adhesiveness of Compound (Z223) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z223) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z223) was vapor-deposited, and a 2 mm×1 mm transparent region was formed.

### Example 30 Synthesis of Compound (Z224)

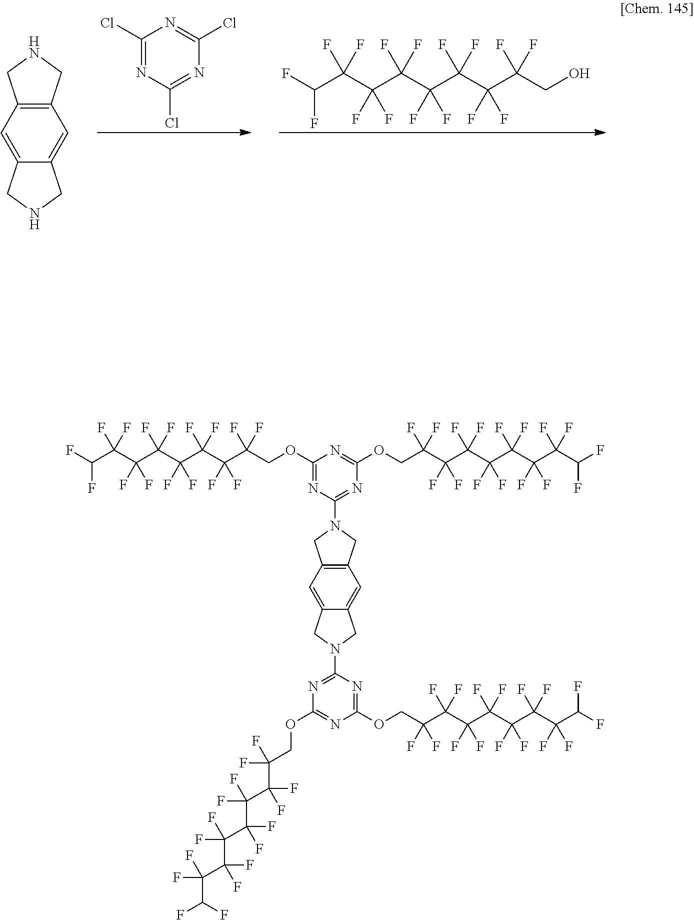 [SMILES: c1c2c(cc3c1CNC3)CNC2 ; Clc1nc(Cl)nc(Cl)n1 ; OCC(F)(F)C(F)(F)C(F)(F)C(F)(F)C(F)(F)C(F)(F)C(F)(F)C(F)F ; FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(N2Cc3cc4c(cc3C2)CN(c2nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n2)C4)n1]

In a nitrogen atmosphere, sodium hydroxide (15.0 mmol) and 1, 2, 3, 5,6,7-hexahydrobenzo[1,2-c: 4,5-c′]dipyrrole (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S19. Subsequently, in a nitrogen atmosphere, in a reactor cooled to 0° C., sodium hydride (15.0 mmol) was suspended in tetrahydrofuran (20 ml) and was stirred at 0° C., and 1H, 1H, 9H-hexadecafluoro-1-nonanol (14.0 mmol) was added dropwise to the suspension over 20 minutes. After completion of the dropwise addition, S19 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z224) (yield: 48%).

The compound was identified by FDMS measurement.
FDMS: 2038

### Example 30′ Evaluation of Adhesiveness of Compound (Z224) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z224) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z224) was vapor-deposited, and a 2 mm×1 mm transparent region was formed.

### Example 31 Synthesis of Compound (Z225)

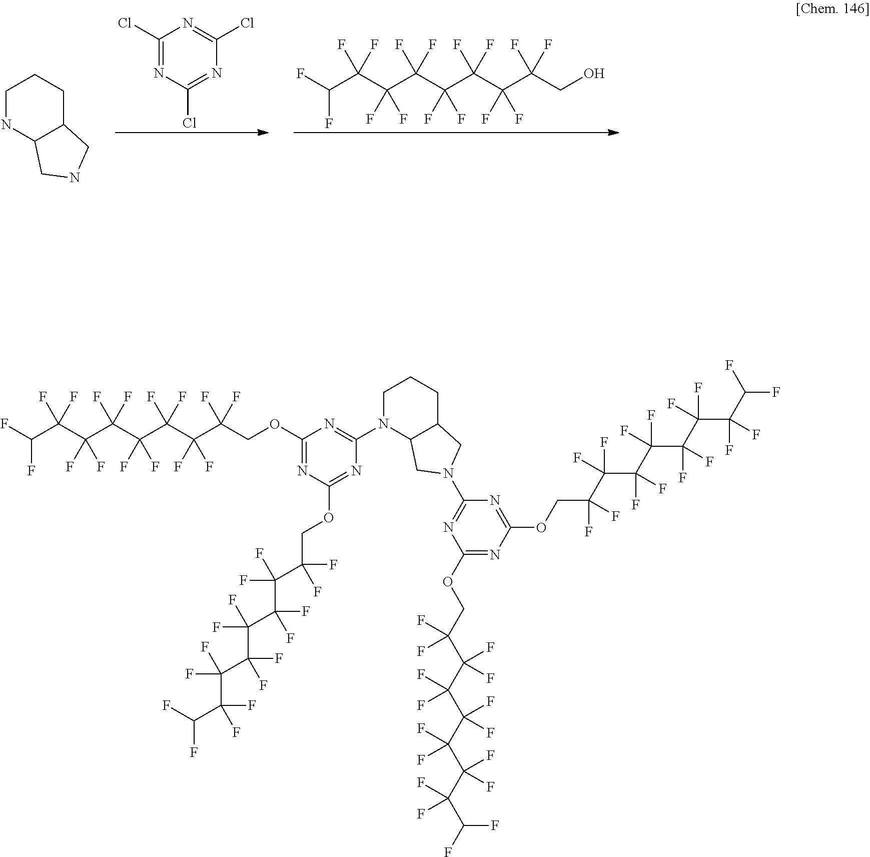 [SMILES: C1CNC2CNCC2C1 ; Clc1nc(Cl)nc(Cl)n1 ; OCC(F)(F)C(F)(F)C(F)(F)C(F)(F)C(F)(F)C(F)(F)C(F)(F)C(F)F ; FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(N2CC3CCCN(c4nc(OCC(F)(F)C(F)(F)C(F)(F)C(F)(F)C(F)(F)C(F)(F)C(F)(F)C(F)F)nc(OCC(F)(F)C(F)(F)C(F)(F)C(F)(F)C(F)(F)C(F)(F)C(F)(F)C(F)F)n4)C3C2)n1]

In a nitrogen atmosphere, sodium hydroxide (15.0 mmol) and octahydro-1H-pyrrolo[3,4-b]pyridine (7.0 mmol) were stirred in H₂O (50 ml) at 25° C. for 1 hour and were added dropwise to a tetrahydrofuran (50 ml) solution of cyanuric chloride (14.0 mmol) at 0° C. over 30 minutes. The dropwise addition was followed by stirring also at 0° C. for 2 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S20. Subsequently, in a nitrogen atmosphere, in a reactor cooled to 0° C., sodium hydride (15.0 mmol) was suspended in tetrahydrofuran (20 ml) and was stirred at 0° C., and 1H, 1H, 9H-hexadecafluoro-1-nonanol (14.0 mmol) was added dropwise to the suspension over 20 minutes. After completion of the dropwise addition, S20 (3.0 mmol) dissolved in tetrahydrofuran (20 ml) was added dropwise over 10 minutes. The dropwise addition was followed by stirring at 25° C. for 5 hours. The suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z225) (yield: 49%).

The compound was identified by FDMS measurement.
FDMS: 2004

### Example 31′ Evaluation of Adhesiveness of Compound (Z225) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z225) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z225) was vapor-deposited, and a 2 mm×1 mm transparent region was formed.

### Example 32 Synthesis of Compound (Z226)

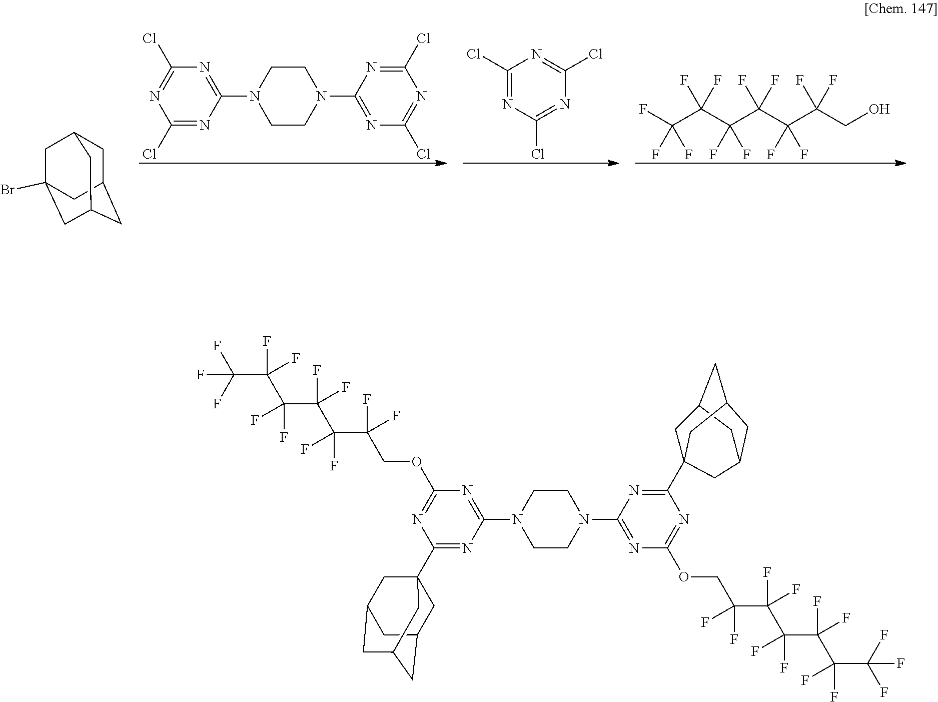 [SMILES: BrC12CC3CC(CC(C3)C1)C2 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)F ; FC(F)(F)C(F)(F)C(F)(F)C(F)(F)C(F)(F)C(F)(F)COc1nc(N2CCN(c3nc(OCC(F)(F)C(F)(F)C(F)(F)C(F)(F)C(F)(F)C(F)(F)F)nc(C45CC6CC(CC(C6)C4)C5)n3)CC2)nc(C23CC4CC(CC(C4)C2)C3)n1]

In a nitrogen atmosphere, one drop of 1,2-dibromoethane was added to a solution of magnesium (15.0 mmol) suspended in tetrahydrofuran (20 ml), and 1-bromoadamantane (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z226) (yield: 22%).

The compound was identified by FDMS measurement.
FDMS: 1208

### Example 32′ Evaluation of Adhesiveness of Compound (Z226) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z226) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z226) was vapor-deposited, and a 2 mm×1 mm transparent region was formed.

### Example 33 Synthesis of Compound (Z227)

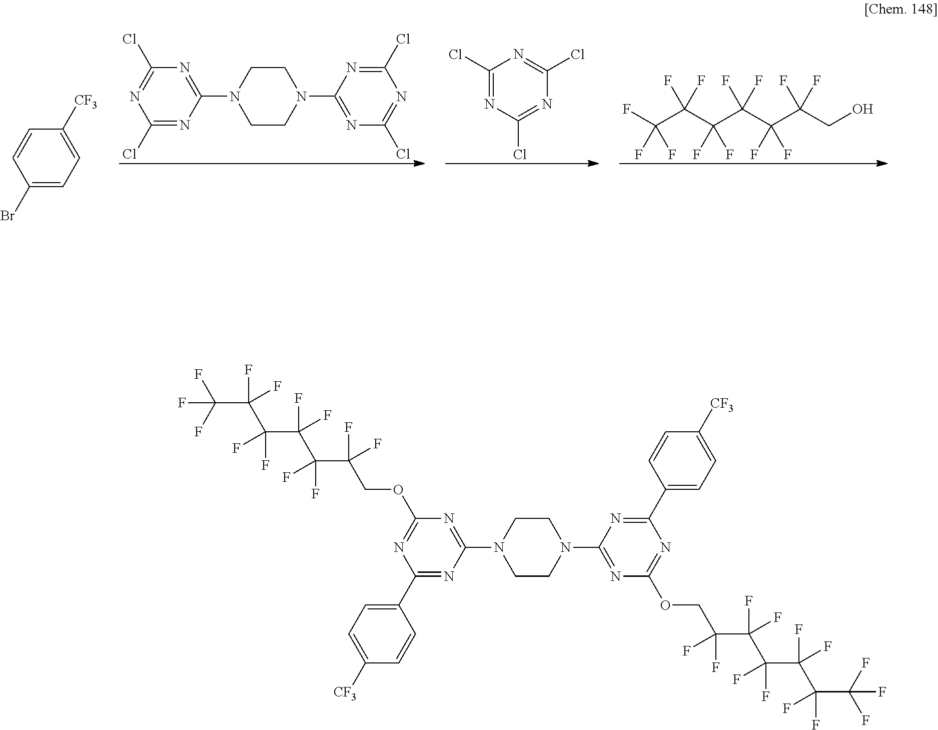 [SMILES: FC(F)(F)c1ccc(Br)cc1 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)F ; FC(F)(F)c1ccc(-c2nc(OCC(F)(F)C(F)(F)C(F)(F)C(F)(F)C(F)(F)C(F)(F)F)nc(N3CCN(c4nc(OCC(F)(F)C(F)(F)C(F)(F)C(F)(F)C(F)(F)C(F)(F)F)nc(-c5ccc(C(F)(F)F)cc5)n4)CC3)n2)cc1]

In a nitrogen atmosphere, one drop of 1,2-dibromoethane was added to a solution of magnesium (15.0 mmol) suspended in tetrahydrofuran (20 ml), and 4-trifluoromethylbromobenzene (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z227) (yield: 32%).

The compound was identified by FDMS measurement.
FDMS: 1228

### Example 33′ Evaluation of Adhesiveness of Compound (Z227) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z227) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z227) was vapor-deposited, and a 2 mm×1 mm transparent region was formed.

### Example 34 Synthesis of Compound (Z228)

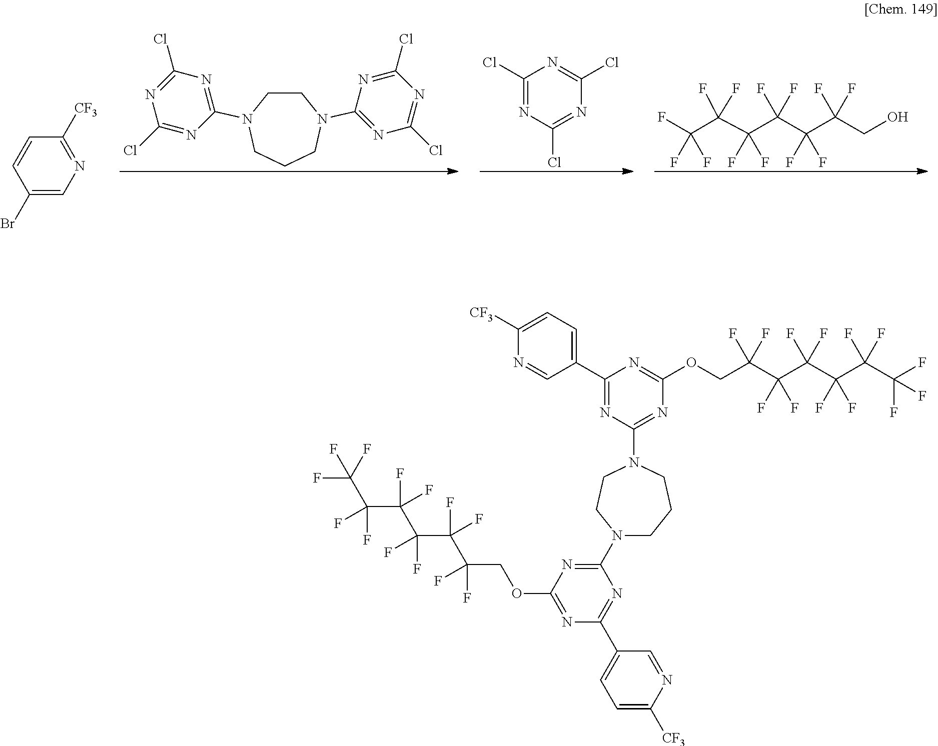 [SMILES: FC(F)(F)c1ccc(Br)cn1 ; Clc1nc(Cl)nc(N2CCCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)F ; FC(F)(F)c1ccc(-c2nc(OCC(F)(F)C(F)(F)C(F)(F)C(F)(F)C(F)(F)C(F)(F)F)nc(N3CCCN(c4nc(OCC(F)(F)C(F)(F)C(F)(F)C(F)(F)C(F)(F)C(F)(F)F)nc(-c5ccc(C(F)(F)F)nc5)n4)CC3)n2)cn1]

In a nitrogen atmosphere, one drop of 1,2-dibromoethane was added to a solution of magnesium (15.0 mmol) suspended in tetrahydrofuran (20 ml), and 2-trifluoromethyl-5-bromopyridine (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z228) (yield: 27%).

The compound was identified by FDMS measurement.
FDMS: 1244

### Example 34′ Evaluation of Adhesiveness of Compound (Z228) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z228) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z228) was vapor-deposited, and a 2 mm×1 mm transparent region was formed.

### Example 35 Synthesis of Compound (Z229)

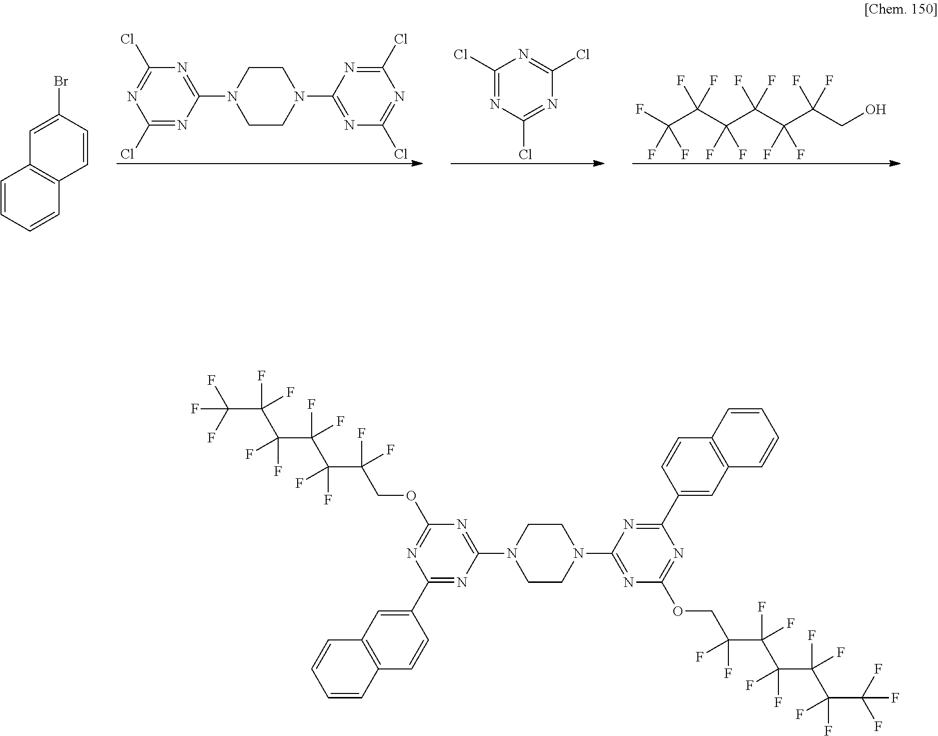

In a nitrogen atmosphere, one drop of 1,2-dibromoethane was added to a solution of magnesium (15.0 mmol) suspended in tetrahydrofuran (20 ml), and 2-bromonaphthalene (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z229) (yield: 57%).

The compound was identified by FDMS measurement.
FDMS: 1192

### Example 35′ Evaluation of Adhesiveness of Compound (Z229) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z229) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z229) was vapor-deposited, and a 2 mm×1 mm transparent region was formed.

### Example 36 Synthesis of Compound (Z230)

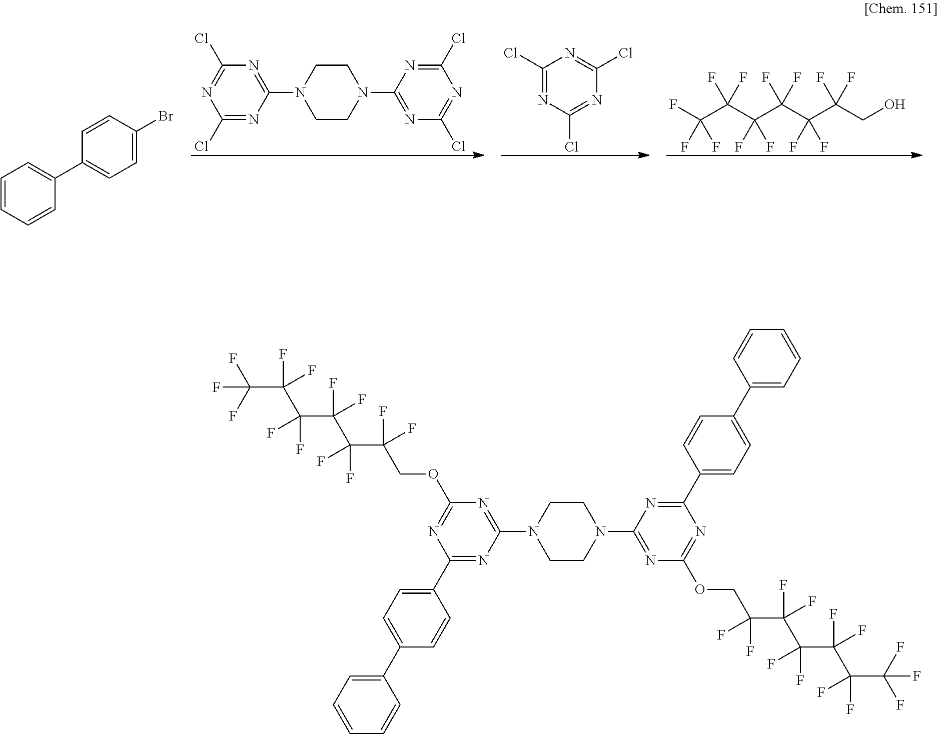 [SMILES: Brc1ccc(-c2ccccc2)cc1 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)F ; FC(F)(F)C(F)(F)C(F)(F)C(F)(F)C(F)(F)C(F)(F)COc1nc(-c2ccc(-c3ccccc3)cc2)nc(N2CCN(c3nc(OCC(F)(F)C(F)(F)C(F)(F)C(F)(F)C(F)(F)C(F)(F)F)nc(-c4ccc(-c5ccccc5)cc4)n3)CC2)n1]

In a nitrogen atmosphere, one drop of 1,2-dibromoethane was added to a solution of magnesium (15.0 mmol) suspended in tetrahydrofuran (20 ml), and 4-bromobiphenyl (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z230) (yield: 39%).

The compound was identified by FDMS measurement.
FDMS: 1244

### Example 36′ Evaluation of Adhesiveness of Compound (Z230) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z230) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z230) was vapor-deposited, and a 2 mm×1 mm transparent region was formed.

### Example 37 Synthesis of Compound (Z231)

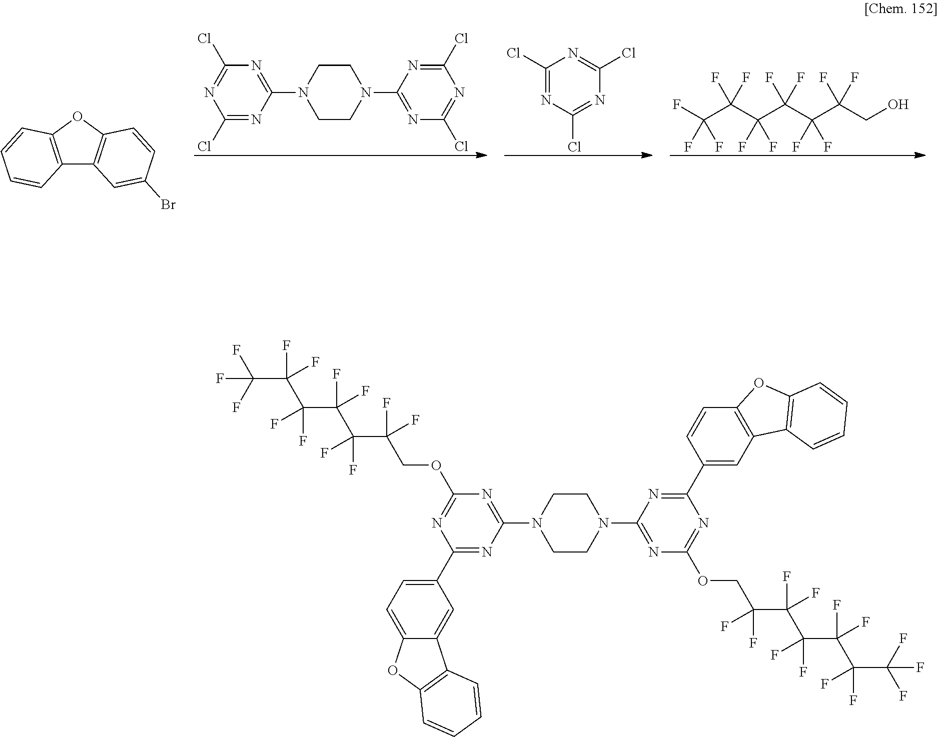 [SMILES: Brc1ccc2oc3ccccc3c2c1 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)F ; FC(F)(F)C(F)(F)C(F)(F)C(F)(F)C(F)(F)C(F)(F)COc1nc(-c2ccc3oc4ccccc4c3c2)nc(N2CCN(c3nc(OCC(F)(F)C(F)(F)C(F)(F)C(F)(F)C(F)(F)C(F)(F)F)nc(-c4ccc5oc6ccccc6c5c4)n3)CC2)n1]

In a nitrogen atmosphere, one drop of 1,2-dibromoethane was added to a solution of magnesium (15.0 mmol) suspended in tetrahydrofuran (20 ml), and 2-bromodibenzofuran (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z231) (yield: 22%).

The compound was identified by FDMS measurement.
FDMS: 1272

### Example 37′ Evaluation of Adhesiveness of Compound (Z231) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z231) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z231) was vapor-deposited, and a 2 mm×1 mm transparent region was formed.

### Example 38 Synthesis of Compound (Z232)

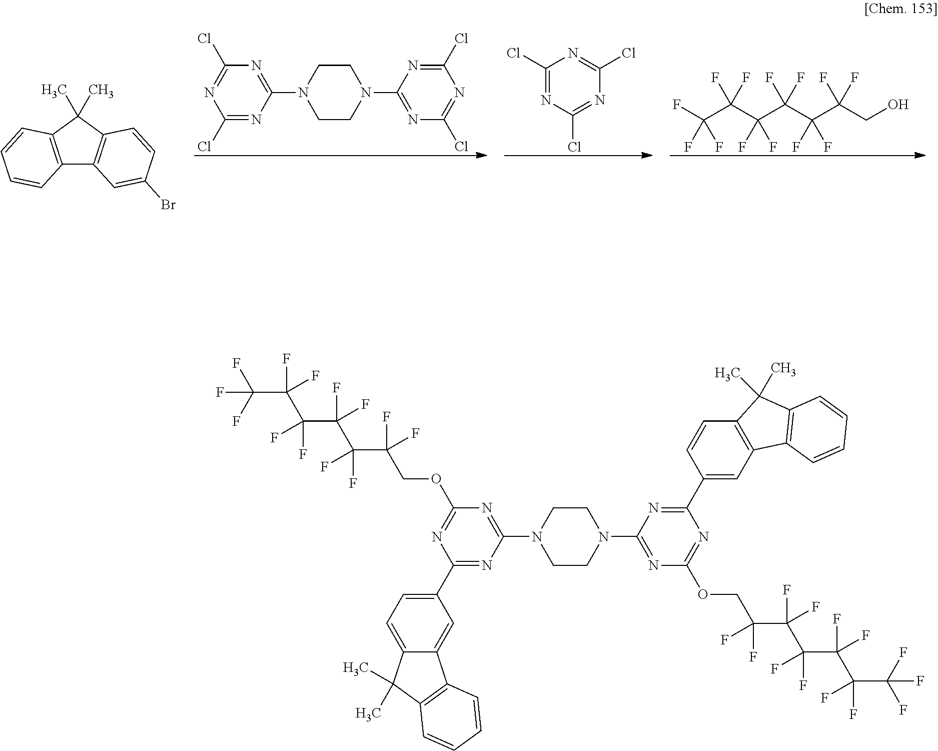 [SMILES: CC1(C)c2ccccc2-c2cc(Br)ccc21 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)F ; CC1(C)c2ccccc2-c2cc(-c3nc(OCC(F)(F)C(F)(F)C(F)(F)C(F)(F)C(F)(F)C(F)(F)F)nc(N4CCN(c5nc(OCC(F)(F)C(F)(F)C(F)(F)C(F)(F)C(F)(F)C(F)(F)F)nc(-c6ccc7c(c6)-c6ccccc6C7(C)C)n5)CC4)n3)ccc21]

In a nitrogen atmosphere, one drop of 1,2-dibromoethane was added to a solution of magnesium (15.0 mmol) suspended in tetrahydrofuran (20 ml), and 3-bromo-9,9-dimethyl-9H-fluorene (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z232) (yield: 15%).

The compound was identified by FDMS measurement.
FDMS: 1324

### Example 38′ Evaluation of Adhesiveness of Compound (Z232) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z232) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z232) was vapor-deposited, and a 2 mm×1 mm transparent region was formed.

### Example 39 Synthesis of Compound (Z233)

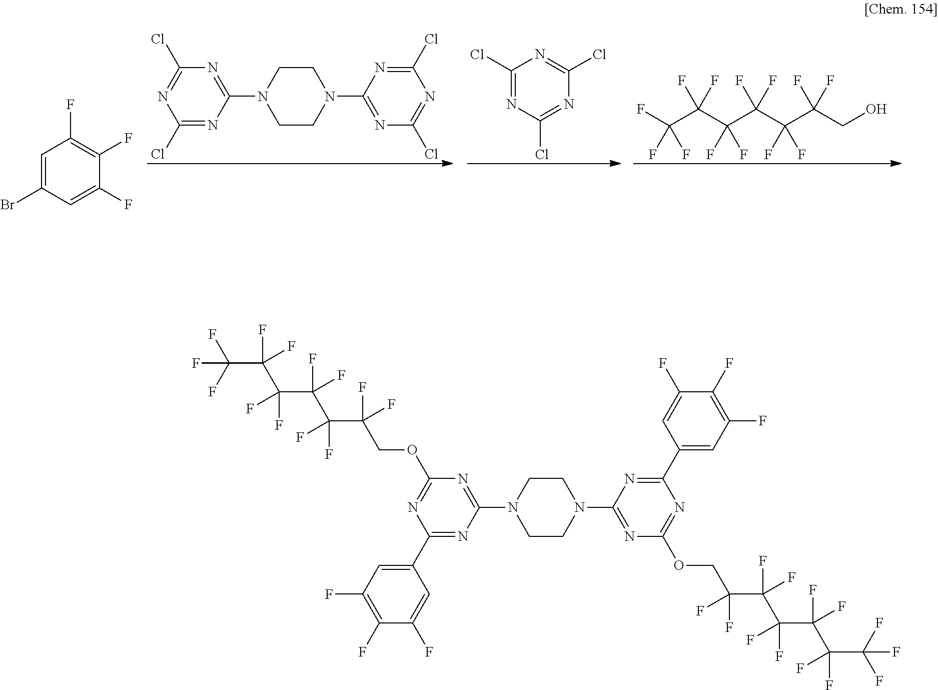

In a nitrogen atmosphere, one drop of 1,2-dibromoethane was added to a solution of magnesium (15.0 mmol) suspended in tetrahydrofuran (20 ml), and 1-bromo-3,4,5-trifluorophenylbenzene (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z233) (yield: 38%).

The compound was identified by FDMS measurement.
FDMS: 1200

### Example 39′ Evaluation of Adhesiveness of Compound (Z233) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z233) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z233) was vapor-deposited, and a 2 mm×1 mm transparent region was formed.

### Example 40 Synthesis of Compound (Z234)

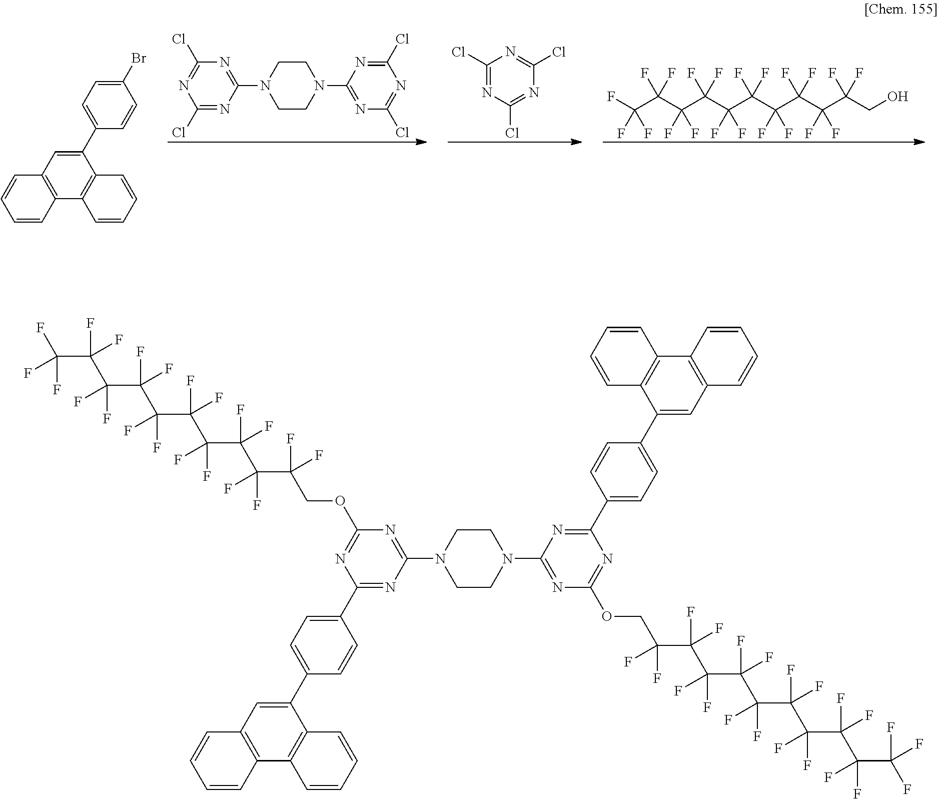 [SMILES: Brc1ccc(-c2cc3ccccc3c3ccccc23)cc1 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)C(F)(F)C(F)(F)C(F)(F)C(F)(F)F ; FC(F)(F)C(F)(F)C(F)(F)C(F)(F)C(F)(F)C(F)(F)C(F)(F)C(F)(F)C(F)(F)C(F)(F)COc1nc(-c2ccc(-c3cc4ccccc4c4ccccc34)cc2)nc(N2CCN(c3nc(OCC(F)(F)C(F)(F)C(F)(F)C(F)(F)C(F)(F)C(F)(F)C(F)(F)C(F)(F)C(F)(F)C(F)(F)F)nc(-c4ccc(-c5cc6ccccc6c6ccccc56)cc4)n3)CC2)n1]

In a nitrogen atmosphere, one drop of 1,2-dibromoethane was added to a solution of magnesium (15.0 mmol) suspended in tetrahydrofuran (20 ml), and 1-bromo-4-(9-phenanthryl)benzene (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-heneicosafluoro-1-undecanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z234) (yield: 41%).

The compound was identified by FDMS measurement.
FDMS: 1844

### Example 40′ Evaluation of Adhesiveness of Compound (Z234) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z234) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z234) was vapor-deposited, and a 2 mm×1 mm transparent region was formed.

### Example 41 Synthesis of Compound (Z235)

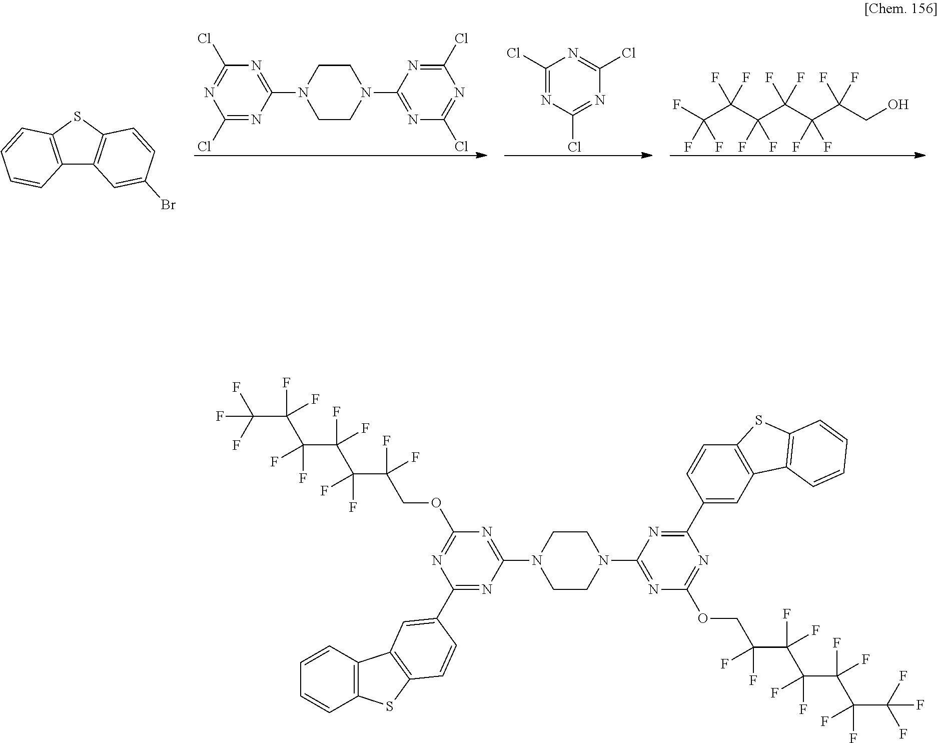 [SMILES: Brc1ccc2sc3ccccc3c2c1 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)F ; FC(F)(F)C(F)(F)C(F)(F)C(F)(F)C(F)(F)C(F)(F)COc1nc(-c2ccc3sc4ccccc4c3c2)nc(N2CCN(c3nc(OCC(F)(F)C(F)(F)C(F)(F)C(F)(F)C(F)(F)C(F)(F)F)nc(-c4ccc5sc6ccccc6c5c4)n3)CC2)n1]

In a nitrogen atmosphere, one drop of 1,2-dibromoethane was added to a solution of magnesium (15.0 mmol) suspended in tetrahydrofuran (20 ml), and 2-bromodibenzothiophene (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z235) (yield: 35%).

The compound was identified by FDMS measurement.
FDMS: 1304

### Example 41′ Evaluation of Adhesiveness of Compound (Z235) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z235) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z235) was vapor-deposited, and a 2 mm×1 mm transparent region was formed.

### Example 42 Synthesis of Compound (Z236)

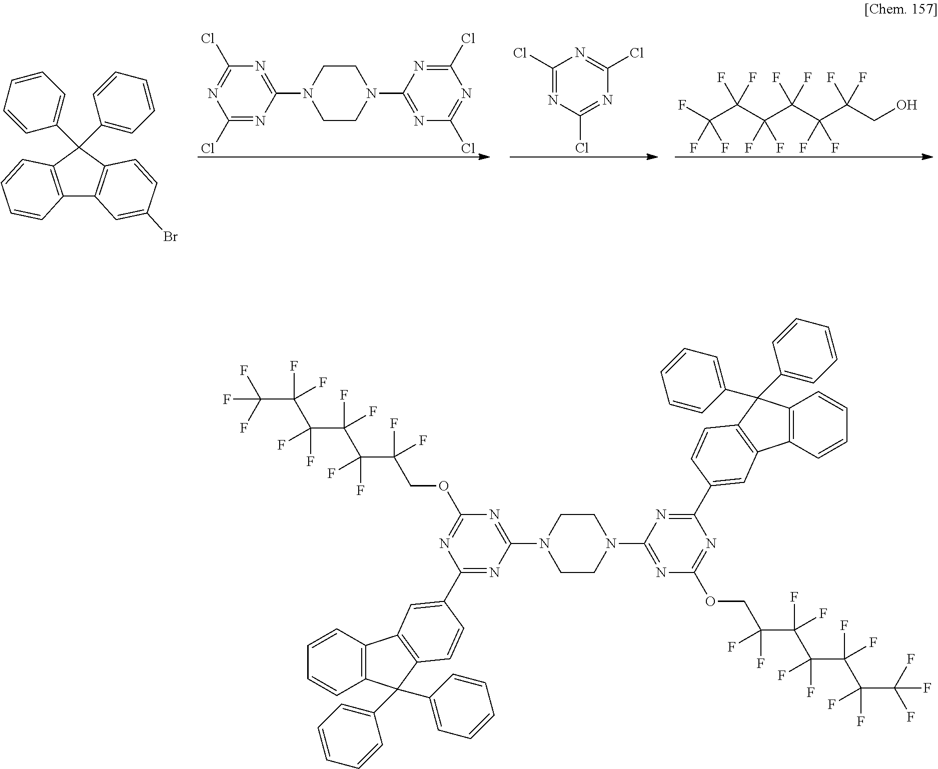 [SMILES: Brc1ccc2c(c1)-c1ccccc1C2(c1ccccc1)c1ccccc1 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)F ; FC(F)(F)C(F)(F)C(F)(F)C(F)(F)C(F)(F)C(F)(F)COc1nc(-c2ccc3c(c2)-c2ccccc2C3(c2ccccc2)c2ccccc2)nc(N2CCN(c3nc(OCC(F)(F)C(F)(F)C(F)(F)C(F)(F)C(F)(F)C(F)(F)F)nc(-c4ccc5c(c4)-c4ccccc4C5(c4ccccc4)c4ccccc4)n3)CC2)n1]

In a nitrogen atmosphere, one drop of 1,2-dibromoethane was added to a solution of magnesium (15.0 mmol) suspended in tetrahydrofuran (20 ml), and 3-bromo-9,9-diphenyl-9H-fluorene (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z236) (yield: 29%).

The compound was identified by FDMS measurement.
FDMS: 1572

### Example 42′ Evaluation of Adhesiveness of Compound (Z236) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z236) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z236) was vapor-deposited, and a 2 mm×1 mm transparent region was formed.

### Example 43 Synthesis of Compound (Z237)

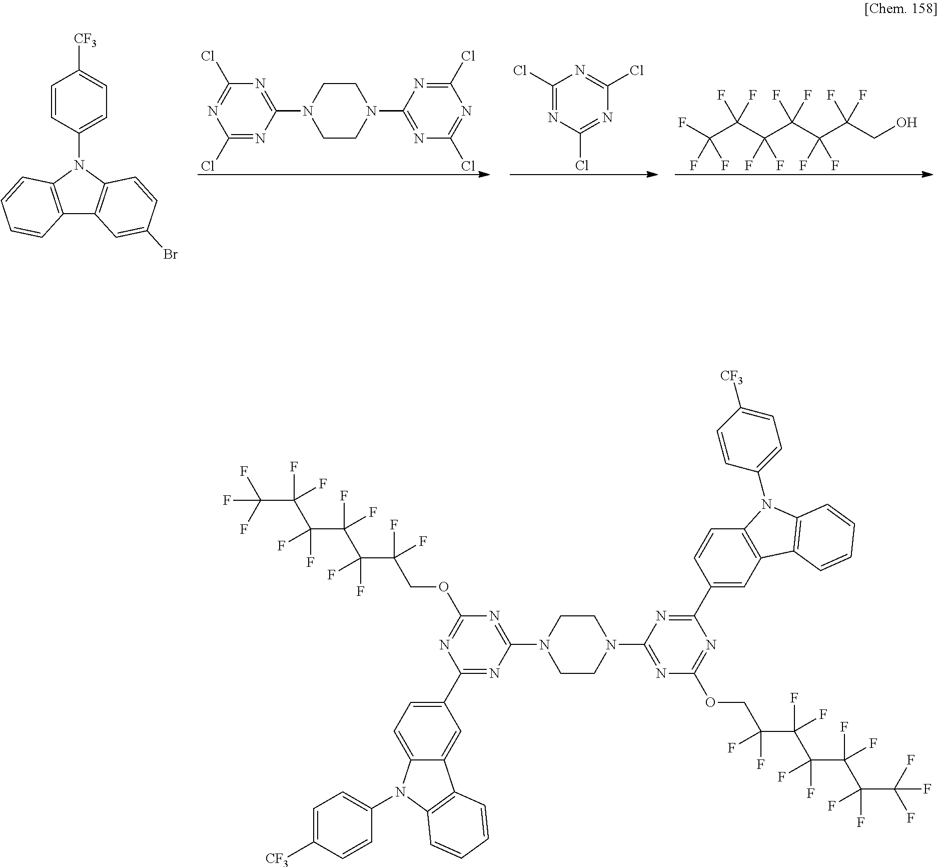 [SMILES: FC(F)(F)c1ccc(-n2c3ccccc3c3cc(Br)ccc32)cc1 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)C(F)(F)C(F)(F)C(F)(F)F ; FC(F)(F)c1ccc(-n2c3ccccc3c3cc(-c4nc(OCC(F)(F)C(F)(F)C(F)(F)C(F)(F)C(F)(F)C(F)(F)F)nc(N5CCN(c6nc(OCC(F)(F)C(F)(F)C(F)(F)C(F)(F)C(F)(F)C(F)(F)F)nc(-c7ccc8c(c7)c7ccccc7n8-c7ccc(C(F)(F)F)cc7)n6)CC5)n4)ccc32)cc1]

In a nitrogen atmosphere, one drop of 1,2-dibromoethane was added to a solution of magnesium (15.0 mmol) suspended in tetrahydrofuran (20 ml), and 3-bromo-9-(4-trifluoromethylphenyl) carbazole (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z237) (yield: 29%).

The compound was identified by FDMS measurement.
FDMS: 1558

### Example 43′ Evaluation of Adhesiveness of Compound (Z237) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z237) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z237) was vapor-deposited, and a 2 mm×1 mm transparent region was formed.

### Example 44 Synthesis of Compound (Z238)

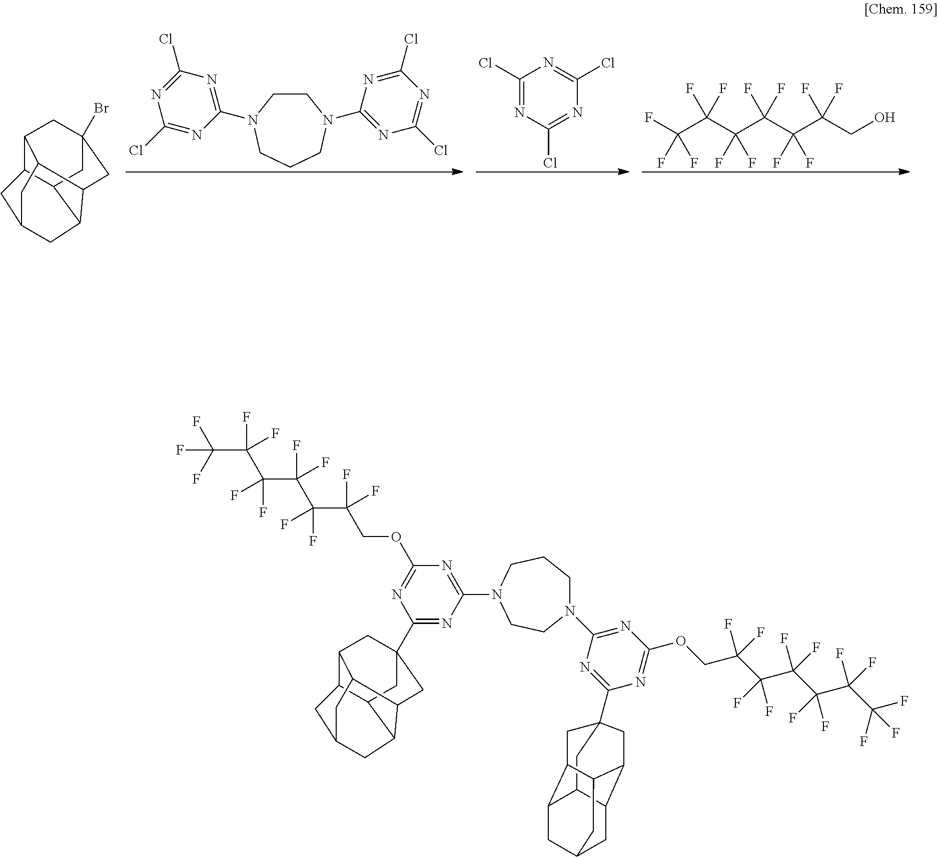

In a nitrogen atmosphere, one drop of 1,2-dibromoethane was added to a solution of magnesium (15.0 mmol) suspended in tetrahydrofuran (20 ml), and 4-bromodiamantane (14.0 mmol) dissolved in tetrahydrofuran (20 ml) was added dropwise over 20 minutes. The dropwise addition was followed by stirring at 60° C. for 3 hours. The temperature was then returned to room temperature, and S2 (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z238) (yield: 58%).

The compound was identified by FDMS measurement.
FDMS: 1326

### Example 44′ Evaluation of Adhesiveness of Compound (Z238) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z238) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z238) was vapor-deposited, and a 2 mm×1 mm transparent region was formed.

### Example 45 Synthesis of Compound (Z239)

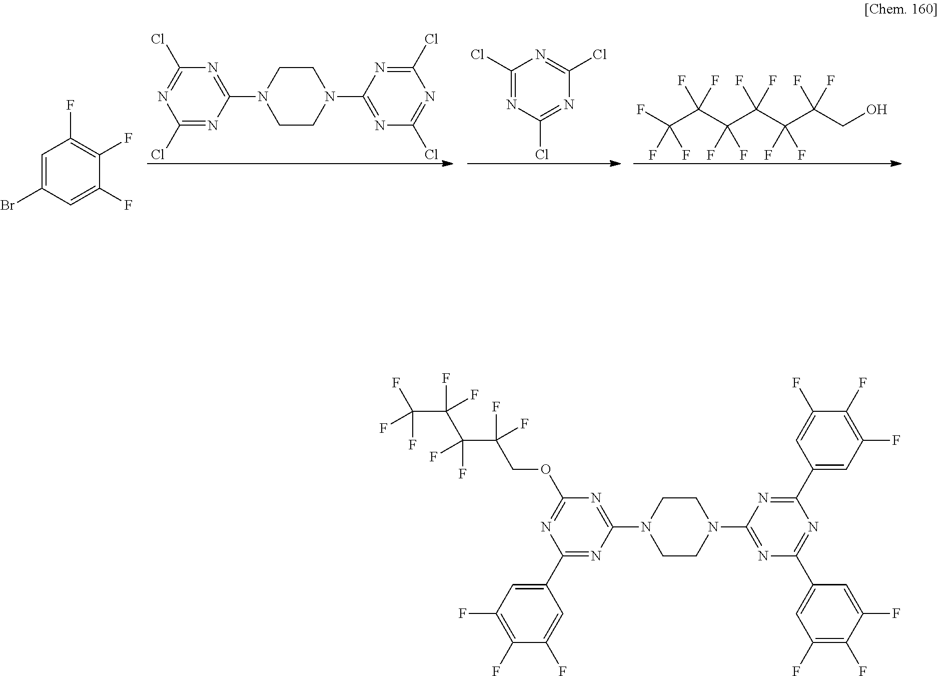

In a nitrogen atmosphere, one drop of 1,2-dibromoethane was added to a solution of magnesium (22.0 mmol) suspended in tetrahydrofuran (20 ml), and 1-bromo-3,4,5-trifluorophenylbenzene (21.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (8.0 mmol) was suspended in tetrahydrofuran (20 ml) and was stirred at 0° C., and 1H, 1H-nonafluoro-1-pentanol (7.5 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z239) (yield: 11%).

The compound was identified by FDMS measurement.
FDMS: 882

### Example 45′ Evaluation of Adhesiveness of Compound (Z239) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z239) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z239) was vapor-deposited, and a 2 mm×1 mm transparent region was formed.

### Example 46 Synthesis of Compound (Z240)

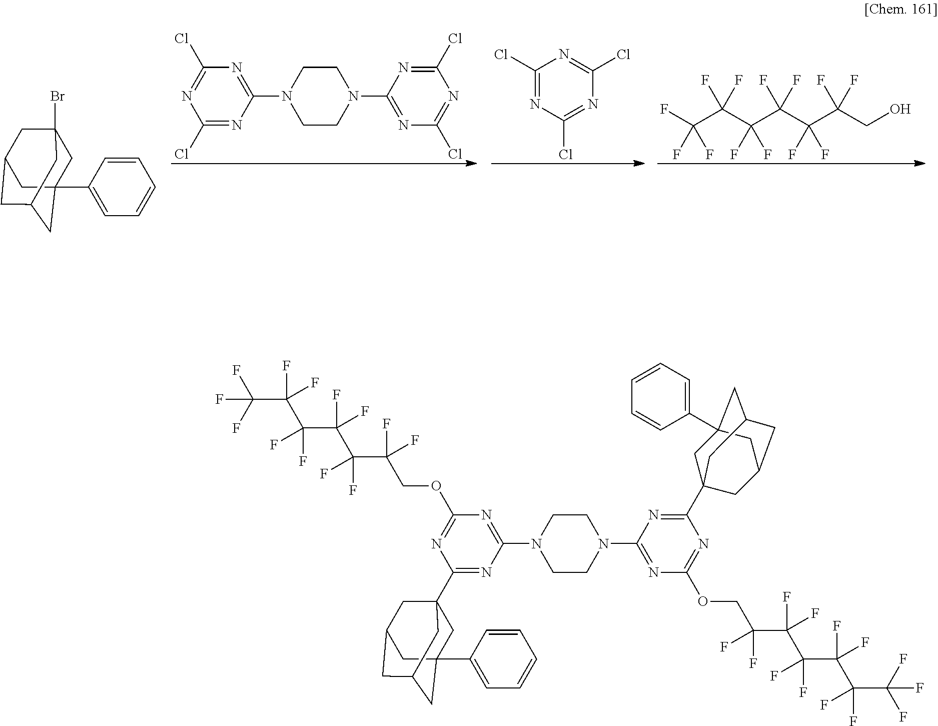

In a nitrogen atmosphere, one drop of 1,2-dibromoethane was added to a solution of magnesium (15.0 mmol) suspended in tetrahydrofuran (20 ml), and 1-bromo-3-phenyladamantane (14.0 mmol) dissolved in tetrahydrofuran (20 ml) was added dropwise over 20 minutes. The dropwise addition was followed by stirring at 60° C. for 3 hours. The temperature was then returned to room temperature, and 1,4-bis(4,6-dichloro-1,3,5-triazin-2-yl) piperidine (7.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z240) (yield: 49%).

The compound was identified by FDMS measurement.
FDMS: 1360

### Example 46′ Evaluation of Adhesiveness of Compound (Z240) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z240) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z240) was vapor-deposited, and a 2 mm×1 mm transparent region was formed.

### Example 47 Synthesis of Compound (Z241)

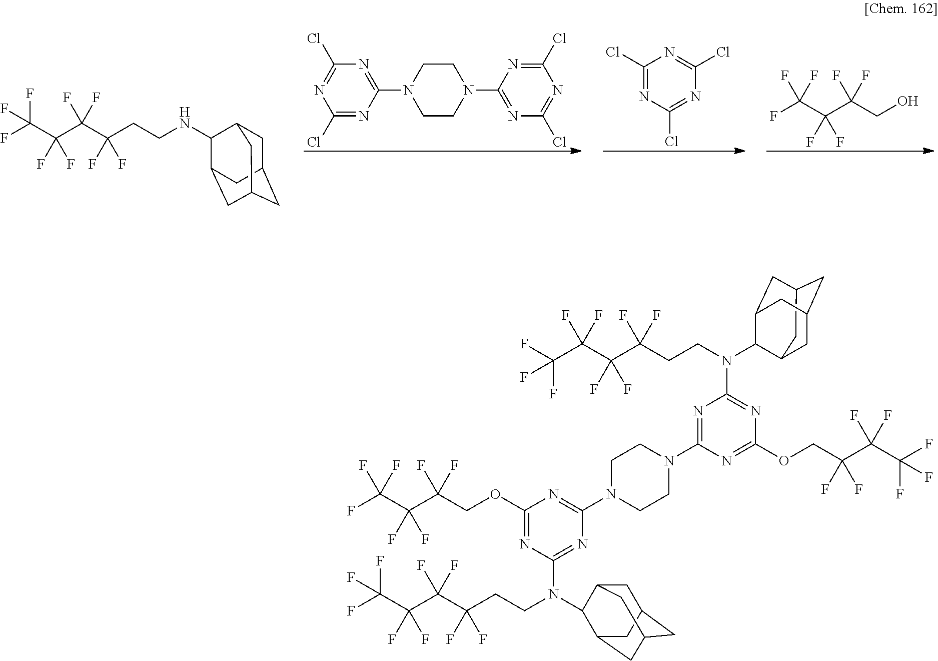 [SMILES: FC(F)(F)C(F)(F)C(F)(F)C(F)(F)CCNC1C2CC3CC(C2)CC1C3 ; Clc1nc(Cl)nc(N2CCN(c3nc(Cl)nc(Cl)n3)CC2)n1 ; Clc1nc(Cl)nc(Cl)n1 ; OCC(F)(F)C(F)(F)C(F)(F)F ; FC(F)(F)C(F)(F)C(F)(F)COc1nc(N2CCN(c3nc(OCC(F)(F)C(F)(F)C(F)(F)F)nc(N(CCC(F)(F)C(F)(F)C(F)(F)C(F)(F)F)C4C5CC6CC(C5)CC4C6)n3)CC2)nc(N(CCC(F)(F)C(F)(F)C(F)(F)C(F)(F)F)C2C3CC4CC(C3)CC2C4)n1]

In a nitrogen atmosphere, sodium hydride (16.0 mmol) was suspended in tetrahydrofuran (30 ml) and was stirred at 0° C., and 1-(1H, 1H, 2H, 2H-nonafluorohexyl-1-amino) adamantane (14.0 mmol) was added dropwise to the suspension over 20 minutes. After completion of the dropwise addition, the suspension was returned to room temperature and was stirred for 2 hours. 1,4-bis(4,6-dichloro-1,3,5-triazin-2-yl) piperidine (7.0 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-heptafluoro-1-butanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z241) (yield: 25%).

The compound was identified by FDMS measurement.
FDMS: 1430

### Example 47′ Evaluation of Adhesiveness of Compound (Z241) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z241) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z241) was vapor-deposited, and a 2 mm×1 mm transparent region was formed.

### Example 48 Synthesis of Compound (Z242)

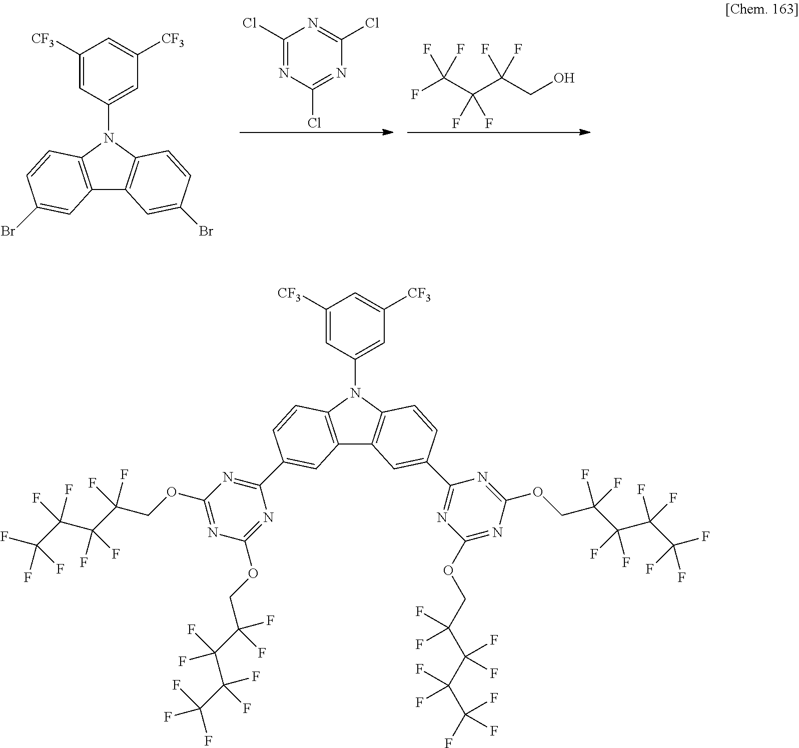 [SMILES: FC(F)(F)c1cc(-n2c3ccc(Br)cc3c3cc(Br)ccc32)cc(C(F)(F)F)c1 ; Clc1nc(Cl)nc(Cl)n1 ; OCC(F)(F)C(F)(F)C(F)(F)F ; FC(F)(F)c1cc(-n2c3ccc(-c4nc(OCC(F)(F)C(F)(F)C(F)(F)C(F)(F)F)nc(OCC(F)(F)C(F)(F)C(F)(F)C(F)(F)F)n4)cc3c3cc(-c4nc(OCC(F)(F)C(F)(F)C(F)(F)C(F)(F)F)nc(OCC(F)(F)C(F)(F)C(F)(F)C(F)(F)F)n4)ccc32)cc(C(F)(F)F)c1]

In a nitrogen atmosphere, one drop of 1,2-dibromoethane was added to a solution of magnesium (22.0 mmol) suspended in tetrahydrofuran (20 ml), and 3,6-dibromo-9-(3,5-ditrifluoromethylphenyl) carbazole (10.0 mmol) dissolved in tetrahydrofuran (20 ml) was added dropwise over 20 minutes. The dropwise addition was followed by stirring at 60° C. for 3 hours. The temperature was then returned to room temperature, and cyanuric chloride (22.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (50.0 mmol) was suspended in tetrahydrofuran (20 ml) and was stirred at 0° C., and 1H, 1H-nonafluoro-1-pentanol (45.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z242) (yield: 18%).

The compound was identified by FDMS measurement.
FDMS: 1529

### Example 48′ Evaluation of Adhesiveness of Compound (Z242) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z242) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z242) was vapor-deposited, and a 2 mm×1 mm transparent region was formed.

### Example 49 Synthesis of Compound (Z243)

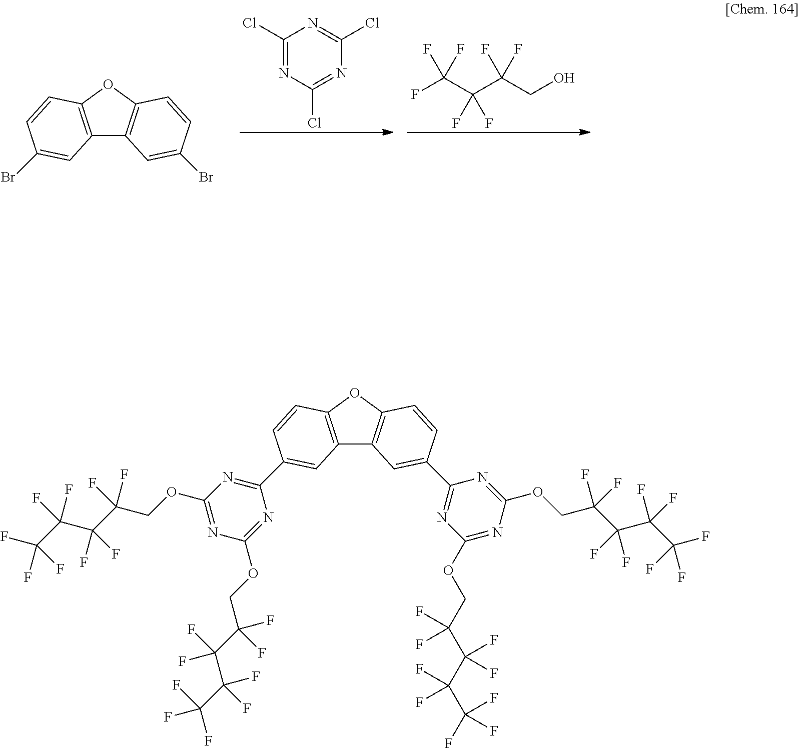 [SMILES: Brc1ccc2oc3ccc(Br)cc3c2c1 ; Clc1nc(Cl)nc(Cl)n1 ; OCC(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(-c2ccc3oc4ccc(-c5nc(OCC(F)(F)C(F)(F)C(F)(F)C(F)(F)F)nc(OCC(F)(F)C(F)(F)C(F)(F)C(F)(F)F)n5)cc4c3c2)n1]

In a nitrogen atmosphere, one drop of 1,2-dibromoethane was added to a solution of magnesium (22.0 mmol) suspended in tetrahydrofuran (20 ml), and 2,8-dibromodibenzofuran (10.0 mmol) dissolved in tetrahydrofuran (20 ml) was added dropwise over 20 minutes. The dropwise addition was followed by stirring at 60° C. for 3 hours. The temperature was then returned to room temperature, and cyanuric chloride (22.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (50.0 mmol) was suspended in tetrahydrofuran (20 ml) and was stirred at 0° C., and 1H, 1H-nonafluoro-1-pentanol (45.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z243) (yield: 22%).

The compound was identified by FDMS measurement.
FDMS: 1318

### Example 49′ Evaluation of Adhesiveness of Compound (Z243) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z243) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z243) was vapor-deposited, and a 2 mm×1 mm transparent region was formed.

### Example 50 Synthesis of Compound (Z244)

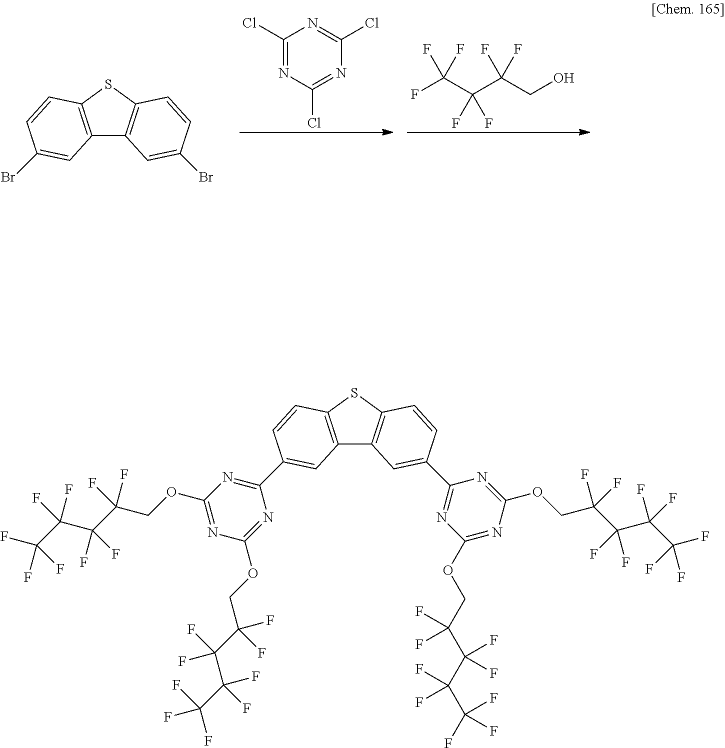 [SMILES: Brc1ccc2sc3ccc(Br)cc3c2c1 ; Clc1nc(Cl)nc(Cl)n1 ; OCC(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(-c2ccc3sc4ccc(-c5nc(OCC(F)(F)C(F)(F)C(F)(F)C(F)(F)F)nc(OCC(F)(F)C(F)(F)C(F)(F)C(F)(F)F)n5)cc4c3c2)n1]

In a nitrogen atmosphere, one drop of 1,2-dibromoethane was added to a solution of magnesium (22.0 mmol) suspended in tetrahydrofuran (20 ml), and 2,8-dibromodibenzothiophene (10.0 mmol) dissolved in tetrahydrofuran (20 ml) was added dropwise over 20 minutes. The dropwise addition was followed by stirring at 60° C. for 3 hours. The temperature was then returned to room temperature, and cyanuric chloride (22.0 mmol) dissolved in tetrahydrofuran (30 ml) was added dropwise over 20 minutes. The dropwise addition was followed by stirring at 60° C. for 3 hours. Next, in the reactor cooled to 0° C., in a nitrogen atmosphere, sodium hydride (50.0 mmol) was suspended in tetrahydrofuran (20 ml) and was stirred at 0° C., and 1H, 1H-nonafluoro-1-pentanol (45.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z244) (yield: 25%).

The compound was identified by FDMS measurement.
FDMS: 1334

### Example 50′ Evaluation of Adhesiveness of Compound (Z244) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z244) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z244) was vapor-deposited, and a 2 mm×1 mm transparent region was formed.

### Example 51 Synthesis of Compound (Z245)

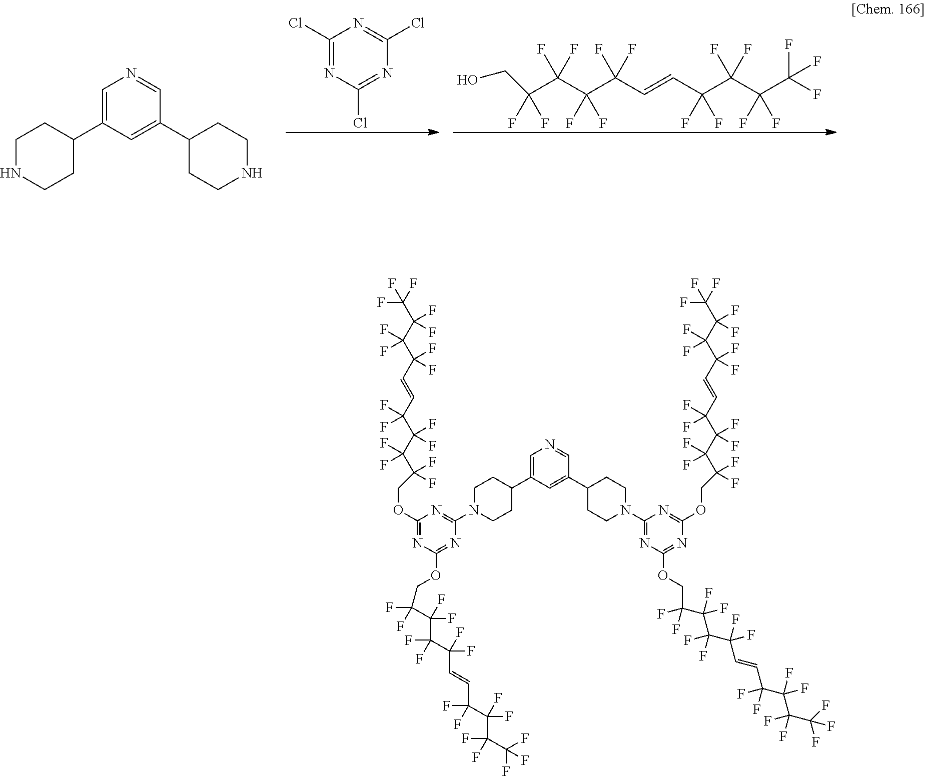 [SMILES: c1ncc(C2CCNCC2)cc1C1CCNCC1 ; Clc1nc(Cl)nc(Cl)n1 ; OCC(F)(F)C(F)(F)C(F)(F)C(F)(F)/C=C/C(F)(F)C(F)(F)C(F)(F)C(F)(F)F ; FC(F)(F)C(F)(F)C(F)(F)C(F)(F)/C=C/C(F)(F)C(F)(F)C(F)(F)C(F)(F)COc1nc(OCC(F)(F)C(F)(F)C(F)(F)C(F)(F)/C=C/C(F)(F)C(F)(F)C(F)(F)C(F)(F)F)nc(N2CCC(c3cncc(C4CCN(c5nc(OCC(F)(F)C(F)(F)C(F)(F)C(F)(F)/C=C/C(F)(F)C(F)(F)C(F)(F)C(F)(F)F)nc(OCC(F)(F)C(F)(F)C(F)(F)C(F)(F)/C=C/C(F)(F)C(F)(F)C(F)(F)C(F)(F)F)n5)CC4)c3)CC2)n1]

In a nitrogen atmosphere, sodium hydride (15.0 mmol) was suspended in tetrahydrofuran (30 ml) and was stirred at 0° C., and 3,5-di(4-piperidyl)pyridine (7.0 mmol) was added dropwise to the suspension over 20 minutes. After completion of the dropwise addition, the suspension was returned to room temperature and was stirred for 2 hours. Cyanuric chloride (15.0 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (32.0 mmol) was suspended in tetrahydrofuran (20 ml) and was stirred at 0° C., and 2, 2, 3, 3, 4, 4, 5, 5, 8, 8, 9, 9, 10, 10, 11, 11, 11-heptadecafluoro-6-undecen-1-ol (30.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z245) (yield: 18%).

The compound was identified by FDMS measurement.
FDMS: 2299

### Example 51′ Evaluation of Adhesiveness of Compound (Z245) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z245) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z245) was vapor-deposited, and a 2 mm×1 mm transparent region was formed.

### Example 52 Synthesis of Compound (Z246)

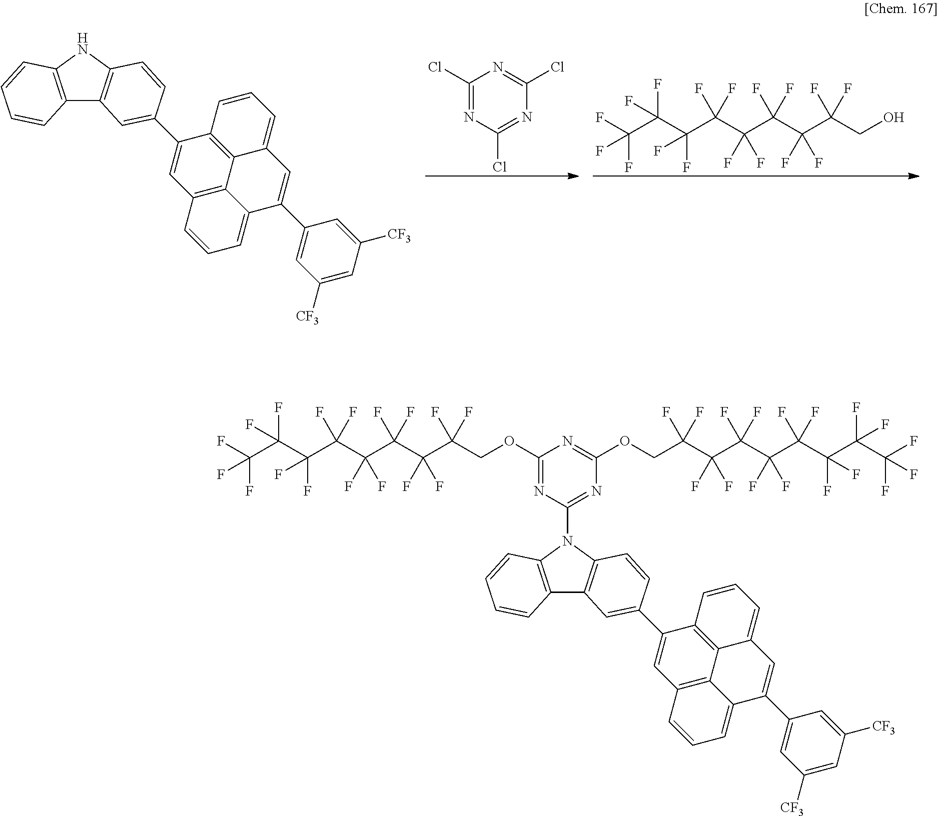 [SMILES: FC(F)(F)c1cc(-c2cc3cccc4c(-c5ccc6[nH]c7ccccc7c6c5)cc5cccc2c5c34)cc(C(F)(F)F)c1 ; Clc1nc(Cl)nc(Cl)n1 ; OCC(F)(F)C(F)(F)C(F)(F)C(F)(F)C(F)(F)C(F)(F)C(F)(F)C(F)(F)F ; FC(F)(F)c1cc(-c2cc3cccc4c(-c5ccc6c(c5)c5ccccc5n6-c5nc(OCC(F)(F)C(F)(F)C(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)C(F)(F)C(F)(F)F)n5)cc5cccc2c5c34)cc(C(F)(F)F)c1]

In a nitrogen atmosphere, sodium hydride (7.5 mmol) was suspended in tetrahydrofuran (30 ml) and was stirred at 0° C., and 3-[9-(3,5-ditrifluoromethylphenyl)-4-pyrenyl]-9H-carbazole (7.0 mmol) was added dropwise to the suspension over 20 minutes. After completion of the dropwise addition, the suspension was returned to room temperature and was stirred for 2 hours. Cyanuric chloride (7.5 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-heptadecafluoro-1-nonanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z246) (yield: 21%).

The compound was identified by FDMS measurement.
FDMS: 1554

### Example 52′ Evaluation of Adhesiveness of Compound (Z246) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z246) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z246) was vapor-deposited, and a 2 mm×1 mm transparent region was formed.

### Example 53 Synthesis of Compound (Z247)

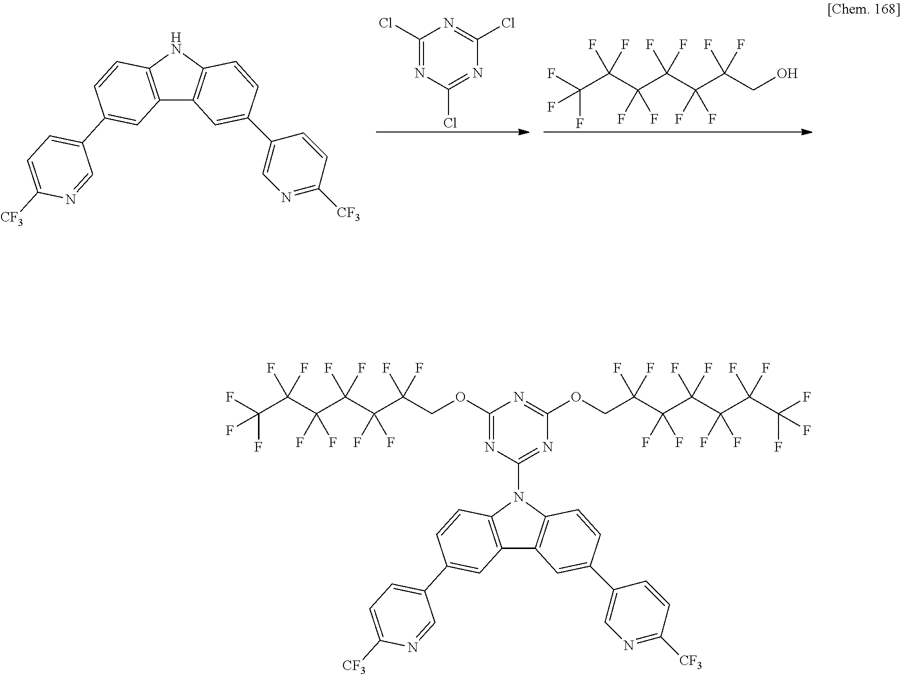 [SMILES: FC(F)(F)c1ccc(-c2ccc3[nH]c4ccc(-c5ccc(C(F)(F)F)nc5)cc4c3c2)cn1 ; Clc1nc(Cl)nc(Cl)n1 ; OCC(F)(F)C(F)(F)C(F)(F)C(F)(F)C(F)(F)C(F)(F)F ; FC(F)(F)c1ccc(-c2ccc3c(c2)c2cc(-c4ccc(C(F)(F)F)nc4)ccc2n3-c2nc(OCC(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)F)n2)cn1]

In a nitrogen atmosphere, sodium hydride (7.5 mmol) was suspended in tetrahydrofuran (30 ml) and was stirred at 0° C., and 3-[9-(3,5-ditrifluoromethylphenyl)-4-pyrenyl]-9H-carbazole (7.0 mmol) was added dropwise to the suspension over 20 minutes. After completion of the dropwise addition, the suspension was returned to room temperature and was stirred for 2 hours. Cyanuric chloride (7.5 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-tridecafluoro-1-heptanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z247) (yield: 22%).

The compound was identified by FDMS measurement.
FDMS: 1232

### Example 53′ Evaluation of Adhesiveness of Compound (Z247) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z247) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z247) was vapor-deposited, and a 2 mm×1 mm transparent region was formed.

### Example 54 Synthesis of Compound (Z248)

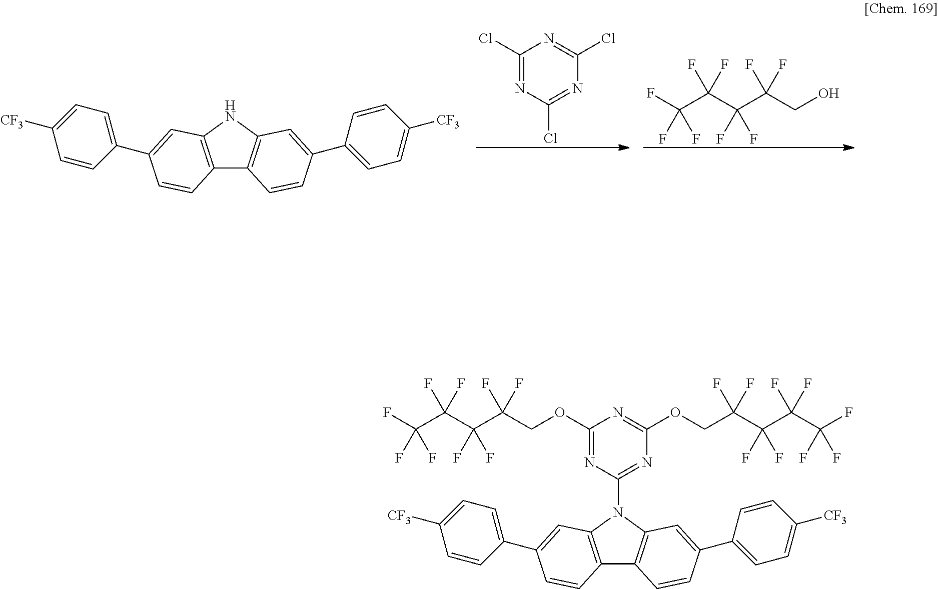 [SMILES: FC(F)(F)c1ccc(-c2ccc3c(c2)[nH]c2cc(-c4ccc(C(F)(F)F)cc4)ccc23)cc1 ; Clc1nc(Cl)nc(Cl)n1 ; OCC(F)(F)C(F)(F)C(F)(F)C(F)(F)F ; FC(F)(F)c1ccc(-c2ccc3c4ccc(-c5ccc(C(F)(F)F)cc5)cc4n(-c4nc(OCC(F)(F)C(F)(F)C(F)(F)C(F)(F)F)nc(OCC(F)(F)C(F)(F)C(F)(F)C(F)(F)F)n4)c3c2)cc1]

In a nitrogen atmosphere, sodium hydride (7.5 mmol) was suspended in tetrahydrofuran (30 ml) and was stirred at 0° C., and 2,7-di(4-trifluoromethylphenyl)-9H-carbazole (7.0 mmol) was added dropwise to the suspension over 20 minutes. After completion of the dropwise addition, the suspension was returned to room temperature and was stirred for 2 hours. Cyanuric chloride (7.5 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (16.0 mmol) was suspended in tetrahydrofuran (20 ml) and was stirred at 0° C., and 1H, 1H-nonafluoro-1-pentanol (15.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z248) (yield: 23%).

The compound was identified by FDMS measurement.
FDMS: 1030

### Example 54′ Evaluation of Adhesiveness of Compound (Z248) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z248) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z248) was vapor-deposited, and a 2 mm×1 mm transparent region was formed.

### Example 55 Synthesis of Compound (Z249)

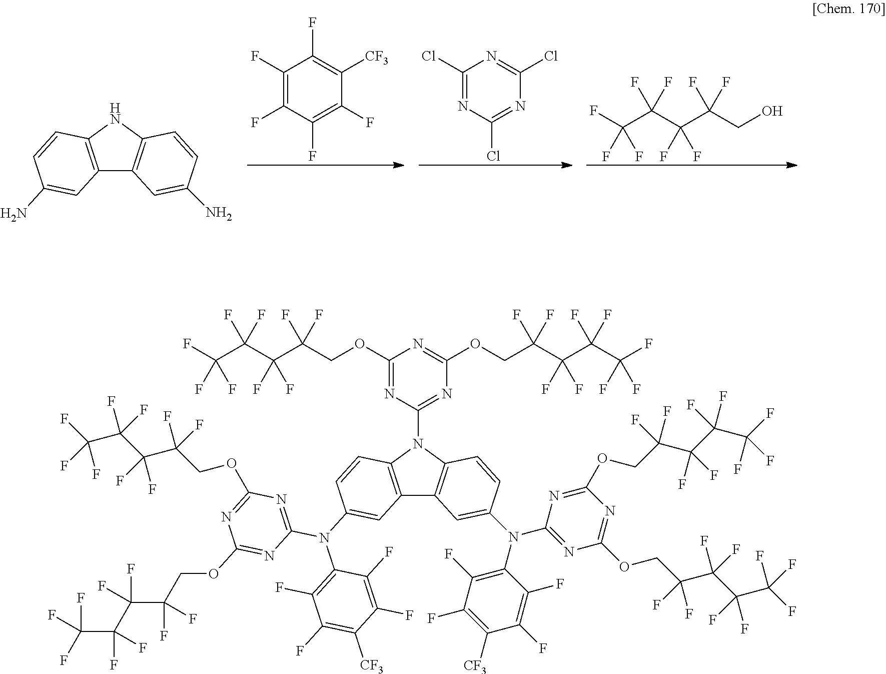 [SMILES: Nc1ccc2[nH]c3ccc(N)cc3c2c1 ; Fc1c(F)c(F)c(C(F)(F)F)c(F)c1F ; Clc1nc(Cl)nc(Cl)n1 ; OCC(F)(F)C(F)(F)C(F)(F)C(F)(F)F ; Fc1c(F)c(C(F)(F)F)c(F)c(F)c1N(c1ccc2c(c1)c1cc(N(c3nc(OCC(F)(F)C(F)(F)C(F)(F)C(F)(F)F)nc(OCC(F)(F)C(F)(F)C(F)(F)C(F)(F)F)n3)c3c(F)c(F)c(C(F)(F)F)c(F)c3F)ccc1n2-c1nc(OCC(F)(F)C(F)(F)C(F)(F)C(F)(F)F)nc(OCC(F)(F)C(F)(F)C(F)(F)C(F)(F)F)n1)c1nc(OCC(F)(F)C(F)(F)C(F)(F)C(F)(F)F)nc(OCC(F)(F)C(F)(F)C(F)(F)C(F)(F)F)n1]

In a nitrogen atmosphere, 3,6-diamino-9H-carbazole (10.0 mmol), perfluorotoluene (30.0 mmol) and tripotassium phosphate (50 mmol) were suspended in dimethyl sulfoxide (100 mL) and were stirred at 80° C. for 6 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S21. Subsequently, in a nitrogen atmosphere, sodium hydride (21.5 mmol) was suspended in tetrahydrofuran (30 ml) and was stirred at 0° C., and S21 (7.0 mmol) was added dropwise to the suspension over 20 minutes. After completion of the dropwise addition, the suspension was returned to room temperature and was stirred for 2 hours. Cyanuric chloride (22 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (48.0 mmol) was suspended in tetrahydrofuran (20 ml) and was stirred at 0° C., and 1H, 1H-nonafluoro-1-pentanol (45.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z249) (yield: 25%).

The compound was identified by FDMS measurement.
FDMS: 2354

### Example 55′ Evaluation of Adhesiveness of Compound (Z249) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z249) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z249) was vapor-deposited, and a 2 mm×1 mm transparent region was formed.

### Example 56 Synthesis of Compound (Z250)

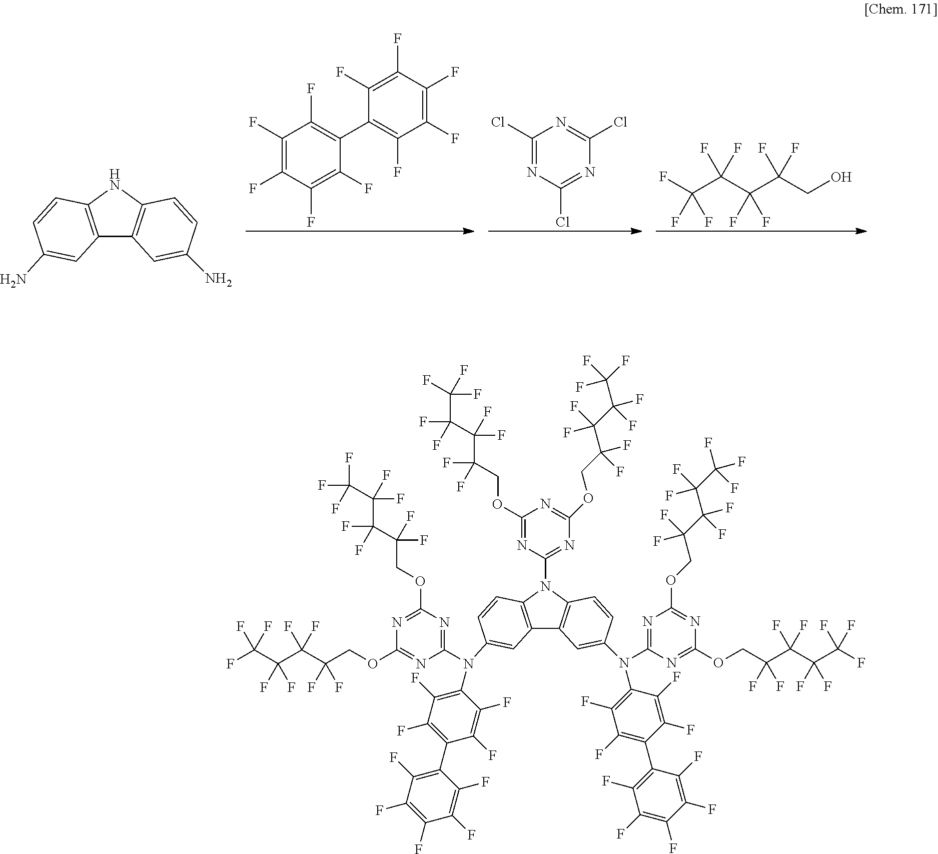 [SMILES: Nc1ccc2[nH]c3ccc(N)cc3c2c1 ; Fc1c(F)c(F)c(-c2c(F)c(F)c(F)c(F)c2F)c(F)c1F ; Clc1nc(Cl)nc(Cl)n1 ; OCC(F)(F)C(F)(F)C(F)(F)C(F)(F)F ; Fc1c(F)c(F)c(-c2c(F)c(F)c(N(c3ccc4c(c3)c3cc(N(c5nc(OCC(F)(F)C(F)(F)C(F)(F)C(F)(F)F)nc(OCC(F)(F)C(F)(F)C(F)(F)C(F)(F)F)n5)c5c(F)c(F)c(-c6c(F)c(F)c(F)c(F)c6F)c(F)c5F)ccc3n4-c3nc(OCC(F)(F)C(F)(F)C(F)(F)C(F)(F)F)nc(OCC(F)(F)C(F)(F)C(F)(F)C(F)(F)F)n3)c3nc(OCC(F)(F)C(F)(F)C(F)(F)C(F)(F)F)nc(OCC(F)(F)C(F)(F)C(F)(F)C(F)(F)F)n3)c(F)c2F)c(F)c1F]

In a nitrogen atmosphere, 3,6-diamino-9H-carbazole (10.0 mmol), perfluorobiphenyl (30.0 mmol) and tripotassium phosphate (50 mmol) were suspended in dimethyl sulfoxide (100 mL) and were stirred at 80° C. for 6 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S22. Subsequently, in a nitrogen atmosphere, sodium hydride (21.5 mmol) was suspended in tetrahydrofuran (30 ml) and was stirred at 0° C., and S22 (7.0 mmol) was added dropwise to the suspension over 20 minutes. After completion of the dropwise addition, the suspension was returned to room temperature and was stirred for 2 hours. Cyanuric chloride (22 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (48.0 mmol) was suspended in tetrahydrofuran (20 ml) and was stirred at 0° C., and 1H, 1H-nonafluoro-1-pentanol (45.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z250) (yield: 29%).

The compound was identified by FDMS measurement.
FDMS: 2550

### Example 56′ Evaluation of Adhesiveness of Compound (Z250) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z250) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z250) was vapor-deposited, and a 2 mm×1 mm transparent region was formed.

### Example 57 Synthesis of Compound (Z251)

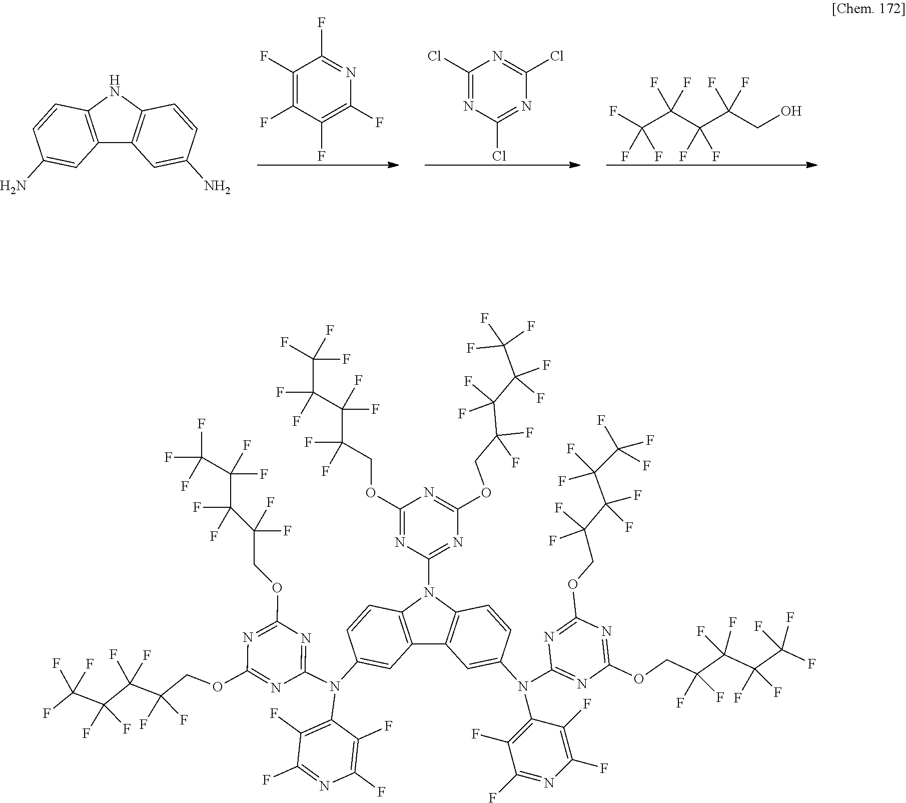 [SMILES: Nc1ccc2[nH]c3ccc(N)cc3c2c1 ; Fc1nc(F)c(F)c(F)c1F ; Clc1nc(Cl)nc(Cl)n1 ; OCC(F)(F)C(F)(F)C(F)(F)C(F)(F)F ; Fc1nc(F)c(F)c(N(c2ccc3c(c2)c2cc(N(c4nc(OCC(F)(F)C(F)(F)C(F)(F)C(F)(F)F)nc(OCC(F)(F)C(F)(F)C(F)(F)C(F)(F)F)n4)c4c(F)c(F)nc(F)c4F)ccc2n3-c2nc(OCC(F)(F)C(F)(F)C(F)(F)C(F)(F)F)nc(OCC(F)(F)C(F)(F)C(F)(F)C(F)(F)F)n2)c2nc(OCC(F)(F)C(F)(F)C(F)(F)C(F)(F)F)nc(OCC(F)(F)C(F)(F)C(F)(F)C(F)(F)F)n2)c1F]

In a nitrogen atmosphere, 3,6-diamino-9H-carbazole (10.0 mmol), perfluoropyridine (30.0 mmol) and tripotassium phosphate (50 mmol) were suspended in dimethyl sulfoxide (100 mL) and were stirred at 80° C. for 6 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S23. Subsequently, in a nitrogen atmosphere, sodium hydride (21.5 mmol) was suspended in tetrahydrofuran (30 ml) and was stirred at 0° C., and S23 (7.0 mmol) was added dropwise to the suspension over 20 minutes. After completion of the dropwise addition, the suspension was returned to room temperature and was stirred for 2 hours. Cyanuric chloride (22 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (48.0 mmol) was suspended in tetrahydrofuran (20 ml) and was stirred at 0° C., and 1H, 1H-nonafluoro-1-pentanol (45.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z251) (yield: 35%).

The compound was identified by FDMS measurement.
FDMS: 2550

### Example 57′ Evaluation of Adhesiveness of Compound (Z251) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z251) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z251) was vapor-deposited, and a 2 mm×1 mm transparent region was formed.

### Example 58 Synthesis of Compound (Z252)

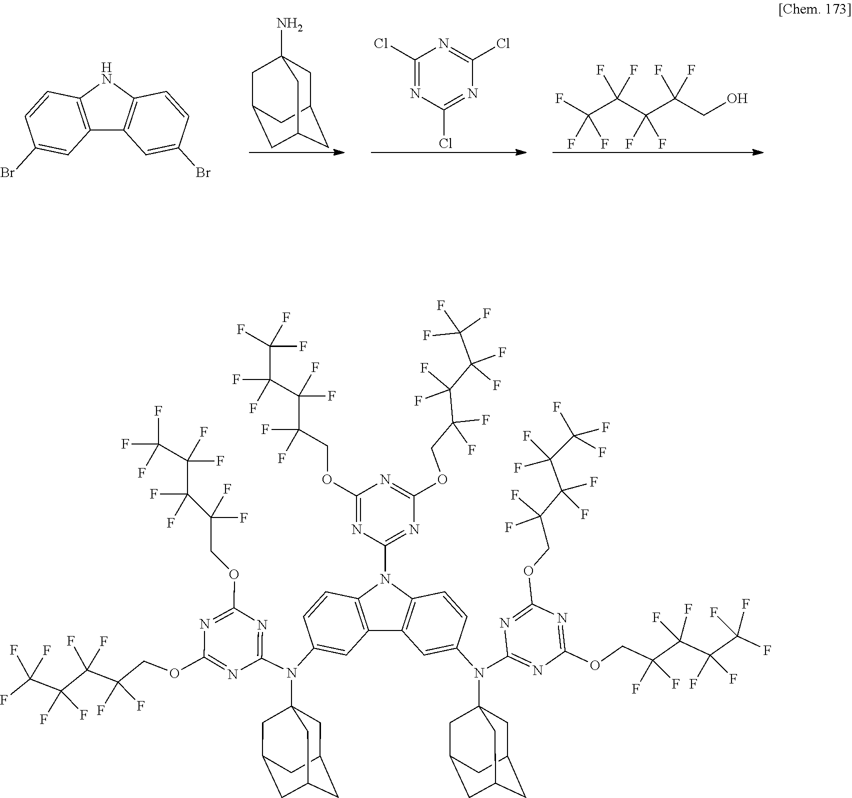 [SMILES: Brc1ccc2[nH]c3ccc(Br)cc3c2c1 ; NC12CC3CC(CC(C3)C1)C2 ; Clc1nc(Cl)nc(Cl)n1 ; OCC(F)(F)C(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(N(c2ccc3c(c2)c2cc(N(c4nc(OCC(F)(F)C(F)(F)C(F)(F)C(F)(F)F)nc(OCC(F)(F)C(F)(F)C(F)(F)C(F)(F)F)n4)C45CC6CC(CC(C6)C4)C5)ccc2n3-c2nc(OCC(F)(F)C(F)(F)C(F)(F)C(F)(F)F)nc(OCC(F)(F)C(F)(F)C(F)(F)C(F)(F)F)n2)C23CC4CC(CC(C4)C2)C3)n1]

In a nitrogen atmosphere, 3,6-dibromo-9H-carbazole (10.0 mmol), 1-aminoadamantane (50.0 mmol), sodium tertiary butoxide (30.0 mmol), 4,5,-bis(diphenylphosphino)-9,9-dimethylxanthene (0.2 mmol), palladium acetate (II) (0.1 mmol) and o-xylene (70 ml) were added and stirred at 140° C. for 6 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce an intermediate S24. Subsequently, in a nitrogen atmosphere, sodium hydride (21.5 mmol) was suspended in tetrahydrofuran (30 ml) and was stirred at 0° C., and S24 (7.0 mmol) was added dropwise to the suspension over 20 minutes. After completion of the dropwise addition, the suspension was returned to room temperature and was stirred for 2 hours. Cyanuric chloride (22 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (48.0 mmol) was suspended in tetrahydrofuran (20 ml) and was stirred at 0° C., and 1H, 1H-nonafluoro-1-pentanol (45.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z252) (yield: 31%).

The compound was identified by FDMS measurement.
FDMS: 2190

### Example 58′ Evaluation of Adhesiveness of Compound (Z252) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z252) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z252) was vapor-deposited, and a 2 mm×1 mm transparent region was formed.

### Example 59 Synthesis of Compound (Z253)

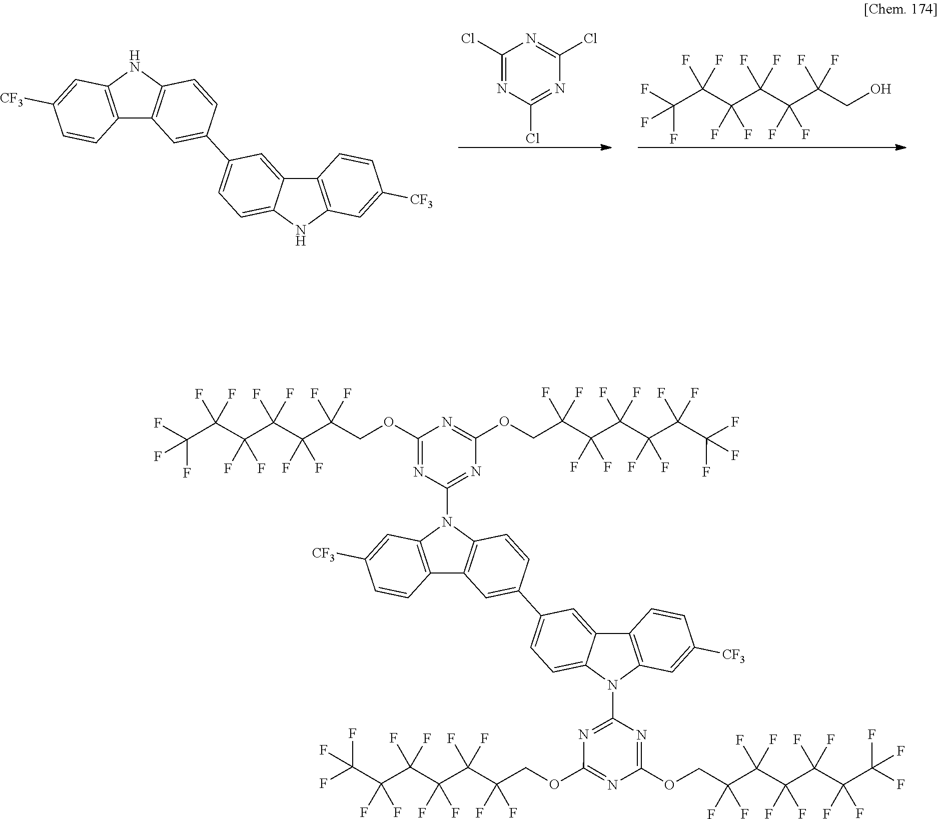 [SMILES: FC(F)(F)c1ccc2c(c1)[nH]c1ccc(-c3ccc4[nH]c5cc(C(F)(F)F)ccc5c4c3)cc12 ; Clc1nc(Cl)nc(Cl)n1 ; OCC(F)(F)C(F)(F)C(F)(F)C(F)(F)C(F)(F)C(F)(F)F ; FC(F)(F)c1ccc2c3cc(-c4ccc5c(c4)c4ccc(C(F)(F)F)cc4n5-c4nc(OCC(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)F)n4)ccc3n(-c3nc(OCC(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)F)n3)c2c1]

In a nitrogen atmosphere, sodium hydride (15.0 mmol) was suspended in tetrahydrofuran (30 ml) and was stirred at 0° C., and 2,2′-ditrifluoromethyl-9H, 9H′-3,3′-dicarbazole (7.0 mmol) was added dropwise to the suspension over 20 minutes. After completion of the dropwise addition, the suspension was returned to room temperature and was stirred for 2 hours. Cyanuric chloride (15.0 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (31.0 mmol) was suspended in tetrahydrofuran (20 ml) and was stirred at 0° C., and 1H, 1H-tridecafluoro-1-heptanol (30.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z253) (yield: 28%).

The compound was identified by FDMS measurement.
FDMS: 2018

### Example 59′ Evaluation of Adhesiveness of Compound (Z253) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z253) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z253) was vapor-deposited, and a 2 mm×1 mm transparent region was formed.

### Example 60′ Evaluation of Adhesiveness of Compound (Z254) to Metal

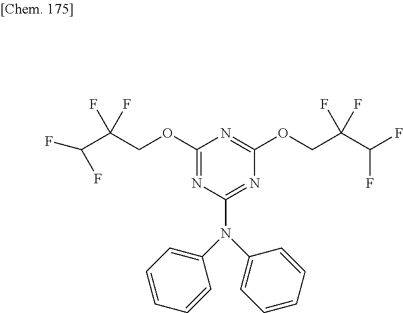 [SMILES: FC(F)C(F)(F)COc1nc(OCC(F)(F)C(F)F)nc(N(c2ccccc2)c2ccccc2)n1]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of a compound (Z254) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z254) was vapor-deposited, and a 2 mm×1 mm transparent region was formed.

### Example 61′ Evaluation of Adhesiveness of Compound (Z255) to Metal

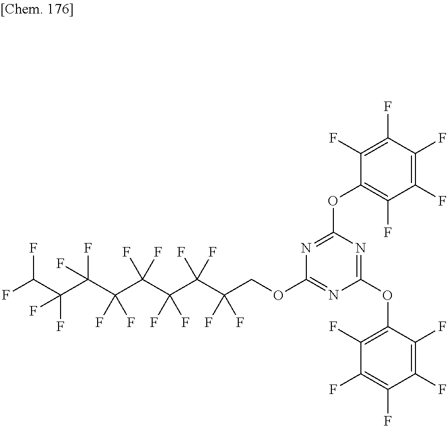 [SMILES: Fc1c(F)c(F)c(Oc2nc(OCC(F)(F)C(F)(F)C(F)(F)C(F)(F)C(F)(F)C(F)(F)C(F)(F)C(F)F)nc(Oc3c(F)c(F)c(F)c(F)c3F)n2)c(F)c1F]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of a compound (Z255) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z255) was vapor-deposited, and a 2 mm×1 mm transparent region was formed.

### Example 62′ Evaluation of Adhesiveness of Compound (Z256) to Metal

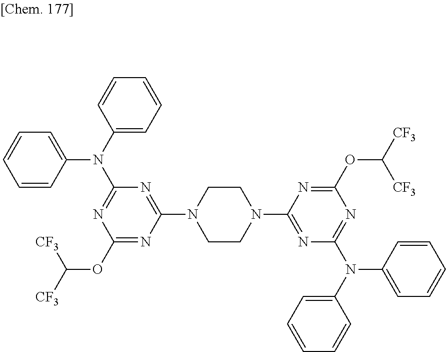 [SMILES: FC(F)(F)C(Oc1nc(N2CCN(c3nc(OC(C(F)(F)F)C(F)(F)F)nc(N(c4ccccc4)c4ccccc4)n3)CC2)nc(N(c2ccccc2)c2ccccc2)n1)C(F)(F)F]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of a compound (Z256) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z256) was vapor-deposited, and a 2 mm×1 mm transparent region was formed.

### Example 63′ Evaluation of Adhesiveness of Compound (Z257) to Metal

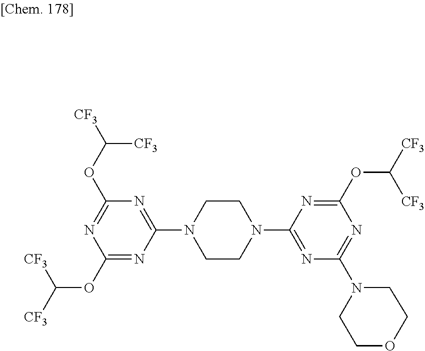 [SMILES: FC(F)(F)C(Oc1nc(OC(C(F)(F)F)C(F)(F)F)nc(N2CCN(c3nc(OC(C(F)(F)F)C(F)(F)F)nc(N4CCOCC4)n3)CC2)n1)C(F)(F)F]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of a compound (Z257) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z257) was vapor-deposited, and a 2 mm×1 mm transparent region was formed.

### Example 64′ Evaluation of Adhesiveness of Compound (Z258) to Metal

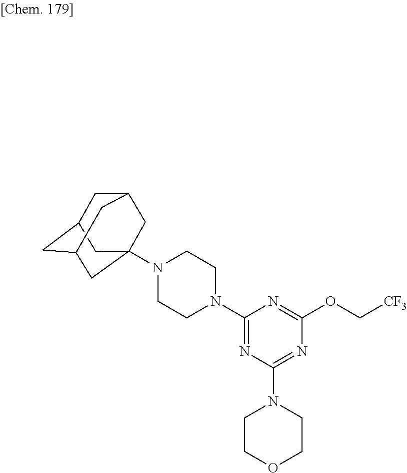 [SMILES: FC(F)(F)COc1nc(N2CCOCC2)nc(N2CCN(C34CC5CC(CC(C5)C3)C4)CC2)n1]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of a compound (Z258) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z258) was vapor-deposited, and a 2 mm×1 mm transparent region was formed.

### Example 65′ Evaluation of Adhesiveness of Compound (Z259) to Metal

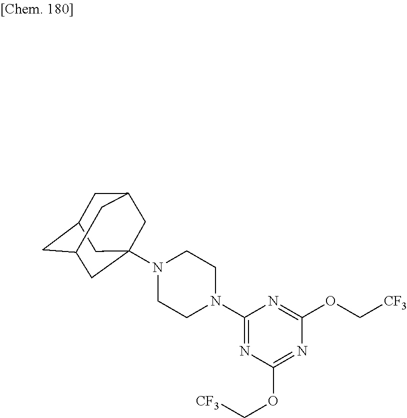 [SMILES: FC(F)(F)COc1nc(OCC(F)(F)F)nc(N2CCN(C34CC5CC(CC(C5)C3)C4)CC2)n1]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of a compound (Z259) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z259) was vapor-deposited, and a 2 mm×1 mm transparent region was formed.

### Example 66′ Evaluation of Adhesiveness of Compound (Z260) to Metal

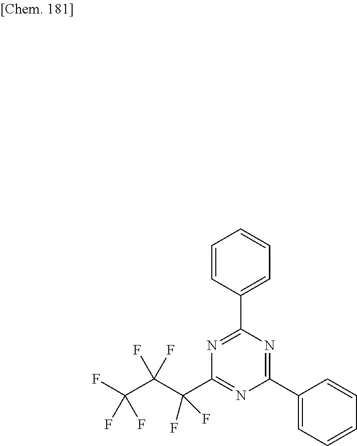 [SMILES: FC(F)(F)C(F)(F)C(F)(F)c1nc(-c2ccccc2)nc(-c2ccccc2)n1]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z260) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z260) was vapor-deposited, and a 2 mm×1 mm transparent region was formed.

### Example 67′ Evaluation of Adhesiveness of Compound (Z261) to Metal

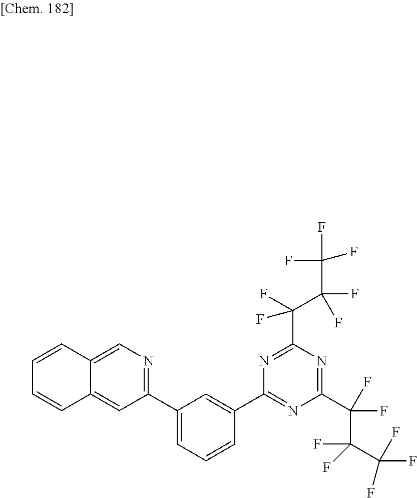 [SMILES: FC(F)(F)C(F)(F)C(F)(F)c1nc(-c2cccc(-c3cc4ccccc4cn3)c2)nc(C(F)(F)C(F)(F)C(F)(F)F)n1]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of a compound (Z261) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z261) was vapor-deposited, and a 2 mm×1 mm transparent region was formed.

### Example 68′ Evaluation of Adhesiveness of Compound (Z262) to Metal

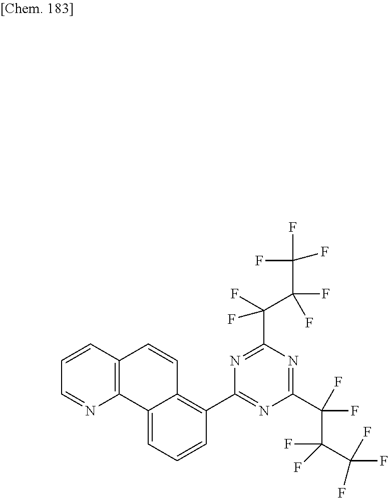 [SMILES: FC(F)(F)C(F)(F)C(F)(F)c1nc(-c2cccc3c2ccc2cccnc23)nc(C(F)(F)C(F)(F)C(F)(F)F)n1]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of a compound (Z262) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z262) was vapor-deposited, and a 2 mm×1 mm transparent region was formed.

### Example 69′ Evaluation of Adhesiveness of Compound (Z263) to Metal

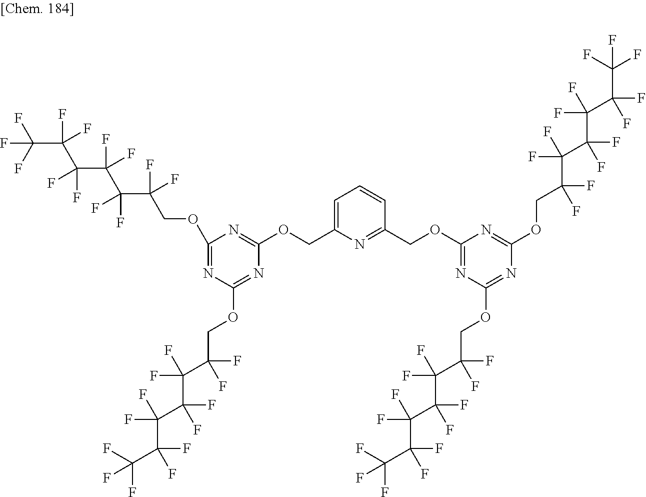 [SMILES: FC(F)(F)C(F)(F)C(F)(F)C(F)(F)C(F)(F)C(F)(F)COc1nc(OCc2cccc(COc3nc(OCC(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)F)n3)n2)nc(OCC(F)(F)C(F)(F)C(F)(F)C(F)(F)C(F)(F)C(F)(F)F)n1]

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of a compound (Z263) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z263) was vapor-deposited, and a 2 mm×1 mm transparent region was formed.

### Example 70 Synthesis of Compound (Z264)

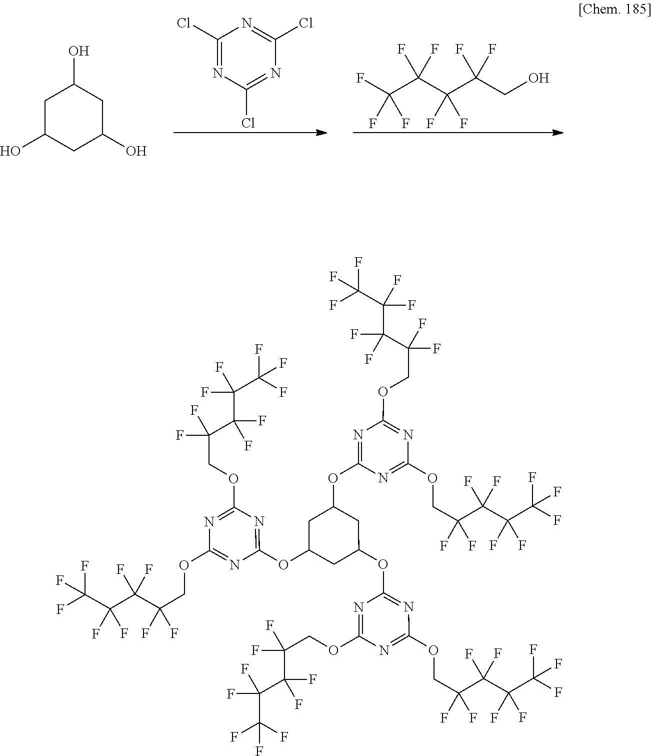 [SMILES: OC1CC(O)CC(O)C1 ; Clc1nc(Cl)nc(Cl)n1 ; OCC(F)(F)C(F)(F)C(F)(F)C(F)(F)F ; FC(F)(F)C(F)(F)C(F)(F)C(F)(F)COc1nc(OCC(F)(F)C(F)(F)C(F)(F)C(F)(F)F)nc(OC2CC(Oc3nc(OCC(F)(F)C(F)(F)C(F)(F)C(F)(F)F)nc(OCC(F)(F)C(F)(F)C(F)(F)C(F)(F)F)n3)CC(Oc3nc(OCC(F)(F)C(F)(F)C(F)(F)C(F)(F)F)nc(OCC(F)(F)C(F)(F)C(F)(F)C(F)(F)F)n3)C2)n1]

In a nitrogen atmosphere, sodium hydride (16.0 mmol) was suspended in tetrahydrofuran (30 ml) and was stirred at 0° C., and 1,3,5-cyclohexanetriol (5.0 mmol) was added dropwise to the suspension over 20 minutes. After completion of the dropwise addition, the suspension was returned to room temperature and was stirred for 2 hours. Cyanuric chloride (16.0 mmol) dissolved in tetrahydrofuran (30 ml) was then added dropwise over 20 minutes. The dropwise addition was followed by stirring at 60° C. for 3 hours. Next, in the reactor cooled to 0° C., in a nitrogen atmosphere, sodium hydride (33.0 mmol) was suspended in tetrahydrofuran (20 ml) and was stirred at 0° C., and 1H, 1H-nonafluoro-1-pentanol (32.0 mmol) was added dropwise to the suspension over 20 minutes. After completion of the dropwise addition, the reaction solution returned to room temperature and stirred for 2 hours was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z264) (yield: 22%).

The compound was identified by FDMS measurement.
FDMS: 1857

### Example 70′ Evaluation of Adhesiveness of Compound (Z264) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z264) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z264) was vapor-deposited, and a 2 mm×1 mm transparent region was formed.

### Example 71 Synthesis of Compound (Z265)

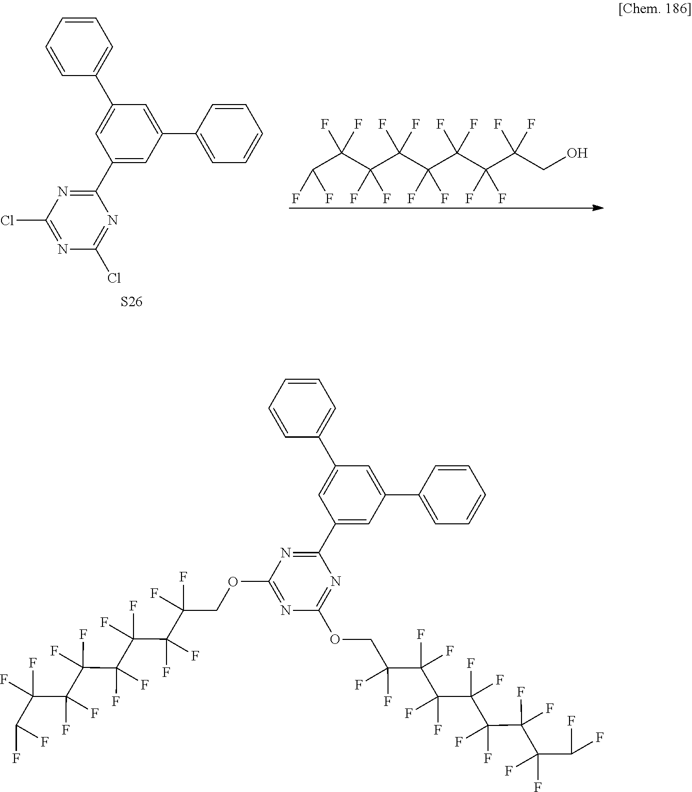 [SMILES: Clc1nc(Cl)nc(-c2cc(-c3ccccc3)cc(-c3ccccc3)c2)n1 ; OCC(F)(F)C(F)(F)C(F)(F)C(F)(F)C(F)(F)C(F)(F)C(F)(F)C(F)F ; "S26" ; FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(-c2cc(-c3ccccc3)cc(-c3ccccc3)c2)n1]

In a nitrogen atmosphere, sodium hydride (12.0 mmol) was suspended in tetrahydrofuran (20 ml) and was stirred at 0° C., and 1H, 1H, 9H-hexadecafluoro-1-nonanol (11.0 mmol) was added dropwise to the suspension over 20 minutes. After completion of the dropwise addition, the temperature was returned to room temperature, and S26 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z265) (yield: 81%).

The compound was identified by FDMS measurement.
FDMS: 1169

### Example 71′ Evaluation of Adhesiveness of Compound (Z265) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z265) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z265) was vapor-deposited, and a 2 mm×1 mm transparent region was formed.

### Example 72 Synthesis of Compound (Z266)

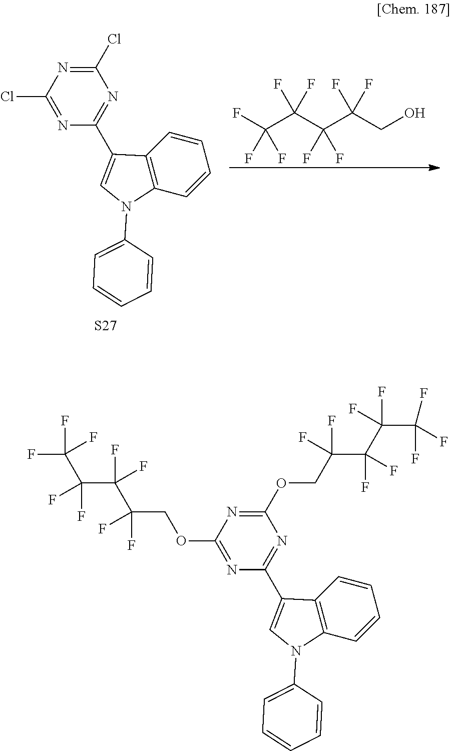

In a nitrogen atmosphere, sodium hydride (12.0 mmol) was suspended in tetrahydrofuran (20 ml) and was stirred at 0° C., and 1H, 1H-nonafluoro-1-pentanol (11.0 mmol) was added dropwise to the suspension over 20 minutes. After completion of the dropwise addition, the temperature was returned to room temperature, and S27 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z266) (yield: 69%).

The compound was identified by FDMS measurement.
FDMS: 768

### Example 72′ Evaluation of Adhesiveness of Compound (Z266) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z266) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z266) was vapor-deposited, and a 2 mm×1 mm transparent region was formed.

### Example 73 Synthesis of Compound (Z267)

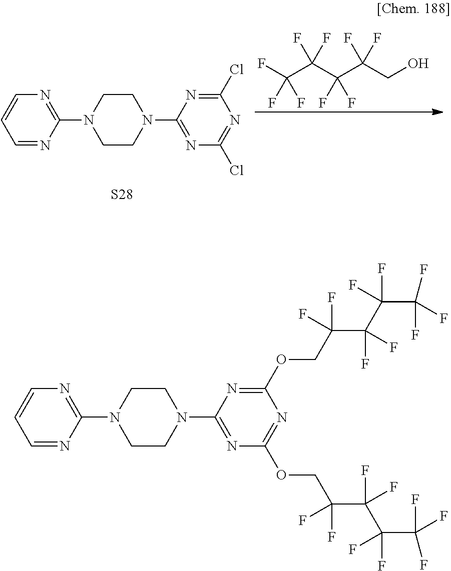

In a nitrogen atmosphere, sodium hydride (12.0 mmol) was suspended in tetrahydrofuran (20 ml) and was stirred at 0° C., and 1H, 1H-nonafluoro-1-pentanol (11.0 mmol) was added dropwise to the suspension over 20 minutes. After completion of the dropwise addition, the temperature was returned to room temperature, and S28 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z267) (yield: 75%).

The compound was identified by FDMS measurement.
FDMS: 739

### Example 73′ Evaluation of Adhesiveness of Compound (Z267) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z267) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z267) was vapor-deposited, and a 2 mm×1 mm transparent region was formed.

### Example 74 Synthesis of Compound (Z268)

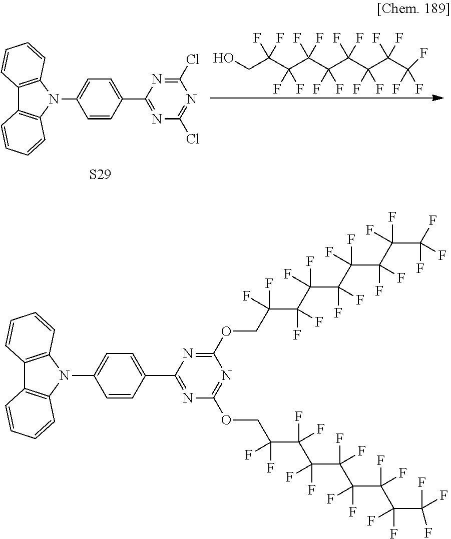 [SMILES: Clc1nc(Cl)nc(-c2ccc(-n3c4ccccc4c4ccccc43)cc2)n1 ; OCC(F)(F)C(F)(F)C(F)(F)C(F)(F)C(F)(F)C(F)(F)C(F)(F)C(F)(F)F ; "S29" ; FC(F)(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)(F)F)nc(-c2ccc(-n3c4ccccc4c4ccccc43)cc2)n1]

In a nitrogen atmosphere, sodium hydride (12.0 mmol) was suspended in tetrahydrofuran (20 ml) and was stirred at 0° C., and 1H, 1H-heptadecafluoro-1-nonanol (11.0 mmol) was added dropwise to the suspension over 20 minutes. After completion of the dropwise addition, the temperature was returned to room temperature, and S29 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z268) (yield: 69%).

The compound was identified by FDMS measurement.
FDMS: 739

### Example 74′ Evaluation of Adhesiveness of Compound (Z268) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z268) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z268) was vapor-deposited, and a 2 mm×1 mm transparent region was formed.

### Example 75 Synthesis of Compound (Z269)

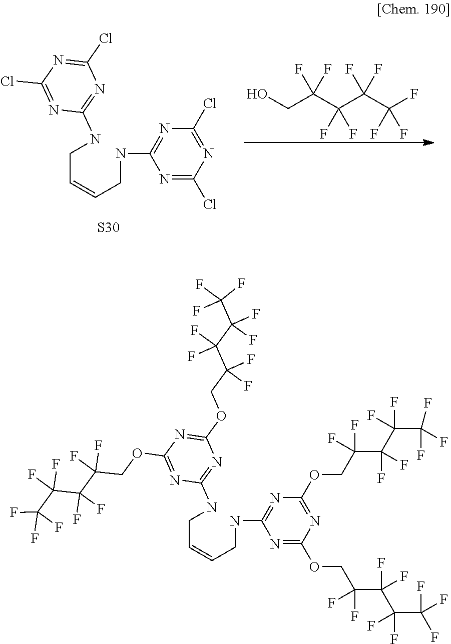 [SMILES: Clc1nc(Cl)nc(NC/C=C\CNc2nc(Cl)nc(Cl)n2)n1 ; OCC(F)(F)C(F)(F)C(F)(F)C(F)(F)F ; "S30" ; FC(F)(F)C(F)(F)C(F)(F)C(F)(F)COc1nc(NC/C=C\CNc2nc(OCC(F)(F)C(F)(F)C(F)(F)C(F)(F)F)nc(OCC(F)(F)C(F)(F)C(F)(F)C(F)(F)F)n2)nc(OCC(F)(F)C(F)(F)C(F)(F)C(F)(F)F)n1]

In a nitrogen atmosphere, sodium hydride (24.0 mmol) was suspended in tetrahydrofuran (20 ml) and was stirred at 0° C., and 1H, 1H-nonafluoro-1-pentanol (22.0 mmol) was added dropwise to the suspension over 20 minutes. After completion of the dropwise addition, the temperature was returned to room temperature, and S30 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z269) (yield: 41%).

The compound was identified by FDMS measurement.
FDMS: 1234

### Example 75′ Evaluation of Adhesiveness of Compound (Z269) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z269) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z269) was vapor-deposited, and a 2 mm×1 mm transparent region was formed.

### Example 76 Synthesis of Compound (Z270)

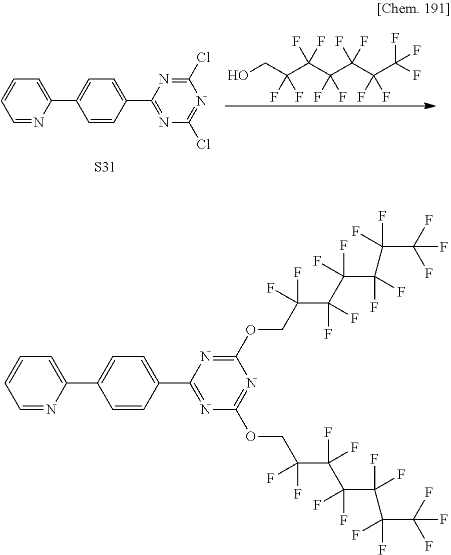 [SMILES: Clc1nc(Cl)nc(-c2ccc(-c3ccccn3)cc2)n1 ; OCC(F)(F)C(F)(F)C(F)(F)C(F)(F)C(F)(F)C(F)(F)F ; "S31" ; FC(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)F)nc(-c2ccc(-c3ccccn3)cc2)n1]

In a nitrogen atmosphere, sodium hydride (12.0 mmol) was suspended in tetrahydrofuran (20 ml) and was stirred at 0° C., and 1H, 1H-tridecafluoro-1-heptanol (11.0 mmol) was added dropwise to the suspension over 20 minutes. After completion of the dropwise addition, the temperature was returned to room temperature, and S31 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z270) (yield: 68%).

The compound was identified by FDMS measurement.
FDMS: 930

### Example 76′ Evaluation of Adhesiveness of Compound (Z270) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z270) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z270) was vapor-deposited, and a 2 mm×1 mm transparent region was formed.

### Example 77 Synthesis of Compound (Z271)

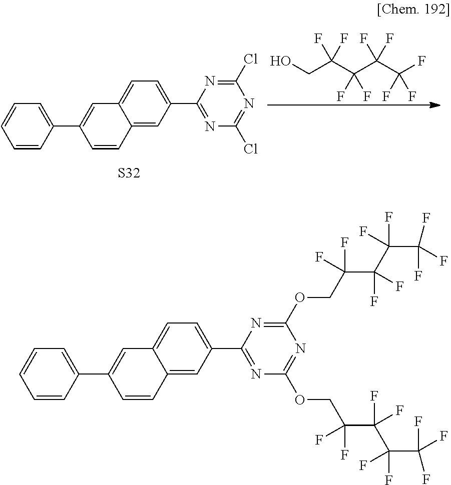

In a nitrogen atmosphere, sodium hydride (12.0 mmol) was suspended in tetrahydrofuran (20 ml) and was stirred at 0° C., and 1H, 1H-nonafluoro-1-pentanol (11.0 mmol) was added dropwise to the suspension over 20 minutes. After completion of the dropwise addition, the temperature was returned to room temperature, and S32 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z271) (yield: 88%).

The compound was identified by FDMS measurement.
FDMS: 779

### Example 77′ Evaluation of Adhesiveness of Compound (Z271) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z271) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z271) was vapor-deposited, and a 2 mm×1 mm transparent region was formed.

### Example 78 Synthesis of Compound (Z272)

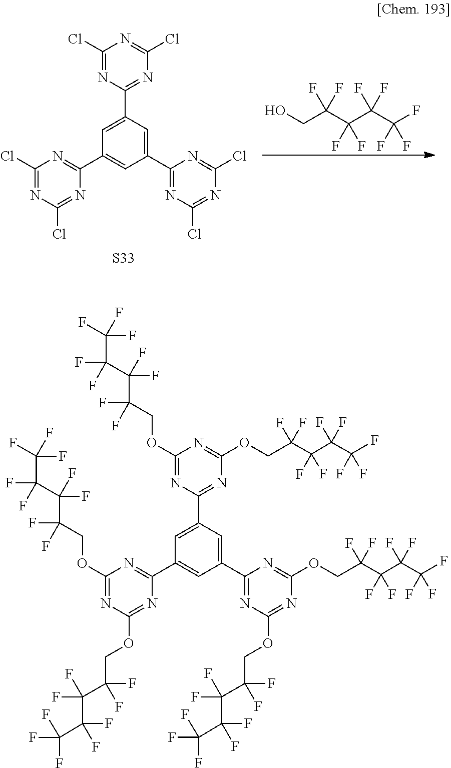 [SMILES: Clc1nc(Cl)nc(-c2cc(-c3nc(Cl)nc(Cl)n3)cc(-c3nc(Cl)nc(Cl)n3)c2)n1 ; OCC(F)(F)C(F)(F)C(F)(F)C(F)(F)F ; "S33" ; FC(F)(F)C(F)(F)C(F)(F)C(F)(F)COc1nc(OCC(F)(F)C(F)(F)C(F)(F)C(F)(F)F)nc(-c2cc(-c3nc(OCC(F)(F)C(F)(F)C(F)(F)C(F)(F)F)nc(OCC(F)(F)C(F)(F)C(F)(F)C(F)(F)F)n3)cc(-c3nc(OCC(F)(F)C(F)(F)C(F)(F)C(F)(F)F)nc(OCC(F)(F)C(F)(F)C(F)(F)C(F)(F)F)n3)c2)n1]

In a nitrogen atmosphere, sodium hydride (36.0 mmol) was suspended in tetrahydrofuran (20 ml) and was stirred at 0° C., and 1H, 1H-nonafluoro-1-pentanol (33.0 mmol) was added dropwise to the suspension over 20 minutes. After completion of the dropwise addition, the temperature was returned to room temperature, and S33 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z272) (yield: 35%).

The compound was identified by FDMS measurement.
FDMS: 1803

### Example 78′ Evaluation of Adhesiveness of Compound (Z272) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z272) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z272) was vapor-deposited, and a 2 mm×1 mm transparent region was formed.

### Example 79 Synthesis of Compound (Z273)

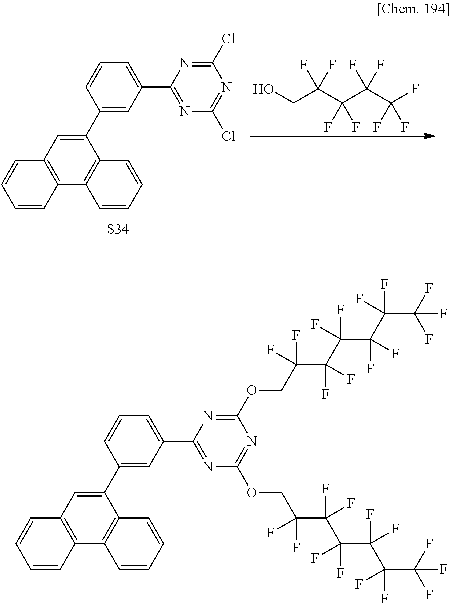 [SMILES: Clc1nc(Cl)nc(-c2cccc(-c3cc4ccccc4c4ccccc34)c2)n1 ; OCC(F)(F)C(F)(F)C(F)(F)C(F)(F)F ; "S34" ; FC(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)F)nc(-c2cccc(-c3cc4ccccc4c4ccccc34)c2)n1]

In a nitrogen atmosphere, sodium hydride (12.0 mmol) was suspended in tetrahydrofuran (20 ml) and was stirred at 0° C., and 1H, 1H-tridecafluoro-1-heptanol (11.0 mmol) was added dropwise to the suspension over 20 minutes. After completion of the dropwise addition, the temperature was returned to room temperature, and S34 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z273) (yield: 69%).

The compound was identified by FDMS measurement.
FDMS: 1029

### Example 79′ Evaluation of Adhesiveness of Compound (Z273) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z273) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z273) was vapor-deposited, and a 2 mm×1 mm transparent region was formed.

### Example 80 Synthesis of Compound (Z274)

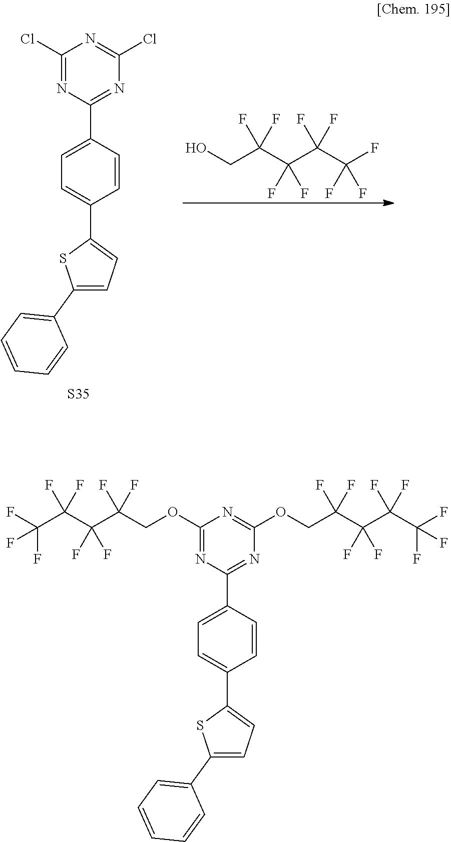 [SMILES: Clc1nc(Cl)nc(-c2ccc(-c3ccc(-c4ccccc4)s3)cc2)n1 ; OCC(F)(F)C(F)(F)C(F)(F)C(F)(F)F ; "S35" ; FC(F)(F)C(F)(F)C(F)(F)C(F)(F)COc1nc(OCC(F)(F)C(F)(F)C(F)(F)C(F)(F)F)nc(-c2ccc(-c3ccc(-c4ccccc4)s3)cc2)n1]

In a nitrogen atmosphere, sodium hydride (12.0 mmol) was suspended in tetrahydrofuran (20 ml) and was stirred at 0° C., and 1H, 1H-nonafluoro-1-pentanol (11.0 mmol) was added dropwise to the suspension over 20 minutes. After completion of the dropwise addition, the temperature was returned to room temperature, and S35 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z274) (yield: 71%).

The compound was identified by FDMS measurement.
FDMS: 811

### Example 80′ Evaluation of Adhesiveness of Compound (Z274) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z274) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z274) was vapor-deposited, and a 2 mm×1 mm transparent region was formed.

### Example 81 Synthesis of Compound (Z275)

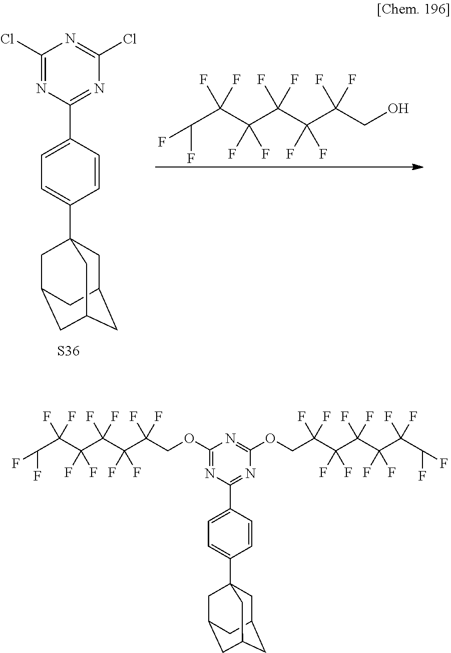 [SMILES: Clc1nc(Cl)nc(-c2ccc(C34CC5CC(CC(C5)C3)C4)cc2)n1 ; OCC(F)(F)C(F)(F)C(F)(F)C(F)(F)C(F)(F)C(F)F ; "S36" ; FC(F)C(F)(F)C(F)(F)C(F)(F)C(F)(F)C(F)(F)COc1nc(OCC(F)(F)C(F)(F)C(F)(F)C(F)(F)C(F)(F)C(F)F)nc(-c2ccc(C34CC5CC(CC(C5)C3)C4)cc2)n1]

In a nitrogen atmosphere, sodium hydride (12.0 mmol) was suspended in tetrahydrofuran (20 ml) and was stirred at 0° C., and 1H, 1H, 7H-dodecafluoro-1-heptanol (11.0 mmol) was added dropwise to the suspension over 20 minutes. After completion of the dropwise addition, the temperature was returned to room temperature, and S36 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z275) (yield: 80%).

The compound was identified by FDMS measurement.
FDMS: 951

### Example 81′ Evaluation of Adhesiveness of Compound (Z275) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z275) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z275) was vapor-deposited, and a 2 mm×1 mm transparent region was formed.

### Example 82 Synthesis of Compound (Z276)

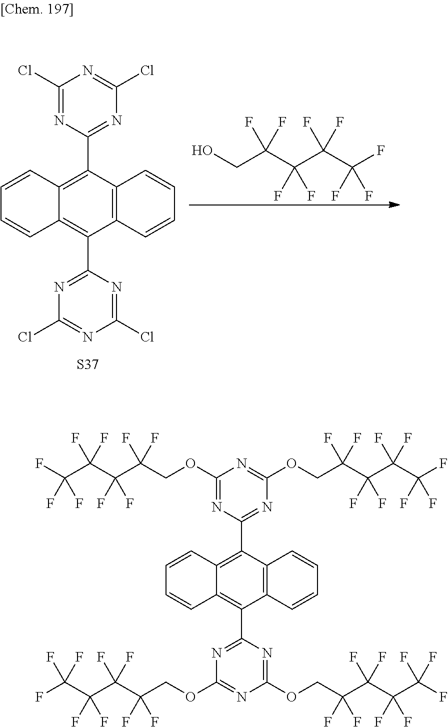 [SMILES: Clc1nc(Cl)nc(-c2c3ccccc3c(-c3nc(Cl)nc(Cl)n3)c3ccccc23)n1 ; OCC(F)(F)C(F)(F)C(F)(F)C(F)(F)F ; "S37" ; FC(F)(F)C(F)(F)C(F)(F)C(F)(F)COc1nc(OCC(F)(F)C(F)(F)C(F)(F)C(F)(F)F)nc(-c2c3ccccc3c(-c3nc(OCC(F)(F)C(F)(F)C(F)(F)C(F)(F)F)nc(OCC(F)(F)C(F)(F)C(F)(F)C(F)(F)F)n3)c3ccccc23)n1]

In a nitrogen atmosphere, sodium hydride (24.0 mmol) was suspended in tetrahydrofuran (20 ml) and was stirred at 0° C., and 1H, 1H-nonafluoro-1-pentanol (22.0 mmol) was added dropwise to the suspension over 20 minutes. After completion of the dropwise addition, the temperature was returned to room temperature, and S37 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z276) (yield: 49%).

The compound was identified by FDMS measurement.
FDMS: 1328

### Example 82′ Evaluation of Adhesiveness of Compound (Z276) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z276) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z276) was vapor-deposited, and a 2 mm×1 mm transparent region was formed.

### Example 83 Synthesis of Compound (Z277)

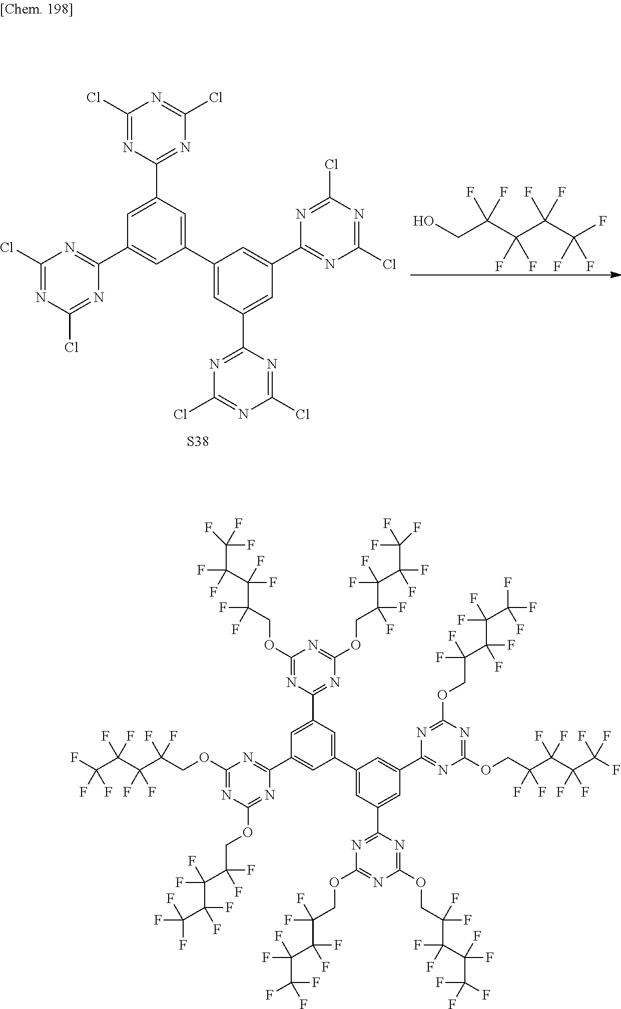 [SMILES: Clc1nc(Cl)nc(-c2cc(-c3cc(-c4nc(Cl)nc(Cl)n4)cc(-c4nc(Cl)nc(Cl)n4)c3)cc(-c3nc(Cl)nc(Cl)n3)c2)n1 ; OCC(F)(F)C(F)(F)C(F)(F)C(F)(F)F ; "S38" ; FC(F)(F)C(F)(F)C(F)(F)C(F)(F)COc1nc(OCC(F)(F)C(F)(F)C(F)(F)C(F)(F)F)nc(-c2cc(-c3cc(-c4nc(OCC(F)(F)C(F)(F)C(F)(F)C(F)(F)F)nc(OCC(F)(F)C(F)(F)C(F)(F)C(F)(F)F)n4)cc(-c4nc(OCC(F)(F)C(F)(F)C(F)(F)C(F)(F)F)nc(OCC(F)(F)C(F)(F)C(F)(F)C(F)(F)F)n4)c3)cc(-c3nc(OCC(F)(F)C(F)(F)C(F)(F)C(F)(F)F)nc(OCC(F)(F)C(F)(F)C(F)(F)C(F)(F)F)n3)c2)n1]

In a nitrogen atmosphere, sodium hydride (48.0 mmol) was suspended in tetrahydrofuran (20 ml) and was stirred at 0° C., and 1H, 1H-nonafluoro-1-pentanol (44.0 mmol) was added dropwise to the suspension over 20 minutes. After completion of the dropwise addition, the temperature was returned to room temperature, and S38 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z277) (yield: 22%).

The compound was identified by FDMS measurement.
FDMS: 811

### Example 83′ Evaluation of Adhesiveness of Compound (Z277) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z277) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z277) was vapor-deposited, and a 2 mm×1 mm transparent region was formed.

### Example 84 Synthesis of Compound (Z278)

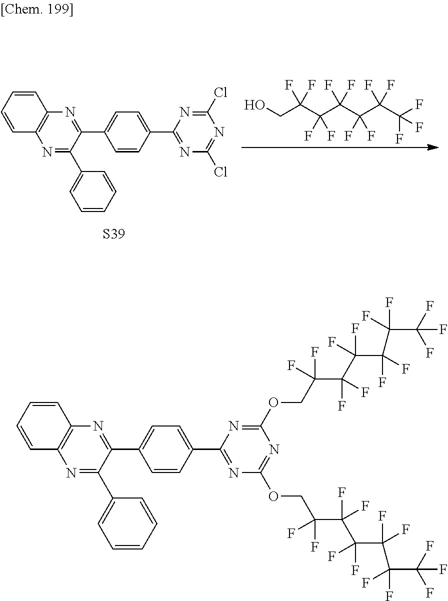 [SMILES: Clc1nc(Cl)nc(-c2ccc(-c3nc4ccccc4nc3-c3ccccc3)cc2)n1 ; OCC(F)(F)C(F)(F)C(F)(F)C(F)(F)C(F)(F)C(F)(F)F ; "S39" ; FC(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)F)nc(-c2ccc(-c3nc4ccccc4nc3-c3ccccc3)cc2)n1]

In a nitrogen atmosphere, sodium hydride (12.0 mmol) was suspended in tetrahydrofuran (20 ml) and was stirred at 0° C., and 1H, 1H-tridecafluoro-1-heptanol (11.0 mmol) was added dropwise to the suspension over 20 minutes. After completion of the dropwise addition, the temperature was returned to room temperature, and S39 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z278) (yield: 48%).

The compound was identified by FDMS measurement.
FDMS: 1029

### Example 84′ Evaluation of Adhesiveness of Compound (Z278) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z278) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z278) was vapor-deposited, and a 2 mm×1 mm transparent region was formed.

### Example 85 Synthesis of Compound (Z279)

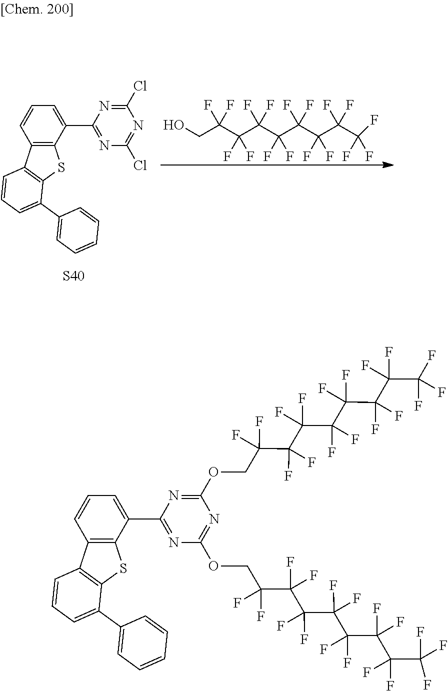 [SMILES: Clc1nc(Cl)nc(-c2cccc3c2sc2c(-c4ccccc4)cccc23)n1 ; OCC(F)(F)C(F)(F)C(F)(F)C(F)(F)C(F)(F)C(F)(F)C(F)(F)C(F)(F)F ; "S40" ; FC(F)(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)(F)F)nc(-c2cccc3c2sc2c(-c4ccccc4)cccc23)n1]

In a nitrogen atmosphere, sodium hydride (12.0 mmol) was suspended in tetrahydrofuran (20 ml) and was stirred at 0° C., and 1H, 1H-heptadecafluoro-1-nonanol (11.0 mmol) was added dropwise to the suspension over 20 minutes. After completion of the dropwise addition, the temperature was returned to room temperature, and S40 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z279) (yield: 49%).

The compound was identified by FDMS measurement.
FDMS: 1235

### Example 85′ Evaluation of Adhesiveness of Compound (Z279) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z279) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z279) was vapor-deposited, and a 2 mm×1 mm transparent region was formed.

### Example 86 Synthesis of Compound (Z280)

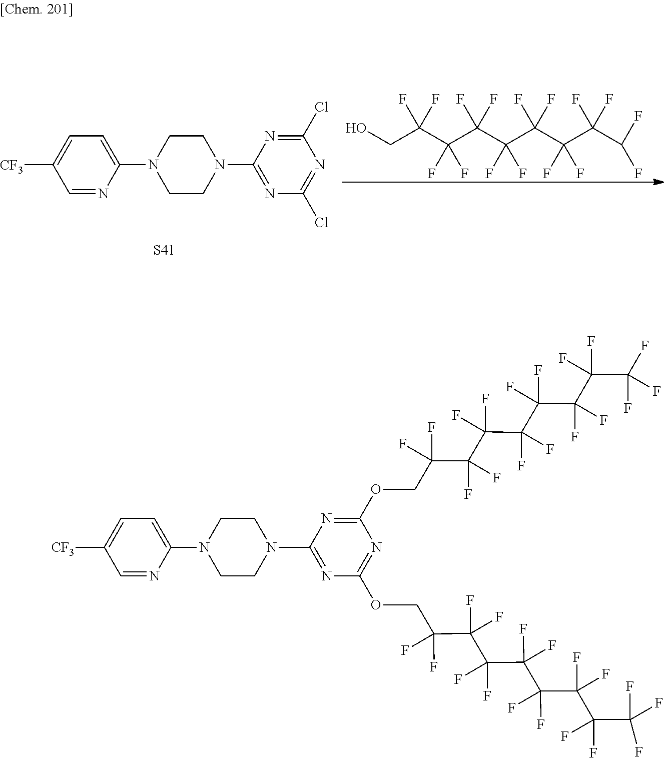 [SMILES: FC(F)(F)c1ccc(N2CCN(c3nc(Cl)nc(Cl)n3)CC2)nc1 ; OCC(F)(F)C(F)(F)C(F)(F)C(F)(F)C(F)(F)C(F)(F)C(F)(F)C(F)F ; "S41" ; FC(F)(F)c1ccc(N2CCN(c3nc(OCC(F)(F)C(F)(F)C(F)(F)C(F)(F)C(F)(F)C(F)(F)C(F)(F)C(F)(F)F)nc(OCC(F)(F)C(F)(F)C(F)(F)C(F)(F)C(F)(F)C(F)(F)C(F)(F)C(F)(F)F)n3)CC2)nc1]

In a nitrogen atmosphere, sodium hydride (12.0 mmol) was suspended in tetrahydrofuran (20 ml) and was stirred at 0° C., and 1H, 1H-heptadecafluoro-1-nonanol (11.0 mmol) was added dropwise to the suspension over 20 minutes. After completion of the dropwise addition, the temperature was returned to room temperature, and S41 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z280) (yield: 61%).

The compound was identified by FDMS measurement.
FDMS: 1206

### Example 86′ Evaluation of Adhesiveness of Compound (Z280) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z280) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z280) was vapor-deposited, and a 2 mm×1 mm transparent region was formed.

### Example 87 Synthesis of Compound (Z281)

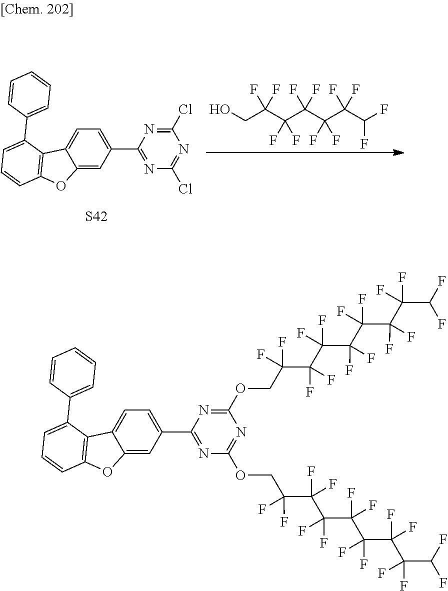 [SMILES: Clc1nc(Cl)nc(-c2ccc3c(c2)oc2cccc(-c4ccccc4)c23)n1 ; OCC(F)(F)C(F)(F)C(F)(F)C(F)(F)C(F)(F)C(F)F ; "S42" ; FC(F)C(F)(F)C(F)(F)C(F)(F)C(F)(F)C(F)(F)C(F)(F)C(F)(F)COc1nc(OCC(F)(F)C(F)(F)C(F)(F)C(F)(F)C(F)(F)C(F)(F)C(F)(F)C(F)F)nc(-c2ccc3c(c2)oc2cccc(-c4ccccc4)c23)n1]

In a nitrogen atmosphere, sodium hydride (12.0 mmol) was suspended in tetrahydrofuran (20 ml) and was stirred at 0° C., and 1H, 1H, 7H-dodecafluoro-1-heptanol (11.0 mmol) was added dropwise to the suspension over 20 minutes. After completion of the dropwise addition, the temperature was returned to room temperature, and S42 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z281) (yield: 58%).

The compound was identified by FDMS measurement.
FDMS: 983

### Example 87′ Evaluation of Adhesiveness of Compound (Z281) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z281) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z281) was vapor-deposited, and a 2 mm×1 mm transparent region was formed.

### Example 88 Synthesis of Compound (Z282)

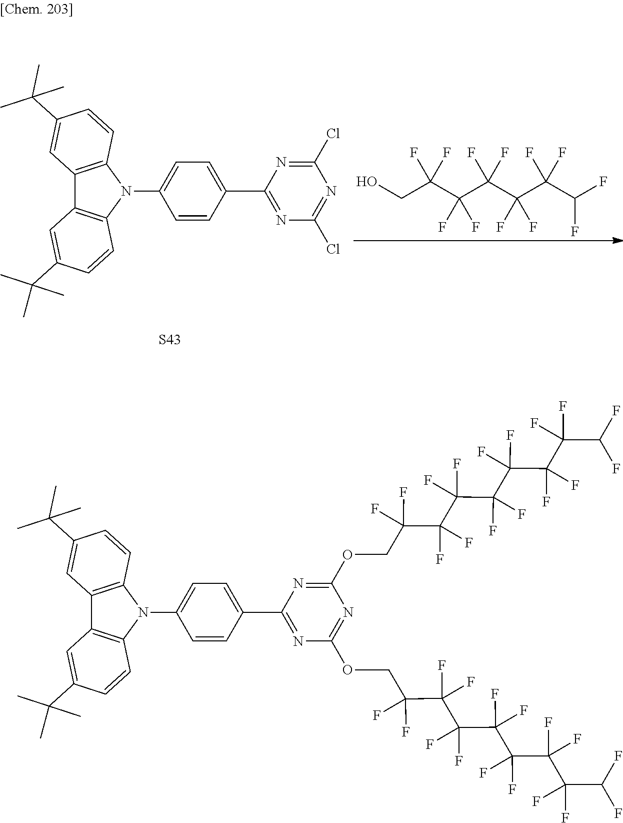

In a nitrogen atmosphere, sodium hydride (12.0 mmol) was suspended in tetrahydrofuran (20 ml) and was stirred at 0° C., and 1H, 1H, 7H-dodecafluoro-1-heptanol (11.0 mmol) was added dropwise to the suspension over 20 minutes. After completion of the dropwise addition, the temperature was returned to room temperature, and S43 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z282) (yield: 69%).

The compound was identified by FDMS measurement.
FDMS: 1094

### Example 88′ Evaluation of Adhesiveness of Compound (Z282) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z282) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z282) was vapor-deposited, and a 2 mm×1 mm transparent region was formed.

### Example 89 Synthesis of Compound (Z283)

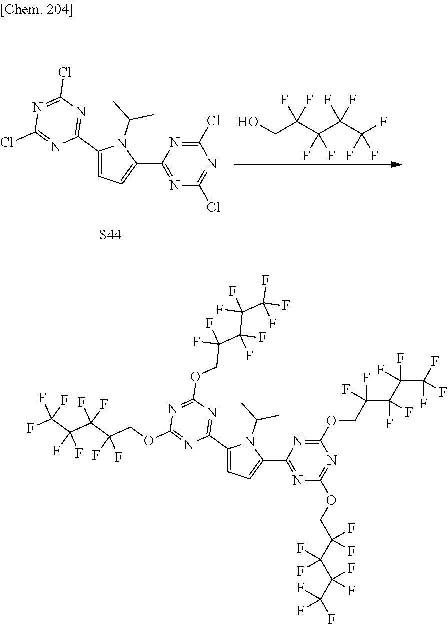 [SMILES: CC(C)n1c(-c2nc(Cl)nc(Cl)n2)ccc1-c1nc(Cl)nc(Cl)n1 ; OCC(F)(F)C(F)(F)C(F)(F)C(F)(F)F ; "S44" ; CC(C)n1c(-c2nc(OCC(F)(F)C(F)(F)C(F)(F)C(F)(F)F)nc(OCC(F)(F)C(F)(F)C(F)(F)C(F)(F)F)n2)ccc1-c1nc(OCC(F)(F)C(F)(F)C(F)(F)C(F)(F)F)nc(OCC(F)(F)C(F)(F)C(F)(F)C(F)(F)F)n1]

In a nitrogen atmosphere, sodium hydride (24.0 mmol) was suspended in tetrahydrofuran (20 ml) and was stirred at 0° C., and 1H, 1H-nonafluoro-1-pentanol (22.0 mmol) was added dropwise to the suspension over 20 minutes. After completion of the dropwise addition, the temperature was returned to room temperature, and S44 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z283) (yield: 49%).

The compound was identified by FDMS measurement.
FDMS: 1259

### Example 89′ Evaluation of Adhesiveness of Compound (Z283) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z283) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z283) was vapor-deposited, and a 2 mm×1 mm transparent region was formed.

### Example 90 Synthesis of Compound (Z284)

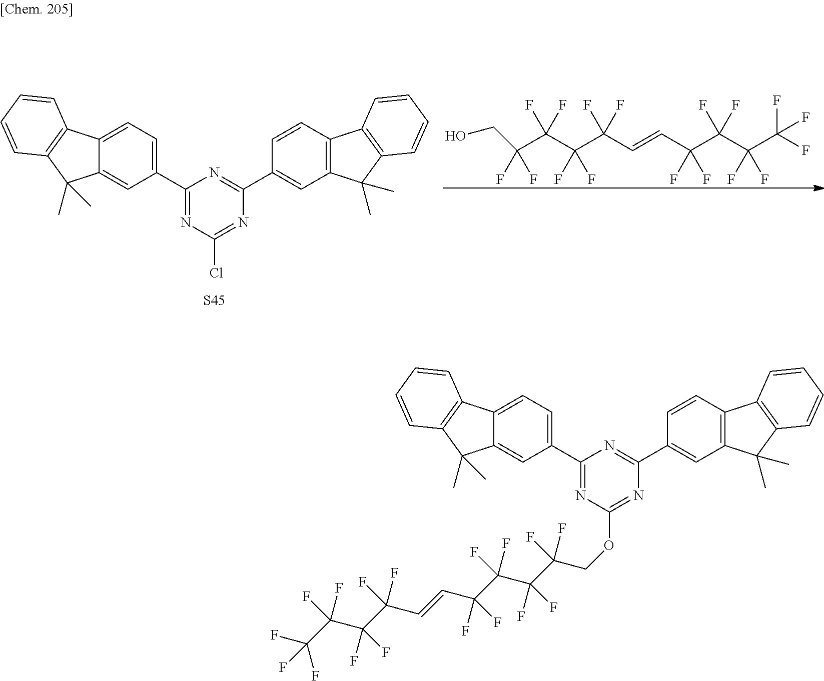

In a nitrogen atmosphere, sodium hydride (6.0 mmol) was suspended in tetrahydrofuran (20 ml) and was stirred at 0° C., and 2, 2, 3, 3, 4, 4, 5, 5, 8, 8, 9, 9, 10, 10, 11, 11, 11-heptadecafluoro-6-undecen-1-ol (5.5 mmol) was added dropwise to the suspension over 20 minutes. After completion of the dropwise addition, the temperature was returned to room temperature, and S45 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z284) (yield: 22%).

The compound was identified by FDMS measurement.
FDMS: 939

### Example 90′ Evaluation of Adhesiveness of Compound (Z284) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z284) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z284) was vapor-deposited, and a 2 mm×1 mm transparent region was formed.

### Example 91 Synthesis of Compound (Z285)

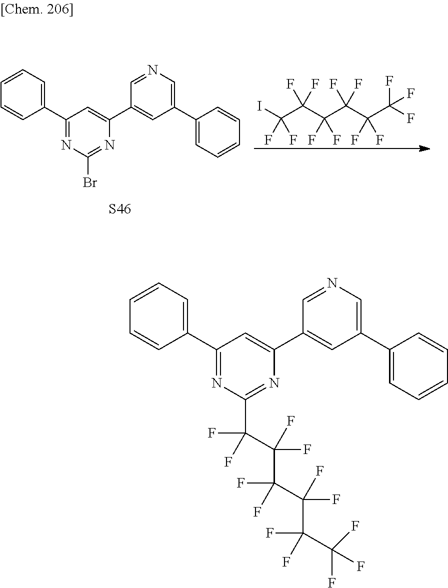

In a nitrogen atmosphere, S46 (4.0 mmol), tridecafluorohexyl iodide (12 mmol) and a copper powder (20 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z285) (yield: 51%).

The compound was identified by FDMS measurement.
FDMS: 627

### Example 91′ Evaluation of Adhesiveness of Compound (Z285) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z285) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z285) was vapor-deposited, and a 2 mm×1 mm transparent region was formed.

### Example 92 Synthesis of Compound (Z286)

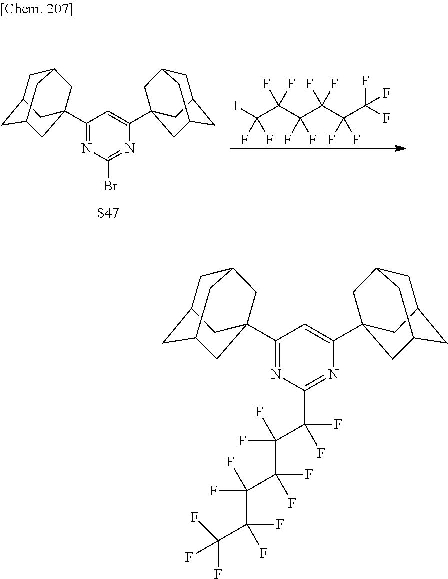

In a nitrogen atmosphere, S47 (4.0 mmol), tridecafluorohexyl iodide (12 mmol) and a copper powder (20 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z286) (yield: 68%).

The compound was identified by FDMS measurement.
FDMS: 666

### Example 92′ Evaluation of Adhesiveness of Compound (Z286) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z286) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z286) was vapor-deposited, and a 2 mm×1 mm transparent region was formed.

### Example 93 Synthesis of Compound (Z287)

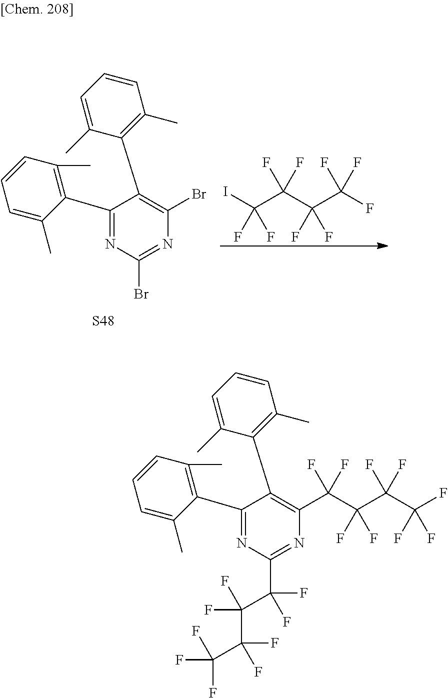

In a nitrogen atmosphere, S48 (4.0 mmol), nonafluorobutyl iodide (24 mmol) and a copper powder (40 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z287) (yield: 22%).

The compound was identified by FDMS measurement.
FDMS: 724

### Example 93′ Evaluation of Adhesiveness of Compound (Z287) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z287) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z287) was vapor-deposited, and a 2 mm×1 mm transparent region was formed.

### Example 94 Synthesis of Compound (Z288)

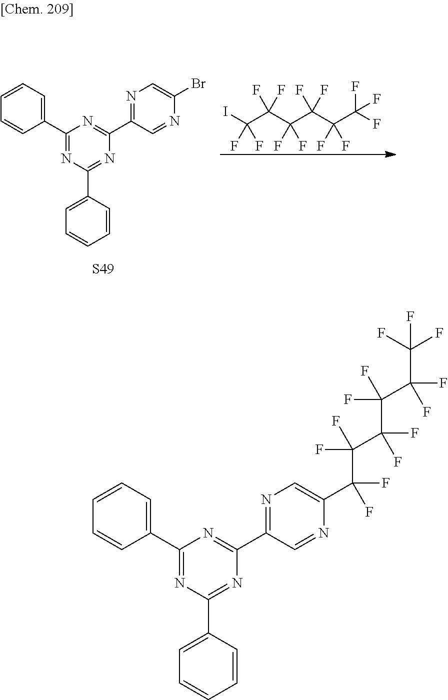 [SMILES: Brc1cnc(-c2nc(-c3ccccc3)nc(-c3ccccc3)n2)cn1 ; FC(F)(F)C(F)(F)C(F)(F)C(F)(F)C(F)(F)C(F)(F)I ; "S49" ; FC(F)(F)C(F)(F)C(F)(F)C(F)(F)C(F)(F)C(F)(F)c1cnc(-c2nc(-c3ccccc3)nc(-c3ccccc3)n2)cn1]

In a nitrogen atmosphere, S49 (4.0 mmol), tridecafluorohexyl iodide (12 mmol) and a copper powder (20 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z288) (yield: 69%).

The compound was identified by FDMS measurement.
FDMS: 629

### Example 94′ Evaluation of Adhesiveness of Compound (Z288) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z288) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z288) was vapor-deposited, and a 2 mm×1 mm transparent region was formed.

### Example 95 Synthesis of Compound (Z289)

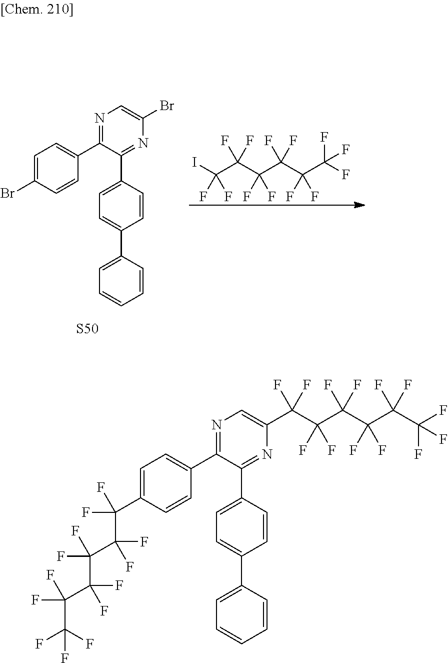 [SMILES: Brc1ccc(-c2ncc(Br)nc2-c2ccc(-c3ccccc3)cc2)cc1 ; FC(F)(F)C(F)(F)C(F)(F)C(F)(F)C(F)(F)C(F)(F)I ; "S50" ; FC(F)(F)C(F)(F)C(F)(F)C(F)(F)C(F)(F)C(F)(F)c1ccc(-c2ncc(C(F)(F)C(F)(F)C(F)(F)C(F)(F)C(F)(F)C(F)(F)F)nc2-c2ccc(-c3ccccc3)cc2)cc1]

In a nitrogen atmosphere, S50 (4.0 mmol), tridecafluorohexyl iodide (24 mmol) and a copper powder (40 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z289) (yield: 22%).

The compound was identified by FDMS measurement.
FDMS: 944

### Example 95′ Evaluation of Adhesiveness of Compound (Z289) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z289) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z289) was vapor-deposited, and a 2 mm×1 mm transparent region was formed.

### Example 96 Synthesis of Compound (Z290)

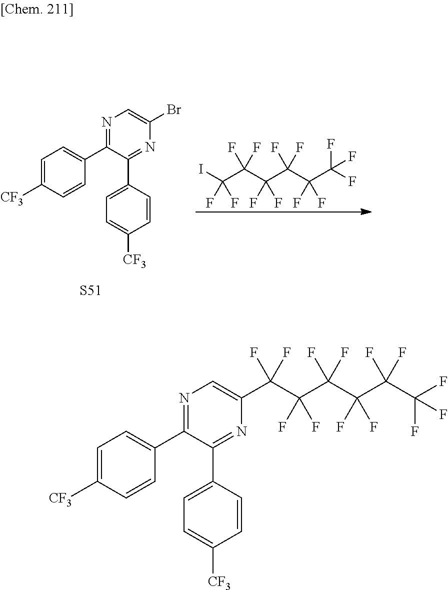 [SMILES: FC(F)(F)c1ccc(-c2ncc(Br)nc2-c2ccc(C(F)(F)F)cc2)cc1 ; FC(F)(F)C(F)(F)C(F)(F)C(F)(F)C(F)(F)C(F)(F)I ; "S51" ; FC(F)(F)c1ccc(-c2ncc(C(F)(F)C(F)(F)C(F)(F)C(F)(F)C(F)(F)C(F)(F)F)nc2-c2ccc(C(F)(F)F)cc2)cc1]

In a nitrogen atmosphere, S51 (4.0 mmol), heptadecafluorooctyl iodide (12 mmol) and a copper powder (20 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z290) (yield: 59%).

The compound was identified by FDMS measurement.
FDMS: 786

### Example 96′ Evaluation of Adhesiveness of Compound (Z290) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z290) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z290) was vapor-deposited, and a 2 mm×1 mm transparent region was formed.

### Example 97 Synthesis of Compound (Z291)

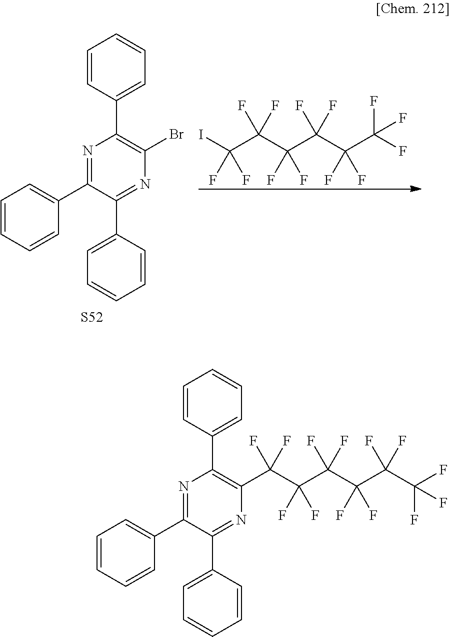

In a nitrogen atmosphere, S52 (4.0 mmol), tridecafluorohexyl iodide (12 mmol) and a copper powder (20 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z291) (yield: 31%).

The compound was identified by FDMS measurement.
FDMS: 786

### Example 97′ Evaluation of Adhesiveness of Compound (Z291) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z291) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z291) was vapor-deposited, and a 2 mm×1 mm transparent region was formed.

### Example 98 Synthesis of Compound (Z292)

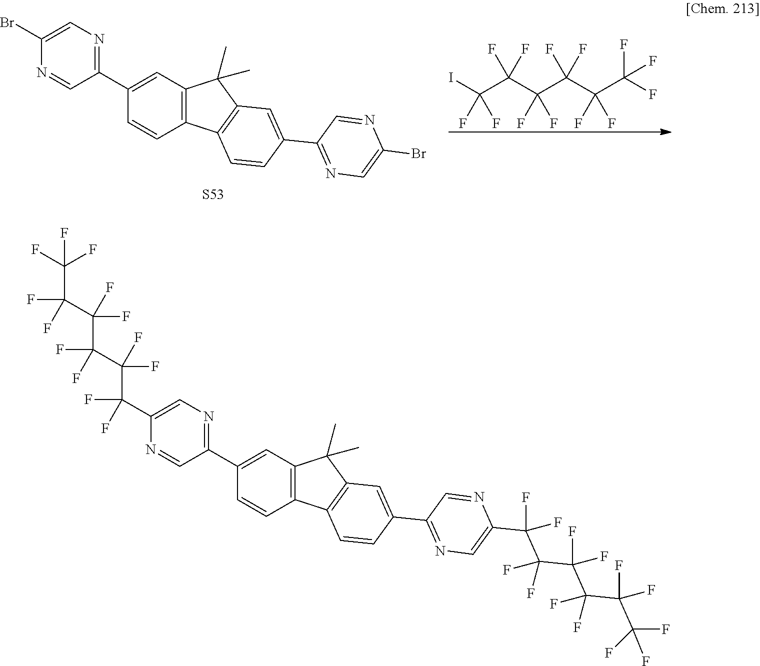 [SMILES: CC1(C)c2cc(-c3cnc(Br)cn3)ccc2-c2ccc(-c3cnc(Br)cn3)cc21 ; FC(F)(F)C(F)(F)C(F)(F)C(F)(F)C(F)(F)C(F)(F)I ; "S53" ; CC1(C)c2cc(-c3cnc(C(F)(F)C(F)(F)C(F)(F)C(F)(F)C(F)(F)C(F)(F)F)cn3)ccc2-c2ccc(-c3cnc(C(F)(F)C(F)(F)C(F)(F)C(F)(F)C(F)(F)C(F)(F)F)cn3)cc21]

In a nitrogen atmosphere, S53 (4.0 mmol), tridecafluorohexyl iodide (24 mmol) and a copper powder (40 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z292) (yield: 59%).

The compound was identified by FDMS measurement.
FDMS: 986

### Example 98′ Evaluation of Adhesiveness of Compound (Z292) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z292) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z292) was vapor-deposited, and a 2 mm×1 mm transparent region was formed.

### Example 99 Synthesis of Compound (Z293)

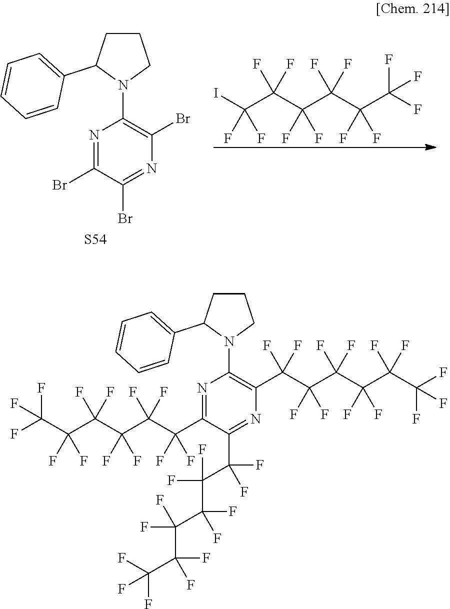 [SMILES: Brc1nc(Br)c(N2CCCC2c2ccccc2)nc1Br ; FC(F)(F)C(F)(F)C(F)(F)C(F)(F)C(F)(F)C(F)(F)I ; "S54" ; FC(F)(F)C(F)(F)C(F)(F)C(F)(F)C(F)(F)C(F)(F)c1nc(C(F)(F)C(F)(F)C(F)(F)C(F)(F)C(F)(F)C(F)(F)F)c(C(F)(F)C(F)(F)C(F)(F)C(F)(F)C(F)(F)C(F)(F)F)nc1N1CCCC1c1ccccc1]

In a nitrogen atmosphere, S54 (4.0 mmol), tridecafluorohexyl iodide (36 mmol) and a copper powder (60 mmol) were suspended in dimethyl sulfoxide (100 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z293) (yield: 15%).

The compound was identified by FDMS measurement.
FDMS: 1179

### Example 99′ Evaluation of Adhesiveness of Compound (Z293) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z293) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z293) was vapor-deposited, and a 2 mm×1 mm transparent region was formed.

### Example 100 Synthesis of Compound (Z294)

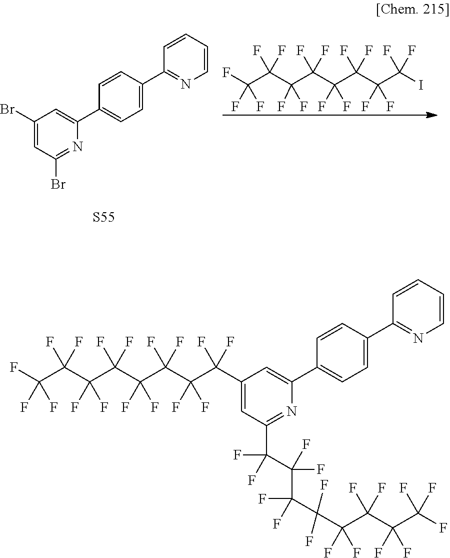

In a nitrogen atmosphere, S55 (4.0 mmol), heptadecafluorooctyl iodide (24 mmol) and a copper powder (40 mmol) were suspended in dimethyl sulfoxide (100 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z294) (yield: 69%).

The compound was identified by FDMS measurement.
FDMS: 1068

### Example 100′ Evaluation of Adhesiveness of Compound (Z294) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z294) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z294) was vapor-deposited, and a 2 mm×1 mm transparent region was formed.

### Example 101 Synthesis of Compound (Z295)

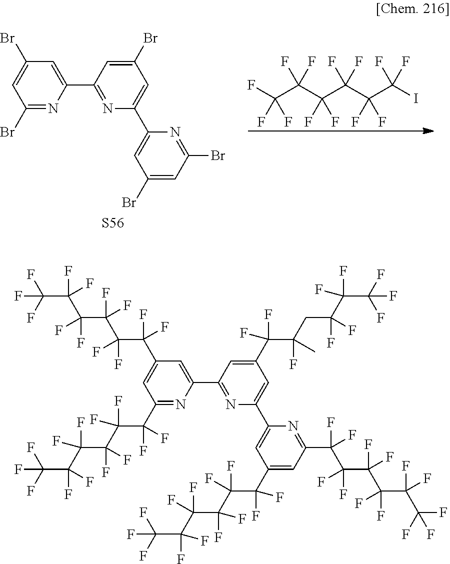 [SMILES: Brc1cc(Br)nc(-c2cc(Br)cc(-c3cc(Br)cc(Br)n3)n2)c1 ; FC(F)(F)C(F)(F)C(F)(F)C(F)(F)C(F)(F)C(F)(F)I ; "S56" ; CC(F)(CC(F)(F)C(F)(F)C(F)(F)F)C(F)(F)c1cc(-c2cc(C(F)(F)C(F)(F)C(F)(F)C(F)(F)C(F)(F)C(F)(F)F)cc(C(F)(F)C(F)(F)C(F)(F)C(F)(F)C(F)(F)C(F)(F)F)n2)nc(-c2cc(C(F)(F)C(F)(F)C(F)(F)C(F)(F)C(F)(F)C(F)(F)F)cc(C(F)(F)C(F)(F)C(F)(F)C(F)(F)C(F)(F)C(F)(F)F)n2)c1]

In a nitrogen atmosphere, S56 (4.0 mmol), tridecafluorohexyl iodide (60 mmol) and a copper powder (120 mmol) were suspended in dimethyl sulfoxide (150 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z295) (yield: 12%).

The compound was identified by FDMS measurement.
FDMS: 1822

### Example 101′ Evaluation of Adhesiveness of Compound (Z295) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z295) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z295) was vapor-deposited, and a 2 mm×1 mm transparent region was formed.

### Example 102 Synthesis of Compound (Z296)

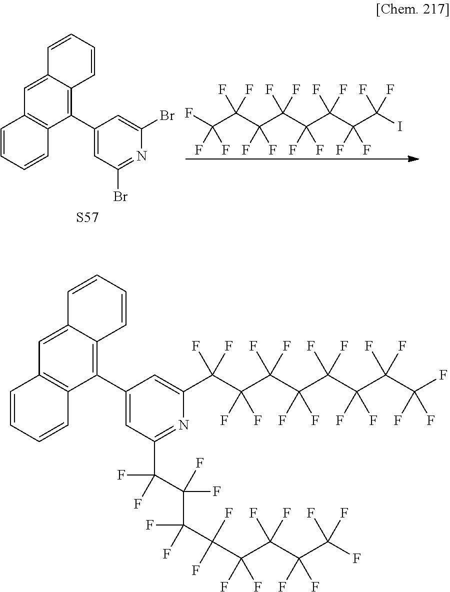

In a nitrogen atmosphere, S57 (4.0 mmol), heptadecafluorooctyl iodide (24 mmol) and a copper powder (40 mmol) were suspended in dimethyl sulfoxide (100 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z296) (yield: 68%).

The compound was identified by FDMS measurement.
FDMS: 1091

### Example 102′ Evaluation of Adhesiveness of Compound (Z296) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z296) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z296) was vapor-deposited, and a 2 mm×1 mm transparent region was formed.

### Example 103 Synthesis of Compound (Z297)

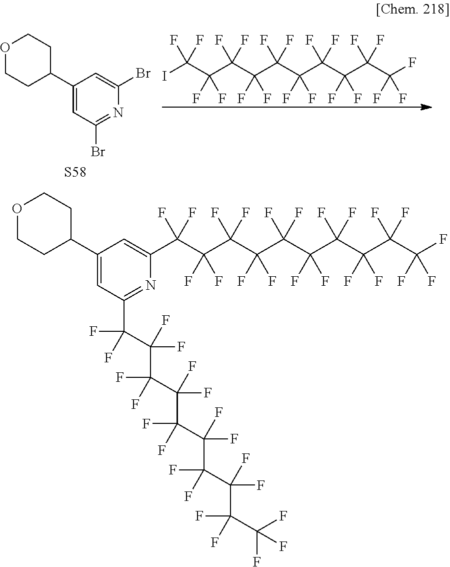

In a nitrogen atmosphere, S58 (4.0 mmol), henekicosafluorodecyl iodide (24 mmol) and a copper powder (40 mmol) were suspended in dimethyl sulfoxide (100 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z297) (yield: 22%).

The compound was identified by FDMS measurement.
FDMS: 1199

### Example 103′ Evaluation of Adhesiveness of Compound (Z297) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z297) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z297) was vapor-deposited, and a 2 mm×1 mm transparent region was formed.

### Example 104 Synthesis of Compound (Z298)

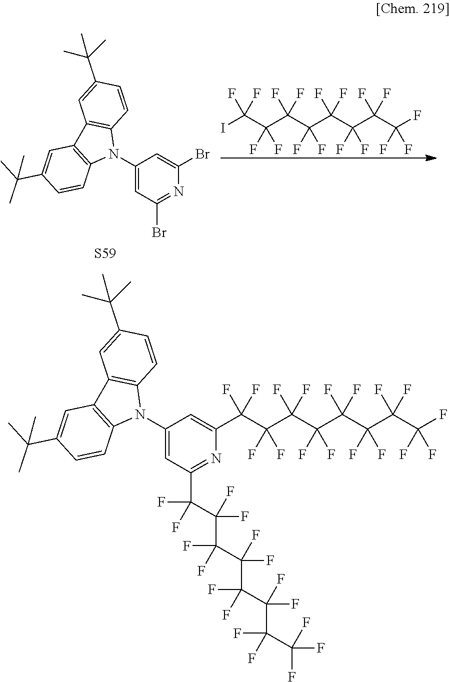

In a nitrogen atmosphere, S59 (4.0 mmol), heptadecafluorooctyl iodide (24 mmol) and a copper powder (40 mmol) were suspended in dimethyl sulfoxide (100 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z298) (yield: 22%).

The compound was identified by FDMS measurement.
FDMS: 1192

### Example 104′ Evaluation of Adhesiveness of Compound (Z298) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z298) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z298) was vapor-deposited, and a 2 mm×1 mm transparent region was formed.

### Example 105 Synthesis of Compound (Z299)

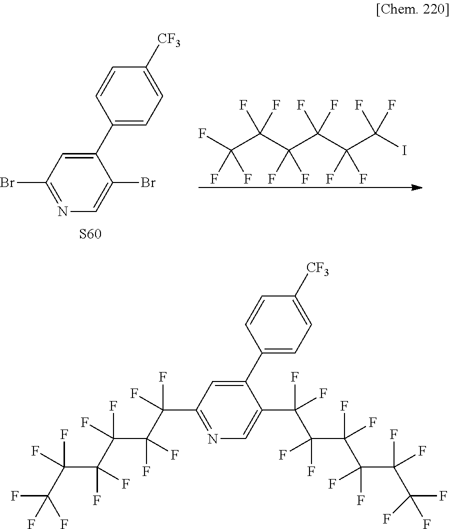 [SMILES: FC(F)(F)c1ccc(-c2cc(Br)ncc2Br)cc1 ; FC(F)(F)C(F)(F)C(F)(F)C(F)(F)C(F)(F)C(F)(F)I ; "S60" ; FC(F)(F)c1ccc(-c2cc(C(F)(F)C(F)(F)C(F)(F)C(F)(F)C(F)(F)C(F)(F)F)ncc2C(F)(F)C(F)(F)C(F)(F)C(F)(F)C(F)(F)C(F)(F)F)cc1]

In a nitrogen atmosphere, S60 (4.0 mmol), tridecafluorohexyl iodide (24 mmol) and a copper powder (40 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z299) (yield: 48%).

The compound was identified by FDMS measurement.
FDMS: 859

### Example 105′ Evaluation of Adhesiveness of Compound (Z299) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z299) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z299) was vapor-deposited, and a 2 mm×1 mm transparent region was formed.

### Example 106 Synthesis of Compound (Z300)

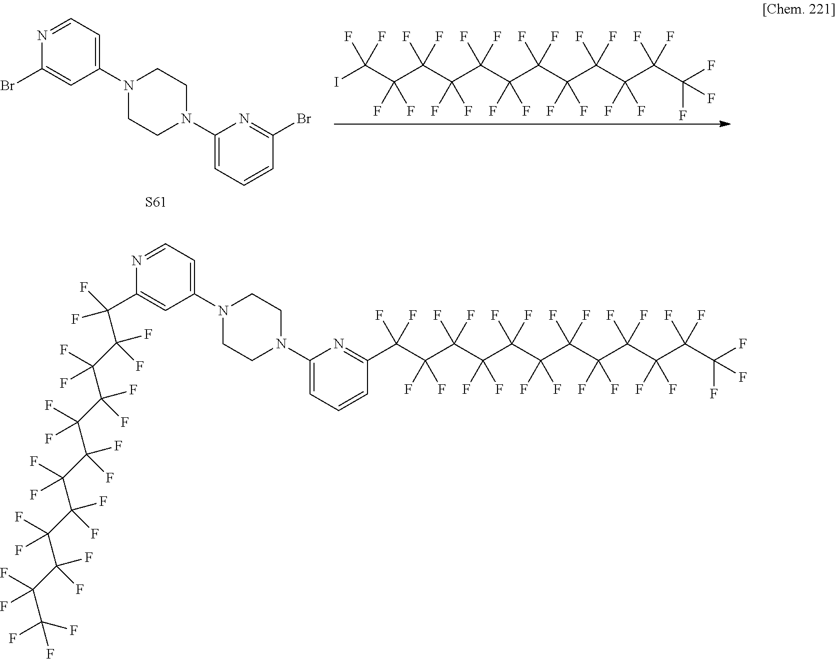 [SMILES: Brc1cc(N2CCN(c3cccc(Br)n3)CC2)ccn1 ; FC(F)(F)C(F)(F)C(F)(F)C(F)(F)C(F)(F)C(F)(F)C(F)(F)C(F)(F)C(F)(F)C(F)(F)C(F)(F)C(F)(F)I ; "S61" ; FC(F)(F)C(F)(F)C(F)(F)C(F)(F)C(F)(F)C(F)(F)C(F)(F)C(F)(F)C(F)(F)C(F)(F)C(F)(F)C(F)(F)c1cc(N2CCN(c3cccc(C(F)(F)C(F)(F)C(F)(F)C(F)(F)C(F)(F)C(F)(F)C(F)(F)C(F)(F)C(F)(F)C(F)(F)C(F)(F)C(F)(F)F)n3)CC2)ccn1]

In a nitrogen atmosphere, S61 (4.0 mmol), perfluorododecyl iodide (24 mmol) and a copper powder (40 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z300) (yield: 20%).

The compound was identified by FDMS measurement.
FDMS: 1476

### Example 106′ (Evaluation of Adhesiveness of Compound (Z300) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z300) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z300) was vapor-deposited, and a 2 mm×1 mm transparent region was formed.

### Example 107 Synthesis of Compound (Z301)

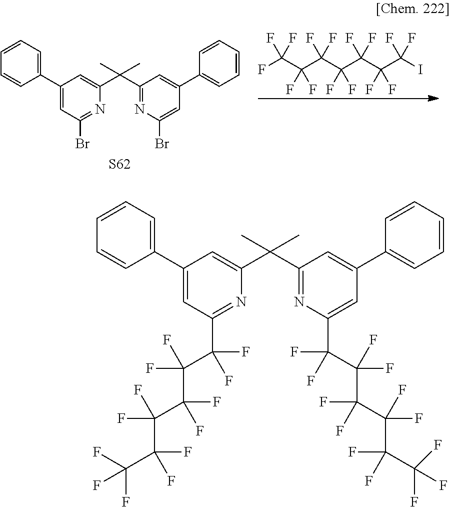

In a nitrogen atmosphere, S62 (4.0 mmol), tridecafluorohexyl iodide (24 mmol) and a copper powder (40 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z301) (yield: 48%).

The compound was identified by FDMS measurement.
FDMS: 986

### Example 107′ Evaluation of Adhesiveness of Compound (Z301) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z301) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z301) was vapor-deposited, and a 2 mm×1 mm transparent region was formed.

### Example 108 Synthesis of Compound (Z302)

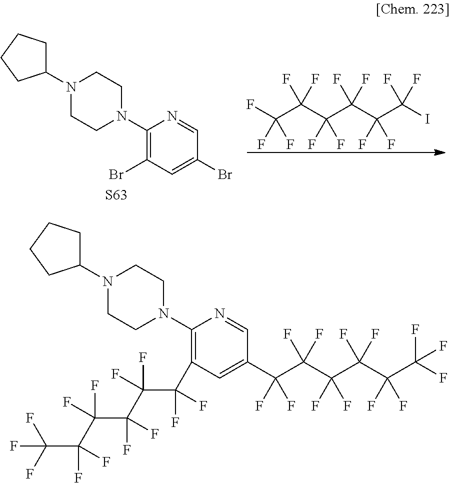 [SMILES: Brc1cnc(N2CCN(C3CCCC3)CC2)c(Br)c1 ; FC(F)(F)C(F)(F)C(F)(F)C(F)(F)C(F)(F)C(F)(F)I ; "S63" ; FC(F)(F)C(F)(F)C(F)(F)C(F)(F)C(F)(F)C(F)(F)c1cnc(N2CCN(C3CCCC3)CC2)c(C(F)(F)C(F)(F)C(F)(F)C(F)(F)C(F)(F)C(F)(F)F)c1]

In a nitrogen atmosphere, S63 (4.0 mmol), tridecafluorohexyl iodide (24 mmol) and a copper powder (40 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z302) (yield: 51%).

The compound was identified by FDMS measurement.
FDMS: 867

### Example 108′ Evaluation of Adhesiveness of Compound (Z302) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z302) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z302) was vapor-deposited, and a 2 mm×1 mm transparent region was formed.

### Example 109 Synthesis of Compound (Z555)

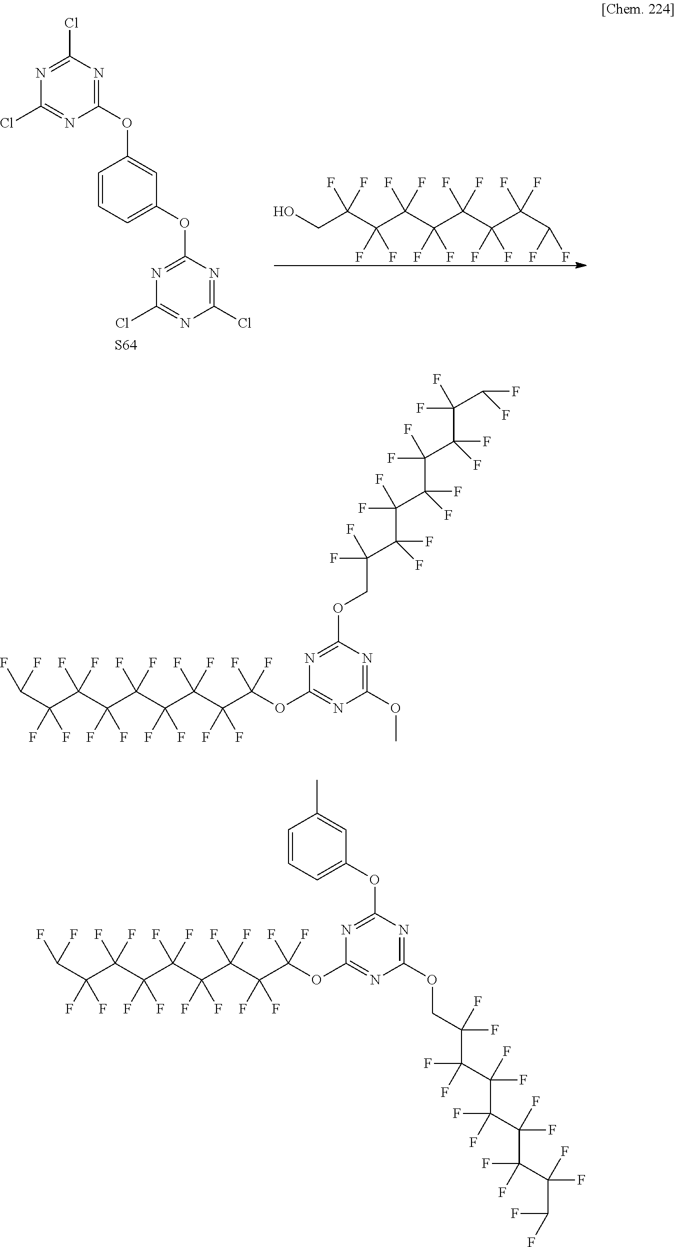 [SMILES: Clc1nc(Cl)nc(Oc2cccc(Oc3nc(Cl)nc(Cl)n3)c2)n1 ; OCC(F)(F)C(F)(F)C(F)(F)C(F)(F)C(F)(F)C(F)(F)C(F)(F)C(F)F ; "S64" ; COc1nc(OCC(F)(F)C(F)(F)C(F)(F)C(F)(F)C(F)(F)C(F)(F)C(F)(F)C(F)F)nc(OC(F)(F)C(F)(F)C(F)(F)C(F)(F)C(F)(F)C(F)(F)C(F)(F)C(F)(F)C(F)F)n1 ; Cc1cccc(Oc2nc(OCC(F)(F)C(F)(F)C(F)(F)C(F)(F)C(F)(F)C(F)(F)C(F)(F)C(F)F)nc(OC(F)(F)C(F)(F)C(F)(F)C(F)(F)C(F)(F)C(F)(F)C(F)(F)C(F)(F)C(F)F)n2)c1]

In a nitrogen atmosphere, sodium hydride (24.0 mmol) was suspended in tetrahydrofuran (20 ml) and was stirred at 0° C., and 1H, 1H, 9H-hexadecafluoro-1-nonanol (22.0 mmol) was added dropwise to the suspension over 20 minutes. After completion of the dropwise addition, the temperature was returned to room temperature, and S64 (5.0 mmol) dissolved in tetrahydrofuran (50 ml) was added dropwise over 20 minutes. The dropwise addition was followed by stirring at 60° C. for 5 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (Z555) (yield: 56%).

The compound was identified by FDMS measurement.
FDMS: 1988

### Example 109′ Evaluation of Adhesiveness of Compound (Z555) to Metal

A glass substrate was subjected to boiling cleaning (boiling cleaning with isopropyl alcohol), was subjected to ultraviolet-ozone cleaning, and was then placed in a vacuum evaporator, which was evacuated with a vacuum pump to 1.0×10-4 Pa or less. A 20-nm film of the compound (Z555) was formed at a vapor-deposition rate of 0.2 nm/s on a glass substrate on which a metal mask with a 2 mm×1 mm opening was disposed. The metal mask was then removed, and 10 nm of silver was vapor-deposited at a vapor-deposition rate of 0.2 nm/s. A film of silver was not formed in a portion where the compound (Z555) was vapor-deposited, and a 2 mm×1 mm transparent region was formed.

### Comparative Example 1′ (Evaluation of Adhesiveness of Compound (X1) to Metal)

The following compound (X1) was used as a material for metal patterning to evaluate metal adhesion.

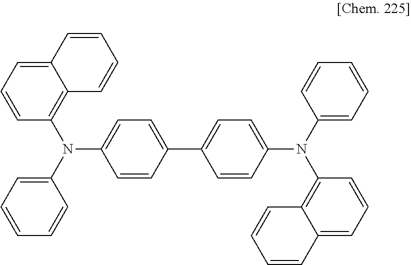 [SMILES: c1ccc(N(c2ccc(-c3ccc(N(c4ccccc4)c4cccc5ccccc45)cc3)cc2)c2cccc3ccccc23)cc1]

The adhesiveness of a metal to the compound (X1) was evaluated in the same manner as in Example 1′. A silver film was also formed on a film of the compound (X1), and a transparent region was not formed.

### Comparative Example 2′ (Evaluation of Adhesiveness of Compound (X2) to Metal)

The following compound (X2) was used as a material for metal patterning to evaluate metal adhesion.

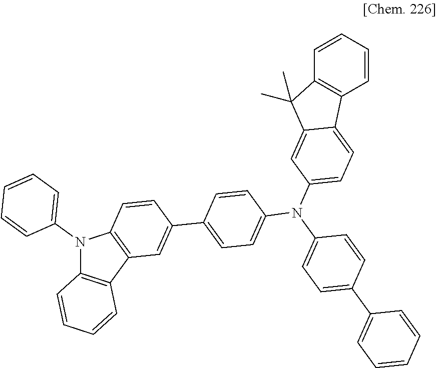 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

The adhesiveness of a metal to the compound (X2) was evaluated in the same manner as in Example 1′. A silver film was also formed on a film of the compound (X2), and a transparent region was not formed.

### Comparative Example 3′ (Evaluation of Adhesiveness of Compound (X3) to Metal)

The following compound (X3) was used as a material for metal patterning to evaluate metal adhesion.

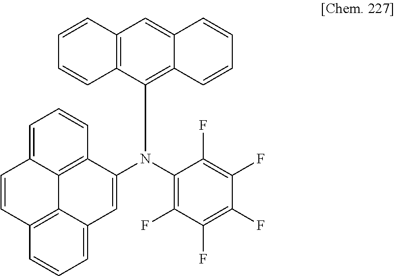 [SMILES: Fc1c(F)c(F)c(N(c2c3ccccc3cc3ccccc23)c2cc3cccc4ccc5cccc2c5c43)c(F)c1F]

The adhesiveness of a metal to the compound (X3) was evaluated in the same manner as in Example 1′. A silver film was also formed on a film of the compound (X3), and a transparent region was not formed.

The following compound (X4) was used as a material for metal patterning to evaluate metal adhesion.

### Comparative Example 4′ (Evaluation of Adhesiveness of Compound (X4) to Metal)

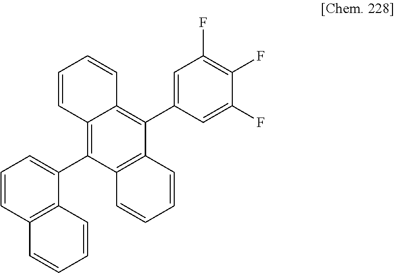 [SMILES: Fc1cc(-c2c3ccccc3c(-c3cccc4ccccc34)c3ccccc23)cc(F)c1F]

The adhesiveness of a metal to the compound (X4) was evaluated in the same manner as in Example 1′. A silver film was also formed on a film of the compound (X4), and a transparent region was not formed.

The following compound (X5) was used as a material for metal patterning to evaluate metal adhesion.

### Comparative Example 5′ (Evaluation of Adhesiveness of Compound (X5) to Metal)

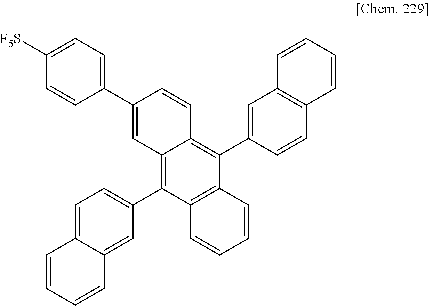

The adhesiveness of a metal to the compound (X5) was evaluated in the same manner as in Example 1′. A silver film was also formed on a film of the compound (X5), and a transparent region was not formed.

### Comparative Example 6′ Synthesis of Compound (X6)

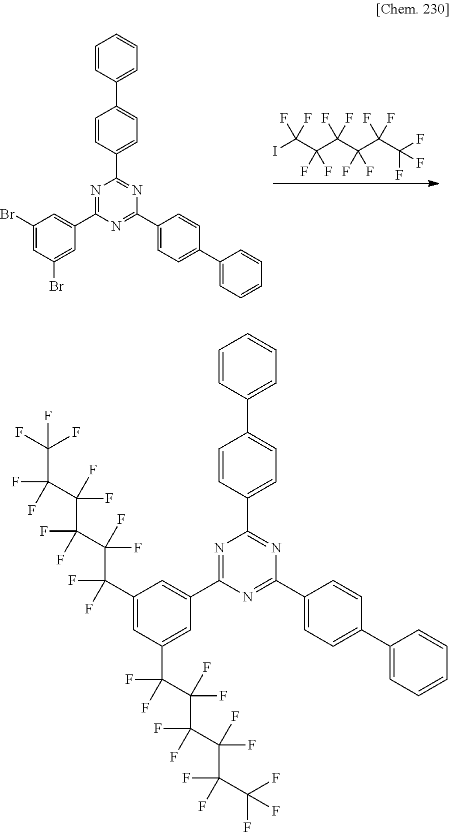

In a nitrogen atmosphere, 2,4-bis([1,1′-biphenyl]-4-yl)-6-(3,5-dibromophenyl)-1,3,5-triazine (4.0 mmol), tridecafluorohexyl iodide (24 mmol) and a copper powder (40 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (X6) (yield: 35%).

The compound was identified by FDMS measurement.
FDMS: 1097

### Comparative Example 6′ (Evaluation of Adhesiveness of Compound (X6) to Metal)

The adhesiveness of a metal to the compound (X6) was evaluated in the same manner as in Example 1′. A silver film was also formed on a film of the compound (X6), and a transparent region was not formed.

### Comparative Example 7′ Synthesis of Compound (X7)

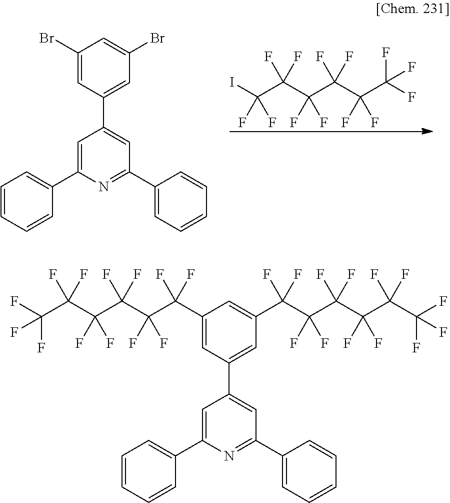 [SMILES: Brc1cc(Br)cc(-c2cc(-c3ccccc3)nc(-c3ccccc3)c2)c1 ; FC(F)(F)C(F)(F)C(F)(F)C(F)(F)C(F)(F)C(F)(F)I ; FC(F)(F)C(F)(F)C(F)(F)C(F)(F)C(F)(F)C(F)(F)c1cc(-c2cc(-c3ccccc3)nc(-c3ccccc3)c2)cc(C(F)(F)C(F)(F)C(F)(F)C(F)(F)C(F)(F)C(F)(F)F)c1]

In a nitrogen atmosphere, 4-(3,5-dibromophenyl)-2,6-diphenylpyridine (4.0 mmol), tridecafluorohexyl iodide (24 mmol) and a copper powder (40 mmol) were suspended in dimethyl sulfoxide (50 mL) and were stirred at 140° C. for 24 hours. After cooling to room temperature, the suspension was separated using pure water and chloroform, and the organic layer was washed with saturated aqueous sodium chloride. The organic layer was dried over anhydrous magnesium sulfate and was then purified by silica gel column chromatography to produce a white solid target compound (X7) (yield: 48%).

The compound was identified by FDMS measurement.
FDMS: 943

### Comparative Example 7′ (Evaluation of Adhesiveness of Compound (X7) to Metal)

The adhesiveness of a metal to the compound (X7) was evaluated in the same manner as in Example 1′. A silver film was also formed on a film of the compound (X7), and a transparent region was not formed.

### Comparative Example 8′ (Evaluation of Adhesiveness of Compound (X8) to Metal)

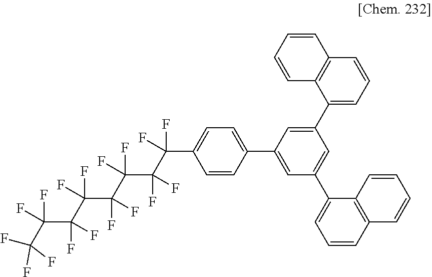 [SMILES: FC(F)(F)C(F)(F)C(F)(F)C(F)(F)C(F)(F)C(F)(F)C(F)(F)C(F)(F)c1ccc(-c2cc(-c3cccc4ccccc34)cc(-c3cccc4ccccc34)c2)cc1]

The adhesiveness of a metal to the compound (X8) was evaluated in the same manner as in Example 1′. A silver film was also formed on a film of the compound (X8), and a transparent region was not formed.

### <<Measurement of Transmittance>>

Metal adhesion can be evaluated by measuring transmittance, and metal adhesion reduces transmittance. The transmittance decreases with increasing adhesion amount. Example 1″ Measurement of Transmittance of Light through Compound (Z1)

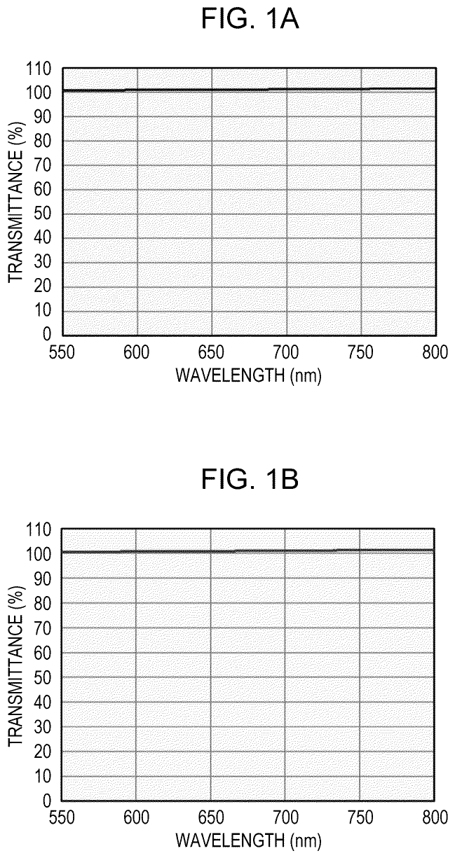

A glass substrate was subjected to boiling cleaning with isopropyl alcohol, was subjected to ultraviolet-ozone cleaning, and was then placed in a vacuum evaporator, which was evacuated with a vacuum pump to 1.0×10⁻⁴ Pa or less. A 20-nm film of the compound (Z1) as a material for metal patterning was formed on a glass substrate at a vapor-deposition rate of 0.1 nm/s. FIG. 1A shows transmittance measurement results. Furthermore, 10 nm of silver was vapor-deposited on the thin film of the compound (Z1) at a vapor-deposition rate of 0.1 nm/s. FIG. 1B shows transmittance measurement results. In FIG. 1B, the transmittance in the range of 550 to 800 nm after metal evaporation was “80% or more”, and the formation of a metal film was suppressed.

### Example 8″ Measurement of Transmittance of Light Through Compound (Z190)

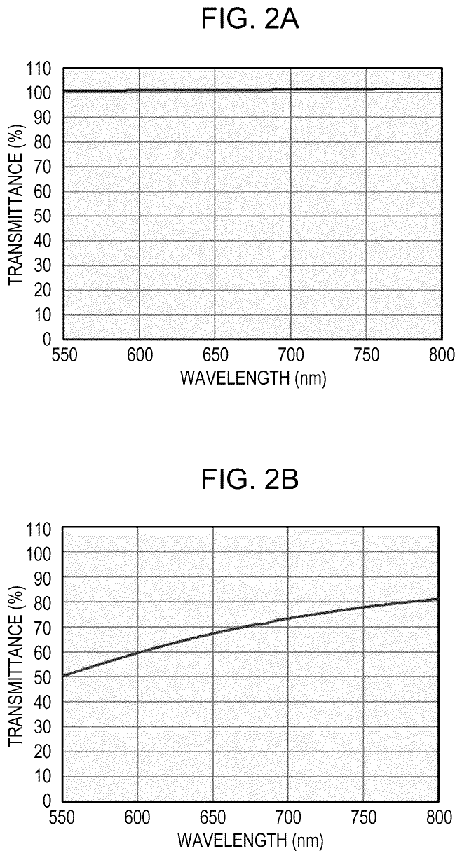

The transmittance of light through the compound (Z190) was measured in the same manner as in Example 1″. FIGS. 2A and 2B show transmittance measurement results before metal evaporation and after metal evaporation, respectively.

In FIG. 2B, the transmittance in the range of 550 to 800 nm after metal evaporation was “50% or more”, and the formation of a metal film was suppressed.

### Comparative Example 1″ Measurement of Transmittance of Light Through Compound (X1)

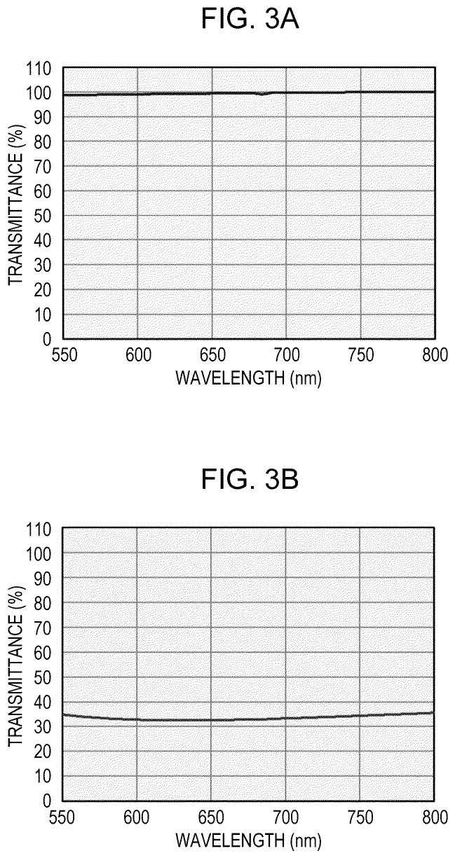

The transmittance of light through the compound (X1) was measured in the same manner as in Example 1″. FIGS. 3A and 3B show transmittance measurement results before metal evaporation and after metal evaporation, respectively.

In FIG. 3B, the transmittance in the range of 550 to 800 nm after metal evaporation was “30% or more”, and the formation of a metal film could not be suppressed.

### Comparative Example 1-4″ Measurement of Transmittance of Light through Compound (X6)

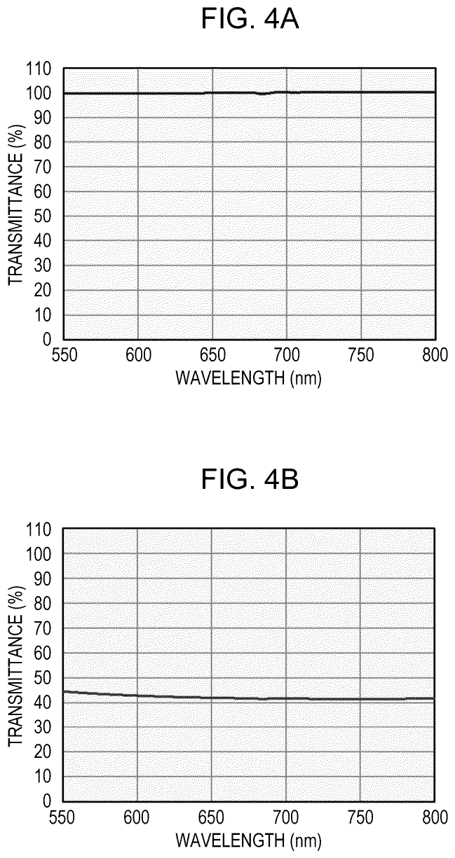

The transmittance of light through the compound (X6) was measured in the same manner as in Example 1″. FIGS. 4A and 4B show transmittance measurement results before metal evaporation and after metal evaporation, respectively.

In FIG. 4B, the transmittance in the range of 550 to 800 nm after metal evaporation was “40% or more”, and the formation of a metal film could not be suppressed.

The transmittance of light through each of the compounds Z174, Z177, Z180, Z189, Z190, Z204, Z205, Z206, Z207, Z228, Z245, Z256, Z275, X1, X2, X3, X4, X5, X6, X7, and X8 was measured in the same manner as in Example 1″. Table 1 shows the results.

TABLE 1

| | | Transmittance after metal |
|---|---|---|
| | Compound No. | evaporation |
| Example 1″ | Z1 | ≥80% |
| Example 2″ | Z174 | ≥80% |
| Example 3″ | Z177 | ≥80% |
| Example 4″ | Z180 | ≥80% |
| Example 7″ | Z189 | ≥50% |
| Example 8″ | Z190 | ≥50% |
| Example 10″ | Z204 | ≥80% |
| Example 11″ | Z205 | ≥80% |
| Example 12″ | Z206 | ≥80% |
| Example 13″ | Z207 | ≥80% |
| Example 34″ | Z228 | ≥80% |
| Example 51″ | Z245 | ≥80% |
| Example 62″ | Z256 | ≥50% |
| Example 81″ | Z275 | ≥80% |
| Comparative Example 1″ | X1 | ≥30% |
| Comparative Example 2″ | X2 | ≥30% |
| Comparative Example 3″ | X3 | ≥30% |
| Comparative Example 4″ | X4 | ≥30% |
| Comparative Example 5″ | X5 | ≥30% |
| Comparative Example 6″ | X6 | ≥40% |
| Comparative Example 7″ | X7 | ≥40% |
| Comparative Example 8″ | X8 | ≥40% |

While the present invention has been described in detail with reference to specific embodiments, it is apparent to a person skilled in the art that various alterations and modifications may be made to the embodiments without departing from the essence and scope of the present invention.

The entire contents of the description, claims, drawings and abstract of Japanese Patent Application No. 2022-018503 filed Feb. 9, 2022, Japanese Patent Application No. 2022-192376 filed Nov. 30, 2022, and Japanese Patent Application No. 2023-018054 filed Feb. 9, 2023 are incorporated herein by reference as the disclosure of the description of the present invention.

1. A material for metal patterning represented by the formula (A1) or (B1): 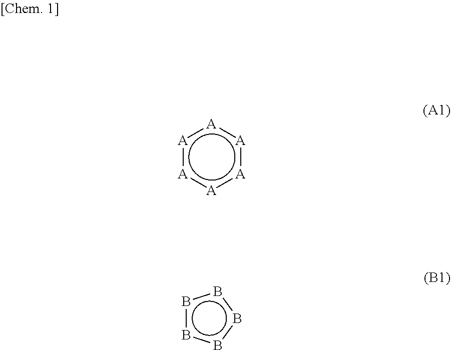
wherein
A each independently denotes N or CR, at least one thereof being N and at least one thereof being CR;
B each independently denotes N, S, O or CR, at least one thereof being N, S or O and at least one thereof being CR;
R each independently denotes the formula (A01) or the formula (A02), at least one thereof being the formula (A01): 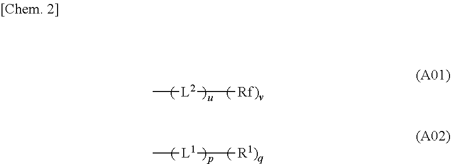
L¹ and L² each independently denote
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR²;
Rf each independently denotes
a monovalent or divalent substituent with 1 or more carbon atoms containing 3 or more fluorine atoms;
R¹ and R² each independently denote
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent or divalent heteroaliphatic hydrocarbon group with 3 to 18 carbon atoms, or
H, D (deuterium atom) or F (fluorine atom);
p each independently denotes an integer in the range of 0 to 12,
q each independently denotes an integer in the range of 1 to 6,
u each independently denotes an integer in the range of 0 to 12, and
v each independently denotes an integer in the range of 1 to 6.

2. The material for metal patterning according to claim 1, wherein Rf is represented by the following formula (001): 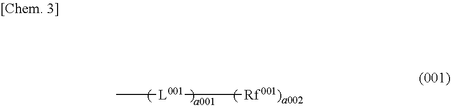
wherein
Rf⁰⁰¹ each independently denotes
a linear, branched or cyclic aliphatic hydrocarbon group with 1 to 18 carbon atoms containing 3 or more fluorine atoms;
L⁰⁰¹ each independently denotes
a linear, branched or cyclic divalent aliphatic hydrocarbon group with 1 to 18 carbon atoms optionally substituted with O, S or a fluorine atom, or
a vinylene group that may be substituted with fluorine and may form a ring;
a001 each independently denotes an integer in the range of 0 to 4; and
a002 each independently denotes an integer in the range of 1 to 4.

3. The material for metal patterning according to claim 1, wherein u in at least one formula (A01) in the formula (A1) is 0, and u in at least one formula (A01) in the formula (B1) is 0.

4. The material for metal patterning according to claim 1, represented by any one of the formulae (101) to (105): 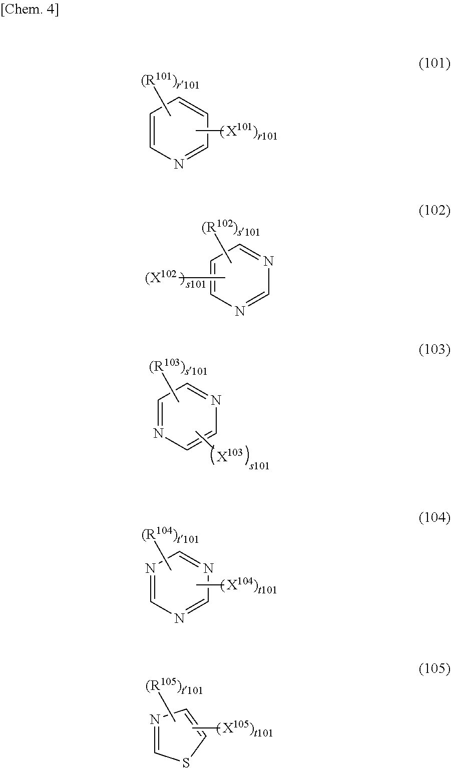
wherein
X¹⁰¹, X¹⁰², X¹⁰³, X¹⁰⁴ and X¹⁰⁵ are each independently represented by the formula (111): 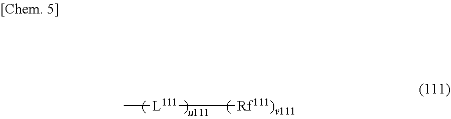
wherein
R¹⁰¹, R¹⁰², R¹⁰³, R¹⁰⁴ and R¹⁰⁵ are each independently represented by the formula (121): 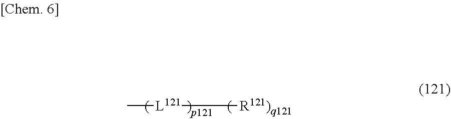
L¹¹¹ and L¹²¹ each independently denote
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted linear, branched or cyclic divalent to tetravalent aliphatic hydrocarbon group with 1 to 18 carbon atoms,
an optionally substituted cyclic divalent to tetravalent heteroaliphatic hydrocarbon group with 3 to 18 carbon atoms, or
O, S, Si or NR¹¹¹;
Rf¹¹¹ each independently denotes a structure represented by the formula (001);
R¹¹¹ and R¹²¹ each independently denote
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 3 to 18 carbon atoms, or H, D (deuterium atom) or F (fluorine atom);
r101 denotes an integer in the range of 1 to 5, and r′101 denotes an integer in the range of 0 to 4, provided that (r101+r′101) is an integer of 5 or less,
s¹⁰¹ each independently denotes an integer in the range of 1 to 4, and s′¹⁰¹ each independently denotes an integer in the range of 0 to 3, provided that (s¹⁰¹+s′¹⁰¹) is an integer of 4 or less,
t101 each independently denotes an integer in the range of 1 to 3, and t′101 denotes an integer in the range of 0 to 2, provided that (t101+t′101) is an integer of 3 or less,
u111 each independently denotes an integer in the range of 0 to 12,
v111 each independently denotes an integer in the range of 1 to 6,
p121 each independently denotes an integer in the range of 0 to 12, and
q121 each independently denotes an integer in the range of 1 to 6,
provided that u111 in at least one X¹⁰¹, at least one X¹⁰², at least one X¹⁰³, at least one X¹⁰⁴ and at least one X¹⁰⁵ each independently denotes 0.

5. The material for metal patterning according to claim 4, wherein
when L¹¹¹ and L¹²¹ denote a substituted aromatic hydrocarbon group, a substituted heteroaromatic group, a substituted aliphatic hydrocarbon group or a substituted heteroaliphatic hydrocarbon group, these groups are each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a cyano group, a fluorine atom, a deuterium atom and a structure represented by the formula (001), and
a combination of these groups.

6. The material for metal patterning according to claim 1, wherein
when R¹ and R² denote a substituted aromatic hydrocarbon group, a substituted heteroaromatic group, a substituted aliphatic hydrocarbon group or a substituted heteroaliphatic hydrocarbon group, these groups are each independently substituted with one or more groups selected from the group consisting of
a linear, branched or cyclic aliphatic hydrocarbon group with 1 to 18 carbon atoms,
a linear, branched or cyclic alkoxy group with 1 to 18 carbon atoms,
an aromatic hydrocarbon group with 6 to 20 carbon atoms,
a heteroaromatic group with 3 to 20 carbon atoms,
a hydrogen atom, a fluorine atom, a deuterium atom and a structure represented by the formula (001), and
a combination of these groups.

7. The material for metal patterning according to claim 4, wherein a ratio of the number of fluorine atoms to the number of carbon atoms in at least one Rf¹¹¹ is 50% or more.

8. The material for metal patterning according to claim 4, wherein R¹⁰¹, R¹⁰², R¹⁰³, R¹⁰⁴ and R¹⁰⁵ are each independently represented by the formula (131): 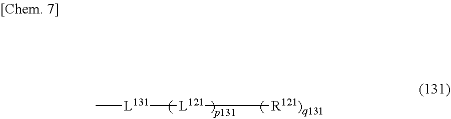
L¹²¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O, S or NR¹¹¹;
L¹³¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O or S;
R¹¹¹ and R¹²¹ each independently denote an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted linear, branched or cyclic monovalent aliphatic hydrocarbon group with 1 to 18 carbon atoms,
an optionally substituted cyclic monovalent heteroaliphatic hydrocarbon group with 3 to 18 carbon atoms, or
H, D (deuterium atom) or F (fluorine atom);
p131 each independently denotes an integer in the range of 0 to 11, and
q131 each independently denotes an integer in the range of 1 to 6.

9. The material for metal patterning according to claim 8, wherein the monocyclic, linked or condensed divalent to tetravalent aromatic hydrocarbon group represented by L¹²¹ or L¹³¹ is phenyl or has a structure in which a plurality of benzene rings are linked or condensed.

10. The material for metal patterning according to claim 8, wherein the monocyclic, linked or condensed divalent to tetravalent heteroaromatic group represented by L¹²¹ or L¹³¹ has N, O or S as a heteroatom and is a 5-membered ring, a 6-membered ring or a condensed structure thereof.

11. The material for metal patterning according to claim 8, wherein
the monocyclic, linked or condensed divalent to tetravalent aromatic hydrocarbon group represented by L¹²¹ or L¹³¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene or dibenzochrysene,
the monocyclic, linked or condensed divalent to tetravalent heteroaromatic group is pyridine, pyrimidine, pyrazine, triazine, carbazole, furan, thiophene, benzofuran, benzothiophene, benzodioxin, dibenzofuran, dibenzothiophene, thiazole, dibenzodioxin or benzothiazole,
the cyclic divalent to tetravalent aliphatic hydrocarbon group is adamantane, diamantane or cyclohexane, and
the cyclic divalent to tetravalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

12. The material for metal patterning according to claim 8, wherein
a substituent of L¹²¹ and L¹³¹ is each independently one or more groups selected from the group consisting of
a methyl group, an ethyl group, a methoxy group, an ethoxy group, a trifluoromethyl group, a trifluoromethoxy group, a perfluoroalkyl group with 2 to 10 carbon atoms, a perfluoroalkoxy group with 2 to 10 carbon atoms, a cyano group, a deuterium atom, a fluorine atom, a phenyl group, a biphenylyl group, a naphthyl group, a phenanthryl group, a pyridyl group, a carbazolyl group, a dibenzothienyl group, a dibenzofuranyl group and a structure represented by the formula (001), and
a combination of these groups.

13. The material for metal patterning according to claim 3, wherein the monocyclic, linked or condensed divalent to tetravalent aromatic hydrocarbon group represented by L¹¹¹ is a phenyl group or has a structure in which a plurality of benzene rings are linked or condensed.

14. The material for metal patterning according to claim 3, wherein the monocyclic, linked or condensed divalent to tetravalent heteroaromatic group represented by L¹¹¹ has N, O or S as a heteroatom and is a 5-membered ring, a 6-membered ring or a condensed structure thereof.

15. The material for metal patterning according to claim 3, wherein
the monocyclic, linked or condensed divalent to tetravalent aromatic hydrocarbon group represented by L¹¹¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene or dibenzochrysene,
the monocyclic, linked or condensed divalent to tetravalent heteroaromatic group is pyridine, pyrimidine, pyrazine, triazine, carbazole, furan, thiophene, benzofuran, benzothiophene, benzodioxin, dibenzofuran, dibenzothiophene, thiazole, dibenzodioxin or benzothiazole,
the cyclic divalent to tetravalent aliphatic hydrocarbon group is adamantane, diamantane or cyclohexane, and
the cyclic divalent to tetravalent heteroaliphatic hydrocarbon group is morpholine, piperazine, homopiperazine, hexahydro-1,3,5-triazine, 1,4-dioxin, 1,4-dithiane, 4,4′-bipiperidine, diazabicyclo[2,2,2]octane, octahydro-1H-pyrrolo[3,4-b]pyridine or 1,4,7,10-tetraazacyclododecane.

16. The material for metal patterning according to claim 3, wherein
a substituent of L¹¹¹ is each independently one or more groups selected from the group consisting of
a methyl group, an ethyl group, a methoxy group, an ethoxy group, a trifluoromethyl group, a trifluoromethoxy group, a perfluoroalkyl group with 2 to 10 carbon atoms, a perfluoroalkoxy group with 2 to 10 carbon atoms, a cyano group, a deuterium atom, a fluorine atom, a phenyl group, a biphenylyl group, a naphthyl group, a phenanthryl group, a pyridyl group, a carbazolyl group, a dibenzothienyl group, a dibenzofuranyl group and a structure represented by the formula (001), and
a combination of these groups.

17. The material for metal patterning according to claim 8, wherein the monocyclic, linked or condensed monovalent aromatic hydrocarbon group represented by R¹¹¹ or R¹²¹ is a phenyl group or has a structure in which a plurality of benzene rings are linked or condensed.

18. The material for metal patterning according to claim 4, wherein the monocyclic, linked or condensed monovalent heteroaromatic group represented by R¹¹¹ or R¹²¹ has N, O or S as a heteroatom and is a 5-membered ring, a 6-membered ring or a condensed structure thereof.

19. The material for metal patterning according to claim 4, wherein
the monocyclic, linked or condensed monovalent aromatic hydrocarbon group represented by R¹¹¹ or R¹²¹ is phenyl, biphenyl, terphenyl, naphthalene, fluorene, spirobifluorene, 9,9-dimethylfluorene, 9,9-diphenylfluorene, benzofluorene, phenanthrene, fluoranthene, triphenylene, anthracene, pyrene, chrysene, perylene, benzochrysene or dibenzochrysene,
the monocyclic, linked or condensed monovalent heteroaromatic group is pyridine, pyrimidine, pyrazine, triazine, carbazole, furan, thiophene, benzofuran, benzothiophene, benzodioxin, dibenzofuran, dibenzothiophene, dibenzodioxin, thiazole or benzothiazole,
the linear, branched or cyclic monovalent aliphatic hydrocarbon group is adamantane, diamantane, cyclohexane or methane, and
the cyclic monovalent heteroaliphatic hydrocarbon group is piperazine, homopiperazine, hexahydro-1,3,5-triazine or 1,4-dioxin.

20. The material for metal patterning according to claim 4, wherein
a substituent of R¹¹¹ and R¹²¹ is each independently one or more groups selected from the group consisting of
a methyl group, an ethyl group, a methoxy group, an ethoxy group, a trifluoromethyl group, a trifluoromethoxy group, a perfluoroalkyl group with 2 to 10 carbon atoms, a perfluoroalkoxy group with 2 to 10 carbon atoms, a cyano group, a deuterium atom, a fluorine atom, a phenyl group, a biphenylyl group, a naphthyl group, a phenanthryl group, a pyridyl group, a carbazolyl group, a dibenzothienyl group, a dibenzofuranyl group and a structure represented by the formula (001), and
a combination of these groups.

21. A heterocyclic compound represented by the formula (301) or (302): 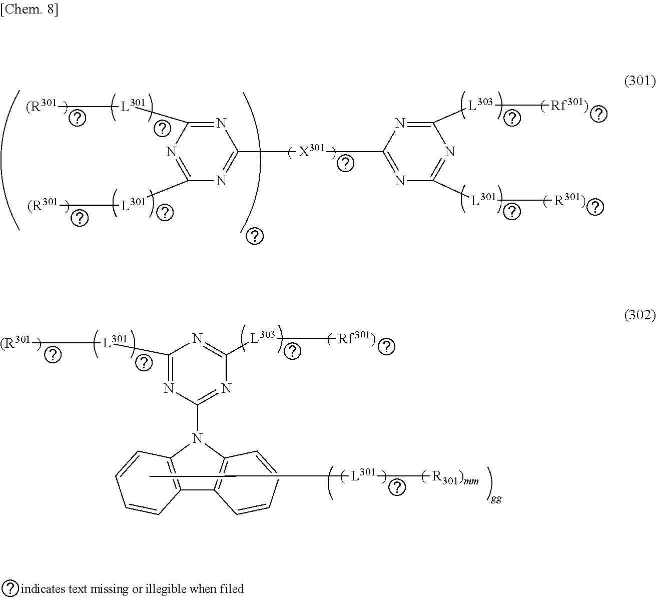
L³⁰¹ each independently denotes
an optionally substituted monocyclic, linked or condensed divalent to tetravalent aromatic hydrocarbon group with 6 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or
O, S or NR³⁰¹;
L³⁰³ each independently denotes
a linear, branched or cyclic divalent to tetravalent aliphatic hydrocarbon group with 1 to 18 carbon atoms optionally substituted with fluorine, or
O or S;
Rf³⁰¹ each independently denotes
a moiety with 2 or more carbon atoms containing 3 or more fluorine atoms;
R³⁰¹ each independently denotes
an optionally substituted monocyclic, linked or condensed monovalent aromatic hydrocarbon group with 6 to 26 carbon atoms,
an optionally substituted monocyclic, linked or condensed monovalent heteroaromatic group with 3 to 26 carbon atoms,
an optionally substituted cyclic monovalent aliphatic hydrocarbon group with 3 to 18 carbon atoms, or
a fluoroalkyl structure with 1 or more carbon atoms containing 3 or more fluorine atoms;
X³⁰¹ denotes
an optionally substituted monocyclic, linked or condensed divalent to tetravalent aromatic hydrocarbon group with 3 to 26 carbon atoms,
an optionally substituted monocyclic, linked or condensed divalent to tetravalent heteroaromatic group with 3 to 26 carbon atoms,
an optionally substituted cyclic divalent to tetravalent aliphatic hydrocarbon group with 3 to 18 carbon atoms,
an optionally substituted cyclic divalent to tetravalent heteroaliphatic hydrocarbon group with 3 to 18 carbon atoms, or O;
gg each independently denotes an integer in the range of 0 to 6,
hh each independently denotes an integer in the range of 0 to 12,
ii each independently denotes an integer in the range of 1 to 6,
jj each independently denotes an integer in the range of 0 to 6,
kk each independently denotes an integer in the range of 1 to 6,
mm each independently denotes an integer in the range of 1 to 6, or
nn each independently denotes an integer in the range of 1 to 6,
in the formula (301), when L³⁰¹ is NR³⁰¹, and R³⁰¹ is an optionally substituted monocyclic, linked or condensed monovalent aromatic hydrocarbon group with 6 to 26 carbon atoms, L³⁰¹ is each independently substituted with a fluorine atom or a moiety with 1 or more carbon atoms containing 3 or more fluorine atoms,
when X³⁰¹ is a p-phenylene group and nn is 1, the p-phenylene group is substituted with a fluorine atom or a monovalent substituent with 1 or more carbon atoms optionally containing 3 or more fluorine atoms, and
X³⁰¹ does not have a cyano group, a linear vinylene group, a ketone group, a thioketone group or a selenium atom.

22. The heterocyclic compound according to claim 21, wherein Rf³⁰¹ is represented by the following formula (311): 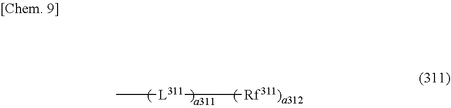
wherein
Rf³¹¹ each independently denotes
a linear, branched or cyclic aliphatic hydrocarbon group with 1 to 18 carbon atoms optionally substituted with a fluorine atom;
L³¹¹ each independently denotes
a linear, branched or cyclic divalent aliphatic hydrocarbon group with 1 to 18 carbon atoms optionally substituted with O, S or a fluorine atom, or
a vinylene group that may form a ring;
a311 each independently denotes an integer in the range of 0 to 4; and
a312 each independently denotes an integer in the range of 1 to 4.

23. A thin film for metal patterning comprising a material for metal patterning and capable of patterning a metal film or a metal multilayer film, wherein
the material for metal patterning contains a compound represented by the formula (A1) or (B1) according to claim 1, and
the metal film or the metal multilayer film contains at least one selected from the group consisting of ytterbium, magnesium, silver, aluminum and an alloy of magnesium and silver.

24. A thin film for metal patterning comprising the material for metal patterning according to claim 1 wherein a water contact angle is 90 degrees or more.

25. An organic electroluminescent element comprising a negative electrode, wherein
the negative electrode
contains at least one selected from the group consisting of ytterbium, magnesium, silver, aluminum and an alloy of magnesium and silver, and
is patterned with a material for metal patterning, and
the material for metal patterning contains a compound represented by the formula (A1) or (B1) according to claim 1.

26. A method for forming a metal pattern, comprising:
forming an organic material pattern containing the material for metal patterning according to claim 1; and
applying a metallic material to a region where the organic material pattern is formed and a region where the organic material pattern is not formed to form a metal pattern in the region where the organic material pattern is not formed.

27. An electronic device comprising the material for metal patterning according to claim 1.
